(19) United States
(12) Patent Application Publication
(10) Pub. No.: US 2026/0034123 A1
(43) Pub. Date: Feb. 5, 2026

(54) CDK2 INHIBITORS AND METHODS OF USING THE SAME

(71) Applicant: Cedilla Therapeutics, Inc., Foxboro, MA (US)

(72) Inventors: Louise Clare KIRMAN, Foxboro, MA (US); Carl Eric SCHWARTZ, Foxboro, MA (US); Thomas P. BLAISDELL, Foxboro, MA (US)

(21) Appl. No.: 18/995,332

(22) PCT Filed: Jul. 28, 2023
(86) PCT No.: PCT/US2023/071255

§ 371 Date: Jan. 16, 2025

Related U.S. Application Data
(60) Provisional application No. 63/393,074, filed on Jul. 28, 2022.
(60) Provisional application No. 63/503,855, filed on May 23, 2023.

(51) Int. Cl. A61K 31/4985; A61K 31/4155; A61K 31/427; A61K 31/428; A61K 31/4355; A61K 31/437; A61K 31/4439; A61K 31/497; C07D 487/10; C07D 519/00

(52) U.S. Cl. CPC A61K 31/4985; A61K 31/4155; A61K 31/427; A61K 31/428; A61K 31/4355; A61K 31/437; A61K 31/4439; A61K 31/497; C07D 487/10; C07D 519/00

(57) ABSTRACT

The present disclosure provides compounds, compositions thereof, and methods of using the same for the inhibition of CDK2, and the treatment of CDK2 related diseases and disorders.

### CROSS REFERENCE TO RELATED APPLICATIONS

This application claims the benefit of priority to U.S. Provisional Application No. 63/393,074, filed Jul. 28, 2022 and U.S. Provisional Application No. 63/503,855, filed May 23, 2023, the entire contents of each of which are herein incorporated by reference.

### FIELD

The present disclosure relates generally to Cyclin-dependent kinase 2 (CDK2) inhibiting chemical compounds and uses thereof in the inhibition of the activity of CDK2. The disclosure also provides pharmaceutically acceptable compositions comprising compounds disclosed herein and methods of using said compounds and compositions in the treatment of various disorders related to CDK2 activity.

### BACKGROUND

Cell cycle dysregulation, including uncontrolled cell growth, impaired cell differentiation and abnormal apoptosis have been shown to be caused by over activity of Cyclin-dependent kinases (CDKs). CDKs are important serine/threonine protein kinases that become active when combined with a specific cyclin partner. There are various subtypes of CDKs, each having a different role during the cell cycle, with varying levels of activity during each of the phases. CDK1, CDK2, CDK4 and CDK6 have been found to be specifically important subtypes, where overactivity of one or more of these subtypes may lead to dysregulation of the cell cycle and the development of a variety of cancers. The S phase of the cell cycle is responsible for DNA replication and is the phase where aberrant DNA replication may occur. The CDK2/cyclin E complex is required for the cell cycle transition from the G1 phase to the S phase, and the CDK2/cyclin A complex is required for the cell cycle transition from the S phase to the G2 phase. Therefore, selective inhibition of the CDK2/cyclin E and/or CDK2/cyclin A complexes can prevent aberrant DNA replication and can be used to treat certain cancers.

Accordingly, there is a need for the development of compounds capable of inhibiting the activity of CDK2/cyclin complexes, and pharmaceutical compositions thereof, for the prevention, and treatment of CDK2 related diseases or disorders.

### SUMMARY

The present disclosure is based at least in part on the identification of compounds that bind and inhibit Cyclin-dependent kinase 2 (CDK2) and/or CDK2/cyclin complexes and methods of using the same to treat diseases associated with CDK2 activity. Disclosed herein is a compound according to Formula I, Formula I′ or a pharmaceutically acceptable salt thereof:

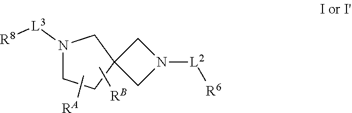

wherein each variable is as defined and described herein.

Compounds of the present disclosure, and pharmaceutically acceptable compositions thereof, are useful for treating a variety of diseases, disorders or conditions, associated with CDK2 activity. Such diseases, disorders, or conditions include those described herein.

### DETAILED DESCRIPTION OF CERTAIN EMBODIMENTS

### 1. General Description of Compounds of the Disclosure

The present disclosure provides compounds capable of inhibiting Cyclin-dependent kinase 2 (CDK2) and/or CDK2/cyclin complexes.

In certain embodiments, the present disclosure provides inhibitors of CDK2 activity. In some embodiments, the inhibitors of CDK2 include compounds of Formula I:

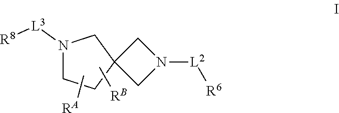

or a pharmaceutically acceptable salt thereof, wherein:
R^{A} is

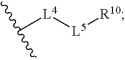

R^{B} is a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹; and provided that if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 3-7 membered saturated, partially unsaturated, or heteroaryl ring (having 0-3 heteroatoms independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In certain aspects, the present disclosure provides inhibitors of CDK2 activity. In some embodiments, the inhibitors of CDK2 include compounds of Formula I′:

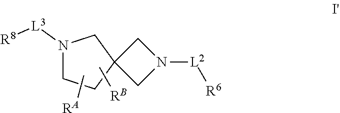

or a pharmaceutically acceptable salt thereof, wherein:
R^{A} is

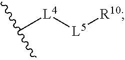

R^{B} is a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 5-12 membered saturated or partially unsaturated bicyclic carbocyclic ring that is optionally bridged, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with x instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with y instances of R⁹; and provided that if L⁵ is a saturated or unsaturated, straight or branched, optionally substituted bivalent C₂ hydrocarbon chain wherein said 1^{st} methylene unit of L⁵ is replaced with said bivalent cyclic group, a 2ⁿd methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —C(O)N(R)S(O)₂R, —C(O)N(R)S(O)₂NR₂, an optionally substituted C₁₋₆ aliphatic group, an optionally substituted C₁₋₆ aliphatic-Cy group, or Cy;
R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with z instances of R⁹;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 3-7 membered saturated, partially unsaturated, or heteroaryl ring (having 0-3 heteroatoms, in addition to the nitrogen, independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
x is 0, 1, 2, 3, 4, 5, 6, 7, or 8;
y is 0, 1, 2, 3, 4, 5, 6, 7, or 8; and
z is 0, 1, 2, 3, 4, 5, 6, 7, or 8.

Overexpression of CDK2 is associated with abnormal regulation of the cell-cycle. The cyclin E/CDK2 complex plays an important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells, and CDK2 inhibition has been reported to restore sensitivity to tamoxifen or CDK4 inhibitors in tamoxifen-resistant and CCNE2 overexpressing cells. (Caldon et al., Mol. Cancer Ther. (2012) 11:1488-99; Herrera-Abreu et al., Cancer Res. (2016) 76: 2301-2313). Cyclin E amplification also reportedly contributes to trastuzumab resistance in H-ER2+ breast cancer. (Scaltriti et al., Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Breast Care (2011) 6:273-278; Alexander et al., Oncotarget (2017) 8: 14897-14911.)

Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116: 2621-34; Etemadmoghadam et al., Clin Cancer Res (2013) 19: 5960-71; Au-Yeung et al., Clin. Cancer Res. (2017) 23:1862-1874; Ayhan et al., Modern Pathology (2017) 30: 297-303; Ooi et al., Hum Pathol. (2017) 61: 58-67; Noske et al., Oncotarget (2017) 8: 14794-14805).

There remains a need in the art for CDK inhibitors, especially selective CDK2 inhibitors, which may be useful for the treatment of cancer or other proliferative diseases or conditions. In particular, CDK2 inhibitors may be useful in treating CCNE1 or CCNE2 amplified tumors.

### 2. Compounds and Definitions

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry,” Thomas Sorrell, University Science Books, Sausalito: 2005, and “March's Advanced Organic Chemistry: Reactions Mechanisms and Structure,” 8^{th} Ed.: Smith, M. B., John Wiley & Sons, New York: 2019, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused, bridged, or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

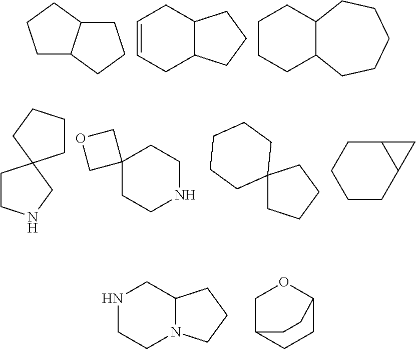

Exemplary bridged bicyclics, contemplated as falling under the scope of a “bicycle” or “bicyclic ring” include:

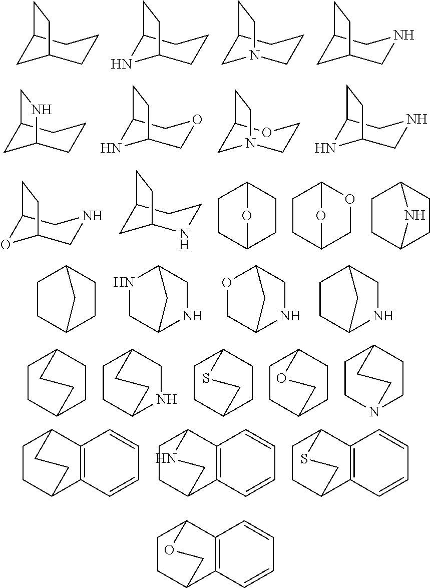

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or 1.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Covalent substituents on a substitutable carbon atom of an “optionally substituted” group are independently halogen; —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)O—CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂, —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆N(H)OR^{∘}; —(CH₂)₀₋₆C(O)R^{∘}; —C(S)R^{∘}; —(CH₂)₀₋₆C(O)OR^{∘}; —(CH₂)₀₋₆—C(O)SR^{∘}; —(CH₂)₀₋₆C(O)OSiR^{∘}₃; —(CH₂)₀₋₆OC(O)R^{∘}; —OC(O)(CH₂)₀₋₆SR^{∘}, —(CH₂)₀₋₆SC(O)R^{∘}; —(CH₂)₀₋₆C(O)NR^{∘}₂, —C(S)NR^{∘}₂; —C(S)SR^{∘}; —SC(S)SR^{∘}, —(CH₂)₀₋₆OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆SR^{∘}; —(CH₂)₀₋₆S(O)₂R^{∘}; —(CH₂)₀₋₆S(O)₂OR^{∘}; —(CH₂)₀₋₆OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted, substituted with one or more of independently selected methyl or —CO₂H, or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{∘} include ═0 and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†)S(O)}₂R^{†}; wherein each Rt is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of Rt are independently halogen, —R^{●}, -(haloR^{●}), —OH, —OR^{●}, —O(haloR^{●}), —CN, —C(O)OH, —C(O)O^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

“One or more instances” or “one or more” as referencing substitutions, as used herein, refers to, for example, 1, 2, 3, 4, 5, 6, 7, etc. instances of substitution of functional groups, which may each be independently selected, on a chemical moiety to which “one or more” instances of substitution refers. It is to be understood that any “optionally substituted” moiety, may be substituted with “one or more” optional substituents each independently selected from those optional substituents as described herein.

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “inhibitor” is defined as a compound that binds to and/or inhibits CDK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM, when measured in an appropriate assay.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

As used herein, the term “inhibitorily active metabolite or residue thereof” means that a metabolite or residue thereof is also an inhibitor of a CDK2 protein, or a mutant thereof.

### 3. Description of Exemplary Embodiments

In certain embodiments, the present disclosure provides inhibitors of CDK2 activity. In some embodiments, the inhibitors of CDK2 include compounds of Formula I:

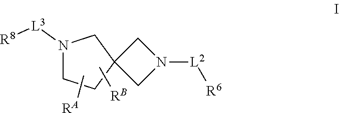

or a pharmaceutically acceptable salt thereof, wherein:
R^{A} is

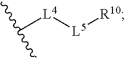

R^{B} is a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
L⁵ is a covalent bond or a saturated or unsaturated, straight or branched, optionally substituted bivalent C₁₋₂ hydrocarbon chain, wherein a j^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹; and wherein if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 3-7 membered saturated, partially unsaturated, or heteroaryl ring (having 0-3 heteroatoms, in addition to the nitrogen, independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In certain embodiments, the present disclosure provides inhibitors of CDK2 activity. In some embodiments, the inhibitors of CDK2 include compounds of Formula I′:

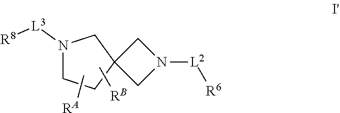

or a pharmaceutically acceptable salt thereof, wherein:
R^{A} is

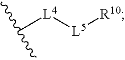

R^{B} is a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 5-12 membered saturated or partially unsaturated bicyclic carbocyclic ring that is optionally bridged, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with x instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with y instances of R⁹; and provided that if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —C(O)N(R)S(O)₂R, —C(O)N(R)S(O)₂NR₂, an optionally substituted C₁₋₆ aliphatic group, an optionally substituted C₁₋₆ aliphatic-Cy group, or Cy;
R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with z instances of R⁹;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 3-7 membered saturated, partially unsaturated, or heteroaryl ring (having 0-3 heteroatoms, in addition to the nitrogen, independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
x is 0, 1, 2, 3, 4, 5, 6, 7, or 8;
y is 0, 1, 2, 3, 4, 5, 6, 7, or 8; and
z is 0, 1, 2, 3, 4, 5, 6, 7, or 8.

As defined generally above, R^{A} is

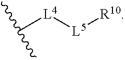

In some embodiments, R^{A} is

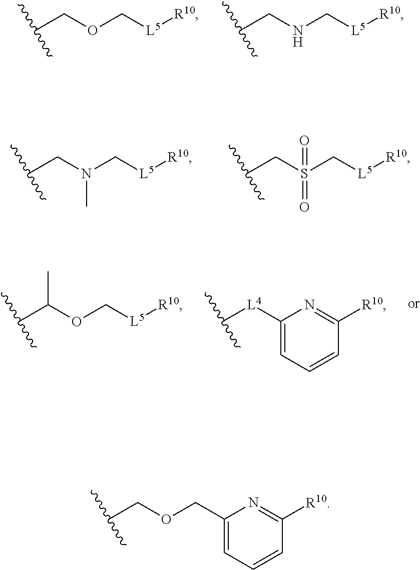

In some embodiments, R^{A} is selected from one of the substituents of Table 1. In some embodiments, R^{A} is selected from those depicted in the compounds of Table 8A, below. In some embodiments, R^{A} is selected from those depicted in the compounds of Table 8B, below.

TABLE 1
R^{A} substituents

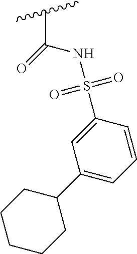 [SMILES: *C(=O)NS(=O)(=O)c1cccc(C2CCCCC2)c1]

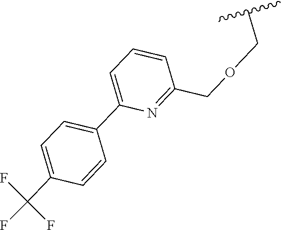 [SMILES: *COCc1cccc(-c2ccc(C(F)(F)F)cc2)n1]

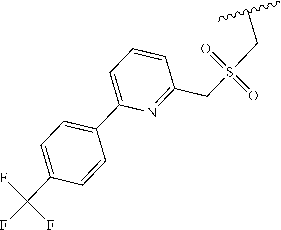 [SMILES: *CS(=O)(=O)Cc1cccc(-c2ccc(C(F)(F)F)cc2)n1]

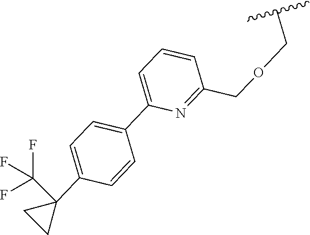 [SMILES: *COCc1cccc(-c2ccc(C3(C(F)(F)F)CC3)cc2)n1]

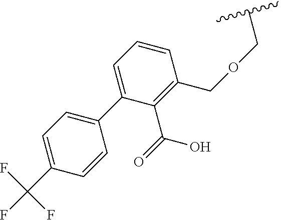 [SMILES: *COCc1cccc(-c2ccc(C(F)(F)F)cc2)c1C(=O)O]

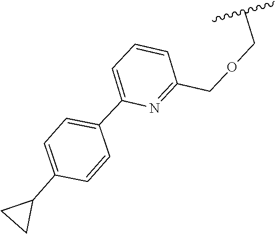 [SMILES: *COCc1cccc(-c2ccc(C3CC3)cc2)n1]

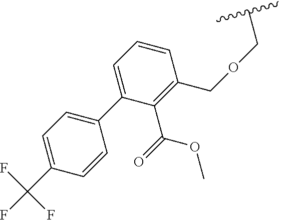 [SMILES: *COCc1cccc(-c2ccc(C(F)(F)F)cc2)c1C(=O)OC]

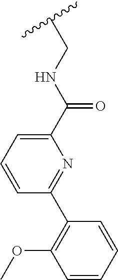 [SMILES: *CNC(=O)c1cccc(-c2ccccc2OC)n1]

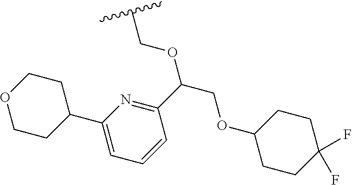 [SMILES: *COC(COC1CCC(F)(F)CC1)c1cccc(C2CCOCC2)n1]

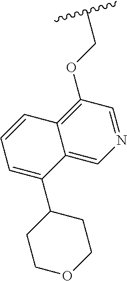 [SMILES: *COc1cncc2c(C3CCOCC3)cccc12]

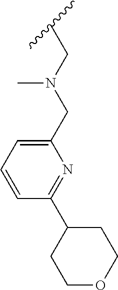 [SMILES: *CN(C)Cc1cccc(C2CCOCC2)n1]

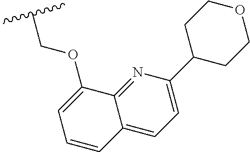 [SMILES: *COc1cccc2ccc(C3CCOCC3)nc12]

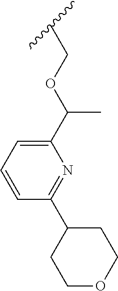 [SMILES: *COC(C)c1cccc(C2CCOCC2)n1]

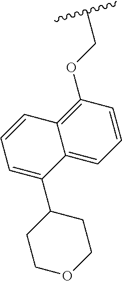 [SMILES: *COc1cccc2c(C3CCOCC3)cccc12]

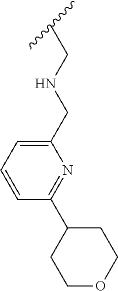 [SMILES: *CNCc1cccc(C2CCOCC2)n1]

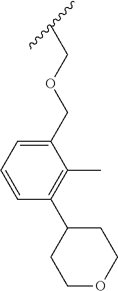 [SMILES: *COCc1cccc(C2CCOCC2)c1C]

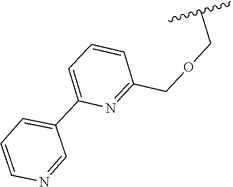 [SMILES: *COCc1cccc(-c2cccnc2)n1]

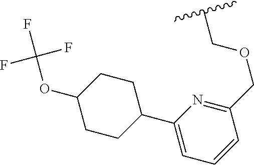 [SMILES: *COCc1cccc(C2CCC(OC(F)(F)F)CC2)n1]

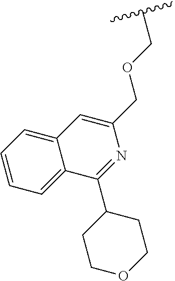 [SMILES: *COCc1cc2ccccc2c(C2CCOCC2)n1]

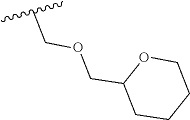 [SMILES: *COCC1CCCCO1]

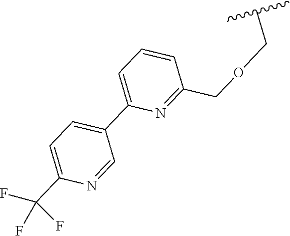 [SMILES: *COCc1cccc(-c2ccc(C(F)(F)F)nc2)n1]

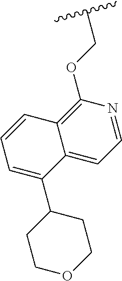 [SMILES: *COc1nccc2c(C3CCOCC3)cccc12]

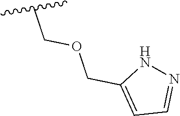 [SMILES: *COCc1ccn[nH]1]

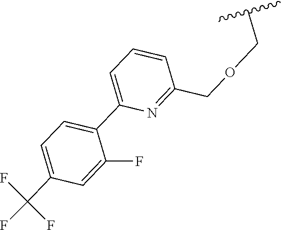 [SMILES: *COCc1cccc(-c2ccc(C(F)(F)F)cc2F)n1]

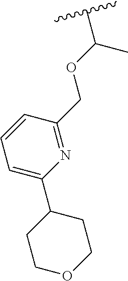 [SMILES: *C(C)OCc1cccc(C2CCOCC2)n1]

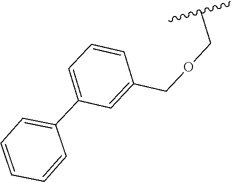 [SMILES: *COCc1cccc(-c2ccccc2)c1]

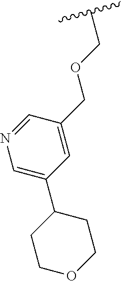 [SMILES: *COCc1cncc(C2CCOCC2)c1]

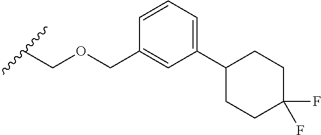 [SMILES: *COCc1cccc(C2CCC(F)(F)CC2)c1]

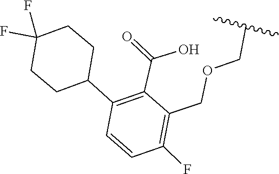 [SMILES: *COCc1c(F)ccc(C2CCC(F)(F)CC2)c1C(=O)O]

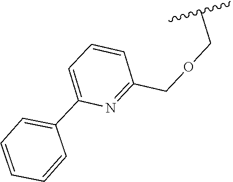 [SMILES: *COCc1cccc(-c2ccccc2)n1]

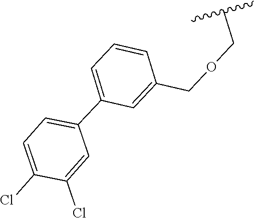 [SMILES: *COCc1cccc(-c2ccc(Cl)c(Cl)c2)c1]

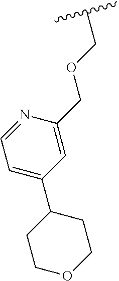 [SMILES: *COCc1cc(C2CCOCC2)ccn1]

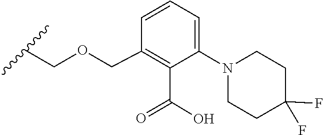 [SMILES: *COCc1cccc(N2CCC(F)(F)CC2)c1C(=O)O]

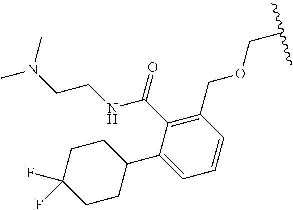 [SMILES: *COCc1cccc(C2CCC(F)(F)CC2)c1C(=O)NCCN(C)C]

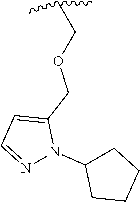 [SMILES: *COCc1ccnn1C1CCCC1]

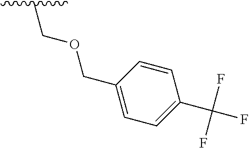 [SMILES: *COCc1ccc(C(F)(F)F)cc1]

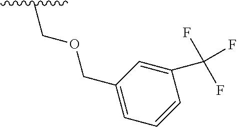 [SMILES: *COCc1cccc(C(F)(F)F)c1]

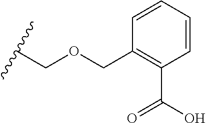 [SMILES: *COCc1ccccc1C(=O)O]

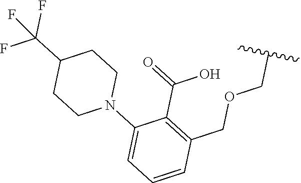 [SMILES: *COCc1cccc(N2CCC(C(F)(F)F)CC2)c1C(=O)O]

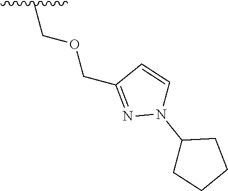 [SMILES: *COCc1ccn(C2CCCC2)n1]

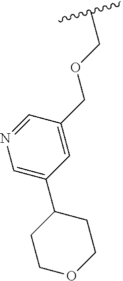 [SMILES: *COCc1cncc(C2CCOCC2)c1]

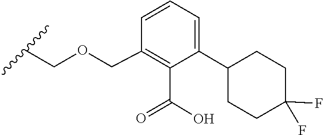 [SMILES: *COCc1cccc(C2CCC(F)(F)CC2)c1C(=O)O]

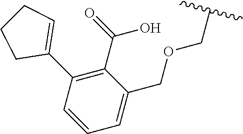 [SMILES: *COCc1cccc(C2=CCCC2)c1C(=O)O]

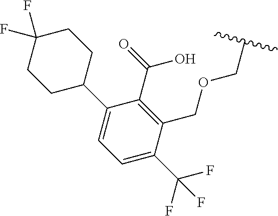 [SMILES: *COCc1c(C(F)(F)F)ccc(C2CCC(F)(F)CC2)c1C(=O)O]

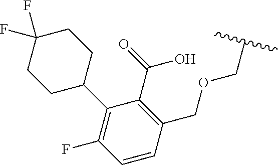 [SMILES: *COCc1ccc(F)c(C2CCC(F)(F)CC2)c1C(=O)O]

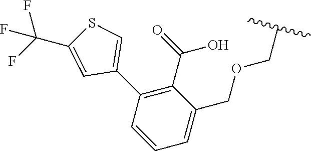 [SMILES: *COCc1cccc(-c2csc(C(F)(F)F)c2)c1C(=O)O]

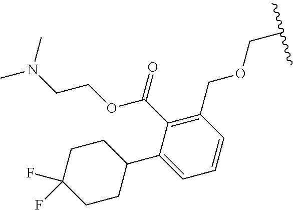 [SMILES: *COCc1cccc(C2CCC(F)(F)CC2)c1C(=O)OCCN(C)C]

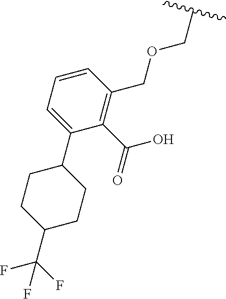 [SMILES: *COCc1cccc(C2CCC(C(F)(F)F)CC2)c1C(=O)O]

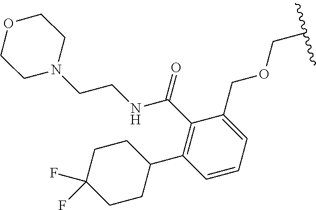 [SMILES: *COCc1cccc(C2CCC(F)(F)CC2)c1C(=O)NCCN1CCOCC1]

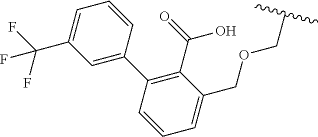 [SMILES: *COCc1cccc(-c2cccc(C(F)(F)F)c2)c1C(=O)O]

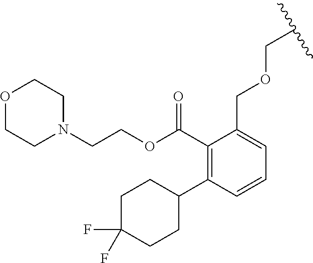 [SMILES: *COCc1cccc(C2CCC(F)(F)CC2)c1C(=O)OCCN1CCOCC1]

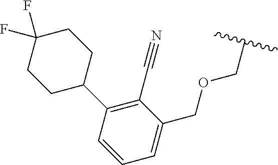 [SMILES: *COCc1cccc(C2CCC(F)(F)CC2)c1C#N]

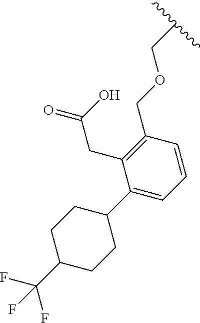 [SMILES: *COCc1cccc(C2CCC(C(F)(F)F)CC2)c1CC(=O)O]

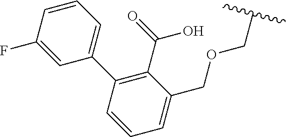 [SMILES: *COCc1cccc(-c2cccc(F)c2)c1C(=O)O]

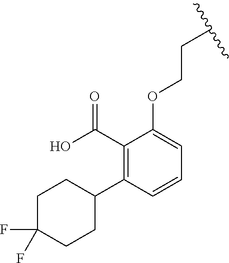 [SMILES: *CCOc1cccc(C2CCC(F)(F)CC2)c1C(=O)O]

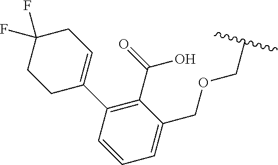 [SMILES: *COCc1cccc(C2=CCC(F)(F)CC2)c1C(=O)O]

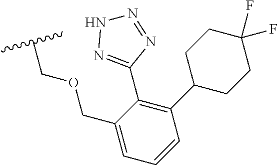 [SMILES: *COCc1cccc(C2CCC(F)(F)CC2)c1-c1nn[nH]n1]

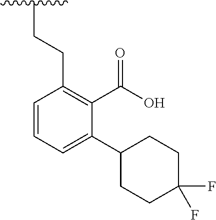 [SMILES: *CCc1cccc(C2CCC(F)(F)CC2)c1C(=O)O]

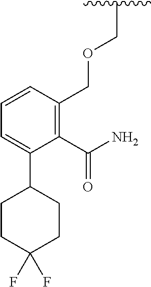 [SMILES: *COCc1cccc(C2CCC(F)(F)CC2)c1C(N)=O]

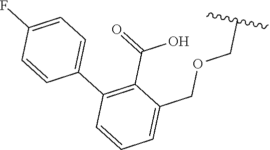 [SMILES: *COCc1cccc(-c2ccc(F)cc2)c1C(=O)O]

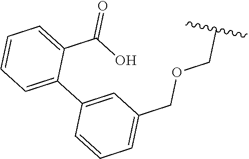 [SMILES: *COCc1cccc(-c2ccccc2C(=O)O)c1]

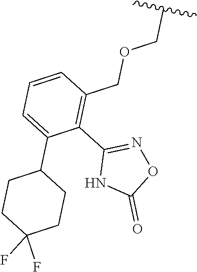 [SMILES: *COCc1cccc(C2CCC(F)(F)CC2)c1-c1noc(=O)[nH]1]

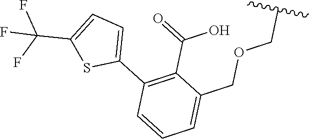 [SMILES: *COCc1cccc(-c2ccc(C(F)(F)F)s2)c1C(=O)O]

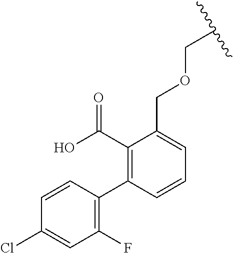 [SMILES: *COCc1cccc(-c2ccc(Cl)cc2F)c1C(=O)O]

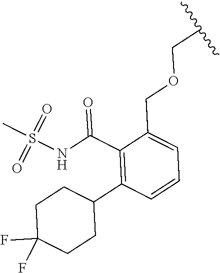 [SMILES: *COCc1cccc(C2CCC(F)(F)CC2)c1C(=O)NS(C)(=O)=O]

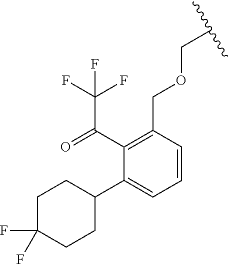 [SMILES: *COCc1cccc(C2CCC(F)(F)CC2)c1C(=O)C(F)(F)F]

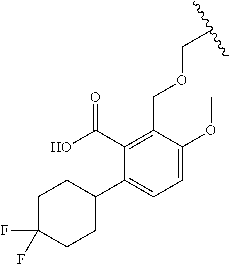 [SMILES: *COCc1c(OC)ccc(C2CCC(F)(F)CC2)c1C(=O)O]

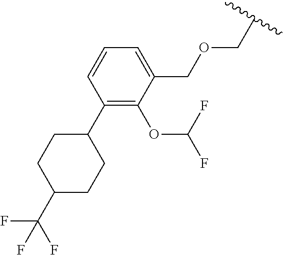 [SMILES: *COCc1cccc(C2CCC(C(F)(F)F)CC2)c1OC(F)F]

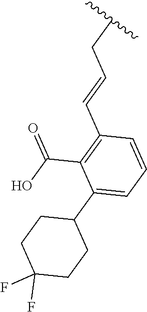 [SMILES: *C/C=C/c1cccc(C2CCC(F)(F)CC2)c1C(=O)O]

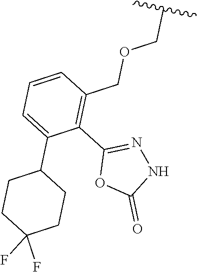 [SMILES: *COCc1cccc(C2CCC(F)(F)CC2)c1-c1n[nH]c(=O)o1]

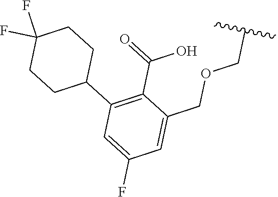 [SMILES: *COCc1cc(F)cc(C2CCC(F)(F)CC2)c1C(=O)O]

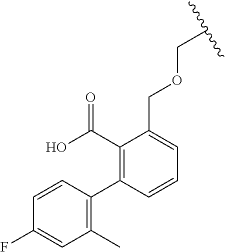 [SMILES: *COCc1cccc(-c2ccc(F)cc2C)c1C(=O)O]

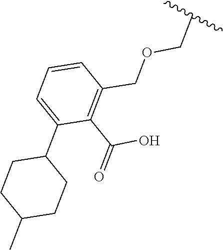 [SMILES: *COCc1cccc(C2CCC(C)CC2)c1C(=O)O]

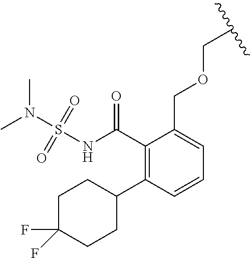 [SMILES: *COCc1cccc(C2CCC(F)(F)CC2)c1C(=O)NS(=O)(=O)N(C)C]

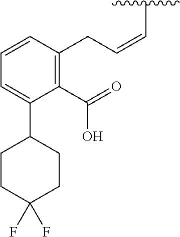 [SMILES: */C=C\Cc1cccc(C2CCC(F)(F)CC2)c1C(=O)O]

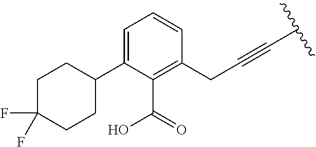 [SMILES: *C#CCc1cccc(C2CCC(F)(F)CC2)c1C(=O)O]

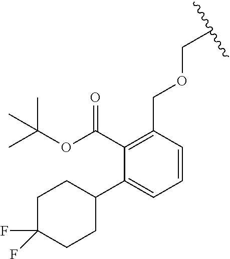 [SMILES: *COCc1cccc(C2CCC(F)(F)CC2)c1C(=O)OC(C)(C)C]

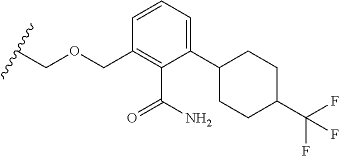 [SMILES: *COCc1cccc(C2CCC(C(F)(F)F)CC2)c1C(N)=O]

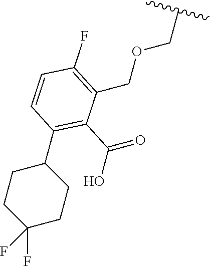 [SMILES: *COCc1c(F)ccc(C2CCC(F)(F)CC2)c1C(=O)O]

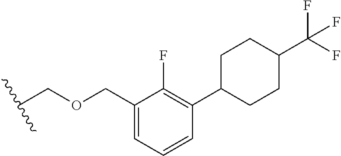 [SMILES: *COCc1cccc(C2CCC(C(F)(F)F)CC2)c1F]

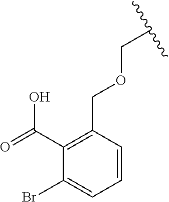 [SMILES: *COCc1cccc(Br)c1C(=O)O]

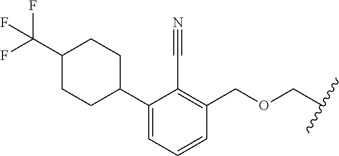 [SMILES: *COCc1cccc(C2CCC(C(F)(F)F)CC2)c1C#N]

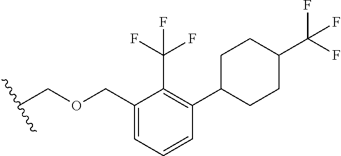 [SMILES: *COCc1cccc(C2CCC(C(F)(F)F)CC2)c1C(F)(F)F]

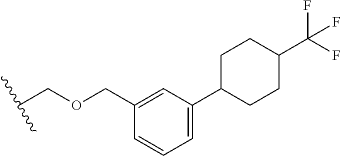 [SMILES: *COCc1cccc(C2CCC(C(F)(F)F)CC2)c1]

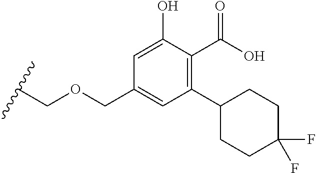 [SMILES: *COCc1cc(O)c(C(=O)O)c(C2CCC(F)(F)CC2)c1]

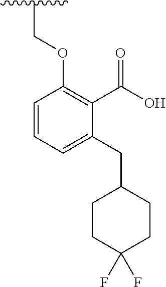 [SMILES: *COc1cccc(CC2CCC(F)(F)CC2)c1C(=O)O]

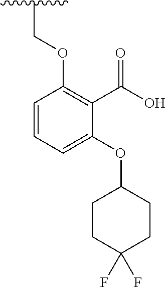 [SMILES: *COc1cccc(OC2CCC(F)(F)CC2)c1C(=O)O]

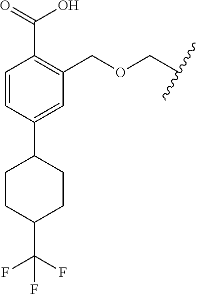 [SMILES: *COCc1cc(C2CCC(C(F)(F)F)CC2)ccc1C(=O)O]

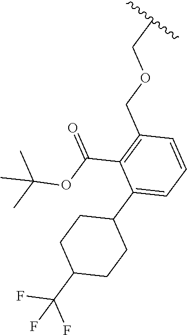 [SMILES: *COCc1cccc(C2CCC(C(F)(F)F)CC2)c1C(=O)OC(C)(C)C]

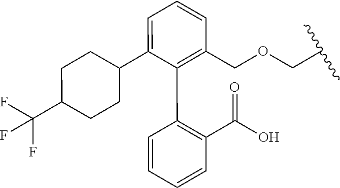 [SMILES: *COCc1cccc(C2CCC(C(F)(F)F)CC2)c1-c1ccccc1C(=O)O]

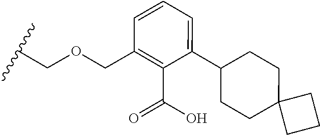 [SMILES: *COCc1cccc(C2CCC3(CCC3)CC2)c1C(=O)O]

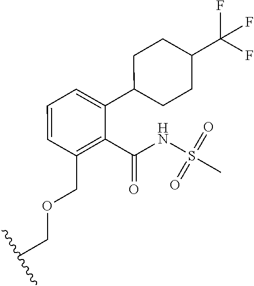 [SMILES: *COCc1cccc(C2CCC(C(F)(F)F)CC2)c1C(=O)NS(C)(=O)=O]

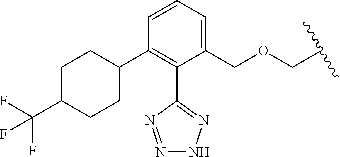 [SMILES: *COCc1cccc(C2CCC(C(F)(F)F)CC2)c1-c1nn[nH]n1]

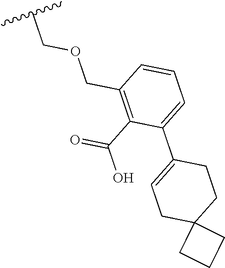 [SMILES: *COCc1cccc(C2=CCC3(CCC3)CC2)c1C(=O)O]

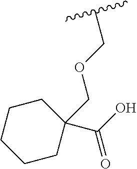 [SMILES: *COCC1(C(=O)O)CCCCC1]

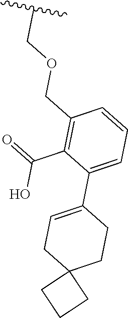 [SMILES: *COCc1cccc(C2=CCC3(CCC3)CC2)c1C(=O)O]

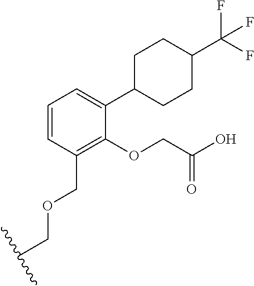 [SMILES: *COCc1cccc(C2CCC(C(F)(F)F)CC2)c1OCC(=O)O]

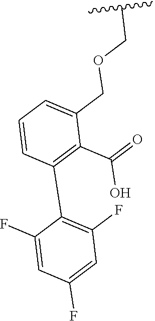 [SMILES: *COCc1cccc(-c2c(F)cc(F)cc2F)c1C(=O)O]

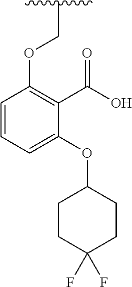 [SMILES: *COc1cccc(OC2CCC(F)(F)CC2)c1C(=O)O]

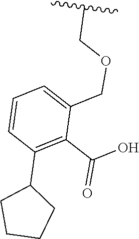 [SMILES: *COCc1cccc(C2CCCC2)c1C(=O)O]

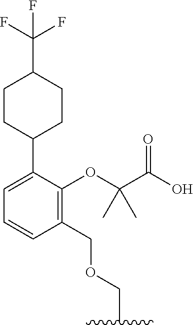 [SMILES: *COCc1cccc(C2CCC(C(F)(F)F)CC2)c1OC(C)(C)C(=O)O]

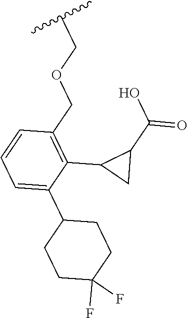 [SMILES: *COCc1cccc(C2CCC(F)(F)CC2)c1C1CC1C(=O)O]

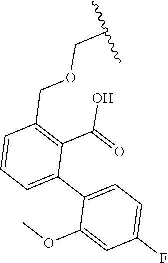 [SMILES: *COCc1cccc(-c2ccc(F)cc2OC)c1C(=O)O]

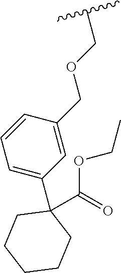 [SMILES: *COCc1cccc(C2(C(=O)OCC)CCCCC2)c1]

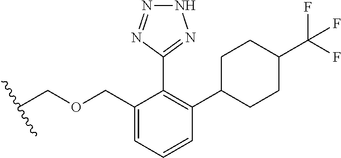 [SMILES: *COCc1cccc(C2CCC(C(F)(F)F)CC2)c1-c1nn[nH]n1]

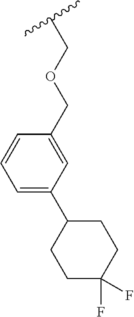 [SMILES: *COCc1cccc(C2CCC(F)(F)CC2)c1]

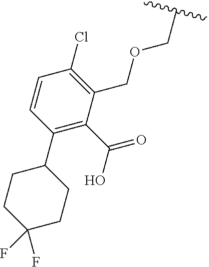 [SMILES: *COCc1c(Cl)ccc(C2CCC(F)(F)CC2)c1C(=O)O]

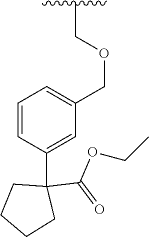 [SMILES: *COCc1cccc(C2(C(=O)OCC)CCCC2)c1]

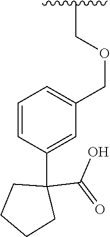 [SMILES: *COCc1cccc(C2(C(=O)O)CCCC2)c1]

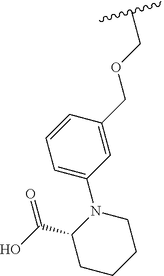 [SMILES: *COCc1cccc(N2CCCC[C@@H]2C(=O)O)c1]

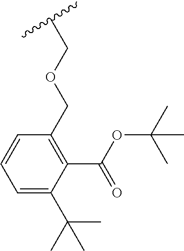 [SMILES: *COCc1cccc(C(C)(C)C)c1C(=O)OC(C)(C)C]

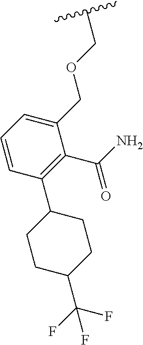 [SMILES: *COCc1cccc(C2CCC(C(F)(F)F)CC2)c1C(N)=O]

As defined generally above, R^{B} is a hydrogen, an optionally substituted C₁₋₆ aliphatic group, or a halogen. In some embodiments, R^{B} is a hydrogen. In some embodiments, R^{B} is an optionally substituted C₁₋₆ aliphatic group or a halogen. In some embodiments, R^{B} is an optionally substituted C₁₋₆ aliphatic group. In some embodiments, R^{B} is an optionally substituted methyl group. In some embodiments, R^{B} is a methyl group. In some embodiments, R^{B} is a halogen. In some embodiments, R^{B} is a F. In some embodiments, R^{B} is selected from those depicted in the compounds of Table 8A, below. In some embodiments, R^{B} is selected from those depicted in the compounds of Table 8B, below.

In some embodiments, R^{A} and R^{B} are geminally attached to the same carbon.

As defined generally above, L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—

In some embodiments, L⁴ is a saturated or unsaturated, straight or branched, optionally substituted bivalent C₁₋₃ hydrocarbon chain, wherein 0-3 methylene units of L⁴ are independently replaced by —O—, —NR—, —S(O)₂—, or —C(O)—. In some embodiments, L⁴ is a saturated or unsaturated, straight or branched, optionally substituted bivalent C₁₋₃ hydrocarbon chain, wherein 1 methylene units of L⁴ is independently replaced by —O—. In some embodiments, L⁴ is a saturated or unsaturated, straight or branched, optionally substituted bivalent C₁₋₃ hydrocarbon chain, wherein 1 methylene units of L⁴ is independently replaced by —N—. In some embodiments, L⁴ is a saturated or unsaturated, straight or branched, optionally substituted bivalent C₁₋₃ hydrocarbon chain, wherein 1 methylene units of L⁴ is independently replaced by —N(CH₃)—. In some embodiments, L⁴ is a saturated or unsaturated, straight or branched, optionally substituted bivalent C₁₋₃ hydrocarbon chain, wherein 1 methylene units of L⁴ is independently replaced by —S(O)₂—.

In some embodiments, L⁴ (as read from left to right

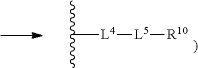

is —C(CH₃)H—O—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, —OCH₂—, or

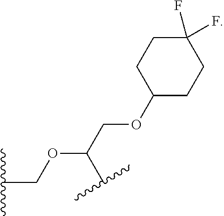 [SMILES: *COC(*)COC1CCC(F)(F)CC1]

As defined generally above, L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹; and wherein if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—

Also as defined generally above, L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with y instances of R⁹; and provided that if L⁵ is a saturated or unsaturated, straight or branched, optionally substituted bivalent C₂ hydrocarbon chain wherein said 1^{st} methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L⁵ is a covalent bond. In some embodiments, L⁵ is an optionally substituted bivalent C₁₋₂ hydrocarbon chain. In some embodiments, L⁵ is a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic heterocyclene ring (having 1-2 heteroatoms independently selected from nitrogen, oxygen, and sulfur), wherein the bivalent cyclic group is optionally substituted with one or more instances of R⁹.

In some embodiments, the L⁵ is a bivalent cyclic group substituted with 1 instance of R⁹. In some embodiments, L⁵ is a bivalent cyclic group substituted with 2 instances of R⁹. In some embodiments, L⁵ is a bivalent cyclic group substituted with 3 instances of R⁹. In some embodiments, L⁵ is a bivalent cyclic group substituted with 4 instances of R⁹. In some embodiments, L⁵ is a bivalent cyclic group substituted with 5 instances of R⁹.

In some embodiments L⁵ is selected from Table 2. In some embodiments the [symbol] on the left of the moiety in Table 2 connects to L⁴ and the [symbol] on the right of the moiety connects to R¹⁰. In some embodiments the on the [symbol] on the right of the moiety in Table 2 connects to L⁴ and the [symbol] on the left of the moiety connects to R¹⁰.

TABLE 2
Exemplary L⁵ Linkers

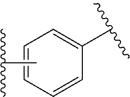

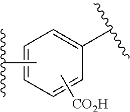

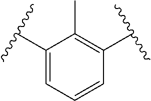 [SMILES: *c1cccc(*)c1C]

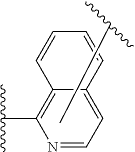

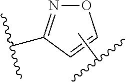

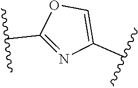 [SMILES: *c1coc(*)n1]

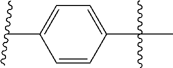 [SMILES: *c1ccc(*)cc1]

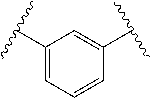 [SMILES: *c1cccc(*)c1]

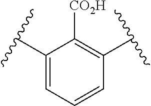 [SMILES: *c1cccc(*)c1C(=O)O]

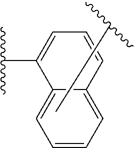

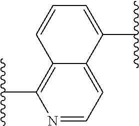 [SMILES: *c1cccc2c(*)nccc12]

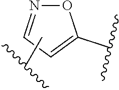

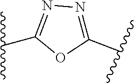 [SMILES: *c1nnc(*)o1]

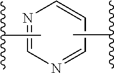

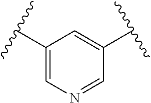 [SMILES: *c1cncc(*)c1]

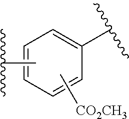

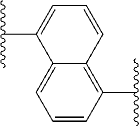 [SMILES: *c1cccc2c(*)cccc12]

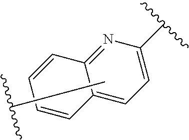

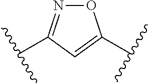 [SMILES: *c1cc(*)on1]

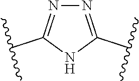 [SMILES: *c1nnc(*)[nH]1]

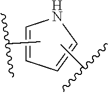

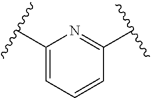 [SMILES: *c1cccc(*)n1]

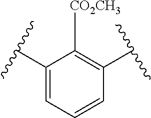 [SMILES: *c1cccc(*)c1C(=O)OC]

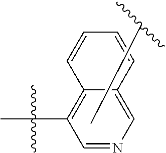

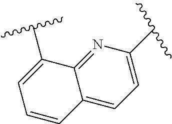 [SMILES: *c1ccc2cccc(*)c2n1]

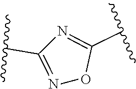 [SMILES: *c1noc(*)n1]

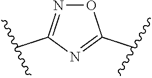 [SMILES: *c1noc(*)n1]

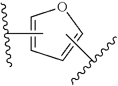

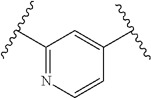 [SMILES: *c1ccnc(*)c1]

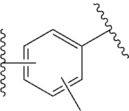

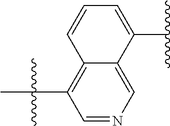 [SMILES: *c1cncc2c(*)cccc12]

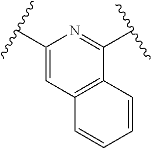 [SMILES: *c1cc2ccccc2c(*)n1]

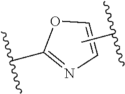

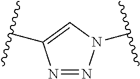 [SMILES: *c1cn(*)nn1]

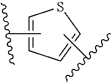

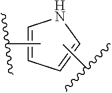

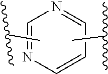

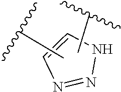

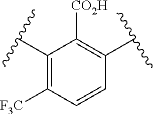 [SMILES: *c1ccc(C(F)(F)F)c(*)c1C(=O)O]

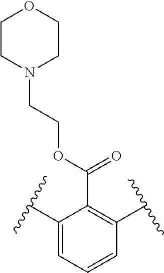 [SMILES: *c1cccc(*)c1C(=O)OCCN1CCOCC1]

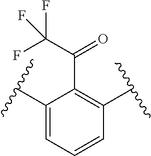 [SMILES: *c1cccc(*)c1C(=O)C(F)(F)F]

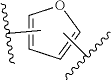

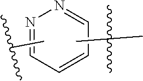

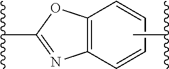

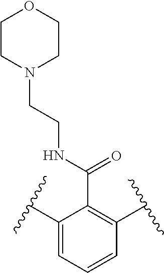 [SMILES: *c1cccc(*)c1C(=O)NCCN1CCOCC1]

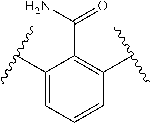 [SMILES: *c1cccc(*)c1C(N)=O]

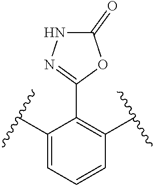 [SMILES: *c1cccc(*)c1-c1n[nH]c(=O)o1]

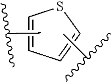

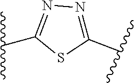 [SMILES: *c1nnc(*)s1]

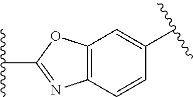 [SMILES: *c1ccc2nc(*)oc2c1]

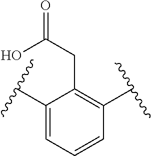 [SMILES: *c1cccc(*)c1CC(=O)O]

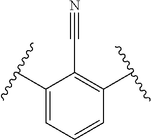 [SMILES: *c1cccc(*)c1C#N]

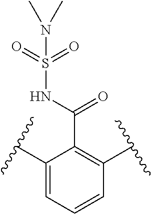 [SMILES: *c1cccc(*)c1C(=O)NS(=O)(=O)N(C)C]

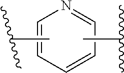

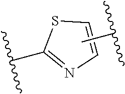

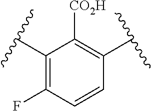 [SMILES: *c1ccc(F)c(*)c1C(=O)O]

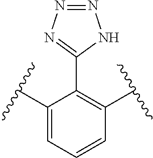 [SMILES: *c1cccc(*)c1-c1nnn[nH]1]

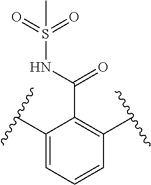 [SMILES: *c1cccc(*)c1C(=O)NS(C)(=O)=O]

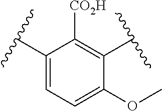 [SMILES: *c1ccc(OC)c(*)c1C(=O)O]

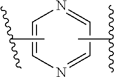

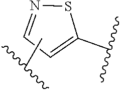

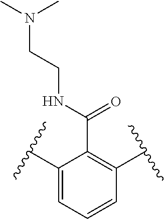 [SMILES: *c1cccc(*)c1C(=O)NCCN(C)C]

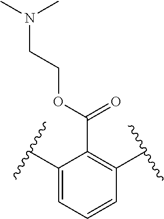 [SMILES: *c1cccc(*)c1C(=O)OCCN(C)C]

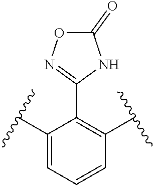 [SMILES: *c1cccc(*)c1-c1noc(=O)[nH]1]

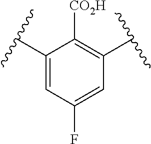 [SMILES: *c1cc(F)cc(*)c1C(=O)O]

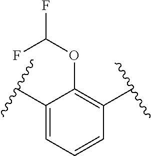 [SMILES: *c1cccc(*)c1OC(F)F]

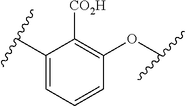 [SMILES: *Oc1cccc(*)c1C(=O)O]

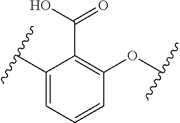 [SMILES: *Oc1cccc(*)c1C(=O)O]

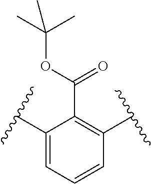 [SMILES: *c1cccc(*)c1C(=O)OC(C)(C)C]

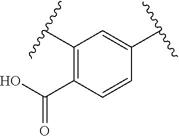 [SMILES: *c1ccc(C(=O)O)c(*)c1]

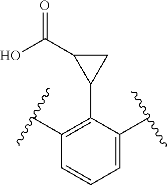 [SMILES: *c1cccc(*)c1C1CC1C(=O)O]

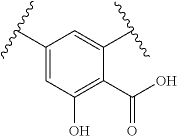 [SMILES: *c1cc(*)c(C(=O)O)c(O)c1]

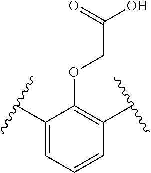 [SMILES: *c1cccc(*)c1OCC(=O)O]

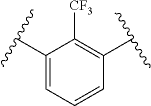 [SMILES: *c1cccc(*)c1C(F)(F)F]

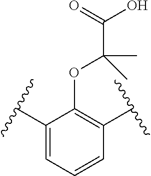 [SMILES: *c1cccc(*)c1OC(C)(C)C(=O)O]

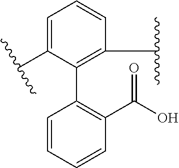 [SMILES: *c1cccc(*)c1-c1ccccc1C(=O)O]

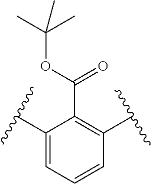 [SMILES: *c1cccc(*)c1C(=O)OC(C)(C)C]

As defined generally above, R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

Also as defined generally above, R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with z instances of R⁹.

In some embodiments, R¹⁰ is hydrogen. In some embodiments, R¹⁰ is halogen. In some embodiments, R¹⁰ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R¹⁰ is a cyclic group substituted with 1 instance of R⁹. In some embodiments, R¹⁰ is a cyclic group substituted with 2 instances of R⁹. In some embodiments, R¹⁰ is a cyclic group substituted with 3 instances of R⁹. In some embodiments, R¹⁰ is a cyclic group substituted with 4 instances of R⁹. In some embodiments, R¹⁰ is a cyclic group substituted with 5 instances of R⁹.

In some embodiments, R¹⁰ a cyclic group selected from cyclohexyl, phenyl, tetrahydropyranyl, pyridinyl, pyrazolyl, imidazolyl, oxazolyl, isoxazolyl, thiophenyl, pyrrolyl, and furanyl wherein the cyclic group is optionally substituted with one or more instances of R⁹. In some embodiments, R¹⁰ is selected from the groups of Table 3.

TABLE 3
Exemplary R¹⁰ groups

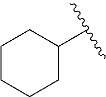 [SMILES: *C1CCCCC1]

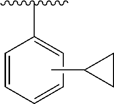

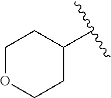 [SMILES: *C1CCOCC1]

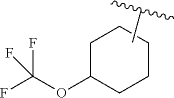

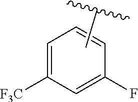

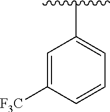 [SMILES: *c1cccc(C(F)(F)F)c1]

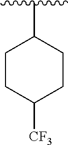 [SMILES: *C1CCC(C(F)(F)F)CC1]

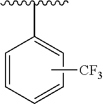

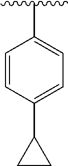 [SMILES: *c1ccc(C2CC2)cc1]

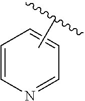

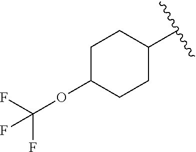 [SMILES: *C1CCC(OC(F)(F)F)CC1]

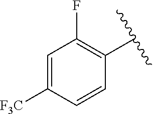 [SMILES: *c1ccc(C(F)(F)F)cc1F]

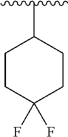 [SMILES: *C1CCC(F)(F)CC1]

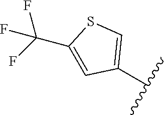 [SMILES: *c1csc(C(F)(F)F)c1]

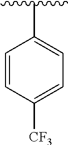 [SMILES: *c1ccc(C(F)(F)F)cc1]

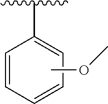

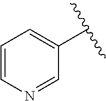 [SMILES: *c1cccnc1]

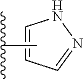

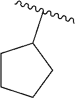 [SMILES: *C1CCCC1]

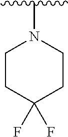 [SMILES: *N1CCC(F)(F)CC1]

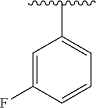 [SMILES: *c1cccc(F)c1]

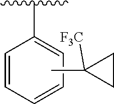

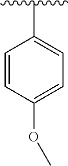 [SMILES: *c1ccc(OC)cc1]

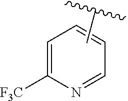

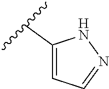 [SMILES: *c1ccn[nH]1]

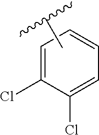

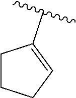 [SMILES: *C1=CCCC1]

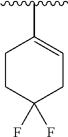 [SMILES: *C1=CCC(F)(F)CC1]

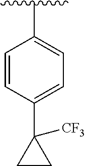 [SMILES: *c1ccc(C2(C(F)(F)F)CC2)cc1]

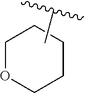

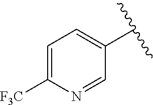 [SMILES: *c1ccc(C(F)(F)F)nc1]

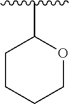 [SMILES: *C1CCCCO1]

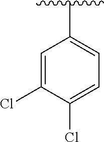 [SMILES: *c1ccc(Cl)c(Cl)c1]

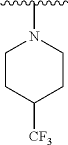 [SMILES: *N1CCC(C(F)(F)F)CC1]

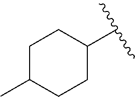 [SMILES: *C1CCC(C)CC1]

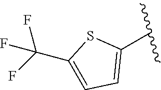 [SMILES: *c1ccc(C(F)(F)F)s1]

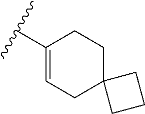 [SMILES: *C1=CCC2(CCC2)CC1]

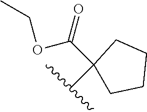 [SMILES: *C1(C(=O)OCC)CCCC1]

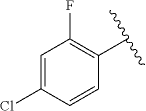 [SMILES: *c1ccc(Cl)cc1F]

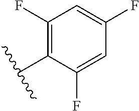 [SMILES: *c1c(F)cc(F)cc1F]

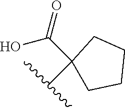 [SMILES: *C1(C(=O)O)CCCC1]

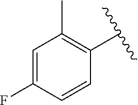 [SMILES: *c1ccc(F)cc1C]

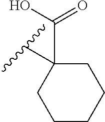 [SMILES: *C1(C(=O)O)CCCCC1]

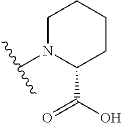 [SMILES: *N1CCCC[C@@H]1C(=O)O]

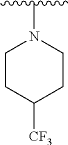 [SMILES: *N1CCC(C(F)(F)F)CC1]

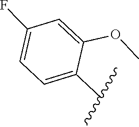 [SMILES: *c1ccc(F)cc1OC]

—C(CH₃)₃

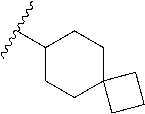 [SMILES: *C1CCC2(CCC2)CC1]

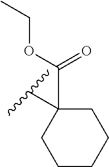 [SMILES: *C1(C(=O)OCC)CCCCC1]

As defined generally above, L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—

In some embodiments, L² is a covalent bond. In some embodiments, L² is a saturated or unsaturated, straight or branched, optionally substituted bivalent C₁₋₄ hydrocarbon chain, wherein 0-2 methylene units of L² are independently replaced by —C(O)O—, —C(O)—, or —C(O)NR—. In some embodiments, L² is a C₁₋₄ alkylene chain, wherein 1-2 methylene units of L² are independently replaced by —C(O)O—, —C(O)—, or —C(O)NR—. In some embodiments, L² is C₁₋₄ alkylene chain, wherein 1 methylene unit of L² is replaced by —C(O)O—, —C(O)—, or —C(O)NR—. In some embodiments, L² is a saturated optionally substituted bivalent C₁₋₄ hydrocarbon chain. In some embodiments, L² is a saturated bivalent C₁₋₄ hydrocarbon chain, substituted on a single methylene unit by two substituents, which together with the intervening carbon atom of the methylene unit form a 3-7 membered carbocyclic ring or heterocyclic ring (having 1-2 heteroatoms independently selected from nitrogen, oxygen, and sulfur). In some embodiments, L² is

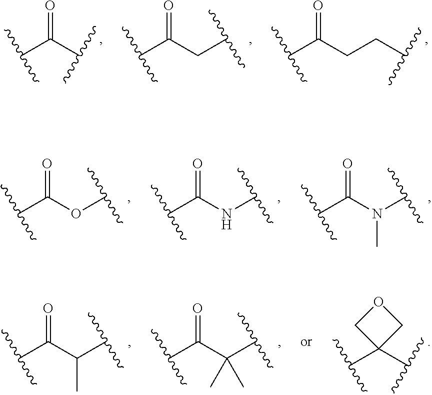

In some embodiments, L² is

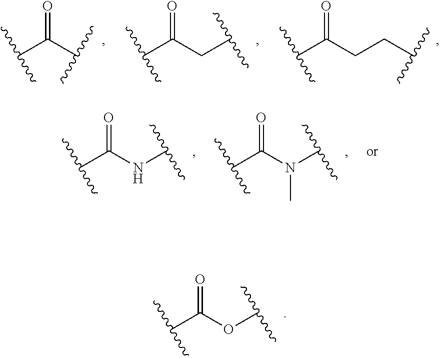

In some embodiments, L² is

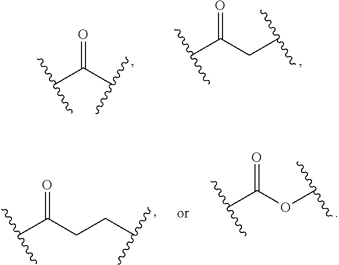

In some embodiments, L² is

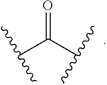 [SMILES: *C(*)=O]

In some embodiments, L² is a saturated, straight or branched, optionally substituted bivalent C₁₋₄ hydrocarbon chain. In some embodiments, L² is methylene. In some embodiments, L² is —S(O)₂—. In some embodiments, L² is selected from those depicted in the compounds of Table 8A, below. In some embodiments, L² is selected from those depicted in the compounds of Table 8B, below.

As defined generally above, R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷.

Also as defined generally above, R⁶ is an optionally substituted C₁₋₆ aliphatic group, or a cyclic group selected from a 3-8 membered saturated or partially unsaturated monocyclic carbocyclic ring, a 5-12 membered saturated or partially unsaturated bicyclic carbocyclic ring that is optionally bridged, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with x instances of R⁷.

In some embodiments, R⁶ is an optionally substituted C₁₋₆ aliphatic group. In some embodiments, R⁶ is an optionally substituted methyl, ethyl, isopropyl, or tert-butyl group.

In some embodiments, R⁶ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷. In some embodiments, R⁶ is a 3-8 membered saturated or partially unsaturated monocyclic carbocyclic ring, optionally substituted with one or more instances of R⁷. In some embodiments, R⁶ is a phenyl group, optionally substituted with one or more instances of R⁷. In some embodiments, R⁶ is a cyclic group selected from cyclopropyl, cyclobutyl, cyclohexyl and phenyl, wherein the cyclic group is optionally substituted with one or more instances of R⁷. In some embodiments, R⁶ is a cyclopropyl group, optionally substituted with one or more instances of R⁷. In some embodiments, R⁶ is selected from those depicted in the compounds of Table 8A, below. In some embodiments, R⁶ is selected from those depicted in the compounds of Table 8B, below.

In some embodiments, R⁶ is a cyclic group substituted with 1 instance of R⁷. In some embodiments, R⁶ is a cyclic group substituted with 2 instances of R⁷. In some embodiments, R⁶ is a cyclic group substituted with 3 instances of R⁷. In some embodiments, R⁶ is a cyclic group substituted with 4 instances of R⁷. In some embodiments, R⁶ is a cyclic group substituted with 5 instances of R⁷.

In some embodiments, -L²-R⁶ is a substituent of Table 4 or Table 5. In some embodiments, -L²-R⁶ or R⁶ is a substituent of Table 5. Also contemplated are embodiments wherein the -L²-R¹ of Table 4 or Table 5 is further substituted with one or more instances of R⁷ on R⁶ which are not shown in Table 4 or Table 5. In some embodiments, -L²-R⁶ is selected from those depicted in the compounds of Table 8A, below. In some embodiments, -L^{Z}-R⁶ is selected from those depicted in the compounds of Table 8B, below.

TABLE 4
Exemplary —L²—R⁶ substituents

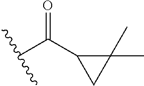 [SMILES: *C(=O)C1CC1(C)C]

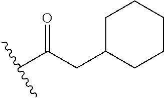 [SMILES: *C(=O)CC1CCCCC1]

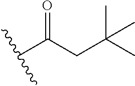 [SMILES: *C(=O)CC(C)(C)C]

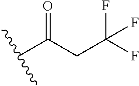 [SMILES: *C(=O)CC(F)(F)F]

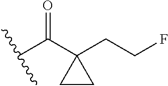 [SMILES: *C(=O)C1(CCF)CC1]

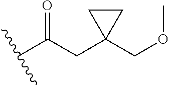 [SMILES: *C(=O)CC1(COC)CC1]

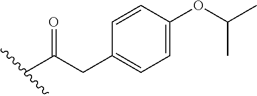 [SMILES: *C(=O)Cc1ccc(OC(C)C)cc1]

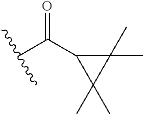 [SMILES: *C(=O)C1C(C)(C)C1(C)C]

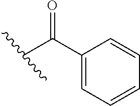 [SMILES: *C(=O)c1ccccc1]

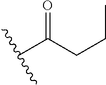 [SMILES: *C(=O)CCC]

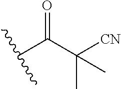 [SMILES: *C(=O)C(C)(C)C#N]

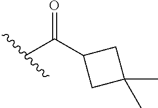 [SMILES: *C(=O)C1CC(C)(C)C1]

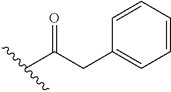 [SMILES: *C(=O)Cc1ccccc1]

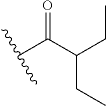 [SMILES: *C(=O)C(CC)CC]

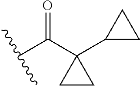 [SMILES: *C(=O)C1(C2CC2)CC1]

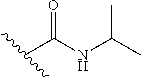 [SMILES: *C(=O)NC(C)C]

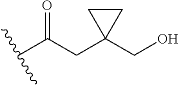 [SMILES: *C(=O)CC1(CO)CC1]

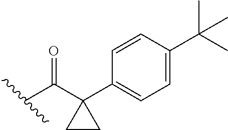 [SMILES: *C(=O)C1(c2ccc(C(C)(C)C)cc2)CC1]

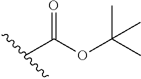 [SMILES: *C(=O)OC(C)(C)C]

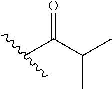 [SMILES: *C(=O)C(C)C]

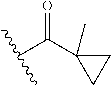 [SMILES: *C(=O)C1(C)CC1]

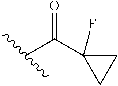 [SMILES: *C(=O)C1(F)CC1]

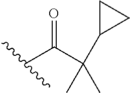 [SMILES: *C(=O)C(C)(C)C1CC1]

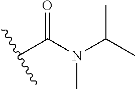 [SMILES: *C(=O)N(C)C(C)C]

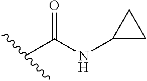 [SMILES: *C(=O)NC1CC1]

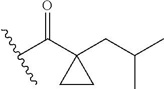 [SMILES: *C(=O)C1(CC(C)C)CC1]

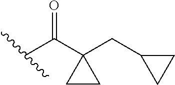 [SMILES: *C(=O)C1(CC2CC2)CC1]

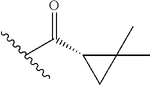 [SMILES: *C(=O)[C@H]1CC1(C)C]

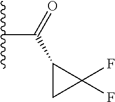 [SMILES: *C(=O)[C@H]1CC1(F)F]

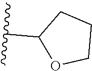 [SMILES: *C1CCCO1]

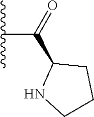 [SMILES: *C(=O)[C@H]1CCCN1]

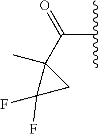 [SMILES: *C(=O)C1(C)CC1(F)F]

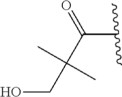 [SMILES: *C(=O)C(C)(C)CO]

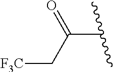 [SMILES: *C(=O)CC(F)(F)F]

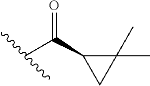 [SMILES: *C(=O)[C@@H]1CC1(C)C]

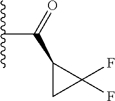 [SMILES: *C(=O)[C@@H]1CC1(F)F]

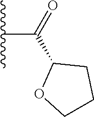 [SMILES: *C(=O)[C@@H]1CCCO1]

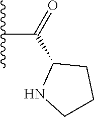 [SMILES: *C(=O)[C@@H]1CCCN1]

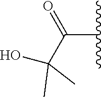 [SMILES: *C(=O)C(C)(C)O]

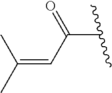 [SMILES: *C(=O)C=C(C)C]

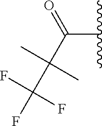 [SMILES: *C(=O)C(C)(C)C(F)(F)F]

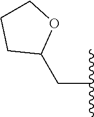 [SMILES: *CC1CCCO1]

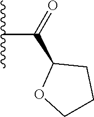 [SMILES: *C(=O)[C@H]1CCCO1]

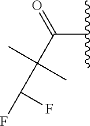 [SMILES: *C(=O)C(C)(C)C(F)F]

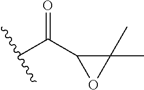 [SMILES: *C(=O)C1OC1(C)C]

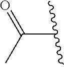 [SMILES: *C(C)=O]

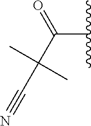 [SMILES: *C(=O)C(C)(C)C#N]

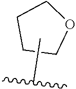

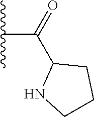 [SMILES: *C(=O)C1CCCN1]

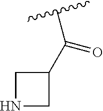 [SMILES: *C(=O)C1CNC1]

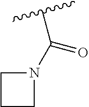 [SMILES: *C(=O)N1CCC1]

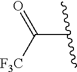 [SMILES: *C(=O)C(F)(F)F]

TABLE 5
Exemplary —L²—R⁶
or R⁶ substituents

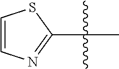 [SMILES: *c1nccs1]

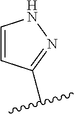 [SMILES: *c1cc[nH]n1]

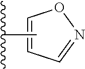

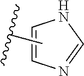

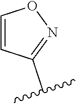 [SMILES: *c1ccon1]

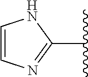 [SMILES: *c1ncc[nH]1]

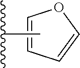

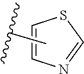

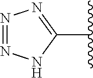 [SMILES: *c1nnn[nH]1]

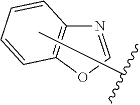

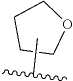

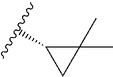 [SMILES: *[C@H]1CC1(C)C]

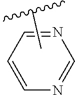

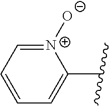 [SMILES: *c1cccc[n+]1[O-]]

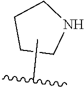

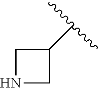 [SMILES: *C1CNC1]

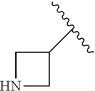 [SMILES: *C1CNC1]

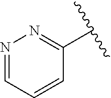 [SMILES: *c1cccnn1]

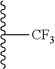 [SMILES: *C(F)(F)F]

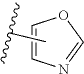

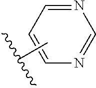

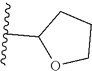 [SMILES: *C1CCCO1]

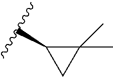 [SMILES: *[C@@H]1CC1(C)C]

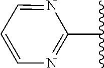 [SMILES: *c1ncccn1]

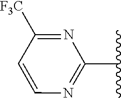 [SMILES: *c1nccc(C(F)(F)F)n1]

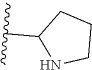 [SMILES: *C1CCCN1]

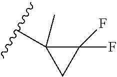 [SMILES: *C1(C)CC1(F)F]

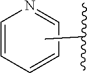

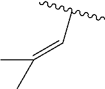 [SMILES: *C=C(C)C]

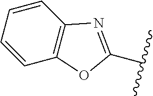 [SMILES: *c1nc2ccccc2o1]

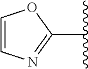 [SMILES: *c1ncco1]

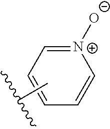

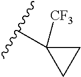 [SMILES: *C1(C(F)(F)F)CC1]

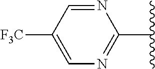 [SMILES: *c1ncc(C(F)(F)F)cn1]

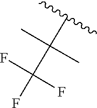 [SMILES: *C(C)(C)C(F)(F)F]

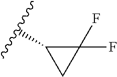 [SMILES: *[C@H]1CC1(F)F]

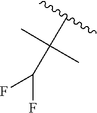 [SMILES: *C(C)(C)C(F)F]

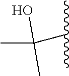 [SMILES: *C(C)(C)O]

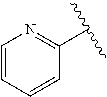 [SMILES: *c1ccccn1]

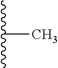

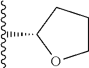 [SMILES: *[C@@H]1CCCO1]

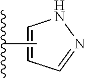

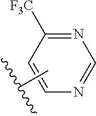

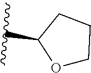 [SMILES: *[C@H]1CCCO1]

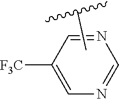

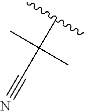 [SMILES: *C(C)(C)C#N]

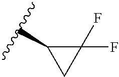 [SMILES: *[C@@H]1CC1(F)F]

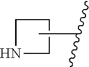

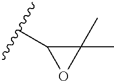 [SMILES: *C1OC1(C)C]

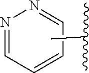

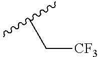 [SMILES: *CC(F)(F)F]

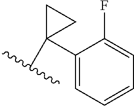 [SMILES: *C1(c2ccccc2F)CC1]

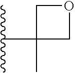 [SMILES: *C1(C)COC1]

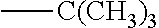

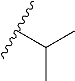 [SMILES: *C(C)C]

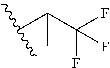 [SMILES: *C(C)C(F)(F)F]

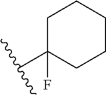 [SMILES: *C1(F)CCCCC1]

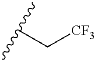 [SMILES: *CC(F)(F)F]

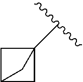 [SMILES: *C12CC(C1)C2]

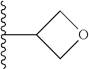 [SMILES: *C1COC1]

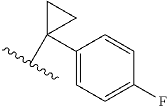 [SMILES: *C1(c2ccc(F)cc2)CC1]

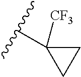 [SMILES: *C1(C(F)(F)F)CC1]

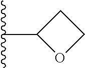 [SMILES: *C1CCO1]

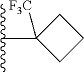 [SMILES: *C1(C(F)(F)F)CCC1]

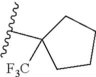 [SMILES: *C1(C(F)(F)F)CCCC1]

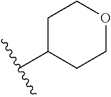 [SMILES: *C1CCOCC1]

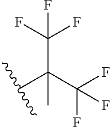 [SMILES: *C(C)(C(F)(F)F)C(F)(F)F]

In some embodiments, -L²-R⁶ is

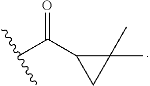 [SMILES: *C(=O)C1CC1(C)C]

In some embodiments, -L²-R⁶ is

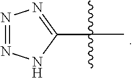 [SMILES: *c1nnn[nH]1]

In some embodiments, -L²-R⁶ is

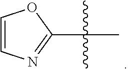 [SMILES: *c1ncco1]

In some embodiments, -L²-R⁶ is

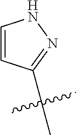 [SMILES: *c1cc[nH]n1]

As defined generally above, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, each instance of R⁷ is independently halogen, —OR, —CN, an optionally substituted C₁₋₆ aliphatic group, an optionally substituted C₁₋₆ aliphatic-Cy group, or Cy. In some embodiments, each instance of R⁷ is independently —F, methyl, ethyl, isopropyl, isobutyl, —CN, optionally substituted phenyl, optionally substituted benzyl, —CF₃, —CH₂OH, —CH₂OCH₃, —CH₂CH₂OCH₃, —CH₂CH₂F, cyclopropyl or —CH₂-(cyclopropyl). In some embodiments, each instance of R⁷ is independently a C₁₋₆ aliphatic group.

In some embodiments, there are 0 instances of R⁷. In some embodiments, there is 1 instance of R⁷. In some embodiments, there are 2 instances of R⁷. In some embodiments, there are 3 instances of R⁷. In some embodiments, there are 4 instances of R⁷.

As defined generally above, L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—

In some embodiments, L³ is a covalent bond. In some embodiments, L³ is a saturated or unsaturated, straight or branched, optionally substituted bivalent C₁₋₄ hydrocarbon chain, wherein 0-2 methylene units of L³ are independently replaced by —S(O)₂—, —C(O)NR—, or —C(O)—. In some embodiments, L³ is a C₁₋₄ alkylene chain, wherein 1-2 methylene units of L³ are independently replaced by —S(O)₂—, —C(O)NR—, or —C(O)—. In some embodiments, L³ is C₁₋₄ alkylene chain, wherein 1 methylene unit of L³ is replaced by —S(O)₂—, —C(O)NR—, or —C(O)—. In some embodiments, L³ is a saturated or unsaturated, straight or branched, optionally substituted bivalent C₁₋₄ alkylene chain, wherein 0-2 methylene units of L³ are independently replaced by —C(O)O— or —C(O)—. In some embodiments, L³ is a C₁₋₄ alkylene chain, wherein 1-2 methylene units of L³ are independently replaced by —C(O)O— or —C(O)—. In some embodiments, L³ is C₁₋₄ alkylene chain, wherein 1 methylene unit of L³ is replaced by —C(O)O— or —C(O)—. In some embodiments, L³ is a saturated optionally substituted bivalent C₁₋₄ hydrocarbon chain. In some embodiments, L³ is a saturated bivalent C₁₋₄ hydrocarbon chain, substituted on a single methylene unit by two substituents, which together with the intervening carbon atom (the single methylene unit) form a 3-7 membered carbocyclic ring or heterocyclic ring (having 1-2 heteroatoms independently selected from nitrogen, oxygen, and sulfur). In some embodiments, L³ is

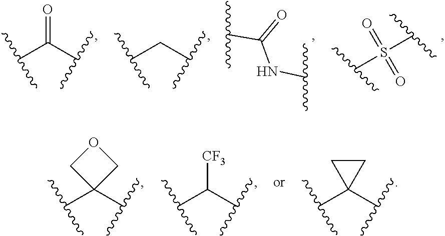

In some embodiments, L³ is

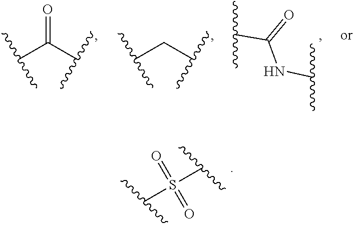

In some embodiments, L³ is

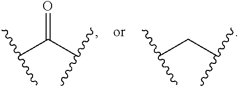

In some embodiments, L³ is selected from those depicted in the compounds of Table 8A, below. In some embodiments, L³ is selected from those depicted in the compounds of Table 8B, below.

As defined generally above, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a cyclic group selected from a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a 3-8 membered saturated or partially unsaturated monocyclic heterocyclic ring (having 1-2 heteroatoms independently selected from nitrogen, oxygen, and sulfur), optionally substituted with one or more instances of R⁹. In some embodiments, R⁸ is a 5-6 membered monocyclic heteroaromatic ring (having 1-4 heteroatoms independently selected from nitrogen, oxygen, and sulfur), optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R⁹. In some embodiments, R⁸ is a cyclic group selected from pyrazolyl, oxazolyl, thiazolyl, pyrrolidinyl, tetrahydropyranyl, pyridinyl, imidazolyl, indolyl, 1,2,4-triazolyl, 1,2,4-thiadiazolyl, piperidinyl, pyrazinyl, phenyl, tetrahydrofuranyl, benzo[d]thiazolyl, thiazolo[4,5-d]pyrimidinyl, and indazolyl, wherein the cyclic group is optionally substituted with one or more instances of R⁹. In some embodiments, R⁸ is a pyrazolyl or thiazolyl group, optionally substituted with one or more instances of R⁹. In some embodiments, R⁸ is a pyrazolyl or thiazolyl group.

In some embodiments, R⁸ is a cyclic group selected from an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R⁹, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, the R⁸ is a cyclic group substituted with 1 instance of R⁹. In some embodiments, the R⁸ is a cyclic group substituted with 2 instances of R⁹. In some embodiments, the R⁸ is a cyclic group substituted with 3 instances of R⁹. In some embodiments, the R⁸ is a cyclic group substituted with 4 instances of R⁹. In some embodiments, the R⁸ is a cyclic group substituted with 5 instances of R⁹.

In some embodiments, L³ is a covalent bond and R⁸ is a 5-6 membered heteroaryl or an 8-10 membered bicyclic heteroaromatic ring (having 1-5 heteroatoms independently selected from nitrogen, oxygen, and sulfur), optionally substituted with one or more instances of R⁹.

In some embodiments, R⁸ is selected from those depicted in the compounds of Table 8A, below. In some embodiments, R⁸ is selected from those depicted in the compounds of Table 8B, below.

As defined generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, there are 0 instances of R⁹. In some embodiments, there is 1 instance of R⁹. In some embodiments, there are 2 instances of R⁹. In some embodiments, there are 3 instances of R⁹.

Also in aspects described generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —C(O)N(R)S(O)₂R, —C(O)N(R)S(O)₂NR₂, an optionally substituted C₁₋₆ aliphatic group, an optionally substituted C₁₋₆ aliphatic-Cy group, or Cy.

In some embodiments, each instance of R⁹ is independently halogen, an optionally substituted C₁₋₆ aliphatic group, an optionally substituted C₁₋₆ aliphatic-Cy group, or Cy. In some embodiments, each instance of R⁹ is independently an optionally substituted C₁₋₆ aliphatic-Cy group, wherein the Cy is an optionally substituted group selected from phenyl, cyclohexyl, pyridinyl, piperidinyl, cyclopropyl, or tetrahydropyranyl. In some embodiments, R⁹ is a benzylic group. In some embodiments, each instance of R⁹ is independently halogen or an optionally substituted C₁₋₆ aliphatic group. In some embodiments, R⁹ is selected from those depicted in the compounds of Table 8A, below. In some embodiments, R⁹ is selected from those depicted in the compounds of Table 8B, below.

In some embodiments, -L³-R⁸ is a substituent of Table 6 or Table 7, wherein R⁸ is optionally substituted with one or more instances of R⁹. In some embodiments, the -L³-R⁸ of Table 6 or Table 7 is shown with the one or more instance of R⁹. Also contemplated are embodiments wherein the -L³-R⁸ of Table 6 or Table 7 is further substituted with one or more instances of R⁹ which are not shown in Table 6 or Table 7.

In some embodiments, -L³-R⁸ is a substituent of Table 6 or Table 7, wherein R⁸ is optionally substituted with one or more instances of R⁹, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—

In some embodiments, the -L³-R⁸ of Table 6 or Table 7 is shown with the one or more instance of R⁹, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

Also contemplated are embodiments wherein the -L³-R⁸ of Table 6 or Table 7 is further substituted with one or more instances of R⁹ which are not shown in Table 6 or Table 7, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

TABLE 6
Exemplary —L³—R⁸ substituents

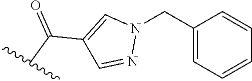 [SMILES: *C(=O)c1cnn(Cc2ccccc2)c1]

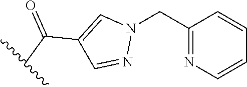 [SMILES: *C(=O)c1cnn(Cc2ccccn2)c1]

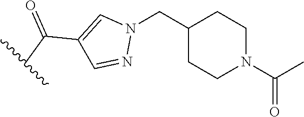 [SMILES: *C(=O)c1cnn(CC2CCN(C(C)=O)CC2)c1]

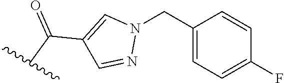 [SMILES: *C(=O)c1cnn(Cc2ccc(F)cc2)c1]

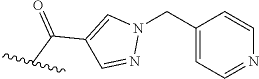 [SMILES: *C(=O)c1cnn(Cc2ccncc2)c1]

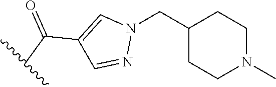 [SMILES: *C(=O)c1cnn(CC2CCN(C)CC2)c1]

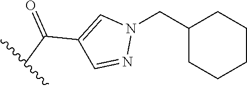 [SMILES: *C(=O)c1cnn(CC2CCCCC2)c1]

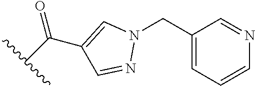 [SMILES: *C(=O)c1cnn(Cc2cccnc2)c1]

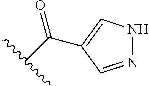 [SMILES: *C(=O)c1cn[nH]c1]

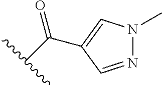 [SMILES: *C(=O)c1cnn(C)c1]

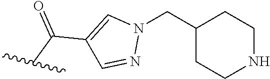 [SMILES: *C(=O)c1cnn(CC2CCNCC2)c1]

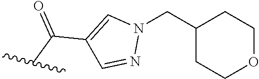 [SMILES: *C(=O)c1cnn(CC2CCOCC2)c1]

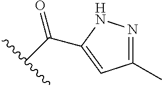 [SMILES: *C(=O)c1cc(C)n[nH]1]

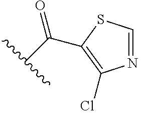 [SMILES: *C(=O)c1scnc1Cl]

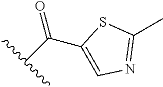 [SMILES: *C(=O)c1cnc(C)s1]

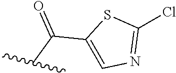 [SMILES: *C(=O)c1cnc(Cl)s1]

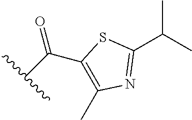 [SMILES: *C(=O)c1sc(C(C)C)nc1C]

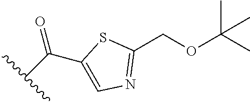 [SMILES: *C(=O)c1cnc(COC(C)(C)C)s1]

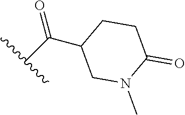 [SMILES: *C(=O)C1CCC(=O)N(C)C1]

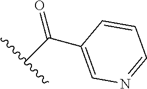 [SMILES: *C(=O)c1cccnc1]

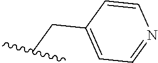 [SMILES: *Cc1ccncc1]

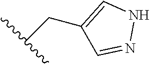 [SMILES: *Cc1cn[nH]c1]

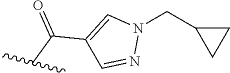 [SMILES: *C(=O)c1cnn(CC2CC2)c1]

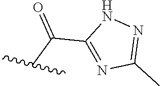 [SMILES: *C(=O)c1nc(C)n[nH]1]

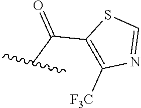 [SMILES: *C(=O)c1scnc1C(F)(F)F]

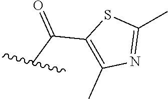 [SMILES: *C(=O)c1sc(C)nc1C]

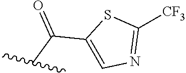 [SMILES: *C(=O)c1cnc(C(F)(F)F)s1]

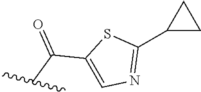 [SMILES: *C(=O)c1cnc(C2CC2)s1]

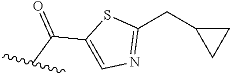 [SMILES: *C(=O)c1cnc(CC2CC2)s1]

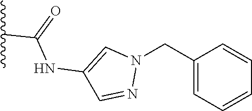 [SMILES: *C(=O)Nc1cnn(Cc2ccccc2)c1]

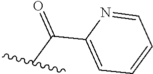 [SMILES: *C(=O)c1ccccn1]

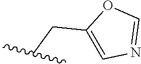 [SMILES: *Cc1cnco1]

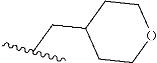 [SMILES: *CC1CCOCC1]

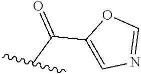 [SMILES: *C(=O)c1cnco1]

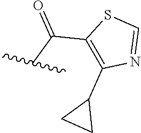 [SMILES: *C(=O)c1scnc1C1CC1]

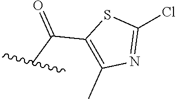 [SMILES: *C(=O)c1sc(Cl)nc1C]

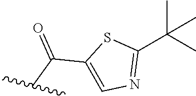 [SMILES: *C(=O)c1cnc(C(C)(C)C)s1]

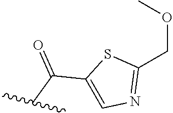 [SMILES: *C(=O)c1cnc(COC)s1]

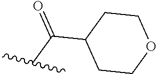 [SMILES: *C(=O)C1CCOCC1]

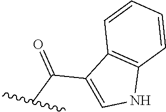 [SMILES: *C(=O)c1c[nH]c2ccccc12]

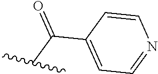 [SMILES: *C(=O)c1ccncc1]

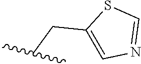 [SMILES: *Cc1cncs1]

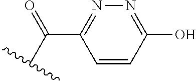 [SMILES: *C(=O)c1ccc(O)nn1]

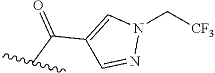 [SMILES: *C(=O)c1cnn(CC(F)(F)F)c1]

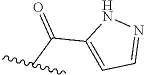 [SMILES: *C(=O)c1ccn[nH]1]

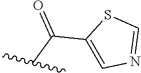 [SMILES: *C(=O)c1cncs1]

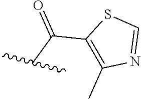 [SMILES: *C(=O)c1scnc1C]

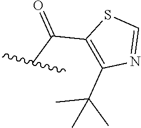 [SMILES: *C(=O)c1scnc1C(C)(C)C]

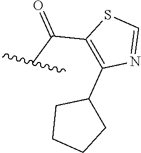 [SMILES: *C(=O)c1scnc1C1CCCC1]

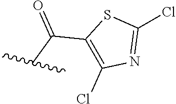 [SMILES: *C(=O)c1sc(Cl)nc1Cl]

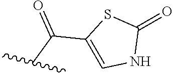 [SMILES: *C(=O)c1c[nH]c(=O)s1]

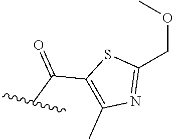 [SMILES: *C(=O)c1sc(COC)nc1C]

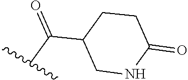 [SMILES: *C(=O)C1CCC(=O)NC1]

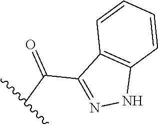 [SMILES: *C(=O)c1n[nH]c2ccccc12]

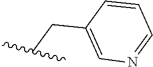 [SMILES: *Cc1cccnc1]

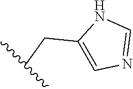 [SMILES: *Cc1cnc[nH]1]

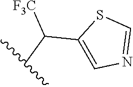 [SMILES: *C(c1cncs1)C(F)(F)F]

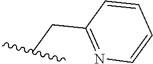 [SMILES: *Cc1ccccn1]

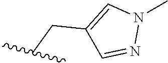 [SMILES: *Cc1cnn(C)c1]

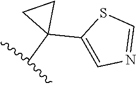 [SMILES: *C1(c2cncs2)CC1]

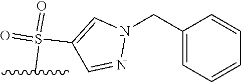 [SMILES: *S(=O)(=O)c1cnn(Cc2ccccc2)c1]

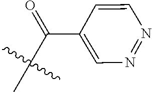 [SMILES: *C(=O)c1ccnnc1]

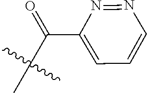 [SMILES: *C(=O)c1cccnn1]

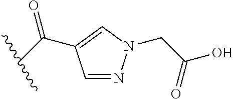 [SMILES: *C(=O)c1cnn(CC(=O)O)c1]

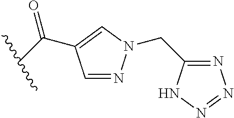 [SMILES: *C(=O)c1cnn(Cc2nnn[nH]2)c1]

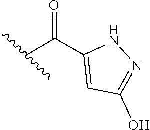 [SMILES: *C(=O)c1cc(O)n[nH]1]

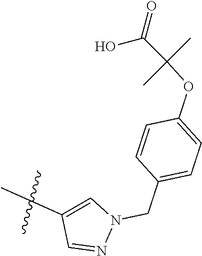 [SMILES: *c1cnn(Cc2ccc(OC(C)(C)C(=O)O)cc2)c1]

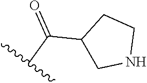 [SMILES: *C(=O)C1CCNC1]

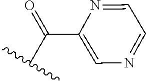 [SMILES: *C(=O)c1cnccn1]

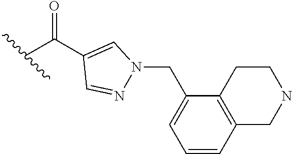 [SMILES: *C(=O)c1cnn(Cc2cccc3c2CCNC3)c1]

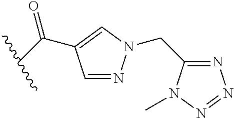 [SMILES: *C(=O)c1cnn(Cc2nnnn2C)c1]

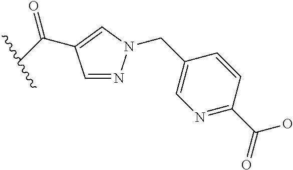 [SMILES: *C(=O)c1cnn(Cc2ccc(C(=O)O)nc2)c1]

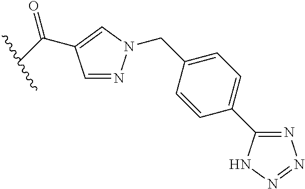 [SMILES: *C(=O)c1cnn(Cc2ccc(-c3nnn[nH]3)cc2)c1]

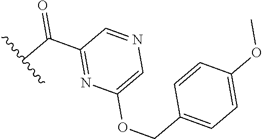 [SMILES: *C(=O)c1cncc(OCc2ccc(OC)cc2)n1]

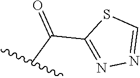 [SMILES: *C(=O)c1nncs1]

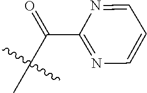 [SMILES: *C(=O)c1ncccn1]

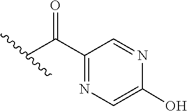 [SMILES: *C(=O)c1cnc(O)cn1]

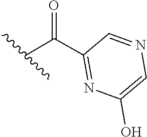 [SMILES: *C(=O)c1cncc(O)n1]

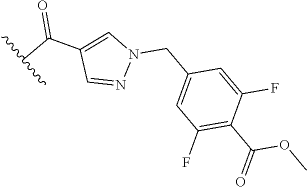 [SMILES: *C(=O)c1cnn(Cc2cc(F)c(C(=O)OC)c(F)c2)c1]

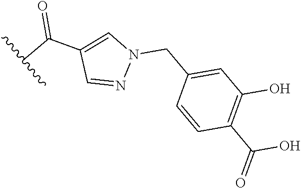 [SMILES: *C(=O)c1cnn(Cc2ccc(C(=O)O)c(O)c2)c1]

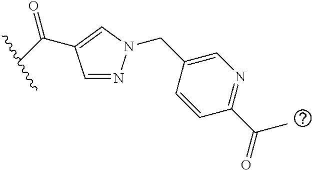 [SMILES: *C(=O)c1cnn(Cc2ccc(C(C)=O)nc2)c1]

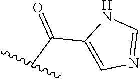 [SMILES: *C(=O)c1cnc[nH]1]

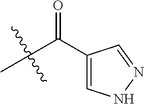 [SMILES: *C(=O)c1cn[nH]c1]

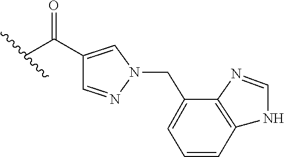 [SMILES: *C(=O)c1cnn(Cc2cccc3[nH]cnc23)c1]

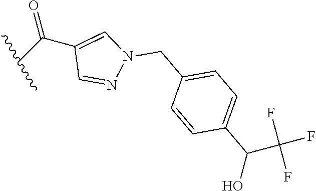 [SMILES: *C(=O)c1cnn(Cc2ccc(C(O)C(F)(F)F)cc2)c1]

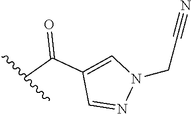 [SMILES: *C(=O)c1cnn(CC#N)c1]

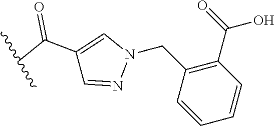 [SMILES: *C(=O)c1cnn(Cc2ccccc2C(=O)O)c1]

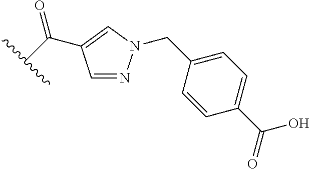 [SMILES: *C(=O)c1cnn(Cc2ccc(C(=O)O)cc2)c1]

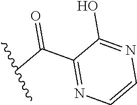 [SMILES: *C(=O)c1nccnc1O]

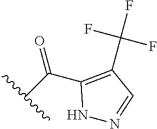 [SMILES: *C(=O)c1[nH]ncc1C(F)(F)F]

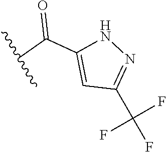 [SMILES: *C(=O)c1cc(C(F)(F)F)n[nH]1]

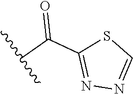 [SMILES: *C(=O)c1nncs1]

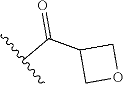 [SMILES: *C(=O)C1COC1]

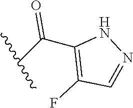 [SMILES: *C(=O)c1[nH]ncc1F]

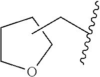

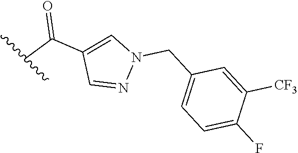 [SMILES: *C(=O)c1cnn(Cc2ccc(F)c(C(F)(F)F)c2)c1]

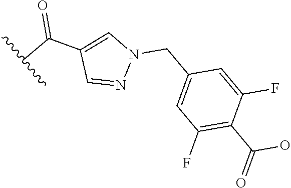 [SMILES: *C(=O)c1cnn(Cc2cc(F)c(C(=O)O)c(F)c2)c1]

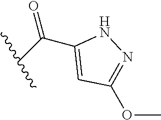 [SMILES: *C(=O)c1cc(OC)n[nH]1]

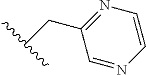 [SMILES: *Cc1cnccn1]

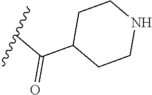 [SMILES: *C(=O)C1CCNCC1]

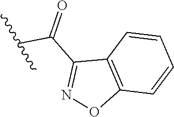 [SMILES: *C(=O)c1noc2ccccc12]

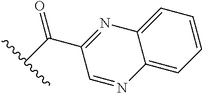 [SMILES: *C(=O)c1cnc2ccccc2n1]

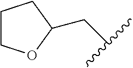 [SMILES: *CC1CCCO1]

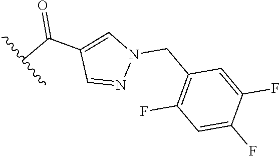 [SMILES: *C(=O)c1cnn(Cc2cc(F)c(F)cc2F)c1]

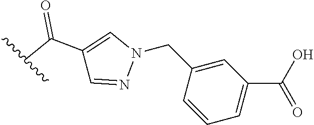 [SMILES: *C(=O)c1cnn(Cc2cccc(C(=O)O)c2)c1]

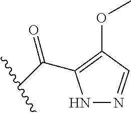 [SMILES: *C(=O)c1[nH]ncc1OC]

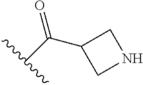 [SMILES: *C(=O)C1CNC1]

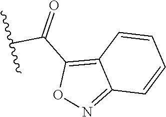 [SMILES: *C(=O)c1onc2ccccc12]

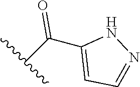 [SMILES: *C(=O)c1ccn[nH]1]

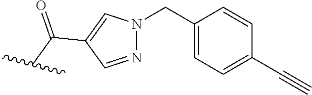 [SMILES: *C(=O)c1cnn(Cc2ccc(C#C)cc2)c1]

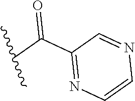 [SMILES: *C(=O)c1cnccn1]

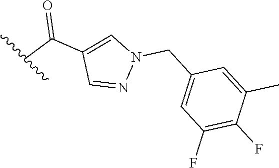 [SMILES: *C(=O)c1cnn(Cc2cc(C)c(F)c(F)c2)c1]

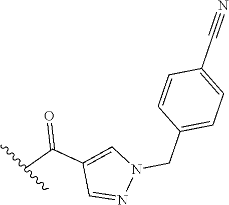 [SMILES: *C(=O)c1cnn(Cc2ccc(C#N)cc2)c1]

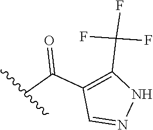 [SMILES: *C(=O)c1cn[nH]c1C(F)(F)F]

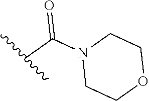 [SMILES: *C(=O)N1CCOCC1]

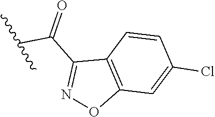 [SMILES: *C(=O)c1noc2cc(Cl)ccc12]

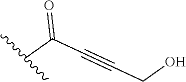 [SMILES: *C(=O)C#CCO]

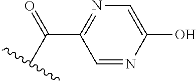 [SMILES: *C(=O)c1cnc(O)cn1]

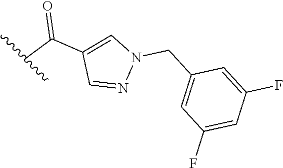 [SMILES: *C(=O)c1cnn(Cc2cc(F)cc(F)c2)c1]

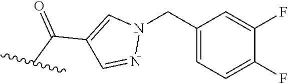 [SMILES: *C(=O)c1cnn(Cc2ccc(F)c(F)c2)c1]

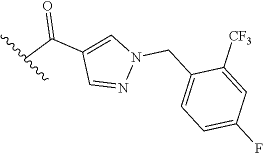 [SMILES: *C(=O)c1cnn(Cc2ccc(F)cc2C(F)(F)F)c1]

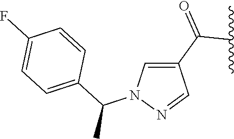 [SMILES: *C(=O)c1cnn([C@@H](C)c2ccc(F)cc2)c1]

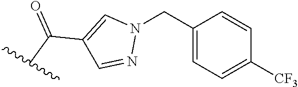 [SMILES: *C(=O)c1cnn(Cc2ccc(C(F)(F)F)cc2)c1]

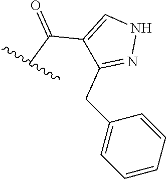 [SMILES: *C(=O)c1c[nH]nc1Cc1ccccc1]

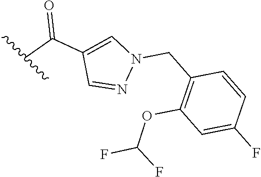 [SMILES: *C(=O)c1cnn(Cc2ccc(F)cc2OC(F)F)c1]

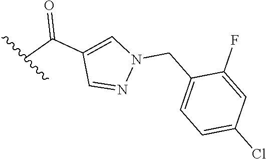 [SMILES: *C(=O)c1cnn(Cc2ccc(Cl)cc2F)c1]

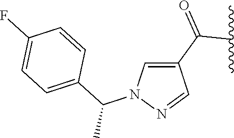 [SMILES: *C(=O)c1cnn([C@H](C)c2ccc(F)cc2)c1]

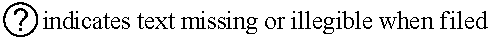

TABLE 7
Exemplary —L³—R⁸ or R⁸ substituents, wherein R⁸ is
optionally substituted with one or more instances of R⁹,
wherein the one or more R⁹ is or is not pictured in Table 7

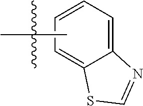

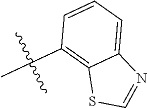 [SMILES: *c1cccc2ncsc12]

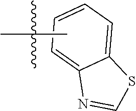

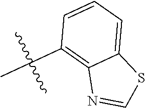 [SMILES: *c1cccc2scnc12]

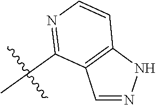 [SMILES: *c1nccc2[nH]ncc12]

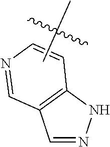

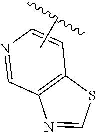

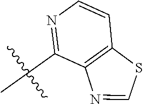 [SMILES: *c1nccc2scnc12]

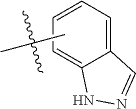

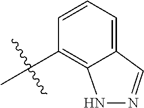 [SMILES: *c1cccc2cn[nH]c12]

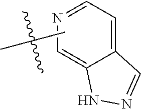

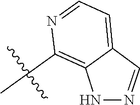 [SMILES: *c1nccc2cn[nH]c12]

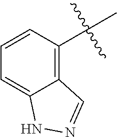 [SMILES: *c1cccc2[nH]ncc12]

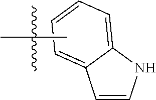

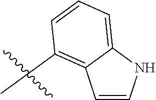 [SMILES: *c1cccc2[nH]ccc12]

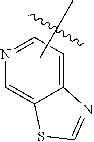

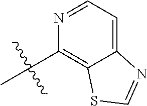 [SMILES: *c1nccc2ncsc12]

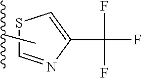

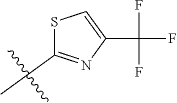 [SMILES: *c1nc(C(F)(F)F)cs1]

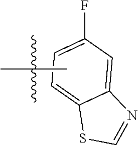

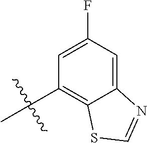 [SMILES: *c1cc(F)cc2ncsc12]

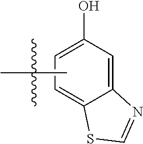

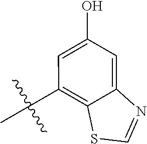 [SMILES: *c1cc(O)cc2ncsc12]

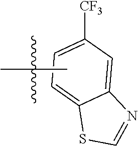

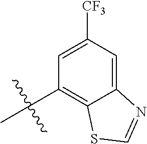 [SMILES: *c1cc(C(F)(F)F)cc2ncsc12]

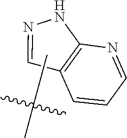

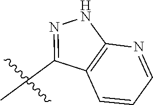 [SMILES: *c1n[nH]c2ncccc12]

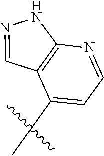 [SMILES: *c1ccnc2[nH]ncc12]

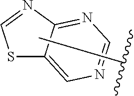

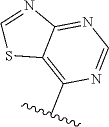 [SMILES: *c1ncnc2ncsc12]

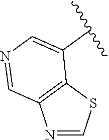 [SMILES: *c1cncc2ncsc12]

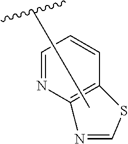

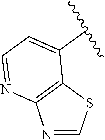 [SMILES: *c1ccnc2ncsc12]

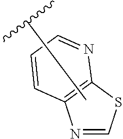

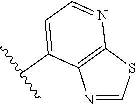 [SMILES: *c1ccnc2scnc12]

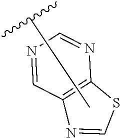

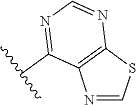 [SMILES: *c1ncnc2scnc12]

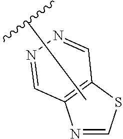

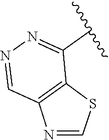 [SMILES: *c1nncc2ncsc12]

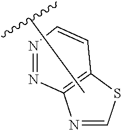

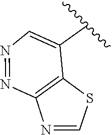 [SMILES: *c1cnnc2ncsc12]

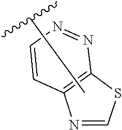

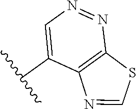 [SMILES: *c1cnnc2scnc12]

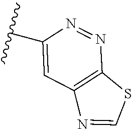 [SMILES: *c1cc2ncsc2nn1]

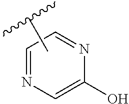

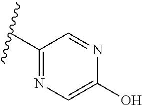 [SMILES: *c1cnc(O)cn1]

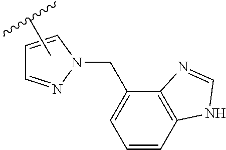

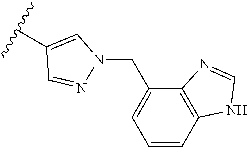 [SMILES: *c1cnn(Cc2cccc3[nH]cnc23)c1]

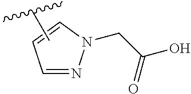

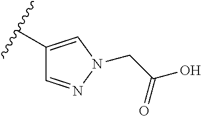 [SMILES: *c1cnn(CC(=O)O)c1]

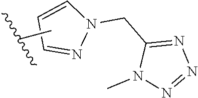

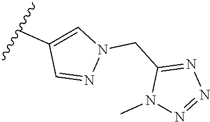 [SMILES: *c1cnn(Cc2nnnn2C)c1]

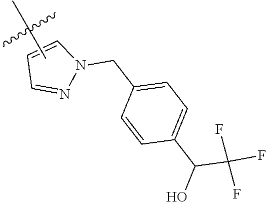

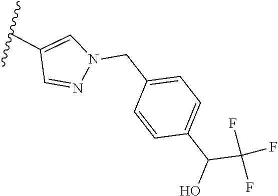 [SMILES: *c1cnn(Cc2ccc(C(O)C(F)(F)F)cc2)c1]

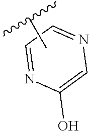

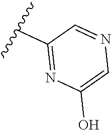 [SMILES: *c1cncc(O)n1]

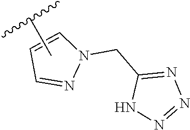

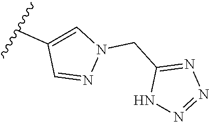 [SMILES: *c1cnn(Cc2nnn[nH]2)c1]

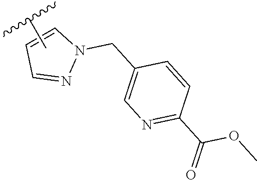

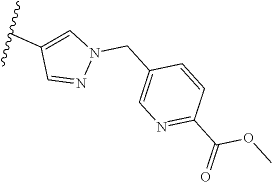 [SMILES: *c1cnn(Cc2ccc(C(=O)OC)nc2)c1]

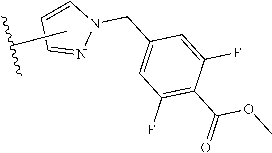

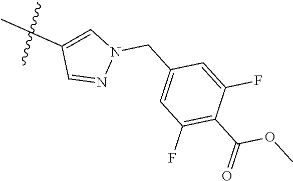 [SMILES: *c1cnn(Cc2cc(F)c(C(=O)OC)c(F)c2)c1]

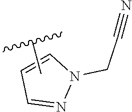

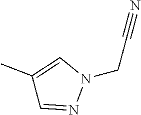

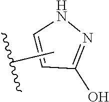

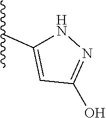 [SMILES: *c1cc(O)n[nH]1]

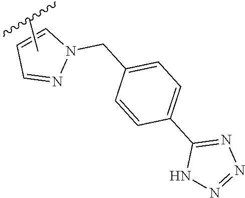

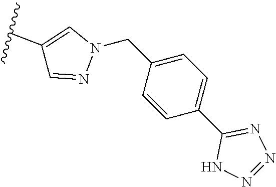 [SMILES: *c1cnn(Cc2ccc(-c3nnn[nH]3)cc2)c1]

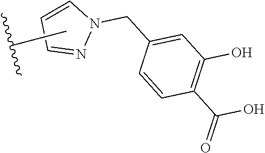

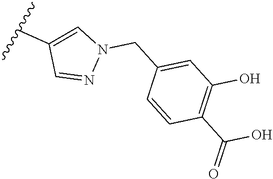 [SMILES: *c1cnn(Cc2ccc(C(=O)O)c(O)c2)c1]

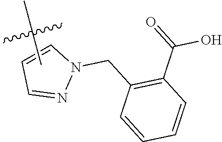

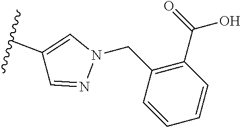 [SMILES: *c1cnn(Cc2ccccc2C(=O)O)c1]

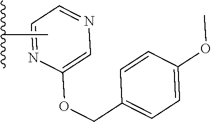

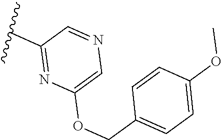 [SMILES: *c1cncc(OCc2ccc(OC)cc2)n1]

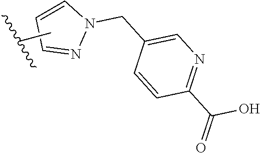

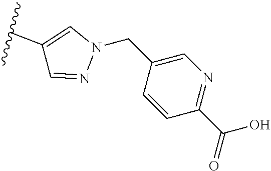 [SMILES: *c1cnn(Cc2ccc(C(=O)O)nc2)c1]

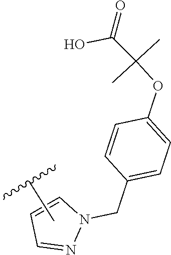

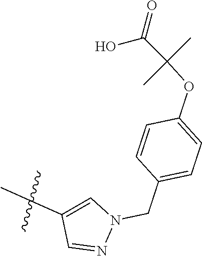 [SMILES: *c1cnn(Cc2ccc(OC(C)(C)C(=O)O)cc2)c1]

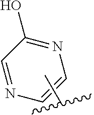

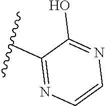 [SMILES: *c1nccnc1O]

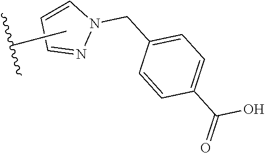

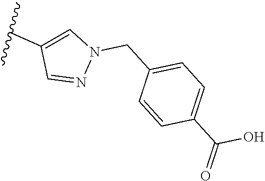 [SMILES: *c1cnn(Cc2ccc(C(=O)O)cc2)c1]

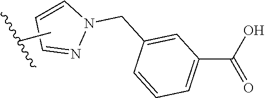

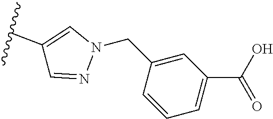 [SMILES: *c1cnn(Cc2cccc(C(=O)O)c2)c1]

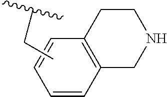

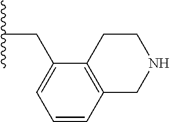 [SMILES: *Cc1cccc2c1CCNC2]

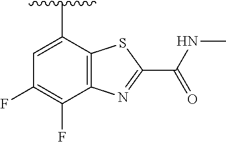 [SMILES: *c1cc(F)c(F)c2nc(C(=O)NC)sc12]

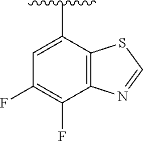 [SMILES: *c1cc(F)c(F)c2ncsc12]

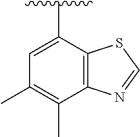 [SMILES: *c1cc(C)c(C)c2ncsc12]

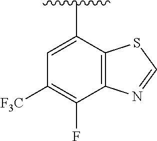 [SMILES: *c1cc(C(F)(F)F)c(F)c2ncsc12]

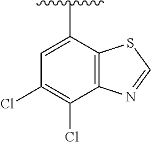 [SMILES: *c1cc(Cl)c(Cl)c2ncsc12]

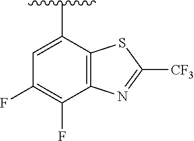 [SMILES: *c1cc(F)c(F)c2nc(C(F)(F)F)sc12]

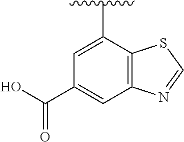 [SMILES: *c1cc(C(=O)O)cc2ncsc12]

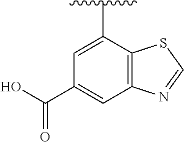 [SMILES: *c1cc(C(=O)O)cc2ncsc12]

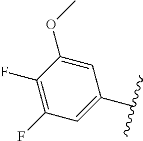 [SMILES: *c1cc(F)c(F)c(OC)c1]

In some embodiments, -L³-R⁸ is

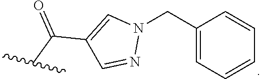 [SMILES: *C(=O)c1cnn(Cc2ccccc2)c1]

In some embodiments, -L³-R⁸ is

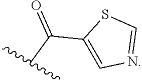 [SMILES: *C(=O)c1cncs1]

As defined generally above, each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, each Cy is independently a 3-8 membered saturated or partially unsaturated monocyclic carbocyclic ring or phenyl. In some embodiments, each Cy is independently an optionally substituted cyclic group selected from a 3-8 membered saturated or partially unsaturated monocyclic carbocyclic ring. In some embodiments, Cy is phenyl. In some embodiments, each Cy is independently a 3-8 membered saturated or partially unsaturated monocyclic heterocyclic ring (having 1-2 heteroatoms independently selected from nitrogen, oxygen, and sulfur), or a 5-6 membered monocyclic heteroaromatic ring (having 1-4 heteroatoms independently selected from nitrogen, oxygen, and sulfur). In some embodiments, each Cy is independently a 3-8 membered saturated or partially unsaturated monocyclic heterocyclic ring (having 1-2 heteroatoms independently selected from nitrogen, oxygen, and sulfur). In some embodiments, each Cy is independently a 5-6 membered monocyclic heteroaromatic ring (having 1-4 heteroatoms independently selected from nitrogen, oxygen, and sulfur).

As defined generally above, each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur), or the two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In some embodiments, R is hydrogen. In some embodiments, each R is independently an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur). In some embodiments, each R is independently an optionally substituted C₁₋₆ aliphatic group. In some embodiments, each R is independently an optionally substituted phenyl. In some embodiments, each R is independently an optionally substituted 3-7 membered saturated or partially unsaturated carbocyclic ring. In some embodiments, each R is independently an optionally substituted 3-7 membered saturated or partially unsaturated heterocyclic ring (having 1-2 heteroatoms independently selected from nitrogen, oxygen, and sulfur). In some embodiments, each R is independently an optionally substituted 5-6 membered heteroaryl ring (having 1-4 heteroatoms independently selected from nitrogen, oxygen, and sulfur).

In some embodiments, two R groups on the same nitrogen are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen are taken together with the nitrogen atom to form an optionally substituted moiety selected from the group consisting of

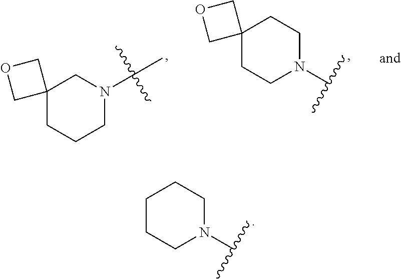 [SMILES: *N1CCCC2(COC2)C1 ; *N1CCC2(CC1)COC2 ; *N1CCCCC1]

In some embodiments, two R groups on the same nitrogen atom are taken together with the nitrogen atom to form

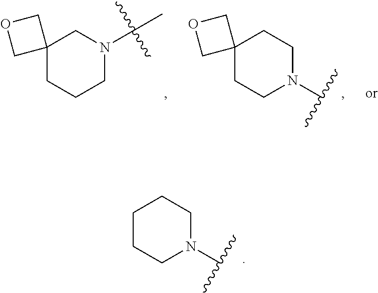 [SMILES: *N1CCCC2(COC2)C1 ; *N1CCC2(CC1)COC2 ; *N1CCCCC1]

In some embodiments, two R groups on the same nitrogen are taken together with the nitrogen atom to form optionally substituted

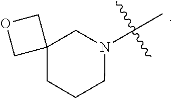 [SMILES: *N1CCCC2(COC2)C1]

In some embodiments, two R groups on the same nitrogen atom are taken together with the nitrogen atom to form

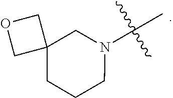 [SMILES: *N1CCCC2(COC2)C1]

In some embodiments, two R groups on the same nitrogen atom are taken together with the nitrogen atom to form optionally substituted

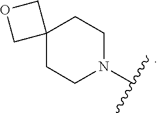 [SMILES: *N1CCC2(CC1)COC2]

In some embodiments, two R groups on the same nitrogen atom are taken together with the nitrogen atom to form

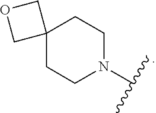 [SMILES: *N1CCC2(CC1)COC2]

In some embodiments, two R groups on the same nitrogen atom taken together with the nitrogen atom to form optionally substituted

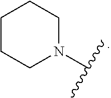 [SMILES: *N1CCCCC1]

In some embodiments, two R groups on the same nitrogen atom are taken together with the nitrogen atom to form

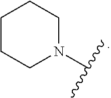 [SMILES: *N1CCCCC1]

In certain embodiments, L² is a covalent bond and L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L³ is a covalent bond and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L² and L³ are both a covalent bond. In certain embodiments, L² and L³ are both not a covalent bond.

In some embodiments, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8.

In some embodiments, y is 0. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8.

In some embodiments, z is 0. In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8.

In some embodiments, the compound of Formula I or Formula I′ is a compound of Formula IIA:

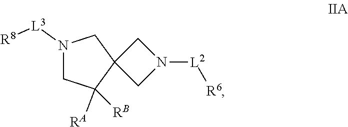

or a pharmaceutically acceptable salt thereof, wherein R^{A}, R^{B} L², R⁶, L³ and R⁸, and their constituent groups, are each as defined and described herein. In some embodiments, R^{A}, R^{B}, L², R⁶, L³ and R⁸, and their constituent groups, are each as defined and described in Formula I or Formula I′. In some embodiments, R^{A} is a substituent from Table 1. In some embodiments, -L²-R⁶ is a substituent from Table 4 or Table 5. In some embodiments, -L³-R⁸ is a substituent from Table 6 or Table 7. In some embodiments, R^{A} is a substituent from Table 1, and -L²-R⁶ is a substituent from Table 4 or Table 5. In some embodiments, R^{A} is a substituent from Table 1, and -L³-R⁸ is a substituent from Table 6 or Table 7. In some embodiments, -L²-R⁶ is a substituent from Table 4 or Table 5, and -L³-R⁸ is a substituent from Table 6 or Table 7. In some embodiments, R^{A} is a substituent from Table 1, -L²-R⁶ is a substituent from Table 4 or Table 5, and -L³-R⁸ is a substituent from Table 6 or Table 7.

In some embodiments, the compound of Formula I or Formula I′ is a compound of Formula IIB:

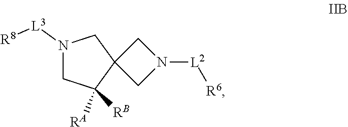

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula I or Formula I′. In some embodiments, R^{A} is a substituent from Table 1. In some embodiments, -L²-R⁶ is a substituent from Table 4 or Table 5. In some embodiments, -L³-R⁸ is a substituent from Table 6 or Table 7. In some embodiments, R^{A} is a substituent from Table 1, and -L²-R⁶ is a substituent from Table 4 or Table 5. In some embodiments, R^{A} is a substituent from Table 1, and -L³-R⁸ is a substituent from Table 6 or Table 7. In some embodiments, -L²-R⁶ is a substituent from Table 4 or Table 5, and -L³-R⁸ is a substituent from Table 6 or Table 7. In some embodiments, R^{A} is a substituent from Table 1, -L²-R⁶ is a substituent from Table 4 or Table 5, and -L³-R⁸ is a substituent from Table 6 or Table 7.

In some embodiments, the compound of Formula I or Formula I′ is a compound of Formula IIB′:

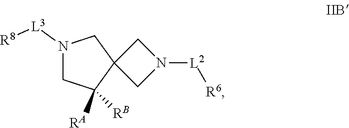

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula I or Formula I′. In some embodiments, R^{A} is a substituent from Table 1. In some embodiments, -L²-R⁶ is a substituent from Table 4 or Table 5. In some embodiments, -L³-R⁸ is a substituent from Table 6 or Table 7. In some embodiments, R^{A} is a substituent from Table 1, and -L²-R⁶ is a substituent from Table 4 or Table 5. In some embodiments, R^{A} is a substituent from Table 1, and -L³-R⁸ is a substituent from Table 6 or Table 7. In some embodiments, -L²-R⁶ is a substituent from Table 4 or Table 5, and -L³-R⁸ is a substituent from Table 6 or Table 7. In some embodiments, R^{A} is a substituent from Table 1, -L²-R⁶ is a substituent from Table 4 or Table 5, and -L³-R⁸ is a substituent from Table 6 or Table 7.

In some embodiments, the compound of Formula I or Formula I′ is a compound of Formula II:

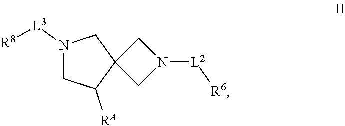

or a pharmaceutically acceptable salt thereof, wherein R^{A}, L², R⁶, L³ and R⁸, and their constituent groups, are each as defined and described herein. In some embodiments, R^{A}, L², R⁶, L³ and R⁸, are as described in Formula I or Formula I′. In some embodiments, R^{A} is a substituent from Table 1. In some embodiments, -L²-R⁶ is a substituent from Table 4 or Table 5. In some embodiments, -L³-R⁸ is a substituent from Table 6 or Table 7. In some embodiments, R^{A} is a substituent from Table 1, and -L²-R⁶ is a substituent from Table 4 or Table 5. In some embodiments, R^{A} is a substituent from Table 1, and -L³-R⁸ is a substituent from Table 6 or Table 7. In some embodiments, -L²-R⁶ is a substituent from Table 4 or Table 5, and -L³-R⁸ is a substituent from Table 6 or Table 7. In some embodiments, R^{A} is a substituent from Table 1, -L²-R⁶ is a substituent from Table 4 or Table 5, and -L³-R⁸ is a substituent from Table 6 or Table 7.

In some embodiments, the compound of Formula I or Formula I′ is a compound of Formula IIIa, IIIb, IIIc, IIId, IIIe, or IIIf:

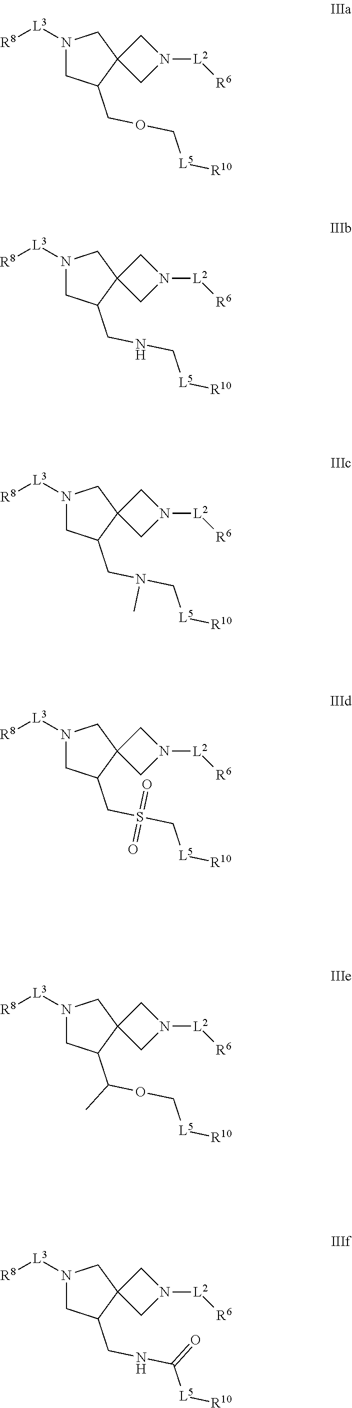

or a pharmaceutically acceptable salt thereof, wherein L⁵, R¹⁰, L², R⁶, L³ and R⁸, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is a covalent bond. In some embodiments, L³ is a methylene. In some embodiments, L³ is a covalent bond. In some embodiments, L² is a —C(O)—. In some embodiments, L³ is a —C(O)—. In some embodiments, both L² and L³ are a covalent bond. In some embodiments, both L² and L³ are —C(O)—. In some embodiments, -L²-R⁶ is a substituent from Table 4 or Table 5. In some embodiments, -L³-R⁸ is a substituent from Table 6 or Table 7.

In some embodiments, the compound of Formula I or Formula I′ is a compound of Formula IVa, IVb, IVc, IVd, IVe, or IVf:

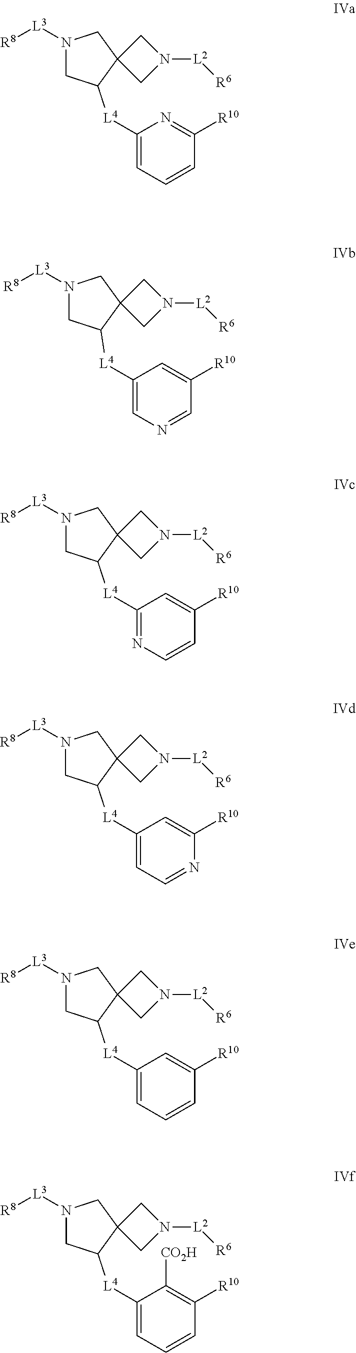

or a pharmaceutically acceptable salt thereof, wherein L⁴, R¹⁰, L², R⁶, L³ and R⁸, and their constituent groups, are each as defined and described herein. In some embodiments, L² is a methylene. In some embodiments, L² is a covalent bond. In some embodiments, L³ is a methylene. In some embodiments, L³ is a covalent bond. In some embodiments, L² is a —C(O)—. In some embodiments, L³ is a —C(O)—. In some embodiments, both L² and L³ are —C(O)—. In some embodiments, -L²-R⁶ is a substituent from Table 4 or Table 5. In some embodiments, -L³-R⁸ is a substituent from Table 6 or Table 7.

In some embodiments, the compound of Formula I or Formula I′ is a compound of Formula Va, Vb, Vc, Vd, Ve, or Vf:

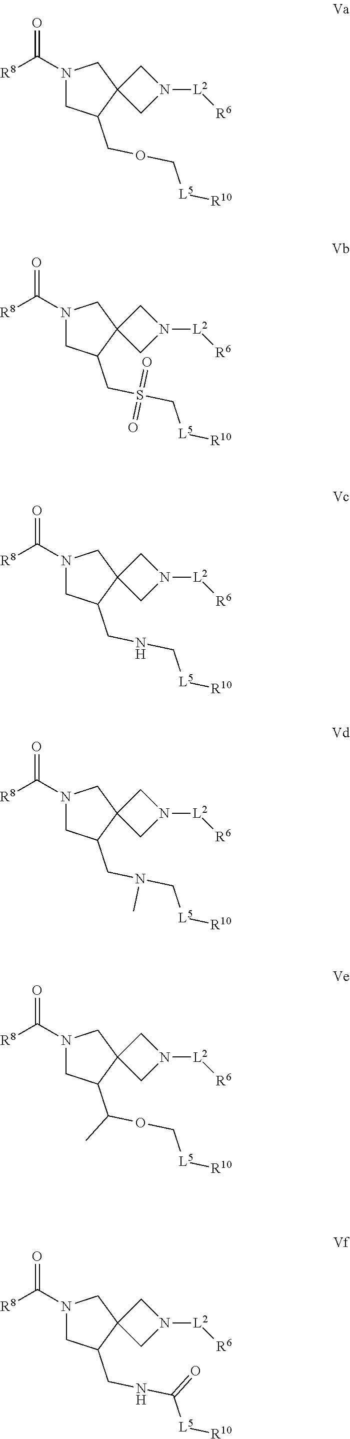

or a pharmaceutically acceptable salt thereof, wherein L⁵, R¹⁰, L², R⁶, and R⁸, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is a —C(O)—. In some embodiments, L² is a covalent bond. In some embodiments, R⁸ is a substituent from Table 7. In some embodiments, R⁶ is a substituent from Table 5. In some embodiments, -L²-R⁶ is a substituent from Table 4 or Table 5.

In some embodiments, the compound of Formula I or Formula I′ is a compound of Formula VIa, VIb, VIc, VId, VIe, or VIf:

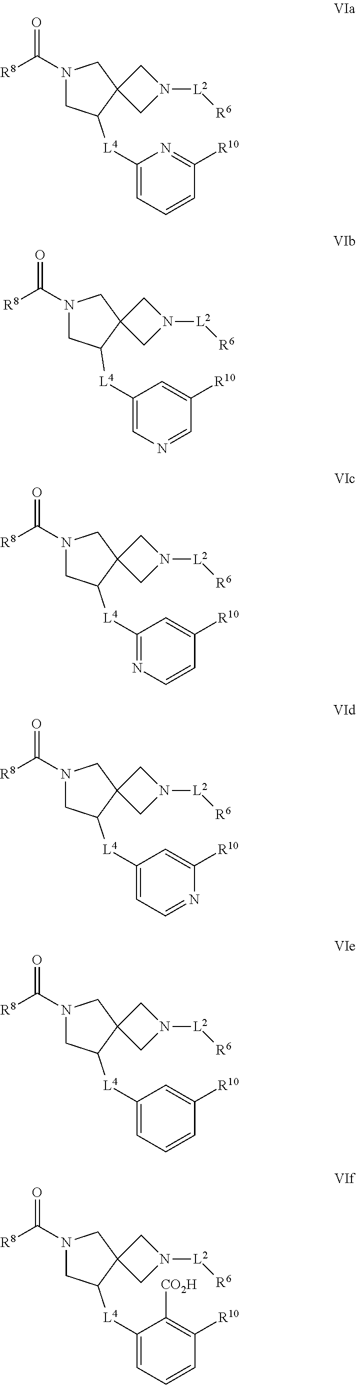

or a pharmaceutically acceptable salt thereof, wherein L⁴, R¹⁰, L², R⁶, and R⁸, and their constituent groups, are each as defined and described herein. In some embodiments, L⁴ is —C(CH₃)HO—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, or —OCH₂—. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is —C(O)—. In some embodiments, L² is a covalent bond. In some embodiments, R⁸ is a substituent from Table 7. In some embodiments, R⁶ is a substituent from Table 5. In some embodiments, -L²-R⁶ is a substituent from Table 4 or Table 5.

In some embodiments, the compound of Formula I or Formula I′ is a compound of Formula VIIa, VIIb, VIIc, VIId, VIIe, or VIIf:

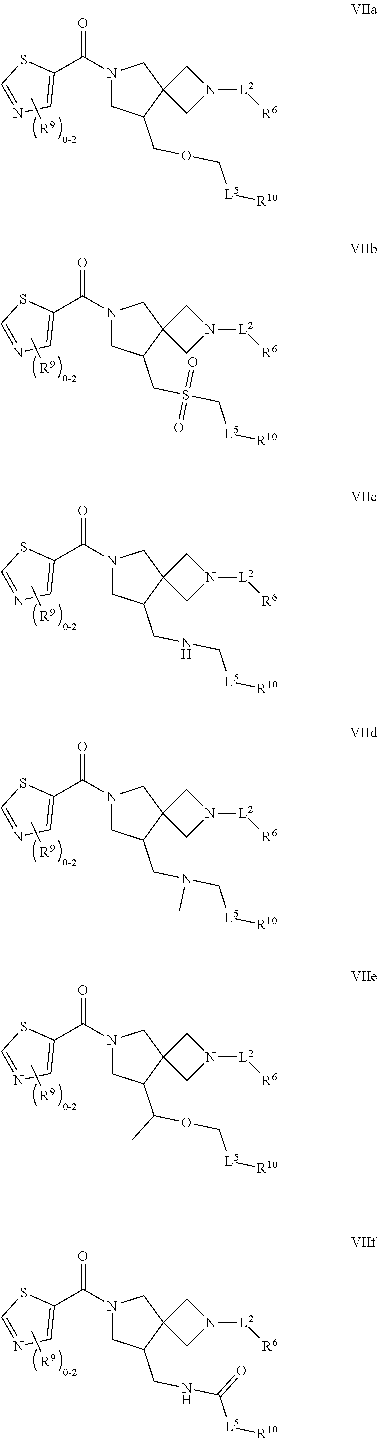

or a pharmaceutically acceptable salt thereof, wherein L⁵, R¹⁰, L², R⁶, and R⁹, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is —C(O)—. In some embodiments, L² is a covalent bond. In some embodiments, R⁶ is a substituent from Table 5. In some embodiments, -L²-R⁶ is a substituent from Table 4 or Table 5.

In some embodiments, the compound of Formula I or Formula I′ is a compound of Formula VIIIa, VIIIb, VIIIc, VIIId, VIIIe, or VIIIf:

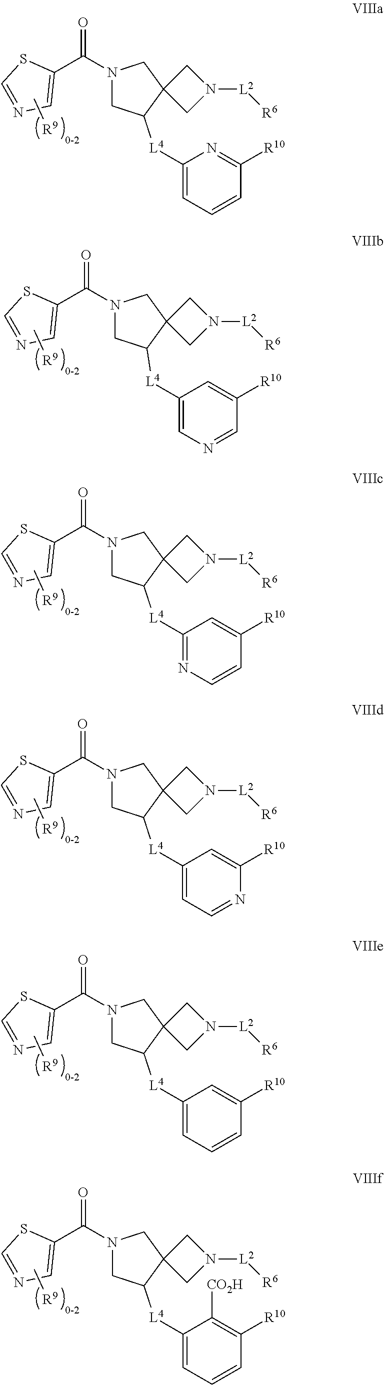

or a pharmaceutically acceptable salt thereof, wherein L⁴, R¹⁰, L², R⁶, and R⁹, and their constituent groups, are each as defined and described herein. In some embodiments, L⁴ is —C(CH₃)HO—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, or —OCH₂—. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is —C(O)—. In some embodiments, L² is a covalent bond. In some embodiments, R⁶ is a substituent from Table 5. In some embodiments, -L²-R⁶ is a substituent from Table 4 or Table 5.

In some embodiments, the compound of Formula I or Formula I′ is a compound of Formula IXa, IXb, IXc, IXd, IXe, or IXf.

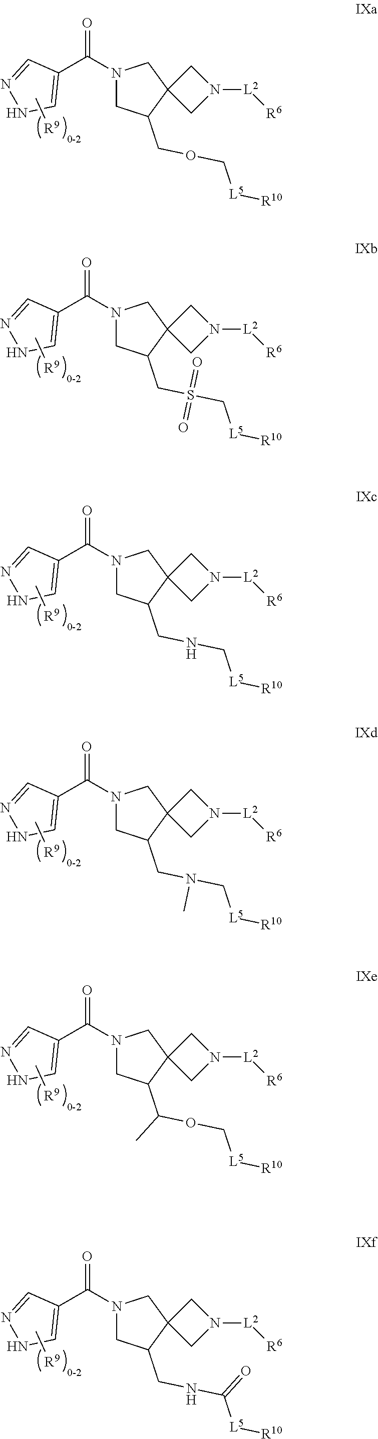

or a pharmaceutically acceptable salt thereof, wherein L⁵, R¹⁰, L², R⁶, and R⁹, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is —C(O)—. In some embodiments, L² is a covalent bond. In some embodiments, R⁶ is a substituent from Table 5. In some embodiments, -L²-R⁶ is a substituent from Table 4 or Table 5.

In some embodiments, the compound of Formula I or Formula I′ is a compound of Formula Xa, Xb, Xc, Xd, Xe, or Xf:

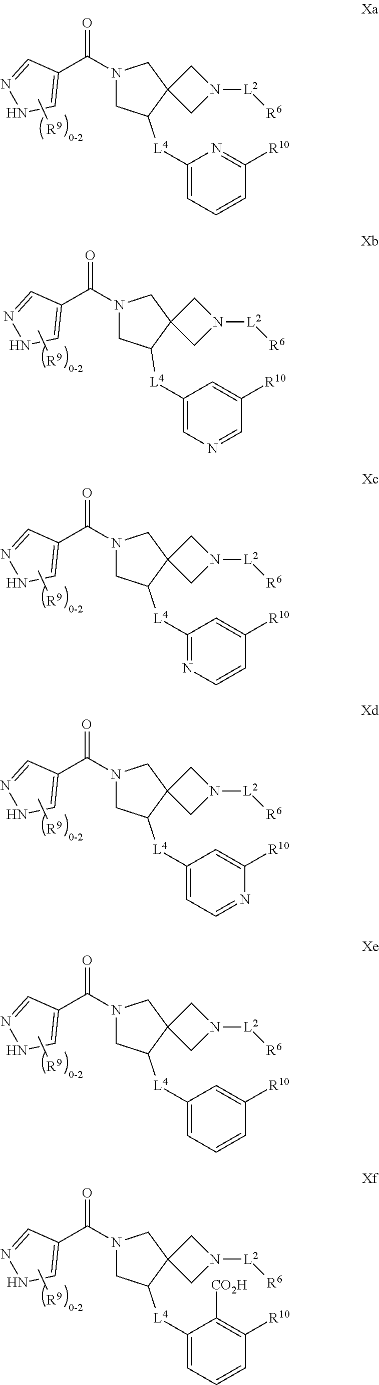

or a pharmaceutically acceptable salt thereof, wherein L⁴, R¹⁰, L², R⁶, and R⁹, and their constituent groups, are each as defined and described herein. In some embodiments, L⁴ is —C(CH₃)HO—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂O—, or —OCH₂—. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L² is —C(O)—. In some embodiments, L² is a covalent bond. In some embodiments, R⁶ is a substituent from Table 5. In some embodiments, -L²-R⁶ is a substituent from Table 4 or Table 5.

In some embodiments, the compound of Formula I or Formula I′ is a compound of Formula XIa, XIb, XIc, XId, XIe, or XIf:

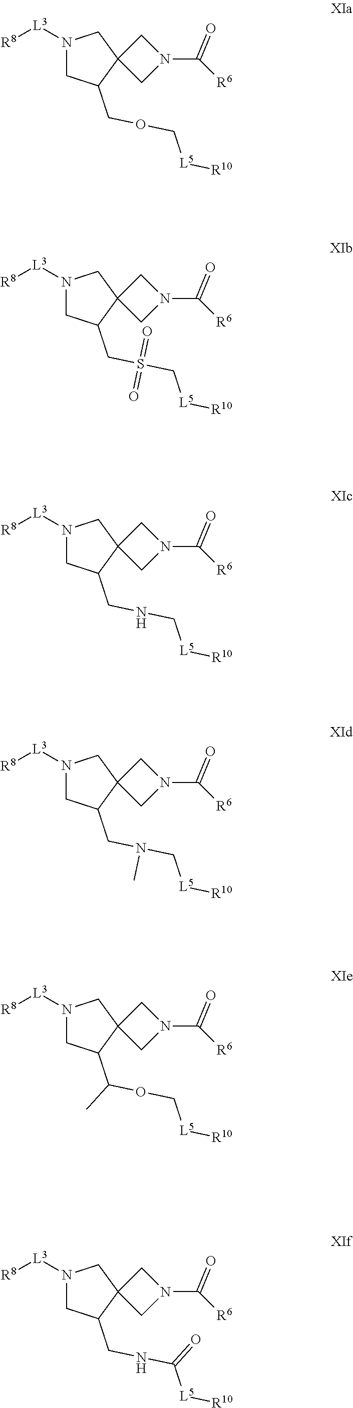

or a pharmaceutically acceptable salt thereof, wherein L, R^{∘}, R⁶, L³ and R⁸, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L³ is a covalent bond. In some embodiments, L³ is a methylene. In some embodiments, L³ is —C(O)—. In some embodiments, R⁶ is a substituent from Table 5. In some embodiments, -L³-R⁸ is a substituent from Table 6 or Table 7.

In some embodiments, the compound of Formula I or Formula I′ is a compound of Formula XIIa, XIIb, XIIc, XIId, XIIe, or XIIf:

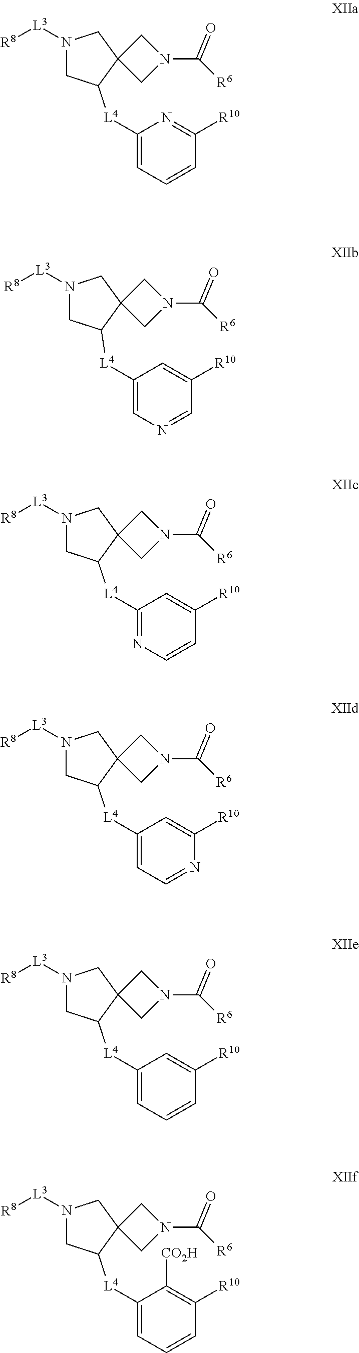

or a pharmaceutically acceptable salt thereof, wherein L⁴, R¹⁰, R⁶, L³ and R⁸, and their constituent groups, are each as defined and described herein. In some embodiments, L³ is a methylene. In some embodiments, L³ is a covalent bond. In some embodiments, L³ is —C(O)—. In some embodiments, R⁶ is a substituent from Table 5. In some embodiments, -L³-R⁸ is a substituent from Table 6 or Table 7.

In some embodiments, the compound of Formula I or Formula I′ is a compound of Formula XIIIa, XIIIb, XIIIc, XIIId, XIIIe, or XIIIf:

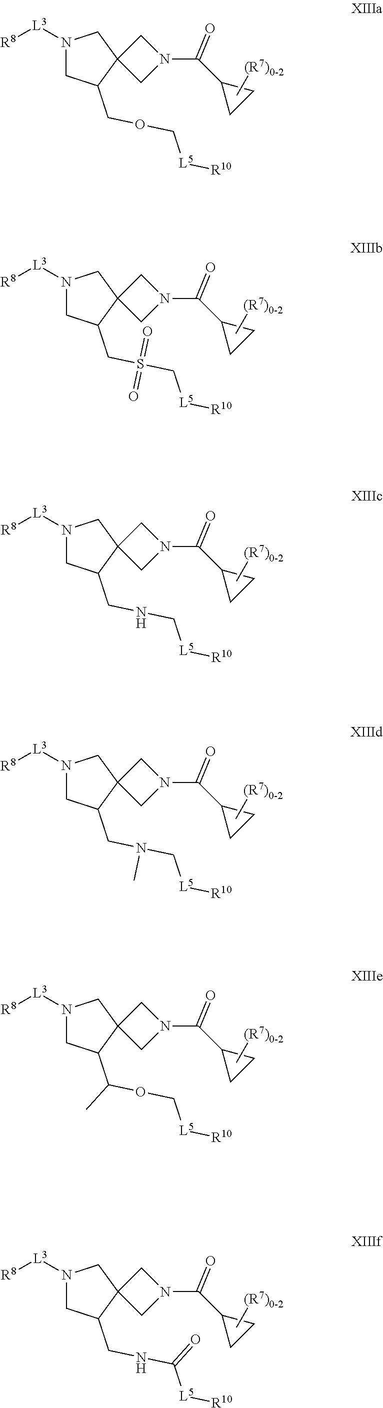

or a pharmaceutically acceptable salt thereof, wherein L⁵, R¹⁰, R⁷, L³ and R⁸, and their constituent groups, are each as defined and described herein. In some embodiments, L⁵ is pyridinylene. In some embodiments, L⁵ is optionally substituted phenylene. In some embodiments, R¹⁰ is a substituent from Table 3. In some embodiments, R¹⁰ is a cyclic group selected from (para-trifluoromethyl)phenyl, tetrahydrofuranyl, cyclohexyl, (para-cyclopropyl)phenyl, (ortho-methoxy)phenyl, pyridinyl, (para-trifluoromethyl)pyridinyl, (para-trifluoromethoxy)cyclohexyl, phenyl, cyclopentyl, or 3,4-dichlorophenyl. In some embodiments, L² is a methylene. In some embodiments, L³ is a covalent bond. In some embodiments, L³ is a methylene. In some embodiments, L³ is —C(O)—. In some embodiments, R⁷ is fluoro, methyl, or trifluoromethyl. In some embodiments, there are two instances of R⁷ that are both fluoro or both methyl. In some embodiments, there are two instances of geminal R⁷ that are both fluoro or both methyl. In some embodiments, -L³-R⁸ is a substituent from Table 6 or Table 7.

In some embodiments, the compound of Formula I or Formula I′ is a compound of Formula XIVa, XIVb, XIVc, XIVd, XIVe, or XIVf:

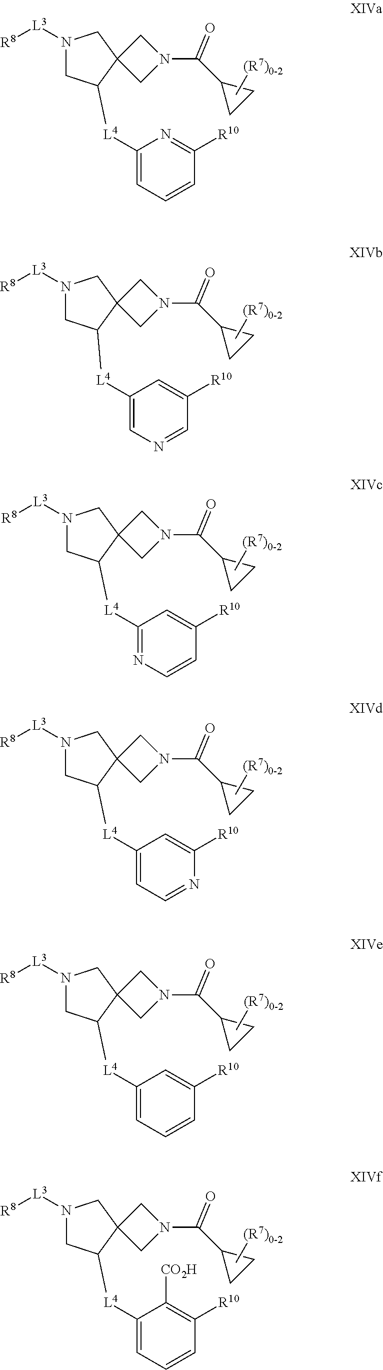

or a pharmaceutically acceptable salt thereof, wherein L⁴, R¹⁰, R⁷, L³ and R⁸, and their constituent groups, are each as defined and described herein. In some embodiments, L³ is a methylene. In some embodiments, L³ is a covalent bond. In some embodiments, L³ is —C(O)—. In some embodiments, R⁷ is fluoro, methyl, or trifluoromethyl. In some embodiments, there are two instances of R⁷ that are both fluoro or both methyl. In some embodiments, there are two instances of geminal R⁷ that are both fluoro or both methyl. In some embodiments, -L³-R⁸ is a substituent from Table 6 or Table 7.

In some embodiments, the compound of Formula I or Formula I′ is a compound of Formula XVa, XVb, XVc, XVd, or XVe:

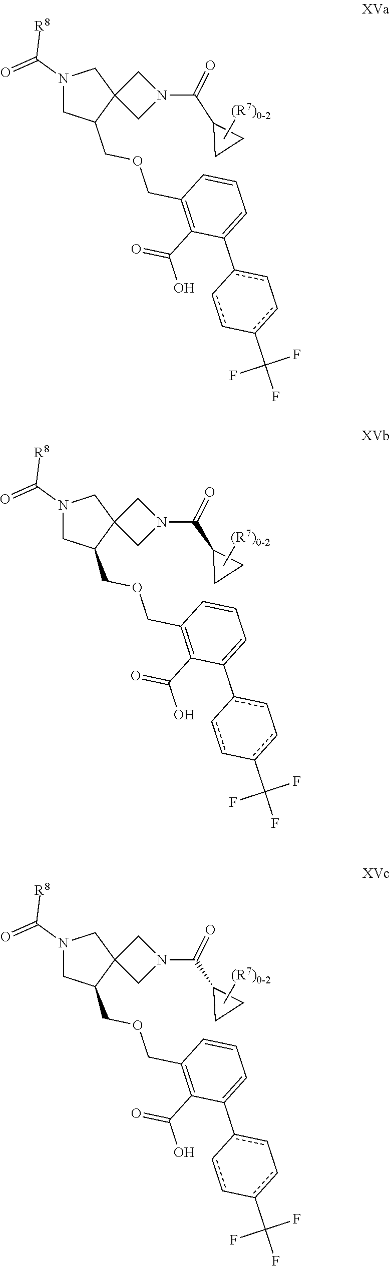

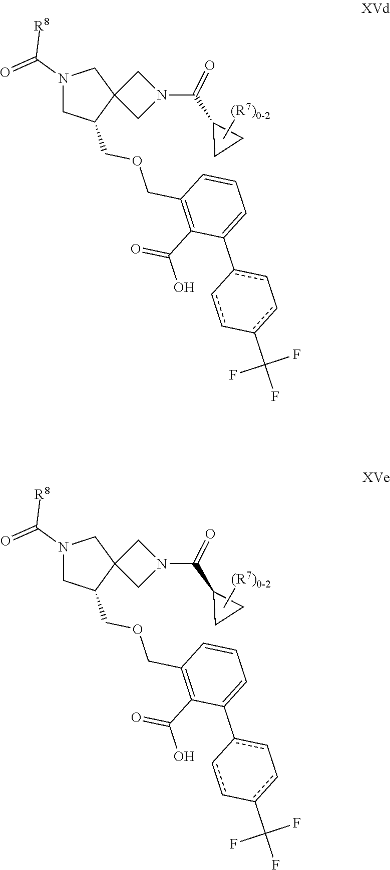

or a pharmaceutically acceptable salt thereof, wherein R⁸, and its constituent groups, are each as defined and described herein, and wherein each instance of R⁷ is independently —CF₃ or —CH₃, and wherein all instances of [symbol] are a single bond; all instances of [symbol] are a double bond; or any two instances of [symbol] are a single bond and the remaining one instance of [symbol] is a double bond.

In some embodiments, the compound of Formula I or Formula I′ is a compound of Formula XVIa, XVIb, XVIc, XVId, or XVIe:

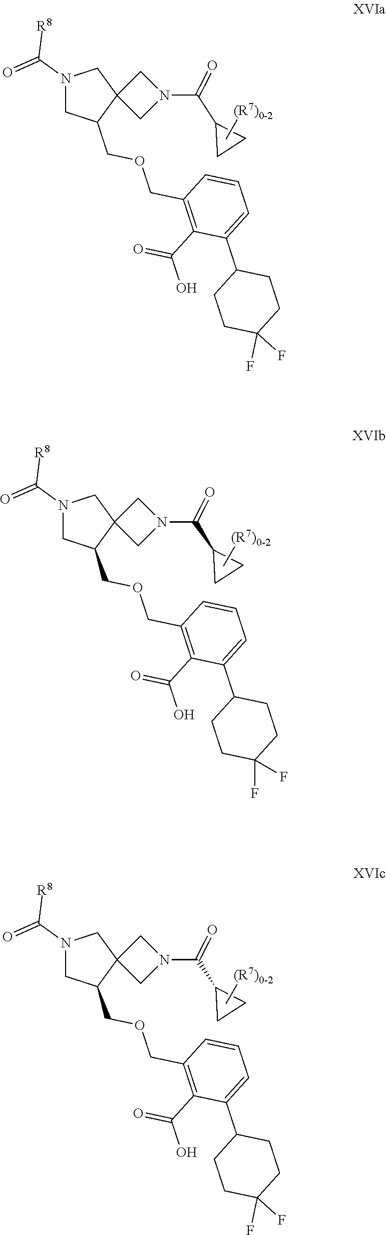

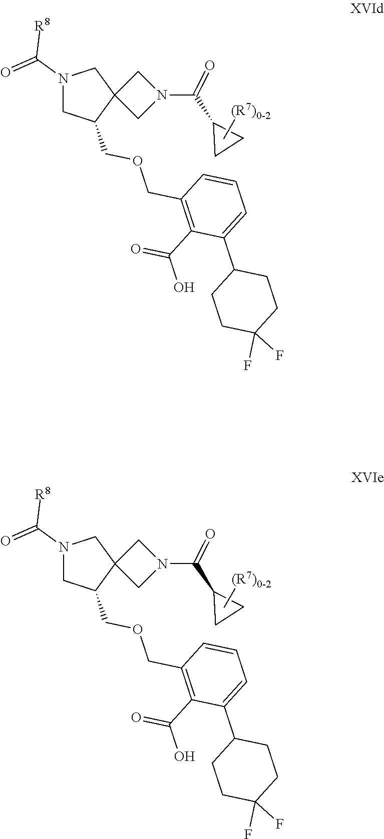

or a pharmaceutically acceptable salt thereof, wherein R⁸, and its constituent groups, are each as defined and described herein, and wherein each instance of R⁷ is independently —CF₃ or —CH₃.

In some embodiments, the compound of Formula I or Formula I′ is a compound of Formula XVIIa, XVIIb, XVIIc, XVIId, or XVIIe:

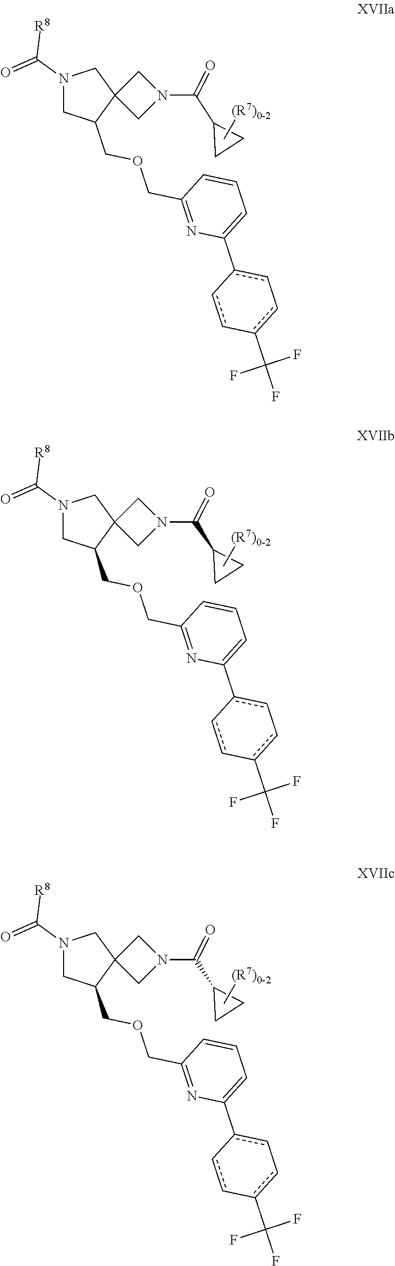

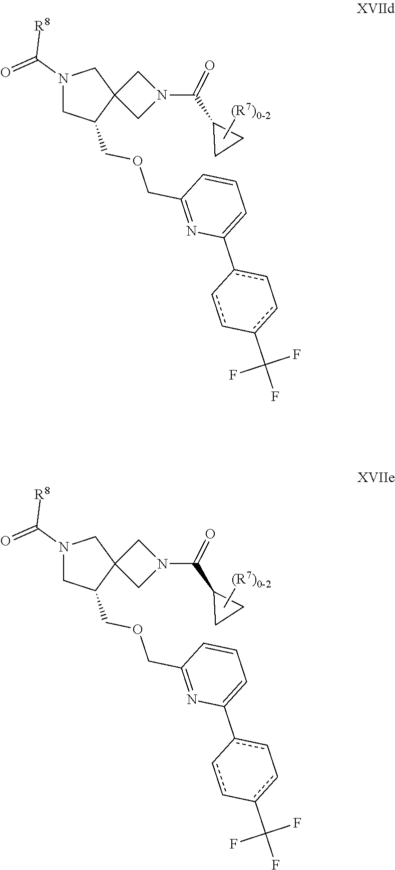

or a pharmaceutically acceptable salt thereof, wherein R⁸, and its constituent groups, are each as defined and described herein, and wherein each instance of R⁷ is independently —CF₃ or —CH₃, and wherein all instances of [symbol] are a single bond; all instances of [symbol] are a double bond; or any two instances of [symbol] are a single bond and the remaining one instance of [symbol] is a double bond.

Exemplary compounds of the present disclosure are set forth in Table 8A and Table 8B, below.

TABLE 8A
Exemplary Compounds

| Compound | |
|---|---|
| No. | Structure |
| I-1 | 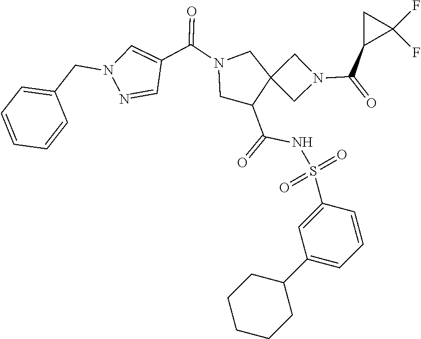 [SMILES: O=C(NS(=O)(=O)c1cccc(C2CCCCC2)c1)C1CN(C(=O)c2cnn(Cc3ccccc3)c2)CC12CN(C(=O)[C@H]1CC1(F)F)C2] |
| I-2 | 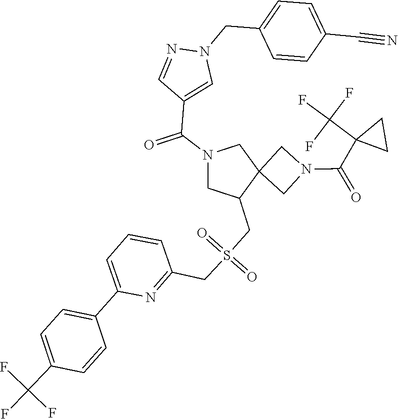 [SMILES: N#Cc1ccc(Cn2cc(C(=O)N3CC(CS(=O)(=O)Cc4cccc(-c5ccc(C(F)(F)F)cc5)n4)C4(C3)CN(C(=O)C3(C(F)(F)F)CC3)C4)cn2)cc1] |
| I-3 | 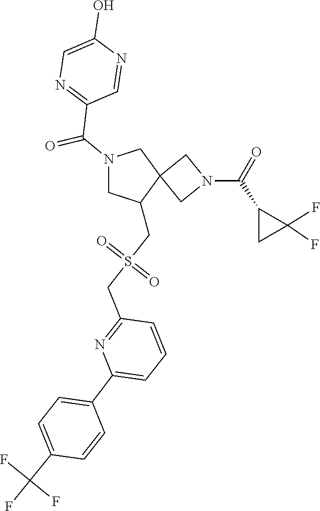 [SMILES: O=C(c1cnc(O)cn1)N1CC(CS(=O)(=O)Cc2cccc(-c3ccc(C(F)(F)F)cc3)n2)C2(C1)CN(C(=O)[C@H]1CC1(F)F)C2] |
| I-4 | 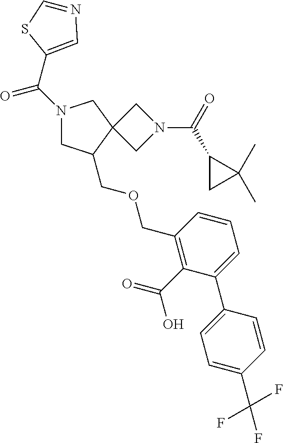 [SMILES: CC1(C)C[C@@H]1C(=O)N1CC2(CN(C(=O)c3cncs3)CC2COCc2cccc(-c3ccc(C(F)(F)F)cc3)c2C(=O)O)C1] |
| I-5 | 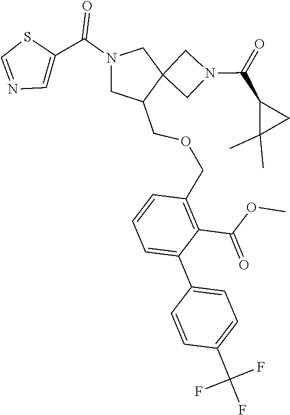 [SMILES: COC(=O)c1c(COCC2CN(C(=O)c3cncs3)CC23CN(C(=O)[C@H]2CC2(C)C)C3)cccc1-c1ccc(C(F)(F)F)cc1] |
| I-6 | 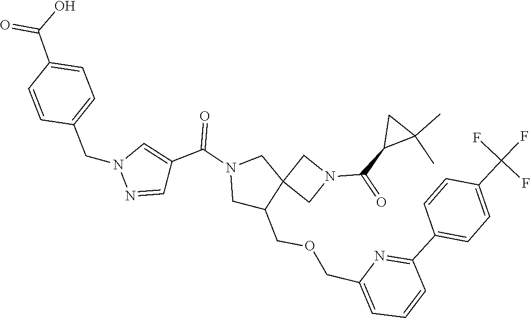 [SMILES: CC1(C)C[C@@H]1C(=O)N1CC2(CN(C(=O)c3cnn(Cc4ccc(C(=O)O)cc4)c3)CC2COCc2cccc(-c3ccc(C(F)(F)F)cc3)n2)C1] |
| I-7 | 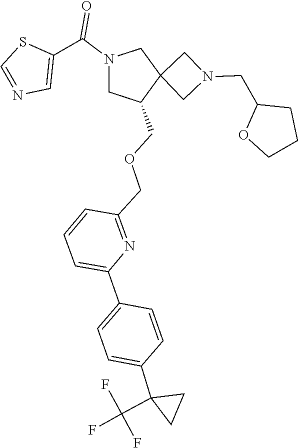 [SMILES: O=C(c1cncs1)N1C[C@@H](COCc2cccc(-c3ccc(C4(C(F)(F)F)CC4)cc3)n2)C2(CN(CC3CCCO3)C2)C1] |
| I-8 | 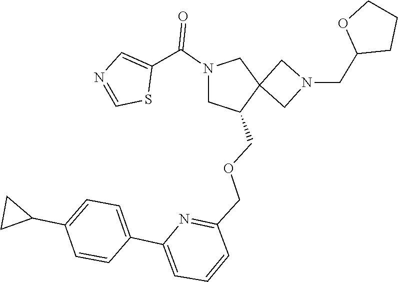 [SMILES: O=C(c1cncs1)N1C[C@@H](COCc2cccc(-c3ccc(C4CC4)cc3)n2)C2(CN(CC3CCCO3)C2)C1] |
| I-9 | 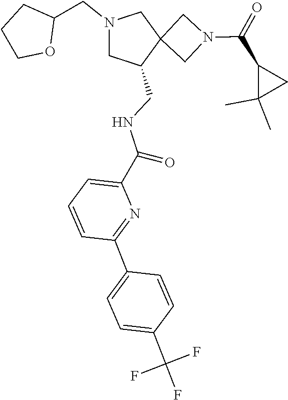 [SMILES: CC1(C)C[C@@H]1C(=O)N1CC2(CN(CC3CCCO3)C[C@H]2CNC(=O)c2cccc(-c3ccc(C(F)(F)F)cc3)n2)C1] |
| I-10 | 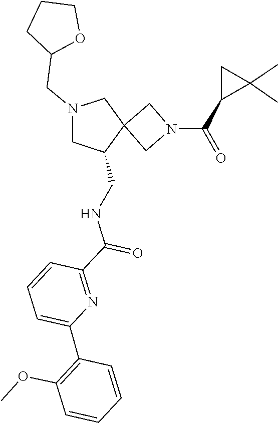 |
| I-11 | 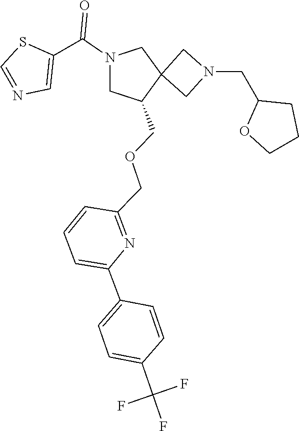 [SMILES: O=C(c1cncs1)N1C[C@@H](COCc2cccc(-c3ccc(C(F)(F)F)cc3)n2)C2(CN(CC3CCCO3)C2)C1] |
| I-12 | 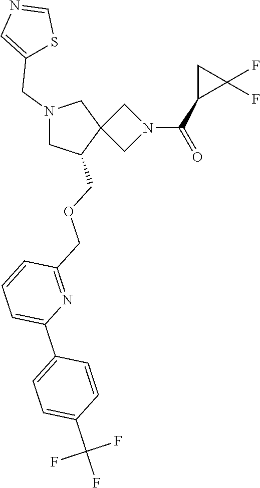 [SMILES: O=C([C@H]1CC1(F)F)N1CC2(CN(Cc3cncs3)C[C@H]2COCc2cccc(-c3ccc(C(F)(F)F)cc3)n2)C1] |
| I-13 | 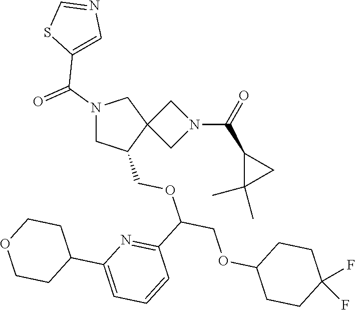 [SMILES: CC1(C)C[C@@H]1C(=O)N1CC2(CN(C(=O)c3cncs3)C[C@H]2COC(COC2CCC(F)(F)CC2)c2cccc(C3CCOCC3)n2)C1] |
| I-14 | 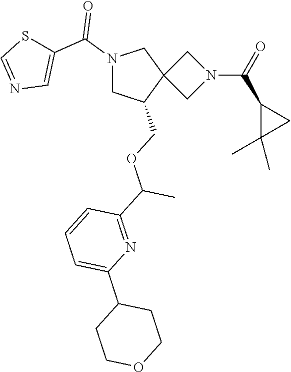 [SMILES: CC(OC[C@@H]1CN(C(=O)c2cncs2)CC12CN(C(=O)[C@H]1CC1(C)C)C2)c1cccc(C2CCOCC2)n1] |
| I-15 | 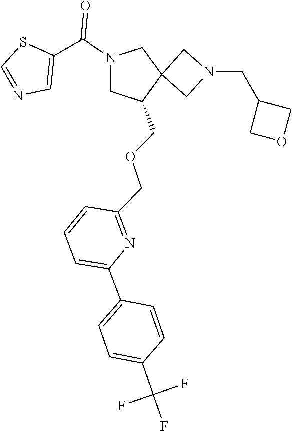 [SMILES: O=C(c1cncs1)N1C[C@@H](COCc2cccc(-c3ccc(C(F)(F)F)cc3)n2)C2(CN(CC3COC3)C2)C1] |
| I-16 | 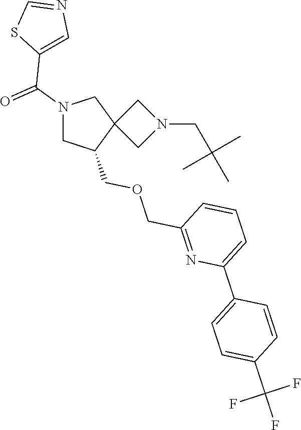 [SMILES: CC(C)(C)CN1CC2(C1)CN(C(=O)c1cncs1)C[C@H]2COCc1cccc(-c2ccc(C(F)(F)F)cc2)n1] |
| I-17 | 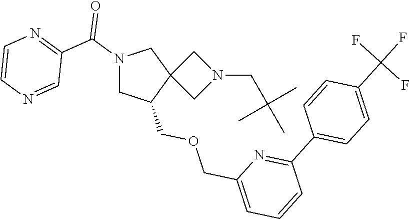 [SMILES: CC(C)(C)CN1CC2(C1)CN(C(=O)c1cnccn1)C[C@H]2COCc1cccc(-c2ccc(C(F)(F)F)cc2)n1] |
| I-18 | 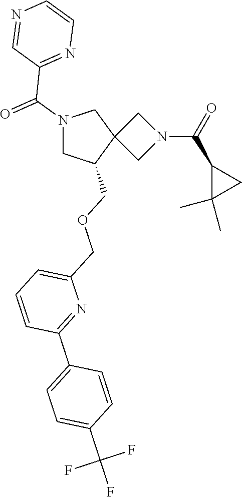 [SMILES: CC1(C)C[C@@H]1C(=O)N1CC2(CN(C(=O)c3cnccn3)C[C@H]2COCc2cccc(-c3ccc(C(F)(F)F)cc3)n2)C1] |
| I-19 | 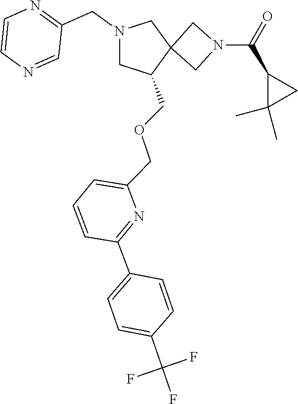 [SMILES: CC1(C)C[C@@H]1C(=O)N1CC2(CN(Cc3cnccn3)C[C@H]2COCc2cccc(-c3ccc(C(F)(F)F)cc3)n2)C1] |
| I-20 | 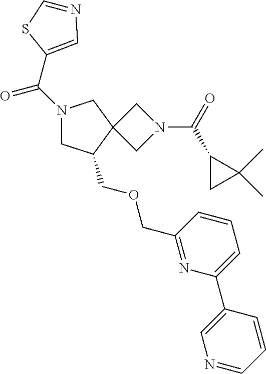 [SMILES: CC1(C)C[C@@H]1C(=O)N1CC2(CN(C(=O)c3cncs3)C[C@H]2COCc2cccc(-c3cccnc3)n2)C1] |
| I-21 | 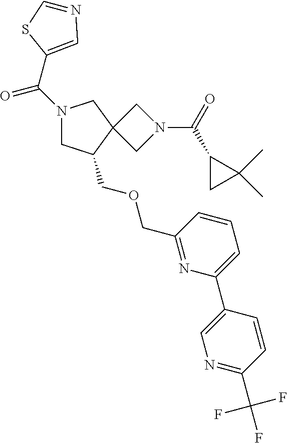 [SMILES: CC1(C)C[C@@H]1C(=O)N1CC2(CN(C(=O)c3cncs3)C[C@H]2COCc2cccc(-c3ccc(C(F)(F)F)nc3)n2)C1] |
| I-22 | 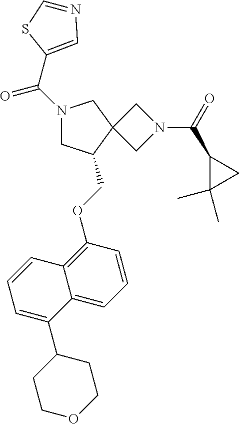 [SMILES: CC1(C)C[C@@H]1C(=O)N1CC2(CN(C(=O)c3cncs3)C[C@H]2COc2cccc3c(C4CCOCC4)cccc23)C1] |
| I-23 | 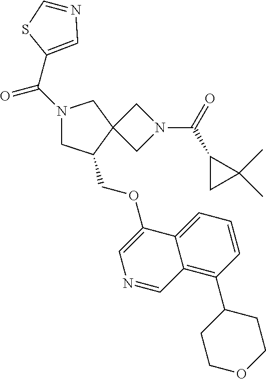 [SMILES: CC1(C)C[C@@H]1C(=O)N1CC2(CN(C(=O)c3cncs3)C[C@H]2COc2cncc3c(C4CCOCC4)cccc23)C1] |
| I-24 | 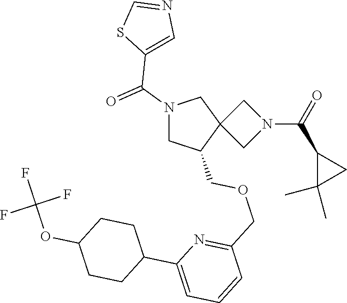 [SMILES: CC1(C)C[C@@H]1C(=O)N1CC2(CN(C(=O)c3cncs3)C[C@H]2COCc2cccc(C3CCC(OC(F)(F)F)CC3)n2)C1] |
| I-25 | 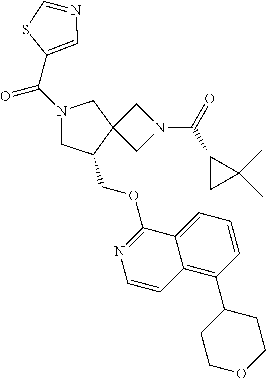 [SMILES: CC1(C)C[C@@H]1C(=O)N1CC2(CN(C(=O)c3cncs3)C[C@H]2COc2nccc3c(C4CCOCC4)cccc23)C1] |
| I-26 | 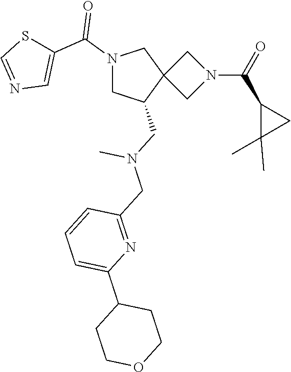 [SMILES: CN(Cc1cccc(C2CCOCC2)n1)C[C@@H]1CN(C(=O)c2cncs2)CC12CN(C(=O)[C@H]1CC1(C)C)C2] |
| I-27 | 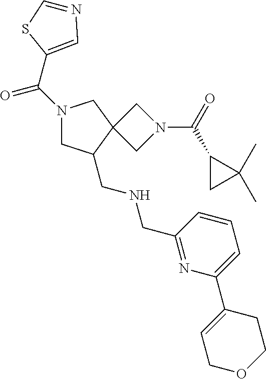 [SMILES: CC1(C)C[C@@H]1C(=O)N1CC2(CN(C(=O)c3cncs3)CC2CNCc2cccc(C3=CCOCC3)n2)C1] |
| I-28 | 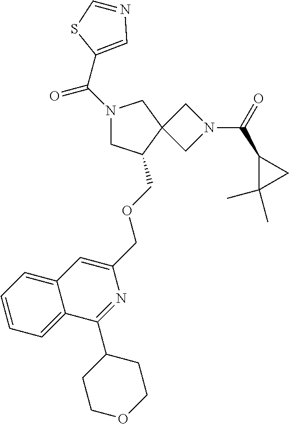 [SMILES: CC1(C)C[C@@H]1C(=O)N1CC2(CN(C(=O)c3cncs3)C[C@H]2COCc2cc3ccccc3c(C3CCOCC3)n2)C1] |
| I-29 | 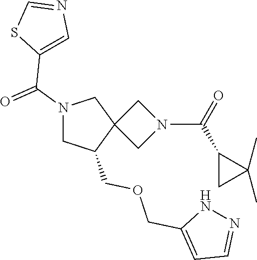 [SMILES: CC1(C)C[C@@H]1C(=O)N1CC2(CN(C(=O)c3cncs3)C[C@H]2COCc2ccn[nH]2)C1] |
| I-30 | 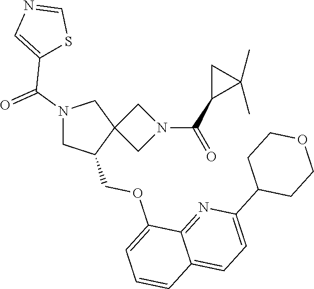 [SMILES: CC1(C)C[C@@H]1C(=O)N1CC2(CN(C(=O)c3cncs3)C[C@H]2COc2cccc3ccc(C4CCOCC4)nc23)C1] |
| I-31 | 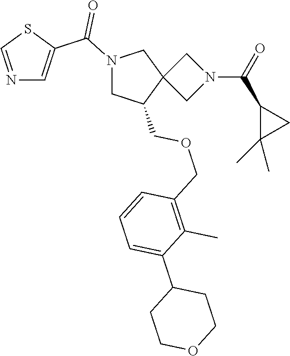 [SMILES: Cc1c(COC[C@@H]2CN(C(=O)c3cncs3)CC23CN(C(=O)[C@H]2CC2(C)C)C3)cccc1C1CCOCC1] |
| I-32 | 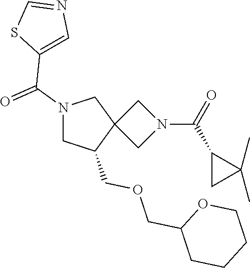 [SMILES: CC1(C)C[C@@H]1C(=O)N1CC2(CN(C(=O)c3cncs3)C[C@H]2COCC2CCCCO2)C1] |
| I-33 | 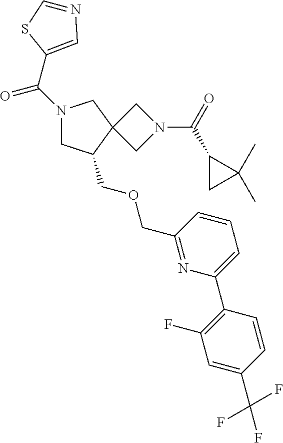 [SMILES: CC1(C)C[C@@H]1C(=O)N1CC2(CN(C(=O)c3cncs3)C[C@H]2COCc2cccc(-c3ccc(C(F)(F)F)cc3F)n2)C1] |
| I-34 | 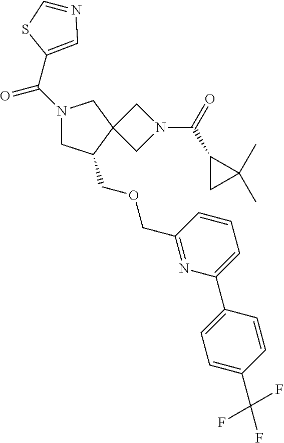 [SMILES: CC1(C)C[C@@H]1C(=O)N1CC2(CN(C(=O)c3cncs3)C[C@H]2COCc2cccc(-c3ccc(C(F)(F)F)cc3)n2)C1] |
| I-35 | 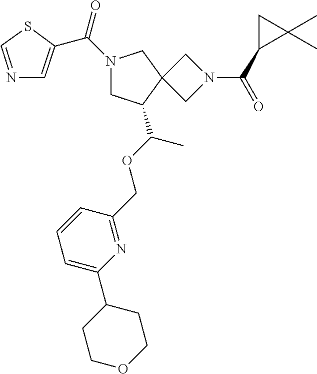 [SMILES: CC(OCc1cccc(C2CCOCC2)n1)[C@@H]1CN(C(=O)c2cncs2)CC12CN(C(=O)[C@H]1CC1(C)C)C2] |
| I-36 | 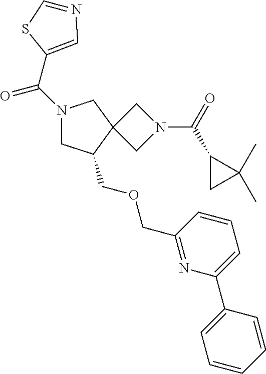 [SMILES: CC1(C)C[C@@H]1C(=O)N1CC2(CN(C(=O)c3cncs3)C[C@H]2COCc2cccc(-c3ccccc3)n2)C1] |
| I-37 | 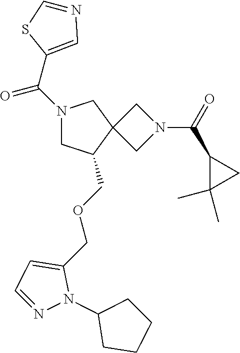 [SMILES: CC1(C)C[C@@H]1C(=O)N1CC2(CN(C(=O)c3cncs3)C[C@H]2COCc2ccnn2C2CCCC2)C1] |
| I-38 | 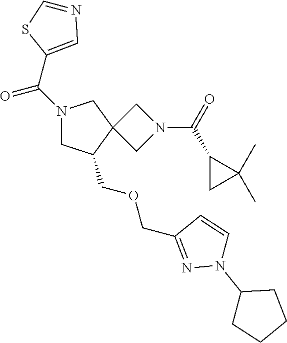 [SMILES: CC1(C)C[C@@H]1C(=O)N1CC2(CN(C(=O)c3cncs3)C[C@H]2COCc2ccn(C3CCCC3)n2)C1] |
| I-39 | 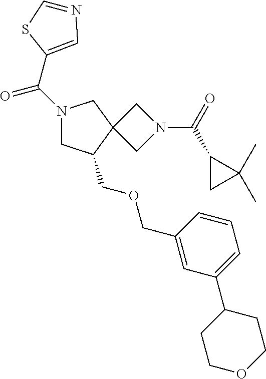 [SMILES: CC1(C)C[C@@H]1C(=O)N1CC2(CN(C(=O)c3cncs3)C[C@H]2COCc2cccc(C3CCOCC3)c2)C1] |
| I-40 | 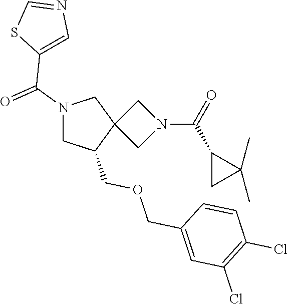 [SMILES: CC1(C)C[C@@H]1C(=O)N1CC2(CN(C(=O)c3cncs3)C[C@H]2COCc2ccc(Cl)c(Cl)c2)C1] |
| I-41 | 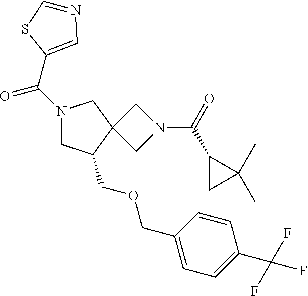 [SMILES: CC1(C)C[C@@H]1C(=O)N1CC2(CN(C(=O)c3cncs3)C[C@H]2COCc2ccc(C(F)(F)F)cc2)C1] |
| I-42 | 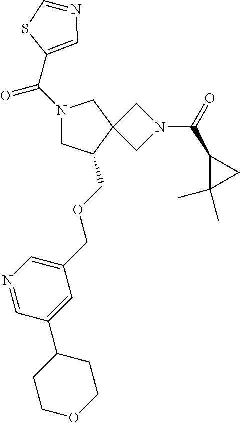 [SMILES: CC1(C)C[C@@H]1C(=O)N1CC2(CN(C(=O)c3cncs3)C[C@H]2COCc2cncc(C3CCOCC3)c2)C1] |
| I-43 | 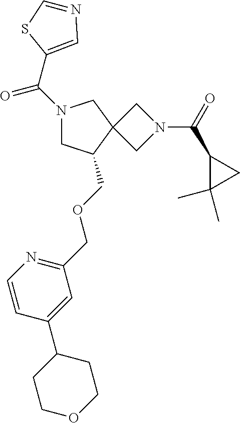 [SMILES: CC1(C)C[C@@H]1C(=O)N1CC2(CN(C(=O)c3cncs3)C[C@H]2COCc2cc(C3CCOCC3)ccn2)C1] |
| I-44 | 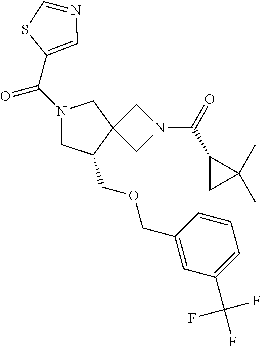 [SMILES: CC1(C)C[C@@H]1C(=O)N1CC2(CN(C(=O)c3cncs3)C[C@H]2COCc2cccc(C(F)(F)F)c2)C1] |
| I-45 | 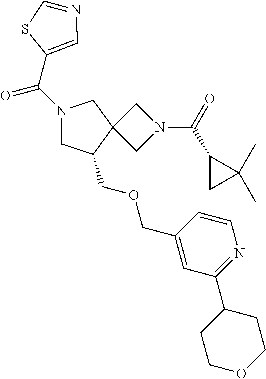 [SMILES: CC1(C)C[C@@H]1C(=O)N1CC2(CN(C(=O)c3cncs3)C[C@H]2COCc2ccnc(C3CCOCC3)c2)C1] |
| I-46 | 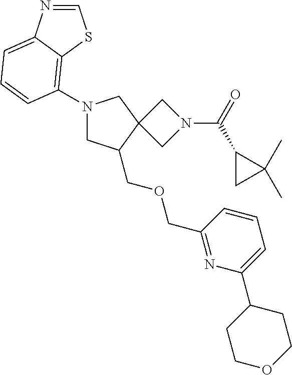 [SMILES: CC1(C)C[C@@H]1C(=O)N1CC2(C1)CN(c1cccc3ncsc13)CC2COCc1cccc(C2CCOCC2)n1] |
| I-47 | 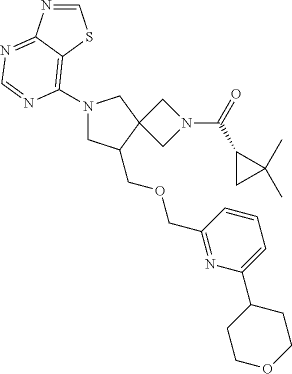 [SMILES: CC1(C)C[C@@H]1C(=O)N1CC2(C1)CN(c1ncnc3ncsc13)CC2COCc1cccc(C2CCOCC2)n1] |
| I-48 | 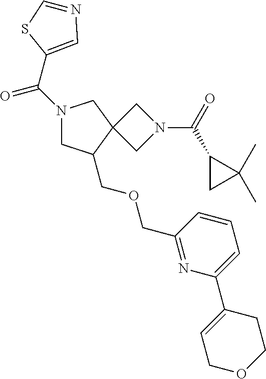 [SMILES: CC1(C)C[C@@H]1C(=O)N1CC2(CN(C(=O)c3cncs3)CC2COCc2cccc(C3=CCOCC3)n2)C1] |
| I-49 | 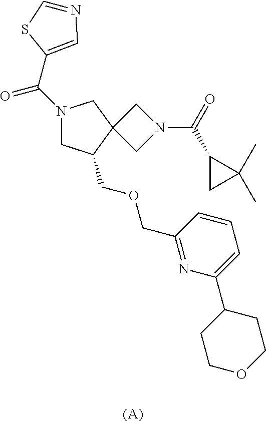 [SMILES: CC1(C)C[C@@H]1C(=O)N1CC2(CN(C(=O)c3cncs3)C[C@H]2COCc2cccc(C3CCOCC3)n2)C1 ; "(A)"] |
| | 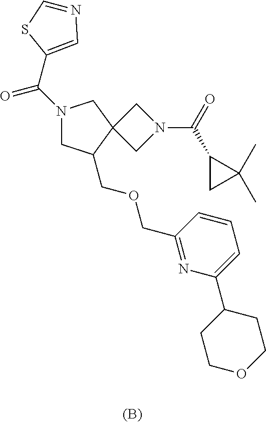 [SMILES: CC1(C)C[C@@H]1C(=O)N1CC2(CN(C(=O)c3cncs3)CC2COCc2cccc(C3CCOCC3)n2)C1 ; "(B)"] |
| I-50 | 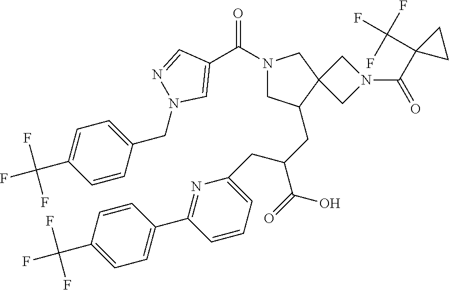 [SMILES: O=C(O)C(Cc1cccc(-c2ccc(C(F)(F)F)cc2)n1)CC1CN(C(=O)c2cnn(Cc3ccc(C(F)(F)F)cc3)c2)CC12CN(C(=O)C1(C(F)(F)F)CC1)C2] |
| I-51 | 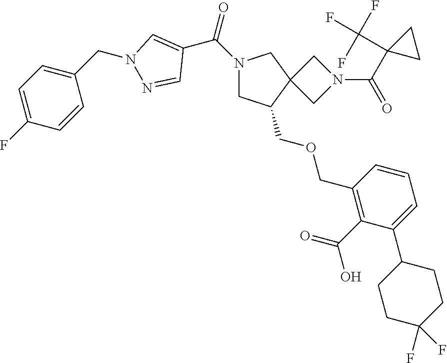 [SMILES: O=C(O)c1c(COC[C@@H]2CN(C(=O)c3cnn(Cc4ccc(F)cc4)c3)CC23CN(C(=O)C2(C(F)(F)F)CC2)C3)cccc1C1CCC(F)(F)CC1] |
| I-52 | 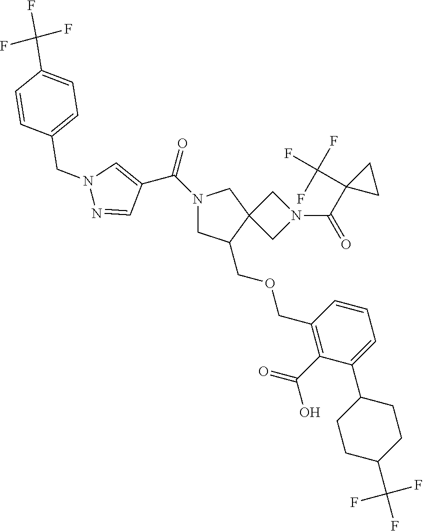 [SMILES: O=C(O)c1c(COCC2CN(C(=O)c3cnn(Cc4ccc(C(F)(F)F)cc4)c3)CC23CN(C(=O)C2(C(F)(F)F)CC2)C3)cccc1C1CCC(C(F)(F)F)CC1] |
| I-53 | 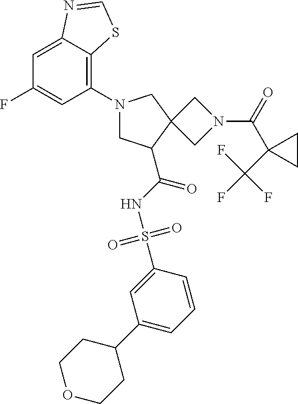 [SMILES: O=C(NS(=O)(=O)c1cccc(C2CCOCC2)c1)C1CN(c2cc(F)cc3ncsc23)CC12CN(C(=O)C1(C(F)(F)F)CC1)C2] |
| I-54 | 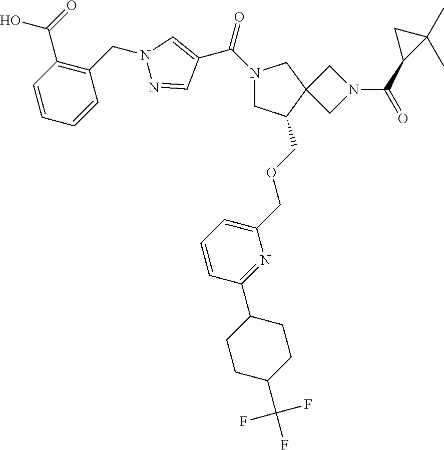 [SMILES: CC1(C)C[C@@H]1C(=O)N1CC2(CN(C(=O)c3cnn(Cc4ccccc4C(=O)O)c3)C[C@H]2COCc2cccc(C3CCC(C(F)(F)F)CC3)n2)C1] |
| I-55 | 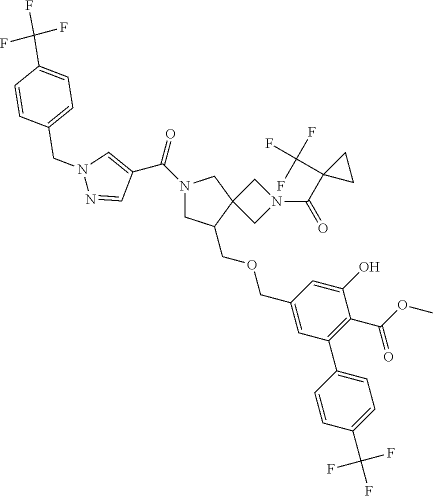 [SMILES: COC(=O)c1c(O)cc(COCC2CN(C(=O)c3cnn(Cc4ccc(C(F)(F)F)cc4)c3)CC23CN(C(=O)C2(C(F)(F)F)CC2)C3)cc1-c1ccc(C(F)(F)F)cc1] |
| I-56 | 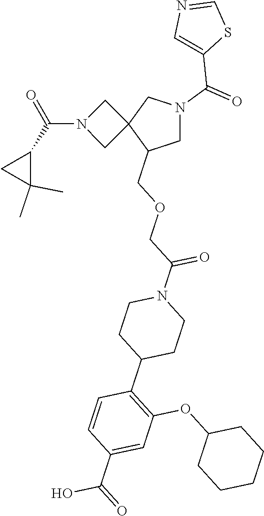 [SMILES: CC1(C)C[C@@H]1C(=O)N1CC2(CN(C(=O)c3cncs3)CC2COCC(=O)N2CCC(c3ccc(C(=O)O)cc3OC3CCCCC3)CC2)C1] |
| I-57 | 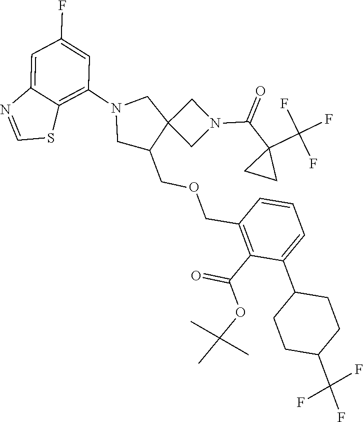 [SMILES: CC(C)(C)OC(=O)c1c(COCC2CN(c3cc(F)cc4ncsc34)CC23CN(C(=O)C2(C(F)(F)F)CC2)C3)cccc1C1CCC(C(F)(F)F)CC1] |
| I-58 | 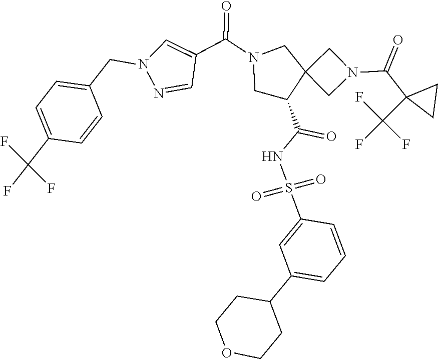 [SMILES: O=C(NS(=O)(=O)c1cccc(C2CCOCC2)c1)[C@@H]1CN(C(=O)c2cnn(Cc3ccc(C(F)(F)F)cc3)c2)CC12CN(C(=O)C1(C(F)(F)F)CC1)C2] |
| I-59 | 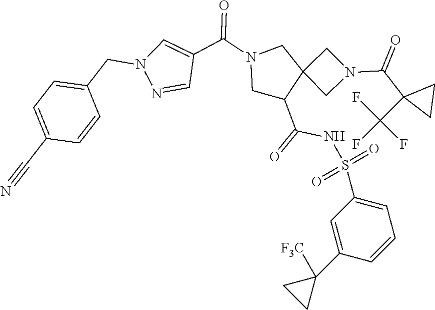 [SMILES: N#Cc1ccc(Cn2cc(C(=O)N3CC(C(=O)NS(=O)(=O)c4cccc(C5(C(F)(F)F)CC5)c4)C4(C3)CN(C(=O)C3(C(F)(F)F)CC3)C4)cn2)cc1] |
| I-60 | 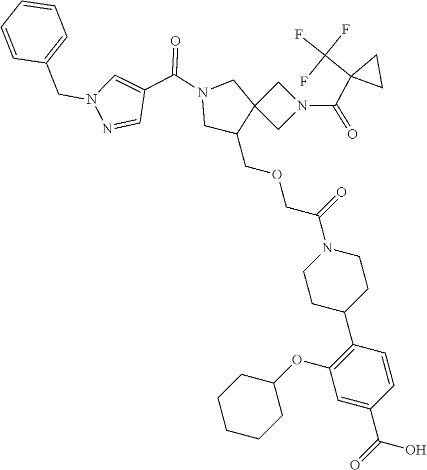 [SMILES: O=C(O)c1ccc(C2CCN(C(=O)COCC3CN(C(=O)c4cnn(Cc5ccccc5)c4)CC34CN(C(=O)C3(C(F)(F)F)CC3)C4)CC2)c(OC2CCCCC2)c1] |
| I-61 | 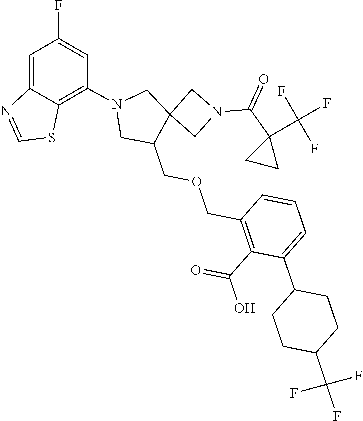 [SMILES: O=C(O)c1c(COCC2CN(c3cc(F)cc4ncsc34)CC23CN(C(=O)C2(C(F)(F)F)CC2)C3)cccc1C1CCC(C(F)(F)F)CC1] |
| I-62 | 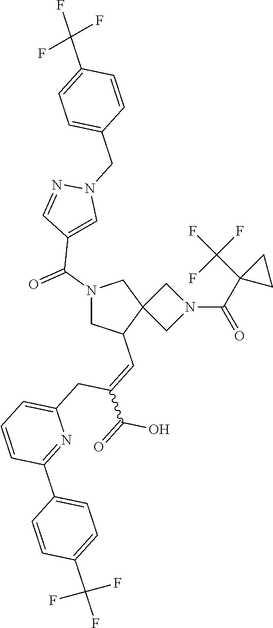 [SMILES: O=C(O)C(=CC1CN(C(=O)c2cnn(Cc3ccc(C(F)(F)F)cc3)c2)CC12CN(C(=O)C1(C(F)(F)F)CC1)C2)Cc1cccc(-c2ccc(C(F)(F)F)cc2)n1] |
| I-63 | 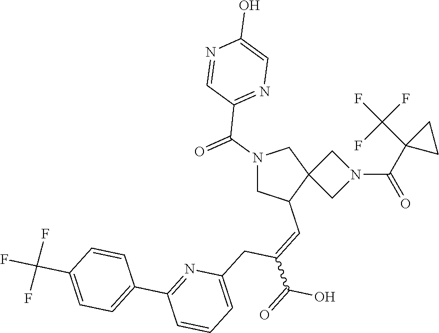 [SMILES: O=C(O)C(=CC1CN(C(=O)c2cnc(O)cn2)CC12CN(C(=O)C1(C(F)(F)F)CC1)C2)Cc1cccc(-c2ccc(C(F)(F)F)cc2)n1] |
| I-64 | 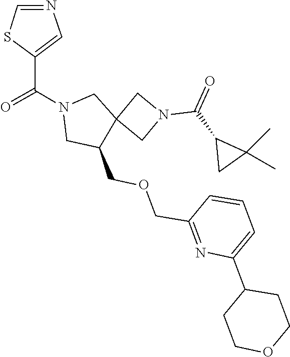 [SMILES: CC1(C)C[C@@H]1C(=O)N1CC2(CN(C(=O)c3cncs3)C[C@@H]2COCc2cccc(C3CCOCC3)n2)C1] |
| I-65 | 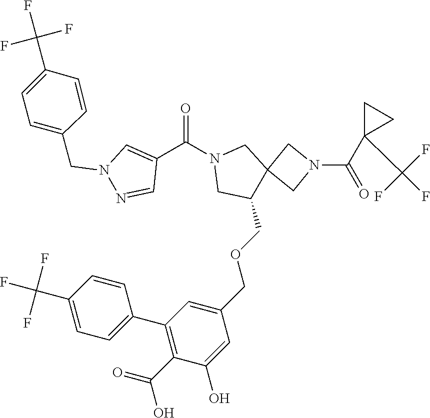 [SMILES: O=C(O)c1c(O)cc(COC[C@@H]2CN(C(=O)c3cnn(Cc4ccc(C(F)(F)F)cc4)c3)CC23CN(C(=O)C2(C(F)(F)F)CC2)C3)cc1-c1ccc(C(F)(F)F)cc1] |
| I-66 | 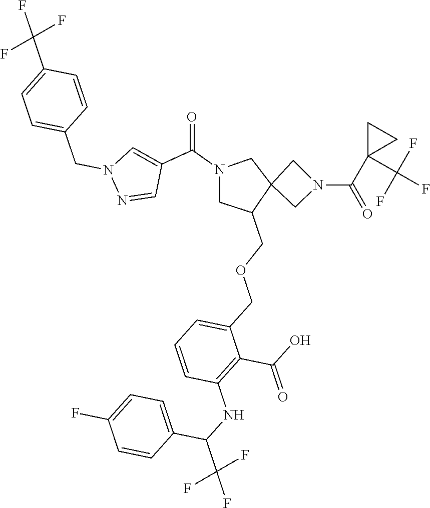 [SMILES: O=C(O)c1c(COCC2CN(C(=O)c3cnn(Cc4ccc(C(F)(F)F)cc4)c3)CC23CN(C(=O)C2(C(F)(F)F)CC2)C3)cccc1NC(c1ccc(F)cc1)C(F)(F)F] |
| I-67 | 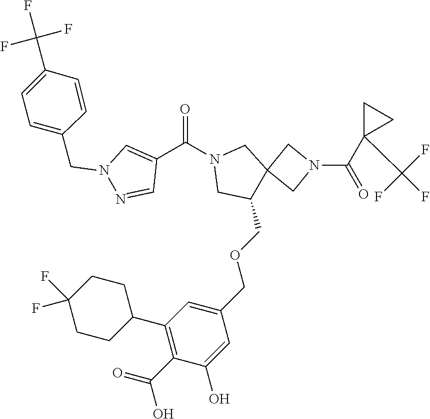 [SMILES: O=C(O)c1c(O)cc(COC[C@@H]2CN(C(=O)c3cnn(Cc4ccc(C(F)(F)F)cc4)c3)CC23CN(C(=O)C2(C(F)(F)F)CC2)C3)cc1C1CCC(F)(F)CC1] |
| I-68 | 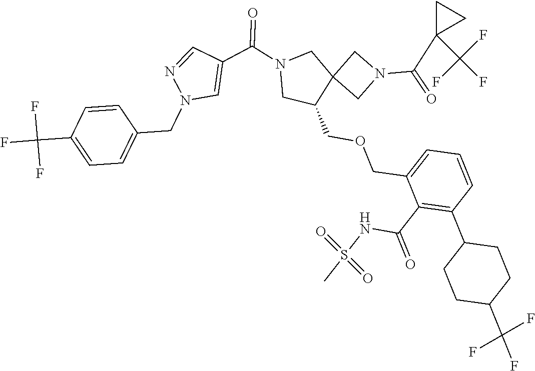 [SMILES: CS(=O)(=O)NC(=O)c1c(COC[C@@H]2CN(C(=O)c3cnn(Cc4ccc(C(F)(F)F)cc4)c3)CC23CN(C(=O)C2(C(F)(F)F)CC2)C3)cccc1C1CCC(C(F)(F)F)CC1] |
| I-69 | 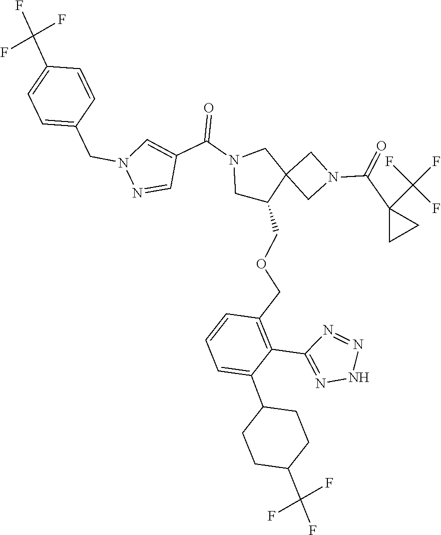 [SMILES: O=C(c1cnn(Cc2ccc(C(F)(F)F)cc2)c1)N1C[C@@H](COCc2cccc(C3CCC(C(F)(F)F)CC3)c2-c2nn[nH]n2)C2(C1)CN(C(=O)C1(C(F)(F)F)CC1)C2] |
| I-70 | 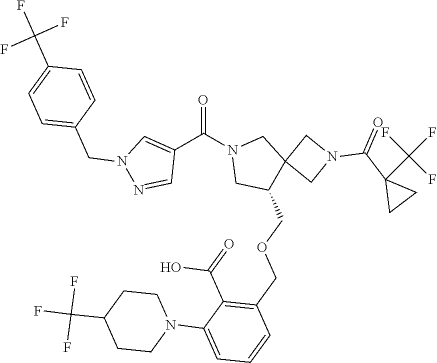 [SMILES: O=C(O)c1c(COC[C@@H]2CN(C(=O)c3cnn(Cc4ccc(C(F)(F)F)cc4)c3)CC23CN(C(=O)C2(C(F)(F)F)CC2)C3)cccc1N1CCC(C(F)(F)F)CC1] |
| I-71 | 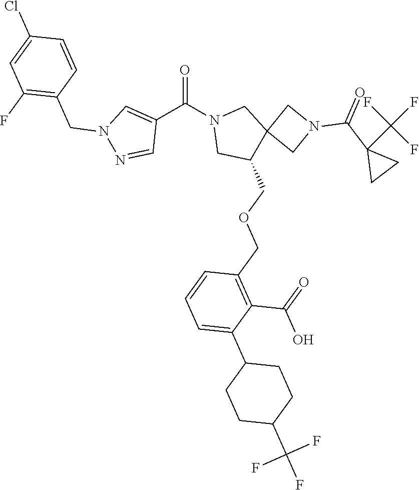 [SMILES: O=C(O)c1c(COC[C@@H]2CN(C(=O)c3cnn(Cc4ccc(Cl)cc4F)c3)CC23CN(C(=O)C2(C(F)(F)F)CC2)C3)cccc1C1CCC(C(F)(F)F)CC1] |
| I-72 | 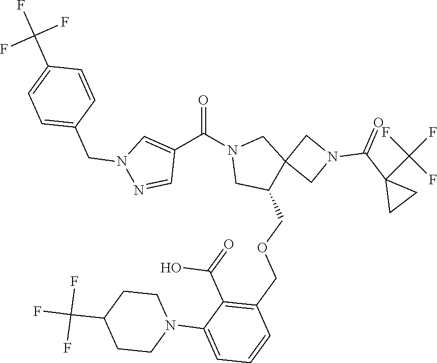 [SMILES: O=C(O)c1c(COC[C@@H]2CN(C(=O)c3cnn(Cc4ccc(C(F)(F)F)cc4)c3)CC23CN(C(=O)C2(C(F)(F)F)CC2)C3)cccc1N1CCC(C(F)(F)F)CC1] |
| I-73 | 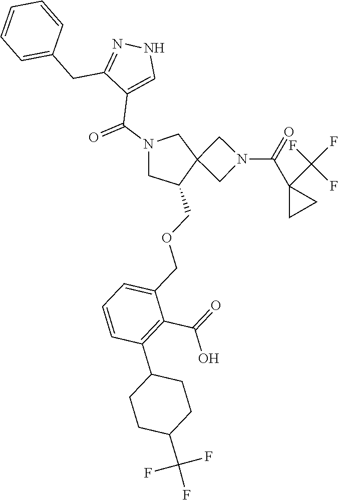 [SMILES: O=C(O)c1c(COC[C@@H]2CN(C(=O)c3c[nH]nc3Cc3ccccc3)CC23CN(C(=O)C2(C(F)(F)F)CC2)C3)cccc1C1CCC(C(F)(F)F)CC1] |
| I-74 | 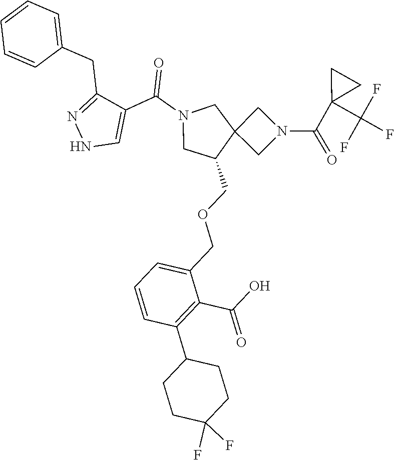 [SMILES: O=C(O)c1c(COC[C@@H]2CN(C(=O)c3c[nH]nc3Cc3ccccc3)CC23CN(C(=O)C2(C(F)(F)F)CC2)C3)cccc1C1CCC(F)(F)CC1] |
| I-75 | 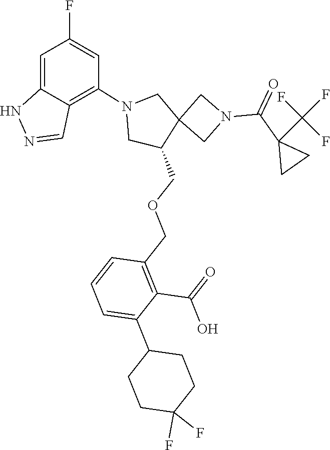 [SMILES: O=C(O)c1c(COC[C@@H]2CN(c3cc(F)cc4[nH]ncc34)CC23CN(C(=O)C2(C(F)(F)F)CC2)C3)cccc1C1CCC(F)(F)CC1] |
| I-76 | 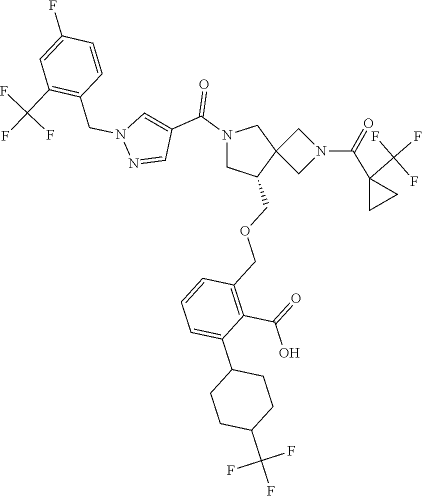 [SMILES: O=C(O)c1c(COC[C@@H]2CN(C(=O)c3cnn(Cc4ccc(F)cc4C(F)(F)F)c3)CC23CN(C(=O)C2(C(F)(F)F)CC2)C3)cccc1C1CCC(C(F)(F)F)CC1] |
| I-77 | 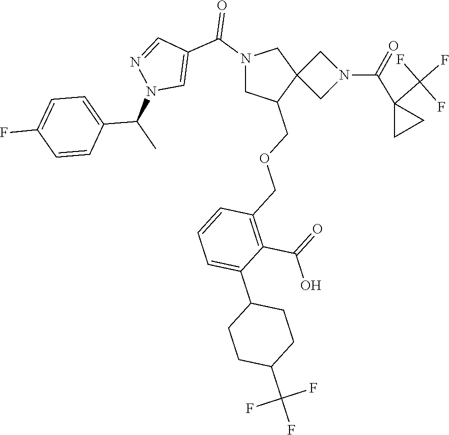 [SMILES: C[C@@H](c1ccc(F)cc1)n1cc(C(=O)N2CC(COCc3cccc(C4CCC(C(F)(F)F)CC4)c3C(=O)O)C3(C2)CN(C(=O)C2(C(F)(F)F)CC2)C3)cn1] |
| I-78 | 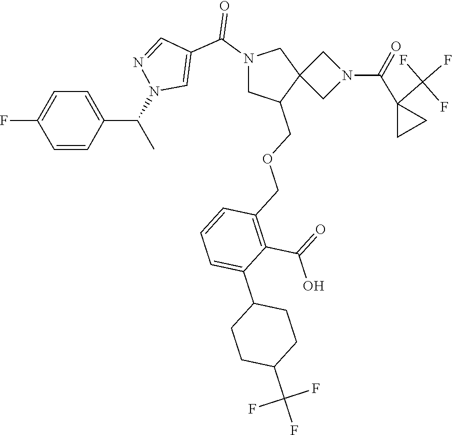 [SMILES: C[C@H](c1ccc(F)cc1)n1cc(C(=O)N2CC(COCc3cccc(C4CCC(C(F)(F)F)CC4)c3C(=O)O)C3(C2)CN(C(=O)C2(C(F)(F)F)CC2)C3)cn1] |
| I-79 | 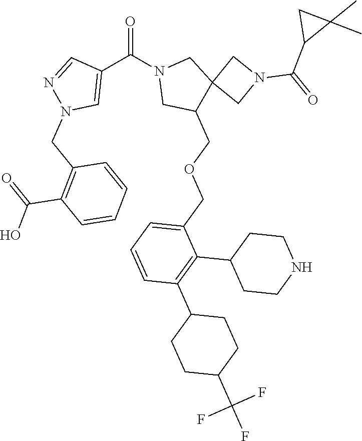 [SMILES: CC1(C)CC1C(=O)N1CC2(CN(C(=O)c3cnn(Cc4ccccc4C(=O)O)c3)CC2COCc2cccc(C3CCC(C(F)(F)F)CC3)c2C2CCNCC2)C1] |
| I-80 | 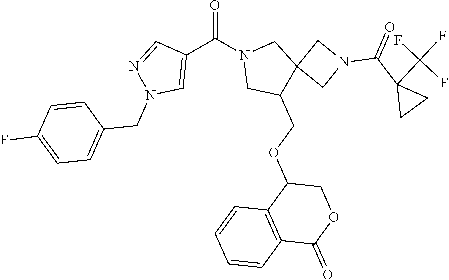 [SMILES: O=C1OCC(OCC2CN(C(=O)c3cnn(Cc4ccc(F)cc4)c3)CC23CN(C(=O)C2(C(F)(F)F)CC2)C3)c2ccccc21] |
| I-81 | 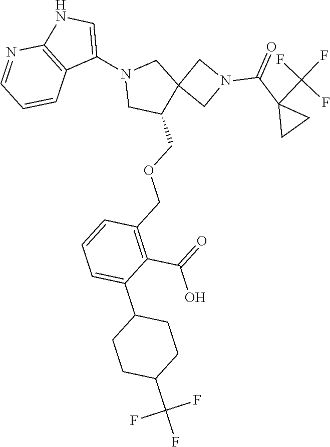 [SMILES: O=C(O)c1c(COC[C@@H]2CN(c3c[nH]c4ncccc34)CC23CN(C(=O)C2(C(F)(F)F)CC2)C3)cccc1C1CCC(C(F)(F)F)CC1] |
| I-82 | 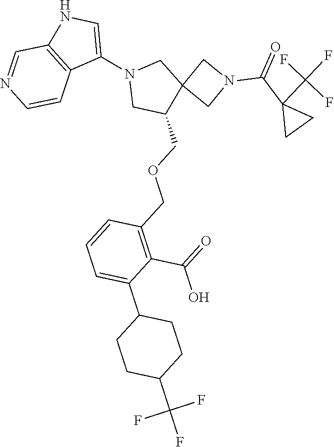 [SMILES: O=C(O)c1c(COC[C@@H]2CN(c3c[nH]c4cnccc34)CC23CN(C(=O)C2(C(F)(F)F)CC2)C3)cccc1C1CCC(C(F)(F)F)CC1] |
| I-83 | 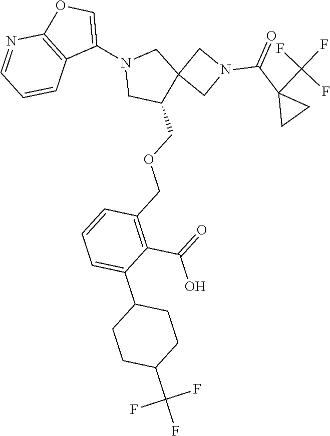 [SMILES: O=C(O)c1c(COC[C@@H]2CN(c3coc4ncccc34)CC23CN(C(=O)C2(C(F)(F)F)CC2)C3)cccc1C1CCC(C(F)(F)F)CC1] |
| I-84 | 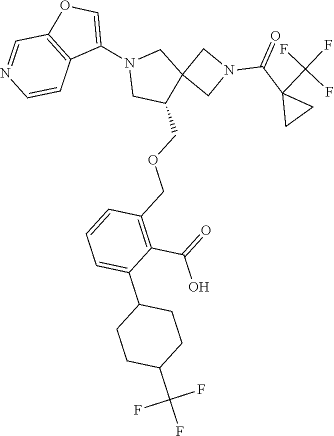 [SMILES: O=C(O)c1c(COC[C@@H]2CN(c3coc4cnccc34)CC23CN(C(=O)C2(C(F)(F)F)CC2)C3)cccc1C1CCC(C(F)(F)F)CC1] |
| I-85 | 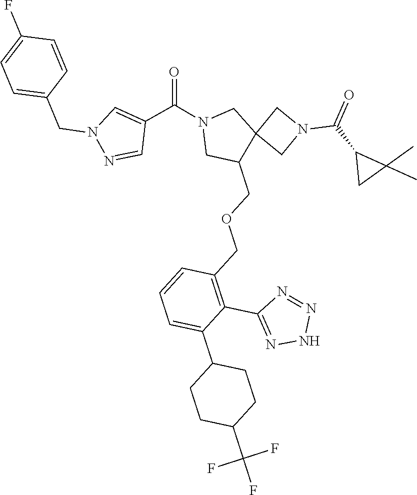 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2-c2nn[nH]n2)C1] |
| I-86 | 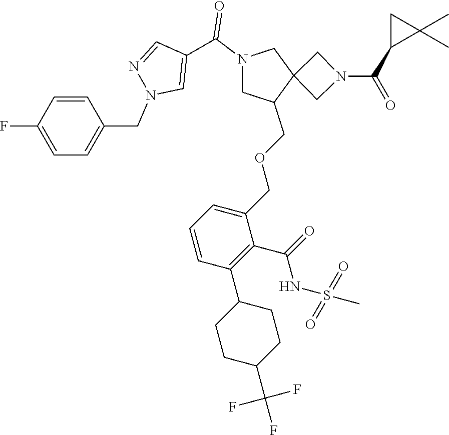 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(=O)NS(C)(=O)=O)C1] |
| I-87 | 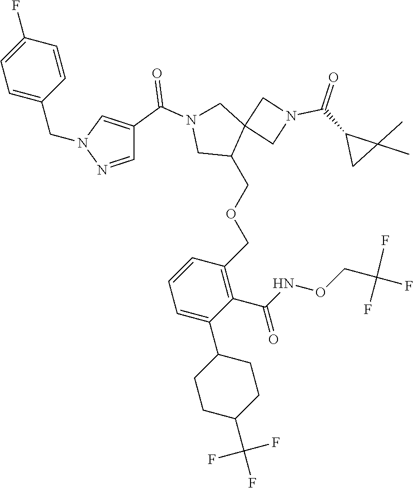 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(=O)NOCC(F)(F)F)C1] |
| I-88 | 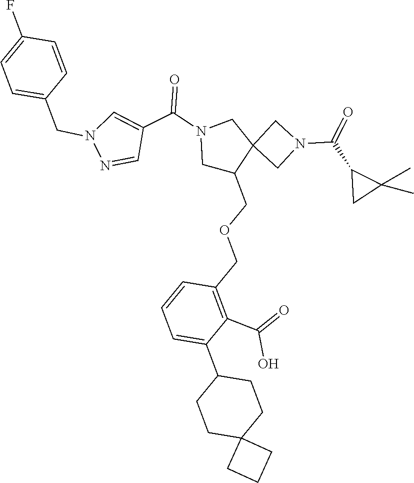 [SMILES: CC1(C)C[C@@H]1C(=O)N1CC2(CN(C(=O)c3cnn(Cc4ccc(F)cc4)c3)CC2COCc2cccc(C3CCC4(CCC4)CC3)c2C(=O)O)C1] |
| I-89 | 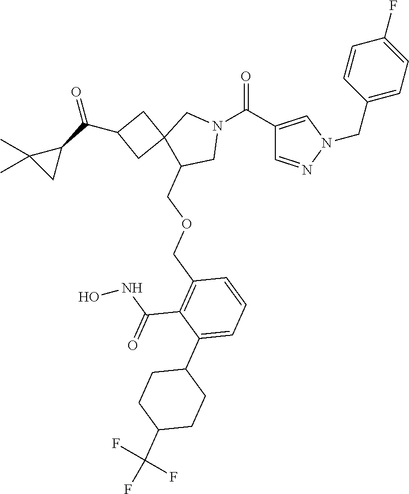 [SMILES: CC1(C)C[C@@H]1C(=O)C1CC2(C1)CN(C(=O)c1cnn(Cc3ccc(F)cc3)c1)CC2COCc1cccc(C2CCC(C(F)(F)F)CC2)c1C(=O)NO] |
| I-90 | 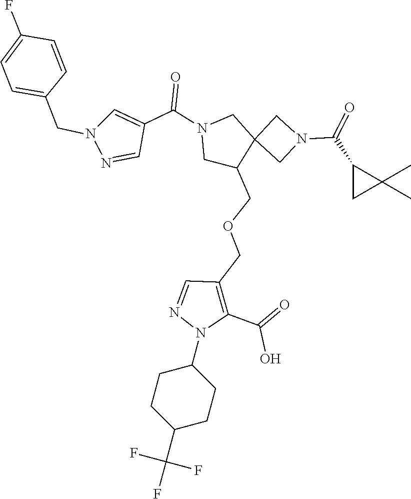 [SMILES: CC1(C)C[C@@H]1C(=O)N1CC2(CN(C(=O)c3cnn(Cc4ccc(F)cc4)c3)CC2COCc2cnn(C3CCC(C(F)(F)F)CC3)c2C(=O)O)C1] |
| I-91 | 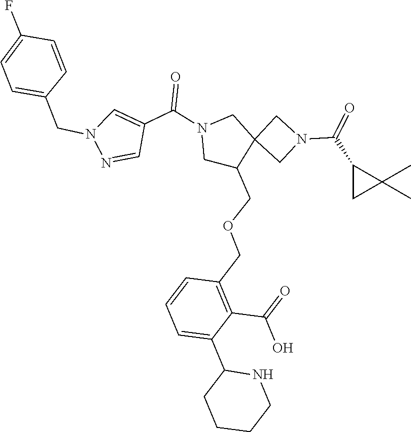 [SMILES: CC1(C)C[C@@H]1C(=O)N1CC2(CN(C(=O)c3cnn(Cc4ccc(F)cc4)c3)CC2COCc2cccc(C3CCCCN3)c2C(=O)O)C1] |
| I-92 | 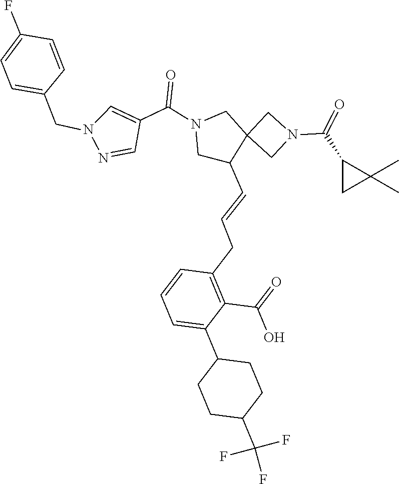 [SMILES: CC1(C)C[C@@H]1C(=O)N1CC2(CN(C(=O)c3cnn(Cc4ccc(F)cc4)c3)CC2/C=C/Cc2cccc(C3CCC(C(F)(F)F)CC3)c2C(=O)O)C1] |
| I-93 | 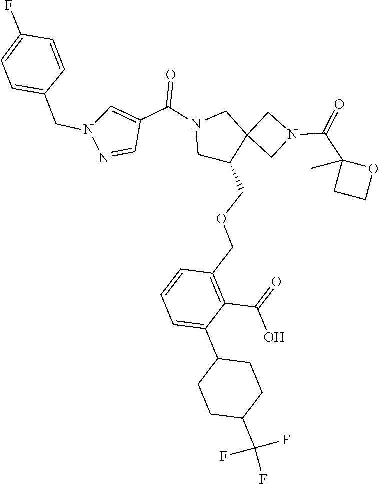 [SMILES: CC1(C(=O)N2CC3(CN(C(=O)c4cnn(Cc5ccc(F)cc5)c4)C[C@H]3COCc3cccc(C4CCC(C(F)(F)F)CC4)c3C(=O)O)C2)CCO1] |
| I-94 | 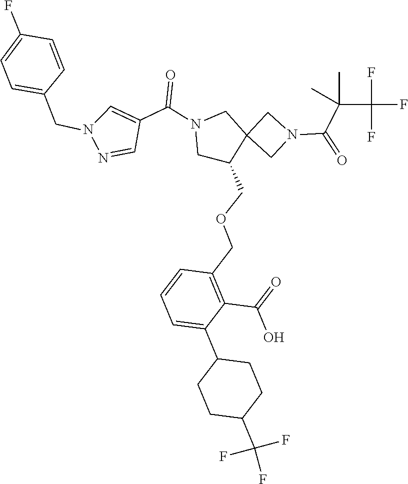 [SMILES: CC(C)(C(=O)N1CC2(CN(C(=O)c3cnn(Cc4ccc(F)cc4)c3)C[C@H]2COCc2cccc(C3CCC(C(F)(F)F)CC3)c2C(=O)O)C1)C(F)(F)F] |
| I-95 | 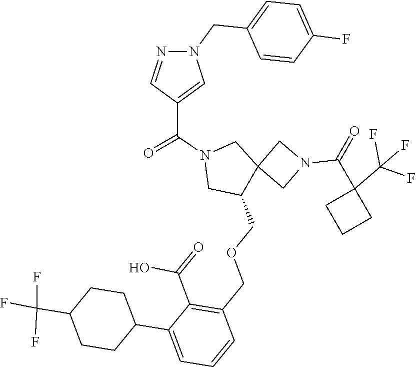 [SMILES: O=C(O)c1c(COC[C@@H]2CN(C(=O)c3cnn(Cc4ccc(F)cc4)c3)CC23CN(C(=O)C2(C(F)(F)F)CCC2)C3)cccc1C1CCC(C(F)(F)F)CC1] |
| I-96 | 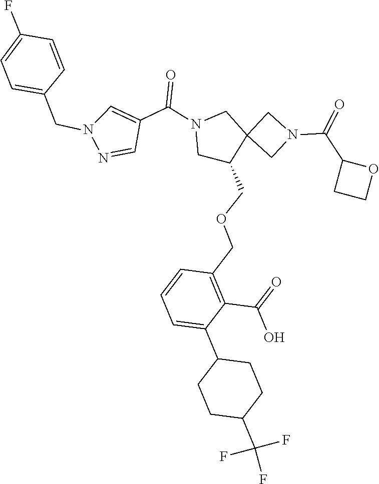 [SMILES: O=C(O)c1c(COC[C@@H]2CN(C(=O)c3cnn(Cc4ccc(F)cc4)c3)CC23CN(C(=O)C2CCO2)C3)cccc1C1CCC(C(F)(F)F)CC1] |
| I-97 | 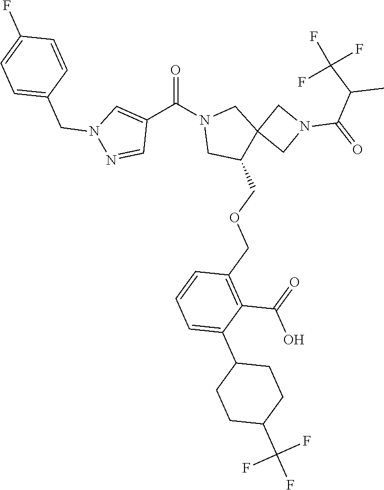 [SMILES: CC(C(=O)N1CC2(CN(C(=O)c3cnn(Cc4ccc(F)cc4)c3)C[C@H]2COCc2cccc(C3CCC(C(F)(F)F)CC3)c2C(=O)O)C1)C(F)(F)F] |
| I-98 | 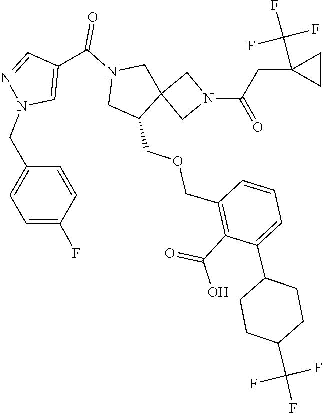 [SMILES: O=C(O)c1c(COC[C@@H]2CN(C(=O)c3cnn(Cc4ccc(F)cc4)c3)CC23CN(C(=O)CC2(C(F)(F)F)CC2)C3)cccc1C1CCC(C(F)(F)F)CC1] |
| I-99 | 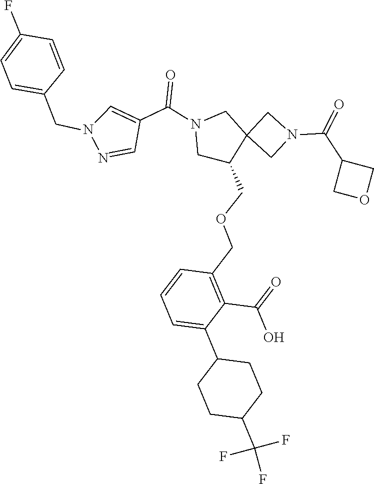 [SMILES: O=C(O)c1c(COC[C@@H]2CN(C(=O)c3cnn(Cc4ccc(F)cc4)c3)CC23CN(C(=O)C2COC2)C3)cccc1C1CCC(C(F)(F)F)CC1] |
| I-100 | 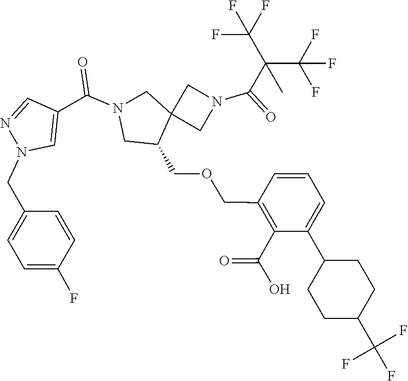 [SMILES: CC(C(=O)N1CC2(CN(C(=O)c3cnn(Cc4ccc(F)cc4)c3)C[C@H]2COCc2cccc(C3CCC(C(F)(F)F)CC3)c2C(=O)O)C1)(C(F)(F)F)C(F)(F)F] |
| I-101 | 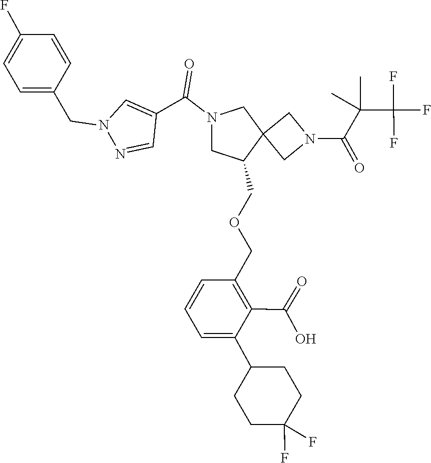 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)O)C1)C(F)(F)F] |
| I-102 | 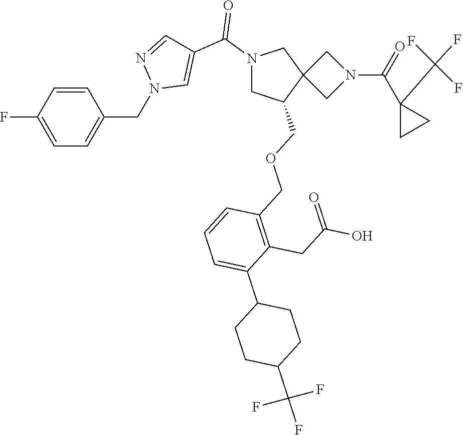 [SMILES: O=C(O)Cc1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-103 | 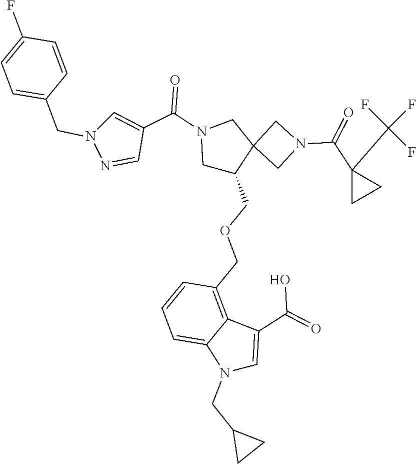 [SMILES: O=C(O)c1cn(CC2CC2)c2cccc(COC[C@@H]3CN(C(=O)c4cnn(Cc5ccc(F)cc5)c4)CC34CN(C(=O)C3(C(F)(F)F)CC3)C4)c12] |
| I-104 | 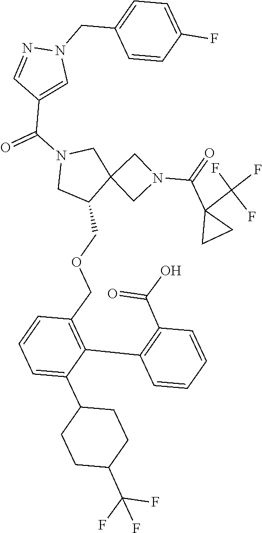 [SMILES: O=C(O)c1ccccc1-c1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-105 | 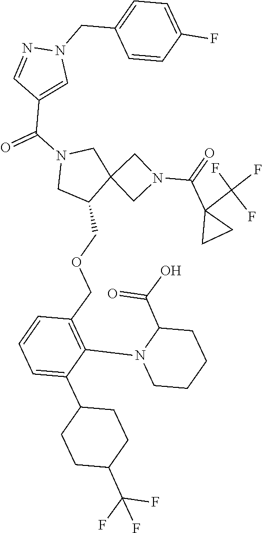 [SMILES: O=C(O)C1CCCCN1c1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-106 | 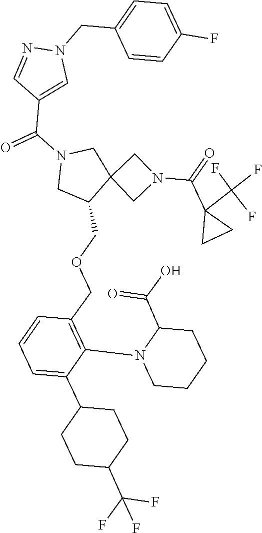 [SMILES: O=C(O)C1CCCCN1c1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-107 | 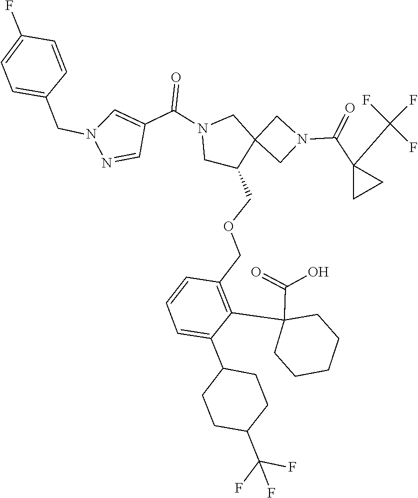 [SMILES: O=C(c1cnn(Cc2ccc(F)cc2)c1)N1C[C@@H](COCc2cccc(C3CCC(C(F)(F)F)CC3)c2C2(C(=O)O)CCCCC2)C2(C1)CN(C(=O)C1(C(F)(F)F)CC1)C2] |
| I-108 | 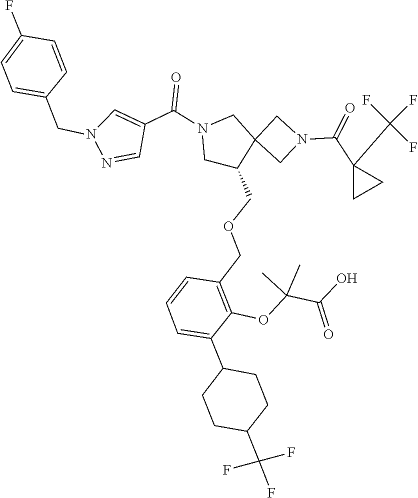 [SMILES: CC(C)(Oc1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1)C(=O)O] |
| I-109 | 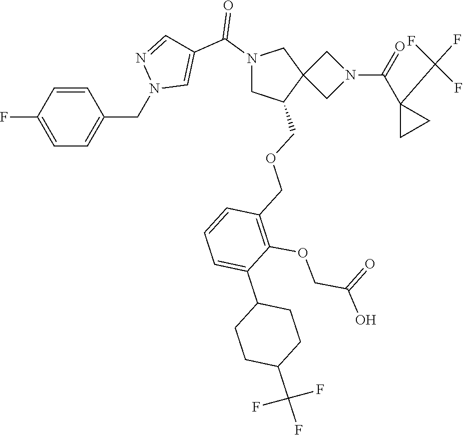 [SMILES: O=C(O)COc1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-110 | 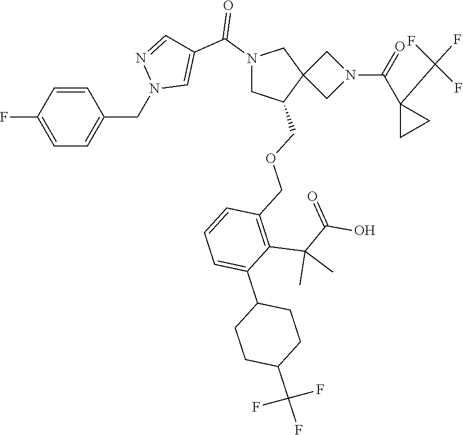 [SMILES: CC(C)(C(=O)O)c1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-111 | 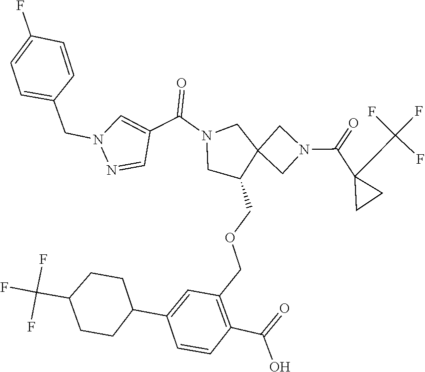 [SMILES: O=C(O)c1ccc(C2CCC(C(F)(F)F)CC2)cc1COC[C@@H]1CN(C(=O)c2cnn(Cc3ccc(F)cc3)c2)CC12CN(C(=O)C1(C(F)(F)F)CC1)C2] |
| I-112 | 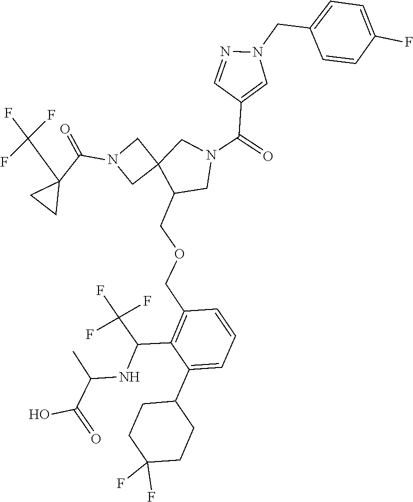 [SMILES: CC(NC(c1c(COCC2CN(C(=O)c3cnn(Cc4ccc(F)cc4)c3)CC23CN(C(=O)C2(C(F)(F)F)CC2)C3)cccc1C1CCC(F)(F)CC1)C(F)(F)F)C(=O)O] |
| I-113 | 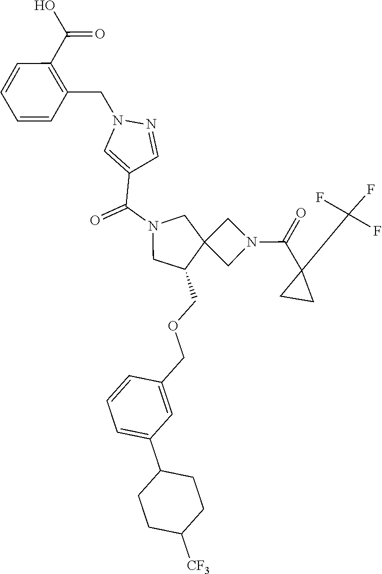 [SMILES: O=C(O)c1ccccc1Cn1cc(C(=O)N2C[C@@H](COCc3cccc(C4CCC(C(F)(F)F)CC4)c3)C3(C2)CN(C(=O)C2(C(F)(F)F)CC2)C3)cn1] |
| I-114 | 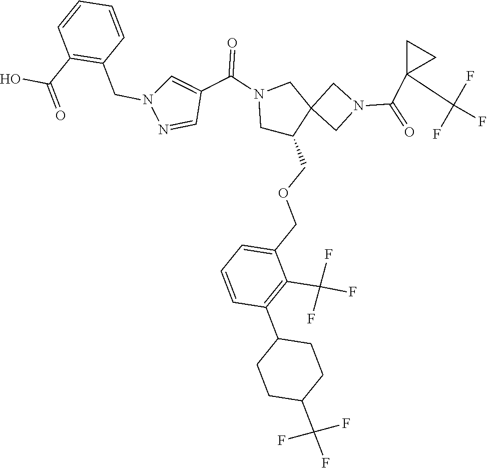 |
| I-115 | 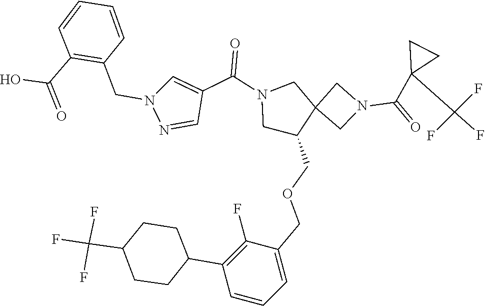 [SMILES: O=C(O)c1ccccc1Cn1cc(C(=O)N2C[C@@H](COCc3cccc(C4CCC(C(F)(F)F)CC4)c3F)C3(C2)CN(C(=O)C2(C(F)(F)F)CC2)C3)cn1] |
| I-116 | 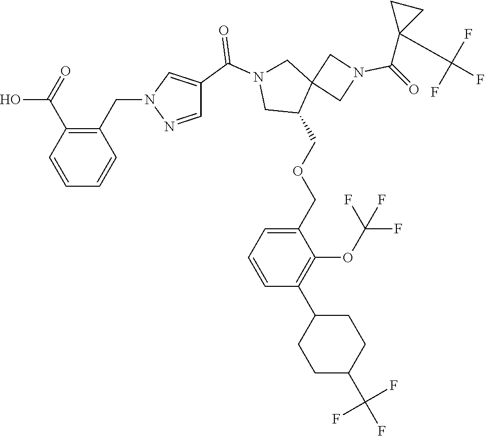 |
| I-117 | 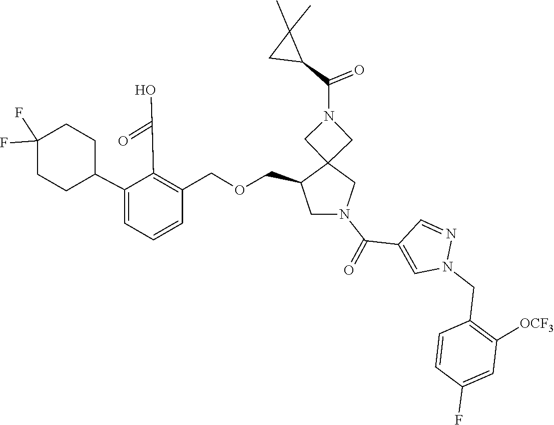 [SMILES: CC1(C)C[C@@H]1C(=O)N1CC2(CN(C(=O)c3cnn(Cc4ccc(F)cc4OC(F)(F)F)c3)C[C@H]2COCc2cccc(C3CCC(F)(F)CC3)c2C(=O)O)C1] |
| I-118 | 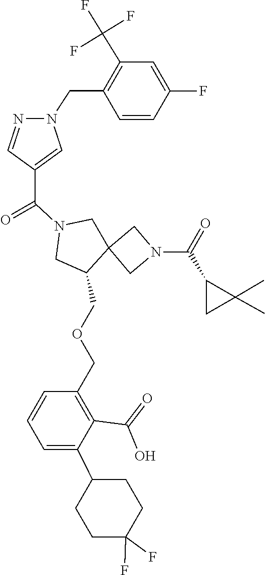 [SMILES: CC1(C)C[C@@H]1C(=O)N1CC2(CN(C(=O)c3cnn(Cc4ccc(F)cc4C(F)(F)F)c3)C[C@H]2COCc2cccc(C3CCC(F)(F)CC3)c2C(=O)O)C1] |
| I-119 | 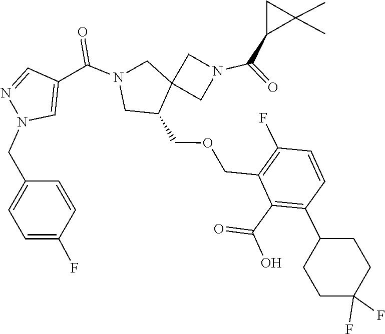 [SMILES: CC1(C)C[C@@H]1C(=O)N1CC2(CN(C(=O)c3cnn(Cc4ccc(F)cc4)c3)C[C@H]2COCc2c(F)ccc(C3CCC(F)(F)CC3)c2C(=O)O)C1] |
| I-120 | 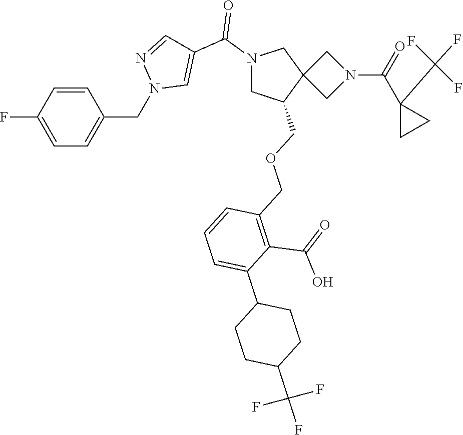 [SMILES: O=C(O)c1c(COC[C@@H]2CN(C(=O)c3cnn(Cc4ccc(F)cc4)c3)CC23CN(C(=O)C2(C(F)(F)F)CC2)C3)cccc1C1CCC(C(F)(F)F)CC1] |
| I-121 | 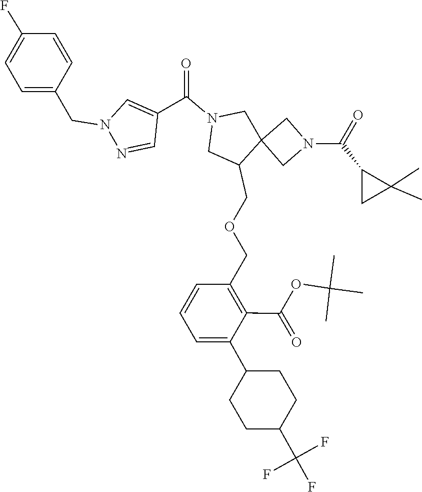 [SMILES: CC(C)(C)OC(=O)c1c(COCC2CN(C(=O)c3cnn(Cc4ccc(F)cc4)c3)CC23CN(C(=O)[C@H]2CC2(C)C)C3)cccc1C1CCC(C(F)(F)F)CC1] |
| I-122 | 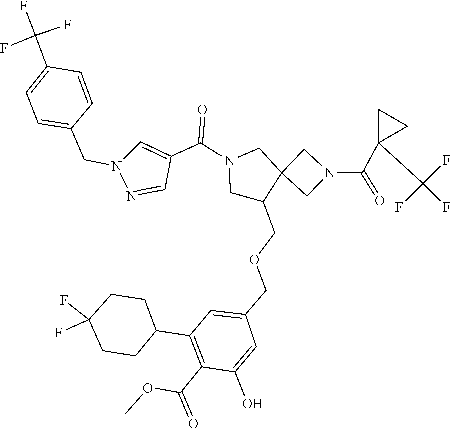 [SMILES: COC(=O)c1c(O)cc(COCC2CN(C(=O)c3cnn(Cc4ccc(C(F)(F)F)cc4)c3)CC23CN(C(=O)C2(C(F)(F)F)CC2)C3)cc1C1CCC(F)(F)CC1] |
| I-123 | 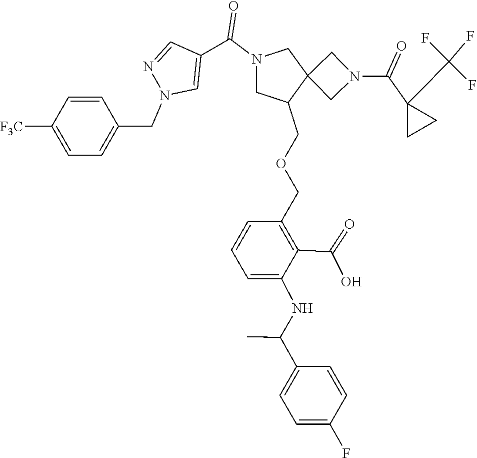 [SMILES: CC(Nc1cccc(COCC2CN(C(=O)c3cnn(Cc4ccc(C(F)(F)F)cc4)c3)CC23CN(C(=O)C2(C(F)(F)F)CC2)C3)c1C(=O)O)c1ccc(F)cc1] |
| I-124 | 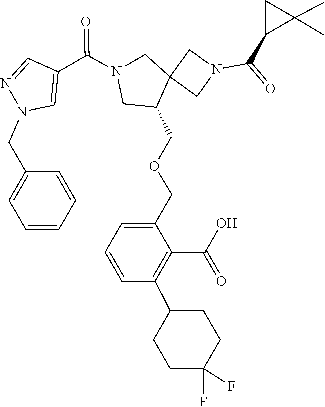 [SMILES: CC1(C)C[C@@H]1C(=O)N1CC2(CN(C(=O)c3cnn(Cc4ccccc4)c3)C[C@H]2COCc2cccc(C3CCC(F)(F)CC3)c2C(=O)O)C1] |
| I-125 | 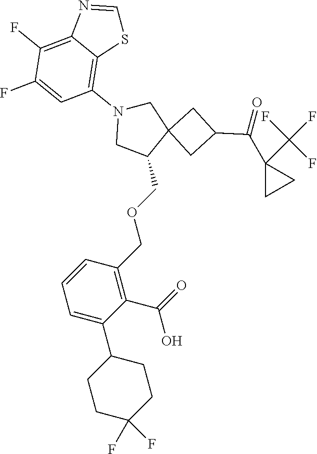 [SMILES: O=C(O)c1c(COC[C@@H]2CN(c3cc(F)c(F)c4ncsc34)CC23CC(C(=O)C2(C(F)(F)F)CC2)C3)cccc1C1CCC(F)(F)CC1] |
| I-126 | 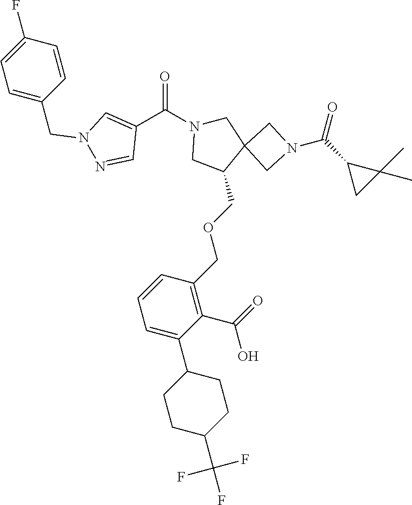 [SMILES: CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2C(=O)O)C1] |
| I-127 | 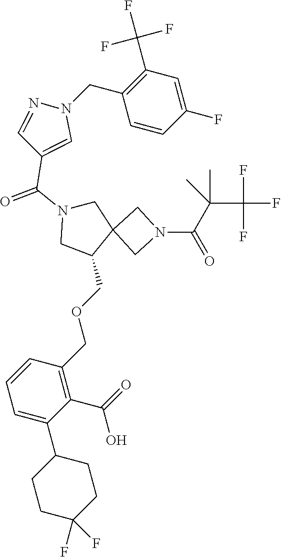 [SMILES: CC(C)(C(=O)N1CC2(CN(C(=O)c3cnn(Cc4ccc(F)cc4C(F)(F)F)c3)C[C@H]2COCc2cccc(C3CCC(F)(F)CC3)c2C(=O)O)C1)C(F)(F)F] |
| I-128 | 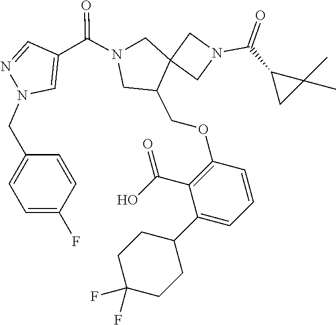 [SMILES: CC1(C)C[C@@H]1C(=O)N1CC2(CN(C(=O)c3cnn(Cc4ccc(F)cc4)c3)CC2COc2cccc(C3CCC(F)(F)CC3)c2C(=O)O)C1] |
| I-129 | 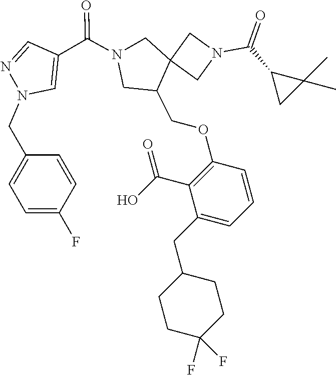 [SMILES: CC1(C)C[C@@H]1C(=O)N1CC2(CN(C(=O)c3cnn(Cc4ccc(F)cc4)c3)CC2COc2cccc(CC3CCC(F)(F)CC3)c2C(=O)O)C1] |
| I-130 | 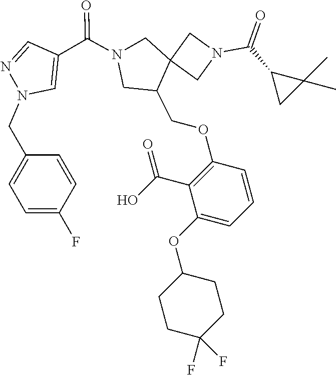 [SMILES: CC1(C)C[C@@H]1C(=O)N1CC2(CN(C(=O)c3cnn(Cc4ccc(F)cc4)c3)CC2COc2cccc(OC3CCC(F)(F)CC3)c2C(=O)O)C1] |

TABLE 8B
Exemplary Compounds

| Compound | |
|---|---|
| No. | Structure |
| I′-1 | 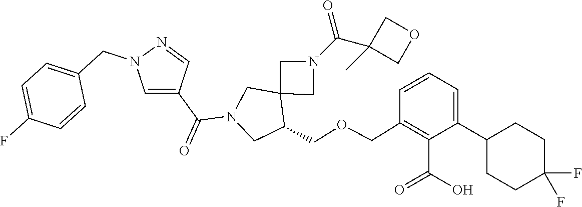 [SMILES: CC1(C(=O)N2CC3(CN(C(=O)c4cnn(Cc5ccc(F)cc5)c4)C[C@H]3COCc3cccc(C4CCC(F)(F)CC4)c3C(=O)O)C2)COC1] |
| I′-2 | 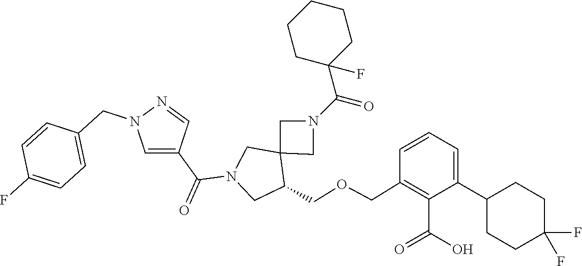 [SMILES: O=C(O)c1c(COC[C@@H]2CN(C(=O)c3cnn(Cc4ccc(F)cc4)c3)CC23CN(C(=O)C2(F)CCCCC2)C3)cccc1C1CCC(F)(F)CC1] |
| I′-3 | 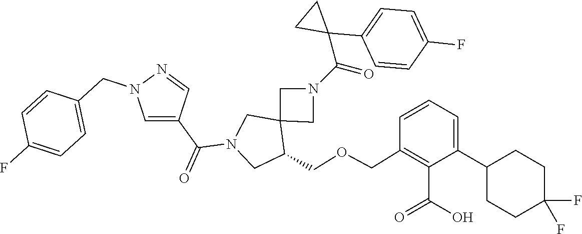 [SMILES: O=C(O)c1c(COC[C@@H]2CN(C(=O)c3cnn(Cc4ccc(F)cc4)c3)CC23CN(C(=O)C2(c4ccc(F)cc4)CC2)C3)cccc1C1CCC(F)(F)CC1] |
| I′-4 | 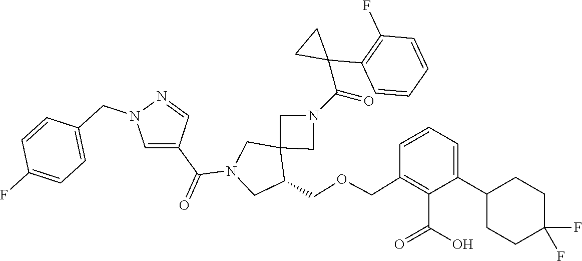 [SMILES: O=C(O)c1c(COC[C@@H]2CN(C(=O)c3cnn(Cc4ccc(F)cc4)c3)CC23CN(C(=O)C2(c4ccccc4F)CC2)C3)cccc1C1CCC(F)(F)CC1] |
| I′-5 | 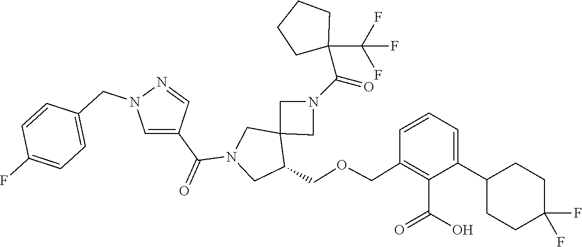 [SMILES: O=C(O)c1c(COC[C@@H]2CN(C(=O)c3cnn(Cc4ccc(F)cc4)c3)CC23CN(C(=O)C2(C(F)(F)F)CCCC2)C3)cccc1C1CCC(F)(F)CC1] |
| I′-6 | 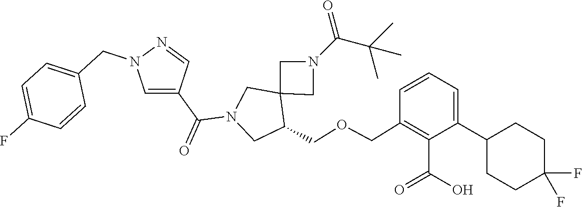 [SMILES: CC(C)(C)C(=O)N1CC2(CN(C(=O)c3cnn(Cc4ccc(F)cc4)c3)C[C@H]2COCc2cccc(C3CCC(F)(F)CC3)c2C(=O)O)C1] |
| I′-7 | 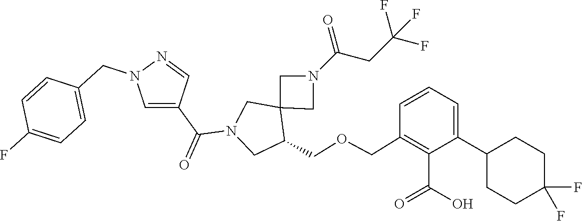 [SMILES: O=C(O)c1c(COC[C@@H]2CN(C(=O)c3cnn(Cc4ccc(F)cc4)c3)CC23CN(C(=O)CC(F)(F)F)C3)cccc1C1CCC(F)(F)CC1] |
| I′-8 | 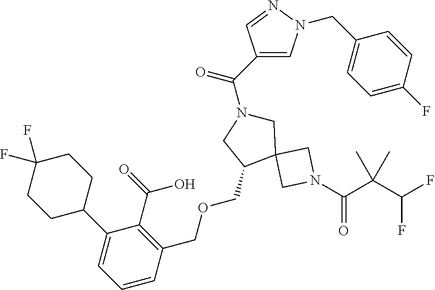 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)O)C1)C(F)F] |
| I′-9 | 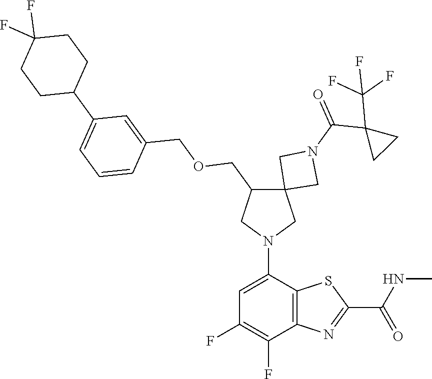 [SMILES: CNC(=O)c1nc2c(F)c(F)cc(N3CC(COCc4cccc(C5CCC(F)(F)CC5)c4)C4(CN(C(=O)C5(C(F)(F)F)CC5)C4)C3)c2s1] |
| I′-10 | 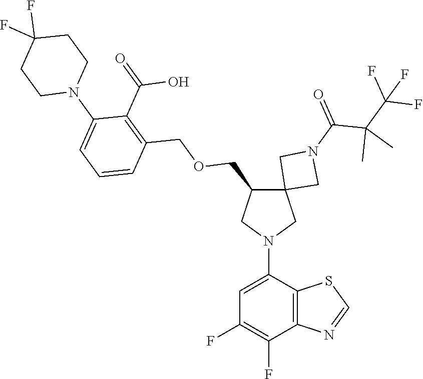 [SMILES: CC(C)(C(=O)N1CC2(C1)CN(c1cc(F)c(F)c3ncsc13)C[C@H]2COCc1cccc(N2CCC(F)(F)CC2)c1C(=O)O)C(F)(F)F] |
| I′-11 | 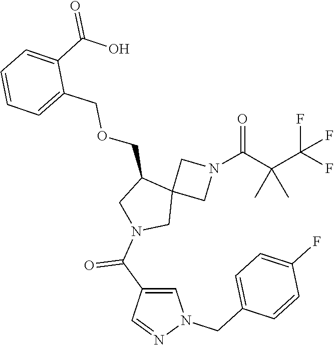 [SMILES: CC(C)(C(=O)N1CC2(CN(C(=O)c3cnn(Cc4ccc(F)cc4)c3)C[C@H]2COCc2ccccc2C(=O)O)C1)C(F)(F)F] |
| I′-12 | 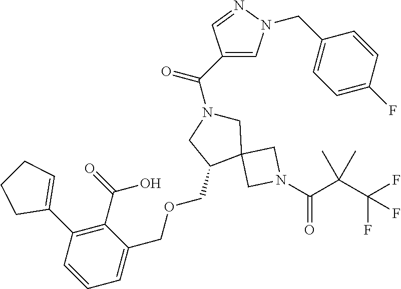 [SMILES: CC(C)(C(=O)N1CC2(CN(C(=O)c3cnn(Cc4ccc(F)cc4)c3)C[C@H]2COCc2cccc(C3=CCCC3)c2C(=O)O)C1)C(F)(F)F] |
| I′-13 | 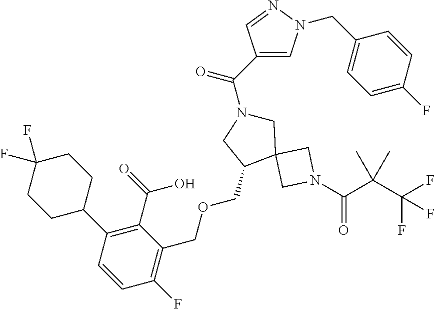 [SMILES: CC(C)(C(=O)N1CC2(CN(C(=O)c3cnn(Cc4ccc(F)cc4)c3)C[C@H]2COCc2c(F)ccc(C3CCC(F)(F)CC3)c2C(=O)O)C1)C(F)(F)F] |
| I′-14 | 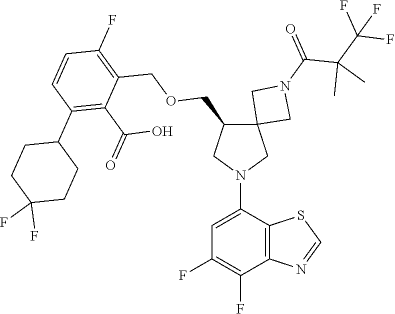 [SMILES: CC(C)(C(=O)N1CC2(C1)CN(c1cc(F)c(F)c3ncsc13)C[C@H]2COCc1c(F)ccc(C2CCC(F)(F)CC2)c1C(=O)O)C(F)(F)F] |
| I′-15 | 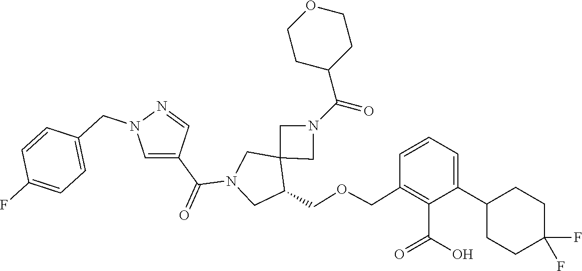 [SMILES: O=C(O)c1c(COC[C@@H]2CN(C(=O)c3cnn(Cc4ccc(F)cc4)c3)CC23CN(C(=O)C2CCOCC2)C3)cccc1C1CCC(F)(F)CC1] |
| I′-16 | 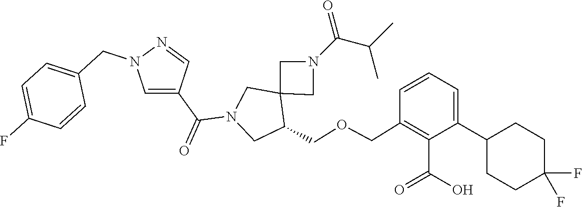 [SMILES: CC(C)C(=O)N1CC2(CN(C(=O)c3cnn(Cc4ccc(F)cc4)c3)C[C@H]2COCc2cccc(C3CCC(F)(F)CC3)c2C(=O)O)C1] |
| I′-17 | 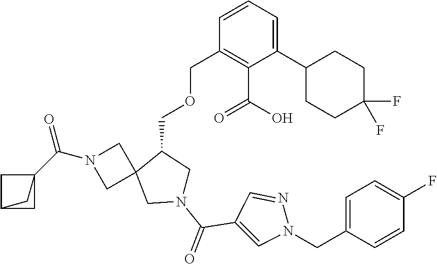 [SMILES: O=C(O)c1c(COC[C@@H]2CN(C(=O)c3cnn(Cc4ccc(F)cc4)c3)CC23CN(C(=O)C24CC(C2)C4)C3)cccc1C1CCC(F)(F)CC1] |
| I′-18 | 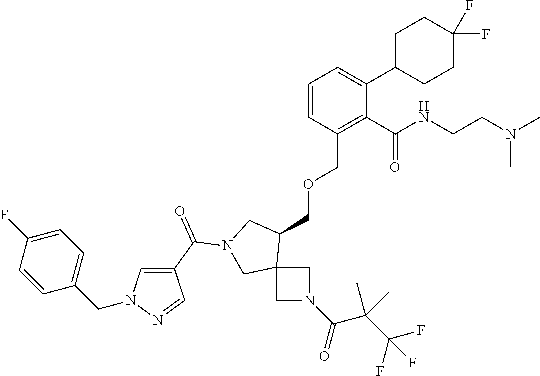 [SMILES: CN(C)CCNC(=O)c1c(COC[C@@H]2CN(C(=O)c3cnn(Cc4ccc(F)cc4)c3)CC23CN(C(=O)C(C)(C)C(F)(F)F)C3)cccc1C1CCC(F)(F)CC1] |
| I′-19 | 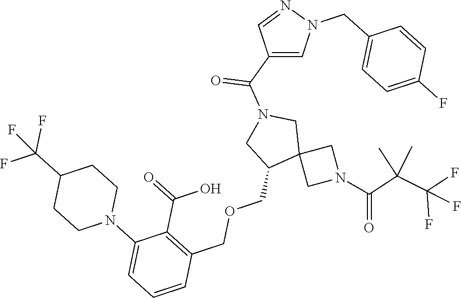 [SMILES: CC(C)(C(=O)N1CC2(CN(C(=O)c3cnn(Cc4ccc(F)cc4)c3)C[C@H]2COCc2cccc(N3CCC(C(F)(F)F)CC3)c2C(=O)O)C1)C(F)(F)F] |
| I′-20 | 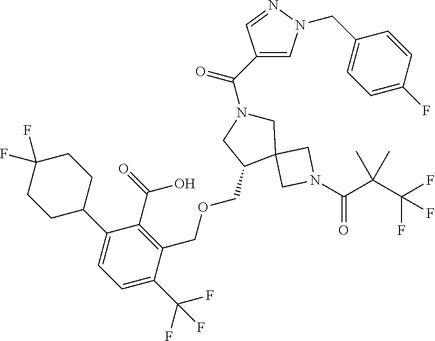 [SMILES: CC(C)(C(=O)N1CC2(CN(C(=O)c3cnn(Cc4ccc(F)cc4)c3)C[C@H]2COCc2c(C(F)(F)F)ccc(C3CCC(F)(F)CC3)c2C(=O)O)C1)C(F)(F)F] |
| I′-21 | 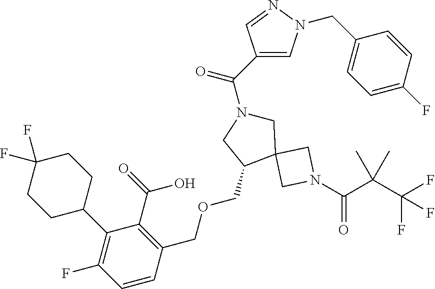 [SMILES: CC(C)(C(=O)N1CC2(CN(C(=O)c3cnn(Cc4ccc(F)cc4)c3)C[C@H]2COCc2ccc(F)c(C3CCC(F)(F)CC3)c2C(=O)O)C1)C(F)(F)F] |
| I′-22 | 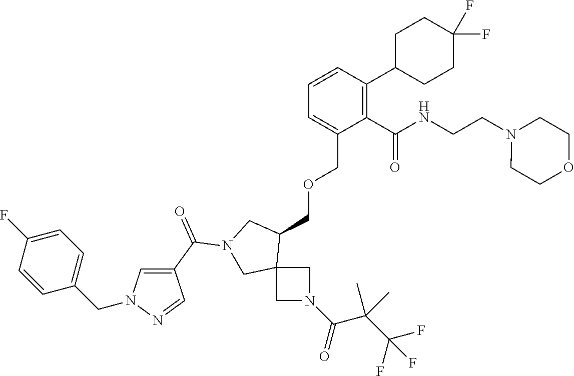 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)NCCN2CCOCC2)C1)C(F)(F)F] |
| I′-23 | 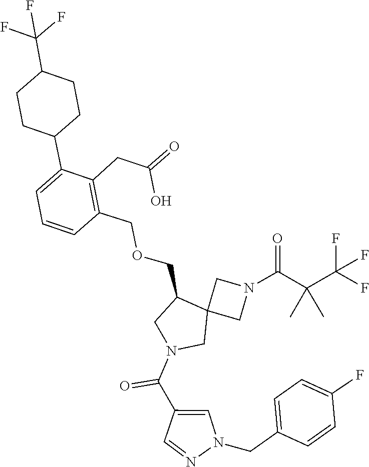 [SMILES: CC(C)(C(=O)N1CC2(CN(C(=O)c3cnn(Cc4ccc(F)cc4)c3)C[C@H]2COCc2cccc(C3CCC(C(F)(F)F)CC3)c2CC(=O)O)C1)C(F)(F)F] |
| I′-24 | 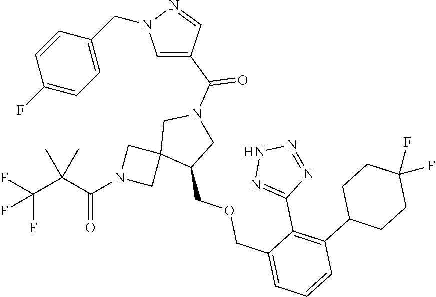 [SMILES: CC(C)(C(=O)N1CC2(CN(C(=O)c3cnn(Cc4ccc(F)cc4)c3)C[C@H]2COCc2cccc(C3CCC(F)(F)CC3)c2-c2nn[nH]n2)C1)C(F)(F)F] |
| I′-25 | 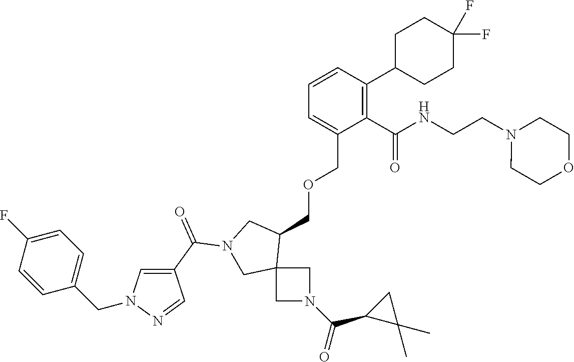 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)NCCN2CCOCC2)C1] |
| I′-26 | 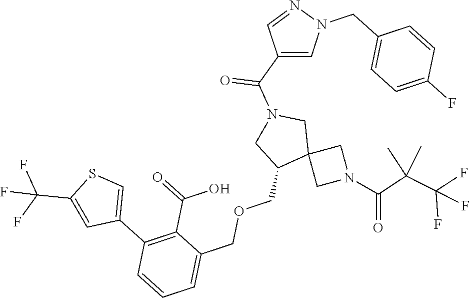 [SMILES: CC(C)(C(=O)N1CC2(CN(C(=O)c3cnn(Cc4ccc(F)cc4)c3)C[C@H]2COCc2cccc(-c3csc(C(F)(F)F)c3)c2C(=O)O)C1)C(F)(F)F] |
| I′-27 | 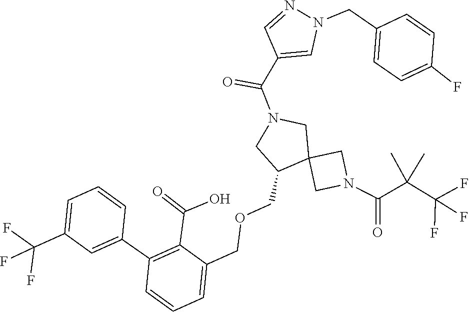 [SMILES: CC(C)(C(=O)N1CC2(CN(C(=O)c3cnn(Cc4ccc(F)cc4)c3)C[C@H]2COCc2cccc(-c3cccc(C(F)(F)F)c3)c2C(=O)O)C1)C(F)(F)F] |
| I′-28 | 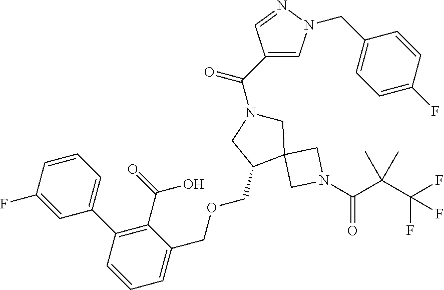 [SMILES: CC(C)(C(=O)N1CC2(CN(C(=O)c3cnn(Cc4ccc(F)cc4)c3)C[C@H]2COCc2cccc(-c3cccc(F)c3)c2C(=O)O)C1)C(F)(F)F] |
| I′-29 | 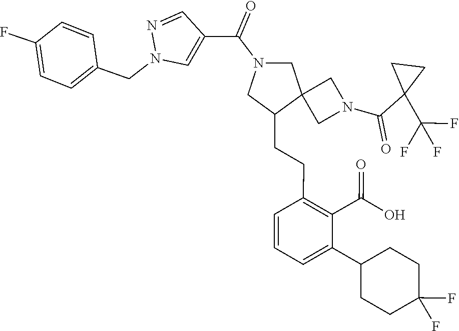 [SMILES: O=C(O)c1c(CCC2CN(C(=O)c3cnn(Cc4ccc(F)cc4)c3)CC23CN(C(=O)C2(C(F)(F)F)CC2)C3)cccc1C1CCC(F)(F)CC1] |
| I′-30 | 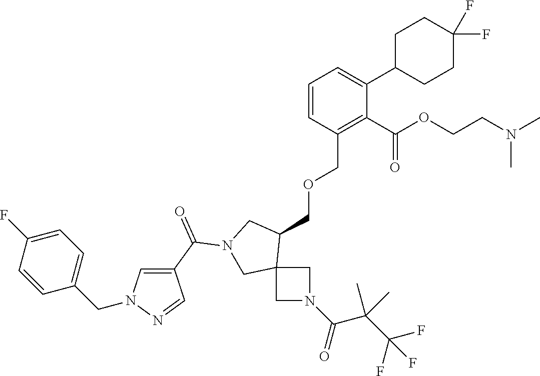 [SMILES: CN(C)CCOC(=O)c1c(COC[C@@H]2CN(C(=O)c3cnn(Cc4ccc(F)cc4)c3)CC23CN(C(=O)C(C)(C)C(F)(F)F)C3)cccc1C1CCC(F)(F)CC1] |
| I′-31 | 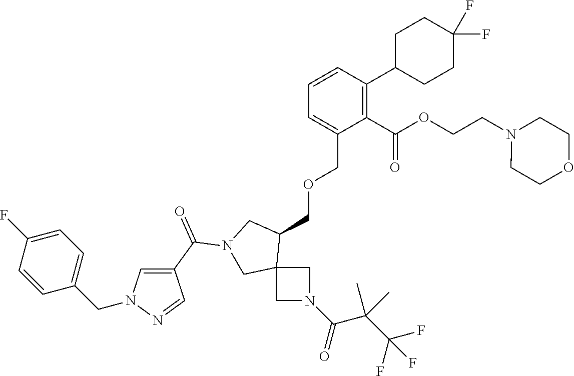 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)OCCN2CCOCC2)C1)C(F)(F)F] |
| I′-32 | 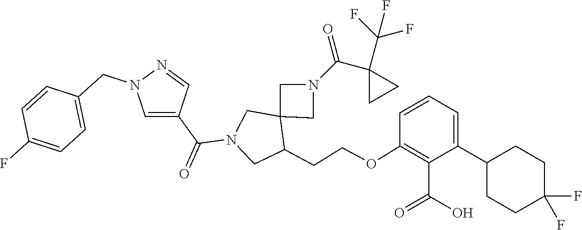 [SMILES: O=C(O)c1c(OCCC2CN(C(=O)c3cnn(Cc4ccc(F)cc4)c3)CC23CN(C(=O)C2(C(F)(F)F)CC2)C3)cccc1C1CCC(F)(F)CC1] |
| I′-33 | 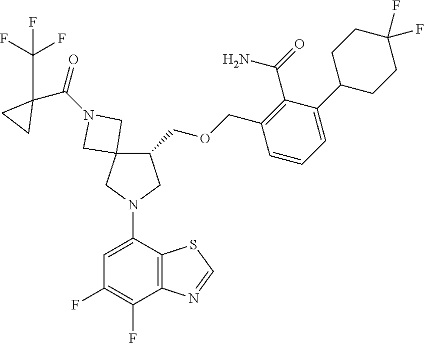 [SMILES: NC(=O)c1c(COC[C@@H]2CN(c3cc(F)c(F)c4ncsc34)CC23CN(C(=O)C2(C(F)(F)F)CC2)C3)cccc1C1CCC(F)(F)CC1] |
| I′-34 | 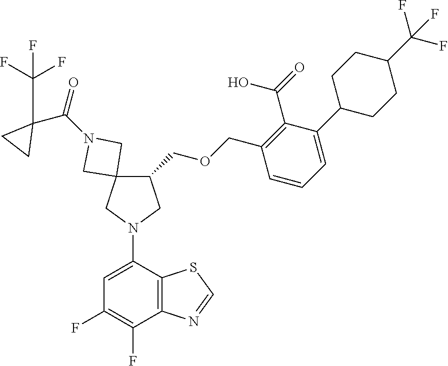 [SMILES: O=C(O)c1c(COC[C@@H]2CN(c3cc(F)c(F)c4ncsc34)CC23CN(C(=O)C2(C(F)(F)F)CC2)C3)cccc1C1CCC(C(F)(F)F)CC1] |
| I′-35 | 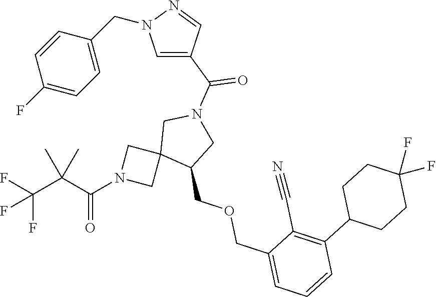 [SMILES: CC(C)(C(=O)N1CC2(CN(C(=O)c3cnn(Cc4ccc(F)cc4)c3)C[C@H]2COCc2cccc(C3CCC(F)(F)CC3)c2C#N)C1)C(F)(F)F] |
| I′-36 | 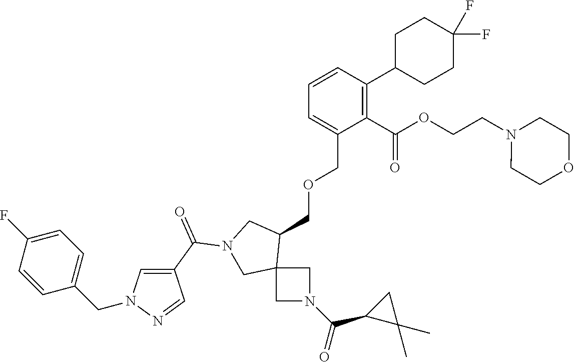 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)OCCN2CCOCC2)C1] |
| I′-37 | 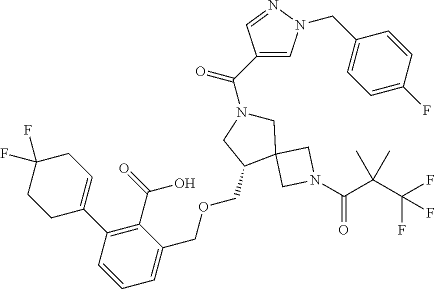 [SMILES: CC(C)(C(=O)N1CC2(CN(C(=O)c3cnn(Cc4ccc(F)cc4)c3)C[C@H]2COCc2cccc(C3=CCC(F)(F)CC3)c2C(=O)O)C1)C(F)(F)F] |
| I′-38 | 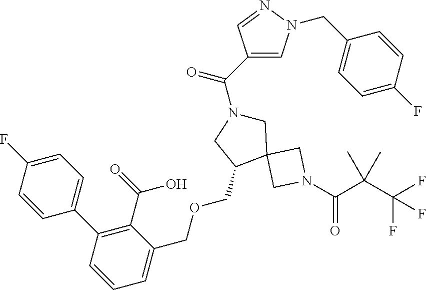 [SMILES: CC(C)(C(=O)N1CC2(CN(C(=O)c3cnn(Cc4ccc(F)cc4)c3)C[C@H]2COCc2cccc(-c3ccc(F)cc3)c2C(=O)O)C1)C(F)(F)F] |
| I′-39 | 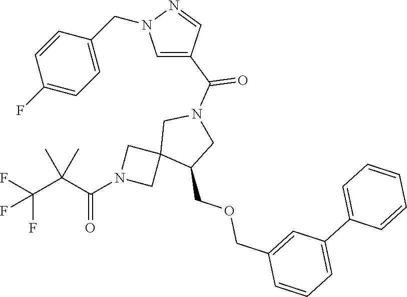 [SMILES: CC(C)(C(=O)N1CC2(CN(C(=O)c3cnn(Cc4ccc(F)cc4)c3)C[C@H]2COCc2cccc(-c3ccccc3)c2)C1)C(F)(F)F] |
| I′-40 | 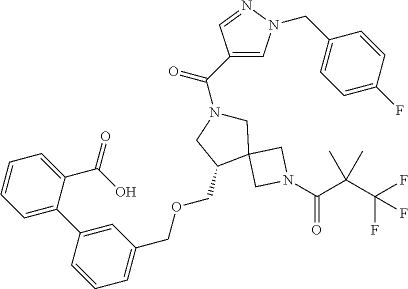 [SMILES: CC(C)(C(=O)N1CC2(CN(C(=O)c3cnn(Cc4ccc(F)cc4)c3)C[C@H]2COCc2cccc(-c3ccccc3C(=O)O)c2)C1)C(F)(F)F] |
| I′-41 | 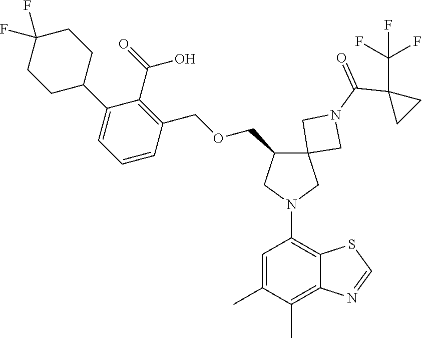 [SMILES: Cc1cc(N2C[C@@H](COCc3cccc(C4CCC(F)(F)CC4)c3C(=O)O)C3(CN(C(=O)C4(C(F)(F)F)CC4)C3)C2)c2scnc2c1C] |
| I′-42 | 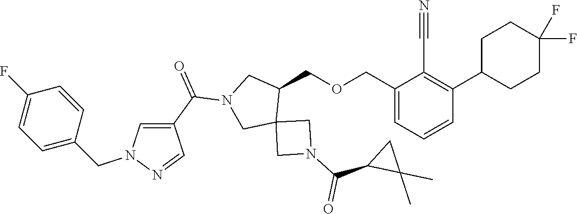 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C#N)C1] |
| I′-43 | 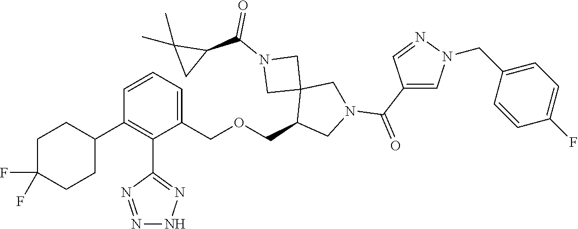 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2-c2nn[nH]n2)C1] |
| I′-44 | 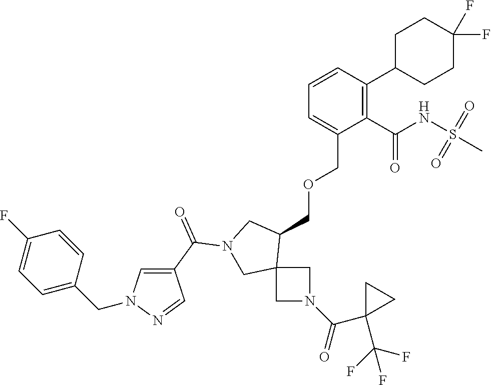 [SMILES: CS(=O)(=O)NC(=O)c1c(COC[C@@H]2CN(C(=O)c3cnn(Cc4ccc(F)cc4)c3)CC23CN(C(=O)C2(C(F)(F)F)CC2)C3)cccc1C1CCC(F)(F)CC1] |
| I′-45 | 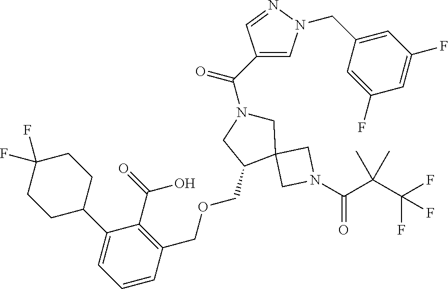 [SMILES: CC(C)(C(=O)N1CC2(CN(C(=O)c3cnn(Cc4cc(F)cc(F)c4)c3)C[C@H]2COCc2cccc(C3CCC(F)(F)CC3)c2C(=O)O)C1)C(F)(F)F] |
| I′-46 | 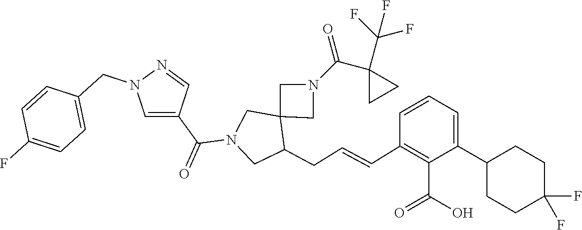 [SMILES: O=C(O)c1c(/C=C/CC2CN(C(=O)c3cnn(Cc4ccc(F)cc4)c3)CC23CN(C(=O)C2(C(F)(F)F)CC2)C3)cccc1C1CCC(F)(F)CC1] |
| I′-47 | 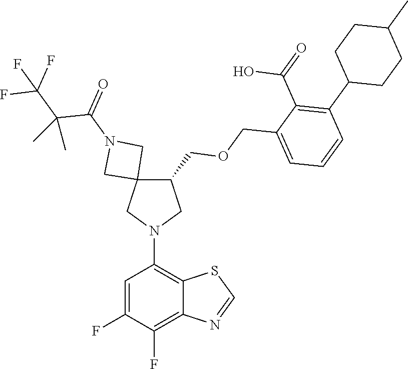 [SMILES: CC1CCC(c2cccc(COC[C@@H]3CN(c4cc(F)c(F)c5ncsc45)CC34CN(C(=O)C(C)(C)C(F)(F)F)C4)c2C(=O)O)CC1] |
| I′-48 | 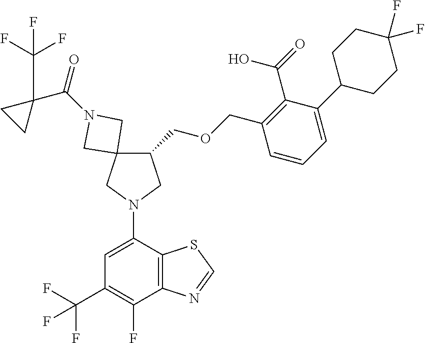 [SMILES: O=C(O)c1c(COC[C@@H]2CN(c3cc(C(F)(F)F)c(F)c4ncsc34)CC23CN(C(=O)C2(C(F)(F)F)CC2)C3)cccc1C1CCC(F)(F)CC1] |
| I′-49 | 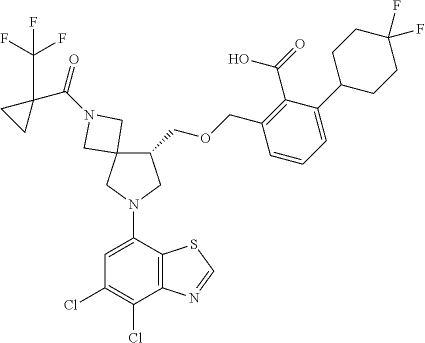 [SMILES: O=C(O)c1c(COC[C@@H]2CN(c3cc(Cl)c(Cl)c4ncsc34)CC23CN(C(=O)C2(C(F)(F)F)CC2)C3)cccc1C1CCC(F)(F)CC1] |
| I′-50 | 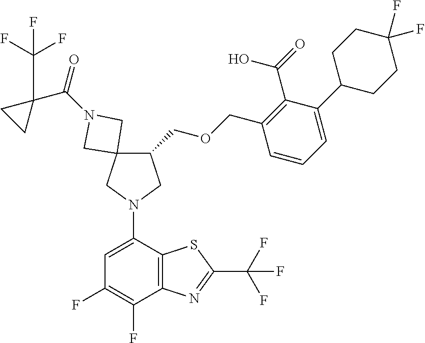 [SMILES: O=C(O)c1c(COC[C@@H]2CN(c3cc(F)c(F)c4nc(C(F)(F)F)sc34)CC23CN(C(=O)C2(C(F)(F)F)CC2)C3)cccc1C1CCC(F)(F)CC1] |
| I′-51 | 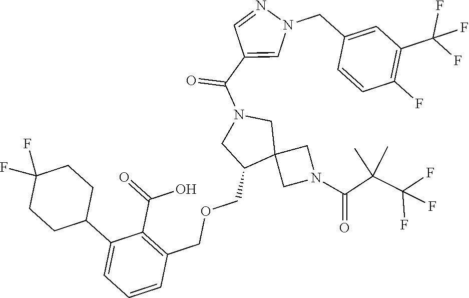 [SMILES: CC(C)(C(=O)N1CC2(CN(C(=O)c3cnn(Cc4ccc(F)c(C(F)(F)F)c4)c3)C[C@H]2COCc2cccc(C3CCC(F)(F)CC3)c2C(=O)O)C1)C(F)(F)F] |
| I′-52 | 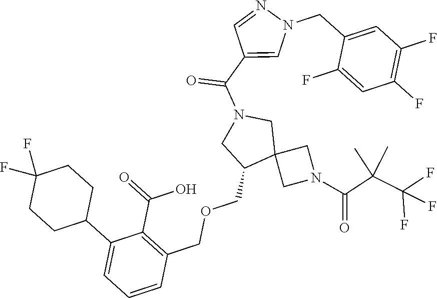 [SMILES: CC(C)(C(=O)N1CC2(CN(C(=O)c3cnn(Cc4cc(F)c(F)cc4F)c3)C[C@H]2COCc2cccc(C3CCC(F)(F)CC3)c2C(=O)O)C1)C(F)(F)F] |
| I′-53 | 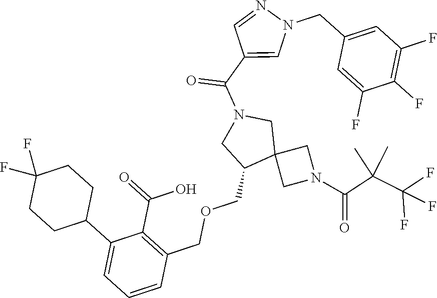 [SMILES: CC(C)(C(=O)N1CC2(CN(C(=O)c3cnn(Cc4cc(F)c(F)c(F)c4)c3)C[C@H]2COCc2cccc(C3CCC(F)(F)CC3)c2C(=O)O)C1)C(F)(F)F] |
| I′-54 | 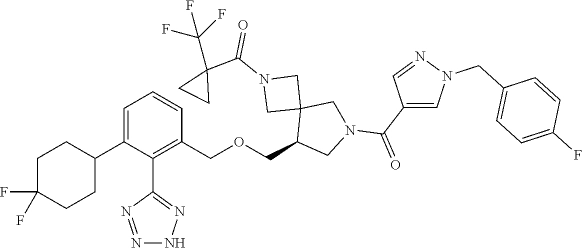 [SMILES: O=C(c1cnn(Cc2ccc(F)cc2)c1)N1C[C@@H](COCc2cccc(C3CCC(F)(F)CC3)c2-c2nn[nH]n2)C2(C1)CN(C(=O)C1(C(F)(F)F)CC1)C2] |
| I′-55 | 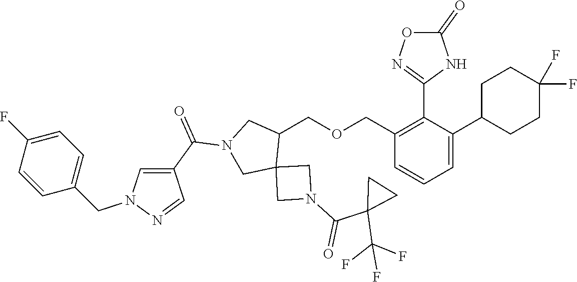 [SMILES: O=C(c1cnn(Cc2ccc(F)cc2)c1)N1CC(COCc2cccc(C3CCC(F)(F)CC3)c2-c2noc(=O)[nH]2)C2(C1)CN(C(=O)C1(C(F)(F)F)CC1)C2] |
| I′-56 | 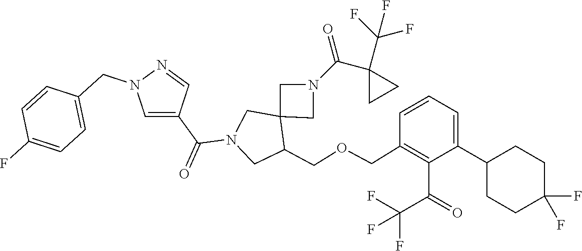 [SMILES: O=C(c1cnn(Cc2ccc(F)cc2)c1)N1CC(COCc2cccc(C3CCC(F)(F)CC3)c2C(=O)C(F)(F)F)C2(C1)CN(C(=O)C1(C(F)(F)F)CC1)C2] |
| I′-57 | 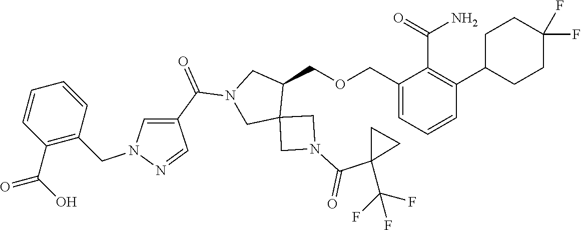 [SMILES: NC(=O)c1c(COC[C@@H]2CN(C(=O)c3cnn(Cc4ccccc4C(=O)O)c3)CC23CN(C(=O)C2(C(F)(F)F)CC2)C3)cccc1C1CCC(F)(F)CC1] |
| I′-58 | 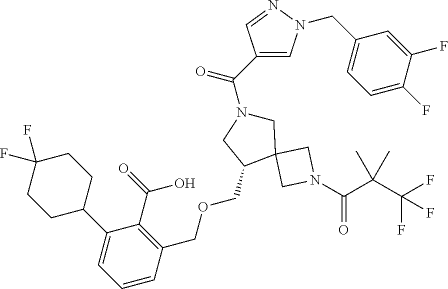 [SMILES: CC(C)(C(=O)N1CC2(CN(C(=O)c3cnn(Cc4ccc(F)c(F)c4)c3)C[C@H]2COCc2cccc(C3CCC(F)(F)CC3)c2C(=O)O)C1)C(F)(F)F] |
| I′-59 | 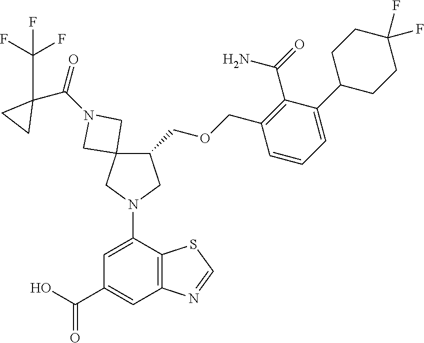 [SMILES: NC(=O)c1c(COC[C@@H]2CN(c3cc(C(=O)O)cc4ncsc34)CC23CN(C(=O)C2(C(F)(F)F)CC2)C3)cccc1C1CCC(F)(F)CC1] |
| I′-60 | 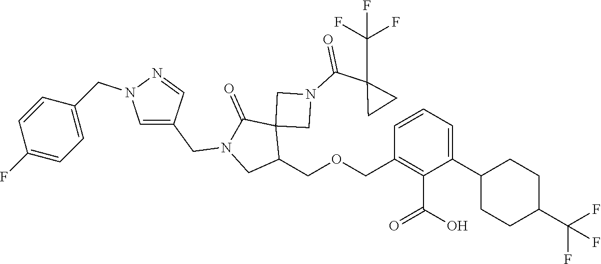 [SMILES: O=C(O)c1c(COCC2CN(Cc3cnn(Cc4ccc(F)cc4)c3)C(=O)C23CN(C(=O)C2(C(F)(F)F)CC2)C3)cccc1C1CCC(C(F)(F)F)CC1] |
| I′-61 | 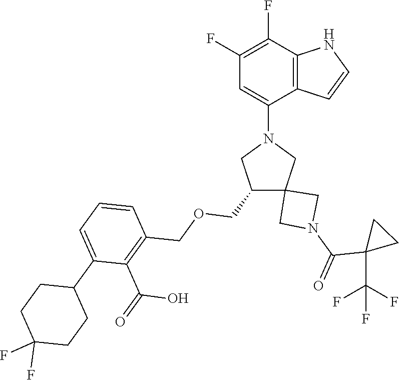 [SMILES: O=C(O)c1c(COC[C@@H]2CN(c3cc(F)c(F)c4[nH]ccc34)CC23CN(C(=O)C2(C(F)(F)F)CC2)C3)cccc1C1CCC(F)(F)CC1] |
| I′-62 | 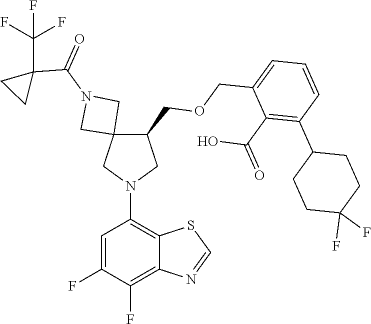 [SMILES: O=C(O)c1c(COC[C@H]2CN(c3cc(F)c(F)c4ncsc34)CC23CN(C(=O)C2(C(F)(F)F)CC2)C3)cccc1C1CCC(F)(F)CC1] |
| I′-63 | 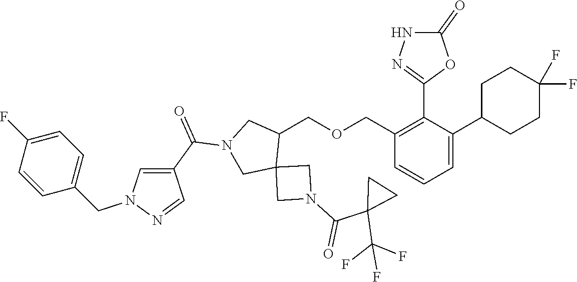 [SMILES: O=C(c1cnn(Cc2ccc(F)cc2)c1)N1CC(COCc2cccc(C3CCC(F)(F)CC3)c2-c2n[nH]c(=O)o2)C2(C1)CN(C(=O)C1(C(F)(F)F)CC1)C2] |
| I′-64 | 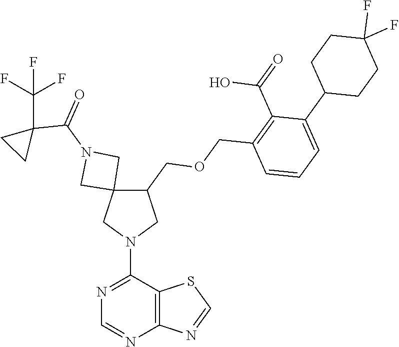 [SMILES: O=C(O)c1c(COCC2CN(c3ncnc4ncsc34)CC23CN(C(=O)C2(C(F)(F)F)CC2)C3)cccc1C1CCC(F)(F)CC1] |
| I′-65 | 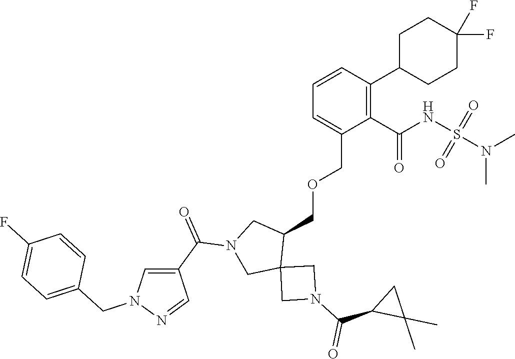 [SMILES: CN(C)S(=O)(=O)NC(=O)c1c(COC[C@@H]2CN(C(=O)c3cnn(Cc4ccc(F)cc4)c3)CC23CN(C(=O)[C@H]2CC2(C)C)C3)cccc1C1CCC(F)(F)CC1] |
| I′-66 | 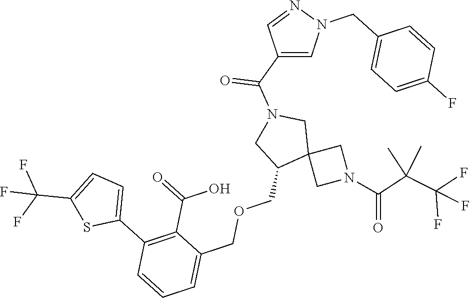 [SMILES: CC(C)(C(=O)N1CC2(CN(C(=O)c3cnn(Cc4ccc(F)cc4)c3)C[C@H]2COCc2cccc(-c3ccc(C(F)(F)F)s3)c2C(=O)O)C1)C(F)(F)F] |
| I′-67 | 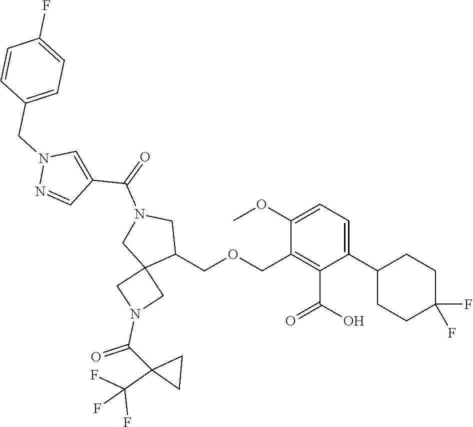 [SMILES: COc1ccc(C2CCC(F)(F)CC2)c(C(=O)O)c1COCC1CN(C(=O)c2cnn(Cc3ccc(F)cc3)c2)CC12CN(C(=O)C1(C(F)(F)F)CC1)C2] |
| I′-68 | 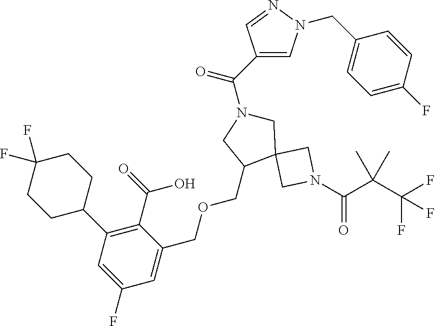 [SMILES: CC(C)(C(=O)N1CC2(CN(C(=O)c3cnn(Cc4ccc(F)cc4)c3)CC2COCc2cc(F)cc(C3CCC(F)(F)CC3)c2C(=O)O)C1)C(F)(F)F] |
| I′-69 | 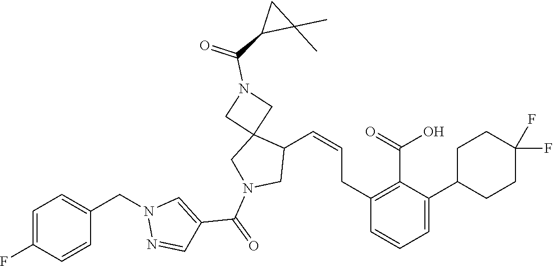 [SMILES: CC1(C)C[C@@H]1C(=O)N1CC2(CN(C(=O)c3cnn(Cc4ccc(F)cc4)c3)CC2/C=C\Cc2cccc(C3CCC(F)(F)CC3)c2C(=O)O)C1] |
| I′-70 | 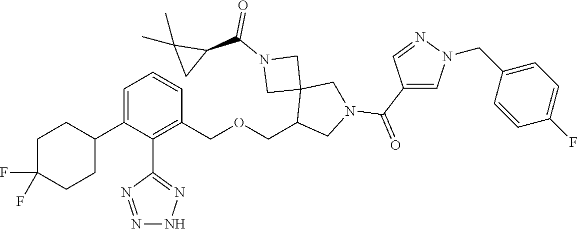 [SMILES: CC1(C)C[C@@H]1C(=O)N1CC2(CN(C(=O)c3cnn(Cc4ccc(F)cc4)c3)CC2COCc2cccc(C3CCC(F)(F)CC3)c2-c2nn[nH]n2)C1] |
| I′-71 | 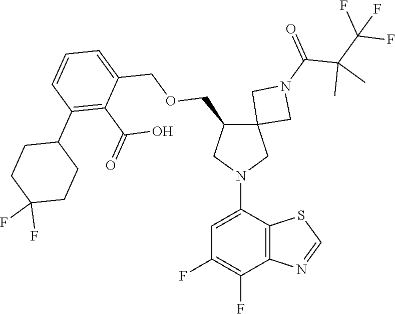 [SMILES: CC(C)(C(=O)N1CC2(C1)CN(c1cc(F)c(F)c3ncsc13)C[C@H]2COCc1cccc(C2CCC(F)(F)CC2)c1C(=O)O)C(F)(F)F] |
| I′-72 | 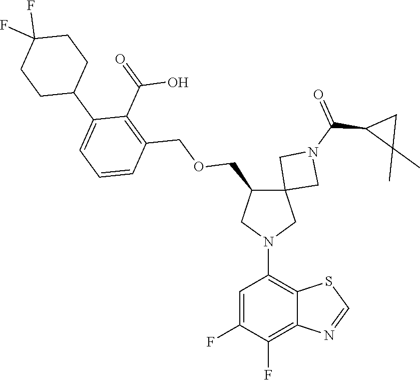 [SMILES: CC1(C)C[C@@H]1C(=O)N1CC2(C1)CN(c1cc(F)c(F)c3ncsc13)C[C@H]2COCc1cccc(C2CCC(F)(F)CC2)c1C(=O)O] |
| I′-73 | 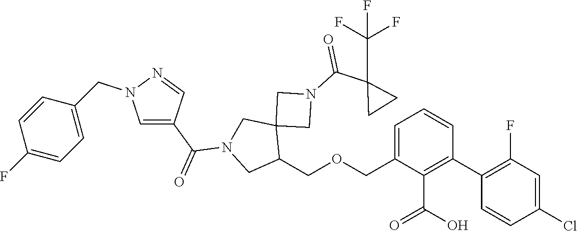 [SMILES: O=C(O)c1c(COCC2CN(C(=O)c3cnn(Cc4ccc(F)cc4)c3)CC23CN(C(=O)C2(C(F)(F)F)CC2)C3)cccc1-c1ccc(Cl)cc1F] |
| I′-74 | 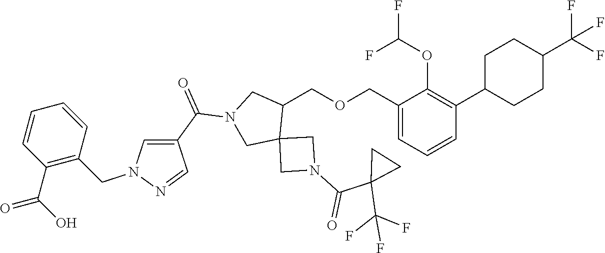 [SMILES: O=C(O)c1ccccc1Cn1cc(C(=O)N2CC(COCc3cccc(C4CCC(C(F)(F)F)CC4)c3OC(F)F)C3(C2)CN(C(=O)C2(C(F)(F)F)CC2)C3)cn1] |
| I′-75 | 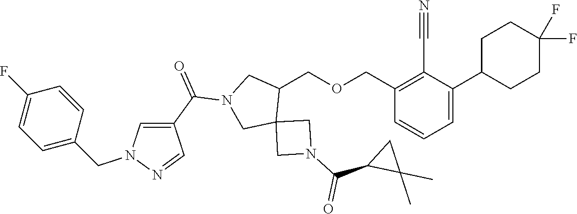 [SMILES: CC1(C)C[C@@H]1C(=O)N1CC2(CN(C(=O)c3cnn(Cc4ccc(F)cc4)c3)CC2COCc2cccc(C3CCC(F)(F)CC3)c2C#N)C1] |
| I′-76 | 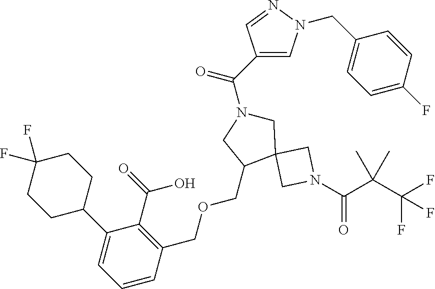 [SMILES: CC(C)(C(=O)N1CC2(CN(C(=O)c3cnn(Cc4ccc(F)cc4)c3)CC2COCc2cccc(C3CCC(F)(F)CC3)c2C(=O)O)C1)C(F)(F)F] |
| I′-77 | 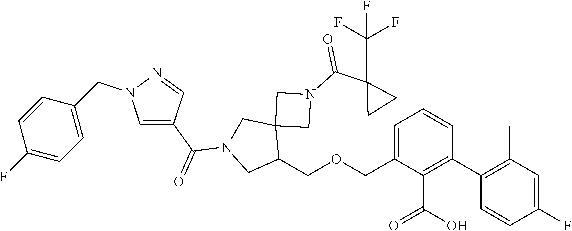 [SMILES: Cc1cc(F)ccc1-c1cccc(COCC2CN(C(=O)c3cnn(Cc4ccc(F)cc4)c3)CC23CN(C(=O)C2(C(F)(F)F)CC2)C3)c1C(=O)O] |
| I′-78 | 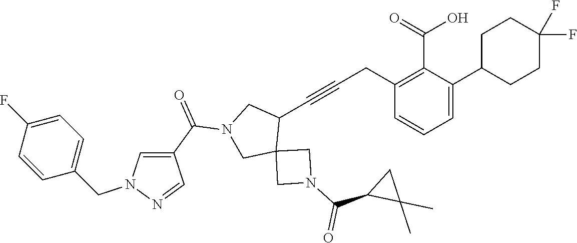 [SMILES: CC1(C)C[C@@H]1C(=O)N1CC2(CN(C(=O)c3cnn(Cc4ccc(F)cc4)c3)CC2C#CCc2cccc(C3CCC(F)(F)CC3)c2C(=O)O)C1] |
| I′-79 | 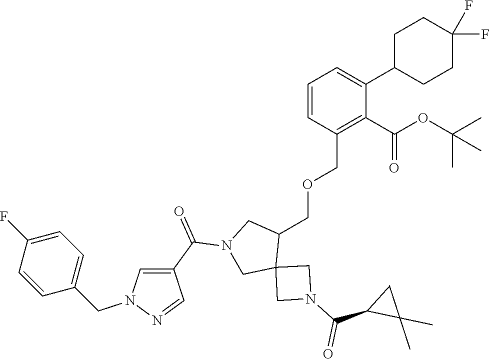 [SMILES: CC(C)(C)OC(=O)c1c(COCC2CN(C(=O)c3cnn(Cc4ccc(F)cc4)c3)CC23CN(C(=O)[C@H]2CC2(C)C)C3)cccc1C1CCC(F)(F)CC1] |
| I′-80 | 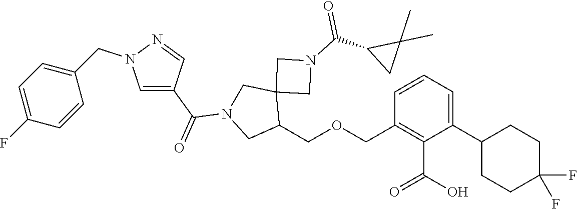 [SMILES: CC1(C)C[C@@H]1C(=O)N1CC2(CN(C(=O)c3cnn(Cc4ccc(F)cc4)c3)CC2COCc2cccc(C3CCC(F)(F)CC3)c2C(=O)O)C1] |
| I′-81 | 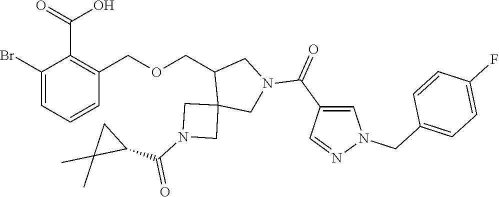 [SMILES: CC1(C)C[C@@H]1C(=O)N1CC2(CN(C(=O)c3cnn(Cc4ccc(F)cc4)c3)CC2COCc2cccc(Br)c2C(=O)O)C1] |
| I′-82 | 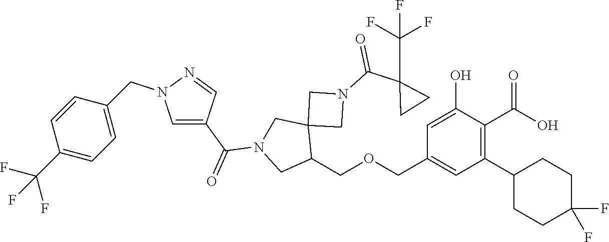 [SMILES: O=C(O)c1c(O)cc(COCC2CN(C(=O)c3cnn(Cc4ccc(C(F)(F)F)cc4)c3)CC23CN(C(=O)C2(C(F)(F)F)CC2)C3)cc1C1CCC(F)(F)CC1] |
| I′-83 | 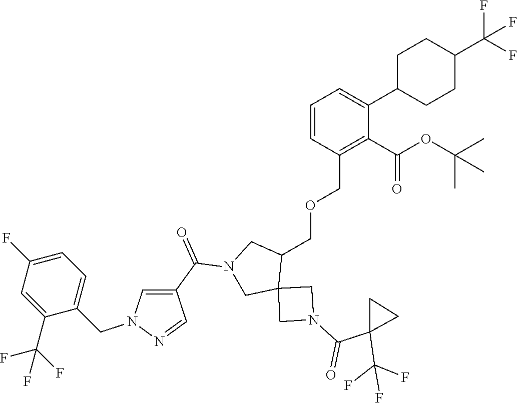 [SMILES: CC(C)(C)OC(=O)c1c(COCC2CN(C(=O)c3cnn(Cc4ccc(F)cc4C(F)(F)F)c3)CC23CN(C(=O)C2(C(F)(F)F)CC2)C3)cccc1C1CCC(C(F)(F)F)CC1] |
| I′-84 | 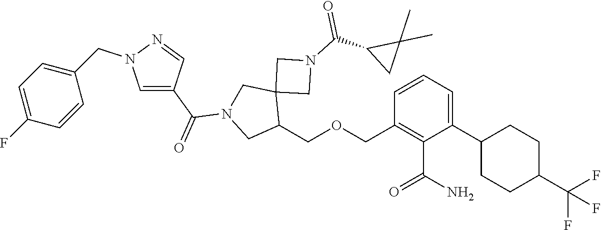 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(N)=O)C1] |
| I′-85 | 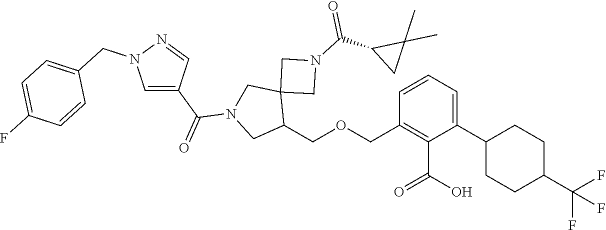 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(=O)O)C1] |
| I′-86 | 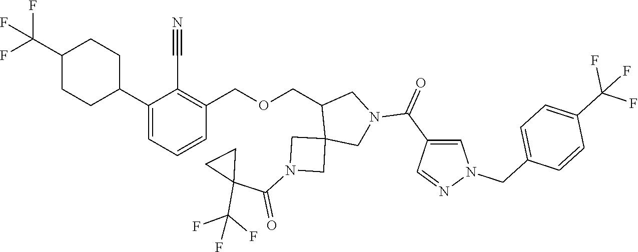 [SMILES: N#Cc1c(COCC2CN(C(=O)c3cnn(Cc4ccc(C(F)(F)F)cc4)c3)CC23CN(C(=O)C2(C(F)(F)F)CC2)C3)cccc1C1CCC(C(F)(F)F)CC1] |
| I′-87 | 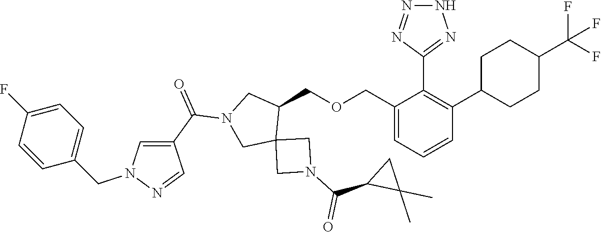 [SMILES: CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2-c2nn[nH]n2)C1] |
| I′-88 | 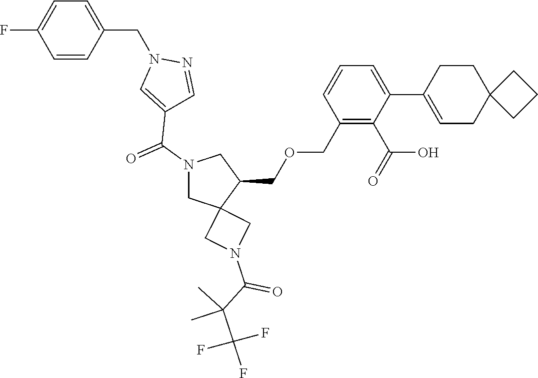 [SMILES: CC(C)(C(=O)N1CC2(CN(C(=O)c3cnn(Cc4ccc(F)cc4)c3)C[C@H]2COCc2cccc(C3=CCC4(CCC4)CC3)c2C(=O)O)C1)C(F)(F)F] |
| I′-89 | 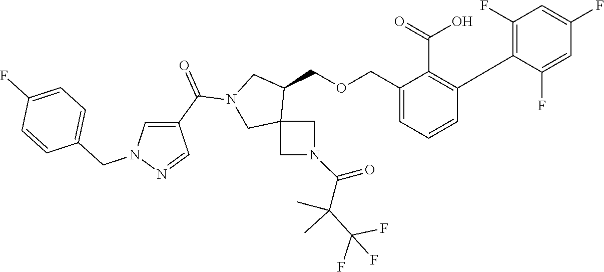 |
| I′-90 | 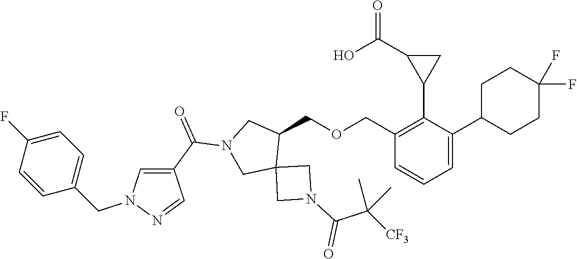 [SMILES: CC(C)(C(=O)N1CC2(CN(C(=O)c3cnn(Cc4ccc(F)cc4)c3)C[C@H]2COCc2cccc(C3CCC(F)(F)CC3)c2C2CC2C(=O)O)C1)C(F)(F)F] |
| I′-91 | 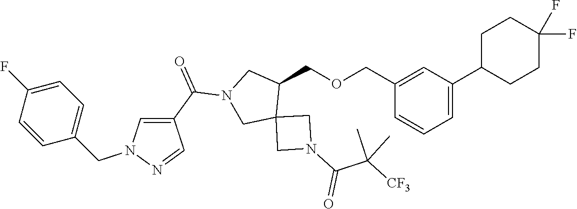 [SMILES: CC(C)(C(=O)N1CC2(CN(C(=O)c3cnn(Cc4ccc(F)cc4)c3)C[C@H]2COCc2cccc(C3CCC(F)(F)CC3)c2)C1)C(F)(F)F] |
| I′-92 | 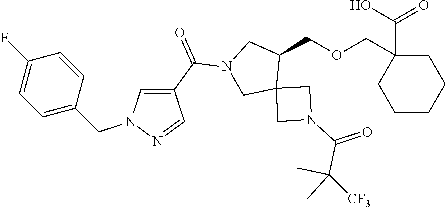 [SMILES: CC(C)(C(=O)N1CC2(CN(C(=O)c3cnn(Cc4ccc(F)cc4)c3)C[C@H]2COCC2(C(=O)O)CCCCC2)C1)C(F)(F)F] |
| I′-93 | 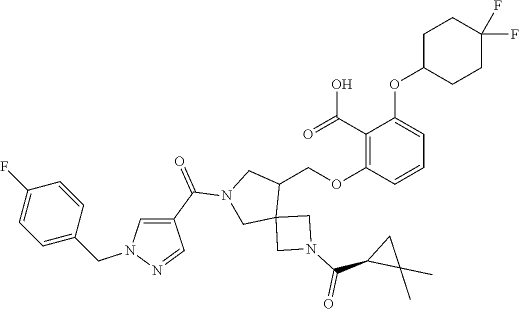 [SMILES: CC1(C)C[C@@H]1C(=O)N1CC2(CN(C(=O)c3cnn(Cc4ccc(F)cc4)c3)CC2COc2cccc(OC3CCC(F)(F)CC3)c2C(=O)O)C1] |
| I′-94 | 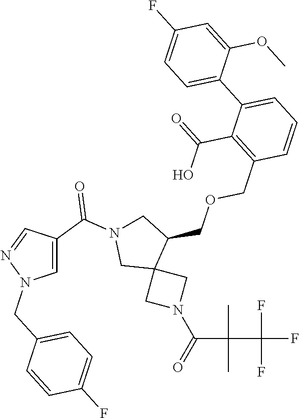 [SMILES: COc1cc(F)ccc1-c1cccc(COC[C@@H]2CN(C(=O)c3cnn(Cc4ccc(F)cc4)c3)CC23CN(C(=O)C(C)(C)C(F)(F)F)C3)c1C(=O)O] |
| I′-95 | 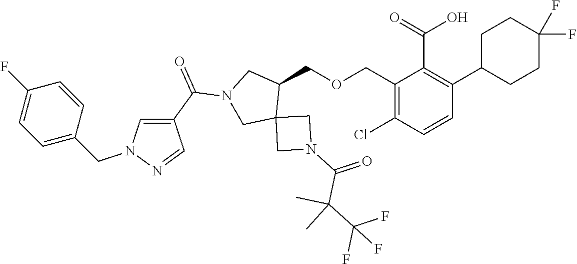 [SMILES: CC(C)(C(=O)N1CC2(CN(C(=O)c3cnn(Cc4ccc(F)cc4)c3)C[C@H]2COCc2c(Cl)ccc(C3CCC(F)(F)CC3)c2C(=O)O)C1)C(F)(F)F] |
| I′-96 | 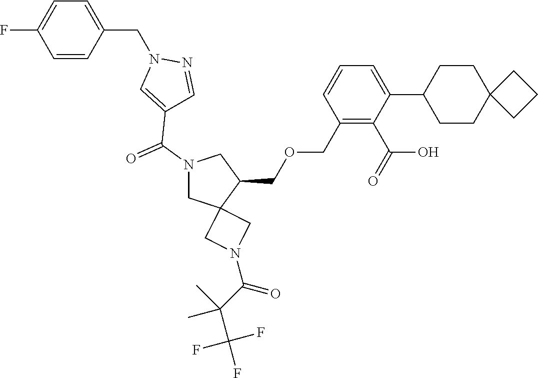 [SMILES: CC(C)(C(=O)N1CC2(CN(C(=O)c3cnn(Cc4ccc(F)cc4)c3)C[C@H]2COCc2cccc(C3CCC4(CCC4)CC3)c2C(=O)O)C1)C(F)(F)F] |
| I′-97 | 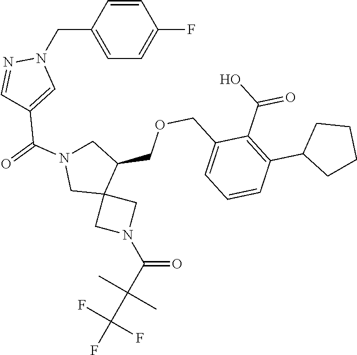 [SMILES: CC(C)(C(=O)N1CC2(CN(C(=O)c3cnn(Cc4ccc(F)cc4)c3)C[C@H]2COCc2cccc(C3CCCC3)c2C(=O)O)C1)C(F)(F)F] |
| I′-98 | 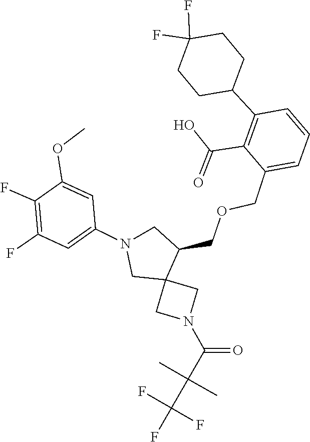 [SMILES: COc1cc(N2C[C@@H](COCc3cccc(C4CCC(F)(F)CC4)c3C(=O)O)C3(CN(C(=O)C(C)(C)C(F)(F)F)C3)C2)cc(F)c1F] |
| I′-99 | 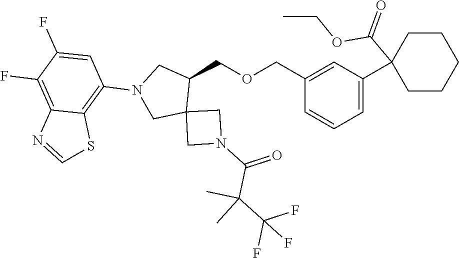 [SMILES: CCOC(=O)C1(c2cccc(COC[C@@H]3CN(c4cc(F)c(F)c5ncsc45)CC34CN(C(=O)C(C)(C)C(F)(F)F)C4)c2)CCCCC1] |
| I′-100 | 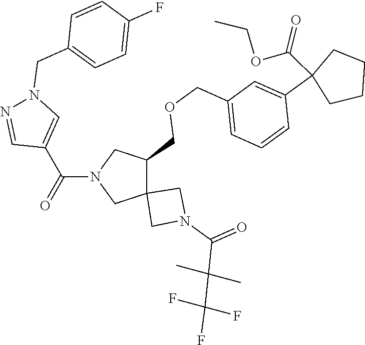 [SMILES: CCOC(=O)C1(c2cccc(COC[C@@H]3CN(C(=O)c4cnn(Cc5ccc(F)cc5)c4)CC34CN(C(=O)C(C)(C)C(F)(F)F)C4)c2)CCCC1] |
| I′-101 | 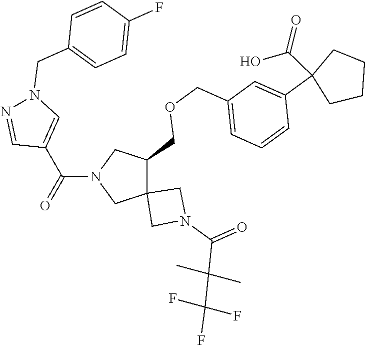 [SMILES: CC(C)(C(=O)N1CC2(CN(C(=O)c3cnn(Cc4ccc(F)cc4)c3)C[C@H]2COCc2cccc(C3(C(=O)O)CCCC3)c2)C1)C(F)(F)F] |
| I′-102 | 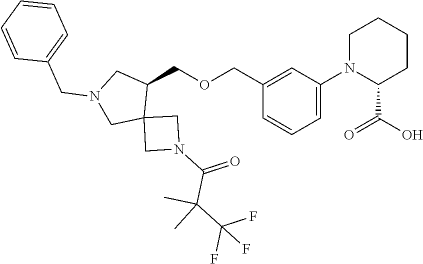 [SMILES: CC(C)(C(=O)N1CC2(CN(Cc3ccccc3)C[C@H]2COCc2cccc(N3CCCC[C@@H]3C(=O)O)c2)C1)C(F)(F)F] |
| I′-103 | 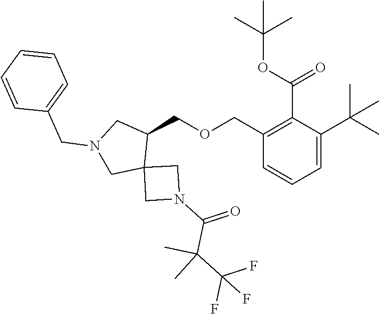 [SMILES: CC(C)(C)OC(=O)c1c(COC[C@@H]2CN(Cc3ccccc3)CC23CN(C(=O)C(C)(C)C(F)(F)F)C3)cccc1C(C)(C)C] |
| I′-104 | 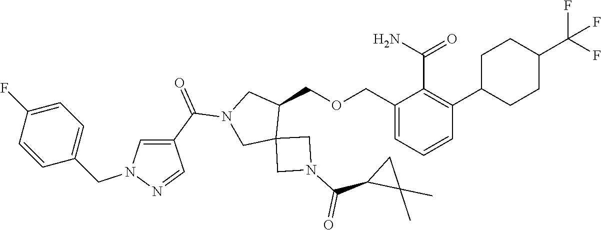 [SMILES: CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2C(N)=O)C1] |

The present disclosure contemplates any and all enantiomers, diastereomers and conformation isomers of a compound shown herein.

In some embodiments, the present disclosure provides a compound set forth in Table 8A, above, or a pharmaceutically acceptable salt thereof. In some embodiments, the disclosure provides a compound set forth in Table 8A, above, or a pharmaceutically acceptable salt thereof, and any enantiomers, diastereomers, or conformation isomers thereof.

In some embodiments, the present disclosure provides a compound set forth in Table 8B, above, or a pharmaceutically acceptable salt thereof. In some embodiments, the disclosure provides a compound set forth in Table 8B, above, or a pharmaceutically acceptable salt thereof, and any enantiomers, diastereomers, or conformation isomers thereof.

In some embodiments, the present disclosure provides a pharmaceutical composition comprising a compound of the present disclosure, or a pharmaceutically acceptable salt thereof, together with a pharmaceutically acceptable carrier, excipient, vehicle, adjuvant or diluent. In some embodiments, the present disclosure provides a pharmaceutical composition comprising a compound set forth in Table 8A or Table 8B above, or a pharmaceutically acceptable salt thereof, together with a pharmaceutically acceptable carrier, excipient, vehicle, adjuvant or diluent. In some embodiments, the pharmaceutical composition further comprises an additional therapeutic agent.

In some embodiments, the present disclosure provides a complex comprising a CDK2 protein and a compound of the present disclosure.

In some embodiments, the present disclosure provides a method of inhibiting the activity of a cyclin-dependent kinase (CDK). In some embodiments, the method comprises contacting a compound of the present disclosure with a CDK. In some embodiments, the compound and the CDK are contacted in vivo. In some embodiments, the compound and the CDK are contacted in vitro. In some embodiments, the CDK is selected from CDK1, CDK2, CDK3, CDK4, CDK5, CDK6, CDK7, CDK8, CDK9, CDK10, CDK11, CDK12 and CDK13. In some embodiments, the CDK is CDK2. In some embodiments, the CDK is CDK3. In some embodiments, the CDK is CDK4. In some embodiments, the CDK is CDK6. In some embodiments, the method inhibits the activity of both CDK2 and CDK3. In some embodiments, the method inhibits the activity of CDK2 and one or both of CDK4 and CDK6.

In some embodiments, the compounds of the present disclosure inhibit the activity of one or more CDKs selected from CDK1, CDK2, CDK3, CDK4, CDK5, CDK6, CDK7, CDK8, CDK9, CDK10, CDK11, CDK12 and CDK13. In some embodiments, the compounds of the present disclosure inhibit CDK2. In some embodiments, the compounds of the present disclosure inhibit CDK3. In some embodiments, the compounds of the present disclosure inhibit CDK4. In some embodiments, the compounds of the present disclosure inhibit CDK5. In some embodiments, the compounds of the present disclosure inhibit CDK6. In some embodiments, the compounds of the present disclosure are CDK2/3 inhibitors. In some embodiments, the compounds of the present disclosure are CDK2/4/6 inhibitors.

In some embodiments, the present disclosure provides compounds that selectively inhibit CDK2 over other cyclin-dependent kinases (CDKs). In some embodiments, the compounds of the present disclosure selectively inhibit CDK2 over one or more other CDKs, selected from CDK1, CDK3, CDK4, CDK5, CDK6, CDK7, CDK8, CDK9, CDK10, CDK11, CDK12 and CDK13. In some embodiments, the compounds of the present disclosure selectively inhibit CDK2 over CDK4. In some embodiments, the compounds of the present disclosure selectively inhibit CDK2 over CDK6. In some embodiments, the compounds of the present disclosure selectively inhibit CDK2 over CDK4 and CDK6.

In some embodiments, the present disclosure provides compounds that selectively inhibit CDK2/cyclin E complexes over other CDK complexes.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

Compounds of the present disclosure, including those of Formula I or Formula I′ and the compounds of Table 8A or Table 8B, can generally be prepared according the schemes and methods described below. Reagents and conditions can be modified and substituted using knowledge common to one of ordinary skill in the art, as needed, in order to arrive at the compounds of the present disclosure.

Spriocyclic precursors (i.e., compounds G-I) to compounds of the disclosure, may be prepared according to Scheme 1 and subsequently modified with protecting groups to furnish J of Scheme 2, then functionalized at the spirocyclic nitrogens to include the side groups of M, reduced to an alcohol N, and functionalized via nucleophilic substitution (Sn2) chemistry to furnish compounds of the disclosure P. To prepare the spirocycles, Horner-Wadsworth-Emmons reaction between A and B furnishes C which when reacted with tertiary amine D produces the spirocyclic core of the present compounds in compound E. E may be further functionalized, deprotected and/or protected using orthogonal protecting group strategies as known in the art to protect or deprotect either of the spirocyclic amines or pendant carboxylic acid to furnish the appropriate spirocycle for completing the compounds, for example, compounds G-I. Alternatively, where an amine L⁴ linker is desired as in S, reductive amination chemistry as in Scheme 3 may be employed to complete the R^{A} moiety.

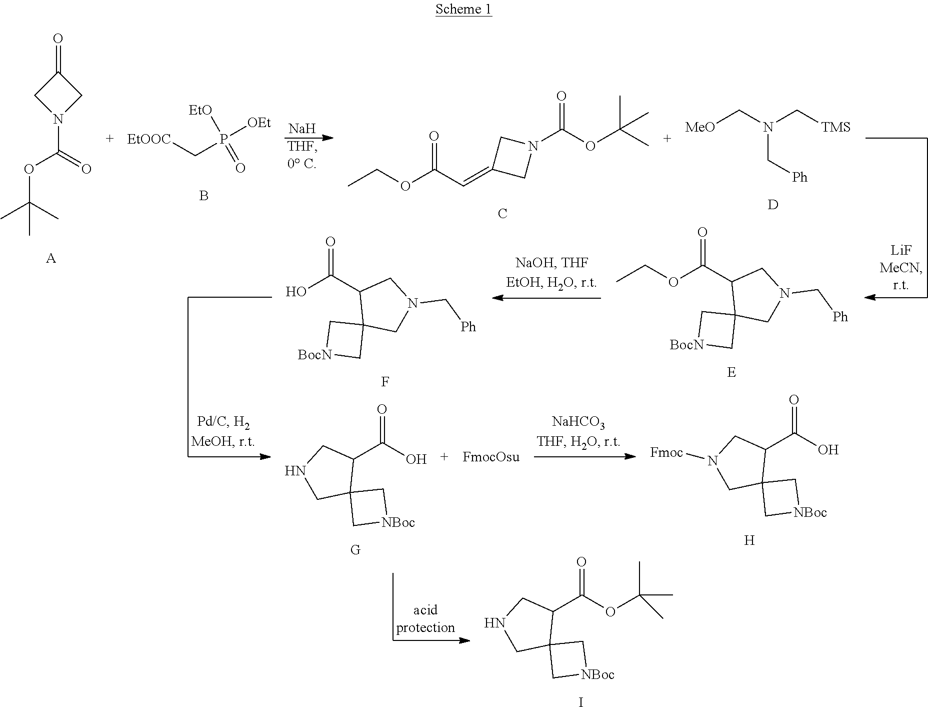

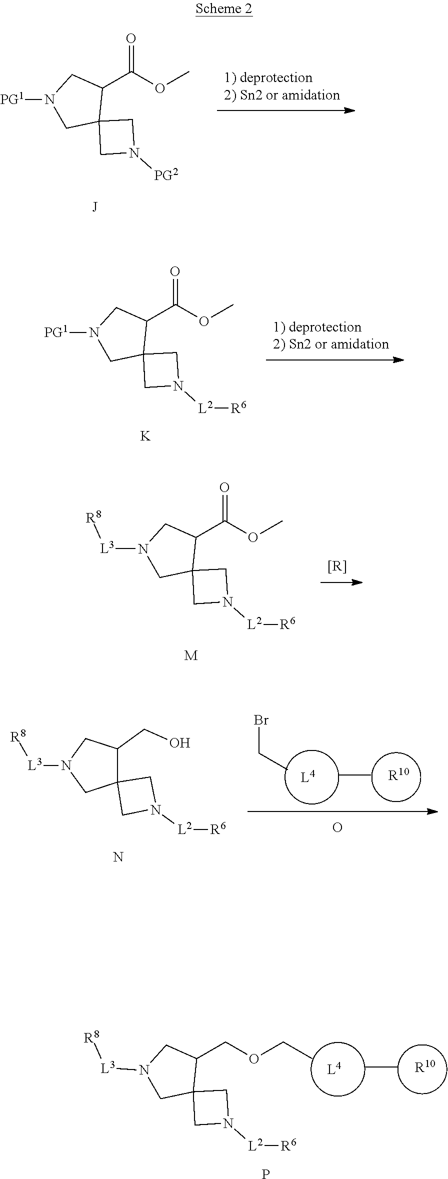

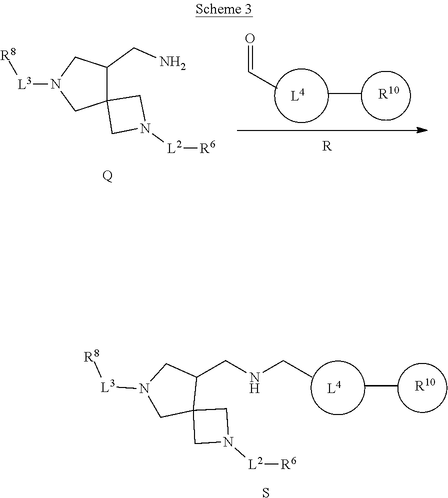

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably inhibit a CDK2 protein, or a mutant thereof, in a biological sample or in a patient. In certain embodiments, the amount of compound in compositions of this disclosure is such that it is effective to measurably inhibit a CDK2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered subcutaneously, orally, intraperitoneally or intravenously. In some embodiments, the compositions are administered orally. In some embodiments, the compositions are administered intraperitoneally. In some embodiments, the compositions are administered intravenously. In some embodiments, the compositions are administered subcutaneously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the modulation of the activity CDK2. In some embodiments, the compounds and compositions described herein are CDK2 inhibitors.

In some embodiments, the compounds and compositions of the present disclosure are useful for treating diseases and disorders associated with CDK2 activity, including, but not limited to cancers, myeloproliferative disorders, autoimmune disorders, inflammatory disorders, viral infections, fibrotic disorders, and neurodegenerative disorders.

In some embodiments, the disclosure provides a method of inhibiting the activity of a CDK2, the method comprising contacting a compound of the present disclosure, or a pharmaceutically acceptable salt thereof with the CDK2. In some embodiments, the contacting takes place in vitro. In some embodiments, the contacting takes place in vivo.

In some embodiments, the disclosure provides a method of treating, preventing or lessening the severity of a disease or disorder associated with CDK2 activity in a patient, including, but not limited to cancers, myeloproliferative disorders, autoimmune disorders, inflammatory disorders, fibrotic disorders, and neurodegenerative disorders, said method comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the treatment of a disease or disorder associated with CDK2 activity.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the manufacture of a medicament for treating a disease or disorder associated with CDK2 activity.

In some embodiments, the disease or disorder associated with CDK2 activity is a CDK2-mediated disease or disorder. In some embodiments, the disease or disorder associated with CDK2 activity is a disease or disorder caused by CDK2 over-activity.

In some embodiments, the disease or disorder associated with CDK2 activity is cancer.

In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

In some embodiments, the cancer is characterized by amplification or overexpression of CCNE1 and/or CCNE2.

In some embodiments, the cancer is breast cancer. In some embodiments, the breast cancer is a breast cancer selected from ER-positive/HR-positive breast cancer, HER2-negative breast cancer, ER-positive/HR-positive breast cancer, HER2-positive breast cancer, triple negative breast cancer (TNBC), inflammatory breast cancer, endocrine resistant breast cancer, trastuzumab resistant breast cancer, breast cancer with primary or acquired resistance to CDK4/CDK6 inhibition, advanced breast cancer and metastatic breast cancer. In some embodiments the breast cancer is characterized by amplification or overexpression of CCNE1 and/or CCNE2.

In some embodiments, the cancer is ovarian cancer. In some embodiments, the ovarian cancer is high-grade serous ovarian cancer (HGSOC). In some embodiments the ovarian cancer is characterized by amplification or overexpression of CCNE1 and/or CCNE2.

In some embodiments, the cancer is bladder cancer. In some embodiments, the bladder cancer is characterized by amplification or overexpression of CCNE1 and/or CCNE2.

In some embodiments, the cancer is uterine cancer. In some embodiments, the uterine cancer is characterized by amplification or overexpression of CCNE1 and/or CCNE2.

In some embodiments, the cancer is prostate cancer. In some embodiments, the prostate cancer is characterized by amplification or overexpression of CCNE1 and/or CCNE2.

In some embodiments, the cancer is lung cancer. In some embodiments, the lung cancer is a lung cancer selected from non-small cell lung cancer, small cell lung cancer, squamous cell carcinoma, adenocarcinoma, and mesothelioma. In some embodiments, the lung cancer is characterized by amplification or overexpression of CCNE1 and/or CCNE2. In some embodiments, the lung cancer is CCNE1 amplified squamous cell carcinoma or CCNE1 amplified adenocarcinoma.

In some embodiments, the cancer is head and neck cancer. In some embodiments, the head and neck cancer is characterized by amplification or overexpression of CCNE1 and/or CCNE2.

In some embodiments, the cancer is colorectal cancer. In some embodiments, the colorectal cancer is characterized by amplification or overexpression of CCNE1 and/or CCNE2.

In some embodiments, the cancer is kidney cancer. In some embodiments, the kidney cancer is renal cell carcinoma (RCC). In some embodiments, the kidney cancer is characterized by amplification or overexpression of CCNE1 and/or CCNE2.

In some embodiments, the cancer is liver cancer. In some embodiments, the liver cancer is hepatocellular carcinoma (HCC). In some embodiments, the liver cancer is characterized by amplification or overexpression of CCNE1 and/or CCNE2.

In some embodiments, the cancer is pancreatic cancer. In some embodiments, the pancreatic cancer is characterized by amplification or overexpression of CCNE1 and/or CCNE2.

In some embodiments, the cancer is stomach cancer. In some embodiments, the stomach cancer is characterized by amplification or overexpression of CCNE1 and/or CCNE2.

In some embodiments, the cancer is melanoma. In some embodiments, the melanoma is characterized by amplification or overexpression of CCNE1 and/or CCNE2. CDK2 expression is regulated by essential melanocytic transcription factor MITF. It has been found that CDK2 depletion suppresses the growth of melanoma (Du et al., Cancer Cell. 2004 December; 6(6): 565-576)

In some embodiments, the cancer is thyroid cancer. In some embodiments, the thyroid cancer is characterized by amplification or overexpression of CCNE1 and/or CCNE2.

In some embodiments, the disease or disorder associated with CDK2 activity is a myeloproliferative disorder.

In some embodiments, the disease or disorder associated with CDK2 activity is a neurodegenerative disease or disorder. In some embodiments, the neurodegenerative disease or disorder is Alzheimer's disease (AD). It has been reported that neuronal cell death in subjects suffering from AD is preceded by cell cycle events. Inhibition of one or more CDKs can inhibit cell cycle events and therefore stave off neuronal cell death (Yang et al., J Neurosci. 2003 Apr. 1; 23(7):2557-2563).

In some embodiments, the disease or disorder associated with CDK2 activity is a liver disease.

In some embodiments, the disease or disorder associated with CDK2 activity is liver fibrosis. It has been reported that CCNE1 knockout mice do not develop liver fibrosis upon exposure to pro-fibrotic toxin CCl₄, suggesting that liver fibrosis can be treated via administration of a CDK2 inhibitor (Nevzorova, et al., Hepatology. 2012 September; 56(3): 1140-1149).

In some embodiments, the disease or disorder associated with CDK2 activity is Cushing disease. Pituitary cyclin E/E2F1 signaling is a molecular mechanism underlying neuroendocrine regulation of the hypothalamic-pituitary-adrenal axis, and therefore provides a subcellular therapeutic target for CDK2 inhibitors of pituitary ACTH-dependent hypercortisolism, also known as Cushing disease (Liu, et al., J Clin Endocrinol Metab. 2015 July; 100(7): 2557-2564).

In some embodiments, the disease or disorder associated with CDK2 activity is a kidney disease.

In some embodiments, the disease or disorder associated with CDK2 activity is polycystic kidney disease. It has been reported that CDK2/CDK5 inhibitor roscovitine yields effective arrest of cystic kidney disease in mouse models of polycystic kidney disease (Bukanov, et al., Nature. 2006 Dec. 14; 444(7121):949-52).

In some embodiments, the disease or disorder associated with CDK2 activity is an autoimmune disorder. CDK2 ablation has been shown to promote immune tolerance by supporting the function of regulatory T cells (Chunder et al., J Immunol. 2012 Dec. 15; 189(12):5659-66).

In some embodiments, the disease or disorder associated with CDK2 activity is an inflammatory disorder. Cyclin E ablation has been shown to attenuate hepatitis in mice, while p27 knockout mice display exacerbation of renal inflammation (Ehedego et al., Oncogene. 2018 June; 37(25):3329-3339.; Ophascharoensuk et al., Nat Med. 1998 May; 4(5):575-80). In some embodiments, the inflammatory disorder is hepatitis.

In some embodiments, the compounds and compositions of the present disclosure are useful as male contraceptives. Based on the finding that male CDK2 knockout mice are sterile, CDK2 inhibitors have been studied as possible male contraceptives (Faber, et al., Biol Reprod. 2020 August; 103(2): 357-367). In some embodiments, the present disclosure provides a method of reducing male fertility comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

In some embodiments, the compounds and compositions of the present disclosure are useful for treating diseases and disorders associated with CDK5 activity, including, but not limited to cancers, myeloproliferative disorders, autoimmune disorders, inflammatory disorders, viral infections, fibrotic disorders, and neurodegenerative disorders. In some embodiments, the compounds and compositions of the present disclosure are useful for treating neurodegenerative disorders associated with CDK5 activity.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents that the compounds of the present disclosure may also be combined with include, without limitation: endocrine therapeutic agents, chemotherapeutic agents and other CDK inhibitory compounds.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of an endocrine therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional CDK inhibitory compounds. In some embodiments, the one or more additional CDK inhibitory compounds are CDK4, or CDK4/CDK6 inhibitors. In some embodiments, the one or more additional CDK inhibitory compounds are CDK4, CDK6, CDK7 or CDK4/CDK6 inhibitors. In some embodiments, the one or more additional CDK inhibitory compounds are CDK4 inhibitors. In some embodiments, the one or more additional CDK inhibitory compounds are CDK6 inhibitors. In some embodiments, the one or more additional CDK inhibitory compounds are CDK7 inhibitors. In some embodiments, the one or more additional CDK inhibitory compounds are CDK4/CDK6 inhibitors.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of a chemotherapeutic agent. In some embodiments, the chemotherapeutic agent is a taxane. In some embodiments, the chemotherapeutic agent is a platinum agent. In some embodiments, the chemotherapeutic agent is trastuzumab.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### EXAMPLES

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### Example 1: Synthesis of Compounds of the Disclosure

### Synthesis of (2-((S)-2,2-dimethylcyclopropane-1-carbonyl)-8-(((2-(tetrahydro-2H-pyran-4-yl)pyridin-4-yl)methoxy)methyl)-2,6-diazaspiro[3.4]octan-6-yl)(thiazol-5-yl) methanone I-45

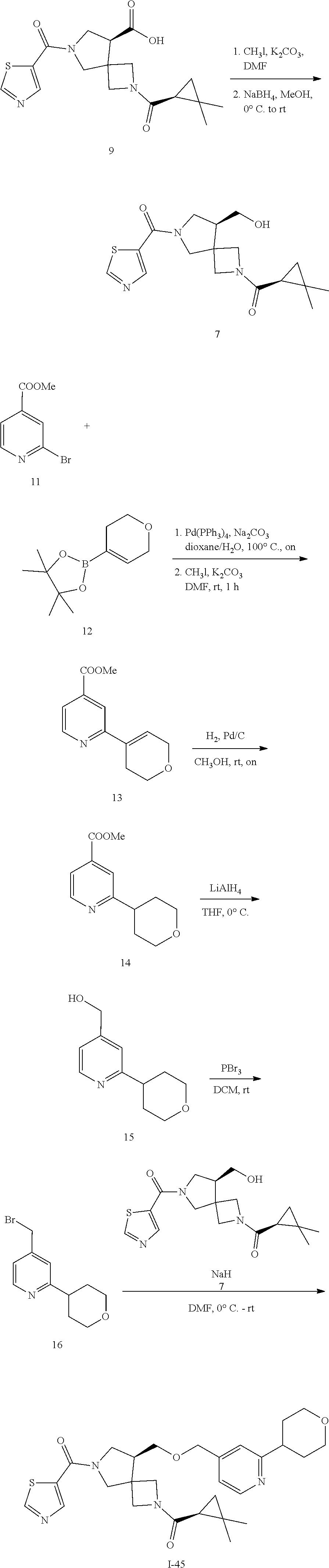

Step 1: ((S)-2-((S)-2,2-dimethylcyclopropane-1-carbonyl)-8-(hydroxymethyl)-2,6-diazaspiro[3.4]octan-6-yl)(thiazol-5-yl)methanone 7: To a mixture of (S)-2-((S)-2,2-dimethylcyclopropane-1-carbonyl)-6-(thiazole-5-carbonyl)-2,6-diazaspiro[3.4]octane-8-carboxylic acid (9) (5.000 g, 13.77 mmol) and K₂CO₃ (7.601 g, 55.080 mmol) in DMF (40 mL) was added CH₃I (5.876 g, 41.32 mmol), and the resulting mixture was stirred at room temperature for 2 hours. The reaction mixture was poured into water (100 mL) and extracted with ethyl acetate (35 mL×3). The combined organic layers were washed with brine (50 mL×2), dried over anhydrous sodium sulfate, and concentrated under reduced pressure to give crude product, which was taken up in methanol (50 mL). To the solution of methyl ester in methanol from above was added NaBH₄ (1.570 g, 41.32 mmol) in portions, and the reaction mixture was stirred at room temperature for 3 hours. The reaction mixture was poured into water (80 mL) and extracted with ethyl acetate (45 mL×3). The combined organic layers were washed with brine (50 mL×2), dried over anhydrous sodium sulfate, and concentrated under reduced pressure to give a crude residue, which was purified by silica gel column chromatography using a using a 30% ethyl acetate in hexane gradient to afford (2-((S)-2,2-dimethylcyclopropane-1-carbonyl)-8-(hydroxymethyl)-2,6-diazaspiro[3.4]octan-6-yl)(thiazol-5-yl)methanone (7) (2.900 g, 60%) as a white solid. MS: [MH]-350.1.

Step 2: Methyl 2-(3,6-dihydro-2H-pyran-4-yl)isonicotinate 13: To a suspension of methyl 2-bromoisonicotinate (11) (1.500 g, 6.90 mmol), 2-(3,6-dihydro-2H-pyran-4-yl)-4,4,5,5-tetramethyl-1,3,2-dioxaborolane (12) (2.200 g, 10.40 mmol), and Na₂CO₃ (1.473 g, 13.90 mmol) in dioxane (24 mL)-water (6 mL) was added Pd(PPh₃)₄ (0.699 g. 690 mmol) at room temperature under nitrogen atmosphere; the mixture was degassed with nitrogen three times. The resulting mixture was refluxed for 12 hours. The reaction mixture was cooled to room temperature and filtered. The filtrate was adjusted to pH 4 with hydrochloric acid (2N) and concentrated to give 2-(3,6-dihydro-2H-pyran-4-yl)isonicotinic acid (1.3 g, crude), which was taken up in DMF (15 mL) after which was added K₂CO₃ (1.752 g, 12.70 mmol) and CH₃I (1.803 g, 12.70 mmol). The resulting mixture was stirred at room temperature for 2 hours. The reaction mixture was poured into water (50 mL) and extracted with ethyl acetate (25 mL×3). The combined organic layers were washed with brine (30 mL×2), dried over anhydrous sodium sulfate, and concentrated under reduced pressure to give a crude residue, which was purified by silica gel flash chromatography using a 30% ethyl acetate in hexane gradient to afford methyl 2-(3,6-dihydro-2H-pyran-4-yl)isonicotinate (13) (1.300 g, 87%) as a yellow solid. MS: [MH]⁺ 220.3.

Step 3: Methyl 2-(tetrahydro-2H-pyran-4-yl)isonicotinate 14: To a solution of methyl 2-(3,6-dihydro-2H-pyran-4-yl)isonicotinate (13) (1.300 g, 5.90 mmol) in methanol (50 mL) was added Pd/C (10%, 0.130 g). The mixture was stirred under hydrogen atmosphere at room temperature for 2 hours. Palladium on Carbon was removed through filtration and washed with methanol (20 mL×2). The combined filtrates were concentrated under reduced pressure to afford methyl 2-(tetrahydro-2H-pyran-4-yl)isonicotinate (14) (0.912 g, 77%). MS: [MH]⁺ 222.35.

Step 4: (2-(Tetrahydro-2H-pyran-4-yl)pyridin-4-yl)methanol 15: To a suspension of LiAlH₄ (0.342 g, 9.05 mmol) in THF (10 mL) at 0° C. was added a solution of methyl 2-(tetrahydro-2H-pyran-4-yl)isonicotinate (14) (1.0 g, 4.5 mmol) in THF (20 mL) in portions. The resulting mixture was stirred at 0° C. for 2 h. The reaction mixture was quenched with water (0.4 mL), 15% sodium hydroxide solution (0.4 mL), and water (1.2 mL) subsequently at 0° C., and the resulting mixture was stirred at room temperature for 30 minutes and filtered. The filtrate was concentrated and extracted with ethyl acetate (20 mL). The organic layer was washed with water (10 mL×2) and brine (10 mL), dried over anhydrous sodium sulfate, and concentrated to give a crude residue, which was purified by silica gel column chromatography using a 5% methanol in dichloromethane gradient to afford (2-(tetrahydro-2H-pyran-4-yl)pyridin-4-yl)methanol (15) (0.435 g, 50%). MS: [MH]⁺ 194.35.

Step 5: 4-(Bromomethyl)-2-(tetrahydro-2H-pyran-4-yl)pyridine 16: To a solution of (2-(tetrahydro-2H-pyran-4-yl)pyridin-4-yl)methanol (15) (0.120 g, 0.62 mmol) in dichloromethane (5 mL) at 0° C. was added PBr₃ (0.041 g, 0.05 mmol). The resulting mixture was warmed to room temperature, and stirred for 2 hours. The reaction mixture was quenched with aqueous sodium bicarbonate solution to pH 8-10, and extracted with DCM (20 mL×3). The combined organic layer was washed with water (10 mL), dried over anhydrous sodium sulfate, and concentrated under reduced pressure to give 4-(bromomethyl)-2-(tetrahydro-2H-pyran-4-yl)pyridine (16) (0.120 g, 75%) as a yellow oil. MS: [MH]255.90.

Step 6: ((S)-2-((S)-2,2-dimethylcyclopropane-1-carbonyl)-8-(((2-(tetrahydro-2H-pyran-4-yl)pyridin-4-yl)methoxy)methyl)-2,6-diazaspiro[3.4]octan-6-yl)(thiazol-5-yl)methanone I-45: To a suspension of ((S)-2-((S)-2,2-dimethylcyclopropane-1-carbonyl)-8-(hydroxymethyl)-2,6-diazaspiro[3.4]octan-6-yl)(thiazol-5-yl)methanone (7) (0.100 g, 0.29 mmol) in DMF (2 mL) at 0° C. was added NaH (0.023 g, 0.57 mmol), and the resulting mixture was stirred at room temperature for 1 h, followed by the addition of a solution of 4-(bromomethyl)-2-(tetrahydro-2H-pyran-4-yl)pyridine (16) (0.120 g, 0.47 mmol) in DMF (0.5 mL). The resulting mixture was stirred at room temperature for 16 h. The reaction mixture was poured into water (10 mL) and extracted with ethyl acetate (5 mL×3). The combined organic layers were washed with brine (10 mL), dried over anhydrous sodium sulfate, and concentrated under reduced pressure to give a crude residue, which was purified by silica gel flash chromatography using a 5% methanol in dichloromethane gradient to afford 1-45 (0.013 g, 8.6%) as a colorless oil. ¹H NMR (400 MHz, CD₃OD): δ 9.16 (s, 1H), 8.40-8.38 (m, 2H), 7.27 (d, J=6.4 Hz, 1H), 7.23-7.21 (m, 1H), 4.63-4.61 (m, 2H), 4.49-4.37 (m, 1H), 4.28-4.15 (m, 2H), 4.09-3.96 (m, 4H), 3.91-3.78 (m, 4H), 3.69-3.53 (m, 3H), 2.96-2.93 (m, 1H), 2.91-2.73 (m, 1H), 1.88-1.77 (m, 4H), 1.45-1.37 (m, 1H), 1.18-1.06 (m, 7H), 0.78-0.71 (m, 1H). MS: [MH]⁺ 525.25.

The following compounds were prepared in a manner analogous to the procedures described above for (2-((S)-2,2-dimethylcyclopropane-1-carbonyl)-8-(((2-(tetrahydro-2H-pyran-4-yl)pyridin-4-yl)methoxy)methyl)-2,6-diazaspiro[3.4]octan-6-yl)(thiazol-5-yl)methanone (I-45).

((S)-2-((S)-2,2-dimethylcyclopropane-1-carbonyl)-8-(((4-(tetrahydro-2H-pyran-4-yl) pyridin-2-yl)methoxy)methyl)-2,6-diazaspiro[3.4]octan-6-yl)(thiazol-5-yl)methanone I-43 (0.106 g, 40%) was afforded as a pale-yellow solid. ¹H NMR (400 MHz, CD₃OD): δ 9.18 (s, 1H), 8.42-8.39 (m, 2H), 7.38 (d, J=8.4 Hz, 1H), 7.26 (d, J=5.2 Hz, 1H), 4.66-4.63 (m, 2H), 4.51-4.38 (m, 1H), 4.27-4.16 (m, 2H), 4.12-4.02 (m, 3H), 3.99-3.78 (m, SH), 3.78-3.56 (m, 3H), 2.89-2.78 (m, 2H), 1.80-1.75 (m, 4H), 1.47-1.41 (m, 1H), 1.20-1.03 (m, 7H), 0.78-0.74 (m, 1H). MS: [MH]⁺ 525.65.

((S)-2-((S)-2,2-dimethylcyclopropane-1-carbonyl)-8-(((5-(tetrahydro-2H-pyran-4-yl) pyridin-3-yl)methoxy)methyl)-2,6-diazaspiro[3.4]octan-6-yl)(thiazol-5-yl)methanone I-42 (0.012 g, 20%) was afforded as a white solid. ¹H NMR (400 MHz, CD₃OD): δ 9.16 (s, 1H), 8.37-8.34 (m, 3H), 7.70 (d, J=8.4 Hz, 1H), 7.26 (d, J=5.2 Hz, 1H), 4.61-4.58 (m, 2H), 4.46-4.32 (m, 1H), 4.24-4.11 (m, 2H), 4.05-3.91 (m, 4H), 3.86-3.73 (m, 4H), 3.62-3.51 (m, 3H), 2.87-2.69 (m, 2H), 1.84-1.75 (m, 4H), 1.47-1.34 (m, 1H), 1.17-1.01 (m, 7H), 0.79-0.71 (m, 1H). MS: [MH]-525.50.

((8S)-8-((2-((4,4-difluorocyclohexyl)oxy)-1-(6-(tetrahydro-2H-pyran-4-yl)pyridin-2-yl) ethoxy)methyl)-2-((S)-2,2-dimethylcyclopropane-1-carbonyl)-2,6-diazaspiro[3.4]octan-6-yl)(thiazol-5-yl)methanone I-13 (0.009 g, 3%) was afforded as a yellow oil. ¹HNMR (400 MHz, CD₃OD): δ 9.18 (s, 1H), 8.36-8.39 (m, 1H), 7.69-7.78 (m, 1H), 7.22-7.36 (m, 2H), 4.56-4.62 (m, 1H), 4.36-4.49 (m, 1H), 4.16-4.26 (m, 2H), 4.05-4.08 (m, 3H), 3.52-4.00 (m, 10H), 2.92-3.02 (m, 1H), 2.64-2.80 (m, 1H), 2.02-2.07 (m, 1H), 1.73-1.90 (m, 11H), 1.05-1.20 (m, 8H), 0.79-0.82 (m, 1H), 0.77-0.78 (m, 1H). [MH]⁺ 673.5.

((S)-2-((S)-2,2-dimethylcyclopropane-1-carbonyl)-8-(((6-(tetrahydro-2H-pyran-4-yl) pyridin-2-yl)methoxy)methyl)-2,6-diazaspiro[3.4]octan-6-yl)(thiazol-5-yl)methanone I-49 (0.059 g, 20%) was afforded as a white solid. ¹H NMR (400 MHz, CD₃OD): δ 9.15 (s, 1H), 8.35 (s, 1H), 7.72 (t, J=7.8 Hz, 1H), 7.31-7.25 (m, 1H), 7.20 (d, J=7.6 Hz, 1H), 4.64-4.60 (m, 2H), 4.49-4.36 (m, 1H), 4.25-4.14 (m, 2H), 4.08-3.94 (m, 4H), 3.88-3.76 (m, 4H), 3.65-3.53 (m, 3H), 2.99-2.91 (m, 1H), 2.82-2.69 (m, 1H), 1.88-1.78 (m, 4H), 1.45-1.35 (m, 1H), 1.17-1.00 (m, 7H), 0.77-0.73 (m, 1H). MS: [MH]⁺ 525.60.

((S)-2-((S)-2,2-dimethylcyclopropanecarbonyl)-8-(((3-(trifluoromethyl)benzyl)oxy) methyl)-2,6-diazaspiro[3.4]octan-6-yl)(thiazol-5-yl)methanone I-44 (0.070 g, 48%) was afforded as a light yellow oil. ¹HNMR (400 MHz, CDCl₃): δ 9.15 (s, 1H), 8.35-8.34 (m, 1H), 7.64-7.50 (m, 4H), 4.64-4.61 (m, 2H), 4.57-4.35 (m, 1H), 4.25-3.63 (m, 9H), 2.82-2.67 (m, 1H), 1.45-1.33 (m, 1H), 1.17-1.01 (m, 7H), 0.78-0.69 (m, 1H). MS: [MH]⁺508.25.

((S)-2-((S)-2,2-dimethylcyclopropane-1-carbonyl)-8-(((4-(trifluoromethyl)benzyl)oxy) methyl)-2,6-diazaspiro[3.4]octan-6-yl)(thiazol-5-yl)methanone I-41 (0.060 g, 58%) was afforded as a light yellow oily-solid. ¹HNMR (400 MHz, CDCl₃): δ 9.15 (s, 1H), 8.38-8.32 (m, 1H), 7.61 (d, J=8.0 Hz, 2H), 7.56-7.46 (m, 2H), 4.68-4.59 (m, 2H), 4.46-3.63 (m, 10H), 2.81-2.65 (m, 1H), 1.47-1.34 (m, 1H), 1.19-1.03 (m, 7H), 0.79-0.69 (m, 1H). MS: [MH]⁺ 508.55.

((S)-8-(((3,4-dichlorobenzyl)oxy)methyl)-2-((S)-2,2-dimethylcyclopropanecarbonyl)-2,6-diazaspiro[3.4]octan-6-yl)(thiazol-5-yl)methanone I-40 (0.028 g, 27%) was afforded as a colorless oil. ¹HNMR (400 MHz, CDCl₃): δ 9.15 (s, 1H), 8.35 (s, 1H), 7.49-7.45 (m, 2H), 7.28-7.22 (m, 1H), 4.57-4.34 (m, 3H), 4.24-3.66 (m, 9H), 2.80-2.66 (m, 1H), 1.45-1.33 (m, 1H), 1.17-1.01 (m, 7H), 0.80-0.71 (m, 1H). MS: [MH]⁺ 508.10.

((S)-2-((S)-2,2-dimethylcyclopropane-1-carbonyl)-8-(((6-(4-(trifluoromethoxy) cyclohexyl)pyridin-2-yl)methoxy)methyl)-2,6-diazaspiro[3.4]octan-6-yl)(thiazol-5-yl) methanone I-24 (0.022 g, 34%) was afforded as a white solid. ¹HNMR (400 MHz, CD₃OD): δ 9.17 (s, 1H), 8.35 (s, 1H), 7.20 (t, J=7.6 Hz, 1H), 7.30-7.24 (m, 1H), 7.19 (d, J=8.0 Hz, 1H), 7.62-7.59 (m, 2H), 4.49-3.60 (m, 12H), 2.79-2.70 (m, 2H), 2.21-2.19 (m, 2H), 2.01-1.98 (m, 1H), 1.75-1.60 (m, 4H), 1.45-1.34 (m, 1H), 1.17-1.09 (m, 4H), 1.05-1.00 (m, 2H), 0.78-0.72 (m, 1H). MS: [MH]⁺ 607.20.

((S)-2-((S)-2,2-dimethylcyclopropane-1-carbonyl)-8-(((3-(tetrahydro-2H-pyran-4-yl) benzyl)oxy)methyl)-2,6-diazaspiro[3.4]octan-6-yl)(thiazol-5-yl)methanone I-39 (0.040 g, 45%) was afforded as a colorless solid. ¹HNMR (400 MHz, CD₃OD): δ 9.17 (s, 1H), 8.36 (s, 1H), 7.28-7.16 (m, 4H), 4.55-4.35 (m, 3H), 4.25-4.03 (m, 5H), 3.97-3.56 (m, 8H), 2.81-2.65 (m, 2H), 1.83-1.75 (m, 4H), 1.52-1.38 (m, 1H), 1.19-1.04 (m, 7H), 0.78-0.75 (m, 1H). MS: [MH]-524.60.

((S)-8-(((1H-pyrazol-5-yl)methoxy)methyl)-2-((S)-2,2-dimethylcyclopropanecarbonyl)-2,6-diazaspiro[3.4]octan-6-yl)(thiazol-5-yl)methanone I-29 (0.040 g, 62%) was afforded as a colorless oil. ¹HNMR (400 MHz, CD₃OD): δ 9.14 (s, 1H), 8.34 (s, 1H), 7.56 (s, 1H), 6.32-6.28 (m, 1H), 4.57-4.55 (m, 2H), 4.43-4.31 (m, 1H), 4.19-3.54 (m, 9H), 2.73-2.60 (m, 1H), 1.45-1.35 (m, 1H), 1.17-1.09 (m, 6H), 1.06-1.01 (m, 1H), 0.79-0.74 (m, 1H). MS: [MH]⁺ 430.50.

((S)-2-((S)-2,2-dimethylcyclopropane-1-carbonyl)-8-(((tetrahydro-2H-pyran-2-yl) methoxy)methyl)-2,6-diazaspiro [3.4]octan-6-yl) (thiazol-5-yl) methanone I-32 (0.030 g, 12% yield) was afforded as a white oil. ¹HNMR (400 MHz, CD₃OD): δ 9.15 (s, 1H), 8.37-8.36 (m, 1H), 4.49-4.38 (m, 1H), 4.26-4.15 (m, 2H), 4.09-3.98 (m, 2H), 3.95-3.75 (m, 4H), 3.70-3.55 (m, 5H), 3.47-3.31 (m, 4H), 2.72-2.60 (m, 1H), 1.83-1.82 (m, 1H), 1.51-1.42 (m, 4H), 1.20-1.03 (m, 7H), 0.81-0.76 (m, 1H). MS: [MH]⁺ 448.70.

((S)-8-(((1-cyclopentyl-1H-pyrazol-3-yl)methoxy)methyl)-2-((S)-2,2-dimethylcyclopropane-1-carbonyl)-2,6-diazaspiro[3.4]octan-6-yl)(thiazol-5-yl)methanone I-38 (52 mg, 45% yield) was afforded as a light yellow oily-solid. ¹HNMR (400 MHz, CDCl₃): δ 9.15 (s, 1H), 8.33 (s, 1H), 7.64-7.53 (m, 1H), 6.32-6.17 (m, 1H), 4.69-4.30 (m, 4H), 4.22-3.51 (m, 9H), 2.74-2.57 (m, 1H), 2.22-1.64 (m, 8H), 1.46-1.34 (m, 1H), 1.22-0.99 (m, 7H), 0.82-0.72 (m, 1H). MS: [MH]⁺ 498.20.

((S)-8-(((1-cyclopentyl-1H-pyrazol-5-yl)methoxy)methyl)-2-((S)-2,2-dimethylcyclopropanecarbonyl)-2,6-diazaspiro[3.4]octan-6-yl)(thiazol-5-yl)methanone I-37 (0.038 g, 20%) was afforded as colorless oil. ¹HNMR (400 MHz, CD₃OD): δ 9.17 (s, 1H), 8.35 (s, 1H), 7.43 (d, J=3.8 Hz, 1H), 6.27 (d, J=7.4 Hz, 1H), 4.63 (t, J=5.2 Hz, 1H), 4.44-4.31 (m, 1H), 4.24-3.57 (m, 11H), 2.77-2.63 (m, 1H), 2.10-1.92 (m, 6H), 1.68 (d, J=19.4 Hz, 2H), 1.17-1.11 (m, 6H), 1.08-1.05 (m, 1H), 0.80-0.77 (m, 1H). MS: [MH]498.6.

((S)-2-((S)-2,2-dimethylcyclopropane-1-carbonyl)-8-(1-((6-(tetrahydro-2H-pyran-4-yl) pyridin-2-yl)methoxy)ethyl)-2,6-diazaspiro [3.4]octan-6-yl) (thiazol-5-yl) methanone I-35 (0.025 g, 26%) was afforded as an oil. ¹HNMR (400 MHz, CD₃OD): δ 9.14 (d, J=11.2 Hz, 1H), 8.36-8.13 (m, 1H), 7.78-7.62 (m, 1H), 7.40-7.17 (m, 2H), 4.77-4.68 (m, 1H), 4.61-4.41 (m, 2H), 4.30-3.71 (m, 9H), 3.65-3.54 (m, 3H), 2.97-2.91 (m, 1H), 2.58-2.43 (m, 1H), 1.94-1.75 (m, 4H), 1.37-1.29 (m, 3H), 1.17-1.04 (m, 5H), 1.03-0.85 (m, 2H), 0.78-0.65 (m, 1H). MS: [MH]⁺ 540.10.

((S)-2-((S)-2,2-dimethylcyclopropane-1-carbonyl)-8-((1-(6-(tetrahydro-2H-pyran-4-yl) pyridin-2-yl)ethoxy)methyl)-2,6-diazaspiro[3.4]octan-6-yl)(thiazol-5-yl)methanone I-14 was afforded (0.025 g, 46%). ¹H NMR (400 MHz, CD₃OD): δ 9.18 (s, 1H), 8.35-8.38 (m, 1H), 7.72-7.79 (m, 1H), 7.21-7.34 (m, 2H), 4.41-4.59 (m, 2H), 3.94-4.25 (m, 6H), 3.69-3.90 (m, 3H), 3.49-3.66 (m, 4H), 2.93-3.01 (m, 1H), 2.65-2.77 (m, 1H), 2.21 (t, J=8 Hz, 0.5H), 2.03-2.07 (m, 1H), 1.61-1.64 (m, 0.5H), 1.44-1.46 (m, 3H), 1.32 (s, 3H), 1.08-1.21 (m, 6H), 0.78-0.94 (m, 2H). MS: [MH]⁺ 539.85.

((S)-2-((S)-2,2-dimethylcyclopropane-1-carbonyl)-8-(((1-(tetrahydro-2H-pyran-4-yl) isoquinolin-3-yl)methoxy)methyl)-2,6-diazaspiro[3.4]octan-6-yl)(thiazol-5-yl)methanone I-28 (0.120 g, 60%) was afforded as white solid. ¹HNMR (400 MHz, CDCl₃): δ 9.16-9.08 (m, 1H), 8.37-8.33 (m, 1H), 8.31 (d, J=8.4 Hz, 1H), 7.89-7.78 (m, 1H), 7.72-7.65 (m, 1H), 7.65-7.57 (m, 2H), 4.80-4.72 (m, 2H), 4.53-4.38 (m, 1H), 4.31-4.15 (m, 2H), 4.15-3.68 (m, 12H), 2.86-2.72 (m, 1H), 2.23-2.08 (m, 2H), 1.86-1.76 (m, 2H), 1.43-1.32 (m, 1H), 1.16-0.97 (m, 7H), 0.76-0.70 (m, 1H). MS: [MH]⁺ 575.65.

((S)-2-((S)-2,2-dimethylcyclopropanecarbonyl)-8-(((6-phenylpyridin-2-yl)methoxy) methyl)-2,6-diazaspiro[3.4]octan-6-yl)(thiazol-5-yl)methanone I-36 (0.050 g, 34%) was afforded as a colorless oil. ¹HNMR (400 MHz, CD₃OD): δ 9.16 (s, 1H), 8.38 (d, J=2.4 Hz, 1H), 7.98 (d, J=3.2 Hz, 2H), 7.86 (t, J=7.8 Hz, 1H), 7.75 (d, J=7.8 Hz, 1H), 7.51-7.39 (m, 4H), 4.75-4.72 (m, 2H), 4.53-4.41 (m, 1H), 4.31-3.87 (m, 8H), 3.71-3.64 (m, 1H), 2.86-2.74 (m, 1H), 1.46-1.36 (m, 1H), 1.18-1.02 (m, 7H), 0.78-0.72 (m, 1H). MS: [MH]⁺ 517.60.

((S)-2-((S)-2,2-dimethylcyclopropane-1-carbonyl)-8-(((6-(4-(trifluoromethyl)phenyl) pyridin-2-yl)methoxy)methyl)-2,6-diazaspiro[3.4]octan-6-yl)(thiazol-5-yl)methanone I-34 (0.046 g, 15%) was afforded as an off-white solid. ¹H NMR (400 MHz, CD₃OD): δ 9.14 (s, 1H), 8.35 (d, J=3.2 Hz, 1H), 8.20 (d, J=5.6 Hz, 2H) 7.91-7.83 (m, 2H), 7.77 (d, J=8.4 Hz, 2H), 7.49-7.45 (m, 1H), 4.75-4.73 (m, 2H), 4.51-4.39 (m, 1H), 4.29-4.20 (m, 1H), 4.19-4.06 (m, 2H), 4.00-3.79 (m, 5H), 3.78-3.65 (m, 1H), 2.83-2.72 (m, 1H), 1.44-1.36 (m, 1H), 1.16-1.02 (m, 7H), 0.77-0.70 (m, 1H). MS: [MH]⁺ 585.65.

((S)-2-((S)-2,2-dimethylcyclopropane-1-carbonyl)-8-(((6-(2-fluoro-4-(trifluoromethyl) phenyl)pyridin-2-yl)methoxy)methyl)-2,6-diazaspiro[3.4]octan-6-yl)(thiazol-5-yl) methanone I-33 (0.030 g, 21%) was afforded as a light yellow oily-solid. ¹HNMR (400 MHz, CDCl₃): δ 9.17-9.11 (m, 1H), 8.38-8.33 (m, 1H), 8.15-8.06 (m, 1H), 7.91 (t, J=7.6 Hz, 1H), 7.78-7.73 (m, 1H), 7.65-7.56 (m, 2H), 7.55-7.46 (m, 1H), 4.78-4.70 (m, 2H), 4.53-3.63 (m, 11H), 2.87-2.70 (m, 1H), 1.45-1.34 (m, 1H), 1.18-1.04 (m, 6H), 0.79-0.71 (m, 1H). MS: [MH]⁺ 603.65.

((S)-2-((S)-2,2-dimethylcyclopropanecarbonyl)-8-(((2-methyl-3-(tetrahydro-2H-pyran-4-yl)benzyl)oxy)methyl)-2,6-diazaspiro[3.4]octan-6-yl)(thiazol-5-yl)methanone I-31 (0.013 g, 4.3%) was afforded as a white solid. ¹H NMR (400 MHz, CD₃OD): δ 9.14-9.11 (m, 1H), 8.32-8.27 (m, 1H), 7.21-7.11 (m, 3H), 4.63-4.51 (m, 2H), 4.45-4.31 (m, 1H), 4.21-4.07 (m, 2H), 4.06-3.97 (m, 3H), 3.91-3.72- (m, 4H), 3.67-3.54 (m, 4H), 3.11-3.04 (m, 1H), 2.77-2.59 (m, 1H), 2.31-2.27 (m, 3H), 1.81-1.59 (m, 4H), 1.42-1.33 (m, 1H), 1.16-1.00 (m, 7H), 0.79-0.71 (m, 1H). MS: [MH]⁺ 538.60.

((S)-2-((S)-2,2-dimethylcyclopropanecarbonyl)-8-(((2-(tetrahydro-2H-pyran-4-yl) quinolin-8-yl)oxy)methyl)-2,6-diazaspiro[3.4]octan-6-yl)(thiazol-5-yl)methanone I-30 (0.048 g, 61%) was afforded as a white solid. ¹HNMR (400 MHz, CD₃OD): δ 9.15 (d, J=10.4 Hz, 1H), 8.42-8.39 (m, 1H), 8.20-8.17 (m, 1H), 7.48-7.45 (m, 3H), 7.27-7.22 (m, 1H), 4.73-4.65 (m, 1H), 4.55-3.90 (m, 12H), 3.64-3.58 (m, 2H), 3.21-3.02 (m, 2H), 2.06-1.93 (m, 4H), 1.11-1.05 (m, 3H), 0.94-0.90 (m, 1H), 0.80 (d, J=4.4 Hz, 1H), 0.68-0.64 (m, 2H). MS: [MH]+562.10.

((S)-2-((S)-2,2-dimethylcyclopropanecarbonyl)-8-(((5-(tetrahydro-2H-pyran-4-yl) isoquinolin-1-yl)oxy)methyl)-2,6-diazaspiro[3.4]octan-6-yl)(thiazol-5-yl)methanone I-25 (0.032 g, 20%) was afforded as a colorless oil. ¹H NMR (400 MHz, CD₃OD): δ 9.16-9.13 (m, 1H), 8.38-8.36 (m, 1H), 8.09 (d, J=8.4 Hz, 1H), 8.10-7.96 (m, 1H), 7.66-7.48 (m, 3H), 4.92-4.70 (m, 2H), 4.58-4.42 (m, 1H), 4.44-4.02 (m, 6H), 3.96-3.75 (m, 3H), 3.75-3.69 (m, 2H), 3.57-3.51 (m, 1H), 3.08-3.00 (m, 1H), 1.83-1.95 (m, 4H), 1.30-1.41 (m, 1H), 0.83-1.15 (m, 7H), 0.64-0.72 (m, 1H). MS: [MH]⁺ 561.60.

((S)-2-((S)-2,2-dimethylcyclopropanecarbonyl)-8-(((8-(tetrahydro-2H-pyran-4-yl) isoquinolin-4-yl)oxy)methyl)-2,6-diazaspiro[3.4]octan-6-yl)(thiazol-5-yl)methanone I-23 (0.035 g, 29%) was afforded as a yellow solid. ¹HNMR (400 MHz, CD₃OD): δ 9.26 (s, 1H), 9.20-9.16 (m, 1H), 8.46-8.41 (m, 1H), 8.20 (d, J=10 Hz, 1H), 8.10 (d, J=8.4 Hz, 1H), 7.79-7.65 (m, 2H), 4.62-4.55 (m, 3H), 4.38-4.20 (m, 8H), 3.86-3.74 (m, 3H), 3.18-3.06 (m, 1H), 2.00-1.89 (m, 4H), 1.44-1.33 (m, 1H), 1.20-1.11 (m, 3H), 0.98-0.94 (m, 3H), 0.76-0.75 (m, 1H), 0.71-0.68 (m, 1H). MS: [MH]⁺ 561.60.

((S)-2-((S)-2,2-dimethylcyclopropane-1-carbonyl)-8-(((5-(tetrahydro-2H-pyran-4-yl) naphthalen-1-yl)oxy)methyl)-2,6-diazaspiro[3.4]octan-6-yl)(thiazol-5-yl)methanone I-22 (0.041 g, 87%) was afforded as a white solid. ¹H NMR (400 MHz, CD₃OD): δ 9.16 (d, J=18.0 Hz, 1H), 8.38-8.44 (m, 1H), 8.05-8.08 (m, 1H), 7.75-7.79 (m, 1H), 7.30-7.48 (m, 3H), 7.02 (t, J=8.6 Hz, 1H), 4.51-4.63 (m, 1H), 4.43-4.48 (m, 2H), 4.16-4.37 (m, 4H), 3.83-4.10 (m, 5H), 3.70-3.76 (m, 2H), 3.57-3.65 (m, 1H), 3.01-3.17 (m, 1H), 1.87-1.96 (m, 4H), 1.31-1.40 (m, 1H), 1.13-1.10 (m, 3H), 0.86-0.96 (m, 3H), 0.75-0.76 (m, 1H), 0.65-0.68 (m, 1H). MS: [MH]⁺ 561.05.

((S)-2-((S)-2,2-dimethylcyclopropane-1-carbonyl)-8-(((6′-(trifluoromethyl)-[2,3′-bipyridin]-6-yl)methoxy)methyl)-2,6-diazaspiro[3.4]octan-6-yl)(thiazol-5-yl)methanone I-21 (0.088 g, 77%) was afforded as a white solid. ¹HNMR (400 MHz, CD₃OD) δ 9.35 (s, 1H), 9.14 (s, 1H), 8.68-8.61 (m, 1H), 8.37-8.33 (m, 1H), 7.96-7.89 (m, 3H), 7.57-7.47 (m, 1H), 4.79-4.72 (m, 2H), 4.54-3.59 (m, 11H), 2.86-2.72 (m, 1H), 1.44-1.34 (m, 1H), 1.18-1.03 (m, 6H), 0.78-0.69 (m, 1H). MS: [MH]⁺ 586.4.

((S)-8-(([2,3′-bipyridin]-6-ylmethoxy)methyl)-2-((S)-2,2-dimethylcyclopropane-1-carbonyl)-2,6-diazaspiro[3.4]octan-6-yl)(thiazol-5-yl)methanone I-20 (0.055 g, 55%) was afforded as a white solid. ¹HNMR (400 MHz, CD₃OD): δ 9.18 (s, 1H), 9.14 (s, 1H), 8.61-8.56 (m, 1H), 8.49-8.42 (m, 1H), 8.38-8.33 (m, 1H), 7.93-7.87 (m, 1H), 7.86-7.82 (m, 1H), 7.58-7.53 (m, 1H), 7.52-7.42 (m, 1H), 4.77-4.69 (m, 2H), 4.57-3.61 (m, 11H), 2.86-2.72 (m, 1H), 1.44-1.34 (m, 1H), 1.16-1.02 (m, 6H), 0.78-0.69 (m, 1H). MS: [MH]518.4.

### Synthesis of 2-((S)-2,2-dimethylcyclopropanecarbonyl)-8-((((6-(tetrahydro-2H-pyran-4-yl)pyridin-2-yl)methyl)amino)methyl)-2,6-diazaspiro[3.4]octan-6-yl)(thiazol-5-yl) methanone I-27

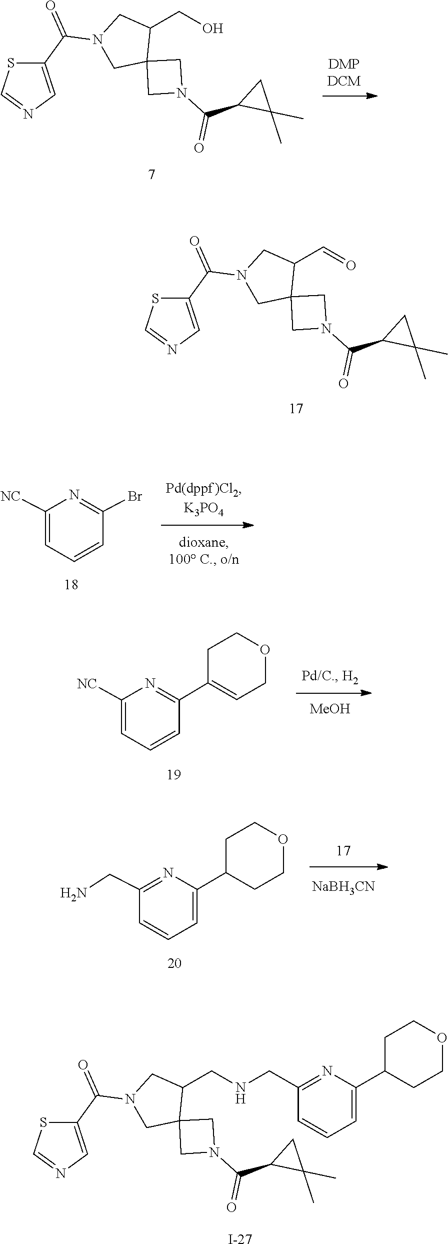

Step 1: 2-((S)-2,2-dimethylcyclopropanecarbonyl)-6-(thiazole-5-carbonyl)-2,6-diazaspiro[3.4]octane-8-carbaldehyde 17: To a solution of (2-((S)-2,2-dimethylcyclopropanecarbonyl)-8-(hydroxymethyl)-2,6-diazaspiro[3.4]octan-6-yl)(thiazol-5-yl) methanone (7) (300 mg, 0.86 mmol) in DCM (25 mL) at 0° C. was added Dess-Martin periodinane (0.729 g, 1.72 mmol). The resulting mixture was raised to 40° C. and stirred overnight. The reaction mixture was poured into water (20 mL). The organic layer was collected, washed with saturated sodium thiosulfate aqueous solution (20 mL), dried over anhydrous sodium sulfate, and concentrated under reduced pressure to give a crude residue, which was purified by silica gel column chromatography using a 2% ethyl acetate in dichloromethane gradient to afford 2-((S)-2,2-dimethylcyclopropanecarbonyl)-6-(thiazole-5-carbonyl)-2,6-diazaspiro[3.4]octane-8-carbaldehyde (17) (0.125 g, 42%) as a colorless oil. ¹HNMR (400 MHz, CDCl₃): δ 9.85 (s, 1H), 8.93 (s, 1H), 8.28-8.25- (m, 1H), 4.31-3.91 (m, 8H), 3.32 (s, 1H), 1.17 (s, 8H), 0.80-0.77 (m, 1H).

Step 2: 6-(3,6-dihydro-2H-pyran-4-yl)picolinonitrile 19: A mixture of 6-bromopicolinonitrile (18) (1.050 g, 5.50 mmol), 2-(3,6-dihydro-2H-pyran-4-yl)-4,4,5,5-tetramethyl-1,3,2-dioxaborolane (1.730 g, 8.25 mmol), Pd(dppf)Cl₂ (0.402 g, 0.55 mmol), and K₃PO₄ (2.330 g, 11.10 mmol) in dioxane (15 mL) was refluxed under nitrogen atmosphere for 2 hours. The reaction mixture was poured into water (20 mL) and extracted with ethyl acetate (30 mL). The organic layer was collected, and the aqueous layer was extracted with ethyl acetate (20 mL×2). The combined organic layers were washed with brine (40 mL), dried over anhydrous sodium sulfate, and concentrated under reduced pressure to give a crude residue, which was purified by silica gel column chromatography using a 5% ethyl acetate in hexane gradient to afford 6-(3,6-dihydro-2H-pyran-4-yl)picolinonitrile (19) (0.535 g, 52%) as a white solid. MS: [MH]-187.3.

Step 3: (6-(Tetrahydro-2H-pyran-4-yl)pyridin-2-yl)methanamine 20: To a mixture of 6-(3,6-dihydro-2H-pyran-4-yl)picolinonitrile (19) (0.535 g, 2.86 mmol) in MeOH (20 mL) was added Pd/C (10%, 0.050 g), and the resulting mixture was stirred under H₂ at room temperature for 1 hour. The reaction mixture was filtered, and the filtrate was concentrated under reduced pressure to give a crude residue, which was purified by silica gel column chromatography using a 5% methanol in dichloromethane gradient to afford (6-(tetrahydro-2H-pyran-4-yl)pyridin-2-yl)methanamine (20) (0.235 g, 52%) as a yellow oil. MS: [MH]⁺ 193.3

Step 4: 2-((S)-2,2-dimethylcyclopropanecarbonyl)-8-((((6-(tetrahydro-2H-pyran-4-yl)pyridin-2-yl)methyl)amino)methyl)-2,6-diazaspiro[3.4]octan-6-yl)(thiazol-5-yl) methanone I-27: To a mixture of (6-(tetrahydro-2H-pyran-4-yl)pyridin-2-yl)methanamine (20) (0.033 g, 0.17 mmol) and 2-((S)-2,2-dimethylcyclopropanecarbonyl)-6-(thiazole-5-carbonyl)-2,6-diazaspiro[3.4]octane-8-carbaldehyde (17) (0.060 g, 0.17 mmol) in MeOH (2 mL) was added acetic acid (0.5 mL), and the resulting mixture was stirred at room temperature for 0.5 hour, followed by the addition of NaBH₃CN (0.021 g, 0.34 mmol). The resulting mixture was stirred at room temperature overnight. The solvent was removed under reduced pressure to give a residue, which was purified by prep-HPLC to afford (2-((S)-2,2-dimethylcyclopropanecarbonyl)-8-((((6-(tetrahydro-2H-pyran-4-yl)pyridin-2-yl)methyl)amino)methyl)-2,6-diazaspiro[3.4]octan-6-yl)(thiazol-5-yl)methanone (I-27) (0.053 g, 57%) as a colorless oil. ¹HNMR (400 MHz, CD₃OD): δ 9.18 (s, 1H), 8.38 (d, J=6.0 Hz, 1H), 7.82-7.77 (m, 1H), 7.39-7.32 (m, 2H), 4.50-3.90 (m, 12H), 3.58-3.33 (m, 4H), 3.03 (q, J=12.8 Hz, 1H), 2.88-2.75 (m, 1H), 1.93-1.82 (m, 4H), 1.45-1.40 (m, 1H), 1.19-1.05 (m, 7H), 0.83-0.76 (m, 1H). MS: [MH]524.65.

I-26 was prepared in a manner analogous to the procedures described above for 2-((S)-2,2-dimethylcyclopropanecarbonyl)-8-((((6-(tetrahydro-2H-pyran-4-yl)pyridin-2-yl)methyl)amino)methyl)-2,6-diazaspiro[3.4]octan-6-yl)(thiazol-5-yl)methanone (I-27).

(2-((S)-2,2-dimethylcyclopropanecarbonyl)-8-((methyl((6-(tetrahydro-2H-pyran-4-yl)pyridin-2-yl)methyl)amino)methyl)-2,6-diazaspiro[3.4]octan-6-yl)(thiazol-5-yl) methanone I-26 (0.038 g, 54%) was afforded as a colorless oil. ¹HNMR (400 MHz, CD₃OD): δ 9.16 (d, J=5.0 Hz, 1H), 8.33 (s, 1H), 7.73-7.58 (m, 1H), 7.36-7.13 (m, 2H), 4.46-3.94 (m, 8H), 3.84-3.70 (m, 4H), 3.55 (t, J=12.0 Hz, 2H), 2.98-2.87 (m, 1H), 2.73-2.50 (m, 3H), 2.40 (d, J=14.2 Hz, 3H), 1.89-1.80 (m, 4H), 1.46-1.38 (m, 1H), 1.19-1.09 (m, 6H), 1.07-1.03 (m, 1H), 0.80-0.75 (m, 1H). MS: [MH]⁺ 538.60.

### Synthesis of (2-((tetrahydrofuran-2-yl)methyl)-8-(((6-(4-(trifluoromethyl) phenyl) pyridin-2-yl)methoxy)methyl)-2,6-diazaspiro[3.4]octan-6-yl)(thiazol-5-yl)methanone I-11

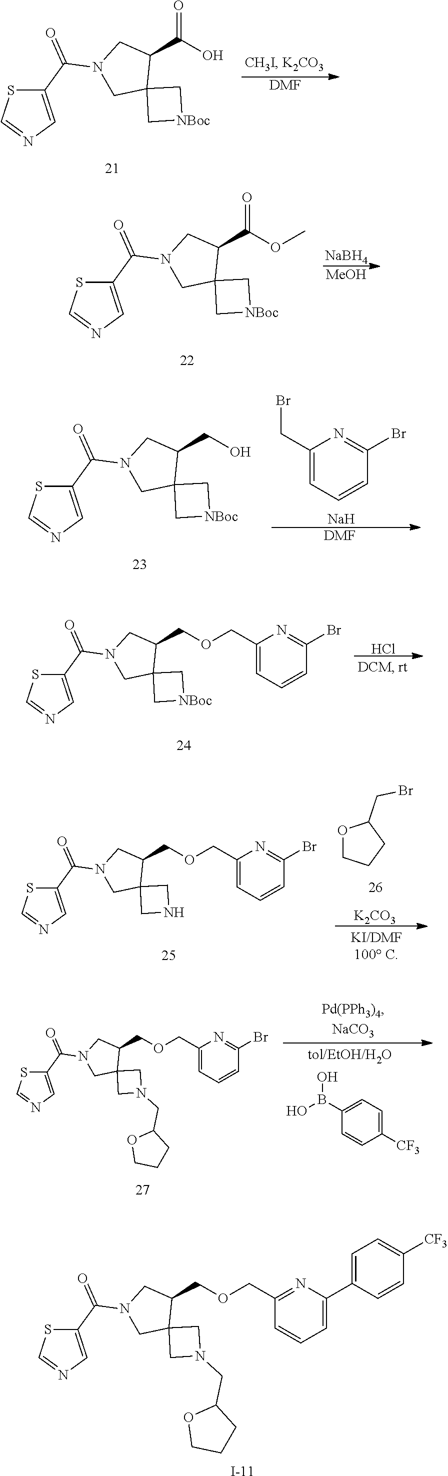

Step 1: 2-(Tert-butyl) 8-methyl (S)-6-(thiazole-5-carbonyl)-2,6-diazaspiro[3.4]octane-2,8-dicarboxylate 22: A mixture of (S)-2-(tert-butoxycarbonyl)-6-(thiazole-5-carbonyl)-2,6-diazaspiro[3.4]octane-8-carboxylic acid (21) (0.350 g, 0.95 mmol) and K₂CO₃ (0.263 g, 1.91 mmol) in DMF (5 mL) was stirred at 0° C. for 0.5 h, followed by the addition of CH₃I (0.406 g, 2.86 mmol) dropwise. The resulting mixture was stirred at room temperature for 2 hours. The reaction mixture was poured into water (50 mL) and extracted with ethyl acetate (30 mL×2). The combined organic layer was washed with brine (30 mL), dried over sodium sulfate, and concentrated under reduced pressure to give a residue, which was purified by silica gel flash chromatography using a 2% methanol in dichloromethane gradient to afford 2-(tert-butyl) 8-methyl (S)-6-(thiazole-5-carbonyl)-2,6-diazaspiro[3.4]octane-2,8-dicarboxylate (22) (0.300 g, 83%) as a white solid. MS: [MH]⁺ 325.85

Step 2: tert-butyl (S)-8-(hydroxymethyl)-6-(thiazole-5-carbonyl)-2,6-diazaspiro [3,4]octane-2-carboxylate 23: To a solution of 2-(tert-butyl) 8-methyl (S)-6-(thiazole-5-carbonyl)-2,6-diazaspiro[3.4]octane-2,8-dicarboxylate (22) (0.310 g, 0.81 mmol) in methanol (3 mL) at 0° C. was added NaBH₄ (0.185 g, 4.89 mmol) in portions. The resulting mixture was stirred at room temperature for 2 hours. The reaction mixture was poured into water (10 mL) and extracted with ethyl acetate (10 mL×2). The combined organic layer was washed with water (8 mL×2) and brine (10 mL), dried over anhydrous sodium sulfate, and concentrated under reduced pressure to give a crude residue, which was purified by silica gel flash chromatography using a 10% ethyl acetate in hexane gradient to afford tert-butyl (S)-8-(hydroxymethyl)-6-(thiazole-5-carbonyl)-2,6-diazaspiro[3.4]octane-2-carboxylate (23) (0.200 g, 70%) as a red solid. MS: [MH]⁺ 297.90.

Step 3: tert-butyl (S)-8-(((6-bromopyridin-2-yl)methoxy)methyl)-6-(thiazole-5-carbonyl)-2,6-diazaspiro[3.4]octane-2-carboxylate 24: To a solution of tert-butyl (S)-8-(hydroxymethyl)-6-(thiazole-5-carbonyl)-2,6-diazaspiro[3.4]octane-2-carboxylate (23) (0.300 g, 0.85 mmol) in THE (5 mL) at 0° C. was added NaH (0.136 g, 5.65 mmol) in portions. The resulting mixture was stirred at room temperature for 0.5 hour, followed by the addition of 2-bromo-6-(bromomethyl)pyridine (0.232 g, 0.93 mmol) dropwise. The resulting mixture was stirred at room temperature for 2 hours. The reaction mixture was poured into water (20 mL) and extracted with ethyl acetate (30 mL×3). The combined organic layer was washed with brine (40 mL), dried over anhydrous sodium sulfate, and concentrated under reduced pressure to give a crude residue, which was purified by silica gel column chromatography using a 5% methanol in dichloromethane gradient to afford tert-butyl (S)-8-(((6-bromopyridin-2-yl)methoxy)methyl)-6-(thiazole-5-carbonyl)-2,6-diazaspiro[3.4]octane-2-carboxylate (24) (0.100 g, 23%) as a yellow oil. MS: [MH]⁺ 424.55.

Step 4: (S)-(8-(((6-bromopyridin-2-yl)methoxy)methyl)-2,6-diazaspiro[3.4]octan-6-yl)(thiazol-5-yl)methanone 25: To a solution of tert-butyl (S)-8-(((6-bromopyridin-2-yl)methoxy)methyl)-6-(thiazole-5-carbonyl)-2,6-diazaspiro[3.4]octane-2-carboxylate (24) (0.230 g, 0.44 mmol) in DCM (5 mL) was added HCl in dioxane (4 M, 5 mL), and the resulting mixture was stirred at room temperature for 1 hour. The reaction mixture was concentrated under vacuum to afford tert-butyl (S)-(8-(((6-bromopyridin-2-yl)methoxy)methyl)-2,6-diazaspiro[3.4]octan-6-yl)(thiazol-5-yl)methanone (25) (0.214 g, 100%) as a yellow solid, which was used in the next step directly without purification. MS: [MH]⁺ 424.70.

Step 5: ((8S)-8-(((6-bromopyridin-2-yl)methoxy)methyl)-2-((tetrahydrofuran-2-yl)methyl)-2,6-diazaspiro[3.4]octan-6-yl)(thiazol-5-yl)methanone 27: A mixture of tert-butyl (S)-(8-(((6-bromopyridin-2-yl)methoxy)methyl)-2,6-diazaspiro[3.4]octan-6-yl)(thiazol-5-yl) methanone (25) (0.210 g, 0.50 mmol), K₂CO₃ (0.274 g, 1.99 mmol), KI (0.021 g, 0.12 mmol), and (bromomethyl)tetrahydrofuran (26) (0.274 g, 0.99 mmol) in DMF (4 mL) was stirred at 100° C. for 16 hours. The reaction mixture was poured into water (15 mL) and extracted with ethyl acetate (20 mL×3). The combined organic layers were washed with brine (40 mL), dried over anhydrous sodium sulfate, and concentrated under reduced pressure to give a crude residue, which was purified by silica gel column chromatography using a 3% methanol in dichloromethane gradient to afford ((8S)-8-(((6-bromopyridin-2-yl)methoxy)methyl)-2-((tetrahydrofuran-2-yl)methyl)-2,6-diazaspiro[3.4]octan-6-yl)(thiazol-5-yl)methanone (27) (0.100 g, 40%) as a yellow oil. MS: [MH]⁺ 508.80.

Step 6: ((8S)-2-((Tetrahydrofuran-2-yl)methyl)-8-(((6-(4-(trifluoromethyl)phenyl)pyridin-2-yl)methoxy)methyl)-2,6-diazaspiro[3.4]octan-6-yl)(thiazol-5-yl)methanone I-11: A mixture of ((8S)-8-(((6-bromopyridin-2-yl)methoxy)methyl)-2-((tetrahydrofuran-2-yl)methyl)-2,6-diazaspiro[3.4]octan-6-yl)(thiazol-5-yl)methanone (27) (0.020 g, 0.04 mmol), (4-(trifluoromethyl)phenyl)boronic acid (0.010 g, 0.06 mmol), Pd(PPh₃)₄ (0.024 g, 0.02 mmol), and Na₂CO₃ (0.024 g, 0.12 mmol) in toluene (2 mL)-EtOH (0.5 mL)-H₂O (0.5 mL) was stirred at 90° C. under N₂ atmosphere for 4 hours. The reaction mixture was cooled to room temperature, then poured into water (15 mL), and extracted with ethyl acetate (20 mL×3). The combined organic layers were washed with brine (40 mL), dried over anhydrous sodium sulfate, and concentrated under reduced pressure to give a crude residue, which was purified by silica gel column chromatography using a 10% methanol in dichloromethane gradient to afford ((8S)-2-((tetrahydrofuran-2-yl)methyl)-8-(((6-(4-(trifluoromethyl)phenyl)pyridin-2-yl)methoxy)methyl)-2,6-diazaspiro [3.4]octan-6-yl)(thiazol-5-yl)methanone (I-11) (0.016 g, 70%) as a white oil. ¹H NMR (400 MHz, CD₃OD): δ 9.10 (s, 1H), 8.17 (d, J 8.2 Hz, 3H), 7.75 (d, J 7.2 Hz, 4H), 7.64-7.37 (m, 1H), 4.38-3.55 (m, 13H), 3.16-2.82 (m, 2H), 2.68 (m, 3H), 2.05-1.70 (m, 3H), 1.62-1.40 (m, 1H). MS: [MH]⁻ 573.85.

The following compounds were prepared in a manner analogous to the procedures described above for ((8S)-2-((tetrahydrofuran-2-yl)methyl)-8-(((6-(4-(trifluoromethyl)phenyl)pyridin-2-yl)methoxy)methyl)-2,6-diazaspiro [3.4]octan-6-yl)(thiazol-5-yl)methanone (I-11).

((8S)-8-(((6-(4-cyclopropylphenyl)pyridin-2-yl)methoxy)methyl)-2-((tetrahydrofuran-2-yl)methyl)-2,6-diazaspiro[3.4]octan-6-yl)(thiazol-5-yl)methanone I-8 (0.018 g, 60%) was afforded as a white oil. ¹H NMR (400 MHz, CD₃OD): δ 9.13 (d, J 10.8 Hz, 1H), 8.33 (d, J=8.0 Hz, 1H), 7.86-7.79 (m, 3H), 7.68 (d, J=7.8 Hz, 1H), 7.36-7.29 (m, 1H), 7.16 (d, J 8.2 Hz, 2H), 4.67 (d, J 8.4 Hz, 2H), 4.11 (d, J 4.2 Hz, 1H), 3.97-3.63 (m, 9H), 3.56-3.50 (m, 1H), 3.44-3.38 (m, 2H), 2.70-2.56 (m, 3H), 1.98-1.91 (m, 2H), 1.83-1.80 (m, 2H), 1.47-1.40 (m, 1H), 1.05-0.96 (m, 2H), 0.73-0.70 (m, 2H). MS: [MH]⁺ 545.85.

((8S)-2-((tetrahydrofuran-2-yl)methyl)-8-(((6-(4-(1-(trifluoromethyl)cyclopropyl)phenyl) pyridin-2-yl)methoxy)methyl)-2,6-diazaspiro[3.4]octan-6-yl)(thiazol-5-yl)methanone I-7 (0.015 g, 31%) was afforded as a yellow oil. ¹H NMR (400 MHz, CD3OD): δ 9.13 (d, J 9.6 Hz, 1H), 8.33 (d, J 7.0 Hz, 1H), 7.99-7.96 (m, 2H), 7.86-7.82 (m, 1H), 7.75 (d, J=7.8 Hz, 1H), 7.58 (d, J=8.2 Hz, 2H), 7.41-7.36 (m, 1H), 4.70 (d, J 6.4 Hz, 2H), 4.15 (d, J 5.6 Hz, 1H), 4.02-3.50 (m, 13H), 2.84-2.60 (m, 3H), 1.98-1.76 (m, 3H), 1.41-1.38 (m, 2H), 1.13 (d, J 10.6 Hz, 2H). MS: [MH]⁺ 613.85.

(S)-(2-neopentyl-8-(((6-(4-(trifluoromethyl)phenyl)pyridin-2-yl)methoxy)methyl)-2,6-diazaspiro[3.4]octan-6-yl)(pyrazin-2-yl)methanone I-17 (0.027 g, 45%) was afforded as a yellow solid. ¹H NMR (400 MHz, CD3OD): δ 8.91-8.87 (m, 1H), 8.62-8.53 (m, 2H), 8.10 (t, J=7.6 Hz, 2H), 7.84-7.74 (m, 2H), 7.68 (d, J=4.2 Hz, 2H), 7.43-7.34 (m, 1H), 4.66-4.61 (m, 2H), 4.12-4.04 (m, 1H), 3.92-3.53 (m, 10H), 2.62-2.58 (m, 1H). 2.48-2.41 (m, 2H), 0.79-0.75 (m, 9H). MS: [MH]⁺ 554.35.

(S)-(2-neopentyl-8-(((6-(4-(trifluoromethyl)phenyl)pyridin-2-yl)methoxy)methyl)-2,6-diazaspiro[3.4]octan-6-yl)(thiazol-5-yl)methanone I-16 (0.027 g, 45%) was afforded as a yellow solid. ¹H NMR (400 MHz, CD₃OD): δ 9.04 (d, J=5.2 Hz, 1H), 8.24 (d, J=3.2 Hz, 1H), 8.10 (t, J=5.8 Hz, 2H), 7.81-7.73 (m, 2H), 7.67 (d, J=4.2 Hz, 2H), 7.39-7.33 (m, 1H), 4.62 (d, J=4.6 Hz, 2H), 4.03 (s, 1H), 3.92-3.29 (m, 9H), 2.65-2.53 (m, 1H), 2.31-2.27 (m, 2H), 0.77-0.75 (m, 9H). MS: [MH]559.35.

(S)-(2-(oxetan-3-ylmethyl)-8-(((6-(4-(trifluoromethyl)phenyl)pyridin-2-yl)methoxy) methyl)-2,6-diazaspiro[3.4]octan-6-yl)(thiazol-5-yl)methanone I-15 (0.030 g, 51%) was afforded as a white solid. ¹H NMR (400 MHz, CD₃OD): δ 9.03 (d, J=4.6 Hz, 1H), 8.23 (s, 1H), 8.10 (t, J=7.0 Hz, 2H), 7.81-7.73 (m, 2H), 7.67 (d, J=4.0 Hz, 2H), 7.37-7.31 (m, 1H), 4.65-4.59 (m, 4H), 4.29-4.24 (m, 2H), 4.03-3.54 (m, 7H), 3.46-3.39 (m, 1H), 3.30-3.25 (m, 1H), 3.18-3.16 (m, 1H), 2.97-2.87 (m, 1H), 2.76-2.70 (m, 2H), 2.61-2.50 (m, 1H). MS: [MH]-559.35.

### Synthesis of ((S)-2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octan-6-yl) (pyrazin-2-yl)methanone I-18

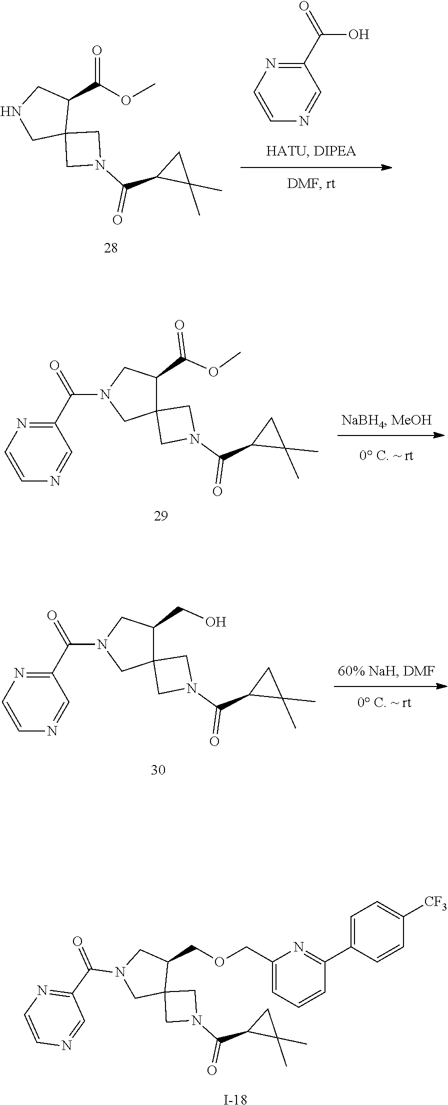

Step 1: methyl (S)-2-((S)-2,2-dimethylcyclopropane-1-carbonyl)-2,6-diazaspiro [3,4]octane-8-carboxylate (28): Compound 28 was obtained as described below in reference to the synthesis of I-19.

Step 2: methyl (S)-2-((S)-2,2-dimethylcyclopropane-1-carbonyl)-6-(pyrazine-2-carbonyl)-2,6-diazaspiro[3.4]octane-8-carboxylate 29: To a stirred solution of methyl (S)-2-((S)-2,2-dimethylcyclopropane-1-carbonyl)-2,6-diazaspiro[3.4]octane-8-carboxylate (28) (0.120 g, 0.45 mmol), pyrazine-2-carboxylic acid (0.056 g, 0.45 mmol), and N-ethyl-N-isopropylpropan-2-amine (0.174 g, 1.35 mmol) in N,N-dimethylformamide (4 mL) under nitrogen atmosphere at 0° C. was added HATU (2-(7-Aza-1H-benzotriazole-1-yl)-1,1,3,3-tetramethyluronium hexafluorophosphate) (0.257 g, 0.68 mmol). The resulting mixture was allowed to warm to room temperature and stirred for 2 hours. The reaction mixture was poured into water (7 mL) and extracted with ethyl acetate (15 mL). The organic layer was washed with water (8 mL×2) and brine (10 mL), dried over anhydrous sodium sulfate, and concentrated under reduced pressure to give a crude residue, which was purified by silica gel column chromatography using a 2% ethyl acetate in dichloromethane gradient to afford methyl (S)-2-((S)-2,2-dimethylcyclopropane-1-carbonyl)-6-(pyrazine-2-carbonyl)-2,6-diazaspiro[3.4]octane-8-carboxylate (29) (0.140 g, 72%) as a yellow oil. MS: [MH]⁺373.70.

Step 3: ((S)-2-((S)-2,2-dimethylcyclopropane-1-carbonyl)-8-(hydroxymethyl)-2,6-diazaspiro[3.4]octan-6-yl)(pyrazin-2-yl)methanone 30: To a solution of methyl (S)-2-((S)-2,2-dimethylcyclopropane-1-carbonyl)-6-(pyrazine-2-carbonyl)-2,6-diazaspiro[3.4]octane-8-carboxylate (29) (0.100 g, 0.27 mmol) in methanol (3 mL) at 0° C. was added sodium borohydride (0.102 g, 2.70 mmol) in portions. The resulting mixture was stirred at room temperature overnight. The reaction mixture was quenched with saturated aqueous ammonium chloride solution and extracted with dichloromethane (8 mL×3). The combined organic layers were washed with brine (15 mL), dried over anhydrous sodium sulfate, and concentrated under reduced pressure to give a crude residue, which was purified by preparative TLC using a 5% methanol in dichloromethane gradient to afford ((S)-2-((S)-2,2-dimethylcyclopropane-1-carbonyl)-8-(hydroxymethyl)-2,6-diazaspiro[3.4]octan-6-yl)(pyrazin-2-yl)methanone (30) (0.053 g, 57%) as a colorless oil. MS: [MH]⁺ 345.00.

Step 4: ((S)-2-((S)-2,2-dimethylcyclopropane-1-carbonyl)-8-(((6-(4-(trifluoromethyl) phenyl) pyridin-2-yl)methoxy)methyl)-2,6-diazaspiro[3.4]octan-6-yl)(pyrazin-2-yl) methanon I-18: To a solution of ((S)-2-((S)-2,2-dimethylcyclopropane-1-carbonyl)-8-(hydroxymethyl)-2,6-diazaspiro[3.4]octan-6-yl)(pyrazin-2-yl)methanone (30) (0.053 g, 0.15 mmol) in N,N-dimethylformamide (3 mL) at 0-5° C. under nitrogen atmosphere was added sodium hydride (60% in mineral oil, 0.025 g, 0.62 mmol), and the resulting mixture was stirred at room temperature for 30 minutes, followed by the addition of 2-(bromomethyl)-6-(4-(trifluoromethyl)phenyl)pyridine (0.073 g, 0.23 mmol). The resulting mixture was stirred at room temperature for 2 hours. The reaction mixture was poured into water (5 mL) and extracted with ethyl acetate (15 mL). The organic phase was washed with water (8 mL×2) and brine (10 mL), dried over anhydrous sodium sulfate, and concentrated under reduced pressure to give a crude residue, which was purified by preparative TLC using a 5% methanol in dichloromethane gradient to afford ((S)-2-((S)-2,2-dimethylcyclopropane-1-carbonyl)-8-(((6-(4-(trifluoromethyl)phenyl) pyridin-2-yl)methoxy) methyl)-2,6-diazaspiro[3.4]octan-6-yl)(pyrazin-2-yl)methanon (I-18) (0.052 g, 58%) as a white solid. ¹HNMR (400 MHz, CD₃OD): δ 9.04-8.99 (m, 1H), 8.72-8.63 (m, 2H), 8.20 (t, J=7.6 Hz, 2H), 7.92-7.82 (m, 2H), 7.77 (d, J=8.4 Hz, 2H), 7.53-7.42 (m, 1H), 4.78-4.69 (m, 2H), 4.50-3.65 (m, 10H), 2.80-2.71 (m, 1H), 1.45-1.33 (m, 1H), 1.18-1.00 (m, 7H), 0.76-0.67 (m, 1H). MS: [MH]⁺ 580.55.

### Synthesis of ((S)-2,2-dimethylcyclopropyl)((S)-6-(pyrazin-2-ylmethyl)-8-(((6-(4-(trifluoromethyl)phenyl)pyridin-2-yl)methoxy)methyl)-2,6-diazaspiro[3.4]octan-2-yl) methanone I-19

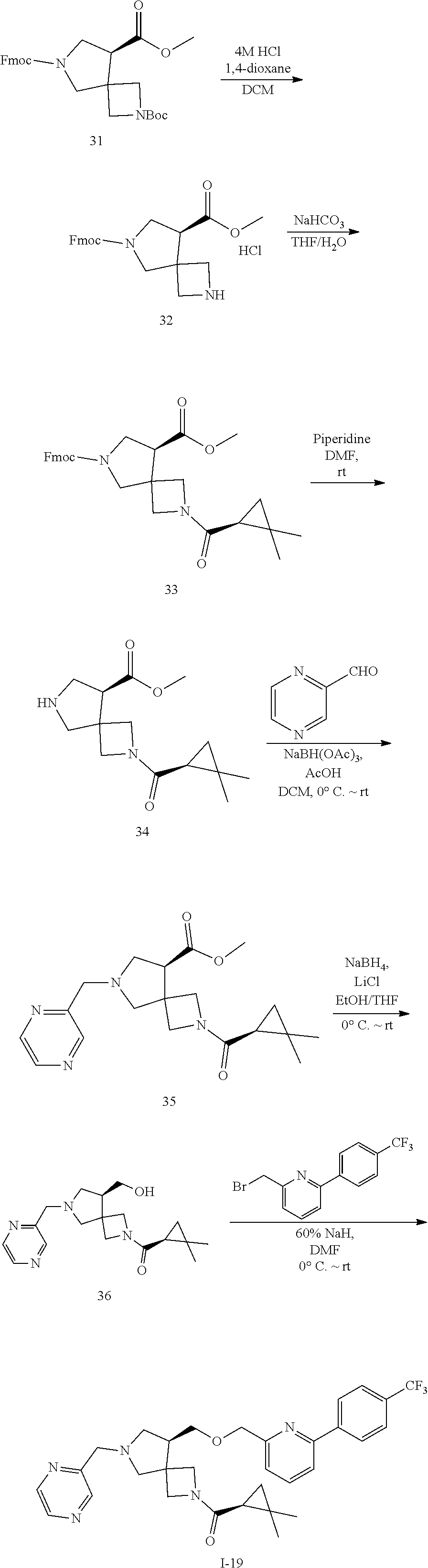

Step 1: 6-((9H-fluoren-9-yl)methyl) 8-methyl (S)-2,6-diazaspiro[3.4]octane-6,8-dicarboxylate hydrochloride 32: A mixture of 6-((9H-fluoren-9-yl)methyl) 2-(tert-butyl) 8-methyl (S)-2,6-diazaspiro[3.4]octane-2,6,8-tricarboxylate (31) (1.00 g, 2.02 mmol) and hydrogen chloride (4 M in 1,4-dioxane, 5 mL) in dichloromethane (10 mL) was stirred at room temperature for 1 hour. The volatiles were removed under reduced pressure to afford 6-((9H-fluoren-9-yl)methyl) 8-methyl (S)-2,6-diazaspiro[3.4]octane-6,8-dicarboxylate hydrochloride (32) (crude 1.0 g) as white solid, which was used in the next step without further purification. MS: [MH]-393.93

Step 2: 6-((9H-fluoren-9-yl)methyl) 8-methyl (S)-2-((S)-2,2-dimethylcyclopropane-1-carbonyl)-2,6-diazaspiro[3.4]octane-6,8-dicarboxylate 33: A mixture of 2,5-dioxopyrrolidin-1-yl (S)-2,2-dimethylcyclopropane-1-carboxylate (0.532 g, 2.52 mmol), 6-((9H-fluoren-9-yl)methyl) 8-methyl (S)-2,6-diazaspiro[3.4]octane-6,8-dicarboxylate hydrochloride (32) (0.900 g, 2.29 mmol), and sodium bicarbonate (0.769 g, 9.16 mmol) in tetrahydrofuran-water (8 mL-8 mL) was stirred at room temperature for 30 minutes. The reaction mixture was extracted with ethyl acetate (10 mL×2) to remove impurities; the aqueous layer was acidified with diluted hydrochloric acid (1N) to a pH of 3-4, and extracted with dichloromethane (15 mL×3). The combined organic layers were washed with brine (20 mL), dried over sodium sulfate, and concentrated under reduced pressure to afford 6-((9H-fluoren-9-yl)methyl) 8-methyl (S)-2-((S)-2,2-dimethylcyclopropane-1-carbonyl)-2,6-diazaspiro[3.4]octane-6,8-dicarboxylate (33) (0.870 g, 100%) as a colorless oily-solid. MS: [MH]⁺ 489.85.

Step 3: methyl(S)-2-((S)-2,2-dimethylcyclopropane-1-carbonyl)-2,6-diazaspiro[3.4]octane-8-carboxylate 34: A mixture of 6-((9H-fluoren-9-yl)methyl) 8-methyl (S)-2-((S)-2,2-dimethylcyclopropane-1-carbonyl)-2,6-diazaspiro[3.4]octane-6,8-dicarboxylate (33) (0.740 g) and piperidine (3 mL) in N,N-dimethylformamide (12 mL) was stirred at room temperature for 1 hour. The volatiles were removed under reduced pressure to give a crude residue, which was purified by silica gel column chromatography using a 10% methanol in dichloromethane to afford methyl(S)-2-((S)-2,2-dimethylcyclopropane-1-carbonyl)-2,6-diazaspiro[3.4]octane-8-carboxylate (34) (0.368 mg, 91%) as a light-yellow oil. MS: [MH]267.60.

Step 4: methyl (S)-2-((S)-2,2-dimethylcyclopropane-1-carbonyl)-6-(pyrazin-2-ylmethyl)-2,6-diazaspiro[3.4]octane-8-carboxylate 35: To a mixture of methyl(S)-2-((S)-2,2-dimethylcyclopropane-1-carbonyl)-2,6-diazaspiro[3.4]octane-8-carboxylate (34) (0.100 g, 0.38 mmol) and acetic acid (1 drop) in dichloromethane (3 mL) was added pyrazine-2-carbaldehyde (0.49 g, 0.45 mmol). The resulting mixture was stirred at room temperature for 30 minutes, followed by the addition of sodium triacetoxyborohydride (0.398 g, 1.88 mmol). The resulting mixture was stirred at room temperature for 1 hour. The reaction mixture was poured into half-saturated aqueous sodium bicarbonate solution (5 mL) and extracted with dichloromethane (8 mL×2). The combined organic layers were washed with brine (10 mL), dried over anhydrous sodium sulfate, and concentrated under reduced pressure to give a crude residue, which was purified by preparative TLC using a 5% methanol in dichloromethane gradient to afford methyl (S)-2-((S)-2,2-dimethylcyclopropane-1-carbonyl)-6-(pyrazin-2-ylmethyl)-2,6-diazaspiro[3.4]octane-8-carboxylate (35) (0.110 g, 81%) as a yellow oil. MS: [MH]⁺ 359.35.

Step 5: ((S)-2,2-dimethylcyclopropyl)((S)-8-(hydroxymethyl)-6-(pyrazin-2-ylmethyl)-2,6-diazaspiro[3.4]octan-2-yl)methanone 36: To a solution of sodium borohydride (0.044 g, 1.16 mmol) in ethanol (2 mL) at 0° C. under nitrogen atmosphere was added lithium chloride (0.049.1 mg, 1.16 mmol). The resulting mixture was stirred at 0° C. for 10 minutes, followed by the addition of methyl (S)-2-((S)-2,2-dimethylcyclopropane-1-carbonyl)-6-(pyrazin-2-ylmethyl)-2,6-diazaspiro[3.4]octane-8-carboxylate (35) (0.083 g, 0.23 mmol) in tetrahyfuran (2 mL). The resulting mixture was allowed to warm up to room temperature and stirred overnight. The reaction mixture was quenched with saturated aqueous ammonium chloride solution (5 mL) and extracted with ethyl acetate (8 mL×3). The combined organic layers were washed with brine (15 mL), dried over anhydrous sodium sulfate, and concentrated under reduced pressure to give a crude residue, which was purified by preparative TLC using a 5% methanol in dichloromethane gradient to afford ((S)-2,2-dimethylcyclopropyl)((S)-8-(hydroxymethyl)-6-(pyrazin-2-ylmethyl)-2,6-diazaspiro [3.4]octan-2-yl)methanone (36) (0.035 g, 46%) as a light-yellow oil. MS: [MH]-330.85.

Step 6: ((S)-2,2-dimethylcyclopropyl)((S)-6-(pyrazin-2-ylmethyl)-8-(((6-(4-(trifluoromethyl)phenyl)pyridin-2-yl)methoxy)methyl)-2,6-diazaspiro[3.4]octan-2-yl) methanone I-19: To a solution of ((S)-2,2-dimethylcyclopropyl)((S)-8-(hydroxymethyl)-6-(pyrazin-2-ylmethyl)-2,6-diazaspiro[3.4]octan-2-yl)methanone (36) (0.032 g, 0.097 mmol) in N,N-dimethylformamide (2 mL) at 0-5° C. under nitrogen atmosphere was added sodium hydride (60% in mineral oil) (0.015 g, 0.39 mmol). The resulting mixture was stirred at room temperature for 30 minutes, followed by the addition of 2-(bromomethyl)-6-(4-(trifluoromethyl)phenyl)pyridine (0.046 g, 0.15 mmol). The resulting mixture was stirred at room temperature for 2 hours. The reaction mixture was poured into water (5 mL) and extracted with ethyl acetate (15 mL). The organic layer was washed with water (8 mL×2) and brine (10 mL), dried over anhydrous sodium sulfate, and concentrated under reduced pressure to give a crude residue, which was purified by preparative TLC using a 5% methanol in dichloromethane gradient to afford ((S)-2,2-dimethylcyclopropyl)((S)-6-(pyrazin-2-ylmethyl)-8-(((6-(4-(trifluoromethyl) phenyl)pyridin-2-yl)methoxy)methyl)-2,6-diazaspiro[3.4]octan-2-yl)methanone (I-19) (0.018 g, 33%) as a colorless oil. ¹HNMR (400 MHz, CD₃OD): δ 8.70-8.67 (m, 1H), 8.59-8.56 (m, 1H), 8.53-8.49 (m, 1H), 8.21 (d, J=8.0 Hz, 2H), 7.94-7.88 (m, 1H), 7.87-7.82 (m, 1H), 7.78 (d, J=8.4 Hz, 2H), 7.47 (t, J=7.6 Hz, 1H), 4.74-4.68 (m, 2H), 4.48-3.70 (m, 8H), 3.11-3.00 (m, 2H), 2.90-2.79 (m, 1H), 2.65-2.55 (m, 1H), 2.51-2.42 (m, 1H), 1.40-1.34 (m, 1H), 1.16-0.94 (m, 7H), 0.75-0.68 (m, 1H). MS: [MH]⁺ 566.70.

The following compounds were prepared in a manner analogous to the procedures described above for ((S)-2,2-dimethylcyclopropyl)((S)-6-(pyrazin-2-ylmethyl)-8-(((6-(4-(trifluoromethyl)phenyl)pyridin-2-yl)methoxy)methyl)-2,6-diazaspiro[3.4]octan-2-yl)methanone (I-19).

((R)-2,2-difluorocyclopropyl)((S)-6-(thiazol-5-ylmethyl)-8-(((6-(4-(trifluoromethyl) phenyl)pyridin-2-yl)methoxy)methyl)-2,6-diazaspiro[3.4]octan-2-yl)methanone I-12 (0.088 g, 43%) was afforded as a white solid. ¹HNMR (400 MHz, CD₃OD): δ 8.94 (s, 1H), 8.20 (d, J=7.2 Hz, 2H), 7.91-7.84 (m, 2H), 7.88-7.87 (m, 3H), 7.45-7.34 (m, 1H), 4.72-4.70 (m, 2H), 4.58-4.52 (m, 1H), 4.33-4.25 (m, 1H), 4.07-4.00 (m, 1H), 3.93 (s, 2H), 3.74-3.72 (m, 2H), 3.03-3.00 (m, 2H), 2.83-2.76 (m, 1H), 2.59-2.57 (m, 2H), 2.45-2.40 (m, 1H), 2.04-1.59 (m, 3H). MS: [MH]⁺ 579.50.

N-(((8R)-2-((S)-2,2-dimethylcyclopropane-1-carbonyl)-6-((tetrahydrofuran-2-yl) methyl)-2,6-diazaspiro[3.4]octan-8-yl)methyl)-6-(2-methoxyphenyl)picolinamide I-10 (0.062 g, 30%) was afforded as an off-white solid. ¹HNMR (400 MHz, CD₃OD): δ 8.08-7.98 (m, 2H), 7.97-7.91 (m, 1H), 7.91-7.82 (m, 1H), 7.43 (d, J=8.0 Hz, 1H), 7.17-7.07 (m, 2H), 4.45-3.68 (m, 11H), 3.63-3.52 (m, 1H), 3.21-3.04 (m, 3H), 2.78-2.60 (m, 4H), 2.00-1.78 (m, 3H), 1.56-1.44 (m, 1H), 1.10-1.13 (m, 1H), 1.11-0.84 (m, 7H), 0.75-0.52 (m, 1H). MS: [MH]⁺ 533.75.

N-(((8R)-2-((S)-2,2-dimethylcyclopropane-1-carbonyl)-6-((tetrahydrofuran-2-yl) methyl)-2,6-diazaspiro[3.4]octan-8-yl)methyl)-6-(4-(trifluoromethyl)phenyl)picolinamide 1-9 (0.066 g, 73%) was afforded as an off-white solid. ¹HNMR (400 MHz, CD₃OD): δ 8.44-8.35 (m, 2H), 8.20-8.06 (m, 3H), 7.82 (d, J=8.0 Hz, 2H), 4.48-3.59 (m, 9H), 3.28-3.08 (m, 3H), 2.84-2.65 (m, 4H), 2.05-1.95 (m, 1H), 1.92-1.80 (m, 2H), 1.60-1.48 (m, 1H), 1.41-1.16 (m, 1H), 1.12-0.82 (m, 7H), 0.75-0.43 (m, 1H). MS: [MH]⁺ 571.65.

### Synthesis of 4-((4-(2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octane-6-carbonyl)-1H-pyrazol-1-yl)methyl)benzoic acid I-6

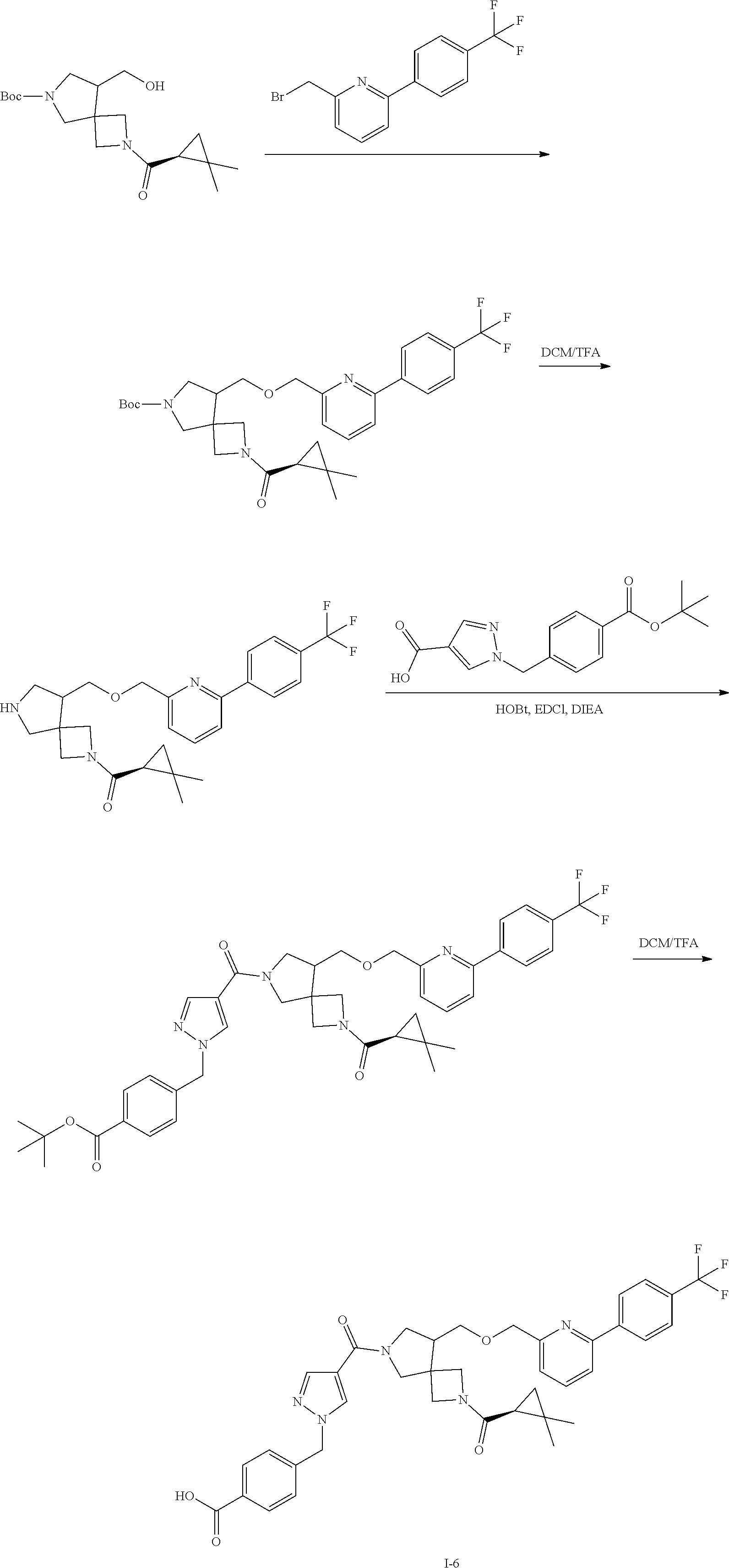

Step 1: tert-butyl 2-((S)-2,2-dimethylcyclopropane-1-carbonyl)-8-(((6-(4-(trifluoromethyl) phenyl)pyridin-2-yl)methoxy)methyl)-2,6-diazaspiro [3.4]octane-6-carboxylate: To a solution of tert-butyl 2-((S)-2,2-dimethylcyclopropane-1-carbonyl)-8-(hydroxymethyl)-2,6-diazaspiro[3.4]octane-6-carboxylate (180 mg, 0.53 mmol) in dry DMF (2.0 mL) was added NaH (32 mg, 0.80 mmol). The mixture was stirred at 0° C. for 30 minutes, and then 2-(bromomethyl)-6-(4-(trifluoromethyl)phenyl)pyridine (185 mg, 0.59 mmol) was added. The reaction mixture was stirred at room temperature overnight. The reaction was quenched with water (25 mL) and extracted with EtOAc (50 mL×3). The combined organic layers were washed with brine, dried over Na₂SO₄, filtered, and concentrated. The residue was purified by prep-TLC (eluent: DCM/MeOH=20:1) to afford tert-butyl 2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octane-6-carboxylate (80 mg, 35%) as a yellow oil. LCMS m/z=574.3 [M+H]⁺. ¹H NMR (400 MHz, CD3OD): δ 8.21 (d, J=8.2 Hz, 2H), 7.95-7.89 (m, 1H), 7.85 (d, J=7.9 Hz, 1H), 7.78 (d, J=8.2 Hz, 2H), 7.48 (t, J=6.7 Hz, 1H), 4.75-4.70 (m, 2H), 4.28-3.46 (m, 10H), 3.35 (s, 5H), 2.64 (s, 1H), 1.46 (s, 9H), 1.16-1.07 (m, 5H), 1.05-1.02 (m, 2H), 0.81-0.66 (m, 1H).

Step 2: ((S)-2,2-dimethylcyclopropyl)(8-(((6-(4-(trifluoromethyl)phenyl)pyridin-2-yl)methoxy)methyl)-2,6-diazaspiro[3.4]octan-2-yl)methanone: To a solution of tert-butyl 2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octane-6-carboxylate (80 mg, 0.14 mmol) in DCM (1.0 mL) was added TFA (0.5 mL), and the reaction was stirred at room temperature for 1 hour. The solvent was removed under vacuum to afford ((S)-2,2-dimethylcyclopropyl)(8-(((6-(4-(trifluoromethyl)phenyl)pyridin-2-yl)methoxy)methyl)-2,6-diazaspiro[3.4]octan-2-yl)methanone (66 mg, quant.) as a yellow oil. LCMS m/z=474.2 [M+H]⁺. ¹H NMR (400 MHz, DMSO-d₆): δ 8.85 (s, 2H), 8.30 (d, J=8.2 Hz, 2H), 8.03-7.93 (m, 2H), 7.86 (d, J=8.2 Hz, 2H), 7.47 (d, J=7.2 Hz, 1H), 4.70 (s, 2H), 3.10 (s, 1H), 2.71-2.62 (m, 1H), 2.06-1.92 (m, 1H), 1.35-1.18 (m, 7H), 1.12-0.99 (m, 5H), 0.96 (s, 2H), 0.70-0.58 (m, 1H).

Step 3: tert-butyl 4-((4-(2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octane-6-carbonyl)-1H-pyrazol-1-yl)methyl)benzoate: To a solution of 1-(4-(tert-butoxycarbonyl)benzyl)-1H-pyrazole-4-carboxylic acid (51 mg, 0.17 mmol) in DCM (1 mL) was added EDCI (40 mg, 0.21 mmol), HOBt (28 mg, 0.21 mmol), and DIPEA (54 mg, 0.42 mmol). The mixture was stirred for 30 minutes. ((S)-2,2-dimethylcyclopropyl)(8-(((6-(4-(trifluoromethyl)phenyl)pyridin-2-yl)methoxy)methyl)-2,6-diazaspiro[3.4]octan-2-yl)methanone (66 mg, 0.14 mmol) was added, and the reaction mixture was stirred at room temperature overnight. The reaction mixture was diluted with water (15 mL) and extracted with DCM (30 mL×2). The combined organic layers were washed with brine, dried over Na₂SO₄, filtered, and concentrated. The residue obtained was purified by prep-TLC (eluent: DCM/MeOH=15:1) to afford tert-butyl 4-((4-(2-((S)-2,2-dimethylcyclopropane-1-carbonyl)-8-(((6-(4-(trifluoromethyl)phenyl)pyridin-2-yl)methoxy)methyl)-2,6-diazaspiro[3.4]octane-6-carbonyl)-1H-pyrazol-1-yl)methyl)benzoate (45 mg, 45%) as a white solid. LCMS m/z=758.2 [M+H]⁺. H NMR (400 MHz, CD₃OD): δ 8.25 (d, J=9.0 Hz, 1H), 8.18 (d, J=7.8 Hz, 2H), 7.96-7.89 (m, 3H), 7.87-7.79 (m, 2H), 7.76 (d, J=8.2 Hz, 2H), 7.47-7.38 (m, 1H), 7.31 (t, J=7.7 Hz, 2H), 5.49 (s, 1H), 5.44 (s, 2H), 4.75-4.69 (m, 2H), 4.60-3.57 (m, 11H), 2.84-2.65 (m, 1H), 1.46-1.34 (m, 1H), 1.16-0.97 (m, 7H), 0.78-0.68 (m, 1H).

Step 4: 4-((4-(2-((S)-2,2-dimethylcyclopropane-1-carbonyl)-8-(((6-(4-(trifluoromethyl) phenyl)pyridin-2-yl)methoxy)methyl)-2,6-diazaspiro[3.4]octane-6-carbonyl)-1H-pyrazol-1-yl)methyl)benzoic acid (I-6): To a solution of tert-butyl 4-((4-(2-((S)-2,2-dimethylcyclopropane-1-carbonyl)-8-(((6-(4-(trifluoromethyl)phenyl)pyridin-2-yl)methoxy) methyl)-2,6-diazaspiro[3.4]octane-6-carbonyl)-1H-pyrazol-1-yl)methyl)benzoate (30 mg, 0.04 mmol) in DCM (1.0 mL) was added TFA (0.5 mL), and the reaction was stirred for 1 hour. The solvent was removed under reduced pressure, and the residue obtained was purified by RP-column to give 4-((4-(2-((S)-2,2-dimethylcyclopropane-1-carbonyl)-8-(((6-(4-(trifluoromethyl)phenyl) pyridin-2-yl)methoxy)methyl)-2,6-diazaspiro[3.4]octane-6-carbonyl)-1H-pyrazol-1-yl)methyl) benzoic acid (25 mg, 92%) as a white solid. LCMS m/z=702.1 [M+H]⁺. ¹H NMR (400 MHz, CD₃OD): δ 8.24 (d, 1H), 8.18 (d, 2H), 8.02-7.91 (m, 3H), 7.85-7.73 (m, 4H), 7.46-7.37 (m, 1H), 7.33 (t, 2H), 5.45 (d, J=3.6 Hz, 2H), 4.76-4.67 (m, 2H), 4.60-3.57 (m, 11H), 2.83-2.63 (m, 1H), 1.43-1.33 (m, 1H), 1.17-0.96 (m, 7H), 0.79-0.66 (m, 1H).

### Synthesis of methyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate 1-5

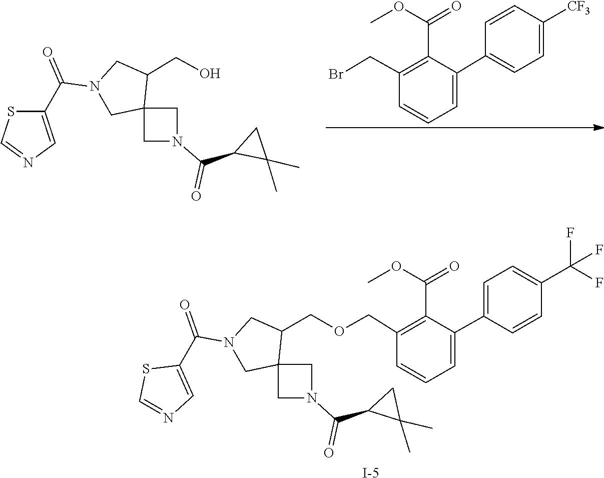

To a solution of (2-((S)-2,2-dimethylcyclopropane-1-carbonyl)-8-(hydroxymethyl)-2,6-diazaspiro[3.4]octan-6-yl)(thiazol-5-yl)methanone (30 mg, 85.9 mol) in THF (2 mL) at 0° C. was added NaH (3 mg, 128.9 mol). The mixture was stirred for 30 minutes, and then methyl 3-(bromomethyl)-4′-(trifluoromethyl)-[1,1′-biphenyl]-2-carboxylate (30 mg, 85.9 μmol) was added. The reaction was allowed to warm to room temperature and stirred for 2 hours. Next, the reaction mixture was diluted with water (20 mL) and extracted with EtOAc (30 mL×2). The combined organic layers were washed with brine, dried over Na₂SO₄, filtered, and concentrated. The residue was purified by RP-column to afford methyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate (20 mg, 36%). LCMS m/z=642.1 [M+H]⁺. ¹HNMR (400 MHz, CDCl₃): δ 9.14 (s, 1H), 8.32 (d, J=18.1 Hz, 1H), 7.74-7.69 (m, 2H), 7.50 (d, J=9.5 Hz, 4H), 7.40 (d, J=7.2 Hz, 1H), 4.71-4.66 (m, 2H), 4.51-3.68 (m, 10H), 3.57 (d, J=11.2 Hz, 3H), 2.71-2.57 (m, 1H), 1.42-1.39 (m, 1H), 1.18-1.01 (m, 7H), 0.79-0.69 (m, 1H).

### Synthesis of 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid I-4

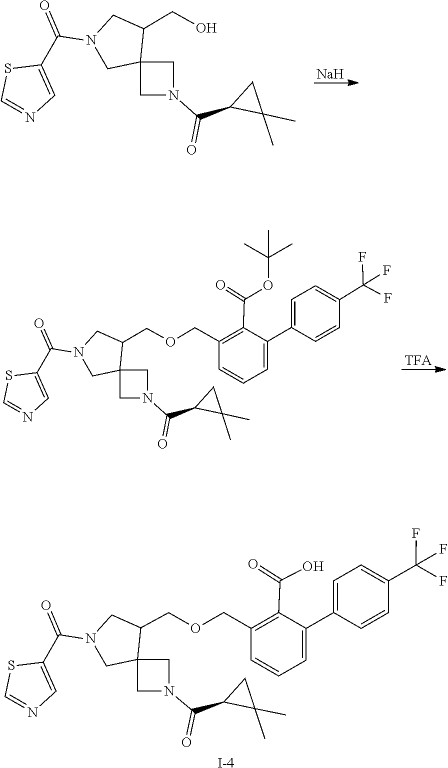

Step 1: tert-butyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate: To a solution of (2-((S)-2,2-dimethylcyclopropane-1-carbonyl)-8-(hydroxymethyl)-2,6-diazaspiro[3.4]octan-6-yl)(thiazol-5-yl)methanone (65 mg, 0.15 mmol) in THE (2 mL) at 0° C. was added NaH (4 mg, 0.17 mmol), and the mixture was stirred for 30 minutes. tert-Butyl 3-(bromomethyl)-4′-(trifluoromethyl)-[1,1′-biphenyl]-2-carboxylate (50 mg, 0.14 mmol, 1.0 eq) was added, and the reaction was allowed to warm to room temperature and stirred for 2 hours. The mixture was diluted with water (20 mL) and extracted with EtOAc (30 mL×2). The combined organic layers were washed with brine, dried over Na₂SO₄, filtered, and concentrated. The residue obtained was purified by RP-column to afford tert-butyl 3-(((2-((S)-2,2-dimethylcyclopropane-1-carbonyl)-6-(thiazole-5-carbonyl)-2,6-diazaspiro[3.4]octan-8-yl) methoxy)methyl)-4′-(trifluoromethyl)-[1,1′-biphenyl]-2-carboxylate (20 mg, 20%) as a white solid. LCMS m/z=684.5 [M+H]⁺. ¹H NMR (400 MHz, Chloroform-d): δ 8.95 (s, 1H), 8.28 (d, J=15.2 Hz, 1H), 7.70 (d, J=8.0 Hz, 2H), 7.57-7.42 (m, 4H), 7.32-7.30 (m, 1H), 4.70 (d, J=6.0 Hz, 2H), 4.49-3.46 (m, 10H), 2.75-2.60 (m, 1H), 1.40-1.10 (m, 17H), 0.79 (s, 1H).

Step 2: 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid: To a solution of tert-butyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate (50 mg, 0.07 mmol) in DCM (2.0 mL) was added TFA (2 mL), and the reaction was stirred for 2 hours. The solvent was removed under reduced pressure, and the residue was purified by RP-column to afford 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid (I-4) (20 mg, 44%) as a white solid. LCMS m/z=628.1 [M+H]⁺. ¹H NMR (400 MHz, Methanol-d₄) δ 9.13 (s, 1H), 8.32 (d, J=12.3 Hz, 1H), 7.70 (d, J=8.0 Hz, 2H), 7.59 (s, 2H), 7.48 (s, 2H), 7.37 (s, 1H), 4.70 (s, 2H), 4.59-3.52 (m, 10H), 2.76-2.58 (m, 1H), 1.47-1.35 (m, 1H), 1.21-1.04 (m, 7H), 0.75 (s, 1H). An alternative batch was prepared which yielded the following data: ¹H NMR (400 MHz, Methanol-d₄) δ 9.13 (s, 1H), 8.32 (d, J=12.3 Hz, 1H), 7.70 (d, J=8.0 Hz, 2H), 7.59 (s, 2H), 7.48 (s, 2H), 7.37 (s, 1H), 4.70 (s, 2H), 4.59-3.52 (m, 10H), 2.76-2.58 (m, 111), 1.47-1.35 (m, 1H), 1.21-1.04 (m, 7H), 0.75 (s, 1H). m/z=628.1 [M+H]⁺

### Synthesis of Building Block: Methyl 3-(bromomethyl)-4′-(trifluoromethyl)-[1,1′-biphenyl]-2-carboxylate

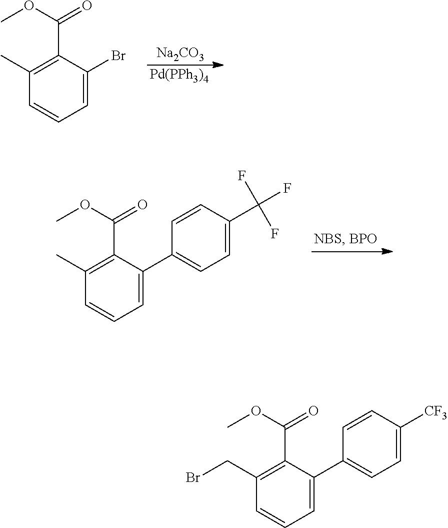

Step 1: Methyl 3-methyl-4′-(trifluoromethyl)-[1,1′-biphenyl]-2-carboxylate: To a solution of methyl 2-bromo-6-methylbenzoate (1 g, 4.4 mmol) in a mixture of DCE and water (10 mL/2.5 mL) was added (4-(trifluoromethyl)phenyl)boronic acid (916 mg, 4.8 mmol), Pd(PPh₃)₄ (506 mg, 0.44 mmol), and Na₂CO₃ (1.1 g, 8.8 mmol). The reaction was heated at 100° C. under a N₂ atmosphere for 6 hours. The reaction mixture was then diluted with water (100 mL) and extracted with EtOAc (150 mL×2). The combined organic layers were washed with brine, dried over Na₂SO₄, fitered, and concentrated. The residue was purified by column chromatography on silica gel (eluent: petroleum ether/EtOAc=20:1 to 15:1) to afford methyl 3-methyl-4′-(trifluoromethyl)-[1,1′-biphenyl]-2-carboxylate (1.2 g, 93%) as a colorless oil. ¹H NMR (400 MHz, CD₃OD): δ 7.70 (d, J=8.0 Hz, 2H), 7.51 (d, J=8.0 Hz, 2H), 7.43 (t, J=7.7 Hz, 1H), 7.32 (d, J=7.7 Hz, 1H), 7.25 (d, J=7.6 Hz, 1H), 3.59 (s, 3H), 2.38 (s, 3H).

Step 2: Methyl 3-(bromomethyl)-4′-(trifluoromethyl)-[1,1′-biphenyl]-2-carboxylate: To a solution of methyl 3-methyl-4′-(trifluoromethyl)-[1,1′-biphenyl]-2-carboxylate (100 mg, 0.34 mmol) in CCl₄ (2 mL) was added NBS (60 mg, 0.34 mmol) and BPO (16 mg, 0.07 mmol). The reaction was heated at 80° C. overnight. The reaction mixture was then diluted with water (30 mL) and extracted with EtOAc (60 mL×2). The combined organic layers were washed with brine, dried over Na₂SO₄, filtered, and concentrated. The residue was purified by column chromatography on silica gel (eluent: petroleum ether/EtOAc=40:1 to 20:1) to afford methyl 3-(bromomethyl)-4′-(trifluoromethyl)-[1,1′-biphenyl]-2-carboxylate (40 mg, 32%) as a white solid. ¹H NMR (400 MHz, CD₃OD) δ 7.73 (d, J=7.9 Hz, 2H), 7.59-7.48 (m, 4H), 7.42 (dd, J=6.1, 2.9 Hz, 1H), 4.72 (d, J=2.9 Hz, 2H), 3.59 (d, J=3.2 Hz, 3H).

### Synthesis of Building Block: tert-Butyl 3-(bromomethyl)-4′-(trifluoromethyl)-[1,1′-biphenyl]-2-carboxylate

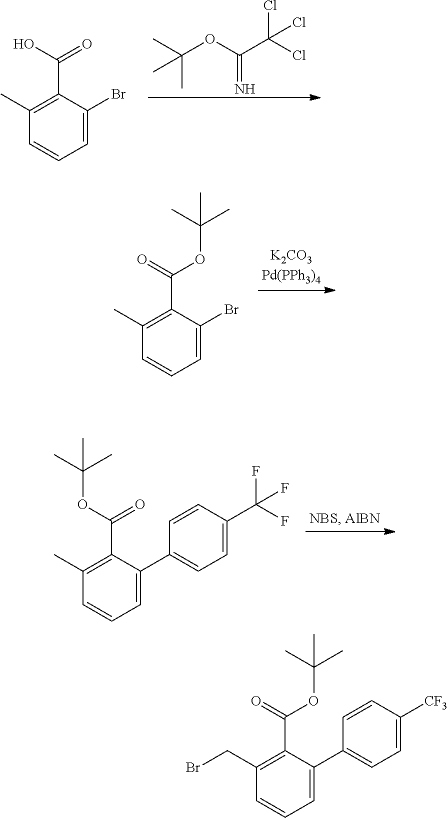

Step 1: tert-Butyl 2-bromo-6-methylbenzoate: To a solution of methyl 2-bromo-6-methylbenzoate (100 mg, 0.45 mmol) in dry THF (3 mL) was added tert-butyl 2,2,2-trichloroacetimidate (200 mg, 0.9 mmol) and boron trifluoride diethyl etherate (117 mg, 0.9 mmol). The reaction mixture was stirred at room temperature overnight and then diluted with water (50 mL) and extracted with EtOAc (80 mL×2). The combined organic layers were washed with brine, dried over Na₂SO₄, fitered, and concentrated. The residue was purified by column chromatography on silica gel (eluent: petroleum ether/EtOAc=20:1 to 15:1) to afford tert-butyl 2-bromo-6-methylbenzoate (60 mg, 24%) as a colorless liquid. ¹H NMR (400 MHz, CDCl₃) δ 7.48 (dd, J=7.1, 1.9 Hz, 1H), 7.28 (d, J=1.9 Hz, 2H), 2.29 (s, 3H), 1.56 (s, 9H).

Step 2: tert-Butyl 3-methyl-4′-(trifluoromethyl)-[1,1′-biphenyl]-2-carboxylate: To a solution of tert-butyl 2-bromo-6-methylbenzoate (200 mg, 0.74 mmol) in a mixture of dioxane and water (2 mL/0.5 mL) was added (4-(trifluoromethyl)phenyl)boronic acid (154 mg, 0.82 mmol), Pd(PPh₃)₄ (85 mg, 0.07 mmol), and K₂CO₃ (204 mg, 1.48 mmol). The mixture was stirred under a N₂ atmosphere at 100° C. for 6 hours and then diluted with water (50 mL) and extracted with EtOAc (80 mL×2). The combined organic layers were washed with brine, dried over Na₂SO₄, fitered, and concentrated. The residue was purified by column chromatography on silica gel (eluent: petroleum ether/EtOAc=20:1 to 15:1) to afford tert-butyl 3-methyl-4′-(trifluoromethyl)-[1,1′-biphenyl]-2-carboxylate (140 mg, 56%) as a colorless oil. ¹H NMR (400 MHz, CDCl₃) δ 7.67 (d, J=7.9 Hz, 2H), 7.53 (d, J=8.0 Hz, 2H), 7.37 (t, J=7.6 Hz, 1H), 7.28 (d, J=2.9 Hz, 1H), 7.17 (d, J=7.6 Hz, 1H), 2.45 (s, 3H), 1.30 (s, 9H).

Step 3: tert-Butyl 3-(bromomethyl)-4′-(trifluoromethyl)-[1,1′-biphenyl]-2-carboxylate: To a solution of tert-butyl 3-methyl-4′-(trifluoromethyl)-[1,1′-biphenyl]-2-carboxylate (140 mg, 0.42 mmol) in CCl₄ (2 mL) was added NBS (89 mg, 0.5 mmol) and AIBN (14 mg, 0.08 mmol). The reaction mixture was heated at 80° C. overnight and then diluted with water (50 mL) and extracted with EtOAc (80 mL×2). The combined organic layers were washed with brine, dried over Na₂SO₄, fitered, and concentrated. The residue obtained was purified by column chromatography on silica gel (eluent: petroleum ether/EtOAc=40/1 to 20/1) to afford methyl 3-(bromomethyl)-4′-(trifluoromethyl)-[1,1′-biphenyl]-2-carboxylate (120 mg, 69%) as a white solid. ¹H NMR (400 MHz, CDCl₃) δ 7.67 (d, J=8.0 Hz, 2H), 7.52-7.44 (m, 4H), 7.30-7.27 (m, 1H), 4.69 (s, 2H), 1.27 (s, 9H).

### Synthesis of 2-((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methyl)-3-(6-(4-(trifluoromethyl)phenyl)pyridin-2-yl)propanoic acid I-50

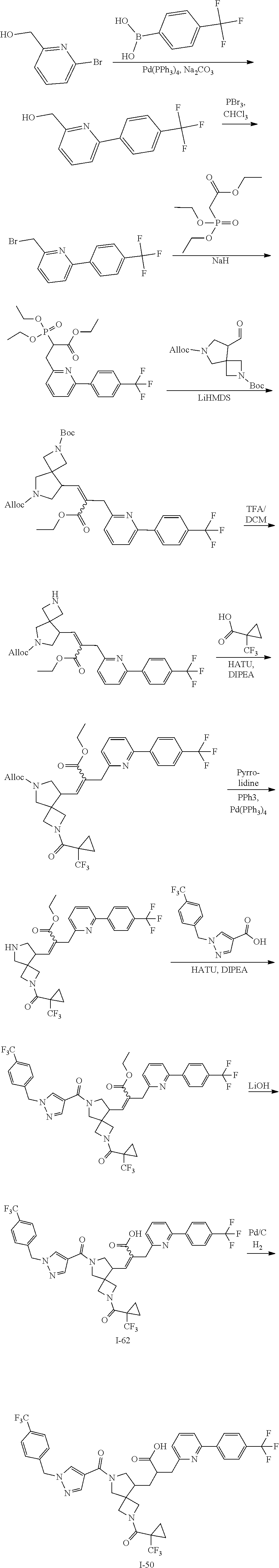

Step 1: 6-(4-(Trifluoromethyl)phenyl)pyridin-2-yl)methanol: To a solution of (6-bromopyridin-2-yl)methanol (1 g, 5.32 mmol) in a mixture of DCE and water (6 mL/3 mL) was added (4-(trifluoromethyl)phenyl)boronic acid (1 g, 5.32 mmol), Pd(PPh₃)₄ (297 mg, 0.26 mmol), and Na₂CO₃ (1.7 g, 15.96 mol). The reaction was heated under a N₂ atmosphere at 90° C. overnight and then diluted with water (100 mL) and extracted with EtOAc (100 mL×3). The combined organic layers were washed with brine, dried over Na₂SO₄, fitered, and concentreated. The residue was purified by column chromatography on silica gel (eluent: petroleum ether/EtOAc=3/1) to afford (6-(4-(trifluoromethyl)phenyl)pyridin-2-yl)methanol (1 g, 74%) as a yellow oil. ¹H NMR (400 MHz, DMSO-d₆): δ 8.30 (d, J=8.0 Hz, 2H), 7.97-7.90 (m, 2H), 7.84 (d, J=8.2 Hz, 2H), 7.52 (dd, J=6.8, 1.8 Hz, 1H), 5.49 (t, J=5.8 Hz, 1H), 4.66 (d, J=5.8 Hz, 2H).

Step 2: 2-(bromomethyl)-6-(4-(trifluoromethyl)phenyl)pyridine: To a solution of (6-(4-(trifluoromethyl)phenyl)pyridin-2-yl)methanol (5 g, 19.76 mmol) in CHCl₃ (50 mL) at 0° C. was added PBr₃ (5 mL). The reaction was stirred for 10 minutes. The reaction was allowed to warm to room temperature and stirred for another 4 hours and then diluted with water (150 mL) and extracted with DCM (150 mL×3). The combined organic layers were washed with brine, dried over Na₂SO₄, fitered, and concentreated to afford 2-(bromomethyl)-6-(4-(trifluoromethyl)phenyl)pyridine (6 g, 96%) as a white solid. ¹H NMR (400 MHz, DMSO-d₆): δ 8.30 (d, J=8.0 Hz, 2H), 8.01 (m, 2H), 7.85 (d, J=8.2 Hz, 2H), 7.59 (d, J=7.4 Hz, 1H), 4.87 (s, 2H).

Step 3: Ethyl 2-(diethoxyphosphoryl)-3-(6-(4-(trifluoromethyl)phenyl)pyridin-2-yl) propanoate: To a solution of 2-(bromomethyl)-6-(4-(trifluoromethyl)phenyl)pyridine (1 g, 3.17 mmol) in DMF (2 mL) at 0° C. was added NaH (60% in mineral oil, 254 mg, 6.34 mmol). The reaction was stirred for 10 minutes. The reaction was allowed to warm to room temperature and stirred for another 1 hour. The reaction mixture was then diluted with water (10 mL) and extracted with EtOAc (10 mL×3). The combined organic layers were washed with brine, dried over Na₂SO₄, fitered, and concentrated. The residue was purified by RP-column (eluent: MeCN/H₂O=75%:25%) to afford ethyl 2-(diethoxyphosphoryl)-3-(6-(4-(trifluoromethyl)phenyl)pyridin-2-yl)propanoate (600 mg, 41%) as a brown oil. ¹H NMR (400 MHz, DMSO-d₆): δ 8.28 (d, J=8.0 Hz, 2H), 7.93-7.83 (m, 4H), 7.38 (dd, J=7.4, 1.0 Hz, 1H), 4.13-4.01 (m, 6H), 3.79 (m, 1H), 3.48 (m, 1H), 3.31-3.24 (m, 1H), 1.26 (td, J=7.0, 4.0 Hz, 6H), 1.07 (t, J=7.0 Hz, 3H).

Step 4: 6-Allyl 2-(tert-butyl) (Z)-8-(3-ethoxy-3-oxo-2-((6-(4-(trifluoromethyl) phenyl)pyridin-2-yl)methyl)prop-1-en-1-yl)-2,6-diazaspiro[3.4]octane-2,6-dicarboxylate: To a solution of 6-allyl 2-(tert-butyl) (Z)-8-(3-ethoxy-3-oxo-2-((6-(4-(trifluoromethyl)phenyl) pyridin-2-yl)methyl)prop-1-en-1-yl)-2,6-diazaspiro[3.4]octane-2,6-dicarboxylate (500 mg, 1.54 mol) and ethyl 2-(diethoxyphosphoryl)-3-(6-(4-(trifluoromethyl)phenyl)pyridin-2-yl)propanoate (708 mg, 1.54 mol) in anhydrous THE (12 mL) at 0° C. under a N₂ atmosphere was added LiHMDS (1 M in THF, 2.3 mL, 2.31 mmol). The reaction was allowed to warm to room temperature and stirred for another 2 hours and then diluted with water (30 mL) and extracted with EtOAc (30 mL×3). The combined organic layers were washed with brine, dried over Na₂SO₄, filtered, and concentrated. The residue obtained was purified by prep-TLC (eluent: DCM/MeOH=20:1) to afford 6-allyl 2-(tert-butyl) (Z)-8-(3-ethoxy-3-oxo-2-((6-(4-(trifluoromethyl)phenyl)pyridin-2-yl)methyl)prop-1-en-1-yl)-2,6-diazaspiro[3.4]octane-2,6-dicarboxylate (500 mg, 52%) as a colorless oil. LCMS m/z=630.3 [M+H]⁺. ¹H NMR (400 MHz, DMSO-d₆): δ 8.27 (dd, J=15.6, 8.0 Hz, 2H), 7.92-7.79 (m, 4H), 7.30 (m, 1H), 6.79 (d, J=10.4 Hz, 1H), 5.96-5.79 (m, 1H), 5.35-5.06 (m, 2H), 4.53-4.42 (m, 2H), 4.04 (m, 4H), 3.84-3.50 (m, 8H), 3.23 (d, J=6.6 Hz, 1H), 1.32-1.27 (m, 9H), 1.11 (t, J=7.2 Hz, 3H).

Step 5: Allyl (Z)-8-(3-ethoxy-3-oxo-2-((6-(4-(trifluoromethyl)phenyl)pyridin-2-yl)methyl)prop-1-en-1-yl)-2,6-diazaspiro[3.4]octane-6-carboxylate: To a solution of 6-allyl 2-(tert-butyl) (Z)-8-(3-ethoxy-3-oxo-2-((6-(4-(trifluoromethyl)phenyl)pyridin-2-yl)methyl)prop-1-en-1-yl)-2,6-diazaspiro[3.4]octane-2,6-dicarboxylate (500 mg, 0.79 mmol) in DCM (5 mL) was added TFA (2 mL). The reaction was stirred at room temperature for 2 hours. The solvent was removed under vacuum to afford crude allyl (Z)-8-(3-ethoxy-3-oxo-2-((6-(4-(trifluoromethyl) phenyl)202yridine-2-yl)methyl)prop-1-en-1-yl)-2,6-diazaspiro[3.4]octane-6-carboxylate (420 mg, 100%) as a brown oil, which was used directly in the next step. LCMS m/z=530.20 [M+H]⁺.

Step 6: Allyl (Z)-8-(3-ethoxy-3-oxo-2-((6-(4-(trifluoromethyl)phenyl)pyridin-2-yl)methyl)prop-1-en-1-yl)-2-(1-(trifluoromethyl)cyclopropane-1-carbonyl)-2,6-diazaspiro [3,4]octane-6-carboxylate: To a solution of 1-(trifluoromethyl)cyclopropane-1-carboxylic acid (51 mg, 0.33 mmol) in DCM (3 mL) was added HATU (126 mg, 0.33 mmol) and DIPEA (128 mg, 0.99 mmol). The mixture was stirred at room temperature for 20 minutes. Allyl (Z)-8-(3-ethoxy-3-oxo-2-((6-(4-(trifluoromethyl)phenyl)pyridin-2-yl)methyl)prop-1-en-1-yl)-2,6-diazaspiro[3.4]octane-6-carboxylate (175 mg, 0.33 mmol) was added and stirring was continued for 2 hours. The mixture was diluted with water (30 mL) and extracted with DCM (30 mL×2). The combined organic layers were washed with brine, dried overNa₂SO₄, filtered, and concentrated. The residue obtained was purified by prep-TLC (eluent: DCM/MeOH=20:1) to afford allyl (Z)-8-(3-ethoxy-3-oxo-2-((6-(4-(trifluoromethyl)phenyl)pyridin-2-yl)methyl)prop-1-en-1-yl)-2-(1-(trifluoromethyl)cyclopropane-1-carbonyl)-2,6-diazaspiro[3.4]octane-6-carboxylate (165 mg, 75%) as a colorless oil. LCMS m/z=666.1 [M+H]⁻. ¹H NMR (400 MHz, DMSO-d₆): δ 8.26 (dd, J=12.6, 8.2 Hz, 2H), 7.93-7.75 (m, 4H), 7.29 (m, 1H), 6.81 (s, 1H), 5.99-5.77 (m, 1H), 5.19 (m, 2H), 4.57-4.42 (m, 2H), 4.20-4.02 (m, 4H), 3.89-3.36 (m, 9H), 1.47 (m, 1H), 1.36-1.27 (m, 1H), 1.12-1.08 (m, 2H), 1.01 (t, J=7.0 Hz, 3H).

Step 7: Ethyl (Z)-3-(2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate: To a solution of allyl (Z)-8-(3-ethoxy-3-oxo-2-((6-(4-(trifluoromethyl)phenyl)pyridin-2-yl)methyl)prop-1-en-1-yl)-2-(1-(trifluoromethyl)cyclopropane-1-carbonyl)-2,6-diazaspiro[3.4]octane-6-carboxylate (80 mg, 0.12 mmol) in DCM (2 mL) at 0° C. was added PPh₃ (8.0 mg, 0.03 mmol) and Pd(PPh₃)₄ (14 mg, 0.01 mmol). The mixture was stirred for 20 minutes. Pyrrolidine (10 mg, 0.14 mmol) was added. The reaction was allowed to warm to room temperature and stirred for another 1 hour. The mixture was diluted with water (5 mL) and extracted with DCM (5 mL×3). The combined organic layers were washed with brine, dried over Na₂SO₄, filtered, and concentrated to afford ethyl (Z)-3-(2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate (69 mg, 100%) as a colorless oil which was used directly in the next step. LCMS m/z=581.9 [M+H]⁺.

Step 8: Ethyl (Z)-3-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate: To a solution of 1-(4-(trifluoromethyl) benzyl)-1H-pyrazole-4-carboxylic acid (67 mg, 0.25 mmol) in DCM (3 mL) was added HATU (94 mg, 0.25 mmol) and DIPEA (96 mg, 0.75 mmol). The mixture was stirred for 20 minutes. Ethyl (Z)-3-(2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate (144 mg, 0.25 mmol) was added and stirring was continued for another 2 hours. The mixture was diluted with water (30 mL) and extracted with DCM (30 mL×2). The combined organic layers were washed with brine, dried over Na₂SO₄, filtered, and concentrated. The residue obtained was purified by prep-TLC (eluent: DCM/MeOH=20:1) to afford ethyl (Z)-3-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl) acrylate (150 mg, 73%) as a colorless oil. LCMS m/z=834.3 [M+H]⁺. ¹H NMR (400 MHz, DMSO-d₆): δ 8.44-8.31 (m, 1H), 8.29-8.17 (m, 1H), 7.97-7.51 (m, 8H), 7.48-7.25 (m, 3H), 6.84-6.8 (m, 1H), 5.47 (s, 2H), 4.09-3.35 (m, 12H), 3.13 (m, 1H), 1.25 (d, J=2.8 Hz, 3H), 1.10 (q, J=6.8 Hz, 2H), 1.02 (t, J=7.4 Hz, 2H).

Step 9: 3-(6-(1-(4-(Trifluoromethyl)benzyl)-1H-pyrazole-4-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylic acid I-62: To a solution of ethyl (Z)-3-(6-(1-(4-(trifluoromethyl)benzyl)-1H-pyrazole-4-carbonyl)-2-(1-(trifluoromethyl) cyclopropane-1-carbonyl)-2,6-diazaspiro[3.4]octan-8-yl)-2-((6-(4-(trifluoromethyl)phenyl) pyridine-2-yl)methyl)acrylate (150 mg, 0.18 mmol) in a mixture of THF, water, and MeOH (1.5 mL/0.5 mL/0.5 mL) was added LiOH (23 mg, 0.54 mmol). The reaction was stirred at room temperature for 2 hours. The mixture was diluted with water (15 mL) and extracted with EtOAc (15 mL×2). The aqueous layer was collected, acidified to pH˜2 with 1 M HCl, and extracted with EtOAc (20 mL×2). The combined organic layers were washed with brine, dried over Na₂SO₄, filtered, and concentrated to afford 3-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylic acid (52 mg, 36%) as a white solid. LCMS m/z=806.1 [M+H]⁺. ¹H NMR (400 MHz, DMSO-d₆): δ 8.45-8.41 (m, 1H), 8.32-8.20 (m, 2H), 7.91-7.70 (m, 7H), 7.42 (m, 2H), 7.28 (dd, J=18.8, 6.8 Hz, 1H), 6.84-6.8 (m, 1H), 5.51-5.41 (m, 2H), 4.08-3.64 (m, 10H), 3.51-3.35 (m, 1H), 1.04 (d, J=11.4 Hz, 4H).

Step 10: 2-((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methyl)-3-(6-(4-(trifluoromethyl)phenyl)pyridin-2-yl)propanoic acid I-50: To a solution of 3-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl) acrylic acid (140 mg, 0.17 mmol) in MeOH (5 mL) was added 10% Pd/C (56 mg). The reaction was heated at 35° C. under a H₂ atmosphere overnight. The catalyst was removed by filtration through Celite, and the filtrate was concentrated. The residue obtained was purified by prep-HPLC to afford 2-((6-(1-(4-(trifluoromethyl)benzyl)-1H-pyrazole-4-carbonyl)-2-(1-(trifluoromethyl) cyclopropane-1-carbonyl)-2,6-diazaspiro[3.4]octan-8-yl)methyl)-3-(6-(4-(trifluoromethyl) phenyl)pyridin-2-yl)propanoic acid (62 mg, 44%) as a white solid. LCMS m/z=808.1 [M+H]⁺. ¹H NMR (400 MHz, DMSO-d₆): δ 8.42-8.25 (m, 3H), 7.93-7.77 (m, 5H), 7.72 (d, J=8.6 Hz, 2H), 7.43 (d, J=8.8 Hz, 2H), 7.35-7.26 (m, 1H), 5.47 (t, J=7.6 Hz, 2H), 4.08-3.38 (m, 8H), 3.17 (m, 2H), 3.02 (m, 1H), 1.98-1.50 (m, 3H), 1.12 (m, 4H).

### Synthesis of 3-(6-(5-hydroxypyrazine-2-carbonyl)-2-(1-(trifluoromethyl) cyclopropane-1-carbonyl)-2,6-diazaspiro[3.4]octan-8-yl)-2-((6-(4-(trifluoromethyl)phenyl) pyridin-2-yl)methyl)acrylic acid I-63

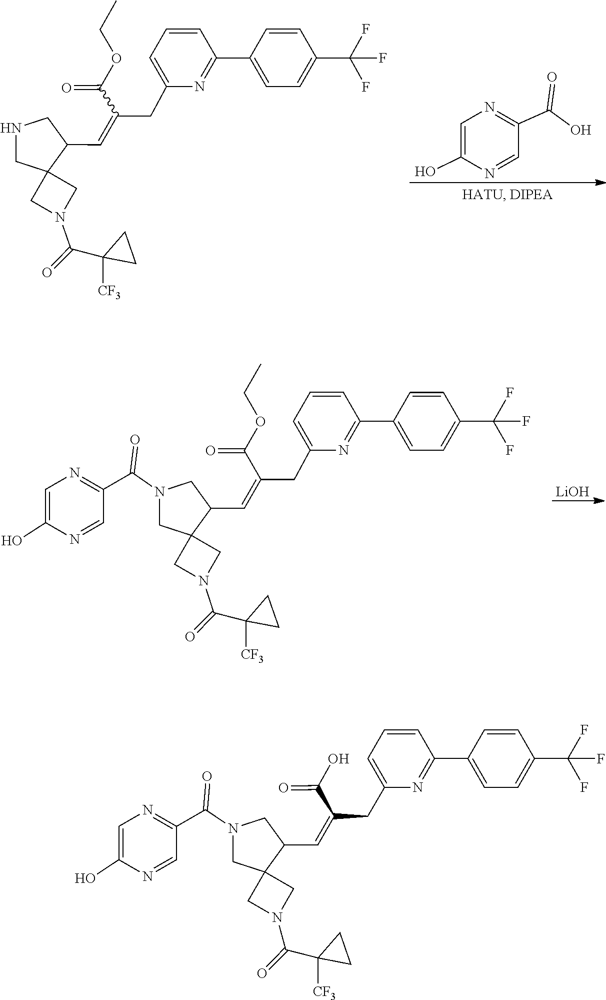

Step 1: ethyl 3-(6-(5-hydroxypyrazine-2-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate: To a solution of 5-hydroxypyrazine-2-carboxylic acid (12 mg, 0.09 mmol) in DCM (2 mL) was added HATU (33 mg, 0.09 mmol) and DIPEA (33 mg, 0.26 mmol). The mixture was stirred at room temperature for 30 minutes. Ethyl 3-(2-(1-(trifluoromethyl) cyclopropane-1-carbonyl)-2,6-diazaspiro[3.4]octan-8-yl)-2-((6-(4-(trifluoromethyl)phenyl) pyridin-2-yl)methyl)acrylate (50 mg, 0.09 mmol) was added, and stirring was continued for 2 hours. The mixture was diluted with water (10 mL) and extracted with DCM (10 mL×2). The combined organic layers were washed with brine, dried over Na₂SO₄, filtered, and concentrated. The residue obtained was purified by RP-Column (eluent: MeCN/H₂O=65%:35%) to afford ethyl 3-(6-(5-hydroxypyrazine-2-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate (40 mg, 66%) as a colorless oil. LCMS m/z=704.2 [M+H]⁺. ¹H NMR (400 MHz, DMSO-d₆): δ 8.23 (m, 2H), 8.01-7.69 (m, 6H), 7.35-7.24 (m, 1H), 6.83 (s, 1H), 4.13-3.66 (m, 12H), 3.51-3.40 (m, 1H), 1.10 (td, J=7.0, 3.2 Hz, 3H), 1.01 (d, J=8.6 Hz, 2H), 0.85 (t, J=6.6 Hz, 2H).

Step 2: 3-(6-(5-hydroxypyrazine-2-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylic acid I-63: To a solution of ethyl 3-(6-(5-hydroxypyrazine-2-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylate (40 mg, 0.06 mmol) in a mixture of THF, MeOH, and water (1 mL/0.3 mL/0.3 mL) was added LiGH (7 mg, 0.17 mmol), and the mixture was stirred at room temperature for 2 hours. The reaction was diluted with water (10 mL) and extracted with EtOAc (10 mL). The aqueous layer was collected, acidified with 1 M HCl to pH˜2, and extracted with EtOAc (15 mL×2). The combined organic layers were washed with brine, dried over Na₂SO₄, filtered, and concentrated. The residue obtained was purified by prep-HPLC to afford 3-(6-(5-hydroxypyrazine-2-carbonyl)-2-(1-(trifluoromethyl)cyclopropane-1-carbonyl)-2,6-diazaspiro[3.4]octan-8-yl)-2-((6-(4-(trifluoromethyl)phenyl)pyridin-2-yl)methyl)acrylic acid (15 mg, 39%) as a white solid. LCMS m/z=676.1 [M+H]⁺. ¹H NMR (400 MHz, DMSO-d6) δ 12.54 (s, 1H), 8.25 (dd, J=22.0, 8.0 Hz, 2H), 7.98-7.69 (m, 6H), 7.28 (m, 1H), 4.15-3.63 (m, 10H), 3.48 (s, 1H), 1.21-1.00 (m, 4H).

### Synthesis of (2-((R)-2,2-difluorocyclopropane-1-carbonyl)-8-((((6-(4-(trifluoromethyl)phenyl)pyridin-2-yl)methyl)sulfonyl)methyl)-2,6-diazaspiro[3.4]octan-6-yl)(5-hydroxypyrazin-2-yl)methanone I-3

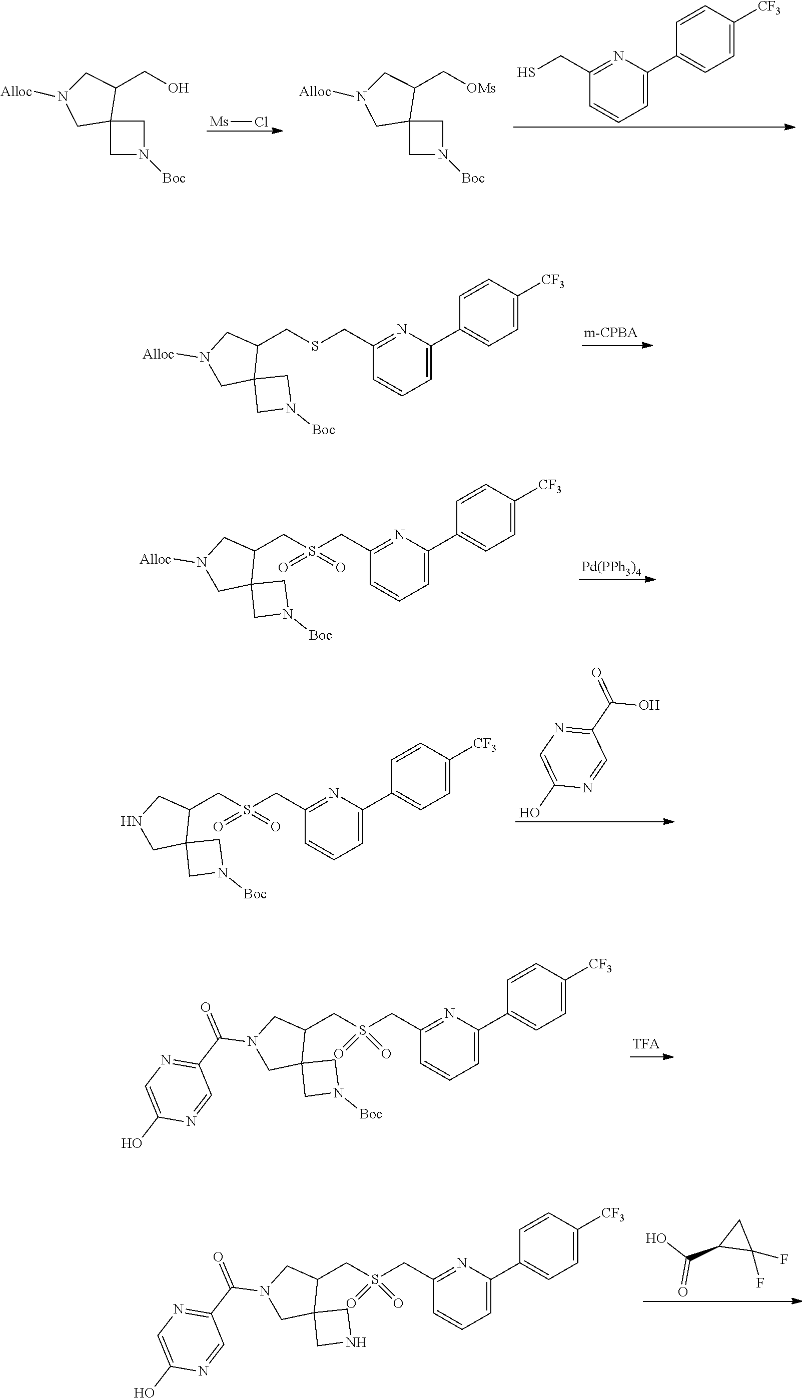

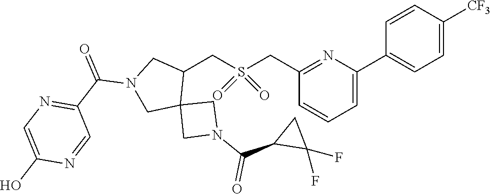 [SMILES: O=C(c1cnc(O)cn1)N1CC(CS(=O)(=O)Cc2cccc(-c3ccc(C(F)(F)F)cc3)n2)C2(C1)CN(C(=O)[C@H]1CC1(F)F)C2]

Step 1: 6-allyl 2-(tert-butyl) 8-(((methylsulfonyl)oxy)methyl)-2,6-diazaspiro[3.4]octane-2,6-dicarboxylate: To a solution of 6-allyl 2-(tert-butyl) 8-(hydroxymethyl)-2,6-diazaspiro[3.4]octane-2,6-dicarboxylate (700 mg, 2.14 mmol) in DCM (8 mL) at 0° C. was added TEA (0.5 mL, 4.28 mmol) and MsCl (0.26 mL, 3.21 mmol). The reaction was allowed to warm to room temperature, stirred for 1 hour, and then diluted with water (10 mL) and extracted with DCM (20 mL×2). The combined organic layers were washed with brine, dried over Na₂SO₄, filtered, and concentrated under reduced pressure to afford 6-allyl 2-(tert-butyl) 8-(((methylsulfonyl)oxy)methyl)-2,6-diazaspiro[3.4]octane-2,6-dicarboxylate (1.1 g, 81%) as a yellow oil. LCMS m/z=405.2 [M+H]⁺. ¹H NMR (400 MHz, DMSO-d₆): δ 6.02-5.85 (m, 1H), 5.28 (dd, J=17.4, 6.6 Hz, 1H), 5.18 (d, J=12.3 Hz, 1H), 4.58-4.47 (m, 2H), 4.46-4.38 (m, 1H), 4.31-4.19 (m, 1H), 4.03 (d, J=9.1 Hz, 1H), 3.87-3.64 (m, 3H), 3.59-3.37 (m, 4H), 3.21 (s, 3H), 2.70-2.58 (m, 1H), 1.38 (s, 9H).

Step 2: 6-allyl 2-(tert-butyl) 8-((((6-(4-(trifluoromethyl)phenyl)pyridin-2-yl)methyl) thio)methyl)-2,6-diazaspiro[3.4]octane-2,6-dicarboxylate: To a solution of 6-allyl 2-(tert-butyl) 8-(((methylsulfonyl)oxy)methyl)-2,6-diazaspiro[3.4]octane-2,6-dicarboxylate (50 mg, 0.12 mmol) in acetonitrile (1.0 mL) at room temperature was added cesium carbonate (78 mg, 0.24 mmol) and 6-(4-(trifluoromethyl)phenyl)pyridine-2-thiol (33 mg, 0.12 mmol). The reaction mixture was heated at reflux overnight. Next, the reaction was filtered through Celite, and the filtrate was concentrated under reduced pressure. The residue obtained was purified by prep-TLC (DCM/MeOH=30:1) to afford 8-((((6-(4-(trifluoromethyl)phenyl)pyridin-2-yl)methyl)thio) methyl)-2,6-diazaspiro[3.4]octane-2,6-dicarboxylate (40 mg, 56%) as a colorless solid. LCMS m/z=578.2 [M+H]⁺. ¹H NMR (400 MHz, CD₃OD): δ 8.22 (d, J=8.2 Hz, 2H), 7.92-7.81 (m, 2H), 7.78 (d, J=8.2 Hz, 2H), 7.49 (d, J=7.4 Hz, 1H), 5.98-5.86 (m, 1H), 5.32-5.15 (m, 2H), 4.57-4.49 (m, 2H), 4.03-3.91 (m, 3H), 3.81-3.71 (m, 2H), 3.65-3.48 (m, 4H), 3.29-3.25 (m, 1H), 2.91-2.79 (m, 1H), 2.53-2.40 (m, 2H), 1.38 (s, 9H).

Step 3: 6-allyl 2-(tert-butyl) 8-((((6-(4-(trifluoromethyl)phenyl)pyridin-2-yl)methyl) sulfonyl)methyl)-2,6-diazaspiro[3.4]octane-2,6-dicarboxylate: To a solution of 8-((((6-(4-(trifluoromethyl)phenyl)pyridin-2-yl)methyl)thio)methyl)-2,6-diazaspiro[3.4]octane-2,6-dicarboxylate (18 mg, 0.031 mmol) in DCM (1.0 mL) was added m-CPBA (80% purity, 15 mg, 0.069 mmol). The reaction was stirred at room temperature for 10 minutes. The mixture was diluted with water (10 mL) and extracted with DCM (20 mL×3). The combined organic phases were washed with brine, dried over Na₂SO₄, filtered, and concentrated under reduced pressure to afford 2-(tert-butyl) 8-((((6-(4-(trifluoromethyl)phenyl)pyridin-2-yl)methyl)sulfonyl)methyl)-2,6-diazaspiro[3.4]octane-2,6-dicarboxylate (13 mg, 68%) as a yellow solid. LCMS m/z=610.1 [M+H]⁺. ¹H NMR (400 MHz, DMSO-d₆): δ 7.46 (d, J=8.2 Hz, 2H), 7.24-7.14 (m, 2H), 7.02 (d, J=8.2 Hz, 2H), 6.81-6.75 (m, 1H), 5.22-5.02 (m, 1H), 4.55-4.36 (m, 2H), 4.02-3.94 (m, 1H), 3.79-3.71 (m, 2H), 3.15-2.62 (m, 10H), 2.60-2.54 (m, 1H), 2.14-2.02 (m, 1H), 0.59 (s, 9H).

Step 4: tert-butyl 8-((((6-(4-(trifluoromethyl)phenyl)pyridin-2-yl)methyl)sulfonyl) methyl)-2,6-diazaspiro[3.4]octane-2-carboxylate: To a solution of 6-allyl 2-(tert-butyl) 8-((((6-(4-(trifluoromethyl)phenyl)pyridin-2-yl)methyl)sulfonyl)methyl)-2,6-diazaspiro[3.4]octane-2,6-dicarboxylate (130 mg, 0.21 mmol) in THF (3 mL) was added phenylsilane (113 mg, 1.05 mmol) and Pd(PPh₃)₄ (25 mg, 0.02 mmol). The reaction was stirred at room temperature for 20 minutes. The mixture was diluted with water (20 mL) and extracted with EtOAc (30 mL×2). The combined organic layers were washed with brine, dried over Na₂SO₄, filtered, and concentrated. The residue was purified by column chromatography on silica gel (DCM/MeOH=30:1) to afford tert-butyl 8-((((6-(4-(trifluoromethyl)phenyl)pyridin-2-yl)methyl)sulfonyl)methyl)-2,6-diazaspiro[3.4]octane-2-carboxylate (40 mg, 36%) as a brown solid. LCMS m/z=526.2 [M+H]⁺. ¹H NMR (400 MHz, Chloroform-d) δ 8.09 (d, J=8.1 Hz, 2H), 7.89 (t, J=7.8 Hz, 1H), 7.84-7.72 (m, 3H), 7.53-7.48 (m, 1H), 4.65-4.46 (m, 2H), 3.82-3.60 (m, 4H), 3.50-3.38 (m, 2H), 3.27-3.07 (m, 3H), 2.92-2.85 (m, 1H), 2.74-2.64 (m, 1H), 1.39 (s, 9H).

Step 5: tert-butyl 6-(5-hydroxypyrazine-2-carbonyl)-8-((((6-(4-(trifluoromethyl) phenyl)pyridine-2-yl)methyl)sulfonyl)methyl)-2,6-diazaspiro[3.4]octane-2-carboxylate: To a solution of solution of 5-hydroxypyrazine-2-carboxylic acid (13 mg, 0.091 mmol) in DMF (1.0 mL) was added HATU (35 mg, 0.091 mmol) and DIPEA (29 mg, 0.23 mmol). The mixture was stirred at room temperature for 30 minutes. tert-Butyl 8-((((6-(4-(trifluoromethyl)phenyl)pyridin-2-yl)methyl)sulfonyl)methyl)-2,6-diazaspiro[3.4]octane-2-carboxylate (40 mg, 0.076 mmol) was added and stirring was continued for 2 hours. The solvent was removed, and the residue obtained was purified by RP-column (30% water in CH₃CN) to afford tert-butyl 6-(5-hydroxypyrazine-2-carbonyl)-8-((((6-(4-(trifluoromethyl)phenyl)pyridin-2-yl)methyl)sulfonyl)methyl)-2,6-diazaspiro[3.4]octane-2-carboxylate (40 mg, 81%) as a yellow solid. LCMS m/z=648.1 [M+H]⁺. ¹H NMR (400 MHz, CD₃OD): δ 8.32-8.20 (m, 2H), 8.08-7.86 (m, 4H), 7.79 (t, J=8.6 Hz, 2H), 7.63-7.51 (m, 1H), 4.81-4.71 (m, 2H), 4.29-3.47 (m, 10H), 2.97-2.86 (m, 1H), 1.39 (d, J=7.0 Hz, 9H).

Step 6: (5-hydroxypyrazin-2-yl)(8-((((6-(4-(trifluoromethyl)phenyl)pyridin-2-yl) methyl)sulfonyl)methyl)-2,6-diazaspiro[3.4]octan-6-yl)methanone: To a solution of tert-butyl 6-(5-hydroxypyrazine-2-carbonyl)-8-((((6-(4-(trifluoromethyl)phenyl)pyridin-2-yl)methyl) sulfonyl)methyl)-2,6-diazaspiro[3.4]octane-2-carboxylate (40 mg, 0.062 mmol) in DCM (1.0 mL) was added TFA (0.5 mL), and the reaction was stirred at room temperature for 2 hours. The solvent was removed under vacuum to afford (5-hydroxypyrazin-2-yl)(8-((((6-(4-(trifluoromethyl)phenyl) pyridin-2-yl)methyl)sulfonyl)methyl)-2,6-diazaspiro[3.4]octan-6-yl)methanone (34 mg, quant.) as an oil which was used directly in the next step.

Step 7: (2-((R)-2,2-difluorocyclopropane-1-carbonyl)-8-((((6-(4-(trifluoromethyl) phenyl)pyridin-2-yl)methyl)sulfonyl)methyl)-2,6-diazaspiro[3.4]octan-6-yl)(5-hydroxypyrazin-2-yl)methanone I-3: To a solution of (R)-2,2-difluorocyclopropane-1-carboxylic acid (9 mg, 0.072 mmol) in DMF (1.0 mL) was added HATU (27 mg, 0.072 mmol) and DIPEA (23 mg, 0.18 mmol). The mixture was stirred at room temperature for 30 minutes. (5-Hydroxypyrazin-2-yl)(8-((((6-(4-(trifluoromethyl)phenyl)pyridin-2-yl)methyl)sulfonyl)methyl)-2,6-diazaspiro[3.4]octan-6-yl)methanone (33 mg, 0.060 mmol) was added, and the reaction was stirred for another 2 hours. The mixture was purified by RP-column (30% water in CH₃CN=30) to afford (2-((R)-2,2-difluorocyclopropane-1-carbonyl)-8-((((6-(4-(trifluoromethyl)phenyl) pyridin-2-yl)methyl)sulfonyl)methyl)-2,6-diazaspiro[3.4]octan-6-yl)(5-hydroxypyrazin-2-yl) methanone (7 mg, 18%) as a white solid. LCMS m/z=652.1 [M+H]⁺. ¹H NMR (400 MHz, CD₃OD) δ 8.32-8.22 (m, 2H), 8.07-7.92 (m, 4H), 7.86-7.77 (m, 2H), 7.65-7.57 (m, 1H), 4.86-4.70 (m, 2H), 4.37-3.49 (m, 10H), 3.09-2.97 (m, 1H), 2.63-2.32 (m, 1H), 2.03-1.93 (m, 1H), 1.88-1.63 (m, 1H).

The compounds listed in Table 9 were synthesized according to the procedures outlined for the synthesis of I-3 using the appropriate commercially available reagents and/or intermediates described elsewhere.

TABLE 9
Additional Compounds

| Compound | | | |
|---|---|---|---|
| No. | Structure | ¹HNMR | LCMS |
| I-2 | 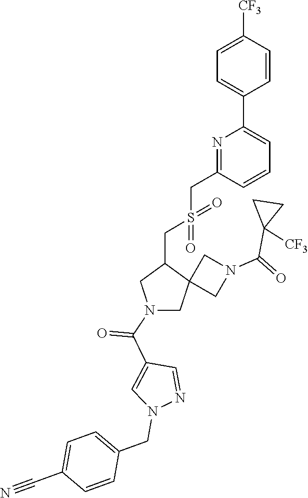 [SMILES: N#Cc1ccc(Cn2cc(C(=O)N3CC(CS(=O)(=O)Cc4cccc(-c5ccc(C(F)(F)F)cc5)n4)C4(C3)CN(C(=O)C3(C(F)(F)F)CC3)C4)cn2)cc1] | ¹H NMR (400 MHz, CD₃OD) δ 8.28-8.16 (m, 3H), 8.02-7.65 (m, 7H), 7.57 (t, J = 6.9 Hz, 1H), 7.38 (dd, J = 17.6, 7.6 Hz, 2H), 5.48-5.44 (m, 2H), 4.82-4.71 (m, 1H), 4.46- 3.40 (m, 11H), 3.15- 2.97 (m, 1H), 1.09 (s, 4H). | m/z = 771.2 [M + H]⁺ |

### Synthesis of 6-(1-benzyl-1H-pyrazole-4-carbonyl)-N-((3-cyclohexylphenyl)sulfonyl)-2-((R)-2,2-difluorocyclopropane-1-carbonyl)-2,6-diazaspiro[3.4]octane-8-carboxamide I-1

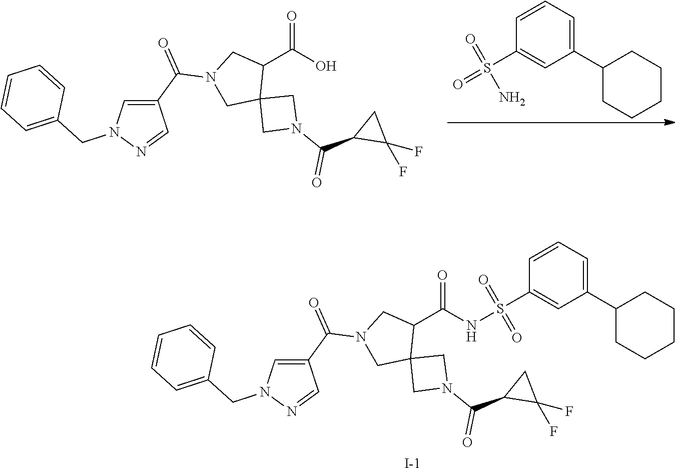 [SMILES: O=C(O)C1CN(C(=O)c2cnn(Cc3ccccc3)c2)CC12CN(C(=O)[C@H]1CC1(F)F)C2 ; NS(=O)(=O)c1cccc(C2CCCCC2)c1 ; O=C(NS(=O)(=O)c1cccc(C2CCCCC2)c1)C1CN(C(=O)c2cnn(Cc3ccccc3)c2)CC12CN(C(=O)[C@H]1CC1(F)F)C2 ; [I-]]

Step 1: 6-(1-benzyl-1H-pyrazole-4-carbonyl)-N-((3-cyclohexylphenyl)sulfonyl)-2-((R)-2,2-difluorocyclopropane-1-carbonyl)-2,6-diazaspiro[3.4]octane-8-carboxamide: To a solution of 6-(1-benzyl-1H-pyrazole-4-carbonyl)-2-((R)-2,2-difluorocyclopropane-1-carbonyl)-2,6-diazaspiro[3.4]octane-8-carboxylic acid (170 mg, 0.38 mmol) in DF (2 mL) was added HATU (174 mg, 0.46 mmol) and DIPEA (148 mg, 1.15 mmol). The mixture was stirred at room temperature for 15 minutes. 2-(3-Chloro-4-(trifluoromethyl)benzyl)oxirane (110 mg, 0.46 mmol) was added, and stirring was continued for 4 hours. The mixture was diluted with water (30 mL) and extracted with EtOAc (50 mL×3). The combined organic layers were washed with water, brine, dried over Na₂SO₄, filtered, and concentrated. The residue was purified by prep-HPLC to afford 6-(1-benzyl-1H-pyrazole-4-carbonyl)-N-((3-cyclohexylphenyl)sulfonyl)-2-((R)-2,2-difluorocyclopropane-1-carbonyl)-2,6-diazaspiro[3.4]octane-8-carboxamide (12 mg, 5%) as a white solid. LCMS m/z=666.1 [M+H]⁺. ¹H NMR (400 MHz, CD₃OD): δ 8.18 (s, 1H), 7.91-7.65 (m, 3H), 7.50-7.17 (m, 7H), 5.36 (s, 2H), 4.43-3.52 (m, 8H), 3.13 (s, 1H), 2.56 (s, 1H), 2.12-1.62 (m, 7H), 1.30 (s, 5H), 0.91 (d, J=6.1 Hz, 1H).

### Synthesis of 4-(1-(2-((6-(1-benzyl-1H-pyrazole-4-carbonyl)-2-(1-(trifluoromethyl)cyclopropane-1-carbonyl)-2,6-diazaspiro[3.4]octan-8-yl)methoxy)acetyl) piperidin-4-yl)-3-(cyclohexyloxy)benzoic acid I-60

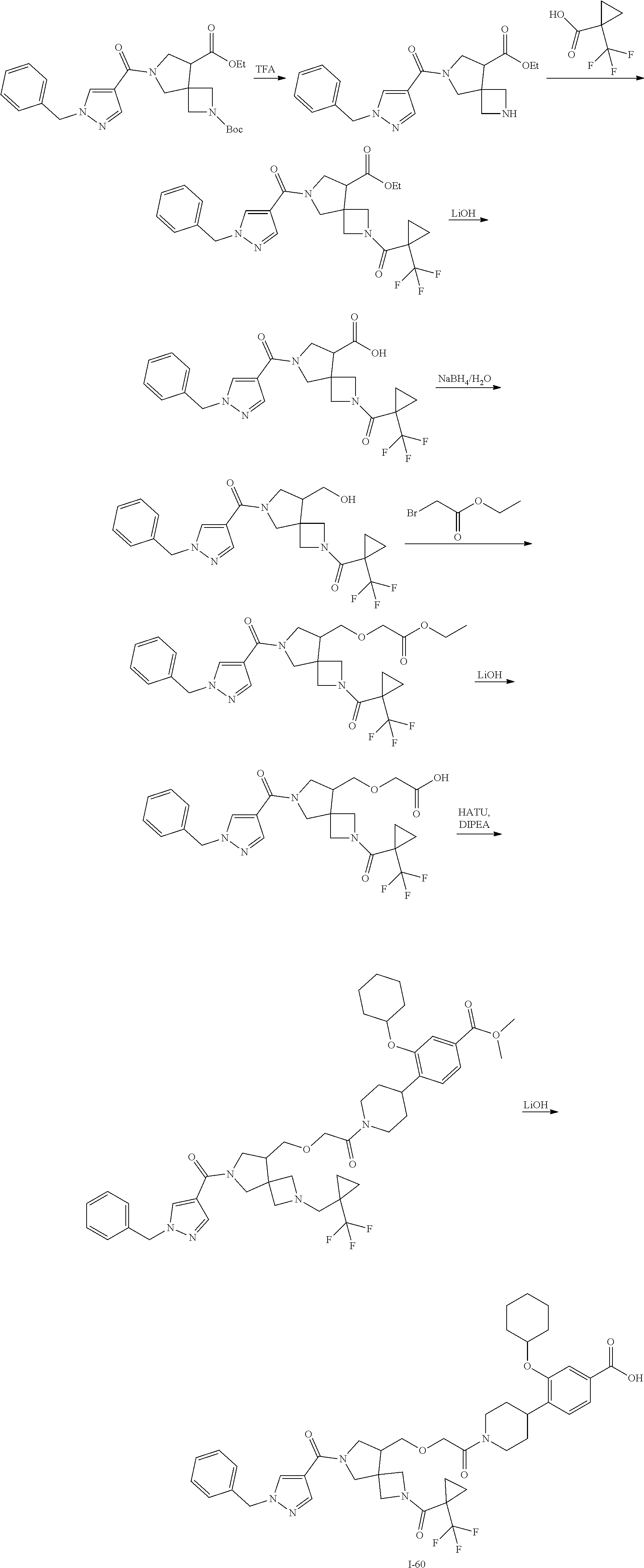

Step 1: ethyl 6-(1-benzyl-1H-pyrazole-4-carbonyl)-2,6-diazaspiro[3.4]octane-8-carboxylate: To a solution of 2-(tert-butyl) 8-ethyl 6-(1-benzyl-1H-pyrazole-4-carbonyl)-2,6-diazaspiro[3.4]octane-2,8-dicarboxylate (300 mg, 0.64 mmol) in DCM (3 mL) was added TFA (1.5 mL). The reaction mixture was stirred at room temperature for 2 hours. The solvent was removed under vacuum to afford ethyl 6-(1-benzyl-1H-pyrazole-4-carbonyl)-2,6-diazaspiro[3.4]octane-8-carboxylate (236 mg, quant.) as an oil, which was used directly in the next step. LCMS m/z=369.2 [M+H]⁻. ¹HNMR (400 MHz, CD₃OD). δ 8.19 (d, J=9.2 Hz, 1H), 7.90 (d, J=8.6 Hz, 1H), 7.38-7.24 (m, 5H), 5.38 (s, 2H), 4.44-3.74 (m, 11H), 3.55-3.43 (m, 1H), 1.32-1.27 (in, 3H).

Step 2: ethyl 6-(1-benzyl-1H-pyrazole-4-carbonyl)-2-(1-(trifluoromethyl) cyclopropane-1-carbonyl)-2,6-diazaspiro[3.4]octane-8-carboxylate: To a solution of 1-(trifluoromethyl)cyclopropane-1-carboxylic acid (1.09 g, 7.05 mmol) in DCM was added HATU (2.68 g, 7.05 mmol) and DIPEA (2.48 g, 19.23 mmol). The mixture was stirred for 15 minutes. Ethyl 6-(1-benzyl-1H-pyrazole-4-carbonyl)-2,6-diazaspiro[3.4]octane-8-carboxylate (2.36 g, 6.41 mmol) was added and stirring was continued for 2 hours. The mixture was diluted with water (100 mL) and extracted with DCM (150 mL×2). The combined organic layers were washed with brine, dried over Na₂SO₄, filtered, and concentrated. The mixture was purified by RP-column (50% water in CH₃CN) to afford ethyl 6-(1-benzyl-1H-pyrazole-4-carbonyl)-2-(1-(trifluoromethyl) cyclopropane-1-carbonyl)-2,6-diazaspiro[3.4]octane-8-carboxylate (2.6 g, 80%) as a yellow solid. LCMS m/z=505.2 [M+H]⁺. ¹HNMR (400 MHz, DMSO-d₆) δ 8.35 (d, J=8.2 Hz, 1H), 7.82 (d, J=14.6 Hz, 1H), 7.39-7.22 (m, 5H), 5.36 (s, 2H), 4.33-3.64 (m, 10H), 3.51-3.34 (m, 1H), 1.29-1.14 (m, 7H).

Step 3: 6-(1-benzyl-1H-pyrazole-4-carbonyl)-2-(1-(trifluoromethyl)cyclopropane-1-carbonyl)-2,6-diazaspiro[3.4]octane-8-carboxylic acid: To a solution of ethyl 6-(1-benzyl-1H-pyrazole-4-carbonyl)-2-(1-(trifluoromethyl)cyclopropane-1-carbonyl)-2,6-diazaspiro[3.4]octane-8-carboxylate (2.4 g, 4.76 mmol) in a mixture of EtOH and water (15 mL/3 mL) was added LiOH (400 mg, 28.54 mmol). The mixture was stirred at room temperature for 2 hours and then diluted with water (70 mL) and extracted with EtOAc (50 mL). The aqueous phase was collected, acidified with 1 M HCl to pH˜2, and extracted with EtOAc (90 mL×3). The combined organic layers were washed with brine, dried over Na₂SO₄ filtered, and concentrated to afford 6-(1-benzyl-1H-pyrazole-4-carbonyl)-2-(1-(trifluoromethyl)cyclopropane-1-carbonyl)-2,6-diazaspiro[3.4]octane-8-carboxylic acid (2.2 g, quant) as a white solid. LCMS m/z=477.1 [M+H]⁺. ¹HNMR (400 MHz, CD₃OD): δ 8.21 (d, J=8.6 Hz, 1H), 7.92 (d, J=6.6 Hz, 1H), 7.38-7.24 (m, 5H), 5.38 (s, 2H), 4.65-4.29 (m, 2H), 4.17-3.78 (m, 6H), 3.43-3.33 (m, 1H), 1.25-1.21 (m, 4H).

Step 4: (1-benzyl-1H-pyrazol-4-yl)(8-(hydroxymethyl)-2-(1-(trifluoromethyl) cyclopropane-1-carbonyl)-2,6-diazaspiro[3.4]octan-6-yl)methanone: To a solution of 6-(1-benzyl-1H-pyrazole-4-carbonyl)-2-(1-(trifluoromethyl)cyclopropane-1-carbonyl)-2,6-diazaspiro [3.4]octane-8-carboxylic acid (160 mg, 0.34 mmol) in THF (4.0 mL) was added 4-methylmorpholine (46 mg, 0.45 mmol) and isobutyl chloroformate (66 mg, 0.48 mmol) at 0° C. After stirring 30 minutes, NaBH₄ (38 mg, 1.01 mmol) in water (0.5 mL) was added dropwise. The mixture was stirred at room temperature for 3 hours. The mixture was quenched with H₂O (30 mL) and extracted with EtOAc (40 mL×3). The combined organic layers were washed with brine, dried over Na₂SO₄, filtered, and concentrated to afford (1-benzyl-1H-pyrazol-4-yl)(8-(hydroxymethyl)-2-(1-(trifluoromethyl)cyclopropane-1-carbonyl)-2,6-diazaspiro[3.4]octan-6-yl)methanone (130 mg, 82%) as a yellow oil. LCMS m/z=463.2 [M+H]⁺. ¹HNMR (400 MHz, DMSO-d₆): δ 8.32 (d, J=4.5 Hz, 1H), 7.82 (d, J=10.7 Hz, 1H), 7.38-7.23 (m, 5H), 5.35 (s, 2H), 4.79 (s, 1H), 4.30-3.40 (m, 9H), 2.86-2.71 (m, 2H), 1.25-1.15 (m, 4H).

Step 5: ethyl 2-((6-(1-benzyl-1H-pyrazole-4-carbonyl)-2-(1-(trifluoromethyl) cyclopropane-1-carbonyl)-2,6-diazaspiro[3.4]octan-8-yl)methoxy)acetate: To a solution of (1-benzyl-1H-pyrazol-4-yl)(8-(hydroxymethyl)-2-(1-(trifluoromethyl)cyclopropane-1-carbonyl)-2,6-diazaspiro[3.4]octan-6-yl)methanone (130 mg, 0.28 mmol) in THF (2 mL) at 0° C. was added KI (5.0 mg, 0.03 mmol) and NaH (22 mg, 0.56 mmol). The reaction was stirred for 30 minutes. Ethyl 2-bromoacetate (141 mg, 0.84 mmol) was added, and the reaction was allowed to warm to room temperature and stirred for another 2 hours. The mixture was diluted with water (30 mL) and extracted with EtOAc (30 mL×2). The combined organic layers were washed with brine, dried over Na₂SO₄, filtered, and concentrated. The crude product was purified by prep-TLC (eluent: DCM/MeOH=90:7) to afford ethyl 2-((6-(1-benzyl-1H-pyrazole-4-carbonyl)-2-(1-(trifluoromethyl)cyclopropane-1-carbonyl)-2,6-diazaspiro[3.4]octan-8-yl)methoxy)acetate (30 mg, 20%) as a yellow solid. LCMS m/z=549.2 [M+H]⁺. ¹HNMR (400 MHz, CD₃OD): δ 8.20 (d, J=9.8 Hz, 1H), 7.91 (d, J=6.6 Hz, 1H), 7.38-7.24 (m, 5H), 5.37 (s, 2H), 4.68 (d, J=12.4 Hz, 2H), 4.29-3.63 (m, 12H), 2.74-2.59 (m, 1H), 1.30-1.25 (m, 3H), 1.25-1.21 (m, 4H).

Step 6: 2-((6-(1-benzyl-1H-pyrazole-4-carbonyl)-2-(1-(trifluoromethyl)cyclopropane-1-carbonyl)-2,6-diazaspiro[3.4]octan-8-yl)methoxy)acetic acid: To a solution of ethyl 2-((6-(1-benzyl-1H-pyrazole-4-carbonyl)-2-(1-(trifluoromethyl)cyclopropane-1-carbonyl)-2,6-diazaspiro[3.4]octan-8-yl)methoxy)acetate (30 mg, 0.05 mmol) in a mixture of MeOH and water (0.5 mL/0.2 mL) was added LiOH (5 mg, 0.11 mmol). The mixture was stirred at room temperature for 2 hours. The reaction was diluted with water (20 mL) and extracted with EtOAc (20 mL). The aqueous phase was collected, acidified with 1 M HCl to pH˜2, and extracted with EtOAc (30 mL 3). The combined organic layers were washed with brine, dried over Na₂SO₄, and concentrated to afford 2-((6-(1-benzyl-1H-pyrazole-4-carbonyl)-2-(1-(trifluoromethyl) cyclopropane-1-carbonyl)-2,6-diazaspiro[3.4]octan-8-yl)methoxy)acetic acid (20 mg, 80%) as a white solid. LCMS m/z=521.2 [M+H]⁺. ¹HNMR (400 MHz, CD₃OD): δ 8.20 (d, J=6.4 Hz, 1H), 7.92 (d, J=3.6 Hz, 1H), 7.38-7.24 (m, 5H), 5.38 (s, 2H), 4.58-3.33 (m, 13H), 1.32-1.28 (m, 2H), 1.23-1.21 (m, 2H).

Step 7: methyl 4-(1-(2-((6-(1-benzyl-1H-pyrazole-4-carbonyl)-2-(1-(trifluoromethyl) cyclopropane-1-carbonyl)-2,6-diazaspiro[3.4]octan-8-yl)methoxy)acetyl) piperidin-4-yl)-3-(cyclohexyloxy)benzoate: To a solution of 2-((6-(1-benzyl-1H-pyrazole-4-carbonyl)-2-(1-(trifluoromethyl)cyclopropane-1-carbonyl)-2,6-diazaspiro [3.4]octan-8-yl) methoxy)acetic acid (145 mg, 0.28 mmol) was added HATU (106 mg, 0.28 mmol) and DIPEA (108 mg, 0.84 mmol). The mixture was stirred for 15 minutes. Methyl 3-(cyclohexyloxy)-4-(piperidin-4-yl)benzoate (106 mg, 0.34 mmol) was added, and stirring was continued for 2 hours. The mixture was diluted with water (50 mL) and extracted with DCM (50 mL×2). The combined organic layers were washed with brine, dried over Na₂SO₄, filtered, and concentrated. The crude product was purified by prep-TLC (eluent: DCM/MeOH=90:7) to afford methyl 4-(1-(2-((6-(1-benzyl-1H-pyrazole-4-carbonyl)-2-(1-(trifluoromethyl)cyclopropane-1-carbonyl)-2,6-diazaspiro[3.4]octan-8-yl) methoxy)acetyl)piperidin-4-yl)-3-(cyclohexyloxy) benzoate (160 mg, 70%) as a yellow oil. LCMS m/z=820.3 [M+H]⁺. ¹H NMR (400 MHz, CD₃OD): δ 8.23-8.16 (m, 1H), 7.94-7.89 (m, 1H), 7.57-7.51 (m, 2H), 7.37-7.18 (m, 6H), 5.49 (s, 2H), 4.63 (d, J=13.2 Hz, 1H), 4.49-4.41 (m, 1H), 4.27 (s, 2H), 4.14-3.50 (m, 12H), 3.28-3.08 (m, 2H), 2.78-2.61 (m, 2H), 2.07-1.37 (m, 16H), 1.27-1.19 (m, 4H).

Step 8: 4-(1-(2-((6-(1-benzyl-1H-pyrazole-4-carbonyl)-2-(1-(trifluoromethyl) cyclopropane-1-carbonyl)-2,6-diazaspiro[3.4]octan-8-yl)methoxy)acetyl)piperidin-4-yl)-3-(cyclohexyloxy)benzoic acid I-60: To a solution of methyl 4-(1-(2-((6-(1-benzyl-1H-pyrazole-4-carbonyl)-2-(1-(trifluoromethyl)cyclopropane-1-carbonyl)-2,6-diazaspiro[3.4]octan-8-yl) methoxy)acetyl)piperidin-4-yl)-3-(cyclohexyloxy)benzoate (160 mg, 0.21 mmol) in a mixture of MeOH and water (2 mL/0.5 mL) was added LiOH (17 mg, 0.41 mmol). The reaction was stirred at room temperature for 2 hours. The mixture was diluted with water (20 mL) and extracted with EtOAc (30 mL). The aqueous phase was collected, acidified with 1 M HCl to pH˜2, and extracted with EtOAc (30 mL×3). The combined organic layers were washed with brine, dried over Na₂SO₄, and concentrated. The residue obtained was purified by prep-HPLC to afford 4-(1-(2-((6-(1-benzyl-1H-pyrazole-4-carbonyl)-2-(1- (trifluoromethyl)cyclopropane-1-carbonyl)-2,6-diazaspiro[3.4]octan-8-yl)methoxy)acetyl)piperidin-4-yl)-3-(cyclohexyloxy)benzoic acid (60 mg, 35%) as a white solid. LCMS m/z=806.2 [M+H]⁺. ¹HNMR (400 MHz, DMSO-d₆): δ 8.32 (d, J=10.8 Hz, 1H), 7.81 (d, 1H), 7.49-7.43 (m, 2H), 7.37-7.18 (m, 6H), 5.37-5.28 (m, 2H), 4.49-4.41 (m, 2H), 4.18 (s, 2H), 3.96 (s, 2H), 3.88-3.78 (m, 3H), 3.73-3.63 (m, 3H), 3.61-3.34 (m, 3H), 3.17-2.95 (m, 2H), 2.69-2.53 (m, 2H), 1.93-1.84 (m, 2H), 1.78-1.64 (m, 4H), 1.61-1.31 (m, 8H), 1.26-1.12 (m, 4H).

### Synthesis of 2-(((6-(5-fluorobenzo[d]thiazol-7-yl)-2-(1-(trifluoromethyl) cyclopropane-1-carbonyl)-2,6-diazaspiro[3.4]octan-8-yl)methoxy)methyl)-6-(4-(trifluoromethyl)cyclohexyl)benzoic acid I-61

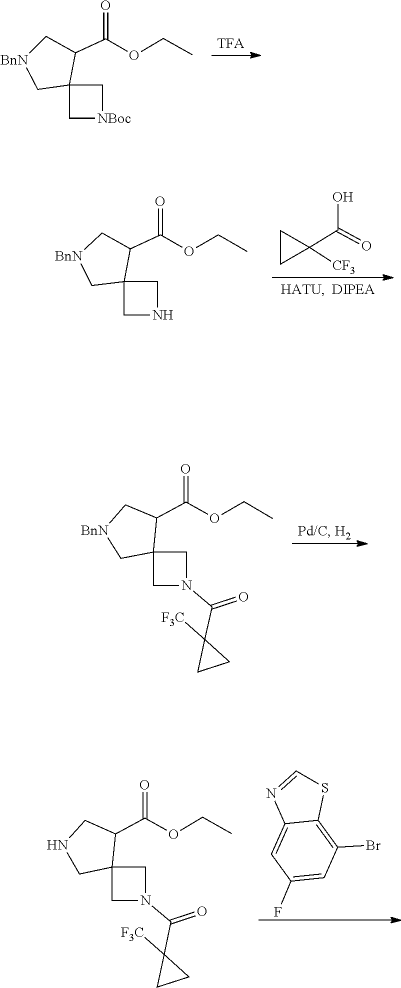

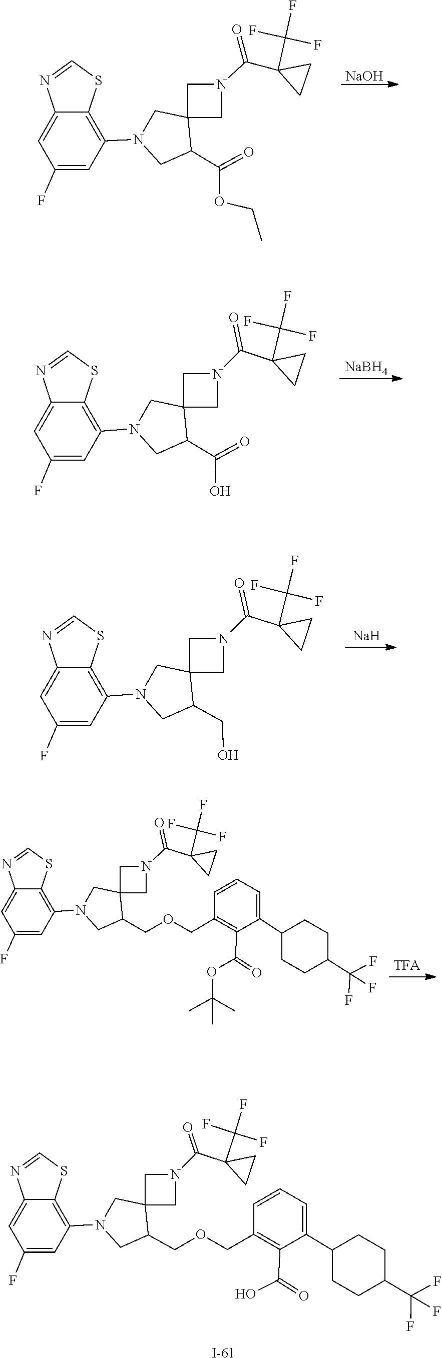

Step 1: ethyl 6-benzyl-2,6-diazaspiro[3.4]octane-8-carboxylate: To a solution of2-(tert-butyl) 8-ethyl 6-benzyl-2,6-diazaspiro[3.4]octane-2,8-dicarboxylate (10.0 g, 26.70 mmol) in DCM (100 mL) was added TFA (30 mL). The reaction was stirred at room temperature for 2 hours. The solvent was removed under vacuum to afford ethyl 6-benzyl-2,6-diazaspiro[3.4]octane-8-carboxylate (7.33 g, quant.) as an oil, which was used directly in the next step. LCMS m/z=274.7 [M+H]⁺. ¹HNMR (400 MHz, CD₃OD): δ 7.31-7.11 (m, 5H), 4.20-4.09 (m, 2H), 4.09-3.96 (m, 4H), 3.95-3.82 (m, 2H), 3.69-3.48 (m, 2H), 3.40-3.36 (m, 1H), 1.08-0.95 (m, 3H).

Step 2: ethyl 6-benzyl-2-(1-(trifluoromethyl)cyclopropane-1-carbonyl)-2,6-diazaspiro[3.4]octane-8-carboxylate: To a solution of solution of 1-(trifluoromethyl) cyclopropane-1-carboxylic acid (4.53 g, 29.4 mmol) in DMF (70 mL) was added HATU (11.17 g, 29.39 mmol) and DIPEA (19.0 mL, 107 mmol). The mixture was stirred at room temperature for 30 minutes. Ethyl 6-benzyl-2,6-diazaspiro[3.4]octane-8-carboxylate (7.33 g, 26.7 mmol) was added, and stirring was continued for another 2 hours. The solvent was removed, and the residue obtained was purified by RP-column (20% H₂O in CH₃CN) to afford ethyl 6-benzyl-2-(1-(trifluoromethyl)cyclopropane-1-carbonyl)-2,6-diazaspiro[3.4]octane-8-carboxylate (10.8 g, 85%) as a yellow oil. LCMS m/z=410.9 [M+H]⁺. ¹H NMR (400 MHz, DMSO-d₆): δ 7.60-7.41 (m, 5H), 4.48-3.39 (m, 13H), 1.20 (dd, J=15.5, 7.4 Hz, 7H).

Step 3: ethyl 2-(1-(trifluoromethyl)cyclopropane-1-carbonyl)-2,6-diazaspiro[3.4]octane-8-carboxylate: To a solution of ethyl 6-benzyl-2-(1-(trifluoromethyl)cyclopropane-1-carbonyl)-2,6-diazaspiro[3.4]octane-8-carboxylate (2.2 g, 5.35 mmol) in methanol (3.0 mL) was added 10% Pd/C (500 mg). The reaction was stirred under a H₂ atmosphere overnight. The catalyst was removed by filtration through Celite, and the filtrate was concentrated to afford ethyl 2-(1-(trifluoromethyl)cyclopropane-1-carbonyl)-2,6-diazaspiro[3.4]octane-8-carboxylate as a yellow oil, which was used without further purification (1.7 g, quant.). LCMS m/z=321.3 [M+H]⁺.

Step 4: ethyl 6-(5-fluorobenzo[d]thiazol-7-yl)-2-(1-(trifluoromethyl)cyclopropane-1-carbonyl)-2,6-diazaspiro[3.4]octane-8-carboxylate: To a solution of ethyl 2-(1-(trifluoromethyl)cyclopropane-1-carbonyl)-2,6-diazaspiro[3.4]octane-8-carboxylate (500 mg, 1.6 mmol) in dioxane (3 mL) was added 3-(benzyloxy)-2-(3-cyclohexylphenyl)propanehydrazide (360 mg, 1.6 mmol), Pd₂(dba)₃ (90 mg, 0.1 mmol), X-phos (150 mg, 0.3 mmol), and Cs₂CO₃ (1 g, 3 mmol). The reaction was heated at 90° C. for 2 hours in a microwave reactor and then diluted with water (20 mL) and extracted with EtOAc (50 mL×2). The combined organic layers were washed with brine, dried over Na₂SO₄, filtered, and concentrated. The residue was purified by RP-column to afford ethyl 6-(5-fluorobenzo[d]thiazol-7-yl)-2-(1-(trifluoromethyl)cyclopropane-1-carbonyl)-2,6-diazaspiro[3.4]octane-8-carboxylate (400 mg, 55%) as a brown solid. LCMS m/z=472.1 [M+H]⁺. ¹HNMR (400 MHz, Methanol-d₄): δ 9.16 (s, 1H), 7.10 (dd, J=9.0, 2.2 Hz, 1H), 6.40 (dd, J=12.0, 2.2 Hz, 1H), 4.61-3.42 (m, 11H), 1.31-1.21 (m, 7H).

Step 5: 6-(5-fluorobenzo[d]thiazol-7-yl)-2-(1-(trifluoromethyl)cyclopropane-1-carbonyl)-2,6-diazaspiro[3.4]octane-8-carboxylic acid: To a solution of ethyl 6-(5-fluorobenzo[d]thiazol-7-yl)-2-(1-(trifluoromethyl)cyclopropane-1-carbonyl)-2,6-diazaspiro[3.4]octane-8-carboxylate (530 mg, 1.1 mmol) in MeOH (5 mL) was added 10% NaOH (0.5 mL). The reaction was stirred at room temperature for 2 hours and then diluted with water (20 mL) and extracted with ether (20 mL). The aqueous layer was collected and acidified to pH 2 with 1 M HCl. The aqueous solution was extracted with EtOAc (50 mL×2), and the combined organic layers were washed with brine, dried over Na₂SO₄, filtered, and concentrated to afford 6-(5-fluorobenzo[d]thiazol-7-yl)-2-(1-(trifluoromethyl)cyclopropane-1-carbonyl)-2,6-diazaspiro[3.4]octane-8-carboxylic acid (430 mg, 86%) as a brown solid. LCMS m/z=444.0 [M+H]⁺.

Step 6: (6-(5-fluorobenzo[d]thiazol-7-yl)-8-(hydroxymethyl)-2,6-diazaspiro[3.4]octan-2-yl)(1-(trifluoromethyl)cyclopropyl)methanone: To a solution of 6-(5-fluorobenzo[d]thiazol-7-yl)-2-(1-(trifluoromethyl)cyclopropane-1-carbonyl)-2,6-diazaspiro[3.4]octane-8-carboxylic acid (100 mg, 0.2 mmol) in THF (1 mL) at 0° C. was added isobutyl chloroformate (43 mg, 0.3 mmol) and N-methyl morpholine (30 mg, 0.3 mmol). The reaction was stirred for 30 minutes. A solution of NaBH₄ (25 mg, 0.6 mmol) in water (1 mL) was added dropwise, and stirring was continued at 0° C. for 1 hour. The reaction was diluted with water (30 mL) and extracted with EtOAc (50 mL×3). The combined organic layers were washed with brine, dried over Na₂SO₄, filtered, and concentrated. The residue was purified by RP-column to afford (6-(5-fluorobenzo[d]thiazol-7-yl)-8-(hydroxymethyl)-2,6-diazaspiro[3.4]octan-2-yl)(1-(trifluoromethyl)cyclopropyl)methanone (50 mg, 52%) as a brown solid. LCMS m/z=430.0 [M+H]⁺.

Step 7: tert-butyl 2-(((6-(5-fluorobenzo[d]thiazol-7-yl)-2-(1-(trifluoromethyl) cyclopropane-1-carbonyl)-2,6-diazaspiro[3.4]octan-8-yl)methoxy)methyl)-6-(4-(trifluoromethyl)cyclohexyl)benzoate: To a solution of (6-(5-fluorobenzo[d]thiazol-7-yl)-8-(hydroxymethyl)-2,6-diazaspiro[3.4]octan-2-yl)(1-(trifluoromethyl)cyclopropyl)methanone (100 mg, 0.2 mmol) and tert-butyl 2-(bromomethyl)-6-(4-(trifluoromethyl)cyclohexyl)benzoate (117 mg, 0.28 mmol) in THF (2 mL) at 0° C. was added NaH (8.4 mg, 0.35 mmol). The reaction was stirred at room temperature overnight. The mixture was diluted with water (20 mL) and extracted with EtOAc (50 mL×2). The combined organic layers were washed with brine, dried over Na₂SO₄, filtered, and concentrated. The residue was purified by prep-TLC to afford tert-butyl 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (25 mg, 14%) as a white solid. LCMS m/z=770.1 [M+H]⁺. ¹H NMR (400 MHz, Chloroform-d): δ 8.93 (s, 1H), 7.33-7.27 (m, 1H), 7.25-7.16 (m, 3H), 6.27 (dd, J=11.8, 2.2 Hz, 1H), 4.54 (s, 2H), 4.36-3.46 (m, 11H), 2.74-2.59 (m, 2H), 2.43-2.31 (m, 1H), 2.17-2.00 (m, 3H), 1.80-1.66 (m, 5H), 1.56 (s, 9H), 1.19-1.11 (m, 4H). an alternative batch was prepared they gave the following data: ¹H NMR (400 MHz, Methanol-d₄) δ 9.17 (s, 1H), 7.35-7.19 (m, 3H), 7.09 (dd, J=9.0, 2.2 Hz, 1H), 6.38 (dd, J=12.1, 2.2 Hz, 1H), 4.60-4.54 (m, 2H), 4.43-3.99 (m, 3H), 3.85-3.78 (m, 3H), 3.70-3.58 (m, 2H), 3.56-3.50 (m, 1H), 2.77-2.62 (m, 2H), 2.51-2.36 (m, 1H), 2.16-1.89 (m, 3H), 1.75-1.70 (m, 4H), 1.58 (s, 9H), 1.45-1.25 (m, 2H), 1.22-1.11 (m, 4H). m/z=770.1 [M+H]⁺

Step 8: 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl) cyclohexyl) benzoic acid I-61: To a solution of tert-butyl 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (22 mg, 0.03 mmol) in DCM (1 mL) was added TFA (1 mL). The reaction was stirred at room temperature for 2 hours. The solvent was removed under vacuum, and the residue obtained was purified by prep-TLC (eluent: DCM/MeOH=30:1) to afford 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (15 mg, 75%) as an off-white solid. LCMS m/z=713.9 [M+H]⁺. ¹H NMR (400 MHz, Methanol-d₄): δ 9.15 (d, J=2.2 Hz, 1H), 7.34-7.17 (m, 3H), 7.12-7.05 (m, 1H), 6.38 (dd, J=12.1, 2.4 Hz, 1H), 4.61 (d, J=13.1 Hz, 2H), 4.34-3.45 (m, 10H), 2.87-2.66 (m, 2H), 2.48-2.35 (m, 1H), 2.21-1.91 (m, 3H), 1.80-1.69 (m, 4H), 1.66-1.34 (m, 2H), 1.25-1.01 (m, 4H).

### Synthesis of 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid I-52

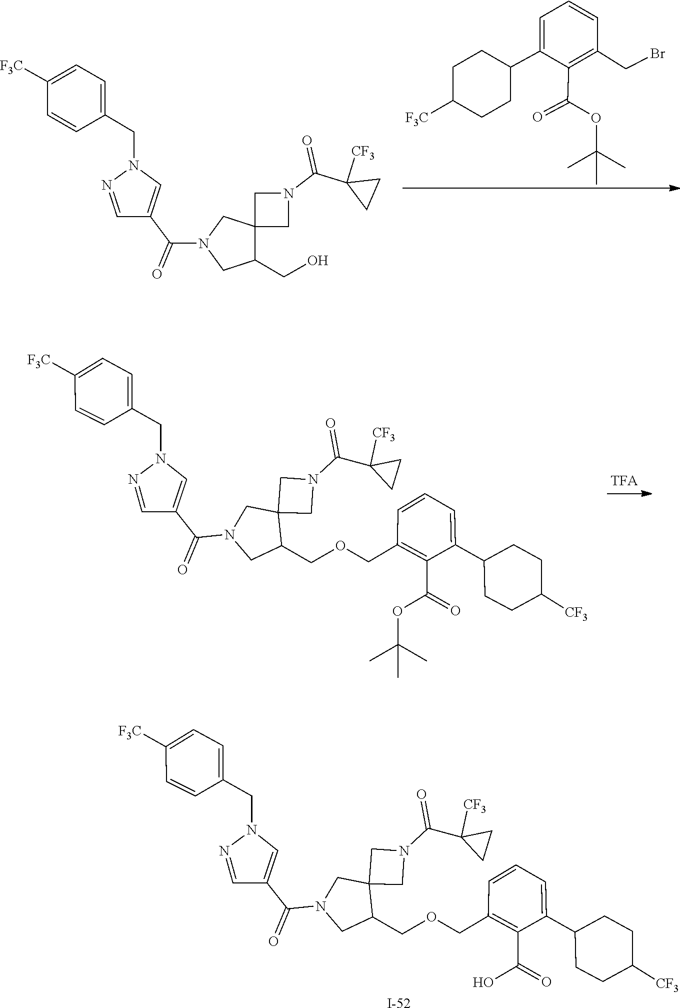

Step 1: tert-butyl 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate: To a solution of tert-butyl 2-(bromomethyl)-6-(4-(trifluoromethyl)cyclohexyl)benzoate (80 mg, 0.2 mmol) and (8-(hydroxymethyl)-2-(1-(trifluoromethyl)cyclopropane-1-carbonyl)-2,6-diazaspiro[3.4]octan-6-yl)(1-(4-(trifluoromethyl) benzyl)-1H-pyrazol-4-yl)methanone (111 mg, 0.2 mmol) in THF (2 mL) was added NaH (6 mg, 0.22 mmol). The reaction was stirred at room temperature overnight. The reaction was poured into ice-water (20 mL) and extracted with EtOAc (50 mL×2). The combined organic layers were washed with brine, dried over Na₂SO₄, filtered, and concentrated. The residue was purified by prep-TLC to afford tert-butyl 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl) methoxy)methyl)-6-(4-(trifluoromethyl)cyclohexyl)benzoate (80 mg, 48%) as a white solid. LCMS m/z=871.2 [M+H]⁺. ¹H NMR (400 MHz, CD₃OD): δ 8.27 (d, J=10.0 Hz, 1H), 7.93 (d, J=6.7 Hz, 1H), 7.65 (d, J=7.5 Hz, 2H), 7.43 (d, J=7.9 Hz, 2H), 7.33-7.29 (m, 1H), 7.27-7.20 (m, 2H), 5.48 (s, 2H), 4.54 (s, 2H), 4.15-3.47 (m, 9H), 2.78-2.63 (m, 2H), 2.42 (d, J=18.1 Hz, 2H), 1.74 (d, J=6.5 Hz, 7H), 1.61 (s, 9H), 1.17 (s, 4H).

Step 2: 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid I-52: To a solution of tert-butyl 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl) benzoate (180 mg, 0.2 mmol) in DCM (2 mL) was added TFA (lmL). The reaction was stirred at room temperature for 2 hours. The solvent was removed, and the residue obtained was purified by prep-HPLC to afford 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (120 mg, 71%) as a white solid. LCMS m/z=815.1 [M+H]⁺. ¹H NMR (400 MHz, CD3OD): δ 8.34 (d, J=6.4 Hz, 1H), 7.94 (d, J=8.5 Hz, 1H), 7.65 (dd, J=8.4, 2.8 Hz, 2H), 7.43 (d, J=7.9 Hz, 2H), 7.35-7.16 (m, 3H), 5.48 (s, 2H), 4.59 (s, 3H), 4.43-4.19 (m, 2H), 4.11-3.89 (m, 3H), 3.80-3.58 (m, 4H), 2.87-2.58 (m, 2H), 2.43 (s, 1H), 2.04 (dt, J=27.9, 13.7 Hz, 3H), 1.72 (q, J=15.8, 11.4 Hz, 4H), 1.16 (d, J=13.3 Hz, 4H). An 2^{nd} batch was synthesized which gave the following data: ¹H NMR (400 MHz, Methanol-d₄) δ 8.37-8.24 (m, 1H), 7.94 (d, J=9.9 Hz, 1H), 7.70-7.62 (m, 2H), 7.44 (d, J=7.9 Hz, 2H), 7.37-7.16 (m, 3H), 5.48 (s, 2H), 4.66-3.45 (m, 13H), 2.86-2.58 (m, 2H), 2.46-1.93 (m, 4H), 1.79-1.70 (m, 3H), 1.62-1.33 (m, 1H), 1.25-1.05 (m, 4H). m/z=815.1 [M+H]⁺

### Synthesis of 6-(1-(4-cyanobenzyl)-1H-pyrazole-4-carbonyl)-2-(1-(trifluoromethyl) cyclopropane-1-carbonyl)-N-((3-(1-(trifluoromethyl)cyclopropyl)phenyl)sulfonyl)-2,6-diazaspiro[3.4]octane-8-carboxamide I-59

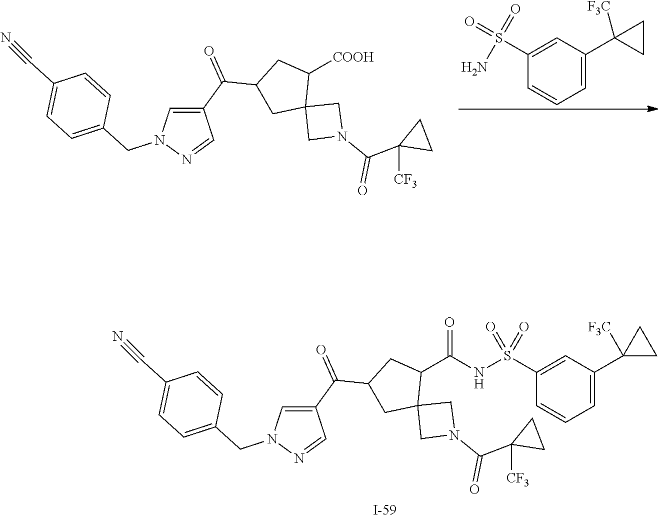

To a solution of 6-(1-(4-cyanobenzyl)-1H-pyrazole-4-carbonyl)-2-(1-(trifluoromethyl) cyclopropane-1-carbonyl)-2,6-diazaspiro[3.4]octane-8-carboxylic acid (110 mg, 0.22 mmol) in DCM (2 mL) was added HATU (83 mg, 0.22 mmol) and DIPEA (85 mg, 0.66 mmol). The mixture was stirred at room temperature for 10 minutes. 3-(1-(Trifluoromethyl)cyclopropyl) benzenesulfonamide (64 mg, 0.24 mmol) was added, and stirring was continued for 2 hours. The mixture was diluted with water (30 mL) and extracted with DCM (50 mL×3). The combined organic layers were washed with brine, dried over Na₂SO₄, filtered, and concentrated. The residue was purified by column chromatography on silica gel (eluent: DCM/MeOH=30:1) and RP-column (30% water in ACN) to afford 6-(1-(4-cyanobenzyl)-1H-pyrazole-4-carbonyl)-2-(1-(trifluoromethyl)cyclopropane-1-carbonyl)-N-((3-(1-(trifluoromethyl)cyclopropyl)phenyl) sulfonyl)-2,6-diazaspiro[3.4]octane-8-carboxamide (59 mg, 35%) as a yellow solid. LCMS m/z=749.0 [M+H]⁺. ¹H NMR (400 MHz, Methanol-d4): δ 8.23 (d, J=18.2 Hz, 1H), 8.13 (s, 1H), 7.99 (t, J=7.2 Hz, 1H), 7.88 (d, J=16.4 Hz, 1H), 7.80 (d, J=7.8 Hz, 1H), 7.72 (d, J=8.0 Hz, 2H), 7.63-7.55 (m, 1H), 7.47-7.36 (m, 2H), 5.47 (s, 2H), 4.40-3.67 (m, 8H), 3.20 (s, 1H), 1.43 (d, J=8.2 Hz, 2H), 1.20 (s, 4H), 1.13 (s, 2H).

### Synthesis of (S)—N-((3-(tetrahydro-2H-pyran-4-yl)phenyl)sulfonyl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide I-58

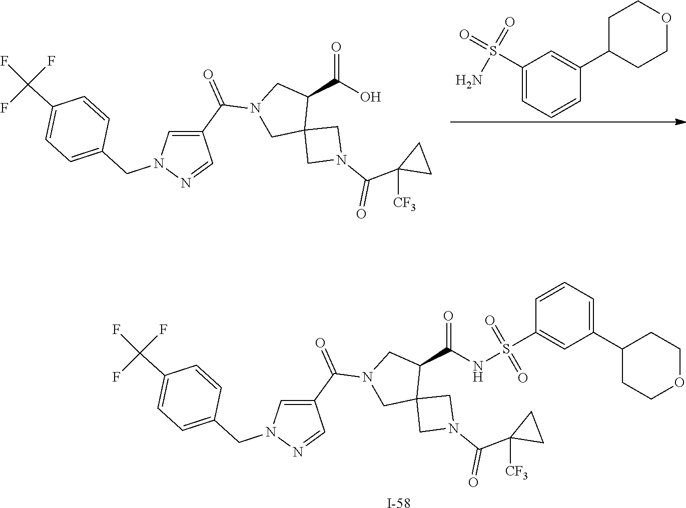

To a solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (112 mg, 0.2 mmol) in DMF (1.0 mL) was added HATU (94 mg, 0.25 mmol) and DIPEA (80 mg, 0.6 mmol). The mixture was stirred at room temperature for 15 minutes. 3-(Tetrahydro-2H-pyran-4-yl)benzenesulfonamide (50 mg, 0.2 mmol) was added, and stirring was continued at room temperature overnight. The mixture was diluted with water (30 mL) and extracted with EtOAc (50 mL×3). The combined organic layers were washed with brine, dried over Na₂SO₄, filtered, and concentrated. The residue was purified by prep-HPLC to afford (S)—N-((3-(tetrahydro-2H-pyran-4-yl)phenyl)sulfonyl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8- carboxamide (32 mg, 45%) as a white solid. LCMS m/z=768.0 [M+H]⁺. ¹H NMR (400 MHz, CD₃OD): δ 8.22 (d, J=19.1 Hz, 1H), 7.91-7.79 (m, 3H), 7.65 (d, J=8.1 Hz, 2H), 7.59-7.55 (m, 1H), 7.54-7.46 (m, 1H), 7.42 (d, J=8.0 Hz, 2H), 5.46 (s, 2H), 4.29 (s, 2H), 4.01 (q, J=10.2, 9.1 Hz, 5H), 3.88-3.70 (m, 3H), 3.53 (p, J=7.1, 6.4 Hz, 2H), 3.25-3.17 (m, 1H), 2.89 (t, J=8.0 Hz, 1H), 1.79-1.71 (m, 4H), 1.19 (s, 4H).

### Synthesis of Building Block: 6-(4-(trifluoromethyl)phenyl)pyridine-2-thiol

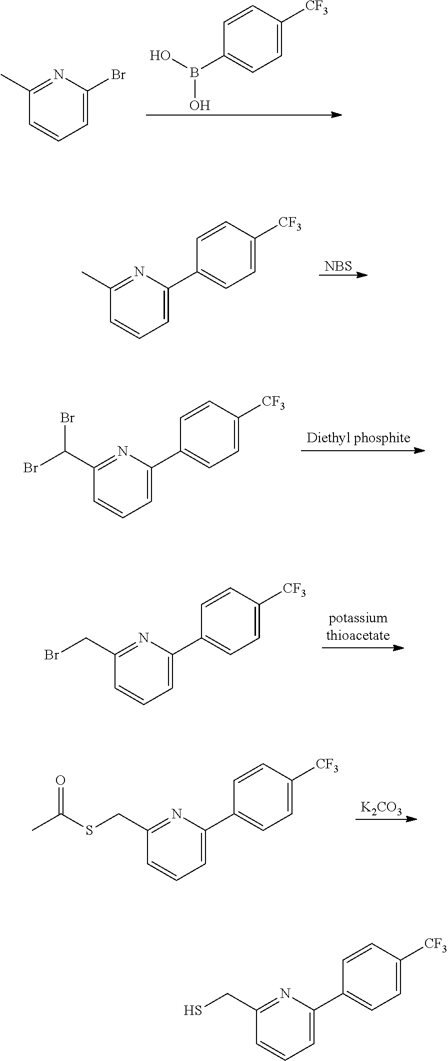

Step 1: 2-methyl-6-(4-(trifluoromethyl)phenyl)pyridine: To a solution of 2-bromo-6-methylpyridine (1.0 g, 5.81 mmol) in a mixture of 1,4-dioxane and water (10 mL and 2 mL) under a N₂ atmosphere was added Pd(PPh₃)₄ (335 mg, 0.29 mmol), (4-(trifluoromethyl)phenyl)boronic acid (1.32 g, 6.98 mmol), and sodium carbonate (1.44 g, 11.63 mmol). The reaction was heated at reflux for 4 hours and then diluted with water (20 mL) and extracted with EtOAc (30 mL×2). The combined organic layers were washed with brine, dried over Na₂SO₄, filtered, and concentrated. The residue was purified by column chromatography on silica gel (petroleum ether/EtOAc=50:1) to afford 2-methyl-6-(4-(trifluoromethyl)phenyl)pyridine (870 mg, 63%) as a white solid. ¹H NMR (400 MHz, CD₃OD): δ 8.14 (d, J=8.2 Hz, 2H), 7.85-7.66 (m, 4H), 7.28 (d, J=7.7 Hz, 1H), 2.61 (s, 3H).

Step 2: 2-(dibromomethyl)-6-(4-(trifluoromethyl)phenyl)pyridine: To a solution of 2-methyl-6-(4-(trifluoromethyl)phenyl)pyridine (6.8 g, 28.7 mmol) in CCl₄ (68 mL) was added AIBN (2.0 g, 12.32 mmol) and NBS (18.4 g, 103.12 mmol). The reaction was heated at reflux for 6 hours. The mixture was diluted with water (100 mL) and extracted with DCM (100 mL×2). The combined organic layers were washed with brine, dried over Na₂SO₄, filtered, and concentrated. The crude product was purified by column chromatography on silica gel (petroleum ether/DCM=5:1) to afford 2-(dibromomethyl)-6-(4-(trifluoromethyl)phenyl)pyridine (6.49 g, 57%) as a white solid. 1H NMR (400 MHz, CD₃OD): δ 8.29 (d, J=8.2 Hz, 2H), 8.03-7.89 (m, 2H), 7.83-7.75 (m, 311), 7.03 (s, 1H).

Step 3: 2-(bromomethyl)-6-(4-(trifluoromethyl)phenyl)pyridine: To a solution of 2-(dibromomethyl)-6-(4-(trifluoromethyl)phenyl)pyridine (6.49 g, 17.3 mmol) in acetonitrile (50 mL) at 0° C. was added DIPEA (4.5 mL, 26 mmol) and phosphonic acid diethyl ester (3.59 g, 26 mmol). The mixture was stirred for 30 minutes and then warmed to room temperature and stirred for an additional 3 hours. The reaction was diluted with water (50 mL) and extracted with EtOAc (80 mL×2). The combined organic layers were washed with brine, dried over Na₂SO₄, filtered, and concentrated. The residue was purified by column chromatography on silica gel (petroleum ether/EtOAc=30:1) to afford 2-(bromomethyl)-6-(4-(trifluoromethyl)phenyl)pyridine (3.1 g, 60%) as a white solid. ¹H NMR (400 MHz, CD₃OD): δ 8.24 (d, J=8.2 Hz, 2H), 7.95-7.83 (m, 2H), 7.79 (d, J=8.2 Hz, 2H), 7.55 (d, J=8.6 Hz, 1H), 4.68 (s, 2H).

Step 4: S-(6-(4-(trifluoromethyl)phenyl)pyridin-2-yl) ethanethioate: To a solution of 2-(bromomethyl)-6-(4-(trifluoromethyl)phenyl)pyridine (200 mg, 0.64 mmol) in methanol (4 mL) was added TEA (128 mg, 1.28 mmol) and potassium thioacetate (72 mg, 0.64 mmol). The reaction was stirred at room temperature for 30 minutes and then diluted with water (10 mL) and extracted with EtOAc (20 mL×3). The combine organic phases were washed with brine, dried over Na₂SO₄, filtered, and concentrated purified by prep-TLC (petroleum ether/EtOAc=3:1) to afford S-(6-(4-(trifluoromethyl)phenyl)pyridin-2-yl) ethanethioate (100 mg, 51%) as a colorless oil. LCMS m/z=312.1 [M+H]⁺. ¹H NMR (400 MHz, CD₃OD): δ 8.28 (d, J=8.2 Hz, 2H), 8.00-7.84 (m, 4H), 7.42 (d, J=7.5 Hz, 1H), 4.32 (s, 2H), 2.39 (s, 3H).

Step 5: 6-(4-(trifluoromethyl)phenyl)pyridine-2-thiol: To a solution of S-(6-(4-(trifluoromethyl)phenyl)pyridin-2-yl) ethanethioate (100 mg, 0.32 mmol) in a mixture of methanol and water (3 mL/1 mL) was added potassium carbonate (67 mg, 0.48 mmol). The reaction was heated at 85° C. for 1.5 hours and then filtered through Celite. The filtrate was diluted with water, the pH adjusted to 2 with 1 N HCl, and the aqueous layer was extracted with EtOAc (30 mL×2). The combined organic layers were washed with brine, dried over Na₂SO₄, filtered, and concentrated to afford 6-(4-(trifluoromethyl)phenyl)pyridine-2-thiol (77 mg, 89%) as a white solid. LCMS m/z=270.1 [M+H]⁺. ¹H NMR (400 MHz, DMSO-d₆): δ 8.31 (d, J=8.2 Hz, 2H), 7.97-7.83 (m, 4H), 7.49 (dd, J=6.6, 2.2 Hz, 1H), 3.91 (d, J=6.2 Hz, 2H), 3.08-2.94 (m, 1H).

### Synthesis of Building Block: 2-(3-chloro-4-(trifluoromethyl)benzyl)oxirane

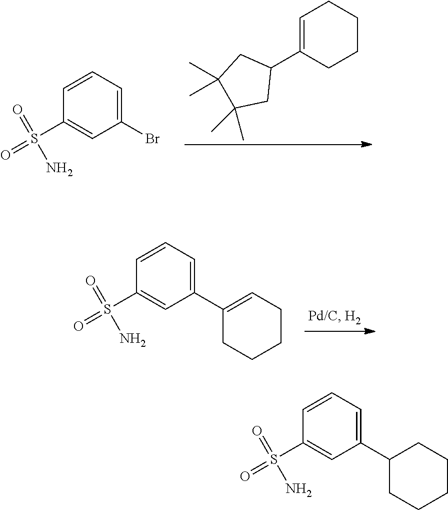

Step 1: 2′,3′,4′,5′-tetrahydro-[1,1′-biphenyl]-3-sulfonamide: To a solution of 3-bromobenzenesulfonamide (100 mg, 0.42 mmol) in a mixture of dioxane (1.5 mL) and water (0.5 mL) was added 2-(cyclohex-1-en-1-yl)-4,4,5,5-tetramethyl-1,3,2-dioxaborolane (97 mg, 0.47 mmol), K₂CO₃ (117 mg, 0.85 mmol), and Pd(PPh₃)₄ (24 mg, 21 μmol). The reaction was heated at 100° C. under a N₂ atmosphere for 4 hours and then diluted with water (30 mL) and extracted with EtOAc (75 mL×3). The combined organic layers were washed with brine, dried over Na₂SO₄, filtered, and concentrated. The residue was purified by silica gel column (eluent: petroleum ether: EtOAc=30:1-10:1) to afford 2′,3′,4′,5′-tetrahydro-[1,1′-biphenyl]-3-sulfonamide (80 mg, 80%) as a white solid. ¹H NMR (400 MHz, CDCl₃): δ 7.79 (s, 1H), 7.58 (dd, J=20.4, 7.8 Hz, 2H), 7.44 (t, J=7.8 Hz, 1H), 6.20 (s, 1H), 2.37-2.27 (m, 2H), 2.19-2.10 (m, 2H), 1.74-1.64 (m, 2H), 1.60-1.51 (m, 2H).

Step 2: 2-(3-chloro-4-(trifluoromethyl)benzyl)oxirane: To a solution of 2′,3′,4′,5′-tetrahydro-[1,1′-biphenyl]-3-sulfonamide (80 mg, 0.33 mmol) in MeOH (1 mL) was added 10% Pd/C (10 mg). The reaction was stirred at room temperature under H₂ atmosphere for 2 hours. The catalyst was removed by filtration through Celite, and the filtrate was concentrated to afford 3-cyclohexylbenzenesulfonamide (70 mg, 88%) as a white solid. ¹H NMR (400 MHz, CD₃OD): δ 7.77 (s, 1H), 7.74-7.67 (m, 1H), 7.47-7.40 (m, 2H), 2.66-2.55 (m, 1H), 1.92-1.80 (m, 4H), 1.81-1.72 (m, 1H), 1.55-1.24 (m, 4H), 1.37-1.24 (m, 1H)

### Synthesis of Building Block: methyl 3-(cyclohexyloxy)-4-(piperidin-4-yl)benzoate

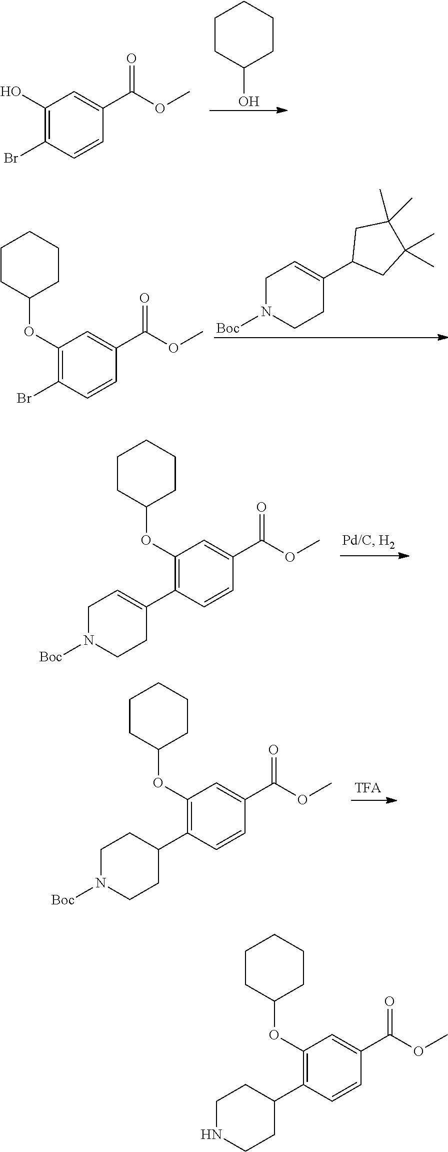

Step 1: methyl 4-bromo-3-(cyclohexyloxy)benzoate: To a solution of methyl 4-bromo-3-hydroxybenzoate (1.0 g, 4.33 mmol) in anhydrous THF (45 mL) at 0° C. under a N₂ atmosphere was added cyclohexanol (1.3 g, 12.98 mmol) and PPh₃ (3.4 g, 12.98 mmol). The reaction mixture was stirred at 0° C. for 30 minutes. Next, DIAD (2.6 g, 12.98 mmol) was added, and the reaction was allowed to warm to room temperature and stirred for 2 hours. The reaction was quenched with water (200 mL) and extracted with EtOAc (100 mL). The combined organic layers were washed with brine, dried over Na₂SO₄, filtered, and concentrated. The residue was purified by column chromatography on silica gel (eluent: petroleum ether/EtOAc=50:1) to afford methyl 4-bromo-3-(cyclohexyloxy)benzoate (1.3 g, quant) as a yellow oil. ¹HNMR (400 MHz, CD₃OD): δ 7.64-7.61 (m, 1H), 7.57-7.55 (m, 1H), 7.47-7.43 (m, 1H), 4.52-4.44 (m, 1H), 3.89 (s, 3H), 1.96-1.88 (m, 2H), 1.87-1.78 (m, 2H), 1.70-1.60 (m, 2H), 1.53-1.38 (m, 4H).

Step 2: tert-butyl 4-(2-(cyclohexyloxy)-4-(methoxycarbonyl)phenyl)-3,6-dihydropyridine-1(2H)-carboxylate: To a solution of methyl 4-bromo-3-(cyclohexyloxy) benzoate (1.2 g, 3.83 mmol) in a mixture of dioxane and water (10.0 mL/2.0 mL) was added Pd(dppf)Cl₂ (276 mg, 0.38 mmol), tert-butyl 4-(4,4,5,5-tetramethyl-1,3,2-dioxaborolan-2-yl)-3,6-dihydropyridine-1(2H)-carboxylate (1.4 g, 4.60 mmol), and Na₂CO₃ (950 mg, 7.66 mmol). The reaction was heated at 100° C. overnight. The mixture was diluted with water (100 mL) and extracted with EtOAc (100 mL×3). The combined organic layers were washed with brine, dried over Na₂SO₄, filtered, and concentrated. The residue obtained was purified by column chromatography on silica gel (eluent: petroleum ether/EtOAc=50:1) to afford tert-butyl 4-(2-(cyclohexyloxy)-4-(methoxycarbonyl)phenyl)-3,6-dihydropyridine-1(2H)-carboxylate (1.4 g, 87%) as a yellow oil. LCMS m/z=316.1 [M-100+H]⁺. ¹H NMR (400 MHz, DMSO-d₆): δ 7.51-7.45 (m, 2H), 7.26 (d, J=7.8 Hz, 1H), 5.84 (s, 1H), 4.48-4.39 (m, 1H), 3.97 (s, 21), 3.84 (s, 3H), 3.53-3.43 (m, 2H), 2.43 (s, 2H), 1.91-1.82 (m, 2H), 1.71-1.62 (m, 2H), 1.54-1.45 (m, 3H), 1.43 (s, 9H), 1.41-1.26 (m, 3H).

Step 3: tert-butyl 4-(2-(cyclohexyloxy)-4-(methoxycarbonyl)phenyl)piperidine-1-carboxylate: To a solution of tert-butyl 4-(2-(cyclohexyloxy)-4-(methoxycarbonyl)phenyl)-3,6-dihydropyridine-1(2H)-carboxylate (200 mg, 0.48 mmol) in EtOH (2 mL) was added 10% Pd/C (80 mg). The reaction was stirred under a H₂ atmosphere for 4 hours. The catalyst was removed by filtration through Celite, and the filtrate was concentrated to afford tert-butyl 4-(2-(cyclohexyloxy)-4-(methoxycarbonyl)phenyl)piperidine-1-carboxylate (180 mg, 89%) as a yellow oil, which was used directly in the next step. LCMS m/z=440.2 [M+Na]⁺. ¹H NMR (400 MHz, CD₃OD): δ 7.56-7.53 (m, 1H), 7.52-7.50 (m, 1H), 7.26-7.24 (m, 1H), 4.47-4.41 (m, 1H), 4.25-4.19 (m, 2H), 3.88 (s, 3H), 3.21-3.12 (m, 1H), 2.91-2.80 (m, 2H), 2.01-1.93 (m, 2H), 1.84-1.74 (m, 5H), 1.69-1.55 (m, 6H), 1.53-1.49 (m, 1H), 1.48 (s, 9H).

Step 4: methyl 3-(cyclohexyloxy)-4-(piperidin-4-yl)benzoate: To a solution of tert-butyl 4-(2-(cyclohexyloxy)-4-(methoxycarbonyl)phenyl)piperidine-1-carboxylate (180 mg, 0.43 mmol) in DCM (2 mL) was added TFA (1 mL). The mixture was stirred at room temperature for 2 hours. The solvent was removed under vacuum to afford methyl 3-(cyclohexyloxy)-4-(piperidin-4-yl)benzoate, which was used directly in the next step. LCMS m/z=318.2 [M+H]⁺.

### Synthesis of Building Block: tert-butyl 2-(bromomethyl)-6-(4-(trifluoromethyl) cyclohexyl)benzoate

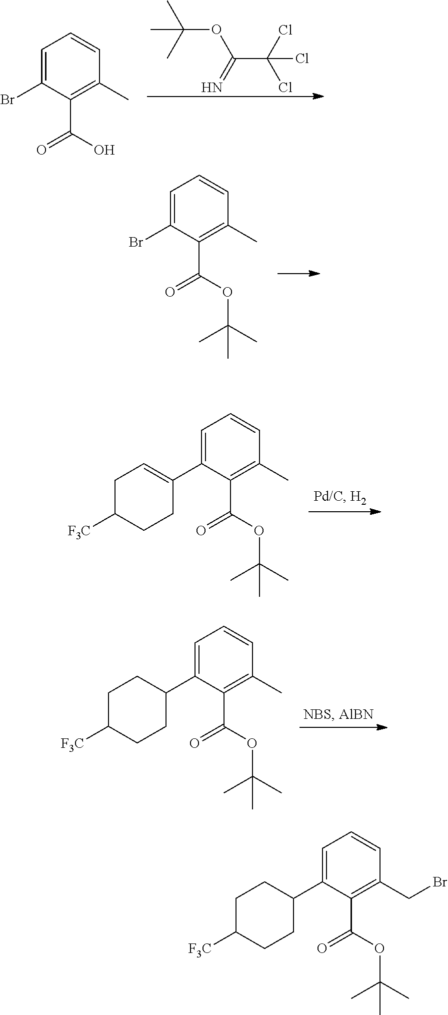

Step 1: tert-butyl 2-bromo-6-methylbenzoate: To a solution of 2-bromo-6-methylbenzoic acid (5 g, 23.2 mmol) in THF (50 mL) at 0° C. was added tert-butyl 2,2,2-trichloroacetimidate (10.2 g, 46.5 mmol) and BF₃·Et₂O (46.5% in Et₂O, 6.2 g, 46.5 mmol). The reaction was stirred at room temperature overnight and then diluted with water (30 mL) and extracted with EtOAc (50 mL×3). The combined organic layers were washed with brine, dried over Na₂SO₄, filtered, and concentrated. The residue was purified by column chromatography on silica gel (eluent: EtOAc/petroelum ether=1:15) to afford tert-butyl 2-bromo-6-methylbenzoate (3.7 g, 59%) as a colorless oil. ¹H NMR (400 MHz, CD₃OD): δ 7.40 (dd, J=7.2, 1.8 Hz, 1H), 7.23-7.16 (m, 2H), 2.33 (s, 3H), 1.61 (s, 9H).

Step 2: tert-butyl 3-methyl-4′-(trifluoromethyl)-2′,3′,4′,5′-tetrahydro-[1,1′-biphenyl]-2-carboxylate: To a solution of 1-(3-bromo-5-fluorophenyl)thiourea (200 mg, 0.74 mmol) in a mixture of dioxane and water (3 mL/1 mL) was added 4,4,5,5-tetramethyl-2-(4-(trifluoromethyl) cyclohex-1-en-1-yl)-1,3,2-dioxaborolane (204 mg, 0.74 mmol), Pd(PPh₃)₄ (85 mg, 0.07 mmol), and K₂CO₃ (204 mg, 1.48 mmol). The reaction was heated at 100° C. under a N₂ atmosphere overnight. The mixture was diluted with water (30 mL) and extracted with EtOAc (50 mL×3). The combined organic layers were washed with brine, dried over Na₂SO₄, filtered, and concentrated. The residue obtained was purified by column chromatography on silica gel (eluent: petroleum ether/EtOAc=30:1) to afford tert-butyl 3-methyl-4′-(trifluoromethyl)-2′,3′,4′,5′-tetrahydro-[1,1′-biphenyl]-2-carboxylate (230 mg, 92%) as a colorless oil. ¹H NMR (400 MHz, CD₃OD): δ 7.24 (t, J=7.7 Hz, 1H), 7.11 (d, J=7.6 Hz, 1H), 7.02 (d, J=7.7 Hz, 1H), 5.59 (d, J=5.0 Hz, 1H), 2.43 (m, 7.9 Hz, 3H), 2.32 (s, 3H), 2.24-2.08 (m, 2H), 1.72-1.59 (m, 2H), 1.55 (s, 9H).

Step 3: tert-butyl 2-methyl-6-(4-(trifluoromethyl)cyclohexyl)benzoate: To a solution of tert-butyl 3-methyl-4′-(trifluoromethyl)-2′,3′,4′,5′-tetrahydro-[1,1′-biphenyl]-2-carboxylate (230 mg, 0.6 mmol) in MeOH (2 mL) was added 10% Pd/C (20 mg). The reaction was stirred at room temperature under H₂ atmosphere for 2 hours. The catalyst was removed by filtration through Celite, and the filtrate was concentrated to afford tert-butyl 2-methyl-6-(4-(trifluoromethyl)cyclohexyl)benzoate (170 mg, 74%) as a colorless oil. ¹H NMR (400 MHz, CD₃OD): δ 7.24 (t, J=7.7 Hz, 1H), 7.10 (d, J=7.9 Hz, 1H), 7.05 (d, J=7.6 Hz, 1H), 2.66 (d, J=6.3 Hz, 1H), 2.54-2.39 (m, 1H), 2.29 (s, 3H), 2.23-1.92 (m, 3H), 1.74 (d, J=6.4 Hz, 5H), 1.62 (s, 9H).

Step 4: tert-butyl 2-(bromomethyl)-6-(4-(trifluoromethyl)cyclohexyl)benzoate: To a solution of tert-butyl 2-methyl-6-(4-(trifluoromethyl)cyclohexyl)benzoate (230 mg, 0.67 mmol) in CCl₄ (4.0 mL) was added NBS (143 mg, 0.8 mmol) and AIBN (22 mg, 0.13 mmol). The reaction was stirred at 85° C. for 1 hour. The mixture was diluted with water (50 mL) and extracted with DCM (30 mL×3). The combined organic layers were washed with brine, dried over Na₂SO₄, filtered, and concentrated. The residue obtained was purified by prep-TLC (eluent: petroleum ether/EtOAc=40:1) to afford tert-butyl 2-(bromomethyl)-6-(4-(trifluoromethyl)cyclohexyl) benzoate (131 mg, 47%) as a colorless oil. ¹H NMR (400 MHz, CD₃OD): δ 7.39-7.34 (m, 1H), 7.31-7.25 (m, 2H), 4.58 (s, 2H), 2.16-1.91 (m, 4H), 1.79-1.71 (m, 6H), 1.66 (s, 9H).

### Synthesis of Building Block: 3-(1-(trifluoromethyl)cyclopropyl)benzenesulfonamide

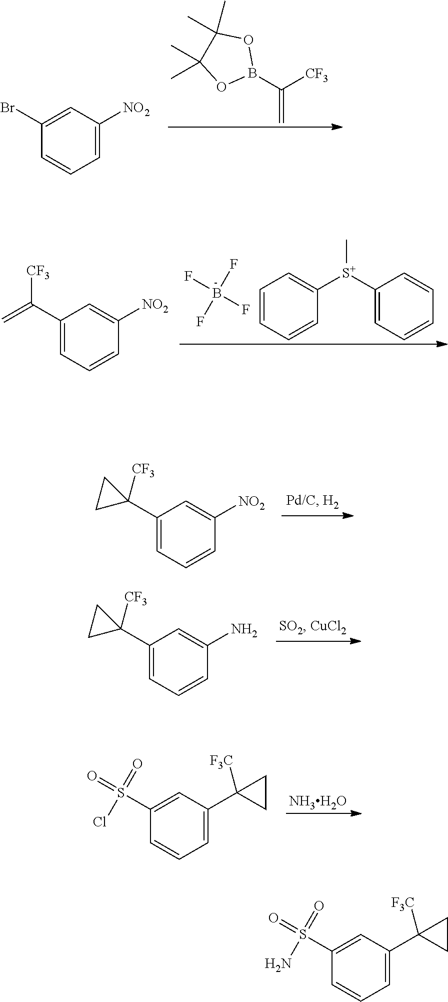

Step 1: 1-nitro-3-(3,3,3-trifluoroprop-1-en-2-yl)benzene: To a solution of 1-bromo-3-nitrobenzene (100 mg, 0.49 mmol) in a mixture of dioxane and water (1.0 mL/0.5 mL) was added 4,4,5,5-tetramethyl-2-(3,3,3-trifluoroprop-1-en-2-yl)-1,3,2-dioxaborolane (218 mg, 0.99 mmol), K₂CO₃ (136 mg, 0.99 mmol), and Pd(PPh₃)₄ (57 mg, 0.49 mmol). The reaction was heated at 110° C. in a microwave reactor overnight. The mixture was diluted with water (20 mL) and extracted with EtOAc (30 mL×3). The combined organic layers were dried over Na₂SO₄, filtered, and concentrated. The residue was purified by prep-TLC (eluent: petroleum ether/EtOAc=30:1) to afford 1-nitro-3-(3,3,3-trifluoroprop-1-en-2-yl)benzene (69 mg, 65%) as a coloress oil. ¹H NMR (400 MHz, Chloroform-d: δ 8.33 (d, J=2.0 Hz, 1H), 8.30-8.24 (m, 1H), 7.83-7.76 (m, 1H), 7.60 (t, J=8.0 Hz, 1H), 6.13 (d, J=1.6 Hz, 1H), 6.01-5.86 (m, 1H).

Step 2: 1-nitro-3-(1-(trifluoromethyl)cyclopropyl)benzene: To a solution of 1-nitro-3-(3,3,3-trifluoroprop-1-en-2-yl)benzene (1.0 g, 4.61 mmol) and methyldiphenylsulfonium tetrafluoroborate (1.7 g, 5.99 mmol) in THF (10 mL) at 0° C. was added NaHMDS (2 M in THF, 4.61 mL, 9.21 mmol). The reaction was stirred at 0° C. for 30 minutes and then allowed to warm to room temperature and stirred for another 2 hours. The reaction was quenched with water (30 mL) and extracted with EtOAc (50 mL×2). The combined organic layers were washed with brine, dried over Na₂SO₄, filtered, and concentrated. The residue was purified by column chromatography on silica gel (eluent: petroleum ether/EtOAc=30:1) to afford 1-nitro-3-(1-(trifluoromethyl)cyclopropyl)benzene (440 mg, 42%) as a yellow oil. ¹H NMR (400 MHz, Chloroform-d): δ 8.32 (t, J=2.0 Hz, 1H), 8.24-8.18 (m, 1H), 7.81 (d, J=7.8 Hz, 1H), 7.54 (t, J=8.0 Hz, 1H), 1.50-1.44 (m, 2H), 1.14-1.06 (m, 2H).

Step 3: 3-(1-(trifluoromethyl)cyclopropyl)aniline: To a solution of 1-nitro-3-(1-(trifluoromethyl)cyclopropyl)benzene (520 mg, 2.25 mmol) in MeOH (2 mL) was added 10% Pd/C (208 mg), and the reaction was stirred under a H₂ atmosphere for 2 hours. The catalyst was removed by filtration through Celite, and the filtrate was concentrated to afford 3-(1-(trifluoromethyl)cyclopropyl)aniline (360 mg, 79%), which was used directly in the next step. LCMS m/z=202.1 [M+H]⁺. ¹H NMR (400 MHz, Chloroform-d): δ 7.12 (t, J=7.8 Hz, 1H), 6.85 (d, J=7.6 Hz, 1H), 6.80 (d, J=2.0 Hz, 1H), 6.69-6.60 (m, 1H), 1.32-1.27 (m, 2H), 1.05-0.97 (m, J=1.8 Hz, 2H).

Step 4: 3-(1-(trifluoromethyl)cyclopropyl)benzenesulfonyl chloride: To a solution of 3-(1-(trifluoromethyl)cyclopropyl)aniline (100 mg, 0.49 mmol) in a mixture of glacial acetic (0.4 mL) and concentrated HCl (0.8 mL) at 0° C. was added a solution of sodium nitrite (36 mg, 0.53 mmol) in water (0.14 mL) dropwise. The reaction was stirred at 0° C. for 30 minutes. A solution of CuCl₂ (28.04 mg, 0.18 mmol) in H₂O (0.1 mL) was added to a cold solution of SO₂ in glacial acid (0.6 mL), and the diazonium salt solution was then added in portions to the cooled SO₂—CuCl₂ mixture. The reaction was stirred for 3 hours and then allowed to warm to room temperature and stirred overnight. The mixture was diluted with water (10 mL) and extracted with EtOAc (20 mL×3). The combined organic layers were washed with brine, dried over Na₂SO₄, filtered, and concentrated to afford 3-(1-(trifluoromethyl)cyclopropyl)benzenesulfonyl chloride (100 mg, 70%), which was used directly in the next step. ¹H NMR (400 MHz, Chloroform-d): δ 8.11 (s, 1H), 8.02 (d, J=8.0 Hz, 1H), 7.85 (d, J=7.8 Hz, 1H), 7.63 (t, J=7.8 Hz, 1H), 1.26 (s, 2H), 1.12 (s, 2H).

Step 5: 3-(1-(trifluoromethyl)cyclopropyl)benzenesulfonamide: A mixture of 3-(1-(trifluoromethyl)cyclopropyl)benzenesulfonyl chloride (100 mg, 0.35 mmol) in ammonium hydroxide (1 mL) was stirred at room temperature for 3 hours and then diluted with water (10 mL) and extracted with EtOAc (20 mL×3). The combined organic layers were washed with brine, dried over Na₂SO₄, filtered, and concentrated to afford 3-(1-(trifluoromethyl)cyclopropyl) benzenesulfonamide (60 mg, 64.5%), which was used directly in the next step. LCMS m/z=264.1 [M−H]⁻. ¹HNMR (400 MHz, Chloroform-d): δ 8.02 (d, J=2.0 Hz, 1H), 7.93-7.88 (m, 1H), 7.70 (d, J=7.6 Hz, 1H), 7.52 (t, J=7.8 Hz, 1H), 1.48-1.40 (m, 2H), 1.13-1.05 (m, 2H).

### Synthesis of Building Block: 7-bromo-5-fluorobenzo[d]thiazole

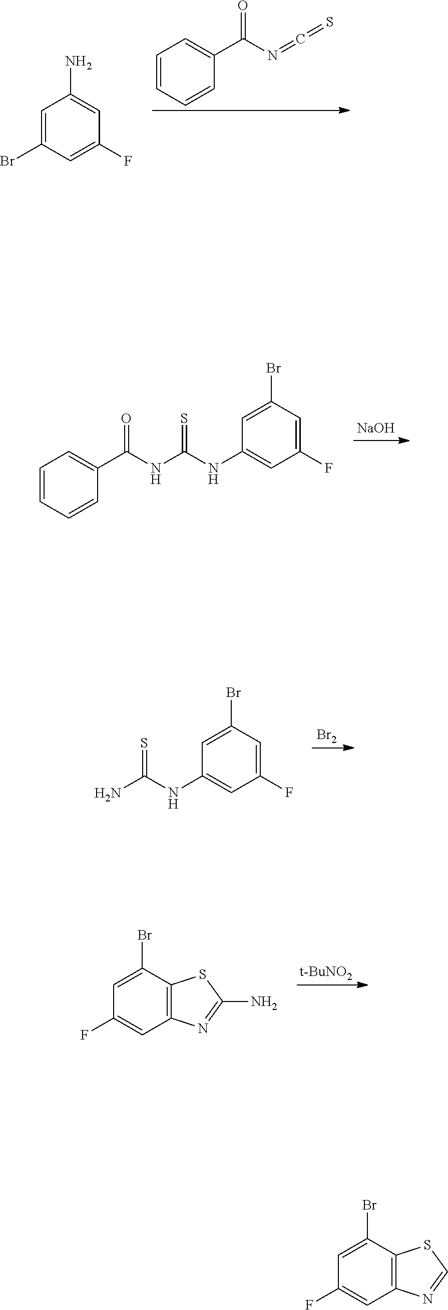

Step 1: N-((3-bromo-5-fluorophenyl)carbamothioyl)benzamide: To a solution of 3-bromo-5-fluoroaniline (500 mg, 2.6 mmol) in acetone (5 mL) was added benzoyl isothiocyanate (431 mg, 2.6 mmol), and the reaction was stirred at room temperature for 30 minutes. The solid precipitate was collected by filtration and washed with acetone to afford N-((3-bromo-5-fluorophenyl)carbamothioyl)benzamide (768 mg, 83%) as a white solid. LCMS m/z=325.7 [M+H]⁺. ¹H NMR (400 MHz, DMSO-d₆): δ 12.59 (s, 1H), 11.72 (s, 1H), 8.00-7.96 (m, 2H), 7.85 (d, 0.1=2.2 Hz, 1H), 7.74 (dt, J=10.6, 2.1 Hz, 1H), 7.70-7.65 (m, 1H), 7.55 (t, 0.1=7.7 Hz, 2H), 7.47 (dt, J=8.3, 2.0 Hz, 1H).

Step 2: 1-(3-bromo-5-fluorophenyl)thiourea: To a solution of N-((3-bromo-5-fluorophenyl)carbamothioyl)benzamide (700 mg, 1.9 mmol) in THF (5 mL) was added 10% NaOH (0.5 mL), and the reaction was stirred at room temperature for 2 hours. The mixture was diluted with water (30 mL) and extracted with EtOAc (50 mL×3). The combined organic layers were washed with brine, dried over Na₂SO₄, filtered, and concentrated to afford 1-(3-bromo-5-fluorophenyl)thiourea (200 mg, 40%) as a white solid. ¹H NMR (400 MHz, CDCl₃): δ 7.30-7.25 (m, 1H), 7.18-7.11 (m, 3H), 4.19 (q, J=7.1 Hz, 2H), 3.62 (s, 2H), 2.52 (ddd, J=11.6, 7.8, 3.4 Hz, 1H), 1.89 (td, J=15.8, 11.5, 4.7 Hz, 4H), 1.81-1.74 (m, 1H), 1.52-1.22 (m, 8H).

Step 3: 7-bromo-5-fluorobenzo[d]thiazol-2-amine: To a solution of 1-(3-bromo-5-fluorophenyl)thiourea (40 mg, 0.16 mmol) in dry CHCl₃ at −60° C. was added Br₂ (51 mg, 0.32 mmol). The reaction was stirred for 10 minutes and then heated at 60° C. for 4 hours. The precipitates that formed were collected by filtration and then redissolved in water. The solution was made basic with aqueous ammonia to pH=10-12. The aqueous layer was extracted with EtOAc (30 mL×3), and the combined organic layers were washed with brine, dried over Na₂SO₄, filtered, and concentrated to afford 7-bromo-5-fluorobenzo[d]thiazol-2-amine e (37 mg, 93%) as a white solid. LCMS m %=246.8 [M+H]⁺. ¹H NMR (400 MHz, CD₃OD): δ 7.07 (d, J=2.3 Hz, 1H), 7.03 (d, J=2.3 Hz, 1H).

Step 4: 7-bromo-5-fluorobenzo[d]thiazole: To a solution of 7-bromo-5-fluorobenzo[d]thiazol-2-amine (37 mg, 0.15 mmol) in dioxane was added t-BuNO₂ (30 mg, 0.3 mmol). The reaction was heated at 85° C. overnight. The mixture was diluted with water (30 mL) and extracted with EtOAc (50 mL×3). The combined organic layers were washed with brine, dried over Na₂SO₄, filtered, and concentrated. The mixture was purified by prep-TLC (eluent: petroleum ether/EtOAc=10:1) to afford 7-bromo-5-fluorobenzo[d]thiazole (21 mg, 62%) as a white solid. LCMS m/z=231.5 [M+H]⁺. ¹H NMR (400 MHz, DMSO-d₆): δ 9.59 (s, 1H), 8.04 (dd, J=9.5, 2.3 Hz, 1H), 7.82 (dd, J=8.7, 2.3 Hz, 1H).

### Synthesis of Building Block: 3-(tetrahydro-2H-pyran-4-yl)benzenesulfonamide

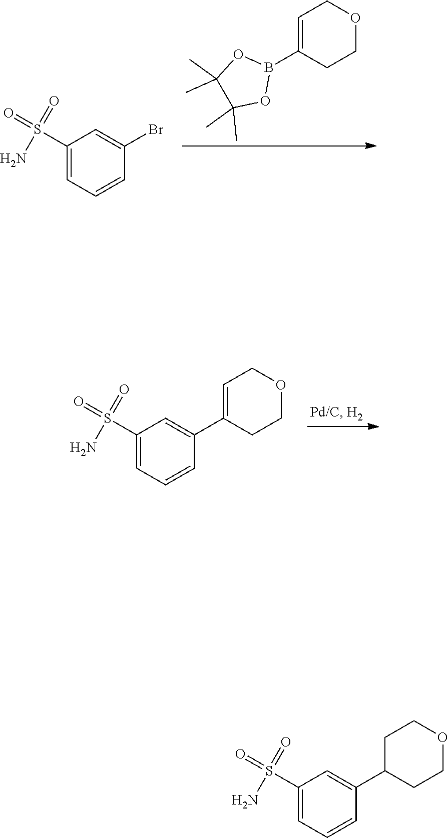 [SMILES: NS(=O)(=O)c1cccc(Br)c1 ; CC1(C)OB(C2=CCOCC2)OC1(C)C ; NS(=O)(=O)c1cccc(C2=CCOCC2)c1 ; NS(=O)(=O)c1cccc(C2CCOCC2)c1]

Step 1: 3-(3,6-dihydro-2H-pyran-4-yl)benzenesulfonamide: To a solution of 3-bromobenzenesulfonamide (1 g, 4.2 mmol) in a mixture of dioxane and water (6 mL/2 mL) was added 2-(3,6-dihydro-2H-pyran-4-yl)-4,4,5,5-tetramethyl-1,3,2-dioxaborolane (983 mg, 4.7 mmol), Pd(PPh₃)₄ (245 mg, 0.2 mmol), and K₂CO₃ (1.17 g, 8.5 mmol). The reaction was heated at 100° C. overnight. The mixture was diluted with water (30 mL) and extracted with EtOAc (50 mL×3). The combined organic layers were washed with brine, dried over Na₂SO₄, filtered, and concentrated. The residue was purified by column chromatography on silica gel (eluent: petroleum ether/EtOAc=5:1) to afford 3-(3,6-dihydro-2H-pyran-4-yl)benzenesulfonamide (900 mg, 90%) as a white solid. ¹H NMR (400 MHz, Methanol-d4): δ 7.96 (s, 1H), 7.79 (d, J=7.8 Hz, 1H), 7.66 (d, J=7.9 Hz, 1H), 7.51 (t, J=7.8 Hz, 1H), 6.33-6.29 (m, 1H), 4.32 (q, J=2.7 Hz, 2H), 3.94 (t, J=5.5 Hz, 2H), 2.59-2.50 (m, 2H).

Step 2: 3-(tetrahydro-2H-pyran-4-yl)benzenesulfonamide: To a solution of 3-(3,6-dihydro-2H-pyran-4-yl)benzenesulfonamide (400 mg, 2.9 mmol) in MeOH (5 mL) was added 10% Pd/C (50 mg). The reaction was stirred at room temperature under H₂ atmosphere for 2 hours. The catalyst was removed by filtration through Celite, and the filtrate was concentrated to afford 3-(tetrahydro-2H-pyran-4-yl)benzenesulfonamide (400 mg, 98%) as a white solid. ¹HNMR (400 MHz, CD₃OD): δ 7.80 (d, 0.1=1.9 Hz, 1H), 7.77-7.72 (m, 1H), 7.51-7.47 (m, 2H), 4.05 (dt, J=11.3, 3.2 Hz, 2H), 3.63-3.52 (m, 2H), 2.96-2.85 (m, 1H), 1.85-1.76 (m, 4H).

### Synthesis of 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoic acid I-54

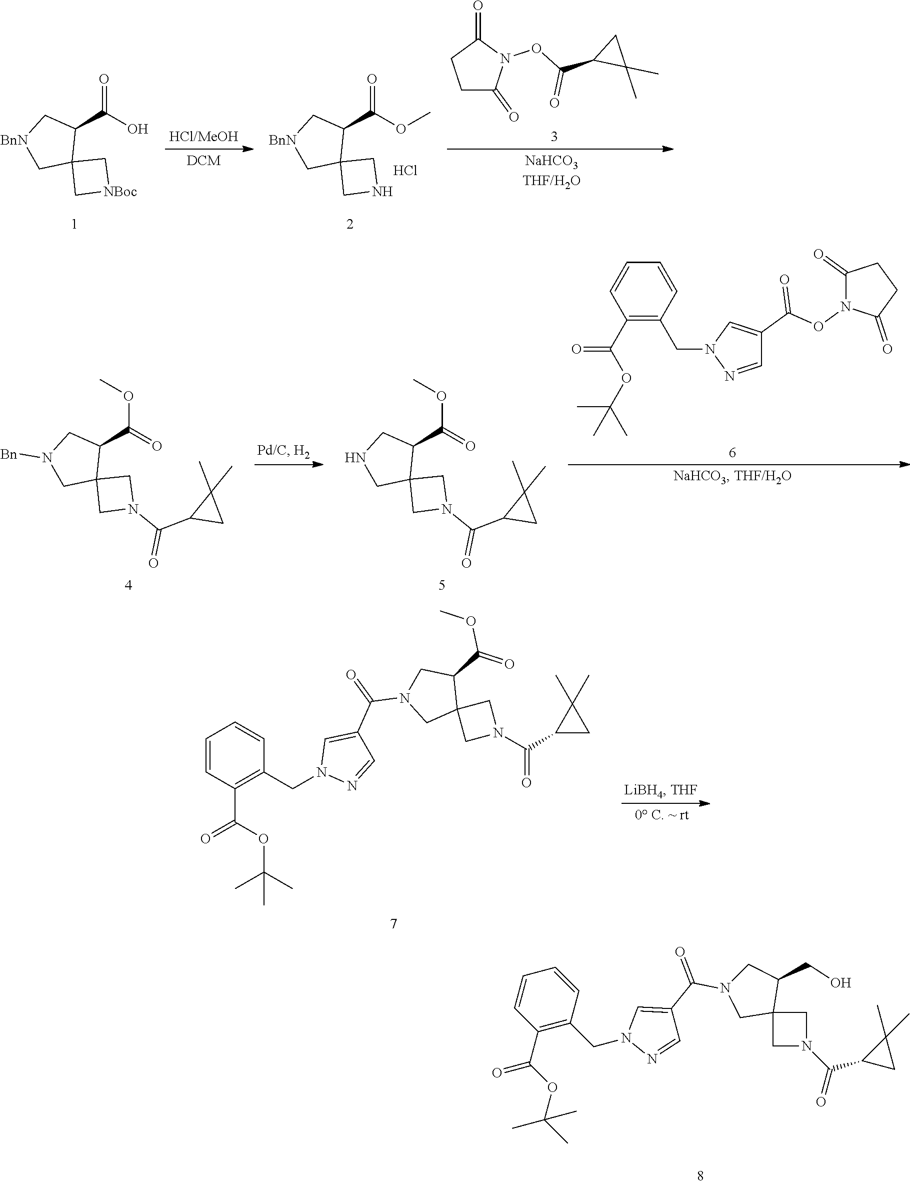

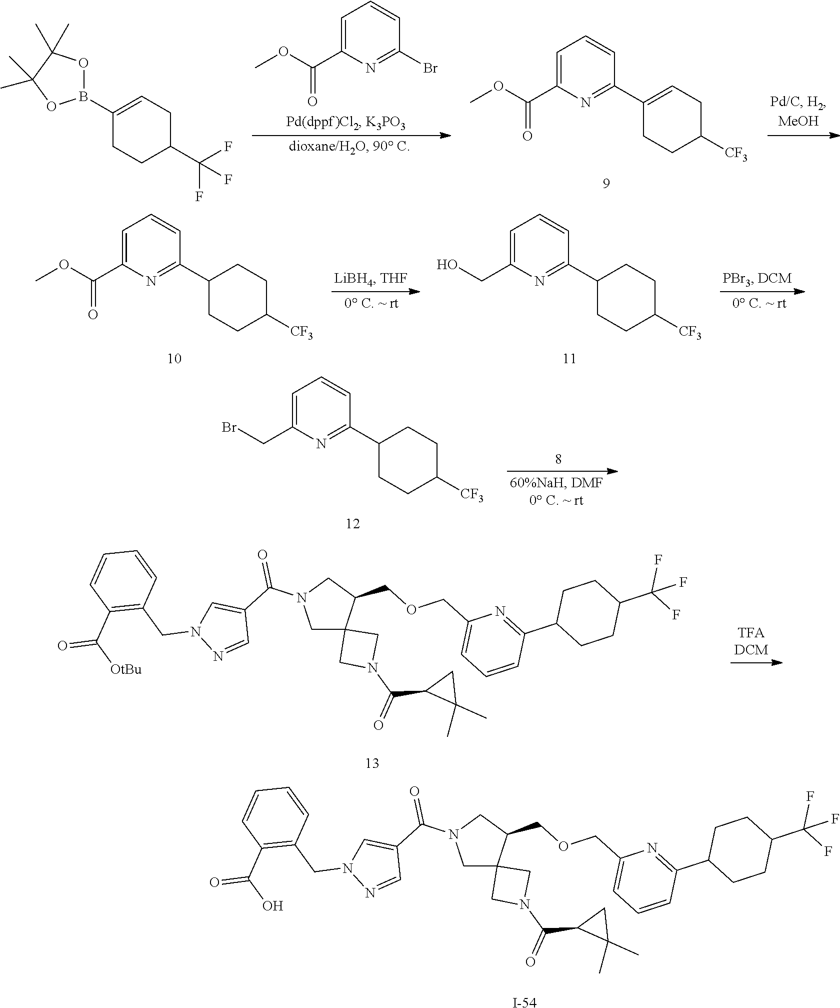

Step 1: Methyl (S)-6-benzyl-2-((S)-2,2-dimethylcyclopropane-1-carbonyl)-2,6-diazaspiro [3.4]octane-8-carboxylate (4): A mixture of (S)-6-benzyl-2-(tert-butoxycarbonyl)-2,6-diazaspiro[3.4]octane-8-carboxylic acid synthesis infra, (1.000 g, 11.20 mmol) and hydrogen chloride (5 mL, 4N in MeOH) in dichloromethane (5 mL) was stirred at room temperature under nitrogen atomsphere for 2 hours. TLC showed the reaction was complete. The mixture was concentrated under reduced pressure to afford (S)-6-benzyl-2-((S)-2,2-dimethylcyclopropane-1-carbonyl)-2,6-diazaspiro[3.4]octane-8-carboxylate (4) as a colorless oil, which was taken up in a mixture of tetrahydrofuran (20 mL) and water (20 mL). Next, sodium bicarbonate (3.640 g, 43.33 mmol) and 2,5-dioxopyrrolidin-1-yl (S)-2,2-dimethylcyclopropane-1-carboxylate (3) (2.36 g, 11.20 mmol) were added. The resulting mixture was stirred at room temperature for 2 hours. The reaction mixture was poured into water (5 mL) and extracted with dichloromethane (40 mL). The combined extracts were dried over anhydrous sodium sulfate and concentrated to give a crude residue, which was purified by silica gel column chromatography using a using a 1% methanol in dichloromethane gradient to afford methyl (S)-6-benzyl-2-((S)-2,2-dimethylcyclopropane-1-carbonyl)-2,6-diazaspiro[3.4]octane-8-carboxylate (4) (0.468 g, 35% of 2 steps) as a colorless solid. MS: [MH]⁺ 357.4.

Step 2: Methyl (S)-2-((S)-2,2-dimethylcyclopropane-1-carbonyl)-2,6-diazaspiro [3.4]octane-8-carboxylate (5): A mixture of methyl (S)-6-benzyl-2-((S)-2,2-dimethylcyclopropane-1-carbonyl)-2,6-diazaspiro[3.4]octane-8-carboxylate (4) (0.468 g, 1.31 mmol) and Pd/C (10%, 0.240 g) in methanol (20 mL) was stirred at room temperature under hydrogen atomsphere for 2 hours. The mixture was filtered, and the filtrate was concentrated to give a crude residue, which was purified by silica gel column chromatography using a 5% methanol in dichloromethane gradient to afford methyl (S)-2-((S)-2,2-dimethylcyclopropane-1-carbonyl)-2,6-diazaspiro[3.4]octane-8-carboxylate (5) (0.314 g, 90% yield) as a yellow solid. MS: [MH]⁺ 267.4.

Step 3: Methyl 6-(4-(trifluoromethyl)cyclohex-1-en-1-yl)picolinate (9): To a stirred solution of methyl 6-bromopicolinate (1.200 g, 5.60 mmol), 4,4,5,5-tetramethyl-2-(4-(trifluoromethyl)cyclohex-1-en-1-yl)-1,3,2-dioxaborolane (1.700 g, 6.20 mmol) and potassium phosphate (2.400 g, 11.20 mmol) in 1,4-dioxane (12 mL) and water (3 mL) was added 1,1′-bis(diphenylphosphino)ferrocene palladium(II)dichloride (0.406 g, 0.56 mmol) at room temperature under nitrogen atmosphere. The resulting mixture was stirred at 90° C. under nitrogen atmosphere overnight. The reaction mixture was cooled to room temperature and then partitioned between ethyl acetate (20 mL) and water (10 mL). The organic layer was collected, and the aqueous layer was extracted with ethyl acetate (10 mL×2). The combined organic layers were washed with brine (25 mL), dried over anhydrous sodium sulfate, and concentrated under reduced pressure to give a crude residue, which was purified by silica gel flash chromatography using a 5% ethyl acetate in hexane gradient to afford methyl 6-(4-(trifluoromethyl)cyclohex-1-en-1-yl)picolinate (9) (1.500 g, 94% yield) as white solid. MS: [MH]⁺ 286.10.

Step 4: Methyl 6-(4-(trifluoromethyl)cyclohexyl)picolinate (10): A mixture of methyl 6-(4-(trifluoromethyl)cyclohex-1-en-1-yl)picolinate (9) (1.500 g, 5.30 mmol) and palladium on carbon (10%, 150 mg) in methanol (25 mL) was stirred at room temperature under hydrogen atmosphere (hydrogen balloon) overnight. Palladium on carbon was removed through filtration and washed with methanol (10 mL×2). The combined filtrate was concentrated under reduced pressure to give a crude residue, which was purified by silica gel flash chromatography using a 10% ethyl acetate in hexane gradient to afford methyl 6-(4-(trifluoromethyl)cyclohexyl)picolinate (10) (1.5 g, 85% yield) as a colorless oil. MS: [MH]288.20.

Step 5: Trans-(6-(4-(trifluoromethyl)cyclohexyl)pyridin-2-yl)methanol (11): To a solution of methyl 6-(4-(trifluoromethyl)cyclohexyl)picolinate (10) (1.4 g, 5.0 mmol) in tetrahydrofuran (20 mL) was added lithium borohydride (218.6 mg, 10.0 mmol) at 0° C. The mixture was allowed to warm up to room temperature and stirred for 2 hours under nitrogen atmosphere. The reaction mixture was quenched with saturated aqueous ammonium chloride solution and partitioned between ethyl acetate (15 mL) and water (5 mL). The organic layer was collected, and the aqueous layer was extracted with ethyl acetate (10 mL×2). The combined organic layers were washed with brine (20 mL), dried over anhydrous sodium sulfate, and concentrated under reduced pressure to give a crude residue, which was purified by silica gel flash chromatography using a 12.5% ethyl acetate in hexane gradient to afford (6-(4-(trifluoromethyl)cyclohexyl)pyridin-2-yl)methanol (11) (0.850 g 65% yield) as a white solid. ¹HNMR (400 MHz, DMSO-d6): δ 7.72 (t, J=7.6 Hz, 1H), 7.28 (d, J=7.6 Hz, 1H), 7.18 (d, J=7.6 Hz, 1H), 5.32 (t, J=6.0 Hz, 1H), 4.52 (d, J=5.6 Hz, 2H), 2.98-2.89 (m, 1H), 2.48-2.36 (m, 1H), 2.16-2.04 (m, 2H), 1.81-1.60 (m, 6H). MS: [MH]⁺ 260.20.

Step 6: 2-(Bromomethyl)-6-(4-(trifluoromethyl)cyclohexyl)pyridine (12): To a solution of (6-(4-(trifluoromethyl)cyclohexyl)pyridin-2-yl)methanol (11) (0.760 g, 2.90 mmol) in dichloromethane (10 mL) at 0° C. was added phosphorus tribromide (1.59 g, 5.90 mmol). The mixture was allowed to warm up to room temperature and stirred at room temperature under nitrogen atmosphere for 1 hour. The reaction mixture was neutralized to pH=8 with saturated aqueous sodium bicarbonate solution and extracted with dichloromethane (6 mL×2). The combined organic layers were washed with brine (10 mL), dried over anhydrous sodium sulfate, and concentrated under reduced pressure to give a crude residue, which was purified by silica gel flash chromatography using a 10% ethyl acetate in hexane gradient to afford 2-(bromomethyl)-6-(4-(trifluoromethyl)cyclohexyl)pyridine (12) (0.880 g, 91% yield) as a white solid. MS: [MH]⁺ 324.05.

Step 7: Methyl (S)-6-(1-(2-(tert-butoxycarbonyl)benzyl)-1H-pyrazole-4-carbonyl)-2-((S)-2,2-dimethylcyclopropane-1-carbonyl)-2,6-diazaspiro[3.4]octane-8-carboxylate (7): To a solution of methyl (S)-2-((S)-2,2-dimethylcyclopropane-1-carbonyl)-2,6-diazaspiro[3.4]octane-8-carboxylate (5) (0.610 g, 2.30 mmol) in tetrahydrofuran (5 mL) and water (5 mL) was added sodium bicarbonate (0.962 g, 11.50 mmol). The mixture was stirred for 15 minutes, followed by the addition of 2,5-dioxopyrrolidin-1-yl1-(2-(tert-butoxycarbonyl)benzyl)-1H-pyrazole-4-carboxylate (6) (1.20 g, 3.00 mmol). The resulting mixture was stirred at room temperature for 3 hours, and extracted with ethyl acetate (15 mL×2). The combined organic layers were washed with brine (20 mL), dried over sodium sulfate, and concentrated under reduced pressure to give a crude residue, which was purified by silica gel flash chromatography (eluted with 5% methanol in dichloromethane gradient) to afford methyl (S)-6-(1-(2-(tert-butoxycarbonyl)benzyl)-1H-pyrazole-4-carbonyl)-2-((S)-2,2-dimethylcyclopropane-1-carbonyl)-2,6-diazaspiro[3.4]octane-8-carboxylate (7) (0.465 g, 37% yield) as an off-white solid. MS: [MH]⁺ 551.10.

Step 8: tert-Butyl 2-((4-((S)-2-((S)-2,2-dimethylcyclopropane-1-carbonyl)-8-(hydroxymethyl)-2,6-diazaspiro[3.4]octane-6-carbonyl)-1H-pyrazol-1-yl)methyl)benzoate (8): To a solution of methyl (S)-6-(1-(2-(tert-butoxycarbonyl)benzyl)-1H-pyrazole-4-carbonyl)-2-((S)-2,2-dimethylcyclopropane-1-carbonyl)-2,6-diazaspiro[3.4]octane-8-carboxylate (7) (0.465 g, 0.85 mmol) in tetrahydrofuran (10 mL) at 0° C. was added lithium borohydride (0.037 mg, 1.70 mmol). The mixture was allowed to warm up to room temperature and stirred under nitrogen atmosphere for 2 hours. The resulting mixture was quenched with saturated aqueous ammonium chloride solution and partitioned between ethyl acetate (15 mL) and water (5 mL). The organic layer was collected, and the aqueous layer was extracted with ethyl acetate (10 mL×2). The combined organic layers were washed with brine (20 mL), dried over anhydrous sodium sulfate, and concentrated under reduced pressure to give a crude residue, which was purified by silica gel flash chromatography using a 5%˜10% methanol in dichloromethane gradient to afford tert-butyl 2-((4-((S)-2-((S)-2,2-dimethylcyclopropane-1-carbonyl)-8-(hydroxymethyl)-2,6-diazaspiro[3.4]octane-6-carbonyl)-1H-pyrazol-1-yl)methyl)benzoate (8) (0.280 g; 61% yield) as a white solid. MS: [MH]⁺ 523.35.

Step 9: tert-Butyl 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoate (13): To a solution of tert-butyl 2-((4-((S)-2-((S)-2,2-dimethylcyclopropane-1-carbonyl)-8-(hydroxymethyl)-2,6-diazaspiro[3.4]octane-6-carbonyl)-1H-pyrazol-1-yl)methyl)benzoate (8) (0.260 g, 0.50 mmol) in N,N-dimethylformamide (5 mL) at 0-5° C. was added sodium hydride (60% in mineral oil) (24 mg, 1.0 mmol). The resulting mixture was stirred at room temperature for 30 minutes. Next, 2-(bromomethyl)-6-(4-(trifluoromethyl)cyclohexyl)pyridine (12) (168.3 mg, 0.52 mmol) was added to the mixture. The resulting mixture was stirred at room temperature under nitrogen atmosphere for 2 hours. The reaction mixture was then quenched with water and partitioned between ethyl acetate (20 mL) and water (7 mL). The organic phase was washed with water (10 mL×2) and brine (10 mL), dried over anhydrous sodium sulfate, and concentrated under reduced pressure to give a crude residue, which was purified by preparative TLC using a 3.3% methanol in dichloromethane gradient to afford tert-butyl 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoate (13) (0.092 g, 22% yield) as a white solid. MS: [MH]⁺ 764.70.

Step 10: 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoic acid (I-51): A mixture of tert-butyl 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoate (13) (0.092 g, 0.12 mmol) in 2,2,2-trifluoroacetic acid/dichloromethane (2 mL, 1:1) was stirred at 40° C. for 2 hours. The reaction mixture was concentrated under reduced pressure to give a crude residue, which was purified by preparative TLC using a 10% methanol in dichloromethane gradient to afford 2-((4-((S)-2-((S)-2,2-dimethylcyclopropane-1-carbonyl)-8-(((6-(4-(trifluoromethyl)cyclohexyl)pyridin-2-yl)methoxy)methyl)-2,6-diazaspiro[3.4]octane-6-carbonyl)-1H-pyrazol-1-yl)methyl)benzoic acid (I-51) (0.073 g, 85% yield) as a white solid. ¹HNMR (400 MHz, CD₃OD): δ 8.22 (d, J=7.2 Hz, 1H), 8.05 (d, J=7.6 Hz, 1H), 7.93 (d, J 8.4 Hz, 1H), 7.71-7.63 (m, 1H), 7.54-7.46 (m, 1H), 7.46-7.38 (m, 1H), 7.29-7.18 (m, 2H), 6.99 (t, J=8.0 Hz, 1H), 5.82 (s, 2H), 4.65-4.56 (m, 2H), 4.51-3.55 (m, 11H), 2.99-2.89 (m, 1H), 2.80-2.64 (m, 1H), 2.39-2.25 (m, 1H), 2.15-2.03 (m, 2H), 1.83-1.78 (m, 4H), 1.44-1.25 (m, 2H), 1.19-1.03 (m, 7H), 0.78-0.70 (m, 1H). MS: [MH]⁺ 708.80.

The following compound was prepared in a manner analogous to the procedure described above for 2-((4-((S)-2-((S)-2,2-dimethylcyclopropane-1-carbonyl)-8-(((6-(4-(trifluoromethyl) cyclohexyl)pyridin-2-yl)methoxy)methyl)-2,6-diazaspiro[3.4]octane-6-carbonyl)-1H-pyrazol-1-yl)methyl)benzoic acid (I-54).

(S)-2-(4,4-difluorocyclohexyl)-6-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl) benzoic acid I-51 (0.54 g, 58%) as a white solid. ¹HNMR (400 MHz, CD₃OD): δ 8.29-8.22 (m, 1H), 7.93 (d, J=6.8 Hz, 1H), 7.36-7.30 (m, 4H), 7.25-7.21 (m, 1H), 7.12-7.07 (m, 2H), 5.38 (s, 2H), 4.61 (s, 2H), 4.42-3.50 (m, 10H), 2.91-2.85 (m, 1H), 2.74-2.61 (m, 1H), 2.16-2.09 (m, 2H), 1.95-1.78 (m, 6H), 1.20-1.17 (m, 4H). MS: [MH]⁺ 733.15.

### Synthesis of ((R)-2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octan-6-yl)(thiazol-5-yl) methanone I-64

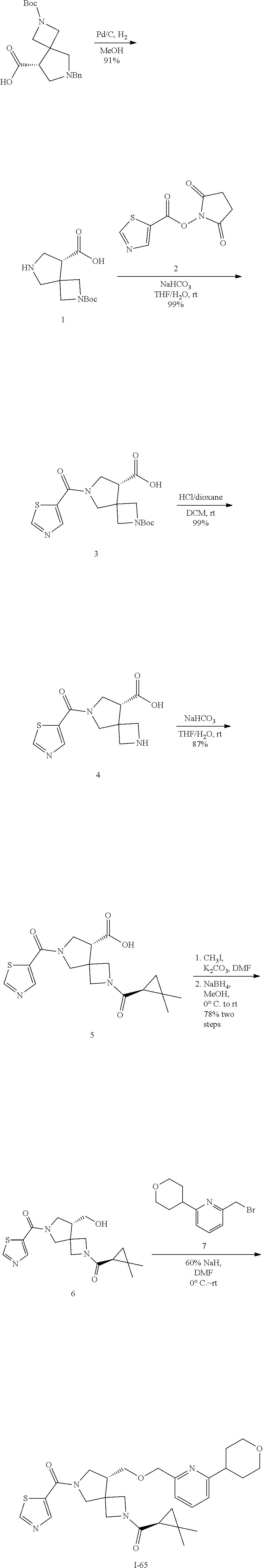

Step 1: (R)-2-(tert-butoxycarbonyl)-2,6-diazaspiro[3.4]octane-8-carboxylic acid (1): A mixture of 6-benzyl-2-(tert-butoxycarbonyl)-2,6-diazaspiro[3.4]octane-8-carboxylic acid (1.600 g, 4.62 mmol) and palladium on carbon (5%, 0.160 g) in methanol-water (30 mL/10 mL) was stirred at 40° C. under hydrogen atmosphere (hydrogen balloon) overnight. The mixture was cooled to room temperature and additional water (20 mL) was added. The resulting mixture was stirred at room temperature for an additional 1 hour. Palladium on carbon was removed through filtration and washed with methanol-water (1.1 v/v, 30 mL x2). The combined filtrate was concentrated under reduced pressure to afford (R)-2-(tert-butoxycarbonyl)-2,6-diazaspiro[3.4]octane-8-carboxylic acid (1) (1.000 g, 91% yield) as a white solid, which was used in the next step without further purification.

Step 2: (R)-2-(tert-butoxycarbonyl)-6-(thiazole-5-carbonyl)-2,6-diazaspiro[3.4]octane-8-carboxylic acid (3): To a mixture of (R)-2-(tert-butoxycarbonyl)-2,6-diazaspiro[3.4]octane-8-carboxylic acid (1) (1.000 g, 4.14 mmol) and sodium bicarbonate (0.348 g, 4.14 mmol) in tetrahydrofuran-water (8 mL/8 mL) at room temperature was added a solution of 2,5-dioxopyrrolidin-1-yl thiazole-5-carboxylate (2) (936.2 mg, 4.14 mmol) in tetrahydrofuran (4 mL). The resulting mixture was stirred at room temperature for 30 minutes. The reaction mixture was extracted with ethyl acetate (10 mL×2) to remove some impurities. The aqueous layer was collected, acidified with diluted hydrochloric acid (1N) to a pH of 3-4, and extracted with dichloromethane (15 mL×3). The combined organic layers were washed with brine (20 mL), dried over sodium sulfate, and concentrated under reduced pressure to afford (R)-2-(tert-butoxycarbonyl)-6-(thiazole-5-carbonyl)-2,6-diazaspiro[3.4]octane-8-carboxylic acid (3) (1.400 g, yield 100%) as an off-white solid. 1HNMR (400 MHz, CDCl₃): δ 8.95 (s, 1H), 8.27 (s, 1H), 4.11-3.75 (m, 8H), 3.28-3.20 (m, 1H), 1.47-1.40 (m, 9H). MS: [MH]⁺ 735.65.

Step 3: (R)-6-(thiazole-5-carbonyl)-2,6-diazaspiro[3.4]octane-8-carboxylic acid hydrochloride (4): A mixture of (R)-2-(tert-butoxycarbonyl)-6-(thiazole-5-carbonyl)-2,6-diazaspiro[3.4]octane-8-carboxylic acid (3) (1.400 g, 1.90 mmol) and hydrogen chloride 1,4-dioxane solution (4 M, 5 mL) in dichloromethane (10 mL) was stirred at room temperature for 1 hour. The volatiles were removed under reduced pressure to afford (R)-6-(thiazole-5-carbonyl)-2,6-diazaspiro[3.4]octane-8-carboxylic acid hydrochloride (4) (crude 1.5 g) as a white solid, which was used in the next step without further purification. MS: [MH]⁺ 267.90.

Step 4: (R)-2-((S)-2,2-dimethylcyclopropane-1-carbonyl)-6-(thiazole-5-carbonyl)-2,6-diazaspiro[3.4]octane-8-carboxylic acid (5): To a mixture of (R)-6-(thiazole-5-carbonyl)-2,6-diazaspiro[3.4]octane-8-carboxylic acid hydrochloride (4) (1.5 g crude) and sodium bicarbonate (1.300 g, 15.80 mmol) in tetrahydrofuran-water (8 mL/8.0 mL) at room temperature was added a mixture of 2,5-dioxopyrrolidin-1-yl (S)-2,2-dimethylcyclopropane-1-carboxylate (0.834 g, 3.95 mmol) in tetrahydrofuran (4 mL). The resulting mixture was stirred at room temperature for 30 minutes. The reaction mixture was extracted with ethyl acetate (10 mL×2) to remove impurities. The aqueous layer was collected, acidified with diluted hydrochloric acid (1N) to a pH of 3-4, and extracted with dichloromethane (15 mL×3). The combined organic layers were washed with brine (20 mL), dried over sodium sulfate, and concentrated under reduced pressure to afford (R)-2-((S)-2,2-dimethylcyclopropane-1-carbonyl)-6-(thiazole-5-carbonyl)-2,6-diazaspiro[3.4]octane-8-carboxylic acid (5) (1.200 g, yield 90%) as a white solid. MS: [MH]⁺ 364.40.

Step 5: (2-((S)-2,2-dimethylcyclopropane-1-carbonyl)-8-(hydroxymethyl)-2,6-diazaspiro[3.4]octan-6-yl)(thiazol-5-yl)methanone (6): To a suspension of (R)-2-((S)-2,2-dimethylcyclopropane-1-carbonyl)-6-(thiazole-5-carbonyl)-2,6-diazaspiro[3.4]octane-8-carboxylic acid (5) (0.500 g, 1.38 mmol) and K₂CO₃ (0.381 g, 2.76 mmol) in DMF (8 mL) was added iodomethane (0.586 g, 4.13 mmol). The reaction mixture was stirred at room temperature for 2 hours. The mixture was concentrated, and the residue was purified by silica gel flash chromatography using a 5% methanol in dichloromethane gradient to afford a methyl ester (0.500 g) as a yellow oil. To a solution of the methyl ester (0.495 g, 1.31 mmol) in methanol (8 mL) at 0° C. was added NaBH₄ (0.199 g, 5.25 mmol) in portions. The resulting mixture was stirred at room temperature overnight. The reaction mixture was quenched with diluted hydrochloric acid (2N), adjusted to pH=7, and concentrated to give a residue, which was purified by silica gel flash chromatography using a 5% methanol in dichloromethane gradient to afford (2-((S)-2,2-dimethylcyclopropane-1-carbonyl)-8-(hydroxymethyl)-2,6-diazaspiro[3.4]octan-6-yl)(thiazol-5-yl)methanone (6) (0.377 g, yield 82%) as a white solid. MS: [M]H]⁺ 350.55. Step 6: ((R)-2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octan-6-yl)(thiazol-5-yl)methanone (I-64): To a solution of (2-((S)-2,2-dimethylcyclopropane-1-carbonyl)-8-(hydroxymethyl)-2,6-diazaspiro[3.4]octan-6-yl)(thiazol-5-yl)methanone (6) (0.072 g, 0.21 mmol) in N,N-dimethylformamide (3 mL) at 0-5° C. was added sodium hydride (60% in mineral oil, 0.033 g, 0.83 mmol). The resulting mixture was stirred at room temperature for 30 minutes. 2-(Bromomethyl)-6-(tetrahydro-2H-pyran-4-yl)pyridine (7) (0.053 g, 0.21 mmol) was added, and the resulting mixture was stirred at room temperature for 2 hours under nitrogen atmosphere. The reaction mixture was then poured into water (5 mL) and extracted with ethyl acetate (15 mL). The organic layer was collected, washed with water (8 mL×2) and brine (10 mL), dried over anhydrous sodium sulfate, and concentrated under reduced pressure to give a crude residue, which was purified by preparative TLC using a 5% methanol in dichloromethane gradient to afford ((R)-2-((S)-2,2-dimethylcyclopropane-1-carbonyl)-8-(((6-(tetrahydro-2H-pyran-4-yl)pyridin-2-yl)methoxy) methyl)-2,6-diazaspiro[3.4]octan-6-yl)(thiazol-5-yl)methanone (I-64) (0.055 g, 51% yield) as an off-white solid. ¹HNMR (400 MHz, CD₃OD): δ 9.15 (s, 1H), 8.38-8.34 (m, 1H), 7.76-7.69 (m, 1H), 7.33-7.24 (m, 1H), 7.21 (d, J=7.6 Hz, 1H), 4.65-4.55 (m, 2H), 4.31-3.60 (m, 12H), 3.60-3.52 (m, 2H), 3.00-2.90 (m, 1H), 2.82-2.69 (m, 1H), 1.93-1.77 (m, 4H), 1.45-1.37 (m, 1H), 1.21-1.01 (m, 7H), 0.79-0.73 (m, 1H). MS: [MH]⁺ 526.05.

### Synthesis of 6-(5-fluorobenzo[d]thiazol-7-yl)-N-((3-(tetrahydro-2H-pyran-4-yl)phenyl)sulfonyl)-2-(1-(trifluoromethyl)cyclopropane-1-carbonyl)-2,6-diazaspiro[3.4]octane-8-carboxamide (I-53)

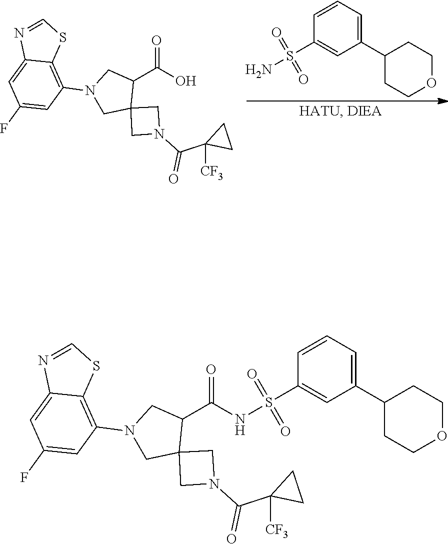 [SMILES: O=C(O)C1CN(c2cc(F)cc3ncsc23)CC12CN(C(=O)C1(C(F)(F)F)CC1)C2 ; NS(=O)(=O)c1cccc(C2CCOCC2)c1 ; O=C(NS(=O)(=O)c1cccc(C2CCOCC2)c1)C1CN(c2cc(F)cc3ncsc23)CC12CN(C(=O)C1(C(F)(F)F)CC1)C2]

6-(5-fluorobenzo[d]thiazol-7-yl)-N-((3-(tetrahydro-2H-pyran-4-yl)phenyl)sulfonyl)-2-(1-(trifluoromethyl)cyclopropane-1-carbonyl)-2,6-diazaspiro[3.4]octane-8-carboxamide (I-53): To a solution of 6-(5-fluorobenzo[d]thiazol-7-yl)-2-(1-(trifluoromethyl)cyclopropane-1-carbonyl)-2,6-diazaspiro[3.4]octane-8-carboxylic acid (50 mg, 0.11 mmol) in DMF (2.0 mL) was added HATU (42 mg, 0.11 mmol) and DIPEA (44 mg, 0.33 mmol) and the mixture stirred at room temperature for 15 min. 3-(tetrahydro-2H-pyran-4-yl)benzenesulfonamide (28 mg, 0.11 mmol) was added and stirring continued overnight. The mixture was diluted with water (30 mL) and extracted with EtOAc (50 mL×3). The combined organic layers were washed with brine, dried over Na₂SO₄, filtered and concentrated. The residue obtained was purified by prep-HPLC to afford 6-(5-fluorobenzo[d]thiazol-7-yl)-N-((3-(tetrahydro-2H-pyran-4-yl)phenyl)sulfonyl)-2-(1-(trifluoromethyl)cyclopropane-1-carbonyl)-2,6-diazaspiro[3.4]octane-8-carboxamide (4 mg, 5%) as a white solid. LCMS m/z=667.2 [M+H]⁺; ¹H NMR (400 MHz, DMSO-d6) δ 9.35 (s, 1H), 7.70 (s, 2H), 7.53-7.41 (m, 2H), 7.17 (dd, J=9.2, 2.2 Hz, 1H), 6.32 (dd, J=12.2, 2.2 Hz, 1H), 4.19 (m, 1H), 3.90 (m, 3H), 3.82 (m, 3H), 3.67 (m, 3H), 3.43-3.35 (m, 3H), 2.80 (s, 1H), 1.66-1.54 (m, 4H), 1.23-1.12 (m, 4H).

### Synthesis of methyl 3-hydroxy-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate (I-55)

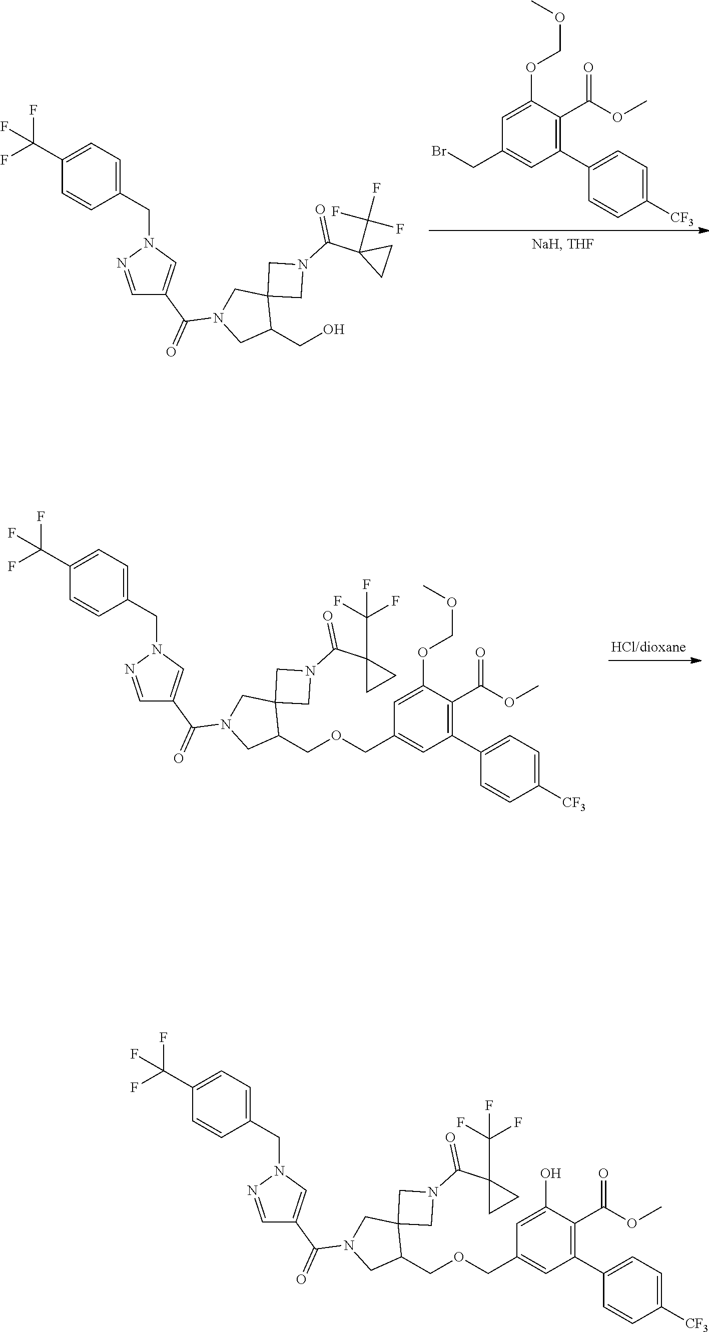 [SMILES: O=C(c1cnn(Cc2ccc(C(F)(F)F)cc2)c1)N1CC(CO)C2(C1)CN(C(=O)C1(C(F)(F)F)CC1)C2 ; COCOc1cc(CBr)cc(-c2ccc(C(F)(F)F)cc2)c1C(=O)OC ; COCOc1cc(COCC2CN(C(=O)c3cnn(Cc4ccc(C(F)(F)F)cc4)c3)CC23CN(C(=O)C2(C(F)(F)F)CC2)C3)cc(-c2ccc(C(F)(F)F)cc2)c1C(=O)OC ; COC(=O)c1c(O)cc(COCC2CN(C(=O)c3cnn(Cc4ccc(C(F)(F)F)cc4)c3)CC23CN(C(=O)C2(C(F)(F)F)CC2)C3)cc1-c1ccc(C(F)(F)F)cc1]

Step 1: methyl 3-(methoxymethoxy)-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate: To a solution of methyl 5-(bromomethyl)-3-(methoxymethoxy)-4′-(trifluoromethyl)-[1,1′-biphenyl]-2-carboxylate (300 mg, 0.69 mmol) in THF (8 mL) 0° C. was added (8-(hydroxymethyl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (367 mg, 0.69 mmol) and NaH (42 mg, 1.04 mmol) and the reaction was stirred at room temperature overnight. The mixture was diluted with water (50 mL) and extracted with EtOAc (20 mL×3). The combined organic layers were washed with brine, dried over Na₂SO₄, filtered and concentrated. The residue obtained was purified by prep-TLC (eluent: DCM:MeOH=50:1) to afford methyl 3-(methoxymethoxy)-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate (380 mg, 62%) as a yellow oil. LCMS m/z=883.4 [M+H]⁺; ¹H NMR (400 MHz, DMSO-d₆) δ 8.38 (d, J=4.8 Hz, 1H), 7.86-7.76 (m, 3H), 7.71 (d, J=8.0 Hz, 2H), 7.54 (d, J=7.8 Hz, 2H), 7.43 (t, J=6.8 Hz, 2H), 7.21 (s, 1H), 7.04 (s, 1H), 5.47 (d, J=6.0 Hz, 2H), 5.26 (d, J=4.0 Hz, 2H), 4.56 (s, 2H), 4.18 (m, 2H), 3.97 (m, 2H), 3.85 (d, J=8.4 Hz, 1H), 3.71 (m, 3H), 3.60 (s, 3H), 3.41 (m, 1H), 3.37 (d, J=6.4 Hz, 3H), 2.72-2.56 (m, 1H), 1.10 (m, 4H).

Step 2: methyl 3-hydroxy-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate: To a solution of methyl 3-(methoxymethoxy)-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate (30 mg, 0.03 mmol) in DCM (2 mL) was added a solution of HCl in dioxane (4 M, 1 mL). The reaction was stirred at room temperature for 1 h then the solvent was removed under vacuum. The residue obtained was purified by prep-HPLC to afford methyl 3-hydroxy-4′-(trifluoromethyl)-5-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-[1,1′-biphenyl]-2-carboxylate (10 mg, 35%) as a white solid. LCMS m/z=839.3 [M+H]⁺; ¹H NMR (400 MHz, Methanol-d4) δ 8.24 (d, J=9.4 Hz, 1H), 7.92 (d, J=5.6 Hz, 1H), 7.66 (t, J=9.6 Hz, 4H), 7.43 (m, 4H), 6.94 (d, J=6.0 Hz, 1H), 6.81 (d, J=6.6 Hz, 1H), 5.47 (t, J=4.6 Hz, 2H), 4.55 (m, 4H), 4.10-3.90 (m, 3H), 3.81 (m, 1H), 3.73 (m, 2H), 3.70-3.56 (m, 2H), 3.55 (s, 3H), 3.35 (m, 1H), 1.17 (m, 4H).

### Synthesis of (2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octan-6-yl)(thiazol-5-yl)methanone (1-49)

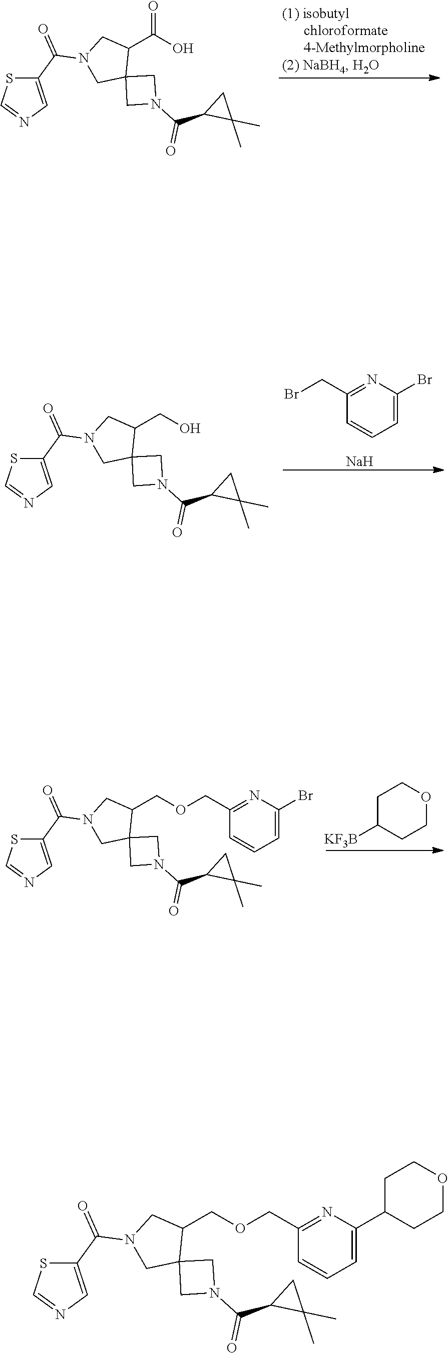

Step 1: (2-((S)-2,2-dimethylcyclopropane-1-carbonyl)-8-(hydroxymethyl)-2,6-diazaspiro[3.4]octan-6-yl)(thiazol-5-yl)methanone: To a solution of 2-((S)-2,2-dimdethylcyclopropane-1-carbonyl)-6-(thiazole-5-carbonyl)-2,6-diazaspiro[3.4]octane-8-carboxylic acid (1.0 g, 2.75 mmol) in anhydrous THF (5.0 mL) at 0° C. was added 4-Methylmorpholine (373 mg, 3.69 mmol) and isobutyl chloroformate (0.5 mL, 3.69 mmol). The reaction was stirred 30 min then a solution of NaBH₄ (312 mg, 8.25 mmol) in water (4.0 mL) was added dropwise. The reaction was stirred at room temperature for 1 h then was diluted with water and extracted with EtOAc (30 mL×2). The combine organic layers were washed with brine, dried over Na₂SO₄, filtered and concentrated. The residue obtained was purified by column chromatography on silica gel (eluent: DCM/MeOH=20/1) to afford (2-((S)-2,2-dimethylcyclopropane-1-carbonyl)-8-(hydroxymethyl)-2,6-diazaspiro[3.4]octan-6-yl)(thiazol-5-yl)methanone (540 mg, 56%) as a white solid. LCMS m/z=350.5 [M+H]⁺; ¹H NMR (400 MHz, CD₃OD) 10.05 (d, J=2.2 Hz, 1H), 9.18 (d, J=11.8 Hz, 1H), 5.31-4.21 (m, 11H), 2.15-2.10 (m, 1H), 1.97-1.82 (m, 6H), 1.74-1.62 (m, 1H), 1.52-1.34 (m, 1H).

Step 2: (8-(((6-bromopyridin-2-yl)methoxy)methyl)-2-((S)-2,2-dimethylcyclopropane-1-carbonyl)-2,6-diazaspiro[3.4]octan-6-yl)(thiazol-5-yl)methanone: To a solution of (2-(S)-2,2-dimethylcyclopropane-1-carbonyl)-8-(hydroxymethyl)-2,6-diazaspiro[3.4]octan-6-yl)(thiazol-5-yl)methanone (200 mg, 0.57 mmol) THF (8 mL) at 0° C. was added NaH (30 mg, 0.74nmmol) and the mixture stirred for 30 min. 2-bromo-6-(bromomethyl)pyridine (158 mg, 0.63 mmol) was added and the reaction allowed to warm to room temperature and stirred overnight. The reaction was diluted with water and extracted with EtOAc (20 mL×2). The combine organic layers were washed with brine, dried over Na₂SO₄, filtered and concentrated. The residue obtained was purified by column chromatography on silica gel (eluent: DCM/MeOH=20/1) to afford (8-(((6-bromopyridin-2-yl)methoxy)methyl)-2-((S)-2,2-dimethylcyclopropane-1-carbonyl)-2,6-diazaspiro[3.4]octan-6-yl)(thiazol-5-yl)methanone (130 mg, 40%) as a white solid. LCMS m/z=519.5 [M+H]⁺; ¹H NMR (400 MHz, CD₃OD) δ 9.18 (s, 1H), 8.39 (d, J=3.7 Hz, 1H), 7.74-7.66 (m, 1H), 7.52 (d, J=7.8 Hz, 1H), 7.49-7.41 (m, 1H), 4.72-3.59 (m, 12H), 2.90-2.70 (m, 1H), 1.50-1.40 (m, 1H), 1.25-1.02 (m, 7H), 0.85-0.74 (m, 1H).

Step 3: (2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octan-6-yl)(thiazol-5-yl)methanone: To a solution of (8-(((6-bromopyridin-2-yl)methoxy)methyl)-2-((S)-2,2-dimethylcyclopropane-1-carbonyl)-2,6-diazaspiro[3.4]octan-6-yl)(thiazol-5-yl)methanone (200 mg, 0.93 mmol) in a mixture of DMA and dioxane (3 mL/12 mL) was added trifluoro(tetrahydro-2H-pyran-4-yl)-14-borane potassium salt (78 mg, 0.40 mmol), Nickel(II) chloride ethylene glycol dimethyl ether complex (10 mg, 0.046 mmol), [Ir{dFCF3ppy}2(bpy)]PF6 (12 mg, 0.011 mmol), dbbpy (12 mg, 0.046 mmol) and 2,6-lutidine (66 mg, 0.6 mmol). The reaction was irradiated with a blue LED for 2.5 hours then was diluted with water and extracted with EtOAc. The combine organic layers were washed with brine, dried over Na₂SO₄, filtered and concentrated. The residue obtined was purified by column chromatography on silica gel (eluent: DCM/MeOH=20/1) to afford (2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octan-6-yl)(thiazol-5-yl)methanone (40 mg, 25%) a yellow semi-solid. LCMS m/z=525.4 [M+H]⁺; ¹HNMR (400 MHz, CD3OD) δ 9.15 (s, 1H), 8.35 (d, J=2.8 Hz, 1H), 7.73 (td, J=7.7, 2.2 Hz, 1H), 7.29 (dd, J=12.3, 6.4 Hz, 1H), 7.21 (d, J=7.8 Hz, 1H), 4.68-3.60 (m, 14H), 3.56 (td, J=11.6, 2.8 Hz, 2H), 3.00-2.88 (m, 1H), 2.85-2.66 (m, 1H), 1.93-1.75 (m, 4H), 1.48-1.34 (m, 1H), 1.21-1.08 (m, 5H), 1.07-1.01 (m, 2H), 0.81-0.71 (m, 1H)

Table 10A: The compounds listed in Table 10A were synthesized from (8-(((6-bromopyridin-2-yl)methoxy)methyl)-2-((S)-2,2-dimethylcyclopropane-1-carbonyl)-2,6-diazaspiro[3.4]octan-6-yl)(thiazol-5-yl)methanone according to the procedures outlined for I-49 using the appropriate commercially available reagents and/or intermediates described elsewhere.

TABLE 10A

| Cmpd. | | | |
|---|---|---|---|
| No. | Compound | ¹HNMR | LCMS |
| I-48 | 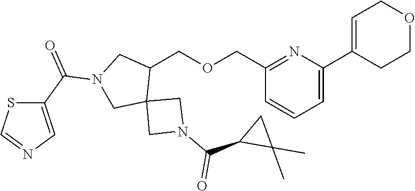 [SMILES: CC1(C)C[C@@H]1C(=O)N1CC2(CN(C(=O)c3cncs3)CC2COCc2cccc(C3=CCOCC3)n2)C1] | ¹H NMR (400 MHz, CD₃OD)δ 9.14 (s, 1H), 8.35 (d, J = 4.9 Hz, 1H), 7.72 (td, J = 7.8, 1.6 Hz, 1H), 7.39 (d, J = 7.9 Hz, 1H), 7.35-7.25 (m, 1H), 6.66 (s, 1H), 4.65-3.58 (m, 16H), 2.84-2.65 (m, 1H), 2.59 (s, 2H), 1.46- 1.33 (m, 1H), 1.23-1.08 (m, 5H), 1.07-1.01 (m, 2H), 0.80-0.70 (m, 1H). | m/z = 523.4 [M + H]⁺ |

### Synthesis of ((S)-2,2-dimethylcyclopropyl)(8-(((6-(tetrahydro-2H-pyran-4-yl)pyridin-2-yl)methoxy)methyl)-6-(thiazolo[4,5-d]pyrimidin-7-yl)-2,6-diazaspiro[3.4]octan-2-yl)methanone (I-47)

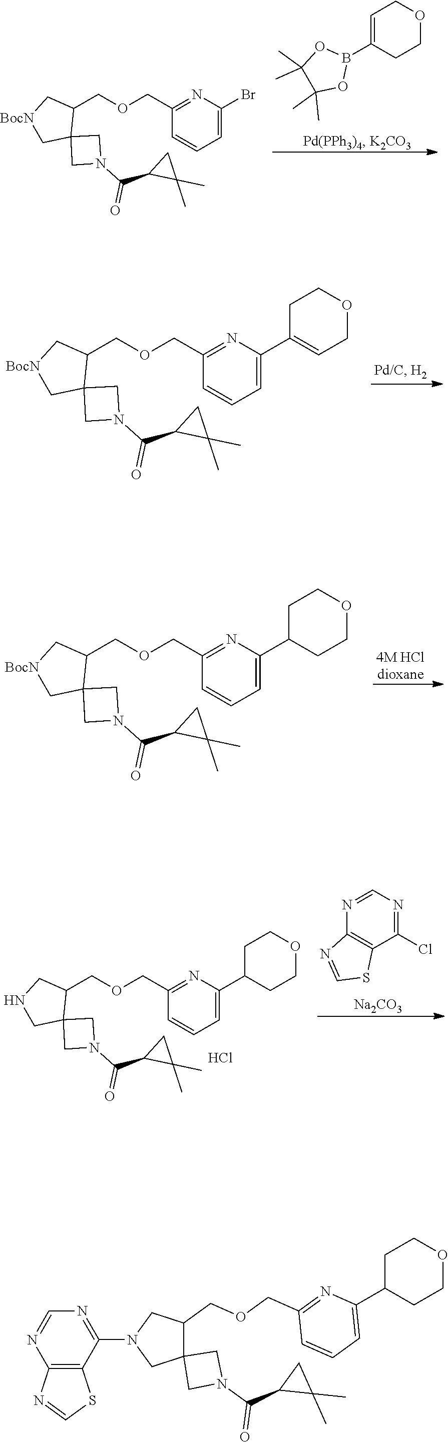

Step 1: tert-butyl 8-(((6-(3,6-dihydro-2H-pyran-4-yl)pyridin-2-yl)methoxy)methyl)-2-((S)-2,2-dimethylcyclopropane-1-carbonyl)-2,6-diazaspiro[3.4]octane-6-carboxylate: To a solution of tert-butyl 8-(((6-bromopyridin-2-yl)methoxy)methyl)-2-((S)-2,2-dimethylcyclopropane-1-carbonyl)-2,6-diazaspiro[3.4]octane-6-carboxylate (400 mg, 0.79 mmol) in a mixture of DME and water (6 mL/1 mL) was added Pd(PPh₃)₄ (185 mg, 0.16 mmol), 2-(3,6-dihydro-2H-pyran-4-yl)-4,4,5,5-tetramethyl-1,3,2-dioxaborolane (200 mg, 0.95 mmol) and K₂CO₃ (218 mg, 1.58 mmol). The reaction was heated at 90° C. under a N₂ atmosphere for 4 hours then was diluted with water (10 mL) and extracted with EtOAc (30 mL×3). The combined organic layers were washed with brine, dried over Na₂SO₄, filtered and concentrated. The residue was purified by column chromatography on silica gel (eluent: DCM:MeOH=50:1) to afford tert-butyl 8-(((6-(3,6-dihydro-2H-pyran-4-yl)pyridin-2-yl)methoxy)methyl)-2-((S)-2,2-dimethylcyclopropane-1-carbonyl)-2,6-diazaspiro[3.4]octane-6-carboxylate (0.27 g, 67%) as a yellow solid. LCMS m/z=512.4 [M+H]⁺

Step 2: tert-butyl 2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octane-6-carboxylate: To a solution of tert-butyl 8-(((6-(3,6-dihydro-2H-pyran-4-yl)pyridin-2-yl)methoxy)methyl)-2-((S)-2,2-dimethylcyclopropane-1-carbonyl)-2,6-diazaspiro[3.4]octane-6-carboxylate (50 mg, 0.10 mmol) in MeOH (10 mL) was added 10% Pd/C (20 mg). The reaction was stirred under H₂ atmosphere at room temperature for 5 hours. The catalyst was removed by filtration through Celite and the filtrate concentrated to afford tert-butyl 2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octane-6-carboxylate (65 mg, 64%) as a colorless oil. LCMS m/z=514.4 [M+H]⁺.

Step 3: ((S)-2,2-dimethylcyclopropyl)(8-(((6-(tetrahydro-2H-pyran-4-yl)pyridin-2-yl)methoxy)methyl)-2,6-diazaspiro[3.4]octan-2-yl)methanone hydrochloride: To a solution of tert-butyl 2-((S)-2,2-dimethylcyclopropane-1-carbonyl)-8-(((6-(tetrahydro-2H-pyran-4-yl)pyridin-2-yl)methoxy)methyl)-2,6-diazaspiro[3.4]octane-6-carboxylate (20 mg, 0.04 mmol) in MeOH (5 mL) was added a solution of HCl in dioxane (4 M, 1 mL). The reaction was stirred at room temperature for 3 hours then the solvent was removed under reduced pressure to afford ((S)-2,2-dimethylcyclopropyl)(8-(((6-(tetrahydro-2H-pyran-4-yl)pyridin-2-yl)methoxy)methyl)-2,6-diazaspiro[3.4]octan-2-yl)methanoneto hydrochloride (18 mg, quant.) as a white solid which was used in the next step directly. LCMS m/z=414.0 [M+H]⁺.

Step 4: ((S)-2,2-dimethylcyclopropyl)(8-(((6-(tetrahydro-2H-pyran-4-yl)pyridin-2-yl)methoxy)methyl)-6-(thiazolo[4,5-d]pyrimidin-7-yl)-2,6-diazaspiro[3.4]octan-2-yl)methanone To a solution of ((S)-2,2-dimethylcyclopropyl)(8-(((6-(tetrahydro-2H-pyran-4-yl)pyridin-2-yl)methoxy)methyl)-2,6-diazaspiro[3.4]octan-2-yl)methanone (16 mg, 0.04 mmol) in a mixture of acetonitrile and DMF (2 mL/1 mL) was added 7-chlorothiazolo[4,5-d]pyrimidine (7 mg, 0.04 mmol) and Na₂CO₃ (8 mg, 0.08 mmol). The reaction was stirred for 2 hours then was diluted with water (10 mL) and extracted with EtOAc (20 mL×2). The combined organic layers were dried over Na₂SO₄, filtered and concentrated. The residue was purified by prep-TLC (DCM:MeOH=15:1) to afford ((S)-2,2-dimethylcyclopropyl)(8-(((6-(tetrahydro-2H-pyran-4-yl)pyridin-2-yl)methoxy)methyl)-6-(thiazolo[4,5-d]pyrimidin-7-yl)-2,6-diazaspiro[3.4]octan-2-yl)methanone (9 mg, 41%) as a yellow oil. LCMS m/z=549.3 [M+H]⁺; ¹H NMR (400 MHz, CD₃OD) δ 9.52 (s, 1H), 8.47 (s, 1H), 7.72-7.60 (m, 1H), 7.32-7.13 (m, 2H), 4.68-3.47 (m, 16H), 3.00-2.75 (m, 2H), 1.93-1.73 (m, 4H), 1.50-1.38 (m, 1H), 1.20-1.02 (m, 7H), 0.80-0.72 (m, 1H).

Table 10B: The compounds listed in Table 10B were synthesized from ((S)-2,2-dimethylcyclopropyl)(8-(((6-(tetrahydro-2H-pyran-4-yl)pyridin-2-yl)methoxy)methyl)-2,6-diazaspiro[3.4]octan-2-yl)methanone hydrochloride according to the procedures outlined for I-47 using the appropriate commercially available reagents and/or intermediates described elsewhere.

TABLE 10B

| Example | | | |
|---|---|---|---|
| Number | Compound | ¹HNMR | LCMS |
| I-46 | 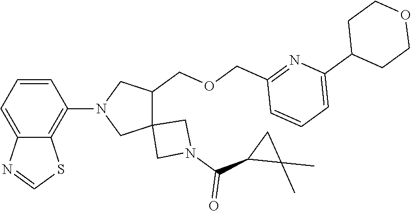 [SMILES: CC1(C)C[C@@H]1C(=O)N1CC2(C1)CN(c1cccc3ncsc13)CC2COCc1cccc(C2CCOCC2)n1] | ¹H NMR (400 MHz, CD₃OD) δ 9.13 (s, 1H), 7.71-7.65 (m, 1H), 7.47 (d, J = 8.0 Hz, 1H), 7.38 (t, J = 8.0 Hz, 1H), 7.31-7.25 (m, 1H), 7.20- 7.14 (m, 1H), 6.66 (d, J = 7.8 Hz, 1H), 4.66-4.27 (m, 4H), 4.23-3.46 (m, 11H), 2.99-2.71 (m, 2H), 1.91-1.74 (m, 4H), 1.46-1.43 (m, 2H), 1.35- 1.31 (m, 2H), 1.18- 1.12 (m, 4H), 1.07-1.02 | m/z = 547.3 [M + H]⁺ |
| | | (m, 2H), 0.80-0.71 (m, | |
| | | 1H). | |

### Synthesis of 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid (1-4)

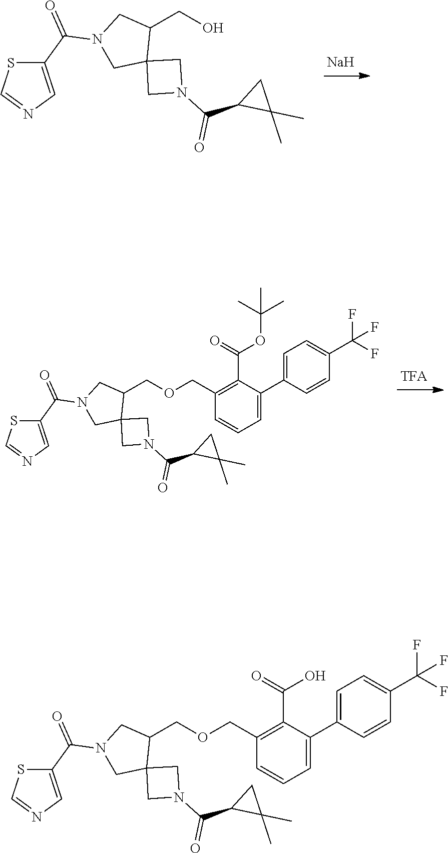

Step 1: tert-butyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate: To a solution of (2-((S)-2,2-dimethylcyclopropane-1-carbonyl)-8-(hydroxymethyl)-2,6-diazaspiro[3.4]octan-6-yl)(thiazol-5-yl)methanone (65 mg, 0.15 mmol) in THF (2 mL) at 0° C. was added NaH (4 mg, 0.17 mmol). After stirring for 0.5 h, tert-butyl 3-(bromomethyl)-4′-(trifluoromethyl)-[1,1′-biphenyl]-2-carboxylate (50 mg, 0.14 mmol, 1.0 eq) was added. The mixture was warmed to room temperature and stirred for 2 h. The mixture was diluted with water (20 mL), extracted with EtOAc (30 mL×2). The combined organic layers were washed with brine, dried over Na₂SO₄, filtered and concentreated under vacuum. The residue was purified by RP-column to afford tert-butyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate (20 mg, 20.1%) as white solid. LCMS m/z=684.5 [M+H]⁺; ¹H NMR (400 MHz, Chloroform-d) δ 8.95 (s, 1H), 8.28 (d, J=15.2 Hz, 1H), 7.70 (d, J=8.0 Hz, 2H), 7.57-7.42 (m, 4H), 7.32-7.30 (m, 1H), 4.70 (d, J=6.0 Hz, 2H), 4.49-3.46 (m, 10H), 2.75-2.60 (m, 1H), 1.40-1.10 (m, 17H), 0.79 (s, 1H).

Step 2: 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid: To a solution of tert-butyl 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylate (60 mg, 0.087 mmol) in DCM (2.0 mL) was added TFA (2 mL) and the reaction stirred at room temperature for 2 h. The solvent was removed and the residue obtained was purified by RP-column to afford 3-(((2-((S)-2,2-dimethylcyclopropane-1-carbonyl)-6-(thiazole-5-carbonyl)-2,6-diazaspiro[3.4]octan-8-yl)methoxy)methyl)-4′-(trifluoromethyl)-[1,1′-biphenyl]-2-carboxylic acid (44 mg, 44%) as white solid. LCMS m/z=628.1 [M+H]⁺; ¹H NMR (400 MHz, Methanol-d₄) δ 9.13 (s, 1H), 8.32 (d, J=12.3 Hz, 1H), 7.70 (d, J=8.0 Hz, 2H), 7.59 (s, 2H), 7.48 (s, 2H), 7.37 (s, 1H), 4.70 (s, 2H), 4.59-3.52 (m, 10H), 2.76-2.58 (m, 1H), 1.47-1.35 (m, 1H), 1.21-1.04 (m, 7H), 0.75 (s, 1H).

### Synthesis of tert-butyl 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (1-57)

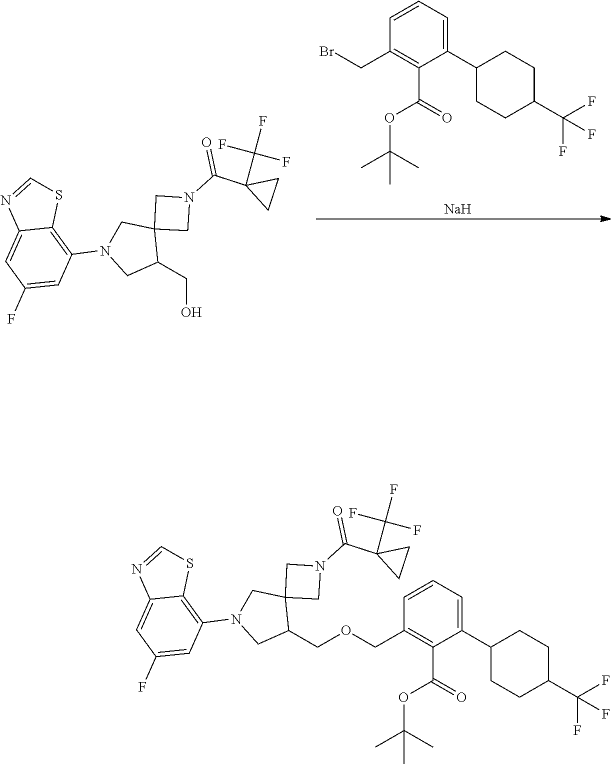

Step 1: tert-butyl 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate: To a solution of (6-(5-fluorobenzo[d]thiazol-7-yl)-8-(hydroxymethyl)-2,6-diazaspiro[3.4]octan-2-yl)(1-(trifluoromethyl)cyclopropyl)methanone (100 mg, 0.2 mmol) and tert-butyl 2-(bromomethyl)-6-(4-(trifluoromethyl)cyclohexyl)benzoate (117 mg, 0.28 mmol) in THE (2 mL) at 0° C. was added NaH (8.4 mg, 0.35 mmol) and the reaction stirred at room temperature overnight. The mixture was diluted with water (20 mL) and extracted with EtOAc (50 mL×2). The combined organic layers were washed with brine, dried over Na₂SO₄, filtered and concentrated. The residue was purified by prep-TLC to afford tert-butyl 2-(((6-(5-fluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (25 mg, 14%) as a white solid. LCMS m/z=770.1 [M+H]⁺; ¹H NMR (400 MHz, Chloroform-d) δ 8.93 (s, 1H), 7.33-7.27 (m, 1H), 7.25-7.16 (m, 3H), 6.27 (dd, J=11.8, 2.2 Hz, 1H), 4.54 (s, 2H), 4.36-3.46 (m, 11H), 2.74-2.59 (m, 2H), 2.43-2.31 (m, 1H), 2.17-2.00 (m, 3H), 1.80-1.66 (m, 5H), 1.56 (s, 9H), 1.19-1.11 (m, 4H).

### Synthesis of 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoicacid (I-56)

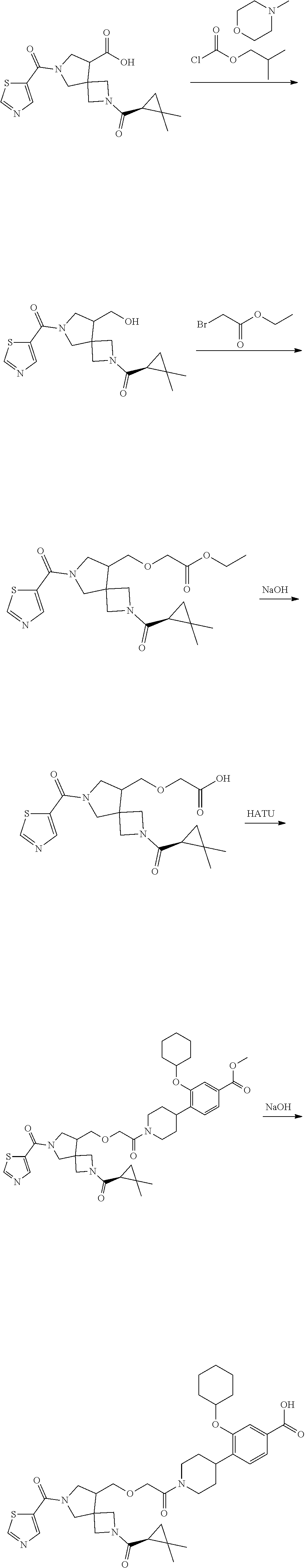

Step 1: (2-((S)-2,2-dimethylcyclopropane-1-carbonyl)-8-(hydroxymethyl)-2,6-diazaspiro[3.4]octan-6-yl)(thiazol-5-yl)methanone: To a solution of 2-((S)-2,2-dimethylcyclopropane-1-carbonyl)-6-(thiazole-5-carbonyl)-2,6-diazaspiro[3.4]octane-8-carboxylic acid (400 mg, 1.1 mmol) in THF (4 mL) at 0° C. was added 4-methylmorpholine (145 mg, 1.4 mmol) and isobutyl chloroformate (210 mg, 1.5 mmol). The reaction was stirred for 30 min then a solution of NaBH₄ (125 mg, 3.3 mmol) in H₂O (1.0 mL) was added dropwise. The reaction was allowed to warm to room temperature and was stirred for 2 h. The mixture was diluted with water (30 mL) and extracted with EtOAc (30 mL×3). The combined organic layers were washed with brine, dried over Na₂SO₄, filtered and concentrated. The residue obtained was purified by column chromatography on silica gel (DCM:MeOH=15:1) to afford (2-((S)-2,2-dimethylcyclopropane-1-carbonyl)-8-(hydroxymethyl)-2,6-diazaspiro[3.4]octan-6-yl)(thiazol-5-yl)methanone (225 mg, 58%) as white solid. LCMS: m/z=349.7 [M+H]⁺; ¹H NMR (400 MHz, CDCl₃) δ 8.91 (s, 1H), 8.25 (d, J=11.8 Hz, 1H), 4.31-3.62 (m, 10H), 2.55 (d, J=22.8 Hz, 1H), 1.15 (s, 8H), 0.80-0.67 (m, 1H).

Step 2: ethyl 2-((2-((S)-2,2-dimethylcyclopropane-1-carbonyl)-6-(thiazole-5-carbonyl)-2,6-diazaspiro[3.4]octan-8-yl)methoxy)acetate: To a solution of (2-((S)-2,2-dimethylcyclopropane-1-carbonyl)-8-(hydroxymethyl)-2,6-diazaspiro[3.4]octan-6-yl)(thiazol-5-yl)methanone (117 mg, 0.3 mmol) in THF (2 mL) at 0° C. was added KI (0.6 mg, 0.003 mmol) and NaH (40 mg, 1.0 mmol). The reaction was stirred for 30 min then ethyl 2-bromoacetate (276 mg, 1.7 mmol) was added. The reaction was allowed to warm to room temperature and was stirred for 4 h. The mixture was diluted with water (30 mL) and extracted with EtOAc (30 mL×3). The combined organic layers were washed with brine, dried over Na₂SO₄, filtered and concentrated. The residue obtained was purified by prep-TLC (DCM:MeOH=10:1) to afford ethyl 2-((2-((S)-2,2-dimethylcyclopropane-1-carbonyl)-6-(thiazole-5-carbonyl)-2,6-diazaspiro[3.4]octan-8-yl)methoxy)acetate (58 mg, 40%) as yellow oil. LCMS: m/z=436.3 [M+H]⁺; ¹H NMR (400 MHz, CDCl₃) δ 8.97 (s, 1H), 8.31 (s, 1H), 4.43 (d, J=25.8 Hz, 1H), 4.22-3.63 (m, 14H), 2.65 (d, J=30.2 Hz, 1H), 1.25 (s, 3H), 1.16 (s, 8H).

Step 3: 2-((2-((S)-2,2-dimethylcyclopropane-1-carbonyl)-6-(thiazole-5-carbonyl)-2,6-diazaspiro[3.4]octan-8-yl)methoxy)acetic acid: To a solution of ethyl 2-((2-((S)-2,2-dimethylcyclopropane-1-carbonyl)-6-(thiazole-5-carbonyl)-2,6-diazaspiro[3.4]octan-8-yl)methoxy)acetate (48 mg, 0.1 mmol) in a mixture of MeOH and water (1 mL/0.5 mL) was added LiOH (9 mg, 0.2 mmol). The reaction was stirred at room temperature for 2 h then was acidified with 1 M HCl to pH˜3 and extracted with 20% MeOH in DCM (20 mL×3). The combined organic layers were washed with brine, dried over Na₂SO₄, filtered and concentrated to afford 2-((2-((S)-2,2-dimethylcyclopropane-1-carbonyl)-6-(thiazole-5-carbonyl)-2,6-diazaspiro[3.4]octan-8-yl)methoxy)acetic acid (45 mg, quant.) as a colorless oil. LCMS: m/z=407.9 [M+H]⁺.

Step 4: methyl 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoate: To a solution of 2-((2-((S)-2,2-dimethylcyclopropane-1-carbonyl)-6-(thiazole-5-carbonyl)-2,6-diazaspiro[3.4]octan-8-yl)methoxy)acetic acid (35 mg, 0.09 mmol) in DCM (1 mL) was added HATU (34 mg, 0.09 mmol) and DIPEA (35 mg, 0.27 mmol). The reaction was stirred for 10 min then methyl 3-(cyclohexyloxy)-4-(piperidin-4-yl)benzoate (34 mg, 0.1 mmol) was added. The reaction was stirred at room temperature for 2 h then the solvent was removed under reduced pressure and the residue obtained purified by prep-TLC (DCM:MeOH=15:1) to afford methyl 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoate (40 mg, 66%) as yellow oil. LCMS: m/z=707.3 [M+H]⁺; ¹H NMR (400 MHz, CDCl₃) δ 9.18 (s, 1H), 8.36 (d, J=26.2 Hz, 1H), 7.54 (d, J=29.6 Hz, 3H), 4.19-3.64 (m, 20H), 2.22 (d, J=7.6 Hz, 1H), 1.51 (d, J=6.8 Hz, 8H), 1.43 (s, 8H), 1.17 (s, 8H).

Step 5: 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoic acid: To a solution of methyl 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoate (30 mg, 0.04 mmol) in MeOH (0.5 mL) was added 10% aqueous NaOH (0.3 mL). The mixture was stirred at room temperature for 2 h then was acidified with 1 M HCl to pH˜3 and extracted with 20% MeOH in DCM (10 mL×3). The combined organic layers were washed with brine, dried over Na₂SO₄, filtered and concentrated. The residue obtained was purified by RP-column to afford 3-(cyclohexyloxy)-4-(1-(2-((2-((S)-2,2-dimethylcyclopropane-1-carbonyl)-6-(thiazole-5-carbonyl)-2,6-diazaspiro[3.4]octan-8-yl)methoxy)acetyl)piperidin-4-yl)benzoic acid (7 mg, 24%) as a white solid. LCMS: m/z=693.2 [M+H]⁺; ¹H NMR (400 MHz, CDCl₃) δ 9.03 (s, 1H), 8.33 (s, 1H), 7.62 (d, J=7.6 Hz, 1H), 7.55 (s, 1H), 7.17 (d, J=8.8 Hz, 1H), 4.34 (d, J=56.2 Hz, 7H), 4.05 (d, J=43.2 Hz, 7H), 3.73 (s, 3H), 3.23 (d, J=12.8 Hz, 1H), 2.71 (d, J=6.6 Hz, 1H), 2.00-1.88 (m, 4H), 1.77 (s, 2H), 1.58 (d, J=13.2 Hz, 5H), 1.43 (d, J=28.8 Hz, 3H), 1.26 (s, 1H), 1.20-1.15 (m, 7H), 0.77 (s, 1H).

### Synthesis of 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (I-52)

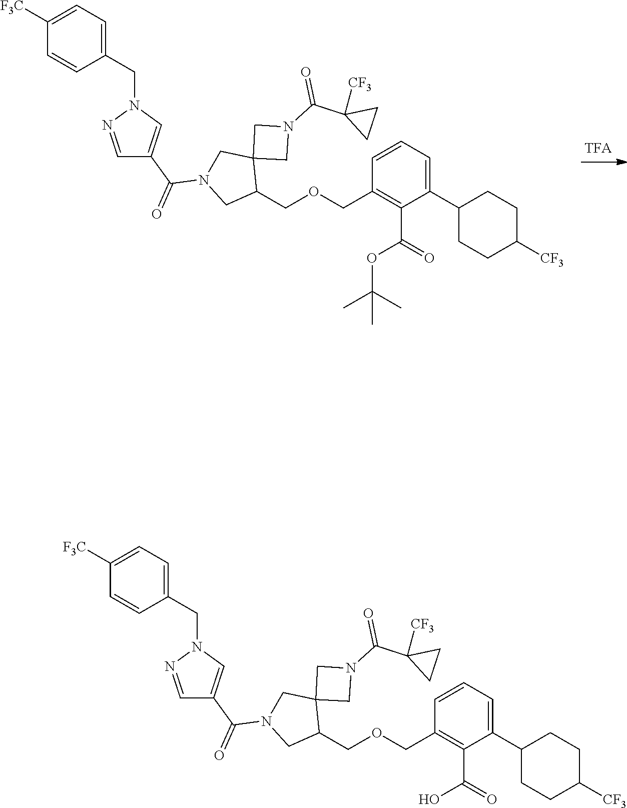 [SMILES: CC(C)(C)OC(=O)c1c(COCC2CN(C(=O)c3cnn(Cc4ccc(C(F)(F)F)cc4)c3)CC23CN(C(=O)C2(C(F)(F)F)CC2)C3)cccc1C1CCC(C(F)(F)F)CC1 ; O=C(O)c1c(COCC2CN(C(=O)c3cnn(Cc4ccc(C(F)(F)F)cc4)c3)CC23CN(C(=O)C2(C(F)(F)F)CC2)C3)cccc1C1CCC(C(F)(F)F)CC1]

Step 1: 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid: To a solution of tert-butyl 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (500 mg, 57.4 mmol) in DCM (5 mL) was added TFA (3 mL) and the reaction stirred at room temperature for 2 h. The solvent was removed under reduced pressure and the residue obtained purified by prep-HPLC to afford 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (330 mg, 70%) as a white solid. LCMS m/z=815.1 [M+H]⁺; ¹H NMR (400 MHz, CD₃OD) δ 8.34 (d, J=6.4 Hz, 1H), 7.94 (d, J=8.5 Hz, 1H), 7.65 (dd, J=8.4, 2.8 Hz, 2H), 7.43 (d, J=7.9 Hz, 2H), 7.35-7.16 (m, 3H), 5.48 (s, 2H), 4.59 (s, 3H), 4.43-4.19 (m, 2H), 4.11-3.89 (m, 3H), 3.80-3.58 (m, 4H), 2.87-2.58 (m, 2H), 2.43 (s, 1H), 2.04 (dt, J=27.9, 13.7 Hz, 3H), 1.72 (q, J=15.8, 11.4 Hz, 4H), 1.16 (d, J=13.3 Hz, 4H).

### Building Blocks

### Synthesis of Methyl 5-(bromomethyl)-3-(methoxymethoxy)-4′-(trifluoromethyl)-[1,1′-biphenyl]-2-carboxylate

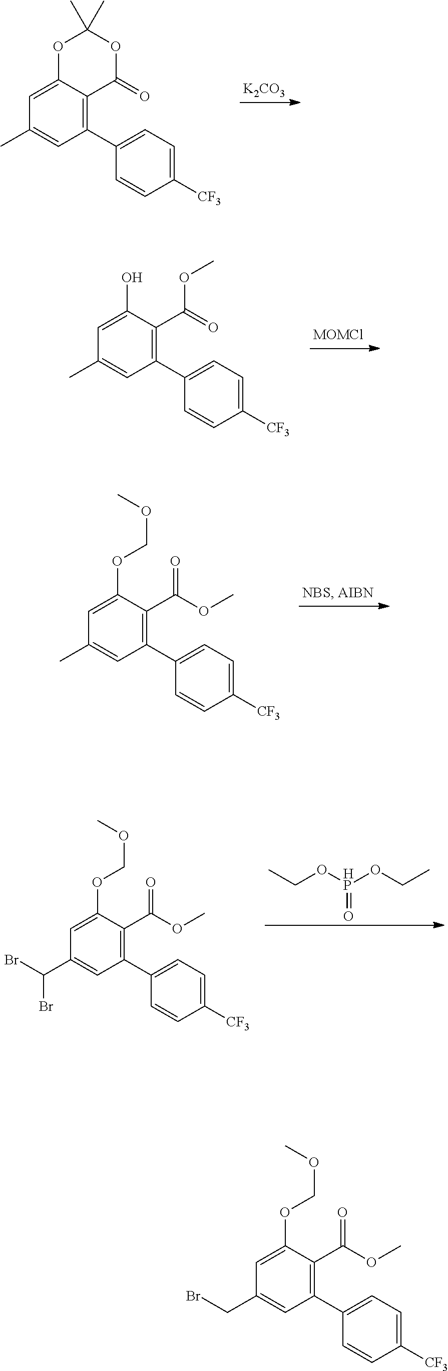

Step 1: methyl 3-hydroxy-5-methyl-4′-(trifluoromethyl)-[1,1′-biphenyl]-2-carboxylate: To a solution of 2,2,7-trimethyl-5-(4-(trifluoromethyl)phenyl)-4H-benzo[d][1,3]dioxin-4-one (524 mg, 1.56 mmol) in MeOH (8 mL) was added K₂CO₃ (237 mg, 1.71 mmol) and the reaction mixture stirred at 50° C. for 4 h. The methanol was removed under reduced pressure and the aquueaous solution acidified to pH˜4 with 1 M HCl and extracted with EtOAc (50 mL×3). The combined organic layers were washed with brine, dried over Na₂SO₄, filtered and concentrated to afford methyl 3-hydroxy-5-methyl-4′-(trifluoromethyl)-[1,1′-biphenyl]-2-carboxylate (430 mg, 89%) as a yellow solid. ¹H NMR (400 MHz, Chloroform-d) δ 10.90 (s, 1H), 7.61 (d, J=8.0 Hz, 2H), 7.36-7.31 (m, 2H), 6.87 (dd, J=1.7, 0.9 Hz, 1H), 6.57 (d, J=1.6 Hz, 1H), 3.47 (s, 3H), 2.35 (s, 3H).

Step 2: methyl 3-(methoxymethoxy)-5-methyl-4′-(trifluoromethyl)-[1,1′-biphenyl]-2-carboxylate: To a solution of methyl 3-hydroxy-5-methyl-4′-(trifluoromethyl)-[1,1′-biphenyl]-2-carboxylate (430 mg, 1.39 mmol) in THF (8 mL) was added NaH (67 mg, 1.66 mmol) and MOMCl (223 mg, 2.77 mmol). The reaction was stirred at room temperature for 2 h, then was diluted with water (30 mL) and extracted with EtOAc (20 mL×3). The combined organic layers were washed with brine, dried over Na₂SO₄, filtered and concentrated to afford methyl 3-(methoxymethoxy)-5-methyl-4′-(trifluoromethyl)-[1,1′-biphenyl]-2-carboxylate (550 mg, 100%) as a yellow oil. ¹H NMR (400 MHz, Chloroform-d) δ 7.64 (d, J=8.0 Hz, 2H), 7.48 (d, J=8.0 Hz, 2H), 7.04 (s, 1H), 6.84 (s, 1H), 5.23 (s, 2H), 3.62 (s, 3H), 3.51 (s, 3H), 2.40 (s, 3H).

Step 3: methyl 5-(dibromomethyl)-3-(methoxymethoxy)-4′-(trifluoromethyl)-[1,1′-biphenyl]-2-carboxylate: To a solution of methyl 3-(methoxymethoxy)-5-methyl-4′-(trifluoromethyl)-[1,1′-biphenyl]-2-carboxylate (550 mg, 1.55 mmol) in CCl₄ (8 mL) was added NBS (346 mg, 1.94 mmol) and AIBN (51 mg, 0.31 mmol). The reaction was heated at 80° C. for 5 h then was diluted with water (100 mL) and extracted with DCM (50 mL×3). The combined organic layers were washed with brine, dried over Na₂SO₄, filtered and concentrated to afford methyl 5-(dibromomethyl)-3-(methoxymethoxy)-4′-(trifluoromethyl)-[1,1′-biphenyl]-2-carboxylate (710 mg, 100%) as a yellow oil.

Step 4: methyl 5-(bromomethyl)-3-(methoxymethoxy)-4′-(trifluoromethyl)-[1,1′-biphenyl]-2-carboxylate: To a solution of methyl 5-(dibromomethyl)-3-(methoxymethoxy)-4′-(trifluoromethyl)-[1,1′-biphenyl]-2-carboxylate (710 mg, 1.39 mmol) in MeCN (8 mL) was added diethyl phosphonate (287 mg, 2.08 mmol) and DIPEA (269 mg, 2.08 mmol). The reaction was stirred at room temperature for 2 h, then was diluted with water (50 mL) and extracted with EtOAc (20 mL×3). The combined organic layers were washed with brine, dried over Na₂SO₄, filtered and concentrated. The residue obtained was purified by prep-TLC (eluent: PE/EtOAc=10/1, 8/1) to afford methyl 5-(bromomethyl)-3-(methoxymethoxy)-4′-(trifluoromethyl)-[1,1′-biphenyl]-2-carboxylate (300 mg, 50%) as a colourless oil. ¹H NMR (400 MHz, Chloroform-d) δ 7.66 (d, J=8.0 Hz, 2H), 7.50 (d, J=8.0 Hz, 2H), 7.25 (d, J=2.0 Hz, 1H), 7.05 (d, J=1.4 Hz, 1H), 5.25 (s, 2H), 4.48 (s, 2H), 3.64 (s, 3H), 3.51 (s, 3H).

### Synthesis of 2-((((S)-6-(4,5-difluorobenzo[d]thiazol-7-yl)-2-((S)-2,2-dimethylcyclopropane-1-carbonyl)-2,6-diazaspiro[3.4]octan-8-yl)methoxy)methyl)-6-(4,4-difluorocyclohexyl)benzoic acid I′-72

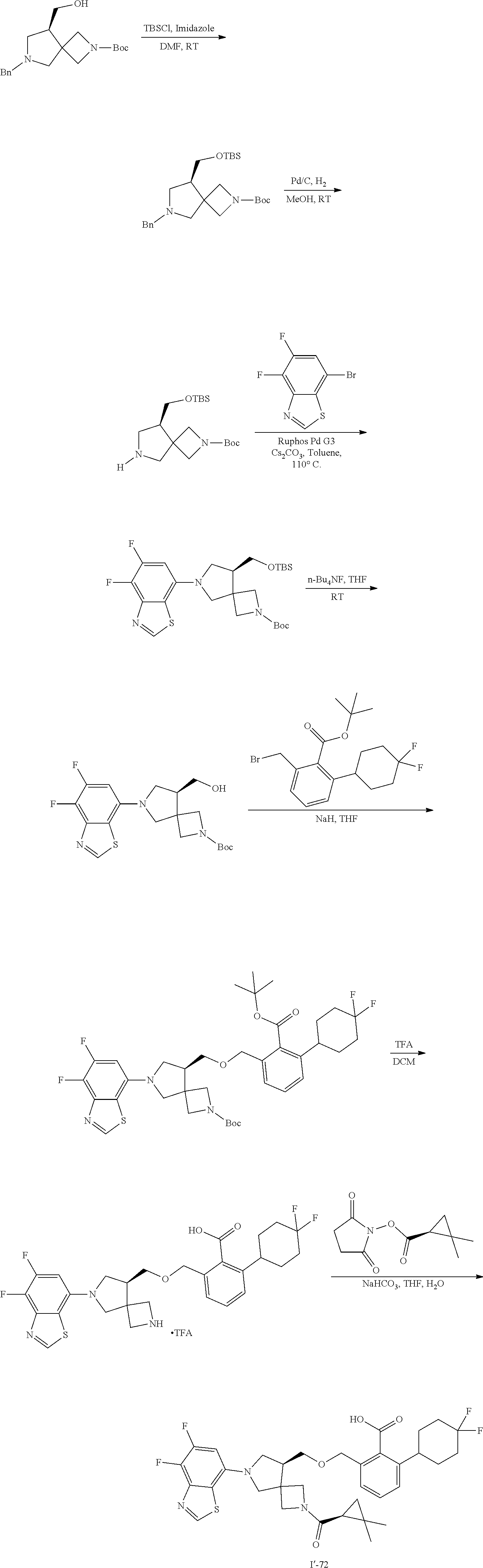

Tert-butyl(S)-6-benzyl-8-(((tert-butyldimethylsilyl)oxy)methyl)-2,6-diazaspiro[3.4]octane-2-carboxylate: A solution of tert-butyl (S)-6-benzyl-8-(hydroxymethyl)-2,6-diazaspiro[3.4]octane-2-carboxylate (3.000 g, 9.03 mmol), imidazole (2.460 g, 36.13 mmol), and TBSCl (2.720 g, 18.06 mmol) in DMF (60 mL) was stirred at room temperature overnight. TLC showed the reaction was completed. The mixture was poured into water (100 mL) and extracted with ethyl acetate (50 mL). The organic layer was collected, and the aqueous layer was extracted with ethyl acetate (50 mL×2). The combined organic layers were washed with brine (100 mL), dried over anhydrous sodium sulfate and concentrated to give a crude residue which was purified by silica gel column chromatography using a 16% ethyl acetate in hexane as a gradient to afford tert-butyl(S)-6-benzyl-8-(((tert-butyldimethylsilyl)oxy)methyl)-2,6-diazaspiro[3.4]octane-2-carboxylate (3.03 g, 74%) as a colorless oil. ¹H NMR (400 MHz, CDCl₃) δ 7.25-7.20 (m, 5H), 4.08 (d, J=9.2 Hz, 2H), 3.79-3.47 (m, 7H), 2.86-2.84 (m, 2H), 2.47 (d, J=8.4 Hz, 2H), 2.20 (d, J=4.8 Hz, 2H), 1.38 (s, 911), 0.84 (s, 9H), 0.00 (s, 6H).

Tert-butyl(S)-8-(((tert-butyldimethylsilyl)oxy)methyl)-2,6-diazaspiro[3.4]octane-2-carboxylate: A mixture of tert-butyl(S)-6-benzyl-8-(((tert-butyldimethylsilyl)oxy)methyl)-2,6-diazaspiro[3.4]octane-2-carboxylate (3.000 g, 6.72 mmol), 20% Pd(OH)₂/C (0.150 g) in MeOH (60 mL) was stirred at room temperature under H₂ overnight. TLC showed the reaction was completed. Palladium on carbon was removed through filtration and washed with methanol (15 mL×2). The combined filtrates were concentrated to afford tert-butyl(S)-8-(((tert-butyldimethylsilyl)oxy)methyl)-2,6-diazaspiro[3.4]octane-2-carboxylate (2.000 g, 83%) as a colorless oil. ¹H NMR (400 MHz, CDCl₃): δ 4.12 (d, J=8.8 Hz, 1H), 3.86-3.60 (m, 5H), 3.13-3.02 (m, 3H), 2.74-2.69 (m, 1H), 2.16-2.14 (m, 1H), 1.43 (s, 9H), 0.88 (s, 9H), 0.05 (s, 6H).

Tert-butyl(S)-6-benzyl-8-(((tert-butyldimethylsilyl)oxy)methyl)-2,6-diazaspiro[3.4]octane-2-carboxylate: A mixture of tert-butyl(S)-8-(((tert-butyldimethylsilyl)oxy)methyl)-2,6-diazaspiro[3.4]octane-2-carboxylate (0.050 g, 0.14 mmol), 7-bromo-4,5-difluorobenzo[d]thiazole (0.035 g, 0.14 mmol), Ruphos Pd G3 (0.023 g, 0.028 mmol), and Cs₂CO₃ (0.069 g, 0.21 mmol) in Toluene (6 mL) was stirred at 110° C. under N₂ for 3 h. TLC showed the reaction was completed. The mixture was poured into water (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate, and concentrated to give a crude residue which was purified by column chromatography using a 12.5% ethyl acetate in petroleum ether gradient to afford tert-butyl(S)-6-benzyl-8-(((tert-butyldimethylsilyl)oxy)methyl)-2,6-diazaspiro[3.4]octane-2-carboxylate (0.024 g, 33%) as a colorless oil. ¹H NMR (400 MHz, CDCl₃) δ 8.94 (s, 1H), 6.37-6.32 (m, 1H), 4.22 (d, J=8.8 Hz, 1H), 3.97 (d, J=8.4 Hz, 1H), 3.87 (d, J=8.4 Hz, 1H), 3.81-3.68 (m, 6H), 3.43-3.39 (m, 1H), 2.50 (m, 1H), 1.45 (s, 9H), 0.87 (s, 9H), 0.05 (d, J=5.6 Hz, 6H).

Tert-butyl(S)-6-benzyl-8-(((tert-butyldimethylsilyl)oxy)methyl)-2,6-diazaspiro[3.4]octane-2-carboxylate: A solution of tert-butyl(S)-6-benzyl-8-(((tert-butyldimethylsilyl)oxy)methyl)-2,6-diazaspiro[3.4]octane-2-carboxylate (0.100 g, 0.19 mmol) and n-Bu₄NF (0.074 g, 0.29 mmol) in dry THE (5 mL) was stirred at room temperature overnight. TLC showed the reaction was completed. The mixture was poured into saturated aq. NH₄Cl solution (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate, and concentrated to give a crude residue which was purified by silica gel column chromatography using a 50% ethyl acetate in petroleum ether as a gradient to afford tert-butyl(S)-6-benzyl-8-(((tert-butyldimethylsilyl)oxy)methyl)-2,6-diazaspiro[3.4]octane-2-carboxylate (0.055 g, 55%) as a colorless oil. ¹H NMR (400 MHz, CDCl₃): δ 8.95 (s, 1H), 6.39-6.34 (m, 1H), 4.20 (d, J=9.2 Hz, 1H), 3.98-3.90 (m, 3H), 3.83-3.71 (m, 5H), 3.50-3.46 (m, 1H), 2.56 (m, 1H), 1.45 (s, 9H).

Tert-butyl(S)-6-benzyl-8-(((tert-butyldimethylsilyl)oxy)methyl)-2,6-diazaspiro[3.4]octane-2-carboxylate: A mixture of tert-butyl(S)-6-benzyl-8-(((tert-butyldimethylsilyl)oxy)methyl)-2,6-diazaspiro[3.4]octane-2-carboxylate (0.150 g, 0.36 mmol), NaH (0.058 g, 1.80 mmol in mineral oil) in dry DMF (6 mL) was stirred at room temperature for 1 h, followed by the addition of tert-butyl 2-(bromomethyl)-6-(4,4-difluorocyclohexyl)benzoate, see synthesis of I-101 (0.213 g, 0.54 mmol), and the resulting mixture was stirred at 40° C. overnight. TLC showed the reaction was completed. The mixture was poured into ice water (20 mL) and extracted with ethyl acetate (10 mL). The organic layer was collected, and the aqueous layer was extracted with ethyl acetate (10 mL×2). The combined organic layers were washed with brine (15 mL), dried over anhydrous sodium sulfate and concentrated to give a crude residue which was purified by silica gel column chromatography using a 25% ethyl acetate in petroleum ether gradient to afford tert-butyl(S)-6-benzyl-8-(((tert-butyldimethylsilyl)oxy)methyl)-2,6-diazaspiro[3.4]octane-2-carboxylate (0.203 g, 79%) as a yellow oil. MS: [MH]⁺ 720.40.

Tert-butyl(S)-6-benzyl-8-(((tert-butyldimethylsilyl)oxy)methyl)-2,6-diazaspiro[3.4]octane-2-carboxylate TFA salt: A solution of tert-butyl(S)-6-benzyl-8-(((tert-butyldimethylsilyl)oxy)methyl)-2,6-diazaspiro[3.4]octane-2-carboxylate (0.023 g, 0.03 mmol) and TFA (1 mL) in DCM (1 mL) was stirred at room temperature overnight. LCMS showed the reaction was completed. The reaction mixture was concentrated to afford a crude product (0.020 g, 100%) as yellow oil which was used in next step without further purification. MS: [MH]-564.25. [00425]2-((((8S)-6-(4,5-difluorobenzo[d]thiazol-7-yl)-2-(2,2-dimethylcyclopropane-1-carbonyl)-2,6-diazaspiro[3.4]octan-8-yl)methoxy)methyl)-6-(4,4-difluorocyclohexyl)benzoic acid I′-72: To a solution of tert-butyl(S)-6-benzyl-8-(((tert-butyldimethylsilyl)oxy)methyl)-2,6-diazaspiro[3.4]octane-2-carboxylate TFA salt (0.020 g, 0.03 mmol) and NaHCO₃ (0.054 g, 0.60 mmol) in H₂O (2 mL) at room temperature was added 2,5-dioxopyrrolidin-1-yl (S)-2,2-dimethylcyclopropane-1-carboxylate (0.016 g, 0.06 mmol) in THF (2 mL), the resulting mixture was stirred at RT overnight. TLC showed the reaction was completed. The reaction mixture was acidified with hydrochloric acid (2 M) to pH 4-5, extracted with ethyl acetate (10 mL×3), dried over sodium sulfate, and concentrated to give a residue which was purified through column chromatography using 3% dichloromethane in methanol gradient to afford 2-((((8S)-6-(4,5-difluorobenzo[d]thiazol-7-yl)-2-(2,2-dimethylcyclopropane-1-carbonyl)-2,6-diazaspiro[3.4]octan-8-yl)methoxy)methyl)-6-(4,4-difluorocyclohexyl)benzoic acid I′-72 (0.012 g, 28%) as a white solid. ¹H NMR (400 MHz, CD₃OD) 9.21 (s, 1H), 7.32-7.24 (m, 3H), 6.55-6.51 (m, 1H), 4.60 (s, 1H), 4.42 (dd, J=9.60 Hz, 33.20 Hz, 1H), 4.27-4.23 (m, 1H), 4.16 (d, J=8.80 Hz, 1H), 3.99 (dd, J=12.00 Hz, 44.00 Hz, 1H), 3.86-3.71 (m, 5H), 3.69-3.62 (m, 1H), 3.52-3.46 (m, 1H), 2.88-2.65 (m, 2H), 2.15-2.10 (m, 2H), 1.87-1.76 (m, 6H), 1.45-1.36 (m, 1H), 1.15-1.12 (m, 5H), 1.05-1.03 (m, 2H), 0.90-0.85 (m, 1H), 0.75-0.70 (m, 1H). MS: [MH]⁺ 660.20.

The following compounds were prepared in a manner analogous to the procedures described above for 2-((((8S)-6-(4,5-difluorobenzo[d]thiazol-7-yl)-2-(2,2-dimethylcyclopropane-1-carbonyl)-2,6-diazaspiro[3.4]octan-8-yl)methoxy)methyl)-6-(4,4-difluorocyclohexyl)benzoic acid I′-72. [00427](S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-71 (0.011 g, yield 9%) as a white solid. ¹H NMR (400 MHz, DMSO-d₆116 9.22 (s, 1H), 7.35-7.26 (m, 3H), 6.54 (dd, J=7.6 Hz, 1H), 4.61-4.58 (m, 2H), 4.38-4.25 (m, 1H), 4.15-3.95 (m, 1H), 3.80-3.67 (m, 5H), 3.50-3.48 (m, 1H), 2.85-2.82 (m, 1H), 2.73-2.70 (m, 1H), 2.15-2.14 (m, 2H), 1.94-1.81 (s, 6H), 1.40-1.26 (m, 8H), 0.92-0.89 (m, 1H). MS: [MH]⁺ 702.3.

(S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4-methylcyclohexyl)benzoic acid I′-47 (0.018 g, 12.3%) as a white solid. ¹HNMR (400 MHz, CD₃OD): δ 9.20 (m, 1H), 7.35-7.29 (m, 2H), 7.20-7.18 (m, 1H), 6.56-6.51 (m, 1H), 4.79-4.25 (m, 4H), 4.04-3.46 (m, 7H), 2.73-2.61 (m, 2H), 1.97-1.49 (m, 9H), 1.38-1.30 (m, 8H), 1.11-0.93 (m, 4H). MS: [MH]⁺ 680.20.

### Synthesis of 2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4,4-difluorocyclohexyl)benzamide I′-33

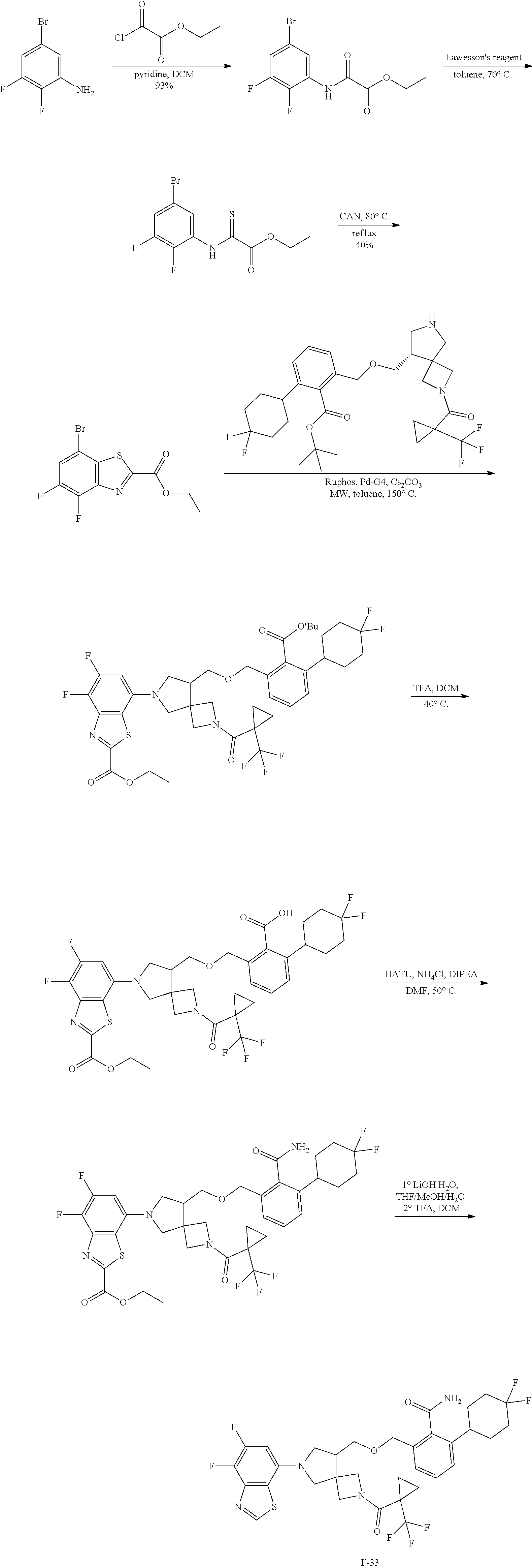

Ethyl 2-((5-bromo-2,3-difluorophenyl)amino)-2-oxoacetate: To a solution of 5-bromo-2,3-difluoroaniline (1.000 g, 4.8 mmol) in dichloromethane (20 mL) at 0° C. was added pyridine (0.456 g, 5.8 mmol) and ethyl 2-chloro-2-oxoacetate (787.7 mg, 5.8 mmol) dropwise. The mixture was allowed to warm to room temperature and stirred at room temperature under nitrogen atmosphere for 2 hours. TLC showed the reaction was completed. The reaction mixture was diluted with dichloromethane (20 mL), washed with diluted hydrochloride acid (1 N, 20 mL×2) and brine (25 mL), dried over anhydrous sodium sulfate, and concentrated under reduced pressure to give a crude residue which was purified by silica gel flash chromatography using 10% ethyl acetate in hexane gradient to afford ethyl 2-((5-bromo-2,3-difluorophenyl)amino)-2-oxoacetate (1.400 g, 95% yield) as yellow oil. ¹HNMR (400 MHz, CDCl₃): δ 9.10 (br, 1H), 8.41-8.34 (m, 1H), 7.22-7.14 (m, 1H), 4.51-4.39 (m, 2H), 1.44 (t, J=7.2 Hz, 3H). MS: [MH]⁺ 309.75.

Ethyl 2-((5-bromo-2,3-difluorophenyl)amino)-2-thioxoacetate: A mixture of ethyl 2-((5-bromo-2,3-difluorophenyl)amino)-2-oxoacetate (1.400 g, 4.60 mmol) and Lawesson's reagent (3.700 g, 9.20 mmol) in toluene (20 mL) was stirred at 70° C. under nitrogen atmosphere overnight. The mixture was cooled to room temperature, diluted with ethyl acetate (20 mL), washed with saturated aqueous sodium bicarbonate solution (15 mL×2) and brine (20 mL), dried over sodium sulfate, and concentrated under reduced pressure to give a crude residue which was purified by silica gel flash chromatography using 2.5% ethyl acetate in hexane gradient to afford ethyl 2-((5-bromo-2,3-difluorophenyl)amino)-2-thioxoacetate (1.200 g, 80% yield) as red solid. ¹HNMR (400 MHz, CDCl₃): δ 10.70 (br, 1H), 9.01-8.94 (m, 1H), 7.33-7.27 (m, 1H), 4.49-4.41 (m, 2H), 1.45 (t, J=6.8 Hz, 1H). MS: [MH]⁺ 323.70.

Ethyl 7-bromo-4,5-difluorobenzo[d]thiazole-2-carboxylate: To a solution of ethyl 2-((5-bromo-2,3-difluorophenyl)amino)-2-thioxoacetate (0.900 g, 2.8 mmol) in acetonitrile (20 mL) was added ammonium ceric nitrate (3.100 g, 5.8 mmol). The mixture was stirred at 80° C. for 10 minutes. TLC showed the reaction was completed. The mixture was cooled to room temperature and partitioned between ethyl acetate (30 mL) and water (15 mL); the organic layer was collected, washed with brine (20 mL), dried over sodium sulfate, and concentrated under reduced pressure to give a crude residue which was purified by silica gel flash chromatography using 2.5% ethyl acetate in hexane gradient to afford ethyl 7-bromo-4,5-difluorobenzo[d]thiazole-2-carboxylate (0.370 g, 41% yield) as off-white solid. MS: [MH]⁺323.75.

Ethyl 7-(8-(((2-(tert-butoxycarbonyl)-3-(4,4-difluorocyclohexyl)benzyl)oxy) methyl)-2-(1-(trifluoromethyl)cyclopropane-1-carbonyl)-2,6-diazaspiro[3.4]octan-6-yl)-4,5-difluorobenzo[d]thiazole-2-carboxylate: A mixture of tert-butyl (S)-2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate, see synthesis of I′-57 (0.120 g, 0.20 mmol), ethyl 7-bromo-4,5-difluorobenzo[d]thiazole-2-carboxylate (0.079 g, 0.24 mmol), RuPhosPdG₄ (0.017 g, 0.020 mmol), and Cesium carbonate (0.098 g, 0.30 mmol) in toluene (4 mL) was stirred at 150° C. under N₂ under microwave irradiation for 1 hour. The reaction was cooled to room temperature and filtered through a pad. The filtrate was concentrated under reduced pressure to give a crude residue which was purified by preparative TLC using 33% ethyl acetate in petroleum ether gradient to afford ethyl 7-(8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluorobenzo[d]thiazole-2-carboxylate (0.053 g, yield 29%) as yellow solid. MS: [MH]⁺ 828.20.

2-(4,4-Difluorocyclohexyl)-6-(((6-(2-(ethoxycarbonyl)-4,5-difluorobenzo[d]thiazol-7-yl)-2-(1-(trifluoromethyl)cyclopropane-1-carbonyl)-2,6-diazaspiro[3.4]octan-8-yl)methoxy)methyl)benzoic acid: To a solution of ethyl 7-(8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluorobenzo[d]thiazole-2-carboxylate (0.050 g, 0.06 mmol) in dichloromethane (1 mL) was added trifluoroacetic acid (1 mL) and the mixture was stirred at 40° C. for 1 hour. TLC showed the reaction was completed. The mixture was concentrated under reduced pressure to give a crude residue which was purified by preparative TLC using 5% methanol in dichloromethane gradient to afford 2-(4,4-difluorocyclohexyl)-6-(((6-(2-(ethoxycarbonyl)-4,5-difluorobenzo[d]thiazol-7-yl)-2-(1-(trifluoromethyl)cyclopropane-1-carbonyl)-2,6-diazaspiro[3.4]octan-8-yl)methoxy)methyl)benzoic acid (0.044 g, yield 94%) as yellow solid. MS: [MH]⁺ 772.20.

Ethyl 7-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluorobenzo[d]thiazole-2-carboxylate: To a mixture of 2-(4,4-difluorocyclohexyl)-6-(((6-(2-(ethoxycarbonyl)-4,5-difluorobenzo[d]thiazol-7-yl)-2-(1-(trifluoromethyl)cyclopropane-1-carbonyl)-2,6-diazaspiro[3.4]octan-8-yl)methoxy)methyl)benzoic acid (0.100 g, 0.13 mmol) in N,N-dimethylformamide (3 mL) was added ammonium chloride (0.028 g, 0.52 mmol), N,N-diisopropylethylamine (0.067 g, 0.52 mmol), and 2-(7-Aza-1H-benzotriazole-1-yl)-1,1,3,3-tetramethyluronium hexafluorophosphate (0.059 g, 0.16 mmol). The mixture was stirred at 50° C. under nitrogen atmosphere overnight. TLC showed the reaction was completed. The mixture was cooled to room temperature and partitioned between ethyl acetate (15 mL) and water (8 mL×2); the organic layer was washed with brine (10 mL), dried over sodium sulfate, and concentrated under reduced pressure to give a crude residue which was purified by silica gel flash chromatography using 5% methanol in dichloromethane gradient to afford ethyl 7-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluorobenzo[d]thiazole-2-carboxylate (0.050 g, 50% yield) as a yellow solid. [MH]⁺ 771.20.

2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4,4-difluorocyclohexyl)benzamide I′-33: A mixture of ethyl 7-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluorobenzo[d]thiazole-2-carboxylate (0.050 g, 0.065 mmol) and lithium hydroxide monohydrate (0.013 g, 0.32 mmol) in tetrahydrofuran (2 mL)—water (0.5 mL)—methanol (0.5 mL) was stirred at room temperature for 1 hour. TLC showed the reaction was completed. The mixture was acidified with diluted hydrochloride acid (1 N) till pH 6, and extracted with dichloromethane (5 mL×3). The combined organic layers were washed with brine (10 mL), dried over sodium sulfate, and concentrated under reduced pressure to give a crude residue which was dissolved in dichloromethane (1 mL) containing 2,2,2-trifluoroacetic acid (1 drop). The mixture was stirred at room temperature for 2 hours. The reaction mixture was concentrated under reduced pressure to give a crude residue which was purified by prep-HPLC to afford product 2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4,4-difluorocyclohexyl)benzamide (0.006 g, 13% yield) as grey solid. ¹HNMR (400 MHz, CD₃OD): δ 9.22 (s, 1H), 7.35-7.25 (m, 3H), 6.60-6.51 (m, 1H), 4.77-3.87 (m, 6H), 3.84-3.68 (m, 5H), 3.52-3.45 (m, 1H), 2.90-2.80 (m, 1H), 2.79-2.71 (m, 1H), 2.19-2.08 (m, 2H), 1.99-1.77 (m, 6H), 1.23-1.06 (m, 4H). MS: [MH]⁺ 699.55.

The following compounds were prepared in a manner analogous to the procedures described above for 2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4,4-difluorocyclohexyl)benzamide I′-33

7-(8-(((3-(4,4-difluorocyclohexyl)benzyl)oxy)methyl)-2-(1-(trifluoromethyl)cyclopropane-1-carbonyl)-2,6-diazaspiro[3.4]octan-6-yl)-4,5-difluoro-N-methylbenzo[d]thiazole-2-carboxamide I′-9 (0.008 g, yield 39%) as a white solid. ¹HNMR (400 MHz, CD₃OD): δ 7.24-7.12 (m, 4H), 6.56-6.52 (m, 1H), 4.72-3.84 (m, 6H), 3.81-3.69 (m, 5H), 3.51-3.47 (m, 1H), 2.97 (s, 3H), 2.77-2.70 (m, 1H), 2.62-2.56 (m, 1H), 2.13-2.05 (m, 2H), 1.96-1.65 (m, 6H), 1.19-1.17 (m, 4H). LCMS: [MH]⁺ 713.15.

### Synthesis of (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetic acid I-102

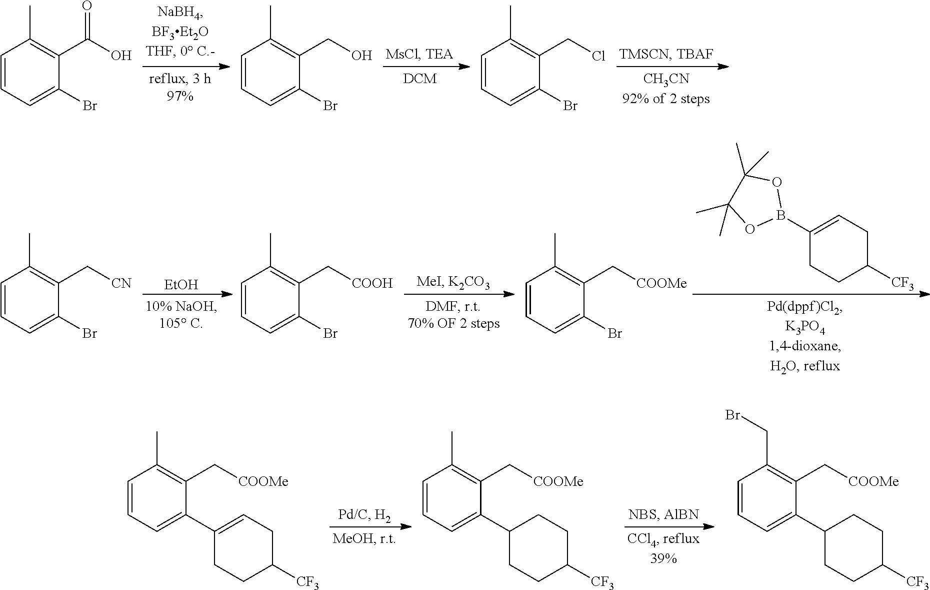

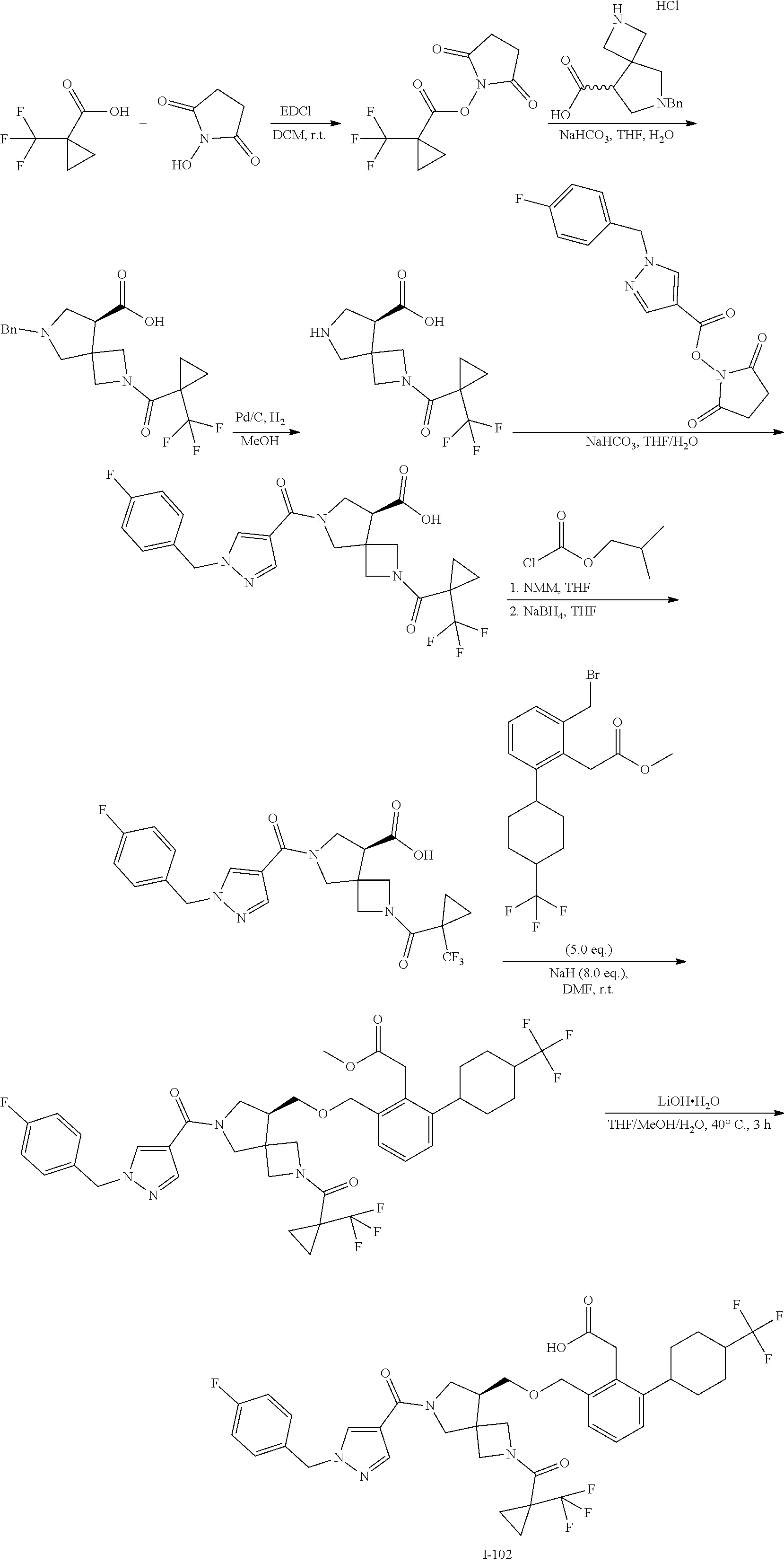

(2-Bromo-6-methylphenyl)methanol: To a stirred mixture of 2-bromo-6-methylbenzoic acid (30.000 g, 139.53 mmol) in tetrahydrofuran at 0° C. under nitrogen atomsphere was added slowly sodium borohydride (22.000 g, 578.94 mmol) and boron trifluoride diethyl etherate (160.000 g, 1126.76 mmol), and the resulting mixture was stirred at 65° C. for 3 h. TLC showed the reaction was complete. The reaction mixture was poured into ice water (100 mL), and extracted with ethyl acetate (500 mL). The organic layer was collected, and the aqueous layer was extracted with ethyl acetate (150 mL×2). The combined organic layers were washed with brine (200 mL), dried over anhydrous sodium sulfate, and concentrated to give a crude residue which was purified through silica gel flash column chromatography using 5% ethyl acetate in hexane gradient to afford (2-bromo-6-methylphenyl)methanol (29.000 g, yield 97%) as a white solid. ¹HNMR (400 MHz, CDCl₃): δ 7.41 (d, J=8.0 Hz, 1H), 7.14 (d, J=7.6 Hz, 1H), 7.05 (t, J=7.8 Hz, 1H), 4.85 (s, 2H), 2.48 (s, 3H).

1-Bromo-2-(chloromethyl)-3-methylbenzene: To a stirred mixture of (2-bromo-6-methylphenyl)methanol (2.000 g, 10.00 mmol) and triethylamine (2.020 g, 20.00 mmol) in dichloromethane (50 mL) at 0° C. was added methanesulfonyl chloride (1.370 g, 12.00 mmol) dropwise, the resulting mixture was stirred under nitrogen atomsphere for 3 hours. TLC showed the reaction was complete. The mixture was concentrated to give a crude residue which was purified through silica gel flash column chromatography using 100% dichloromethane gradient to afford 1-bromo-2-(chloromethyl)-3-methylbenzene (1.900 g, yield 87%) as a colorless oil. ¹HNMR (400 MHz, CDCl₃): δ 7.44 (d, J=7.6 Hz, 1H), 7.14 (d, J=7.6 Hz, 1H), 7.07 (t, J=7.8 Hz, 1H), 4.81 (s, 2H), 2.49 (s, 3H).

2-(2-Bromo-6-methylphenyl)acetonitrile: A mixture of 1-bromo-2-(chloromethyl)-3-methylbenzene (1.900 g, 6.80 mmol), trimethylsilyl cyanide (1.030 g, 10.20 mmol), and tetrabutylammonium fluoride (2.670 g, 10.20 mmol) in acetonitrile (35 mL) was refluxed under nitrogen atomsphere overnight. TLC showed the reaction was complete. The reaction mixture was concentrated to give a crude residue which was purified through silica gel flash column chromatography using a 10% ethyl acetate in hexane gradient to afford 2-(2-bromo-6-methylphenyl)acetonitrile (0.180 g, yield 98%) as a white solid. ¹HNMR (400 MHz, CDCl₃): δ 7.47 (d, J=8.0 Hz, 1H), 7.18 (d, J=7.6 Hz, 1H), 7.10 (t, J=7.8 Hz, 1H), 3.89 (s, 2H), 2.47 (s, 3H).

2-(2-Bromo-6-methylphenyl)acetic acid: A mixture of 2-(2-bromo-6-methylphenyl)acetonitrile (1.000 g, 4.76 mmol) in sodium hydroxide solution (15 mL, 10% in water) was refluxed under nitrogen atomsphere overnight. TLC showed the reaction was complete. The reaction mixture was acidified with hydrochloric acid (2N) to pH 5, and extracted with dichloromethane (10 mL×3). The combined organic layers were dried over anhydrous sodium sulfate and concentrated to afford 2-(2-bromo-6-methylphenyl)acetic acid (1.000 g, yield 91%) as a white solid. ¹HNMR (400 MHz, DMSO-d6): δ 12.46 (s, 1H), 7.44 (d, J=8.0 Hz, 1H), 7.20 (d, J=7.6 Hz, 1H), 7.10 (t, J=7.8 Hz, 1H), 3.78 (s, 2H), 2.30 (s, 3H).

Methyl 2-(2-bromo-6-methylphenyl)acetate: A mixture of 2-(2-bromo-6-methylphenyl)acetic acid (1.000 g, 4.37 mmol), iodomethane (0.930 g, 6.60 mmol), and potassium carbonate (0.911 g, 6.60 mmol) in N,N-dimethylformamide (20 mL) was stirred at room temperature under nitrogen atomsphere for 1 hour. TLC showed the reaction was complete. The reaction mixture was poured into water (50 mL) and extracted with ethyl acetate (30 mL) and. The organic layer was collected, and the aqueous layer was extracted with ethyl acetate (20 mL×2). The combined organic layers were washed with brine (30 mL), dried over anhydrous sodium sulfate, and concentrated to give a crude residue which was purified through silica gel flash column chromatography using a 5% ethyl acetate in hexane a gradient to afford methyl 2-(2-bromo-6-methylphenyl)acetate (0.800 g, yield 75%) as a yellow oil. ¹HNMR (400 MHz, CDCl₃): δ 7.43 (d, J=8.0 Hz, 1H), 7.13 (d, J=7.6 Hz, 1H), 7.03 (t, J=7.8 Hz, 1H), 3.90 (s, 2H), 3.70 (s, 3H), 2.34 (s, 3H).

Methyl 2-(3-methyl-4′-(trifluoromethyl)-2′,3′,4′,5′-tetrahydro-[1,1′-biphenyl]-2-yl)acetate: A mixture of methyl 2-(2-bromo-6-methylphenyl)acetate (0.900 g, 3.72 mmol), 4,4,5,5-tetramethyl-2-(4-(trifluoromethyl)cyclohex-1-en-1-yl)-1,3,2-dioxaborolane (2.050 g, 7.44 mmol), [1,1′-bis(diphenylphosphino)ferrocene]dichloropalladium(II) (0.269 g, 0.37 mmol), and potassium phosphate (1.570 g, 7.44 mmol) in 1,4-dioxane (16 mL) and water (4 mL) was stirred at 100° C. under nitrogen atomsphere overnight. TLC showed the reaction was complete. The reaction mixture was poured into water (30 mL) and extracted with ethyl acetate (30 mL) and. The organic layer was collected, and the aqueous layer was extracted with ethyl acetate (20 mL×2). The combined organic layers were washed with brine (30 mL), dried over anhydrous sodium sulfate, and concentrated to give a crude residue which was purified through silica gel flash column chromatography using a 2% ethyl acetate in hexane gradient to afford methyl 2-(3-methyl-4′-(trifluoromethyl)-2′,3′,4′,5′-tetrahydro-[1,1′-biphenyl]-2-yl)acetate (1.600 g, crude) as a colorless solid. ¹HNMR (400 MHz, CDCl₃): δ 7.26-7.09 (m, 2H), 6.95 (d, J=6.8 Hz, 1H), 5.54 (s, 1H), 3.69 (s, 3H), 3.68 (s, 2H), 2.41-2.32 (m, 3H), 2.29 (s, 3H), 2.13-2.09 (m, 3H), 1.72-1.68 (m, 1H).

Methyl 2-(2-methyl-6-(4-(trifluoromethyl)cyclohexyl)phenyl)acetate: A mixture of methyl 2-(3-methyl-4′-(trifluoromethyl)-2′,3′,4′,5′-tetrahydro-[1,1′-biphenyl]-2-yl)acetate (1.6 g, crude) and Pd/C (0.320 g) in methanol (30 mL) was stirred at room temperature under hydrogen atomsphere for 2 hours. TLC showed the reaction was complete. The mixture was filtered and the filtrate was concentrated under reduced pressure to afford methyl 2-(2-methyl-6-(4-(trifluoromethyl)cyclohexyl)phenyl)acetate (1.230 g, yield 75%) as a colorless oil. ¹HNMR (400 MHz, CDCl₃): δ 7.20-7.13 (m, 2H), 7.05 (d, J=7.2 Hz, 1H), 3.76 (s, 2H), 3.60 (s, 3H), 2.84-2.79 (m, 1H), 2.41-2.36 (m, 1H), 2.35 (s, 3H), 2.16-2.13 (m, 2H), 1.75-1.63 (m, 6H).

Methyl 2-(2-(bromomethyl)-6-(4-(trifluoromethyl)cyclohexyl)phenyl)acetate: A mixture of methyl 2-(2-methyl-6-(4-(trifluoromethyl)cyclohexyl)phenyl)acetate (0.050 g, 0.16 mmol), n-bromosuccinimide (0.028 g, 0.16 mmol), and 2,2′-azobis(2-methylpropionitrile) (0.003 g, 0.01 mmol) in CCl₄ (5 mL) was reflux under nitrogen atomsphere overnight. TLC showed the reaction was complete. The mixture was poured into water (5 mL) and extracted with DCM (5 mL×2). The combined organic layers see synthesis of 5 prime −43 were washed with brine (5 mL), dried over anhydrous sodium sulfate, and concentrated to give a crude residue which was purified through silica gel flash column chromatography using a 2% ethyl acetate in hexane gradient to afford methyl 2-(2-(bromomethyl)-6-(4-(trifluoromethyl)cyclohexyl)phenyl)acetate (0.025 g, yield 40%) as a colorless oil. ¹HNMR (400 MHz, CDCl₃): δ 7.10-7.20 (m, 3H), 4.60 (s, 2H), 3.90 (s, 2H), 3.70 (s, 3H), 2.80-2.85 (m, 1H), 2.38-2.41 (m, 1H), 2.14-2.17 (m, 2H), 1.62-1.73 (m, 6H).

2,5-Dioxopyrrolidin-1-yl 1-(trifluoromethyl)cyclopropane-1-carboxylate: A mixture of 1-(trifluoromethyl)cyclopropane-1-carboxylic acid (20.000 g, 129.87 mmol), 1-hydroxypyrrolidine-2,5-dione (14.930 g, 129.87 mmol) and CDI (30.000 g, 156.25 mmol) in dichloromethane was stirred at room temperature under nitrogen atomsphere overnight. The reaction mixture was concentrated to give a crude residue which was purified by silica gel flash column chromatography using a dichloromethane gradient to afford 2,5-dioxopyrrolidin-1-yl 1-(trifluoromethyl)cyclopropane-1-carboxylate (29.500 g, yield 90%) as a white solid. ¹HNMR (400 MHz, CDCl₃): δ 2.83 (s, 4H), 1.78-1.75 (m, 2H), 1.61-1.58 (m, 2H).

(S)-6-benzyl-2-(1-(trifluoromethyl)cyclopropanecarbonyl)-2,6-diazaspiro[3.4]octane-8-carboxylic acid: To a solution of (S)-6-benzyl-2,6-diazaspiro[3.4]octane-8-carboxylic acid hydrochloride (5.200 g, 18.44 mmol) in water (40 mL) at 0° C. was added sodium bicarbonate (6.100 g, 72.17 mmol), the resulting mixture was stirred at room temperature for 20 min, followed by the addition of 2,5-dioxopyrrolidin-1-yl 1-(trifluoromethyl)cyclopropanecarboxylate (3.600 g, 14.43 mmol) in tetrahydrofuran (40 mL) at 0° C. The resulting mixture was stirred at room temperature for 4 hours. TLC showed the reaction was complete. The reaction mixture was acidified with hydrochloric acid (2N) to pH 6-7, and extracted with dichloromethane-isopropyl alcohol (3:1 V/V, 40 mL×3). The combined organic layers were dried over anhydrous sodium sulfate and concentrated to give a crude residue which was purified through column chromatography using a 10-30% methanol/dichloromethane gradient to afford (S)-6-benzyl-2-(1-(trifluoromethyl)cyclopropanecarbonyl)-2,6-diazaspiro[3.4]octane-8-carboxylic acid (4.500 g, 81% yield) as a white solid. MS: [MH]⁺ 383.80.

(S)-2-(1-(trifluoromethyl)cyclopropane-1-carbonyl)-2,6-diazaspiro[3.4]octane-8-carboxylic acid: A mixture of (S)-6-benzyl-2-(1-(trifluoromethyl)cyclopropanecarbonyl)-2,6-diazaspiro[3.4]octane-8-carboxylic acid (2.000 g, 5.23 mmol) and Pd/C (0.200 g) in methanol (100 mL) was stirred at room temperature under hydrogen atomsphere overnight. Palladium on carbon was removed through filtration and washed with methanol (25 mL×2). The combined filtrates were concentrated to afford (S)-2-(1-(trifluoromethyl)cyclopropane-1-carbonyl)-2,6-diazaspiro[3.4]octane-8-carboxylic acid (1.530 g, 100%) as a white solid which was used in next step without further purification.

(S)-6-(1-(4-fluorobenzyl)-1H-pyrazole-4-carbonyl)-2-(1-(trifluoromethyl)cyclopropanecarbonyl)-2,6-diazaspiro[3.4]octane-8-carboxylic acid: To a mixture of (S)-2-(1-(trifluoromethyl)cyclopropanecarbonyl)-2,6-diazaspiro[3.4]octane-8-carboxylic acid (1.1 g, 3.77 mmol) and sodium bicarbonate in tetrahydrofuran-water (8 mL/8 mL) was added 2,5-dioxopyrrolidin-1-yl 1-(4-fluorobenzyl)-1H-pyrazole-4-carboxylate (1.20 g, 3.77 mmol) at 0-5° C. After stirring at room temperature for 15 hours, the resulting mixture was extracted with ethyl acetate (80 mL), washed with water (30 mL) and brine (20 mL), dried over anhydrous sodium sulfate, and concentrated under reduced pressure to give a crude which was purified through column chromatography using a 3% methanol in dichloromethane gradient to afford (S)-6-(1-(4-fluorobenzyl)-1H-pyrazole-4-carbonyl)-2-(1-(trifluoromethyl)cyclopropane carbonyl)-2,6-diazaspiro[3.4]octane-8-carboxylic acid (0.600 g, 38%) as a white solid. MS: [MH]⁺495.1.

(1-(4-Fluorobenzyl)-1H-pyrazol-4-yl)(8-(hydroxymethyl)-2-(1-(trifluoromethyl)cyclopropanecarbonyl)-2,6-diazaspiro[3.4]octan-6-yl)methanone: To a mixture of (S)-6-(1-(4-fluorobenzyl)-1H-pyrazole-4-carbonyl)-2-(1-(trifluoromethyl) cyclopropanecarbonyl)-2,6-diazaspiro[3.4]octane-8-carboxylic acid (0.595 g, 1.24 mmol) and 4-Methylmorpholine (0.137 g, 1.36 mmol) in anhydrous tetrahydrofuran (10 mL) at −20° C. was added isobutyl carbonochloridate (0.186 g, 1.36 mmol) in anhydrous tetrahydrofuran (2 mL) under nitrogen atmosphere, the resulting mixture was stirring at −20° C. under nitrogen atmosphere for 1 hour, followed by addition of sodium borohydride (0.094 g, 2.48 mmol) at 0-5° C. The mixture was stirred at room temperature for 2 hours. The reaction mixture was poured into ice water (30 mL), and extracted with ethyl acetate (80 mL). The organic layer was collected and washed with water (30 mL), brine (20 mL), dried over anhydrous sodium sulfate, and concentrated under reduced pressure to give a crude which was purified by column chromatography using a 3% methanol in dichloromethane gradient to afford (1-(4-fluorobenzyl)-1H-pyrazol-4-yl)(8-(hydroxymethyl)-2-(1-(trifluoromethyl)cyclopropanecarbonyl)-2,6-diazaspiro[3.4]octan-6-yl)methanone (0.250 g, 42%) as a white solid. MS: [MH]⁺ 481.2.

Methyl (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetate: A mixture of (S)-(1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (see i.e., I′-54) (0.120 g, 0.25 mmol) and sodium hydride (0.080 g, 2.00 mmol, 60% in mineral oil) in N,N-dimethylformamide (5 mL) was stirred at 0° C. under nitrogen atomsphere for 2 hours, followed by the addition of methyl 2-(2-(bromomethyl)-6-(4-(trifluoromethyl)cyclohexyl)phenyl)acetate (0.588 g, 1.50 mmol) in N,N-dimethylformamide (0.5 mL) at 0° C., the mixture was stirred at 0° C. for 10 mins. TLC showed the reaction was complete. The reaction mixture was poured into ice water (10 mL), and extracted with ethyl acetate (10 mL×2). The combined organic layers were washed with brine (5 mL), dried over anhydrous sodium sulfate, and concentrated to give a crude residue which was purified through silica gel flash column chromatography using a 5% methanol in dichloromethane gradient to afford methyl (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetate (0.100 g, 50%) as a yellow oil. MS: [MH]⁺ 793.3.

(S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetic acid: A mixture of methyl (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetate (0.100 g, 0.12 mmol) and lithium hydroxide monohydrate (0.012 g, 0.30 mmol) in tetrahydrofuran (4 mL)—methanol (2 mL)—water (2 mL) was stirred at 40° cfor 3 hours. TLC showed the reaction was complete. The reaction mixture was basified by hydrochloric acid (1 N) to pH 7 and extracted with ethyl acetate (10 mL). The organic layer was collected, and the aqueous layer was extracted with ethyl acetate (10 mL×2). The combined organic layers were concentrated to give residue which was purified by prep-HPLC to afford (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetic acid I-102 (0.007 g, yield 11%) as a white solid. ¹HNMR (400 MHz, CD₃OD): δ 8.18 (d, J=4.8 Hz, 1H), 7.89 (s, 1H), 7.32-7.29 (m, 2H), 7.23-7.17 (m, 2H), 7.12-7.05 (m, 3H), 4.57 (s, 2H), 4.30-4.18 (m, 1H), 4.08-3.97 (m, 1H), 3.94-3.87 (m, 2H), 3.81-3.79 (m, 2H), 3.73-3.57 (m, 3H), 2.88-2.36 (m, 3H), 2.11-2.02 (m, 3H), 1.78-1.43 (m, 6H), 1.18-1.05 (m, 5H), 0.88-0.83 (m, 3H). MS: [MH]⁺ 779.40.

The following compounds were prepared in a manner analogous to the procedures described above for (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenyl)acetic acid I-102:

(S)-2-(2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4-(trifluoromethyl)cyclohexyl)phenyl)acetic acid I′-23 (0.016 g, yield 65%) as a pink solid. ¹HNMR (400 MHz, MeOD): δ 8.21 (d, J=6.0 Hz, 1H), 7.91 (s, 1H), 7.35-7.32 (m, 2H), 7.24-7.09 (m, 5H), 5.36 (s, 2H), 4.61-4.58 (m, 2H), 4.40-4.21 (m, 1H), 4.09-3.88 (m, 3H), 3.83-3.81 (m, 2H), 3.76-3.59 (m, 4H), 3.51-3.49 (m, 1H), 3.16-3.14 (m, 1H), 2.89-2.80 (m, 1H), 2.67-2.37 (m, 2H), 2.14-2.03 (m, 2H), 1.83-1.61 (m, 5H), 1.43-1.34 (m, 7H). MS: [MH]⁺ 781.50.

### Synthesis of (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid I-101

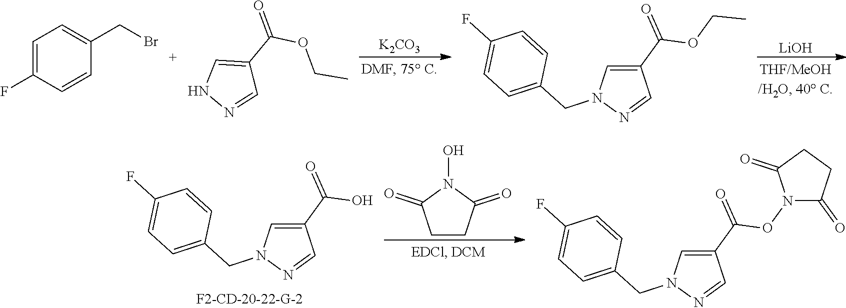

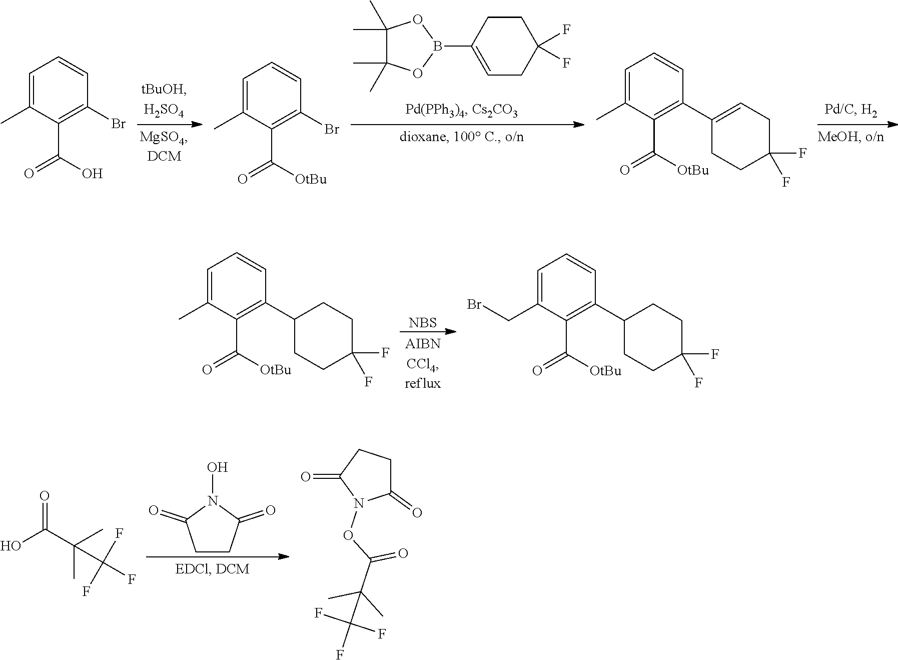

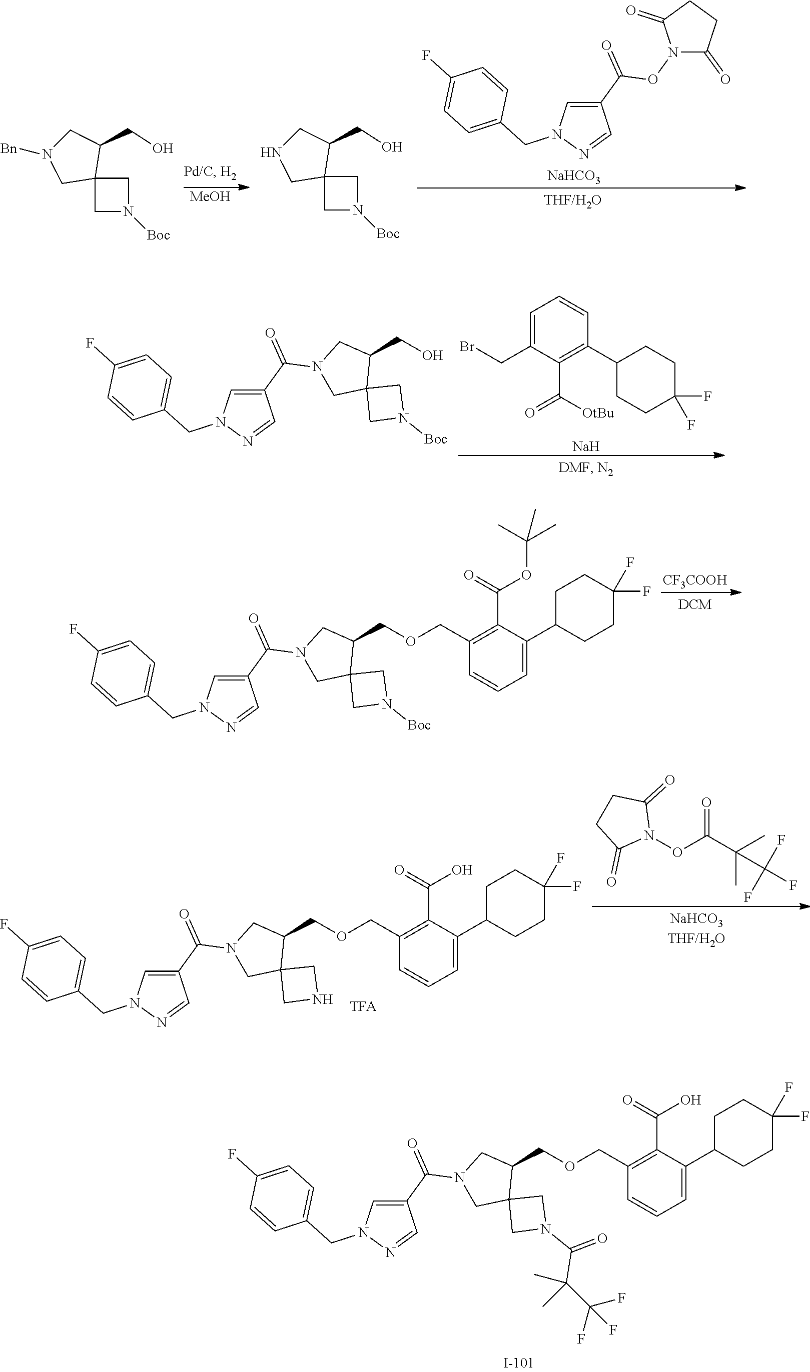

Ethyl 1-(4-fluorobenzyl)-1H-pyrazole-4-carboxylate: To a solution of 1-(bromomethyl)-4-fluorobenzene (5.0 g, 26.45 mmol) in N,N-dimethylformamide (20 mL) was added ethyl 1H-pyrazole-4-carboxylate (3.71 g, 26.45 mmol) and potassium carbonate (7.31 g, 52.90 mmol) under nitrogen atmosphere. The resulting mixture was stirred at 75° C. for 12 h. The reaction mixture was quenched with water, then diluted with ethyl acetate, washed with water, brine, dried over anhydrous sodium sulfate, and concentrated under reduced pressure to give a crude residue which was purified by column chromatography using a 25% ethyl acetate in hexane gradient to afford ethyl 1-(4-fluorobenzyl)-1H-pyrazole-4-carboxylate (5.97 g, 76%) as a white solid. MS: [MH]⁺ 249.20.

1-(4-Fluorobenzyl)-1H-pyrazole-4-carboxylic acid: To a solution of ethyl 1-(4-fluorobenzyl)-1H-pyrazole-4-carboxylate (5.97 g, 24.05 mmol) in water (5 mL)—methanol (10 mL)—tetrahydrofuran (20 mL) was added lithium hydroxide monohydrate (2.02 g, 48.10 mmol). The mixture was stirred at 40° C. for 5 h. The reaction mixture was concentrated to remove organic solvent, diluted with water, and ajusted pH to 3-5 with hydrochloric acid (2 M) at 0° C. The resulting mixture was extracted with ethyl acetate, washed with water and brine, dried over anhydrous sodium sulfate, and concentrated under reduced pressure to give a crude residue which was purified by column chromatography using a 2% methanol in dichloromethane gradient to afford 1-(4-fluorobenzyl)-1H-pyrazole-4-carboxylic acid (4.066 g, 77%) as a white solid. MS: [MH]+220.95.

2,5-Dioxopyrrolidin-1-yl 1-(4-fluorobenzyl)-1H-pyrazole-4-carboxylate: To a mixture of 1-(4-fluorobenzyl)-1H-pyrazole-4-carboxylic acid (1.000 g, 4.54 mmol) in dichloromethane (30 mL) at 0-5° C. was added 1-hydroxypyrrolidine-2,5-dione (0.627 g, 5.45 mmol) and N-(3-dimethylaminopyopyl)-N′-ethylcarbodiimide hydrochloride (1.130 g, 5.90 mmol). The resulting mixture was stirred at room temperature for 2 hours. The reaction mixture was concentrated under reduced pressure to give a crude residue which was purified by column chromatography using a 50% ethyl acetate in hexane gradient to afford 2,5-dioxopyrrolidin-1-yl 1-(4-fluorobenzyl)-1H-pyrazole-4-carboxylate (1.340 g, 93%) as a white solid. MS: [MH]+318.05.

Tert-butyl 2-bromo-6-methylbenzoate: To a solution of 2-bromo-6-methylbenzoic acid (15.5 g, 72.08 mmol) in dichloromethane (120 ml) was added magnesium sulfate (34.71 g, 288.32 mmol), concentrated sulfuric acid (7.11 g, 72.08 mmol), and tert-butanol (26.70 g, 360.4 mmol) under nitrogen atmosphere. The resulting mixture was stirred at room temperature overnight. The reaction mixture was quenched with water, extracted with ethyl acetate, washed with water and brine, dried over anhydrous sodium sulfate, and concentrated under reduced pressure to give a crude residue which was purified by column chromatography using a 100% hexane gradient to afford tert-butyl 2-bromo-6-methylbenzoate (16.01 g, 82%) as a yellow oil. ¹HNMR (400 MHz, CDCl₃): δ 7.38-7.33 (m, 1H), 7.13-7.10 (m, 2H), 2.35 (s, 3H), 1.62 (s, 9H).

Tert-butyl 4′,4′-difluoro-3-methyl-2′,3′,4′,5′-tetrahydro-[1,1′-biphenyl]-2-carboxylate: To a solution of tert-butyl 2-bromo-6-methylbenzoate (14.86 g, 54.80 mmol) in dioxane (150 mL) was added 2-(4,4-difluorocyclohex-1-en-1-yl)-4,4,5,5-tetramethyl-1,3,2-dioxaborolane (13.38 g, 54.80 mmol), cesium carbonate (26.78 g, 82.20 mmol), and tetrakis(triphenylphosphine)palladium (3.17 g, 2.74 mmol) under nitrogen atmosphere. The mixture was stirred at 100° C. overnight. The reaction mixture was cooled to room temperature, and concentrated to remove organic solvent under reduced pressure to give a crude residue which was purified by column chromatography using a 100% hexane gradient to afford tert-butyl 4′,4′-difluoro-3-methyl-2′,3′,4′,5′-tetrahydro-[1,1′-biphenyl]-2-carboxylate (17.06 g, crude) as a yellow oil. ¹HNMR (400 MHz, CDCl₃): δ 7.15 (t, J 7.6 Hz, 1H), 7.05-7.02 (m, 1H), 6.93 (d, J=7.6 Hz, 1H), 5.43-5.40 (m, 1H), 2.59-2.52 (m, 4H), 2.28 (s, 3H), 2.13-2.02 (m, 2H), 1.46 (s, 9H).

Tert-butyl 2-(4,4-difluorocyclohexyl)-6-methylbenzoate: To a solution of tert-butyl 4′,4′-difluoro-3-methyl-2′,3′,4′,5′-tetrahydro-[1,1′-biphenyl]-2-carboxylate (17.06 g, 55.97 mmol) in methanol (100 mL) was added Pd/C (3.45 g) under hydrogen atmosphere. The resulting mixture was stirred at room temperature overnight. The reaction mixture was filtered and concentrated under reduced pressure to tert-butyl 2-(4,4-difluorocyclohexyl)-6-methylbenzoate (14.12 g, 81%) as a white solid. ¹HNMR (400 MHz, CDCl₃): δ 7.24-7.22 (m, 1H), 7.11 (d, J=8.0 Hz, 1H), 7.05 (d, J=7.6 Hz, 1H), 2.68-2.62 (m, 1H), 2.33 (s, 3H), 2.24-2.19 (m, 2H), 1.97-1.95 (m, 2H), 1.84-1.74 (m, 4H), 1.61 (s, 9H).

Tert-butyl 2-(bromomethyl)-6-(4,4-difluorocyclohexyl)benzoate: To a solution of tert-butyl 2-(4,4-difluorocyclohexyl)-6-methylbenzoate (14.120 g, 45.49 mmol) in carbon tetrachloride (150 mL) was added N-bromosuccinimide (9.720 g, 54.59 mmol) and 2,2′-azobis(2-methylpropionitrile) (0.747 g, 4.55 mmol) under nitrogen atmosphere. The resulting mixture was stirred at 90° C. overnight. The reaction mixture was concentrated under reduced pressure to give a crude residue which was purified by column chromatography using a 2% ethyl acetate in hexane gradient to afford tert-butyl 2-(bromomethyl)-6-(4,4-difluorocyclohexyl)benzoate (9.160 g, 51%) as a white solid. ¹HNMR (400 MHz, CDCl₃): δ 7.29-7.17 (m, 3H), 4.46 (s, 2H), 2.71-2.62 (m, 1H), 2.19-2.12 (m, 2H), 1.91-1.88 (m, 2H), 1.77-1.74 (m, 2H), 1.71-1.67 (m, 2H), 1.58 (s, 9H).

2,5-Dioxopyrrolidin-1-yl 3,3,3-trifluoro-2,2-dimethylpropanoate: A mixture of 3,3,3-trifluoro-2,2-dimethylpropanoic acid (0.100 g, 0.64 mmol), 1-hydroxypyrrolidine-2,5-dione (0.074 g, 0.64 mmol), and EDCI (147.4 mg, 0.77 mmol) in dichloromethane (2 mL) was stirred at room temperature. The mixture was quenched with water (15 mL), extracted with ethyl acetate, dried with sodium sulfate, and concentrated to afford 2,5-dioxopyrrolidin-1-yl3,3,3-trifluoro-2,2-dimethylpropanoate as a white solid (0.154 g, 95%). ¹HNMR (400 MHz, CDCl₃): δ 2.85 (s, 4H), 1.62 (s, 6H).

Tert-butyl (S)-8-(hydroxymethyl)-2,6-diazaspiro[3.4]octane-2-carboxylate: A mixture of tert-butyl (S)-6-benzyl-8-(hydroxymethyl)-2,6-diazaspiro[3.4]octane-2-carboxylate (2.000 g, 6.02 mmol) and Pd/C (0.200 g) in methanol (100 mL) was stirred at room temperature under hydrogen atomsphere overnight. Palladium on carbon was removed through filtration and washed with methanol (25 mL×2). The combined filtrates were concentrated to afford tert-butyl (S)-8-(hydroxymethyl)-2,6-diazaspiro[3.4]octane-2-carboxylate (1.450 g, 100%) as a white solid which was used in next step without further purification.

Tert-butyl (S)-6-(1-(4-fluorobenzyl)-1H-pyrazole-4-carbonyl)-8-(hydroxy methyl)-2,6-diazaspiro[3.4]octane-2-carboxylate: To a solution of tert-butyl (S)-8-(hydroxymethyl)-2,6-diazaspiro[3.4]octane-2-carboxylate (1.450 g, 6.00 mmol) in water (50 mL) was added sodium bicarbonate (2.520 g, 30.00 mmol), followed by addition of 2,5-dioxopyrrolidin-1-yl 1-(4-fluorobenzyl)-1H-pyrazole-4-carboxylate (2.871 g, 9.00 mmol) in tetrahydrofuran (15 mL), the resulting mixture was stirred at room temperature for 4 hours. TLC showed the reaction was complete. The reaction mixture was extracted with ethyl acetate (30 mL×3). The combined organic phases were washed with brine (30 mL×2), dried over anhydrous sodium sulfate, and concentrated under reduced pressure to give a crude residue which was purified by column chromatography using 1% methanol in dichloromethane gradient to afford tert-butyl (S)-6-(1-(4-fluorobenzyl)-1H-pyrazole-4-carbonyl)-8-(hydroxymethyl)-2,6-diazaspiro[3.4]octane-2-carboxylate (1.864 g, 70% yield) as colorless oil. MS: [MH]⁺ 445.40.

(S)-tert-butyl 8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate: To a stirred solution of tert-butyl (S)-6-(1-(4-fluorobenzyl)-1H-pyrazole-4-carbonyl)-8-(hydroxymethyl)-2,6-diazaspiro[3.4]octane-2-carboxylate (1.500 g, 3.38 mmol) in anhydrous THF (50 mL) at 0° C. was added sodium hydride (60% in mineral oil) (0.405 g, 10.13 mmol). The resulting mixture was stirred at room temperature for 1 hour, followed by addition of tert-butyl 2-(bromomethyl)-6-(4,4-difluorocyclohexyl)benzoate (3.934 g, 10.14 mmol) in anhydrous THE (20 mL), and the resulting mixture was stirred at room temperature overnight. TLC showed the reaction was complete. The reaction mixture was poured into water (50 mL) and extracted with ethyl acetate (50 mL×2). The combined organic layer was washed with water (30 mL×3) and brine (10 mL), dried over anhydrous sodium sulfate, and concentrated under reduced pressure to give a crude residue which was column chromatography using 5% methanol in dichloromethane gradient to afford (S)-tert-butyl 8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate (0.985 g, 47% yeild) as a white solid. MS: [MH]⁺ 621.50.

(S)-2-(4,4-difluorocyclohexyl)-6-(((6-(1-(4-fluorobenzyl)-1H-pyrazole-4-carbonyl)-2,6-diazaspiro[3.4]octan-8-yl)methoxy)methyl)benzoic acid TFA salt: A mixture of (S)-tert-butyl 8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate (0.985 g, 1.58 mmol) and TFA (5 mL) in dichloromethane (10 mL) was stirred at room temperature overnight. TLC showed the reaction was complete. The volatiles were evaporated under reduced pressure to afford (S)-2-(4,4-difluorocyclohexyl)-6-(((6-(1-(4-fluorobenzyl)-1H-pyrazole-4-carbonyl)-2,6-diazaspiro[3.4]octan-8-yl)methoxy)methyl)benzoic acid TFA salt (1.100 g, 100%) as a light yellow solid which was used in next step without further purification. MS: [MH]⁺ 597.10.

(S)-2-(4, 4-difluorocyclohexyl)-6-(((6-(1-(4-fluorobenzyl)-1H-pyrazole-4-carbonyl)-2-(3,3,3-trifluoro-2,2-dimethylpropanoyl)-2, 6-diazaspiro[3.4]octan-8-yl)methoxy)methyl)benzoic acid I-101: To a solution of S)-2-(4,4-difluorocyclohexyl)-6-(((6-(1-(4-fluorobenzyl)-1H-pyrazole-4-carbonyl)-2,6-diazaspiro[3.4]octan-8-yl)methoxy)methyl)benzoic acid TFA salt (0.100 g, 0.14 mmol) and sodium bicarbonate (0.059 g, 0.70 mmol) in water (2 mL) was added 2,5-dioxopyrrolidin-1-yl 3,3,3-trifluoro-2,2-dimethylpropanoate (0.071 g, 0.28 mmol) in tetrahydrofuran (4 mL). The resulting mixture was stirred at room temperature for 10 hours. The reaction mixture was acidified with hydrochloric acid (2 M) to pH 4-5, extracted with ethyl acetate (10 mL*3), dried with sodium sulfate, and concentrated to give a residue which was purified through column chromatography by using 3% dichloromethane in methanol gradient to afford (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid as a white solid (0.025 g, 12%). ¹HNMR (400 MHz, CD₃OD): δ 8.24 (d, J=33.2 Hz, 1H), 7.91 (d, J=6.4 Hz, 1H), 7.32 (t, J=4.0 Hz, 2H), 7.27-7.26 (m, 2H), 7.20-7.18 (m, 1H), 7.08-7.06 (m, 2H), 5.35 (s, 2H), 4.59 (s, 2H), 4.06-3.67 (m, 8H), 2.86 (s, 1H), 2.60-2.55 (m, 1H), 2.10 (s, 2H), 1.91-1.85 (m, 3H), 1.79-1.77 (m, 3H), 1.38-1.20 (m, 6H). MS: [MH]⁺ 735.35.

The following compounds were prepared in a manner analogous to the procedures described above for (S)-2-(4,4-difluorocyclohexyl)-6-(((6-(1-(4-fluorobenzyl)-JH-pyrazole-4-carbonyl)-2-(3,3,3-trifluoro-2,2-dimethylpropanoyl)-2,6-diazaspiro[3.4]octan-8-yl)methoxy)methyl)benzoic acid (I-101):

(S)-2-(((2-(bicyclo[11.1.1]pentane-1-carbonyl)-6-(1-(4-fluorobenzyl)-1H-pyrazole-4-carbonyl)-2,6-diazaspiro[3.4]octan-8-yl)methoxy)methyl)-6-(4,4-difluorocyclohexyl)benzoic acid I′-17 (0.055 g, yield 14%) as a white solid. 1HNMR (400 MHz, CD₃OD): δ 8.23 (dd, J₁=25.6 Hz, J₂=3.6 Hz, 1H), 7.92-7.89 (m, 1H), 7.34-7.31 (m, 4H), 7.25-7.20 (m, 1H), 7.10-7.04 (m, 2H), 5.35 (s, 2H), 4.61-4.54 (m, 2H), 4.41-3.44 (m, 10H), 2.87-2.56 (m, 2H), 2.41-2.37 (m, 1H), 2.14-2.01 (m, 8H), 1.93-1.75 (m, 6H). MS: [MH]⁺ 691.25.

(S)-2-(((2-(3,3-difluoro-2,2-dimethylpropanoyl)-6-(1-(4-fluorobenzyl)-1H-pyrazole-4-carbonyl)-2,6-diazaspiro[3.4]octan-8-yl)methoxy)methyl)-6-(4,4-difluorocyclohexyl)benzoic acid I′-8 (0.007 g, yield 15%) as a white solid. ¹H NMR (400 MHz, CD₃OD): δ 8.30-8.23 (m, 1H), 7.94 (d, J=9.6 Hz, 1H), 7.38-7.23 (m, 5H), 7.12-7.07 (m, 2H), 6.18-5.87 (m, 1H), 5.38 (s, 2H), 4.73-4.22 (m, 4H), 4.10-3.50 (m, 7H), 3.15 (s, 1H), 2.87-2.82 (m, 1H), 2.69-2.63 (m, 1H), 2.14-2.06 (m, 2H), 1.96-1.80 (m, 6H), 1.24-1.15 (m, 6H). MS: [MH]⁺ 717.25.

(S)-2-(4,4-difluorocyclohexyl)-6-(((6-(1-(4-fluorobenzyl)-1H-pyrazole-4-carbonyl)-2-(3,3,3-trifluoropropanoyl)-2,6-diazaspiro[3.4]octan-8-yl)methoxy)methyl)benzoic acid I′-7 (0.093 g, yield 18%) as a white solid. ¹HNMR (400 MHz, CD₃OD): δ 8.27-8.19 (m, 1H), 7.92-7.88 (m, 1H), 7.34-7.30 (m, 4H), 7.23-7.20 (m, 1H), 7.10-7.04 (m, 2H), 5.35 (s, 2H), 4.63-4.47 (m, 2H), 4.30-3.47 (m, 10H), 3.23-2.97 (m, 2H), 2.88-2.77 (m, 1H), 2.73-2.56 (m, 1H), 2.17-2.04 (m, 2H), 1.93-1.71 (m, 6H). MS: [MH]⁺ 707.25.

(S)-2-(4,4-difluorocyclohexyl)-6-(((6-(1-(4-fluorobenzyl)-1H-pyrazole-4-carbonyl)-2-isobutyryl-2,6-diazaspiro[3.4]octan-8-yl)methoxy)methyl)benzoic acid I′-16 (0.062 g, yield 14%) as a white solid. ¹HNMR (400 MHz, CD₃OD): δ 8.26-8.19 (m, 1H), 7.92-7.89 (m, 1H), 7.33-7.30 (m, 4H), 7.24-7.20 (m, 1H), 7.10-7.04 (m, 2H), 5.35-5.34 (m, 2H), 4.60-4.57 (m, 2H), 4.46-3.48 (m, 10H), 2.90-2.78 (m, 1H), 2.73-2.42 (m, 2H), 2.16-2.11 (m, 2H), 1.93-1.77 (m, 6H), 1.07-0.96 (m, 6H). MS: [MH]⁺ 667.35.

(S)-2-(4,4-difluorocyclohexyl)-6-(((6-(1-(4-fluorobenzyl)-1H-pyrazole-4-carbonyl)-2-pivaloyl-2,6-diazaspiro[3.4]octan-8-yl)methoxy)methyl)benzoic acid I′-6 (0.010 g, yield 17%) as a white solid. ¹H NMR (400 MHz, CD3OD): δ 8.30-8.23 (m, 1H), 7.95-7.93 (m, 1H), 7.35-7.10 (m, 7H), 5.38 (s, 2H), 4.67-3.50 (m, 9H), 4.10-3.50 (m, 7H), 3.15 (s, 1H), 2.87-2.59 (m, 3H), 2.13-2.06 (m, 8H), 1.318 (s, 2H), 1.16-1.14 (m, 9H). MS: [MH]⁺ 681.35.

(S)-2-(4,4-difluorocyclohexyl)-6-(((6-(1-(4-fluorobenzyl)-1H-pyrazole-4-carbonyl)-2-(1-(trifluoromethyl)cyclopentane-1-carbonyl)-2,6-diazaspiro[3.4]octan-8-yl)methoxy)methyl)benzoic acid I′-5 (0.005 g, yield 10%) as a white solid. ¹HNMR (400 MHz, CD₃OD): δ 8.27-8.20 (m, 1H), 7.92-7.89 (m, 1H), 7.37-7.30 (m, 4H), 7.24-7.20 (m, 1H), 7.09-7.04 (m, 2H), 5.35 (s, 2H), 4.63-4.59 (m, 2H), 4.49-3.46 (m, 10H), 2.89-2.80 (m, 1H), 2.67-2.56 (m, 1H), 2.32-1.55 (m, 16H). MS: [MH]⁺ 761.25.

(S)-2-(4,4-difluorocyclohexyl)-6-(((6-(1-(4-fluorobenzyl)-1H-pyrazole-4-carbonyl)-2-(1-(2-fluorophenyl)cyclopropane-1-carbonyl)-2,6-diazaspiro[3.4]octan-8-yl)methoxy)methyl)benzoic acid I′-4 as a white solid (0.021 g, 28%) ¹H NMR (400 MHz, DMSO): δ 8.26-8.18 (m, 1H), 7.90-7.87 (m, 1H), 7.37-7.23 (m, 7H), 7.13-7.00 (m, 4H), 5.36 (s, 2H), 4.58-4.53 (m, 2H), 4.19-3.54 (m, 8H), 2.93-2.85 (m, 1H), 2.60-2.42 (m, 1H), 2.22-1.74 (m, 9H), 1.59-1.47 (m, 2H), 1.32-1.11 (m, 4H) MS: [MH]⁺ 759.35.

(S)-2-(4,4-difluorocyclohexyl)-6-(((6-(1-(4-fluorobenzyl)-1H-pyrazole-4-carbonyl)-2-(1-(4-fluorophenyl)cyclopropane-1-carbonyl)-2,6-diazaspiro[3.4]octan-8-yl)methoxy)methyl)benzoic acid I′-3 (0.007 g, yield 20%) as a white solid. ¹H NMR (400 MHz, CD₃OD): δ 8.24-8.16 (m, 1H), 7.88-7.86 (m, 1H), 7.38-7.23 (m, 7H), 7.10-6.95 (m, 4H), 5.34 (s, 2H), 4.57-4.52 (m, 2H), 3.96-3.39 (m, 9H), 2.87 (s, 1H), 2.55-2.41 (m, 1H), 2.13-1.80 (m, 8H), 1.43-1.36 (m, 2H), 1.11-1.00 (m, 2H). MS: [MH]⁺ 759.00.

(S)-2-(4,4-difluorocyclohexyl)-6-(((6-(1-(4-fluorobenzyl)-1H-pyrazole-4-carbonyl)-2-(1-fluorocyclohexane-1-carbonyl)-2,6-diazaspiro[3.4]octan-8-yl)methoxy)methyl)benzoic acid I′-2 (0.016 g, 16%) as a white solid. ¹HNMR (400 MHz, CD₃OD): δ 8.29-8.22 (m, 1H), 7.94-7.92 (m, 1H), 7.36-7.32 (m, 4H), 7.27-7.23 (m, 1H), 7.12-7.06 (m, 2H), 5.37 (s, 2H), 4.66-4.57 (m, 2H), 4.53-4.48 (m, 1H), 4.29-4.23 (m, 1H), 4.10-4.03 (m, 1H), 4.00-3.88 (m, 2H), 3.84-3.78 (m, 1H), 3.76-3.62 (m, 3H), 3.52-3.49 (m, 1H), 2.87-2.83 (m, 1H), 2.71-2.61 (m, 1H), 2.13-2.12 (m, 2H), 1.92-1.73 (m, 10H), 1.71-1.55 (m, 6H), 1.31 (s, 1H). MS: [MH]⁺ 725.40.

(S)-2-(4,4-difluorocyclohexyl)-6-(((6-(1-(4-fluorobenzyl)-1H-pyrazole-4-carbonyl)-2-(3-methyloxetane-3-carbonyl)-2,6-diazaspiro[3.4]octan-8-yl)methoxy)methyl)benzoic acid I′-1 (0.010 g, 21% yeild) as a white solid. 1¹HNMR (400 MHz, CDCl₃): δ 7.90-7.89 (m, 1H), 7.79 (s, 1H), 7.33 (s, 2H), 7.24-7.20 (m, 2H), 7.13-7.12 (m, 1H), 7.04 (t, J=16.4 Hz, 2H), 5.27 (s, 2H), 5.03-4.82 (m, 3H), 4.43-4.22 (m, 4H), 4.07-3.97 (m, 2H), 3.88-3.78 (m, 2H), 3.72-3.58 (m, 3H), 3.42-3.22 (m, 5H), 2.92-2.86 (m, 1H), 2.67-2.53 (m, 1H), 2.21-1.73 (m, 8H). MS: [MH]⁺ 785.5.

### Synthesis of (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenoxy)acetic acid I-109

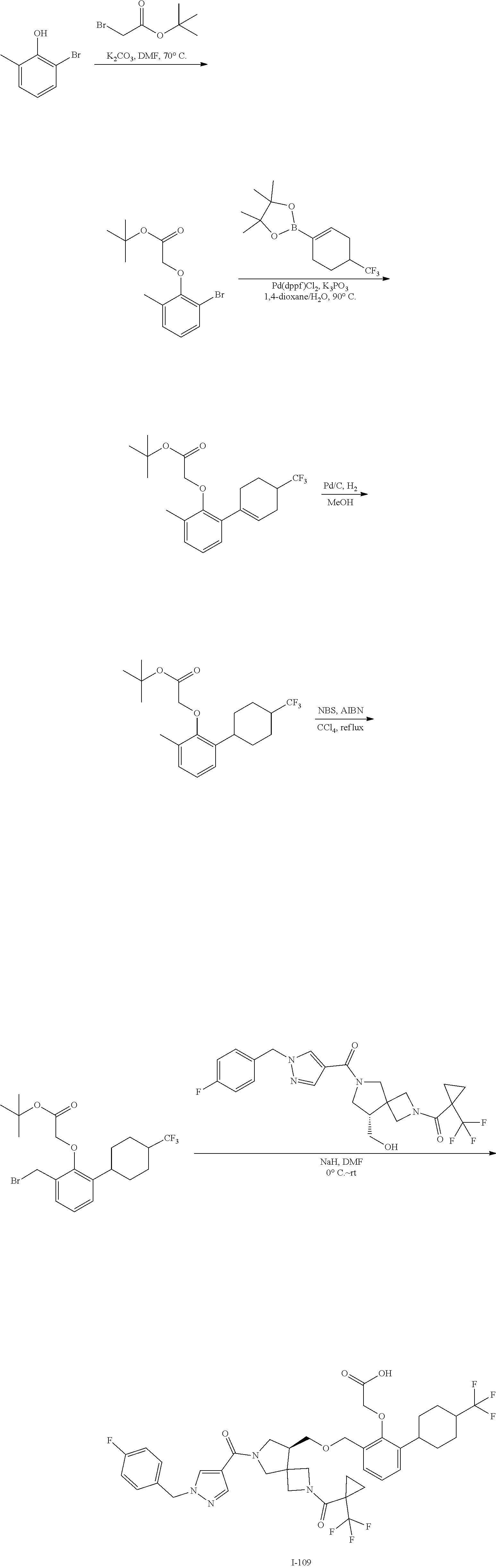

Tert-butyl 2-(2-bromo-6-methylphenoxy)acetate: A mixture of 2-bromo-6-methylphenol (1.000 g, 5.40 mmol), tert-butyl 2-bromoacetate (2.100 g, 10.70 mmol) and potassium carbonate (1.800 g, 13.40 mmol) in N,N-dimethylformamide (20 mL) was stirred at 70° C. under nitrogen atmosphere overnight. TLC showed the reaction was completed. The reaction mixture was cooled to room temperature, poured into water (10 mL), and extracted with ethyl acetate (10 mL). The organic layer was collected, and the aqueous layer was extracted with ethyl acetate (10 mL×2). The combined organic layers were washed with brine (10 mL), dried over anhydrous sodium sulfate and concentrated to give a crude residue which was purified by column chromatography by using 1% ethyl acetate in hexane gradient to afford tert-butyl 2-(2-bromo-6-methylphenoxy)acetate (1.400 g, 87% yield) as a colorless oil. ¹HNMR (400 MHz, CDCl₃): δ 7.36 (d, J=7.6 Hz, 1H), 7.11 (d, J=7.6 Hz, 1H), 6.90 (t, J=7.6 Hz, 1H), 4.34 (s, 2H), 2.36 (s, 3H), 1.52 (s, 9H).

Tert-butyl 2-((3-methyl-4′-(trifluoromethyl)-2′,3′,4′,5′-tetrahydro-[1,1′-biphenyl]-2-yl)oxy)acetate: To a stirred solution of tert-butyl 2-(2-bromo-6-methylphenoxy)acetate (1.200 g, 4.00 mmol), 4,4,5,5-tetramethyl-2-(4-(trifluoromethyl)cyclohex-1-en-1-yl)-1,3,2-dioxaborolane (1.2 g, 4.4 mmol) and potassium phosphate (1.700 g, 8.00 mmol) in 1,4-dioxane (16 mL)—water (4 mL) was added 1,1′-Bis(diphenylphosphino)ferrocene palladium(II)dichloride (0.289 g, 0.40 mmol) at room temperature under nitrogen atmosphere; The resulting mixture was stirred at 90° C. under nitrogen atmosphere overnight. TLC showed the reaction was completed. The mixture was poured into water (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate and concentrated to give a crude residue which was purified by column chromatography by using a 1% ethyl acetate in hexane gradient to afford tert-butyl 2-((3-methyl-4′-(trifluoromethyl)-2′,3′,4′,5′-tetrahydro-[1,1′-biphenyl]-2-yl)oxy)acetate (1.200 g, 80% yield) as light yellow oil. ¹HNMR (400 MHz, CDCl₃): δ 7.10-7.05 (m, 1H), 7.01-6.92 (m, 2H), 5.82-5.76 (m, 1H), 4.23 (s, 2H), 2.62-2.36 (m, 4H), 2.32 (s, 3H), 2.28-2.19 (m, 1H), 2.15-2.08 (m, 1H), 1.72-1.64 (m, 1H), 1.50 (s, 9H).

Tert-butyl 2-(2-methyl-6-(4-(trifluoromethyl)cyclohexyl)phenoxy)acetate: A mixture of tert-butyl 2-((3-methyl-4′-(trifluoromethyl)-2′,3′,4′,5′-tetrahydro-[1,1′-biphenyl]-2-yl)oxy)acetate (1.200 g, 3.20 mmol), palladium on carbon (10%, 0.120 g) in methanol (25 mL) was stirred at room temperature under hydrogen atmosphere (hydrogen balloon) overnight. Palladium on carbon was removed through filtration and washed with methanol (10 mL×2). The filtrates were concentrated under reduced pressure to give a crude residue which was purified by column chromatography by using a 1% ethyl acetate in hexane gradient to afford tert-butyl 2-(2-methyl-6-(4-(trifluoromethyl)cyclohexyl)phenoxy)acetate (0.645 g, 53% yield) as a colorless oil. HNMR (400 MHz, CDCl₃): δ 7.10-7.05 (m, 1H), 7.03-6.99 (m, 2H), 4.27 (s, 2H), 3.18-3.08 (m, 1H), 2.43-2.32 (m, 1H), 2.30 (s, 3H), 2.13-2.04 (m, 2H), 1.79-1.62 (m, 6H), 1.53 (s, 9H).

Tert-butyl 2-(2-(bromomethyl)-6-(4-(trifluoromethyl)cyclohexyl) phenoxy)acetate: A mixture of tert-butyl 2-(2-methyl-6-(4-(trifluoromethyl)cyclohexyl)phenoxy)acetate (645 mg, 1.7 mmol), N-bromosuccinimide (308.6 mg, 1.7 mmol), and 2,2′-Azobis(2-methylpropionitrile) in carbon tetrachloride (10 mL) was reflux for 3 hours. TLC showed the reaction was completed. The resulting mixture was concentrated under reduced pressure to give a crude residue which was purified by column chromatography using a 1% ethyl acetate in hexane gradient to afford tert-butyl 2-(2-(bromomethyl)-6-(4-(trifluoromethyl)cyclohexyl)phenoxy)acetate (0.210 g; 26% yield) as a light yellow oil.

(S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenoxy)acetic acid I-109: To a solution of (S)-(1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (see i.e., I′-54) (0.120 g, 0.25 mmol) in N,N-dimethylformamide (4 mL) at 0-5° C. was added sodium hydride (60% in mineral oil) (0.080 g, 2.00 mmol), the mixture was stirred at room temperature for 30 minutes, followed by the addition of tert-butyl 2-(2-(bromomethyl)-6-(4-(trifluoromethyl)cyclohexyl)phenoxy)acetate (0.225 g, 0.50 mmol) in N,N-dimethylformamide (2 mL). The resulting mixture was stirred at room temperature under nitrogen atmosphere for 2 hours. TLC showed the reaction was completed. The mixture was poured into water (20 mL) and extracted with ethyl acetate (20 mL). The organic layer was collected, and the aqueous layer was extracted with ethyl acetate (10 mL×2). The combined organic layers were washed with brine (10 mL), dried over anhydrous sodium sulfate and concentrated to give a crude residue which was purified by Pre-HPLC to afford (S)-2-(2-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)phenoxy)acetic acid (0.033 g, 16% yield) as oil-solid. ¹HNMR (400 MHz, CD₃OD): δ 8.18 (s, 1H), 7.89 (s, 1H), 7.36-7.28 (m, 2H), 7.24-7.14 (m, 2H), 7.11-7.02 (m, 3H), 5.35 (s, 2H), 4.61-4.54 (m, 2H), 4.46 (d, J=7.6 Hz, 2H), 4.43-3.44 (m, 10H), 3.14-3.03 (m, 1H), 2.74-2.59 (m, 1H), 2.50-2.36 (m, 1H), 2.13-2.04 (m, 2H), 1.85-1.63 (m, 6H), 1.24-1.08 (m, 4H). MS. [MH]⁺ 795.30.

### Synthesis of 2′-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylic acid I-104

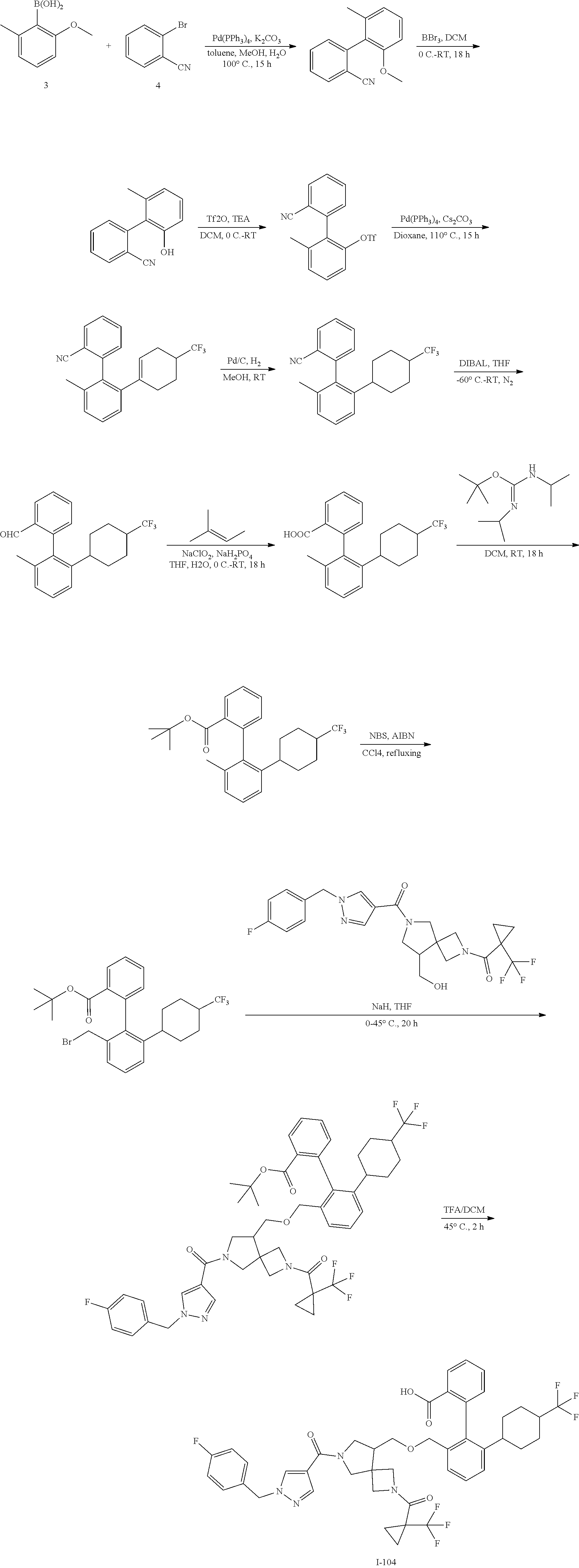

2′-Methoxy-6′-methyl-[1,1′-biphenyl]-2-carbonitrile: A mixture of (2-methoxy-6-methylphenyl)boronic acid (2.40 g, 14.45 mmol), 2-bromobenzonitrile (2.60 g, 14.45 mmol) and tetrakis(triphenylphosphine)palladium (940 mg, 0.81 mmol) in a mixture of toluene-ethanol-water (50 mL, 3:1:1) was stirred at 100° C. under nitrogen atmosphere for 15 hours. The resulting mixture was poured into ice water, extracted with ethyl acetate, washed with water, brine, dried over anhydrous sodium sulfate, and concentrated under reduced pressure to give a crude which was purified by column chromatography using a 2% ethyl acetate in hexane gradient to afford 2′-methoxy-6′-methyl-[1,1′-biphenyl]-2-carbonitrile (2.70 g, 84%) as a colorless oil. ¹HNMR (400 MHz, CDCl₃): δ 7.75 (d, J=7.6 Hz, 1H), 7.66-7.61 (m, 1H), 7.45-7.41 (m, 1H), 7.34-7.28 (m, 2H), 6.93 (d, J=7.6 Hz, 1H), 6.85 (d, J=8.4 Hz, 1H), 3.74 (s, 3H), 2.06 (s, 3H).

2′-Hydroxy-6′-methyl-[1,1′-biphenyl]-2-carbonitrile: To a mixture of 2′-methoxy-6′-methyl-[1,1′-biphenyl]-2-carbonitrile (2.70 g, 24.2 mmol) in dichloromethane (10 mL) at 0-5° C. was added boron tribromide (1 N in dichloromethane) (24.2 mL, 24.2 mmol) under nitrogen atmosphere. After stirring at room temperature for 18 hours, the reaction mixture was diluted with ice water, extracted with dichloromethane, washed with brine, dried over anhydrous sodium sulfate, and concentrated under reduced pressure to give a crude residue which was purified by column chromatography using a 10% ethyl acetate in hexane afford 2′-hydroxy-6′-methyl-[1,1′-biphenyl]-2-carbonitrile (2.45 g, 97%) as a white solid. ¹HNMR (400 MHz, CDCl₃): δ 7.81-7.79 (m, 1H), 7.71-7.67 (m, 1H), 7.52-7.48 (m, 1H), 7.40 (d, J=7.6 Hz, 1H), 7.20 (t, J=7.6 Hz, 1H), 6.90 (d, J=7.6 Hz, 1H), 6.77 (t, J=8.0 Hz, 1H), 4.87-4.85 (m, 1H), 2.07 (s, 3H).

2′-Cyano-6-methyl-[1,1′-biphenyl]-2-yl trifluoromethanesulfonate: To a mixture of 2′-hydroxy-6′-methyl-[1,1′-biphenyl]-2-carbonitrile (2.45 g, 11.72 mmol) and triethylamine (2.56 g, 23.44 mmol) in DCM at 0-5° C. was added trifluoromethanesulfonic anhydride (6.61 g, 23.44 mmol) under nitrogen atmosphere. The resulting mixture was stirred at room temperature under nitrogen atmosphere for 15 hours. The reaction mixture was diluted with ice water, extracted with dichloromethane, washed with brine, dried over anhydrous sodium sulfate, and concentrated under reduced pressure to give a crude residue which was purified by column chromatography using a 2% ethyl acetate in hexane afford 2′-hydroxy-6′-methyl-[1,1′-biphenyl]-2-carbonitrile (3.20 g, 80%) as a colorless oil. ¹HNMR (400 MHz, CDCl₃): δ 7.81 (d, J=7.6 Hz, 1H), 7.71 (t, J=7.6 Hz, 1H), 7.55 (t, J=7.6 Hz, 1H), 7.46-7.36 (m, 3H), 7.27 (d, J=7.6 Hz, 1H), 2.18 (s, 3H).

6′-Methyl-4″-(trifluoromethyl)-2″,3″,4″,5″-tetrahydro-[1,1′:2′,1″-terphenyl]-2-carbonitrile: A mixture of 2′-hydroxy-6′-methyl-[1,1′-biphenyl]-2-carbonitrile (3.20 g, 9.38 mmol), 4,4,5,5-tetramethyl-2-(4-(trifluoromethyl)cyclohex-1-en-1-yl)-1,3,2-dioxaborolane (2.59 g, 9.38 mmol), Tetrakis(triphenylphosphine)palladium (542 mg, 0.47 mmol) and Cesium carbonate (4.62 g, 14.07 mmol) in dioxane (60 mL) was stirred at 110° C. under nitrogen atmosphere for 15 hours. The mixture was extracted with ethyl acetate, washed with water, brine, dried over anhydrous sodium sulfate, and concentrated under reduced pressure to give a crude which was purified by column chromatography using a 2% ethyl acetate in hexane gradient to afford 6′-methyl-4″-(trifluoromethyl)-2″,3″,4″,5″-tetrahydro-[1,1′:2′,1″-terphenyl]-2-carbonitrile (2.50 g, 78%) as a yellow oil. ¹HNMR (400 MHz, CDCl₃): δ 7.72 (d, J=7.6 Hz, 1H), 7.60 (t, J=7.6 Hz, 1H), 7.43 (t, J=7.6 Hz, 1H), 7.31-7.21 (m, 3H), 7.08 (d, J=7.2 Hz, 1H), 5.51-5.46 (m, 1H), 2.19-2.16 (m, 1H), 2.07 (s, 3H), 2.04-1.83 (m, 5H), 1.36-1.22 (m, 1H).

2′-Methyl-6′-(4-(trifluoromethyl)cyclohexyl)-[1,1′-biphenyl]-2-carbonitrile: A mixture of 6′-methyl-4″-(trifluoromethyl)-2″,3″,4″,5″-tetrahydro-[1,1′:2′,1″-terphenyl]-2-carbonitrile and Palladium on carbon (10%, 360.0 mg) in methanol (25 mL) was stirred at 25° C. under hydrogen atmosphere (hydrogen balloon) overnight. Palladium on carbon was removed through filtration and washed with methanol (20 ml×2). The combined filtrate was concentrated under reduced pressure to give a crude which was purified by column chromatography using a 2% ethyl acetate in hexane gradient to afford 2′-methyl-6′-(4-(trifluoromethyl)cyclohexyl)-[1,1′-biphenyl]-2-carbonitrile (1.05 g, 40%) as a colorless oil. ¹HNMR (400 MHz, CDCl₃): δ 7.79-7.77 (m, 1H), 7.68-7.64 (m, 1H), 7.51-7.47 (m, 1H), 7.34-7.26 (m, 2H), 7.23 (d, J=8.0 Hz, 1H), 7.15 (d, J=7.2 Hz, 1H), 2.25-2.19 (m, 1H), 2.11-2.05 (m, 3H), 1.97 (s, 3H), 1.83-1.63 (m, 3H), 1.51-1.31 (m, 4H).

2′-Methyl-6′-(4-(trifluoromethyl)cyclohexyl)-[1,1′-biphenyl]-2-carbaldehyde: To a mixture of 2′-methyl-6′-(4-(trifluoromethyl)cyclohexyl)-[1,1′-biphenyl]-2-carbonitrile (1.05 g, 3.06 mmol) in anhydrous tetrahydrofuran (20 mL) at −40° C. was added Diisobutylaluminium hydride (6.1 mL, 6.10 mmol) under nitrogen atmosphere. The resulting mixture was stirred at room temperature for 2 hours. The reaction mixture was diluted with ice-cooled methanol, the mixture was filtered and concentrated under reduced pressure to give a crude residue which was purified by column chromatography using a 5% ethyl acetate in hexane gradient to afford 2′-methyl-6′-(4-(trifluoromethyl)cyclohexyl)-[1,1′-biphenyl]-2-carbaldehyde (0.570 g, 54%) as a colorless oil. ¹HNMR (400 MHz, CDCl₃): δ 9.66 (d, J=0.8 Hz, 1H), 8.05 (dd, J=8.0 Hz & 1.2 Hz, 1H), 7.69-7.65 (m, 1H), 7.53 (t, J=7.6 Hz, 1H), 7.32 (t, J=7.6 Hz, 1H), 7.22 (t, J=7.2 Hz, 2H), 7.14 (d, J=7.2 Hz, 1H), 2.21-1.96 (m, 5H), 1.93 (s, 3H), 1.77-1.63 (m, 2H), 1.52-1.31 (m, 3H).

2′-Methyl-6′-(4-(trifluoromethyl)cyclohexyl)-[1,1′-biphenyl]-2-carboxylic acid: To a mixture of 2′-methyl-6′-(4-(trifluoromethyl)cyclohexyl)-[1,1′-biphenyl]-2-carbaldehyde (0.570 g, 1.65 mmol) and 2-methylbut-2-ene (1.96 g, 28.00 mmol) in tetrahydrofuran-water (12 mL, 3 mL) at 0-5° C. was added sodium chlorite (447.0 mg, 4.94 mmol) and sodium phosphate monobasic (1.29 g, 8.25 mmol). The mixture was stirred at room temperature for 18 hours. The reaction mixture was extracted with ethyl acetate, washed with water and brine, dried over anhydrous sodium sulfate, and concentrated under reduced pressure to give a crude 2′-methyl-6′-(4-(trifluoromethyl)cyclohexyl)-[1,1′-biphenyl]-2-carboxylic acid (0.700 g, 94%) as a white solid.

Tert-butyl 2′-methyl-6′-(4-(trifluoromethyl)cyclohexyl)-[1,1′-biphenyl]-2-carboxylate: To a mixture of 2′-methyl-6′-(4-(trifluoromethyl)cyclohexyl)-[1,1′-biphenyl]-2-carboxylic acid (0.700 g, 1.54 mmol) in dichloromethane (15 mL) was added 2-tert-Butyl-1,3-diisopropyl-isourea (0.924 g, 4.62 mmol) at room temperature. After stirring at room temperature for 1 hour, more 2-tert-Butyl-1,3-diisopropyl-isourea (0.462 g, 2.31 mmol) was added at room temperature. The resulting mixture was stirred at room temperature under nitrogen atmosphere for 18 hours. The mixture was concentrated under reduced pressure to give a crude residue which was purified by column chromatography using a 2% ethyl acetate in hexane gradient to afford tert-butyl 2′-methyl-6′-(4-(trifluoromethyl)cyclohexyl)-[1,1′-biphenyl]-2-carboxylate (0.550 g, 85%) as a colorless oil. ¹HNMR (400 MHz, CDCl₃): δ 8.05 (dd, J=8.0 Hz & 1.2 Hz, 1H), 7.53-7.49 (m, 1H), 7.44-7.40 (m, 1H), 7.22 (t, J=7.6 Hz, 1H), 7.15 (d, J=7.6 Hz, 1H), 7.10-7.05 (m, 2H), 2.21-2.08 (m, 2H), 2.01-1.97 (m, 2H), 1.93 (s, 3H), 1.73-1.60 (m, 2H), 1.34-1.22 (m, 4H), 1.15 (s, 9H).

Tert-butyl 2′-(bromomethyl)-6′-(4-(trifluoromethyl)cyclohexyl)-[1,1′-biphenyl]-2-carboxylate: A mixture of tert-butyl 2′-methyl-6′-(4-(trifluoromethyl)cyclohexyl)-[1,1′-biphenyl]-2-carboxylate (0.260 g, 0.62 mmol), N-Bromosuccinimide (0.111 g, 0.62 mmol), and 2,2′-Azobis(2-methylpropionitrile) (0.020 g, 0.12 mmol) in CCl₄ (8 mL) was stirred at 95° C. under nitrogen atmosphere for 4 hours. The mixture was concentrated under reduced pressure to give a crude residue which was purified by column chromatography using a 2% ethyl acetate in hexane gradient to afford tert-butyl 2′-(bromomethyl)-6′-(4-(trifluoromethyl)cyclohexyl)-[1,1′-biphenyl]-2-carboxylate (0.120 g, 38%) as a colorless oil. TINMR (400 MHz, CDCl₃): δ 7.98 (d, J=7.6 Hz, 1H), 7.57-7.47 (m, 2H), 7.35-7.33 (m, 2H), 7.28-7.23 (m, 2H), 2.21-2.08 (m, 2H), 4.29 (d, J=10.0 Hz, 1H), 4.07 (d, J=10.0 Hz, 1H), 2.23-2.17 (m, 3H), 1.77-1.66 (m, 1H), 1.47-1.22 (m, 5H), 1.17 (s, 9H).

Tert-butyl 2′-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylate: To a mixture of sodium hydride (60% in mineral oil) (0.019 g, 0.48 mmol) in anhydrous tetrahydrofuran (4 mL) at 0-5° C. was added (1-(4-fluorobenzyl)-1H-pyrazol-4-yl)(8-(hydroxymethyl)-2-(1-(trifluoromethyl)cyclopropanecarbonyl)-2,6-diazaspiro[3.4]octan-6-yl)methanone prepared as described above (0.116 g, 0.24 mmol) in 2 mL of anhydrous tetrahydrofuran under nitrogen atmosphere. The resulting mixture was stirred at room temperature for 1 hour, followed by addition of tert-butyl 2′-(bromomethyl)-6′-(4-(trifluoromethyl)cyclohexyl)-[1,1′-biphenyl]-2-carboxylate (0.120 g, 0.24 mmol) in 2 mL of anhydrous tetrahydrofuran at 0-5° C. under nitrogen atmosphere. The resulting mixture was stirred at 40° C. for 15 hours. The mixture was poured into ice water (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate, and concentrated to give a crude residue which was purified by column chromatography using a 2% methanol in dichloromethane gradient to afford tert-butyl 2′-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylate (0.040 g, 19%) as a white solid. MS: [MH]⁺ 897.45.

2′-(((6-(1-(4-Fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylic acid: A mixture of tert-butyl 2′-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylate (0.040 g, 0.045 mmol) in 2,2,2-trifluoroacetic acid/dichloromethane (3 mL, 1:2) was stirred at 40° C. for 1 hour. The reaction mixture was concentrated under reduced pressure to give a crude residue which was purified by column chromatography using a 9% methanol in dichloromethane gradient to afford 2′-(((6-(1-(4-fluorobenzyl)-1H-pyrazole-4-carbonyl)-2-(1-(trifluoromethyl)cyclopropanecarbonyl)-2,6-diazaspiro[3.4]octan-8-yl)methoxy)methyl)-6′-(4-(trifluoromethyl)cyclohexyl)-[1,1′-biphenyl]-2-carboxylic acid (0.028 g, 74%) as a white solid. ¹HNMR (400 MHz, CD₃OD): δ 8.19-8.15 (m, 1H), 8.00-7.95 (m, 1H), 7.90-7.86 (m, 1H), 7.60-7.43 (m, 2H), 7.34-7.31 (m, 2H), 7.26-7.19 (m, 3H), 7.13-7.06 (m, 3H), 5.38-5.34 (m, 2H), 4.57 (s, 4H), 4.07-3.31 (m, 9H), 2.57-2.45 (m, 1H), 2.29-2.16 (m, 2H), 2.00-1.91 (m, 2H), 1.73-1.55 (m, 3H), 1.50-1.40 (m, 2H), 1.18-1.12 (m, 4H). MS: [MH]⁺ 841.40.

### Synthesis of (S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid I-127

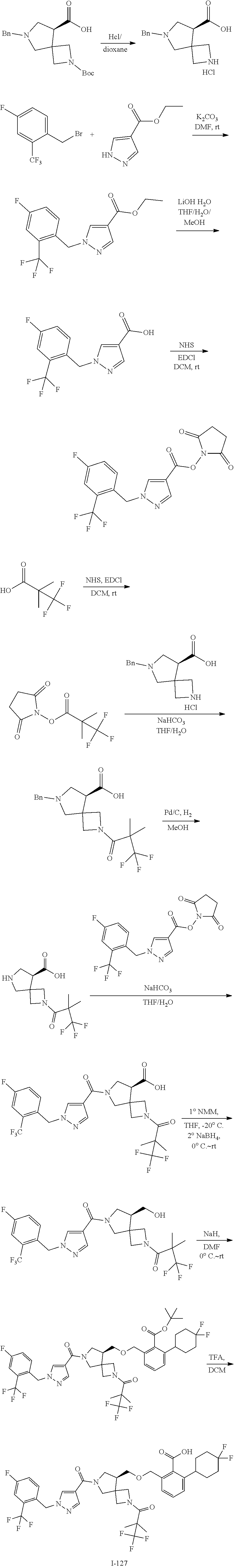

(S)-6-benzyl-2,6-diazaspiro[3.4]octane-8-carboxylic acid hydrochloride: To a solution of (S)-6-benzyl-2-(tert-butoxycarbonyl)-2,6-diazaspiro[3.4]octane-8-carboxylic acid (10.000 g, 28.90 mmol) in dichloromethane (40 mL) was added hydrogen chloride in dioxane (4 M, 40 mL), and the mixture was stirred at room temperature overnight. TLC showed the reaction was complete. The volatiles were evaporated under reduced pressure to afford (S)-6-benzyl-2,6-diazaspiro[3.4]octane-8-carboxylic acid hydrochloride (11.000 g, crude) as a white solid. ¹HNMR (400 MHz, CD₃OD): δ 7.65-7.62 (m, 2H), 7.50-7.48 (m, 3H), 4.53-4.50 (m, 2H), 4.39-4.24 (m, 3H), 4.12-3.98 (m, 1H), 3.87-3.69 (m, 3H), 3.64-3.54 (m, 2H). MS: [MH]⁺ 247.00.

Ethyl 1-(4-fluoro-2-(trifluoromethyl)benzyl)-1H-pyrazole-4-carboxylate: A mixture of 1-(bromomethyl)-4-fluoro-2-(trifluoromethyl)benzene (2.000 g, 7.80 mmol), ethyl 1H-pyrazole-4-carboxylate (0.908 g, 6.500 mmol), and potassium carbonate (1.300 g, 9.70 mmol) in N,N-dimethylformamide (20 mL) was stirred at room temperature under nitrogen atmosphere overnight. The reaction mixture was poured into water (20 mL) and extracted with ethyl acetate (40 mL). The organic layer was washed with water (20 mL×2) and brine (25 mL), dried over anhydrous sodium sulfate, and concentrated under reduced pressure to give a crude residue which was purified by column chromatography using a 6%˜20% ethyl acetate in hexane gradient to afford ethyl 1-(4-fluoro-2-(trifluoromethyl)benzyl)-1H-pyrazole-4-carboxylate (1.800 g, 90% yield) as a white solid. MS: [M+41]⁺ 358.50.

1-(4-Fluoro-2-(trifluoromethyl)benzyl)-1H-pyrazole-4-carboxylic acid: A mixture of ethyl 1-(4-fluoro-2-(trifluoromethyl)benzyl)-1H-pyrazole-4-carboxylate (1.800 g, 5.70 mmol) and lithium hydroxide monohydrate (0.957 g, 22.80 mmol) in tetrahydrofuran (8 mL)—water (2 mL)—methanol (2 mL) was stirred at 40° C. for 3 hours. The mixture solution was cooled to room temperature, acidified with hydrochloride acid (1 N) till pH 5-6, and extracted with ethyl acetate (15 mL×2). The combined organic layers were washed with brine (20 mL), dried over sodium sulfate, and concentrated under reduced pressure to give a crude residue which was purified by column chromatography using 33% ethyl acetate in hexane gradient to afford to afford 1-(4-fluoro-2-(trifluoromethyl)benzyl)-1H-pyrazole-4-carboxylic acid (1.300 g, 81% yield) as white solid. MS: [MH]⁺ 289.05.

2,5-Dioxopyrrolidin-1-yl 1-(4-fluoro-2-(trifluoromethyl)benzyl)-1H-pyrazole-4-carboxylate: To a solution of 1-(4-fluoro-2-(trifluoromethyl)benzyl)-1H-pyrazole-4-carboxylic acid (1.300 g, 4.50 mmol) in dichloromethane (20 mL) at room temperature was added 1-hydroxypyrrolidine-2,5-dione (0.623 g, 5.40 mmol) and 1-Ethyl-(3-dimethylaminopropyl)carbodiimide hydrochloride (1.100 g, 5.90 mmol). The mixture was stirred at room temperature for 2 hours, and then concentrated under reduced pressure to give a crude residue which was purified by column chromatography by using a 33% ethyl acetate in hexane gradient to afford 2,5-dioxopyrrolidin-1-yl 1-(4-fluoro-2-(trifluoromethyl)benzyl)-1H-pyrazole-4-carboxylate (1.500 g, 88% yield) as colorless oil. ¹HNMR (400 MHz, CDCl₃): δ 8.11 (s, 1H), 8.07 (s, 1H), 7.47-7.40 (m, 1H), 7.26-7.20 (m, 2H), 5.52 (s, 2H), 2.88 (s, 4H). [M+41]⁺ 427.10.

2,5-Dioxopyrrolidin-1-yl 3,3,3-trifluoro-2,2-dimethylpropanoate: To a solution of 3,3,3-trifluoro-2,2-dimethylpropanoic acid (0.500 g, 3.2 mmol) in dichloromethane (8 mL) at room temperature was added 1-hydroxypyrrolidine-2,5-dione (0.442 g, 3.80 mmol) and 1-Ethyl-(3-dimethylaminopropyl)carbodiimide hydrochloride (0.800 g, 4.20 mmol). The mixture was stirred at room temperature for 2 hours, and concentrated under reduced pressure to give a crude residue which was purified by column chromatography using a 25% ethyl acetate in hexane gradient to afford 2,5-dioxopyrrolidin-1-yl 3,3,3-trifluoro-2,2-dimethylpropanoate (0.670 g, 83% yield) as a white solid. ¹HNMR (400 MHz, CDCl₃): δ 1.90 (s, 6H).

(S)-6-benzyl-2-(3,3,3-trifluoro-2,2-dimethylpropanoyl)-2,6-diazaspiro[3.4]octane-8-carboxylic acid: To a solution of (S)-6-benzyl-2,6-diazaspiro[3.4]octane-8-carboxylic acid hydrochloride (0.500 g, 2.00 mmol) in tetrahydrofuran (8 mL)—water (8 mL) at room temperature was added sodium bicarbonate (0.831 g, 9.90 mmol), the mixture was stirred for 15 minutes, followed by the addition of 2,5-dioxopyrrolidin-1-yl 3,3,3-trifluoro-2,2-dimethylpropanoate (0.550 g, 2.20 mmol) in tetrahydrofuran (2 mL). The reaction mixture was stirred at room temperature overnight. TLC showed the reaction was completed. The mixture was acidified with hydrochloride acid (1 N) till pH 5-6, and concentrated under reduced pressure to give a crude residue which was purified by column chromatography by using a 10% methanol in dichloromethane gradient to afford (S)-6-benzyl-2-(3,3,3-trifluoro-2,2-dimethylpropanoyl)-2,6-diazaspiro[3.4]octane-8-carboxylic acid (0.600 g, 88% yield) as a white semi-solid. ¹HNMR (400 MHz, CDCl₃): δ 7.49-7.26 (m, 5H), 4.50-3.46 (m, 11H), 1.25 (s, 6H). MS: [MH]⁺ 385.70.

(S)-2-(3,3,3-trifluoro-2,2-dimethylpropanoyl)-2,6-diazaspiro[3.4]octane-8-carboxylic acid: A mixture of (S)-6-benzyl-2-(3,3,3-trifluoro-2,2-dimethylpropanoyl)-2,6-diazaspiro[3.4]octane-8-carboxylic acid (0.550 g, 1.40 mmol) and palladium on carbon (10%, 0.055 g) in methanol (15 mL) was stirred at room temperature under hydrogen atmosphere (hydrogen balloon) for 2 hours. Palladium on carbon was removed through filtration and washed with methanol (10 mL×2); The combined filtrates were concentrated under reduced pressure to afford crude (S)-2-(3,3,3-trifluoro-2,2-dimethylpropanoyl)-2,6-diazaspiro[3.4]octane-8-carboxylic acid (0.410 g, 100% yield) which was used in next step without further purification. MS: [MH]⁺ 294.90.

(S)-6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octane-8-carboxylic acid: To a solution of (S)-2-(3,3,3-trifluoro-2,2-dimethylpropanoyl)-2,6-diazaspiro[3.4]octane-8-carboxylic acid (0.400 g, 1.40 mmol) in tetrahydrofuran (6 mL)—water (6 mL) room temperature was added sodium bicarbonate (571.2 mg, 6.8 mmol); the mixture was stirred for 15 minutes, followed by the addition of 2,5-dioxopyrrolidin-1-yl 1-(4-fluoro-2-(trifluoromethyl)benzyl)-1H-pyrazole-4-carboxylate (0.790 g, 2.00 mmol), and the resulting mixture was stirred at room temperature overnight. TLC showed the reaction was completed. The reaction mixture was acidified with hydrochloride acid (1 N) till pH 5-6, and concentrated under reduced pressure to give a crude residue which was purified by column chromatography by using a 10% methanol in dichloromethane gradient to afford (S)-6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octane-8-carboxylic acid (0.220 g, 28% yield) as a white semi-solid. MS: [MH]⁺ 565.10.

(S)-3,3,3-trifluoro-1-(6-(1-(4-fluoro-2-(trifluoromethyl)benzyl)-1H-pyrazole-4-carbonyl)-8-(hydroxymethyl)-2,6-diazaspiro[3.4]octan-2-yl)-2,2-dimethylpropan-1-one: To a mixture of (S)-6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octane-8-carboxylic acid (0.190 g, 0.34 mmol) and 4-Methylmorpholine (0.069 g, 0.68 mmol) in anhydrous tetrahydrofuran (4 mL) at −20° C. under nitrogen atmosphere was added isobutyl carbonochloridate (138 mg, 1.01 mmol) in 2 mL of anhydrous tetrahydrofuran; the resulting mixture was stirred for 1 hour, followed by the addition of sodium borohydride (0.026 g, 0.68 mmol) at 0-5° C. After stirring at room temperature for 2 hours, the mixture was poured into ice water, and extracted with ethyl acetate (8 mL×3); The combined organic layers were washed with water and brine (15 mL each), dried over anhydrous sodium sulfate, and concentrated under reduced pressure to give a crude which was purified by prep-TLC using a 3% methanol in dichloromethane gradient to afford (S)-3,3,3-trifluoro-1-(6-(1-(4-fluoro-2-(trifluoromethyl)benzyl)-1H-pyrazole-4-carbonyl)-8-(hydroxymethyl)-2,6-diazaspiro[3.4]octan-2-yl)-2,2-dimethylpropan-1-one (0.048 g, 26%) as a white solid. MS: [MH]-551.15.

Tert-butyl (S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate: To a solution of (S)-3,3,3-trifluoro-1-(6-(1-(4-fluoro-2-(trifluoromethyl)benzyl)-1H-pyrazole-4-carbonyl)-8-(hydroxymethyl)-2,6-diazaspiro[3.4]octan-2-yl)-2,2-dimethylpropan-1-one (0.086 g, 0.16 mmol) in anhydrous tetrahydrofuran (2 mL) at 0-5° C. under nitrogen atmosphere was added sodium hydride (60% in mineral oil) (0.050 g, 1.25 mmol); the mixture was stirred for 0.5 hour, followed by the addition of tert-butyl 2-(bromomethyl)-6-(4,4-difluorocyclohexyl)benzoate (0.122 g, 0.31 mmol) in tetrahydrofuran (1 mL) at 0-5° C. under nitrogen atmosphere. The resulting mixture was stirred at room temperature for 3 hours. The reaction mixture was poured into water (10 mL) at 0° C., extracted with ethyl acetate (25 mL), washed with water (6 mL×3) and brine (15 mL), dried over anhydrous sodium sulfate, and concentrated under reduced pressure to give a crude residue which was purified by prep-TLC using a 50% ethyl acetate in dichloromethane gradient to afford tert-butyl (S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (0.043 g, 32%) as a white solid. MS: [MH]⁺ 859.45.

(S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid: A mixture of tert-butyl (S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (0.043 g, 0.053 mmol) in 2,2,2-trifluoroacetic acid/dichloromethane (3 mL, 1:1 V/V) was stirred at room temperature for 2 hours. The reaction mixture was concentrated under reduced pressure to give a crude residue which was purified by prep-HPLC to afford (S)-2-(4,4-difluorocyclohexyl)-6-(((6-(1-(4-fluoro-2-(trifluoromethyl)benzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (0.030 g, 63%) as an off-white solid. ¹HNMR (400 MHz, CD₃OD): δ 8.30-8.16 (m, 1H), 7.95 (d, J=13.6 Hz, 1H), 7.57-7.51 (m, 1H), 7.39-7.28 (m, 3H), 7.28-7.20 (m, 1H), 7.16-7.07 (m, 1H), 5.63-5.56 (m, 2H), 4.74-4.21 (m, 4H), 4.11-3.47 (m, 7H), 2.90-2.78 (m, 1H), 2.71-2.55 (m, 1H), 2.20-2.06 (m, 2H), 1.93-1.72 (m, 6H), 1.38-1.27 (m, 6H). MS: [MH]⁺ 803.35.

### Synthesis of 2-((((8S)-6-(1-(4-fluorobenzyl)-1H-pyrazole-4-carbonyl)-2-(2-methyloxetane-2-carbonyl)-2,6-diazaspiro[3.4]octan-8-yl)methoxy)methyl)-6-(4-(trifluoromethyl)cyclohexyl)benzoic acid I-93

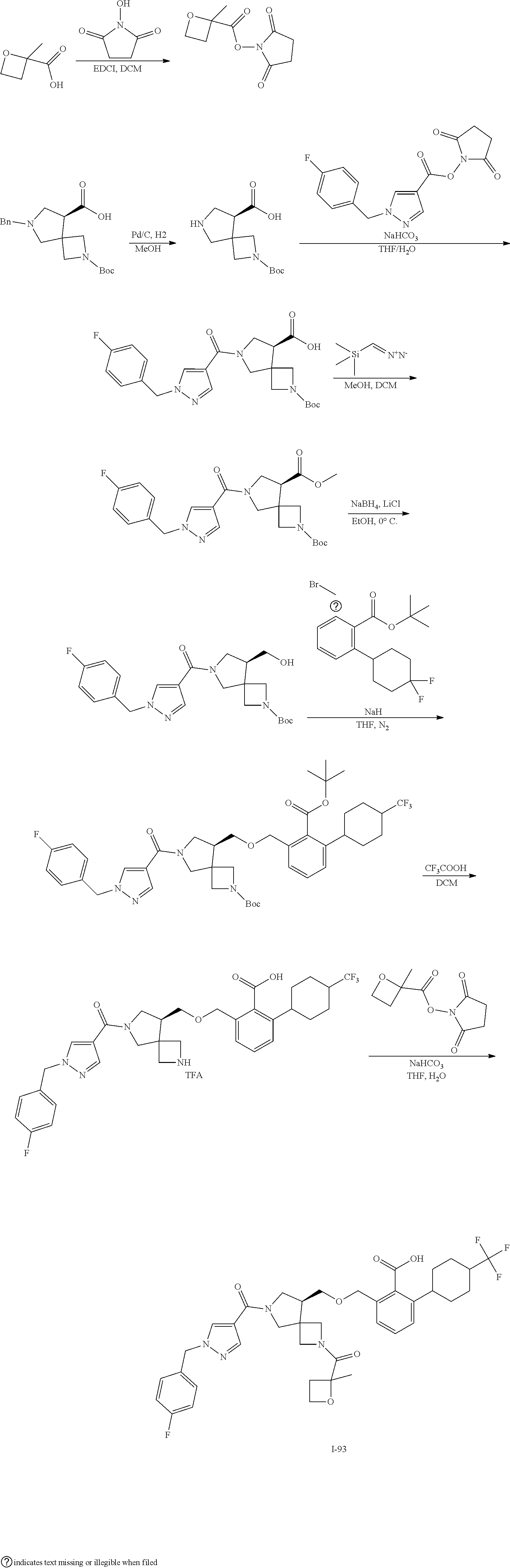

2,5-Dioxopyrrolidin-1-yl 2-methyloxetane-2-carboxylate: A mixture of 2-methyloxetane-2-carboxylic acid (0.100 g, 0.64 mmol), 1-hydroxypyrrolidine-2,5-dione (0.074 g, 0.64 mmol) and EDCI (0.147 g, 0.77 mmol) in dichloromethane (2 mL) was stirred at room temperature. The mixture was poured into water (5 mL) and extracted with dichloromethane (5 mL×2). The organic layers were collected and washed brine (10 mL), dried over anhydrous sodium sulfate, and concentrated under reduced pressure to afford 2,5-dioxopyrrolidin-1-yl 2-methyloxetane-2-carboxylate (0.154 g, 95%). ¹HNMR (400 MHz, CDCl₃): δ 4.78-4.68 (m, 2H), 3.10-3.05 (m, 2H), 2.85 (s, 4H), 1.35 (s, 3H), 1.62 (s, 6H).

(S)-2-(tert-butoxycarbonyl)-2,6-diazaspiro[3.4]octane-8-carboxylic acid: A mixture of (S)-6-benzyl-2-(tert-butoxycarbonyl)-2,6-diazaspiro[3.4]octane-8-carboxylic acid (2.000 g, 5.77 mmol) and Pd/C (0.400 g, 10%) in methanol (40 mL)—water (20 mL) was stirred at room temperature under hydrogen atomsphere overnight. The mixture was filtered and the Pd/C was washed with methanol (15 mL×3), the combined filtrates was concentrated to afford (S)-2-(tert-butoxycarbonyl)-2,6-diazaspiro[3.4]octane-8-carboxylic acid (1.250 g, 84%) as an off-white solid. ¹HNMR (400 MHz, DMSO-d6):9.78 (br, 1H), 3.83-3.92 (m, 4H), 3.31-3.42 (m, 5H), 1.38 (s, 9H).

(S)-2-(tert-butoxycarbonyl)-6-(1-(4-fluorobenzyl)-1H-pyrazole-4-carbonyl)-2,6-diazaspiro[3.4]octane-8-carboxylic acid: To a solution of (S)-2-(tert-butoxycarbonyl)-2,6-diazaspiro[3.4]octane-8-carboxylic acid (1.250 g, 4.88 mmol) in water (20 mL) at 0° C. was added NaHCO₃ (1.23 g, 14.64 mmol), the mixture was stirred for 10 min, followed by the addition of 2,5-dioxopyrrolidin-1-yl 1-(4-fluorobenzyl)-1H-pyrazole-4-carboxylate (1.550 g, 4.88 mmol) in THE (10 mL) slowly at 0° C. The resulting mixture was stirred at room temperature for 1 hour. TLC showed the reaction was complete. The reaction mixture was then poured into water (15 ml) and extracted with ethyl acetate (20 ml×2). The aqueous layer was adjusted pH 3-4 with hydrochloric acid (2.0N), extracted with 10% methanol in dichloromethane (20 mL×5). The combined organic layers were dried over anhydrous sodium sulfate, and concentrated under reduced pressure to give a residue which purified by column chromatography by using a 5-10% methanol in dichloromethane gradient to afford (S)-2-(tert-butoxycarbonyl)-6-(1-(4-fluorobenzyl)-1H-pyrazole-4-carbonyl)-2,6-diazaspiro[3.4]octane-8-carboxylic acid (1.810 g, yield 81%) as a white solid. MS: [MH]⁺ 459.1

2-(Tert-butyl) 8-methyl (S)-6-(1-(4-fluorobenzyl)-1H-pyrazole-4-carbonyl)-2,6-diazaspiro[3.4]octane-2,8-dicarboxylate: To a solution of (S)-2-(tert-butoxycarbonyl)-6-(1-(4-fluorobenzyl)-1H-pyrazole-4-carbonyl)-2, 6-diazaspiro[3.4]octane-8-carboxylic acid (1.036 g, 2.26 mmol) in methanol (12 mL)—dichloromethane (50 mL) at 0° C. was added (diazomethyl)trimethylsilane (2 M in hexane, 6.0 mL). The mixture was stirred at room temperature for 12 hours. The reaction mixture was treated with water (50 mL), extracted with ethyl acetate (20 mL*3), dried with sodium sulfate, and concentrated by reduced pressure to give a residue which was purified through column chromatography using a 3% methanol in dichloromethane gradient to afford (S)-2-tert-butyl 8-methyl 6-(1-(4-fluorobenzyl)-1H-pyrazole-4-carbonyl)-2,6-diazaspiro[3.4]octane-2,8-dicarboxylate (0.992 g, 93% yield) as a yellow oil. MS: [MH]⁺ 473.2

(S)-tert-butyl 6-(1-(4-fluorobenzyl)-1H-pyrazole-4-carbonyl)-8-(hydroxymethyl)-2,6-diazaspiro[3.4]octane-2-carboxylate: To a solution of (S)-2-tert-butyl 8-methyl 6-(1-(4-fluorobenzyl)-1H-pyrazole-4-carbonyl)-2,6-diazaspiro[3.4]octane-2,8-dicarboxylate (0.796 g, 0.84 mmol) in EtOH (10 mL) at 0° C. was added LiCl (0.361 g, 8.44 mmol) and NaBH₄ (0.319 g, 8.44 mmol), and the resulting mixture was stirred at 0° C. for 16 hours. TLC showed the reaction was complete. The reaction mixture was poured into saturated aqueous ammonium chloride solution (30 mL), and extracted with ethyl acetate (25 mL×2). The combined organic layers were washed with brine (25 mL), dried with sodium sulfate, and concentrated under reduced pressure to give a residue which was purified through column chromatography by using a 3-5% methanol in dichloromethane gradient to afford (S)-tert-butyl 6-(1-(4-fluorobenzyl)-1H-pyrazole-4-carbonyl)-8-(hydroxymethyl)-2,6-diazaspiro[3.4]octane-2-carboxylate (0.572 g, 76% yield) as a yellow oil. MS: [MH]⁺ 445.1.

(S)-tert-butyl 8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate: To a solution of (S)-tert-butyl 6-(1-(4-fluorobenzyl)-1H-pyrazole-4-carbonyl)-8-(hydroxymethyl)-2,6-diazaspiro[3.4]octane-2-carboxylate (0.572 g, 1.22 mmol) in anhydrous DMF (20 mL) was added NaH (60% in material oil, 0.121 g, 3.02 mmol) at 0° C., and the resulting mixture was stirred for 30 min, followed by the addition of tert-butyl 2-(bromomethyl)-6-(4, 4-difluorocyclohexyl)benzoate (2.816 g, 7.24 mmol) in anhydrous DMF (5 mL) slowly at 0° C. under nitrogen. The resulting mixture was stirred at room temperature under nitrogen for 5 hours. TLC showed the reaction was complete. The reaction mixture was poured into water (25 mL), extracted with ethyl acetate (20 mL×3). The combined organic layers were washed with saturated aqueous ammonium chloride solution (15 mL×2), dried with sodium sulfate, and concentrated under reduced pressure to give a residue which was purified through column chromatography using a 3% methanol in dichloromethane gradient to afford (S)-tert-butyl-8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate (0.804 g, 79% yield) as a yellow solid. MS: [MH]⁺ 785.50.

(S)-2-(((6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid TFA salt: A mixture of (S)-tert-butyl-8-(((2-(tert-butoxycarbonyl)-3-(4,4-difluorocyclohexyl)benzyl)oxy)methyl)-6-(1-(4-fluorobenzyl)-1H-pyrazole-4-carbonyl)-2,6-diazaspiro[3.4]octane-2-carboxylate (0.804 g, 1.02 mmol) and TFA (8 mL) in dichloromethane (10 mL) was stirred at room temperature for 3 h. The volatiles were removed under reduced pressure to give a crude residue which was triturated with ether (15 mL). The resulting solid was collected by filtration and dried in vacuum to afford (S)-2-(((6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid TFA salt (0.628 g, 85% yield) as a yellow solid. MS: [MH]⁺ 629.3.

2-((((8S)-6-(1-(4-fluorobenzyl)-1H-pyrazole-4-carbonyl)-2-(2-methyloxetane-2-carbonyl)-2,6-diazaspiro[3.4]octan-8-yl)methoxy)methyl)-6-(4-(trifluoromethyl)cyclohexyl)benzoic acid: To a stirred mixture of afford (S)-2-(((6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid TFA salt (0.100 g, 0.14 mmol) in THF (2 mL) at 0° C. was added NaHCO₃ (0.118 g, 1.40 mmol, in 2 mL of H₂O), the resulting mixture was stirred for 20 min, followed by the addition of added 2,5-dioxopyrrolidin-1-yl 2-methyloxetane-2-carboxylate prepared as described above (0.089 g, 0.42 mmol), the reaction mixture was stirred at room temperature overnight. TLC showed the reaction was completed. The reaction mixture was acidified with aqueous hydrochloric acid (2 M) to pH 4-5, extracted with ethyl acetate (10 mL*3), dried over sodium sulfate, and concentrated to give a residue which was purified through Prep-TLC using 10% dichloromethane in methanol gradient to afford 2-((((8S)-6-(1-(4-fluorobenzyl)-1H-pyrazole-4-carbonyl)-2-(2-methyloxetane-2-carbonyl)-2,6-diazaspiro[3.4]octan-8-yl)methoxy)methyl)-6-(4-(trifluoromethyl)cyclohexyl)benzoic acid (0.060 g, yield 59%) as a white solid. ¹H NMR (400 MHz, CDCl₃) δ 8.29-8.20 (m, 1H), 7.92-7.90 (m, 1H), 7.34-7.20 (m, 5H), 7.09-7.04 (m, 2H), 5.35 (s, 2H), 5.35-3.56 (m, 14H), 2.85-2.41 (m, 5H), 2.08-1.90 (m, 3H), 1.74 (s, 3H), 1.58-1.52 (m, 3H), 1.44-1.34 (m, 2H). MS: [MH]⁺ 727.4.

The following compounds were prepared in a manner analogous to the procedures described above for 2-((((8S)-6-(1-(4-fluorobenzyl)-1H-pyrazole-4-carbonyl)-2-(2-methyloxetane-2-carbonyl)-2,6-diazaspiro[3.4]octan-8-yl)methoxy)methyl)-6-(4-(trifluoromethyl)cyclohexyl)benzoic acid I-93.

(S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4-(trifluoromethyl)cyclohexyl)benzoic acid I-94 (0.045 g, yield 18%) as white solid: ¹H NMR (400 MHz, CD₃OD) δ 8.28-8.19 (m, 1H), 7.92-7.90 (m, 1H), 7.34-7.12 (m, 5H), 7.10-7.04 (m, 2H), 5.35 (s, 2H), 4.70-4.52 (m, 3H), 4.40-4.22 (m, 1H), 4.06-3.88 (m, 3H), 3.76-3.52 (m, 4H), 2.84-2.62 (m, 2H), 2.42-2.17 (m, 2H), 2.07-1.93 (m, 3H), 1.74 (s, 3H), 1.59-1.53 (m, 1H), 1.39 (s, 3H), 1.37-1.33 (m, 4H). MS: [MH]⁺ 767.4

(S)-2-(((6-(1-(4-fluorobenzyl)-1H-pyrazole-4-carbonyl)-2-(1-(trifluoromethyl)cyclobutanecarbonyl)-2,6-diazaspiro[3.4]octan-8-yl)methoxy)methyl)-6-(4-(trifluoromethyl)cyclohexyl)benzoic acid I-95 (0.045 g, yield 24%) as white solid: ¹H NMR (400 MHz, CD₃OD) δ 8.29-8.20 (m, 1H), 7.92-7.90 (m, 1H), 7.34-7.19 (m, 5H), 7.09-7.05 (m, 2H), 5.36 (s, 2H), 4.66-3.62 (m, 12H), 2.84-1.95 (m, 11H), 1.74 (s, 4H), 1.63-1.34 (m, 1H). MS: [MH]⁺ 779.4.

2-((((8S)-6-(1-(4-fluorobenzyl)-1H-pyrazole-4-carbonyl)-2-(3,3,3-trifluoro-2-methylpropanoyl)-2,6-diazaspiro[3.4]octan-8-yl)methoxy)methyl)-6-(4-(trifluoromethyl)cyclohexyl)benzoic acid I-97 (0.029 g, yield 18%) as white solid: ¹H NMR (400 MHz, CD₃OD) δ 8.30-8.18 (m, 1H), 7.93-7.89 (m, 1H), 7.34-7.06 (m, 7H), 5.35 (s, 2H), 4.62-4.50 (m, 2H), 4.39-3.46 (m, 10H), 2.83-2.61 (m, 2H), 2.46-2.37 (m, 1H), 2.19-1.94 (m, 3H), 1.74-1.54 (m, 4H), 1.32-1.15 (m, 5H). MS: [MH]⁺ 753.50

(S)-2-(((6-(1-(4-fluorobenzyl)-1H-pyrazole-4-carbonyl)-2-(3,3,3-trifluoro-2-methyl-2-(trifluoromethyl)propanoyl)-2,6-diazaspiro[3.4]octan-8-yl)methoxy)methyl)-6-(4-(trifluoromethyl)cyclohexyl)benzoic acid I-100 (0.018 g, yield 14%) as white solid: ¹H NMR (400 MHz, CD₃OD) δ 8.19-8.29 (m, 1H), 7.92-7.89 (m, 1H), 7.31-7.17 (m, 5H), 7.09-7.04 (m, 2H), 5.38 (s, 2H), 4.60 (s, 2H), 3.45-4.45 (m, 9H), 2.85-2.75 (m, 1H), 2.72-2.53 (m, 1H), 2.48-2.15 (m, 3H), 2.10-1.90 (m, 3H), 1.79-1.49 (m, 5H), 1.38-1.25 (m, 3H).

### Synthesis of (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluoropiperidin-1-yl)benzoic acid I′-10

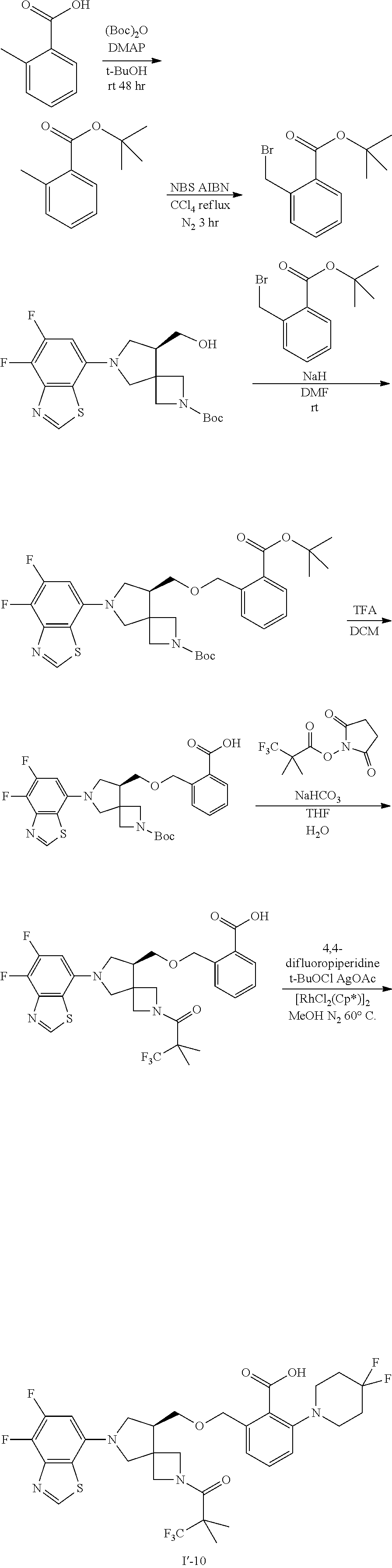

Tert-butyl 2-methylbenzoate: To a stirred solution of 2-methylbenzoic acid (5.0 g, 0.04 mol) and (Boc)₂O (16.0 g, 0.07 mmol) in t-BuOH (100 mL) at 0° C. was added DMAP (2.24 g, 0.02 mol) in portions. The mixture was stirred at room temperature for 48 hours. The reaction mixture was concentrated under reduced pressure to give a residue which was purified by column chromatography using a 100% hexane gradient to afford tert-butyl 2-methylbenzoate (2.95 g, 42%) as a colorless oil. ¹HNMR (400 MHz, CDCl₃): δ 7.83-7.81 (m, 1H), 7.37-7.33 (m, 1H), 7.24-7.20 (m, 2H), 2.57 (s, 3H), 1.60 (s, 9H).

Tert-butyl 2-(bromomethyl)benzoate: A solution of tert-butyl 2-methylbenzoate (0.370 g, 1.93 mmol), NBS (0.377 g, 2.12 mmol), and AIBN (0.063 g, 0.39 mmol) in CCl₄ (10 mL) was stirred at 90° C. under N₂ atmosphere for 8 hours. After cooling, the reaction mixture was concentrated under reduced pressure to give a residue which was purified by column chromatography using a 1% ethyl acetate in hexane gradient to afford tert-butyl 2-(bromomethyl)benzoate (0.360 g, 69%) as a red oil. ¹HNMR (400 MHz, CDCl₃): δ 7.89-7.87 (m, 1H), 7.47-7.40 (m, 2H), 7.37-7.33 (m, 1H), 4.92 (s, 2H), 1.64 (s, 9H).

Tert-butyl (S)-8-(((2-(tert-butoxycarbonyl)benzyl)oxy)methyl)-6-(4,5-difluorobenzo[d]thiazol-7-yl)-2,6-diazaspiro[3.4]octane-2-carboxylate: To a stirred solution of tert-butyl (S)-6-(4,5-difluorobenzo[d]thiazol-7-yl)-8-(hydroxymethyl)-2,6-diazaspiro[3.4]octane-2-carboxylate (0.150 g, 0.36 mol) in anhydrous THE (3 mL) at 0° C. was added NaH (0.073 g, 1.82 mmol, 60% in mineral oil); the mixture was stirred at room temperature under N₂ for 0.5 hours, followed by the addition of tert-butyl 2-(bromomethyl)benzoate (0.150 g, 0.55 mmol) in THF (2 mL) at room temperature, and the resulting mixture was stirred at 30° C. for 4 h. The reaction mixture was poured into water (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate, and concentrated to give residue which was purified by column chromatography using a 3-5% ethyl acetate in hexane gradient to afford tert-butyl (S)-8-(((2-(tert-butoxycarbonyl)benzyl)oxy)methyl)-6-(4,5-difluorobenzo[d]thiazol-7-yl)-2,6-diazaspiro[3.4]octane-2-carboxylate (0.120 g, 55%) as a red oil. MS: [MH]⁺ 602.20.

(S)-2-(((6-(4,5-difluorobenzo[d]thiazol-7-yl)-2,6-diazaspiro[3.4]octan-8-yl)methoxy)methyl)benzoic acid: A solution of tert-butyl (S)-8-(((2-(tert-butoxycarbonyl)benzyl)oxy)methyl)-6-(4,5-difluorobenzo[d]thiazol-7-yl)-2,6-diazaspiro[3.4]octane-2-carboxylate (0.175 g, 0.29 mmol) and TFA (2 mL) in DCM (2 mL) was stirred at room temperature for 4 hours. The mixture was concentrated in vacuo to give (S)-2-(((6-(4,5-difluorobenzo[d]thiazol-7-yl)-2,6-diazaspiro[3.4]octan-8-yl)methoxy)methyl)benzoic acid (170 mg, crude) as a red solid. MS: [MH]⁺ 446.95.

(S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)benzoic acid: To a stirred mixture of (S)-2-(((6-(4,5-difluorobenzo[d]thiazol-7-yl)-2,6-diazaspiro[3.4]octan-8-yl)methoxy)methyl)benzoic acid (0.170 g, 0.38 mmol) and NaHCO₃ (0.320 g, 3.80 mmol) in H₂O (5.0 mL) at RT was added 2,5-dioxopyrrolidin-1-yl 3,3,3-trifluoro-2,2-dimethylpropanoate (0.150 g, 0.57 mmol) in THE (5 mL), and the solution was stirred at RT overnight. TLC showed the reaction was completed. The reaction mixture was acidified with hydrochloric acid (2 M) to pH 4-5, extracted with ethyl acetate (10 mL*3), dried over sodium sulfate, and concentrated to give a residue which was purified through prep-TLC using 10% methanol in dichloromethane gradient to afford (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)benzoic acid (0.030 g, yield 14%) as red solid. MS: [MH]⁺ 647.25.

(S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluoropiperidin-1-yl)benzoic acid: To a mixture of (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)benzoic acid (0.030 g, 0.05 mmol), [RhCl2-(Cp*)]2 (0.001 g, 0.002 mmol), and AgOAc (0.013 g, 0.08 mmol) in MeOH (2.5 mL) under a N₂ atmosphere at room temperature was added a solution of 4,4-difluoropiperidine (0.029 g, 0.24 mmol)-tert-butyl hypochlorite (0.026 g, 0.24 mmol) in dioxane (1 mL) dropwise. The resulting mixture was stirred at 60° C. for 16 h till completion. The reaction mixture was poured into water (10 mL) and extracted with ethyl acetate (10 mL). The organic layer was collected, and the aqueous layer was extracted with ethyl acetate (10 mL×2). The combined organic layers were washed with brine (10 mL), dried over anhydrous sodium sulfate, and concentrated to give a crude residue which was purified by prep-HPLC to afford (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluoropiperidin-1-yl)benzoic acid (0.006 g, 17%) as a gray solid. ¹HNMR (400 MHz, CDCl₃): δ 9.15 (s, 1H), 7.80 (d, J=5.2 Hz, 1H), 7.53-7.49 (m, 1H), 7.34 (d, J=8.0 Hz), 6.42-6.38 (m, 1H), 5.05 (s, 2H), 4.46-3.92 (m, 4H), 3.82-3.72 (m, 5H), 3.51-3.48 (m, 1H), 3.18 (t, J=2.8 Hz, 4H), 2.75-2.73 (m, 1H). 2.34-2.24 (m, 4H), 1.40 (s, 6H). MS: [MH]⁺ 703.25.

### Synthesis of (S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoicacid I′-34

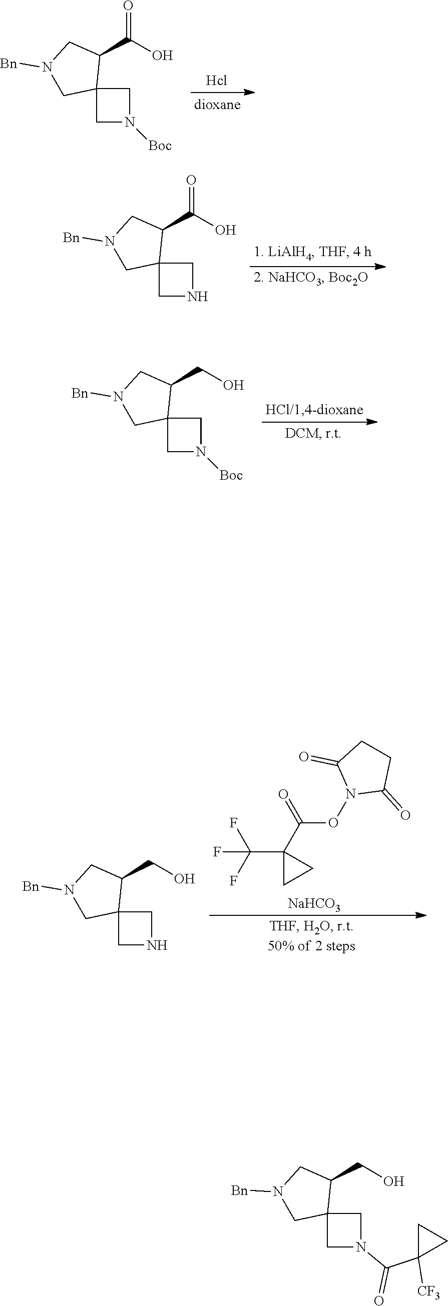

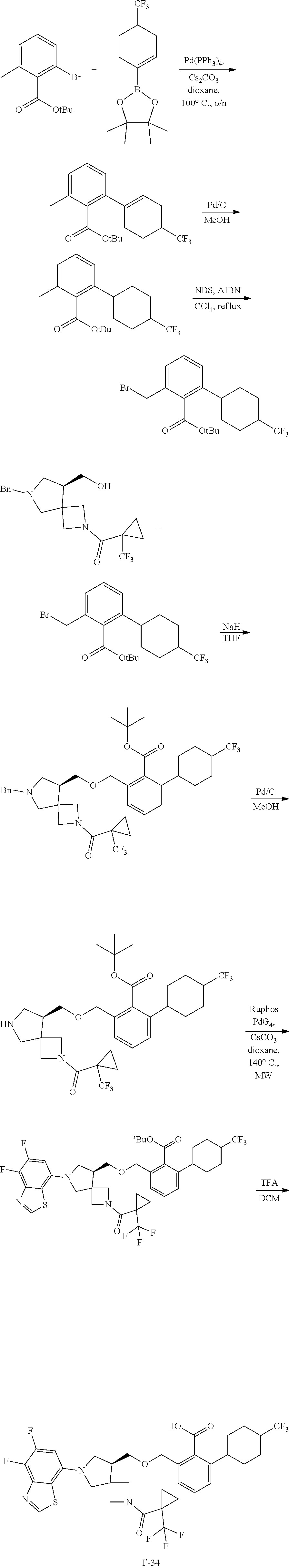

(S)-6-benzyl-2,6-diazaspiro[3.4]octane-8-carboxylic acid hydrochloride: To a solution of (S)-6-benzyl-2-(tert-butoxycarbonyl)-2,6-diazaspiro[3.4]octane-8-carboxylic acid (10.000 g, 28.90 mmol) in dichloromethane (40 mL) was added hydrogen chloride in dioxane (4 M, 40 mL), and the resulting mixture was stirred at room temperature overnight. TLC showed the reaction was complete. The volatiles were evaporated under reduced pressure to afford (S)-6-benzyl-2,6-diazaspiro[3.4]octane-8-carboxylic acid hydrochloride (11.000 g, crude) as a white solid. MS: [MH]⁺ 247.00. ¹HNMR (400 MHz, CD₃OD): δ 7.65-7.62 (m, 2H), 7.50-7.48 (m, 3H), 4.53-4.50 (m, 2H), 4.39-4.24 (m, 3H), 4.12-3.98 (m, 1H), 3.87-3.69 (m, 3H), 3.64-3.54 (m, 2H).

(S)-tert-butyl 6-benzyl-8-(hydroxymethyl)-2,6-diazaspiro[3.4]octane-2-carboxylate: To a solution of (S)-6-benzyl-2,6-diazaspiro[3.4]octane-8-carboxylic acid hydrochloride (11.000 g, crude) in anhydrous tetrahydrofuran (170 mL) was added LiAlH₄ (3.300 g, 86.59 mmol) in several batches to keep the inner reaction temperature at 0° C. After the addition was completed, the resulting mixture was warmed to room temperature, and stirred under nitrogen atmosphere for 3 hours. TLC showed the reaction was complete. The reaction mixture was cooled to 0° C. and quenched with water (3.3 mL)—aqueous sodium hydroxide solution (3.3 ml, 15% in water)—water (9.9 mL). The resulting mixture was stirred for 30 min, followed by addition of water (100 mL) and di-tert-butyl dicarbonate (12.600 g, 57.33 mmol). The mixture was stirred at room temperature overnight. TLC showed the reaction was complete. The reaction mixture was filtered, and the filtrate was extracted with ethyl acetate (50 mL×2). The combined organic layer was washed with brine (50 mL×2), dried over anhydrous sodium sulfate, and concentrated under reduced pressure to give a crude residue which was purified by column chromatography using 50-75% ethyl acetate in hexane gradient to afford (S)-tert-butyl 6-benzyl-8-(hydroxymethyl)-2,6-diazaspiro[3.4]octane-2-carboxylate (4.4 g, 46% three steps) as colorless oil. MS: [MH]⁺ 333.50. ¹HNMR (400 MHz, CDCl₃): δ 7.33-7.28 (m, 4H), 7.26-7.25 (m, 1H), 4.14 (d, J=8.8 Hz, 2H), 3.87-3.82 (m, 2H), 3.76 (d, J=4.4 Hz, 2H), 3.72 (d, J=9.2 Hz, 1H), 3.58 (s, 2H), 2.93 (d, J=10.0 Hz, 1H), 2.72-2.67 (m, 2H), 2.63-2.60 (m, 1H), 2.27-2.24 (m, 1H), 1.42 (s, 9H).

(S)-(6-benzyl-2,6-diazaspiro[3.4]octan-8-yl)-methanol: A mixture of (S)-tert-butyl 6-benzyl-8-(hydroxymethyl)-2,6-diazaspiro[3.4]octane-2-carboxylate (3.090 g, 9.31 mmol) and hydrogen chloride dioxane solution (4 M, 30 mL) in dichloromethane (30 mL) was stirred at room temperature overnight. TLC showed the reaction was complete. The volatiles were evaporated under reduced pressure to afford (S)-(6-benzyl-2,6-diazaspiro[3.4]octan-8-yl)-methanol (2.160 g, crude) as white solid which was used in next step without further purification. MS: [MH]⁺ 233.10.

(S)-(6-benzyl-8-(hydroxymethyl)-2,6-diazaspiro[3.4]octan-2-yl)(1-(trifluoromethyl)cyclopropyl)methanone: To a solution of (S)-(6-benzyl-2,6-diazaspiro[3.4]octan-8-yl)methanol (2.160 g, crude) in water (50 mL) was added sodium bicarbonate (3.900 g, 46.55 mmol), followed by addition of 2,5-dioxopyrrolidin-1-yl 1-(trifluoromethyl)cyclopropanecarboxylate (3.500 g, 13.97 mmol) in tetrahydrofuran (50 mL), and the resulting mixture was stirred at room temperature for 4 hours. TLC showed the reaction was complete. The reaction mixture was extracted with ethyl acetate (30 mL×3). The combined organic phases were washed with brine (30 mL×2), dried over anhydrous sodium sulfate, and concentrated under reduced pressure to give a crude residue which was purified by column chromatography using 1% methanol in dichloromethane gradient to afford (S)-(6-benzyl-8-(hydroxymethyl)-2,6-diazaspiro[3.4]octan-2-yl)(1-(trifluoromethyl)cyclopropyl)methanone (1.780 g, 50% yield over two steps) as colorless oil. ¹HNMR (400 MHz, CDCl₃): δ 7.34-7.26 (m, 5H), 4.54-4.20 (m, 2H), 3.91-4.06 (m, 2H), 3.77-3.75 (m, 2H), 3.62-3.58 (m, 2H), 2.98-2.68 (m, 1H), 2.77-2.69 (m, 2H), 2.63-2.53 (m, 1H), 1.18-1.17 (m, 4H). MS: [MH]⁺ 369.40.

Tert-butyl 3-methyl-4′-(trifluoromethyl)-2′,3′,4′,5′-tetrahydro-[1,1′-biphenyl]-2-carboxylate: To a stirred mixture of tert-butyl 2-bromo-6-methylbenzoate (0.560 g, 2.07 mmol), 4,4,5,5-tetramethyl-2-(4-(trifluoromethyl)cyclohex-1-en-1-yl)-1,3,2-dioxaborolane (0.570 g, 2.06 mmol), and cesium carbonate (1.400 g, 4.29 mmol) in dioxane (5 mL) under nitrogen atmosphere was added tetrakis(triphenylphosphine)palladium (0.238 g, 0.21 mmol), and the resulting mixture was stirred under N₂ at 100° C. overnight. TLC showed the reaction was complete. The reaction mixture was poured into water (10 mL) at room temperature and extrated with ethyl acetate (15 mL). The organic layer was collected, washed with water (15 mL), dried over anhydrous sodium sulfate, and concentrated under reduced pressure to give a crude residue which was purified by column chromatography by using a 5% ethyl acetate in hexane to afford tert-butyl 3-methyl-4′-(trifluoromethyl)-2′,3′,4′,5′-tetrahydro-[1,1′-biphenyl]-2-carboxylate (0.630 g, 89% yield) as a colorless oil. ¹HNMR (400 MHz, CDCl₃): δ 7.22 (t, J=15.2 Hz, 1H), 7.10 (t, J=17.6 Hz, 1H), 6.98 (d, J=7.6 Hz, 1H), 5.64 (s, 1H), 2.44-2.40 (m, 2H), 2.35 (s, 3H), 2.32 (s, 1H), 2.24-2.10 (m, 2H), 1.79-1.65 (m, 2H), 1.54 (s, 9H).

Tert-butyl 2-methyl-6-(4-(trifluoromethyl)cyclohexyl)benzoate: A mixture of tert-butyl 3-methyl-4′-(trifluoromethyl)-2′,3′,4′,5′-tetrahydro-[1,1′-biphenyl]-2-carboxylate (0.600 g, 1.76 mmol) and Pd/C (0.200 g) in methanol (50 mL) was stirred at room temperature under hydrogen atomsphere overnight. Palladium on carbon was removed through filtration and washed with methanol (15 mL×2). The combined filtrates were concentrated to afford tert-butyl 2-methyl-6-(4-(trifluoromethyl)cyclohexyl)benzoate (600 mg, 100%) as a colorless oil which was used in next step without further purification. ¹HNMR (400 MHz, CDCl₃): δ 7.22 (d, J=7.6 Hz, 1H), 7.11 (d, J=7.6 Hz, 1H), 7.03 (d, J=7.2 Hz, 1H), 2.70-2.62 (m, 1H), 2.42-2.35 (m, 1H), 2.32 (s, 3H), 2.14 (d, J=14 Hz, 2H), 2.06 (t, J=26.4 Hz, 1H), 1.79-1.72 (m, 2H), 1.70-1.64 (m, 2H), 1.61 (s, 9H), 1.51-1.43 (m, 1H).

Tert-butyl 2-(bromomethyl)-6-(4-(trifluoromethyl)cyclohexyl) benzoate: To a stirred solution of tert-butyl 2-methyl-6-(4-(trifluoromethyl)cyclohexyl)benzoate (0.850 g, 2.48 mmol) in CCl₄ (5 mL) was added 2,2′-Azobis(2-methylpropionitrile) (0.033 g, 0.20 mmol) and NBS (0.427 g, 2.40 mmol) at room temperature under N₂. The resulting mixture was stirred at 90° C. for 3 hours under N₂. TLC showed the reaction was complete. The mixture was concentrated under reduced pressure to give a crude residue which was purified by silica gel column chromatography using a 1% ethyl acetate in hexane to afford tert-butyl 2-(bromomethyl)-6-(4-(trifluoromethyl)cyclohexyl)benzoate (0.600 g, 60% yield) as colorless oil. ¹HNMR (400 MHz, CDCl₃): δ 7.34-7.31 (m, 1H), 7.25-7.21 (m, 1H), 7.12-6.97 (m, 1H), 4.53 (s, 2H), 2.76-2.65 (m, 1H), 2.43-2.32 (m, 1H), 2.16-2.03 (m, 4H), 1.81-1.73 (m, 4H), 1.65 (s, 9H).

Tert-butyl (S)-2-(((6-benzyl-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate: To a stirred solution of (S)-(6-benzyl-8-(hydroxymethyl)-2,6-diazaspiro[3.4]octan-2-yl)(1-(trifluoromethyl)cyclopropyl)methanone (0.050 g, 0.14 mmol) in anhydrous THF (2 mL) at 0° C. was added sodium hydride (60% in mineral oil) (100 mg, 0.84 mmol). The resulting mixture was stirred at room temperature for 1 hour, followed by the addition of tert-butyl 2-(bromomethyl)-6-(4-(trifluoromethyl)cyclohexyl)benzoate (0.057 g, 0.14 mmol), the resulting mixture was stirred at room temperature overnight. TLC showed the reaction was complete. The reaction mixture was poured into water (15 mL) and extracted with ethyl acetate (10 mL×2). The organic layer was collected, washed with water (10 mL×3) and brine (10 mL), dried over anhydrous sodium sulfate, and concentrated under reduced pressure to give a crude residue which was column chromatography using 5% methanol in dichloromethane gradient to afford tert-butyl (S)-2-(((6-benzyl-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (0.045 mg, 47% yeild) as a white solid. MS: [MH]⁺ 709.2.

Tert-butyl (S)-2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate: A mixture of tert-butyl (S)-2-(((6-benzyl-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (0.250 g, 0.35 mmol) and Pd/C (0.050 g) in methanol (50 mL) was stirred at room temperature under hydrogen atomsphere overnight. Palladium on carbon was removed through filtration and washed with methanol (15 mL×2). The combined filtrates were concentrated to afford tert-butyl (S)-2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate (0.220 g, 100%) as a colorless oil which was used in next step without further purification. MS: [MH]⁺ 619.3.

Tert-butyl (S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate: To a stirred solution of tert-butyl (S)-2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate (0.030 g, 0.05 mmol) and 7-bromo-4,5-difluorobenzo[d]thiazole (0.014 g, 0.06 mmol) in toluene (2 mL) was added RuphosPdG₄ (0.008 g, 0.01 mmol) and cesium carbonate (0.024 g, 0.07 mmol) at room temperature under N₂. The resulting mixture was stirred at 150° C. under microwave irradiation for 0.5 hour. TLC showed the reaction was complete. The reaction mixture was concentrated under reduced pressure to give a crude residue which was prep-TLC using a 50% ethyl acetate in hexane containing 5% methanol gradient to afford tert-butyl (S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (0.010 g, 26% yeild) as a yellow solid. MS: [MH]⁺ 788.5.

(S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid: A mixture of tert-butyl (S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (0.070 g, 0.09 mmol) and 2,2,2-trifluoroacetic acid (0.5 mL) in anhydrous dichloromethane (2 mL) was stirred at room temperature overnight. The mixture was concentrated under reduced pressure to give a crude residue which was purified by prep-HPLC to afford (S)-2-(((6-(4,5-difluorobenzo[d]thiazol-7-yl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (0.040 g, 61% yield) as a yellow solid. ¹HNMR (400 MHz, CD₃OD): δ 9.20 (s, 1H), 7.33-7.22 (m, 3H), 6.56-6.51 (m, 1H), 4.59 (s, 2H), 4.44-3.99 (m, 3H), 3.80-3.65 (m, 5H), 3.45 (t, J=15.2 Hz, 1H), 2.81-2.71 (m, 2H), 2.46-1.92 (m, 4H), 1.74 (s, 4H), 1.62-1.40 (m, 2H), 1.16 (s, 4H). MS: [MH]⁺ 732.1.

### (S)-1-(6-benzyl-8-(hydroxymethyl)-2,6-diazaspiro[3.4]octan-2-yl)-3,3,3-trifluoro-2,2-dimethylpropan-1-one

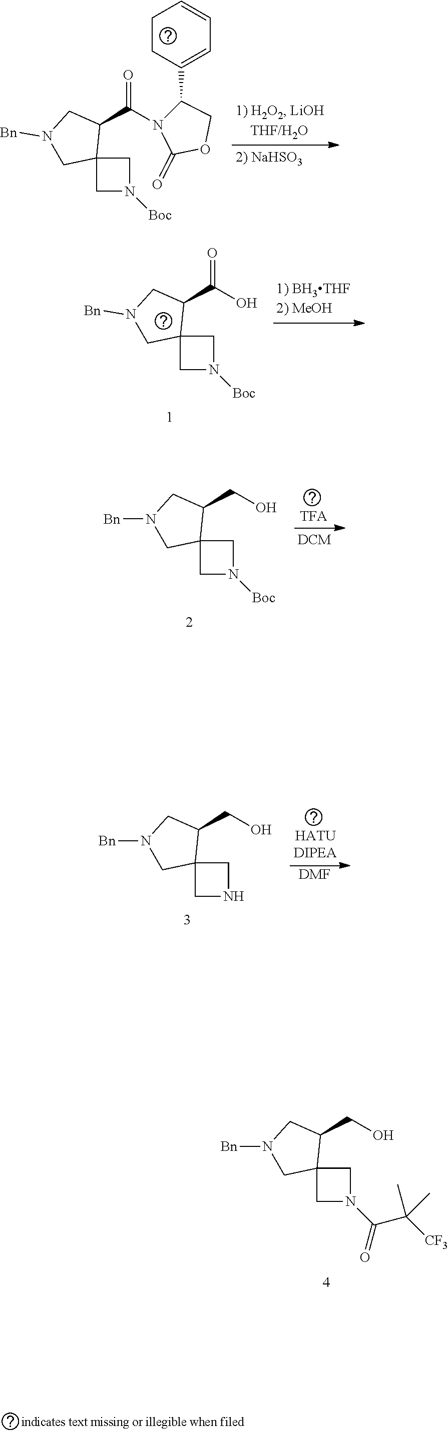

Step 1: (S)-6-benzyl-2-(tert-butoxycarbonyl)-2,6-diazaspiro[3.4]octane-8-carboxylic acid: To a solution of tert-butyl (S)-6-benzyl-8-((R)-2-oxo-4-phenyloxazolidine-3-carbonyl)-2,6-diazaspiro[3.4]octane-2-carboxylate (20 g, 40.7 mmol) was added LiOH (3.42 g, 142 mmol) in water (50 mL) and H₂O₂ (30%, 16.1 g, 13.8 mmol) at 0° C., the mixture was stirred at room temperature for 30 min. Treated with water (50 mL), the solvent was removed under reduced pressure, the aqueous phase was extracted with EtOAc (70 mL×2), the aqueous phase was carefully quenched with aq NaHSO₃ (80 mL) at 0° C., then stirred at room temperature for 18 h and extracted with DCM/MeOH=5:1 (100 mL×2), the combined organic layers were dried over Na₂SO₄, concentrated to get (S)-6-benzyl-2-(tert-butoxycarbonyl)-2,6-diazaspiro[3.4]octane-8-carboxylic acid (12.9 g, 92%) as a white solid. LCMS m/z=347.2 [M+H]⁺.

Step 2: tert-butyl (S)-6-benzyl-8-(hydroxymethyl)-2,6-diazaspiro[3.4]octane-2-carboxylate: To a solution of (S)-6-benzyl-2-(tert-butoxycarbonyl)-2,6-diazaspiro[3.4]octane-8-carboxylic acid (13.5 g, 39.0 mmol) in THF (135 mL) was added BH₃·THF (1 M in THF, 160 mL, 160 mmol) at 0° C., the mixture was stirred at rt for 1.5 h. Carefully quenched with MeOH (50 mL) and reflux for 3 h. The solvent was removed, the residue was purified by column chromatography on silica gel (eluent: DCM:MeOH=100:1˜30:1) to give the tert-butyl (S)-6-benzyl-8-(hydroxymethyl)-2,6-diazaspiro[3.4]octane-2-carboxylate (5.74 g, 44%) as a colorless oil. LCMS m/z=333.7 [M+H]⁺; ¹H NMR (400 MHz, Methanol-d₄) δ 7.39-7.16 (m, 5H), 4.19 (d, J=9.0 Hz, 1H), 3.89-3.74 (m, 2H), 3.69-3.54 (m, 5H), 2.97-2.88 (m, 2H), 2.62 (d, J=9.6 Hz, 1H), 2.30-2.23 (m, 2H), 1.42 (s, 9H).

Step 3: (S)-(6-benzyl-2,6-diazaspiro[3.4]octan-8-yl)methanol: To a solution of tert-butyl (S)-6-benzyl-8-(hydroxymethyl)-2,6-diazaspiro[3.4]octane-2-carboxylate (4.45 g, 13.4 mmol) in DCM (40 mL) was added TFA (20 mL). The reaction mixture was stirred at room temperature for 2 h. The solvent was removed under vacuum to afford crude (S)-(6-benzyl-2,6-diazaspiro[3.4]octan-8-yl)methanol (3.1 g, 100%) which was used directly in the next step. LCMS m/z=233.1 [M+H]⁺.

Step 4: (S)-1-(6-benzyl-8-(hydroxymethyl)-2,6-diazaspiro[3.4]octan-2-yl)-3,3,3-trifluoro-2,2-dimethylpropan-1-one: To a solution of 3,3,3-trifluoro-2,2-dimethylpropanoic acid (0.64 g, 4.1 mmol) in DMF (10.0 mL) was added HATU (1.57 g, 4.1 mmol) and DIPEA (4.84 g, 37.4 mmol), the mixture was stirred at room temperature for 30 min. (S)-(6-benzyl-2,6-diazaspiro[3.4]octan-8-yl)methanol (0.87 g, 3.74 mmol) in DMF (3.0 mL) was added and the reaction stirred at room temperature for another 2 h. The reaction was treated with water and extracted with EtOAc three times. The combined organic layers were washed with water, brine, dried over Na₂SO₄ and concentrated to afford (S)-1-(6-benzyl-8-(hydroxymethyl)-2,6-diazaspiro[3.4]octan-2-yl)-3,3,3-trifluoro-2,2-dimethylpropan-1-one (1.39 g, 100%) as a brown oil. LCMS m/z=371.5 [M+H]⁺; ¹H NMR (400 MHz, DMSO-d6) δ 7.37-7.14 (m, 5H), 4.70-4.57 (m, 1H), 4.31-3.98 (m, 2H), 3.85-3.66 (m, 1H), 3.66-3.39 (m, 4H), 2.86-2.71 (m, 2H), 2.53-2.50 (m, 1H), 2.24-2.07 (m, 2H), 1.32 (s, 6H).

### 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid I′-85

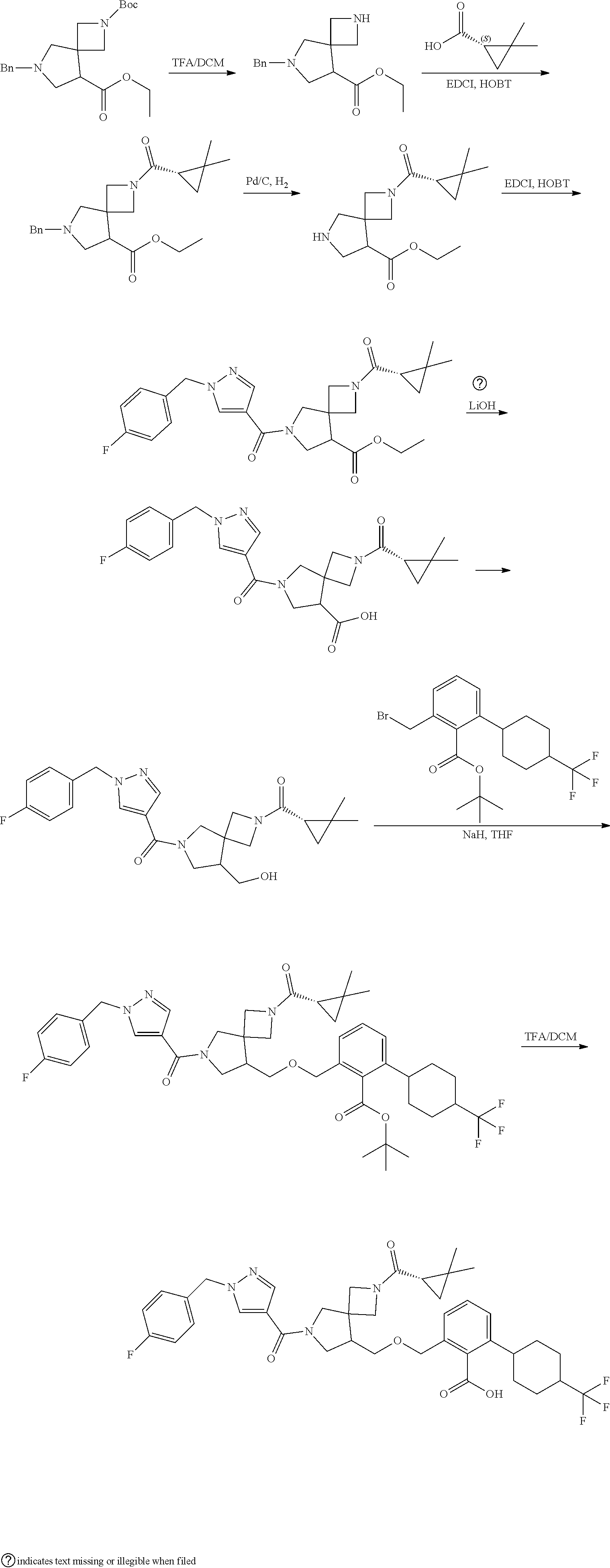

Step 1: ethyl 6-benzyl-2,6-diazaspiro[3.4]octane-8-carboxylate: To a solution of 2-(tert-butyl) 8-ethyl 6-benzyl-2,6-diazaspiro[3.4]octane-2,8-dicarboxylate (10 g, 26.7 mmol) in DCM (20 mL) was added TFA (20 mL). The reaction mixture was stirred at room temperature for 2 h. The solvent was removed under vacuum to afford crude ethyl 6-benzyl-2,6-diazaspiro[3.4]octane-8-carboxylate (7.3 g, 100%) which was used directly in the next step. LCMS m/z=275.1 [M+H]⁺.

Step 2: Ethyl 6-benzyl-2-((S)-2,2-dimethylcyclopropane-1-carbonyl)-2,6-diazaspiro[3.4]octane-8-carboxylate: To a solution of ethyl 6-benzyl-2,6-diazaspiro[3.4]octane-8-carboxylate (7.3 g, 26.7 mmol) in DCM (10 mL) was added (S)-2,2-dimethylcyclopropane-1-carboxylic acid (3.65 g, 32 mmol), EDCI (7.7 g, 40 mmol), HOBt (5.4 g, 40 mmol) and DIPEA (10 g, 80.1 mmol). The resulting mixture was stirred at room temperature for 3 h. The reaction was treated with water and extracted with EtOAc three times. The combined organic layers were washed with water, brine, dried over Na₂SO₄ and concentrated. The crude residue obtained was purified by column chromatography on silica gel (eluent: DCM:MeOH=30:1) to afford ethyl 6-benzyl-2-((S)-2,2-dimethylcyclopropane-1-carbonyl)-2,6-diazaspiro[3.4]octane-8-carboxylate (9 g, 910%) as a yellow oil. LCMS m/z=371.1 [M+H]⁺; ¹H NMR (400 MHz, Chloroform-d) δ 7.53-7.46 (m, 2H), 7.42-7.36 (m, 3H), 4.44-4.06 (m, 7H), 4.02-3.94 (m, 1H), 3.88-3.75 (m, 2H), 3.53-3.44 (m, 1H), 3.36-3.19 (m, 2H), 1.25 (t, J=7.0 Hz, 3H), 1.18-1.13 (m, 1H), 1.10-1.05 (m, 6H), 1.04-1.01 (m, 1H), 0.73-0.67 (m, 1H).

Step 3: Ethyl 2-((S)-2,2-dimethylcyclopropane-1-carbonyl)-2,6-diazaspiro[3.4]octane-8-carboxylate To a solution of ethyl 6-benzyl-2-((S)-2,2-dimethylcyclopropane-1-carbonyl)-2,6-diazaspiro[3.4]octane-8-carboxylate (9.0 g, 24.3 mmol) in EtOAc (30 mL) was added 10% Pd/C (3.6 g). The reaction mixture was stirred under a H₂ atmosphere for 24 h. The mixture was filtered through celite and concentrated to afford ethyl 2-((S)-2,2-dimethylcyclopropane-1-carbonyl)-2,6-diazaspiro[3.4]octane-8-carboxylate (6.7 g, 99%) which was used directly in the next step. LCMS m/z=281.0 [M+H]⁺.

Step 4: ethyl 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylate To a solution of ethyl 2-((S)-2,2-dimethylcyclopropane-1-carbonyl)-2,6-diazaspiro[3.4]octane-8-carboxylate (6.8 g, 24.3 mmol) in DCM (20 mL) was added 1-(4-fluorobenzyl)-1H-pyrazole-4-carboxylic acid (6.4 g, 29.2 mmol), EDCI (7.0 g, 36.4 mmol), HOBt (4.9 g, 36.4 mmol) and DIPEA (9.4 g, 72.9 mmol). The resulting mixture was stirred for 3 h. The reaction was treated with water and extracted with EtOAc three times. The combined organic layers were washed with water, brine, dried over Na₂SO₄ and concentrated. The crude residue obtained was purified by column chromatography on silica gel (eluent: DCM:MeOH=20:1) to afford ethyl 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylate (5.8 g, 49%) as a white solid. LCMS m/z=483.1 [M+H]⁺; ¹H NMR (400 MHz, Chloroform-d) δ 7.87-7.76 (m, 2H), 7.26-7.20 (m, 2H), 7.04 (t, J=8.4 Hz, 2H), 5.27 (s, 2H), 4.29-4.17 (m, 3H), 4.08-4.04 (m, 1H), 4.02-3.64 (m, 6H), 3.24-3.12 (m, 1H), 1.30-1.26 (m, 3H), 1.23-1.18 (m, 1H), 1.15-1.12 (m, 7H), 0.79-0.72 (m, 1H).

Step 5: 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylic acid To a solution of ethyl 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylate (5.8 g, 12 mmol) in a mixture of THF, EtOH and H₂O (28 mL/7 mL/7 mL) at 0° C. was added lithium hydroxide monohydrate (866 mg, 32 mmol). The reaction mixture was stirred at 0° C. for 1 h, diluted with water (40 mL) and extracted with EtOAc (70 mL). The aqueous layer was collected, acidified with 1 M HCl to pH˜2 and extracted with EtOAc (70 mL×3). The combined organic layers were washed with brine, dried over Na₂SO₄, filtered and concentrated to afford crude 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylic acid (5 g, 91%) as a white solid which was used directly in the next step. LCMS m/z=455.1 [M+H]⁺; ¹H NMR (400 MHz, Chloroform-d) δ 7.90-7.87 (m, 1H), 7.82 (d, J=8.0 Hz, 1H), 7.26-7.21 (m, 2H), 7.04 (t, J=8.4 Hz, 2H), 5.27 (s, 2H), 4.45-4.32 (m, 2H), 4.12-3.80 (m, 6H), 3.25-3.14 (m, 1H), 1.23-1.19 (m, 1H), 1.17-1.10 (m, 7H), 0.80-0.73 (m, 1H).

Step 6: (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone A solution of 2-((S)-2,2-dimethylcyclopropane-1-carbonyl)-6-(1-(4-fluorobenzyl)-1H-pyrazole-4-carbonyl)-2,6-diazaspiro[3.4]octane-8-carboxylic acid (5 g, 11 mmol) isobutyl carbonochloridate (2.2 g, 15.9 mmol) and 4-methylmorpholine (1.49 g, 14.7 mmol) in THF (35 mL) was stirred at 0° C. for 0.5 h. NaBH₄ dissolved in H₂O (20 mL) was added to the solution at 0° C. and the mixture was stirred for another 2 h at the same temperature. The reaction was treated with water (40 mL) and extracted with EtOAc (70 mL×3) three times. The combined organic layers were dried over Na₂SO₄ and concentrated. The crude residue obtained was purified by column chromatography on silica gel to afford (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (3.3 g, 68%) as a white solid LCMS m/z=441.1 [M+H]⁺; ¹H NMR (400 MHz, Chloroform-d) δ 7.88-7.76 (m, 2H), 7.26-7.21 (m, 2H), 7.05 (t, J=8.4 Hz, 2H), 5.27 (s, 2H), 4.53-4.11 (m, 2H), 4.07-3.94 (m, 2H), 3.92-3.79 (m, 4H), 3.77-3.53 (m, 2H), 2.57-2.40 (m, 1H), 2.34-2.16 (m, 1H), 1.24-1.20 (m, 1H), 1.17-1.13 (m, 6H), 0.78-0.72 (m, 1H).

Step 7: tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoate To a solution of (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (300 mg, 0.68 mmol) and tert-butyl 2-(bromomethyl)-6-(4-(trifluoromethyl)cyclohexyl)benzoate (320 mg, 0.75 mmol) in THF at 0° C. was added sodium hydride (42 mg, 1.0 mmol), Then the mixture was warmed to room temperature and stirred at RT overnight. The mixture was diluted with water (30 mL) and extracted with EtOAc (70 mL×2). The combined organic layers were dried over Na₂SO₄, filtered and concentrated. The mixture was purified by prep-TLC to afford tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoate (220 mg, 41% yield) as a glass solid. LCMS m/z=781.4 [M+H]⁺; ¹H NMR (400 MHz, DMSO-d₆) δ 8.33 (d, J=8.0 Hz, 1H), 7.85-7.78 (m, 1H), 7.37-7.14 (m, 7H), 5.34 (s, 2H), 4.53-4.44 (m, 2H), 4.36-3.40 (m, 12H), 2.03-1.82 (m, 2H), 1.72-1.41 (m, 14H), 1.39-1.21 (m, 2H), 1.19-0.92 (m, 7H), 0.88-0.83 (m, 1H), 0.68-0.62 (m, 1H).

Step 8: 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid I′-85 To a solution of tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoate (195 mg, 0.25 mmol) in DCM (4 mL) was added TFA (2 mL). The reaction mixture was stirred at room temperature for 2 h. The solvent was removed under vacuum and purified by prep-HPLC to afford 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid (140 mg, 77% yield) as a white solid. LCMS m/z=725.3 [M+H]⁺ ; 1H NMR (400 MHz, DMSO-d6) δ 13.12 (s, 1H), 8.35 (s, 1H), 7.83 (d, J=8.4 Hz, 1H), 7.37-7.13 (m, 7H), 5.34 (s, 2H), 4.57-4.41 (m, 2H), 4.25-3.37 (m, 11H), 2.77-2.55 (m, 2H), 2.16-1.21 (m, 9H), 1.13-0.96 (m, 6H), 0.88-0.83 (m, 1H), 0.69-0.62 (m, 1H).

### 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzamide I′-84

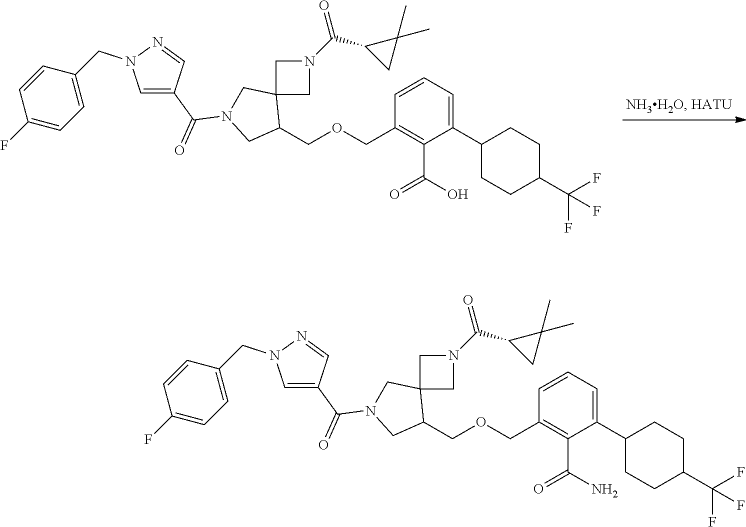 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(=O)O)C1 ; CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(N)=O)C1]

To a solution of 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzoic acid (70 mg, 0.097 mmol) in DMF (1 mL) was added HATU (37 mg, 0.097 mmol) and DIPEA (38 mg, 0.291 mmol), the mixture was stirred at room temperature for 30 min. Ammonium hydroxide (0.5 mL) was added and the reaction stirred at room temperature for another 2 h. The crude reaction solution was purified by RP column (C18, 20 g, 40% ACN in water) to afford 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzamide (mg, 85%) as a white solid. LCMS m/z=724.3 [M+H]⁺; ¹H NMR (400 MHz, CDCl₃) δ 7.87-7.67 (m, 2H), 7.32-7.27 (m, 2H), 7.26-7.14 (m, 3H), 7.05 (m, 2H), 5.27 (s, 2H), 4.60-4.48 (m, 2H), 4.33-3.59 (m, 10H), 2.88-2.48 (m, 2H), 2.46-2.30 (m, 1H), 2.17-1.97 (m, 3H), 1.74-1.70 (m, 5H), 1.53-1.43 (m, 1H), 1.15-1.04 (m, 7H), 1.01-0.50 (m, 1H).

### 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid I-71

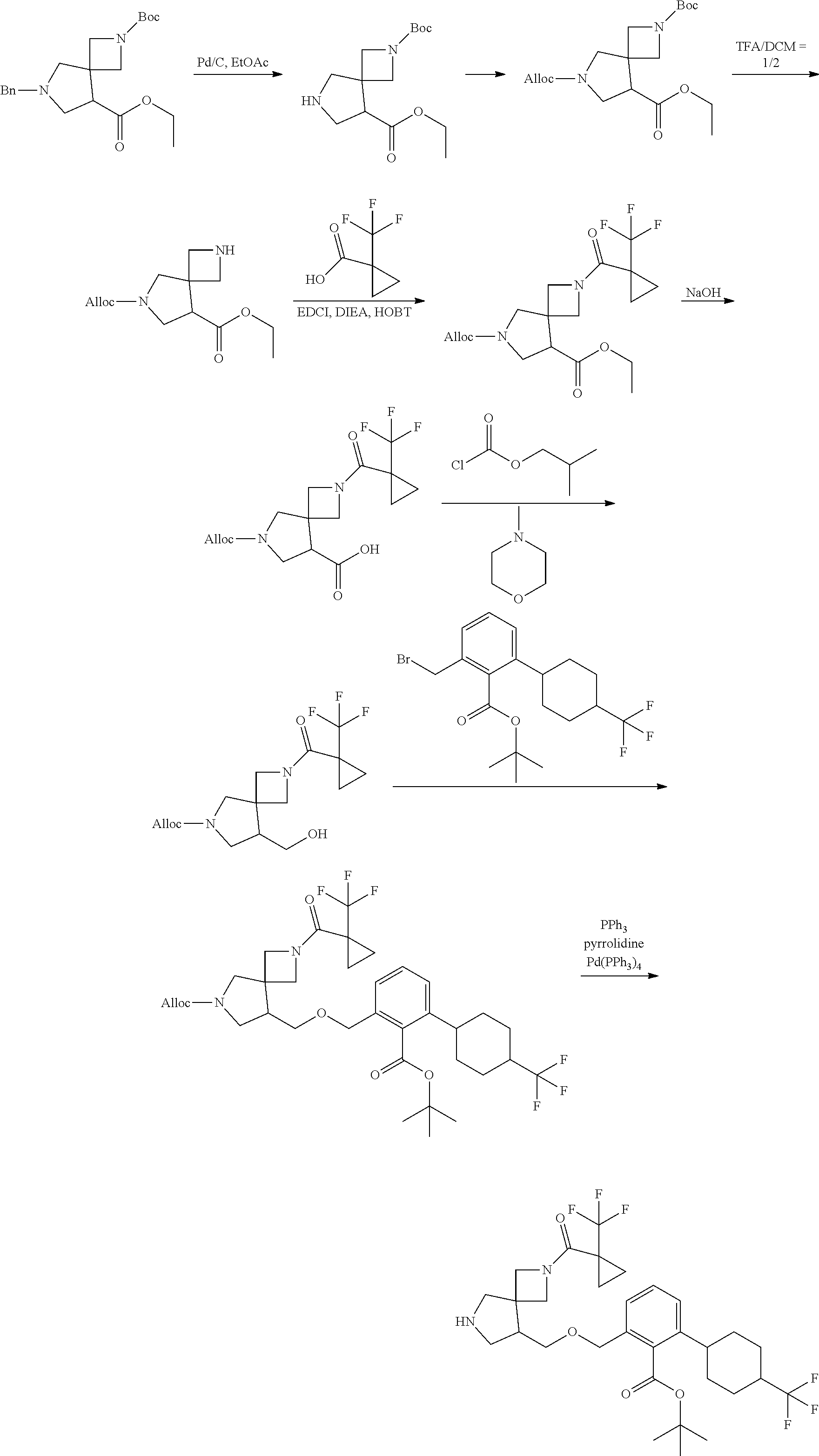

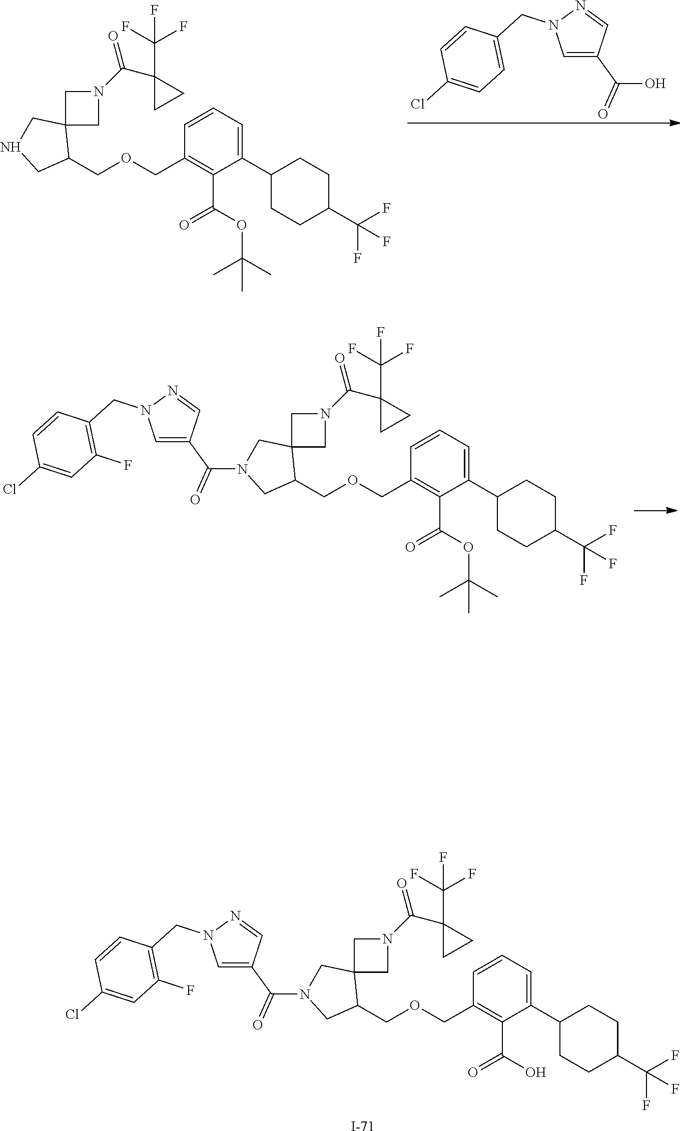

Step 1: 2-(tert-butyl) 8-ethyl 2,6-diazaspiro[3.4]octane-2,8-dicarboxylate: To a solution of 2-(tert-butyl) 8-ethyl 6-benzyl-2,6-diazaspiro[3.4]octane-2,8-dicarboxylate (5.0 g, 13.35 mmol) in EtOAc (40 mL) was added 10% Pd/C (2.5 g). The reaction mixture was stirred at 45° C. for 36 h under an atmosphere of H₂. The mixture was filtered and concentrated to afford 2-(tert-butyl) 8-ethyl 2,6-diazaspiro[3.4]octane-2,8-dicarboxylate (3.8 g, 100%) as a colorless oil which was used directly in the next step. LCMS m/z=285.3 [M+H]⁺.

Step 2: 6-allyl 2-(tert-butyl) 8-ethyl 2,6-diazaspiro[3.4]octane-2,6,8-tricarboxylate: To a solution of 2-(tert-butyl) 8-ethyl 2,6-diazaspiro[3.4]octane-2,8-dicarboxylate (7.6 g, 26.73 mmol) in DCM (70 mL) was added TEA (8.1 g, 80.19 mmol) and AllocCl (3.2 g, 26.73 mmol) at 0° C. The reaction mixture was stirred at room temperature for 3 h. The mixture was diluted with water (150 mL) and extracted with DCM (200 mL×3). The organic layers were washed with brine, dried over Na₂SO₄, filtered and concentrated. The crude was purified by column (Pet. Ether:EtOAc=5/1) to afford 6-allyl 2-(tert-butyl) 8-ethyl 2,6-diazaspiro[3.4]octane-2,6,8-tricarboxylate (13.5 g, 71.6%) as a yellow oil. ¹H NMR (400 MHz, DMSO-d6) δ 5.99-5.84 (m, 1H), 5.34-5.22 (m, 1H), 5.23-5.14 (m, 1H), 4.55-4.48 (m, 2H), 4.21-4.05 (m, 2H), 3.99-3.91 (m, 1H), 3.83-3.71 (m, 3H), 3.63-3.45 (m, 4H), 3.30-3.24 (m, 1H), 1.37 (s, 9H), 1.19 (t, J=7.2 Hz, 3H).

Step 3: 6-allyl 8-ethyl 2,6-diazaspiro[3.4]octane-6,8-dicarboxylate: To a solution of 6-allyl 2-(tert-butyl) 8-ethyl 2,6-diazaspiro[3.4]octane-2,6,8-tricarboxylate (500 mg, 1.36 mmol) in DCM (4 mL) was added TFA (2 mL). The reaction mixture was stirred at room temperature for 1 h. The solvent was removed under vacuum to afford 6-allyl 8-ethyl 2,6-diazaspiro[3.4]octane-6,8-dicarboxylate (364 mg, 100%) as a yellow oil which was used directly in the next step. LCMS m/z=269.2 [M+H]⁺.

Step 4: 6-allyl 8-ethyl 2-(1-(trifluoromethyl)cyclopropane-1-carbonyl)-2,6-diazaspiro[3.4]octane-6,8-dicarboxylate: To a solution of 1-(trifluoromethyl)cyclopropane-1-carboxylic acid (830 mg, 5.40 mmol) in DCM (15 mL) was added EDCI (1.55 g, 8.10 mmol), HOBt (1.1 g, 8.10 mmol) and DIPEA (4.18 g, 32.40 mmol). The mixture was stirred at room temperature for 30 min. 6-allyl 8-ethyl 2,6-diazaspiro[3.4]octane-6,8-dicarboxylate (1.45 g, 5.40 mmol) was added. The reaction mixture was stirred at room temperature for 3 h. The mixture was diluted with water (30 mL) and extracted with DCM (50 mL×3). The combined organic layers were washed with brine, dried over Na₂SO₄, filtered and concentrated. The residue obtained was purified by column to afford 6-allyl 8-ethyl 2-(1-(trifluoromethyl)cyclopropane-1-carbonyl)-2,6-diazaspiro[3.4]octane-6,8-dicarboxylate (1.4 g, 64.2%) as a yellow oil. LCMS m/z=405.1[M+H]⁻; ¹H NMR (400 MHz, DMSO-d6) δ 5.98-5.85 (m, 1H), 5.33-5.24 (m, 1H), 5.21-5.16 (m, 1H), 4.57-4.49 (m, 2H), 4.44-4.05 (m, 5H), 3.87 (s, 1H), 3.68-3.48 (m, 4H), 3.41-3.34 (m, 1H), 1.22-1.07 (m, 7H).

Step 5: 6-((allyloxy)carbonyl)-2-(1-(trifluoromethyl)cyclopropane-1-carbonyl)-2,6-diazaspiro[3.4]octane-8-carboxylic acid: To a solution of 6-allyl 8-ethyl 2-(1-(trifluoromethyl)cyclopropane-1-carbonyl)-2,6-diazaspiro[3.4]octane-6,8-dicarboxylate (150 mg, 0.371 mmol) in a mixture of THF and H₂O and EtOH (2 mL/0.5 mL/0.5 mL) was added NaOH (30 mg, 0.742 mmol). The reaction mixture was stirred at room temperature for 2 h. The mixture was diluted with water (20 mL) and extracted with EtOAc (30 mL×3). The aqueous layer was collected and acidified with 1 M HCl to pH˜2 and extracted with EtOAc (30 mL×3). The combined organic layers were washed with brine, dried over Na₂SO₄, filtered and concentrated to afford 6-((allyloxy)carbonyl)-2-(1-(trifluoromethyl)cyclopropane-1-carbonyl)-2,6-diazaspiro[3.4]octane-8-carboxylic acid (100 mg, 72%) as a colourless oil. LCMS m/z=377.1[M+H]⁺; ¹H NMR (400 MHz, DMSO-d6) δ 12.77 (s, 1H), 6.01-5.84 (m, 1H), 5.33-5.23 (m, 1H), 5.18 (dd, J=10.6, 1.8 Hz, 1H), 4.51 (d, J=5.2 Hz, 2H), 4.42-4.10 (m, 2H), 4.00-3.76 (m, 2H), 3.65-3.45 (m, 4H), 3.29-3.19 (m, 1H), 1.26-1.13 (m, 4H).

Step 6: allyl 8-(hydroxymethyl)-2-(1-(trifluoromethyl)cyclopropane-1-carbonyl)-2,6-diazaspiro[3.4]octane-6-carboxylate: To a solution of 6-((allyloxy)carbonyl)-2-(1-(trifluoromethyl)cyclopropane-1-carbonyl)-2,6-diazaspiro[3.4]octane-8-carboxylic acid (100 mg, 0.266 mmol) in THE (1.0 mL) was added 4-methylmorpholine (35 mg, 0.345 mmol) and isobutyl carbonochloridate (51 mg, 0.372 mmol) at 0° C. for 0.5 h. Then NaBH₄ (30 mg, 0.797 mmol) in H₂O (0.5 mL) was added and the mixture was stirred at 0° C. for another 2 h. The mixture was diluted with water (20 mL) and extracted with EtOAc (30 mL×3). The organic layers were washed with brine, dried over Na₂SO₄, filtered and concentrated. The crude was purified by column (DCM/MEOH=35/1) to afford allyl 8-(hydroxymethyl)-2-(1-(trifluoromethyl)cyclopropane-1-carbonyl)-2,6-diazaspiro[3.4]octane-6-carboxylate (35 mg, 36.5%) as a colourless oil. LCMS m/z=363.1[M+H]⁺; ¹H NMR (400 MHz, DMSO-d6) δ 5.98-5.86 (m, 1H), 5.28 (d, J=17.2 Hz, 1H), 5.22-5.16 (m, 1H), 4.76 (t, J=4.8 Hz, 1H), 4.53-4.48 (m, 2H), 4.32-3.98 (m, 2H), 3.86-3.76 (m, 1H), 3.74-3.65 (m, 1H), 3.60-3.50 (m, 3H), 3.47-3.36 (m, 2H), 3.25-3.13 (m, 1H), 2.88-2.71 (m, 1H), 1.22-1.12 (m, 4H).

Step 7: allyl 8-(((2-(tert-butoxycarbonyl)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carboxylate: To a solution of allyl 8-(hydroxymethyl)-2-(1-(trifluoromethyl)cyclopropane-1-carbonyl)-2,6-diazaspiro[3.4]octane-6-carboxylate (550 mg, 1.52 mmol) in THF (6 mL) at 0° C. was added NaH (152 mg, 3.80 mmol). The mixture was stirred at 0° C. for 0.5 h, then tert-butyl 2-(bromomethyl)-6-(4-(trifluoromethyl)cyclohexyl)benzoate (766 mg, 1.82 mmol) was added. The mixture was stirred at room temperature overnight. The mixture was diluted with water (20 mL) and extracted with EtOAc (30 mL×3). The combined organic layers were washed with brine, dried over Na₂SO₄ and concentrated. The crude was purified by column to afford allyl 8-(((2-(tert-butoxycarbonyl)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carboxylate (360 mg, 33.7%) as a colorless oil. LCMS m/z=725.3[M+Na]*.

Step 8: tert-butyl 2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate: To a solution of allyl 8-(((2-(tert-butoxycarbonyl)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carboxylate (400 mg, 0.569 mmol) in DCM (4 mL) was added pyrrolidine (202 mg, 2.85 mmol), PPh₃ (38 mg, 0.142 mmol) and Pd(PPh₃)₄ (65 mg, 0.057 mmol). The mixture was stirred at room temperature for 2 h. The mixture was diluted with water (20 mL) and extracted with DCM (30 mL×3). The combined organic layers were washed with brine, dried over Na₂SO₄ and concentrated. The crude was purified by column to afford tert-butyl 2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate (220 mg, 62.5%) as a yellow oil. LCMS m/z=619.4[M+H]⁺; ¹H NMR (400 MHz, Chloroform-d) δ 7.36-7.30 (m, 1H), 7.25-7.16 (m, 2H), 4.50 (s, 2H), 4.29-3.91 (m, 3H), 3.56-3.46 (m, 2H), 3.25-3.05 (m, 3H), 2.81-2.61 (m, 2H), 2.44-2.29 (m, 2H), 2.18-1.99 (m, 4H), 1.97-1.73 (m, 12H), 1.72-1.63 (m, 2H), 1.17-1.04 (m, 4H).

Step 9: tert-butyl 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate To a solution of 1-(4-chloro-2-fluorobenzyl)-1H-pyrazole-4-carboxylic acid (36 mg, 0.15 mmol), HATU (54 mg, 0.15 mmol) and tert-butyl 2-(4-(trifluoromethyl)cyclohexyl)-6-(((2-(1-(trifluoromethyl)cyclopropane-1-carbonyl)-2,6-diazaspiro[3.4]octan-8-yl)methoxy)methyl)benzoate (90 mg, 0.15 mmol) in DCM (10 mL) was added DIEA (56 mg, 0.15 mmol) and the mixture stirred at room temperature for 2 h. The mixture was diluted with water (20 mL) and extracted with DCM (30 mL×2). The combined organic layers were washed with brine, dried over Na₂SO₄, filtered and concentrated. The residue obtained was purified by column chromatography on silica gel (eluent: Pet. Ether:EtOAc=6:1) to afford tert-butyl 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (80 mg, 73%) as a yellow oil. LCMS m/z=855.2 [M+H]⁺; ¹H NMR (400 MHz, CDCl₃) δ=7.98-7.74 (m, 2H), 7.30 (d, J=7.6, 2H), 7.14 (s, 4H), 5.33 (s, 2H), 4.53 (d, J=11.0, 2H), 4.39-3.70 (m, 8H), 3.57 (s, 2H), 3.46 (s, 1H), 2.80 (s, 1H), 2.10-2.06 (m, 4H), 1.76 (s, 4H), 1.57 (s, 9H), 1.42 (d, J=5.2, 1H), 1.17 (d, J=8.9, 4H).

Step 10: 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid 1-71 To a solution of tert-butyl 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoate (90 mg, 0.11 mmol) in DCM (4 mL) was added TFA (2 mL). The reaction mixture was stirred at room temperature for 1 h. The solvent was removed under vacuum to afford 2-(((6-(1-(4-chloro-2-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzoic acid (I-71), 30 mg, 36%). LCMS m/z=799.9 [M+H]⁺; ¹H NMR (400 MHz, DMSO-d₆) δ 8.32 (s, 1H), 7.82 (d, J=7.8 Hz, 1H), 7.47 (d, J=10.0 Hz, 1H), 7.34-7.24 (m, 3H), 7.22 (d, J=7.7 Hz, 2H), 5.41 (s, 2H), 4.49 (s, 2H), 4.01-3.87 (m, 2H), 3.86-3.67 (m, 2H), 3.65-3.59 (m, 1H), 3.58-3.44 (m, 2H), 3.29 (s, 3H), 2.78-2.59 (m, 2H), 2.03-1.91 (m, 2H), 1.67 (s, 5H), 1.24 (s, 4H).

TABLE 11
The compounds listed in Table 11 were synthesized from tert-butyl 2-(4-
(trifluoromethyl)cyclohexyl)-6-(((2-(1-(trifluoromethyl)cyclopropane-1-carbonyl)-2,6-
diazaspiro[3.4]octan-8-yl)methoxy)methyl)benzoate according to the procedures outlined for I-71
using the appropriate commercially available reagents and/or intermediates described elsewhere.

| Cmpd. | | | |
|---|---|---|---|
| No. | Compound | ¹HNMR | LCMS |
| I-76 | 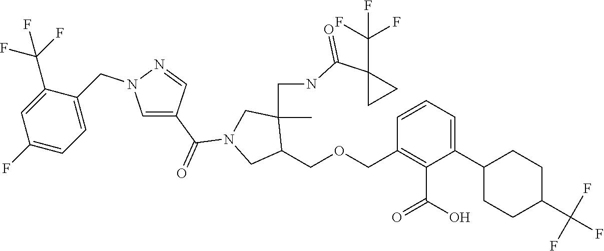 [SMILES: CC1(CNC(=O)C2(C(F)(F)F)CC2)CN(C(=O)c2cnn(Cc3ccc(F)cc3C(F)(F)F)c2)CC1COCc1cccc(C2CCC(C(F)(F)F)CC2)c1C(=O)O] | ¹H NMR (400 MHz, DMSO-d6) δ 8.36 (d, J = 10.2 Hz, 1H), 7.88 (d, J = 8.8 Hz, 1H), 7.69 (d, J = 8.8 Hz, 1H), 7.57-7.48 (m, 1H), 7.34-7.16 (m, 3H), 7.09 (s, 1H), 5.55 (s, 2H), 4.49 (s, 2H), 4.11- 3.35 (m, 12H), 2.78- 2.55 (m, 3H), 1.95 (s, 2H), 1.65 (s, 4H), 1.12 (s, 4H). | m/z = 833.3 [M + H]⁺ |

### (8-(((2-(2H-tetrazol-5-yl)-3-(4-(trifluoromethyl)cyclohexyl)benzyl)oxy)methyl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone

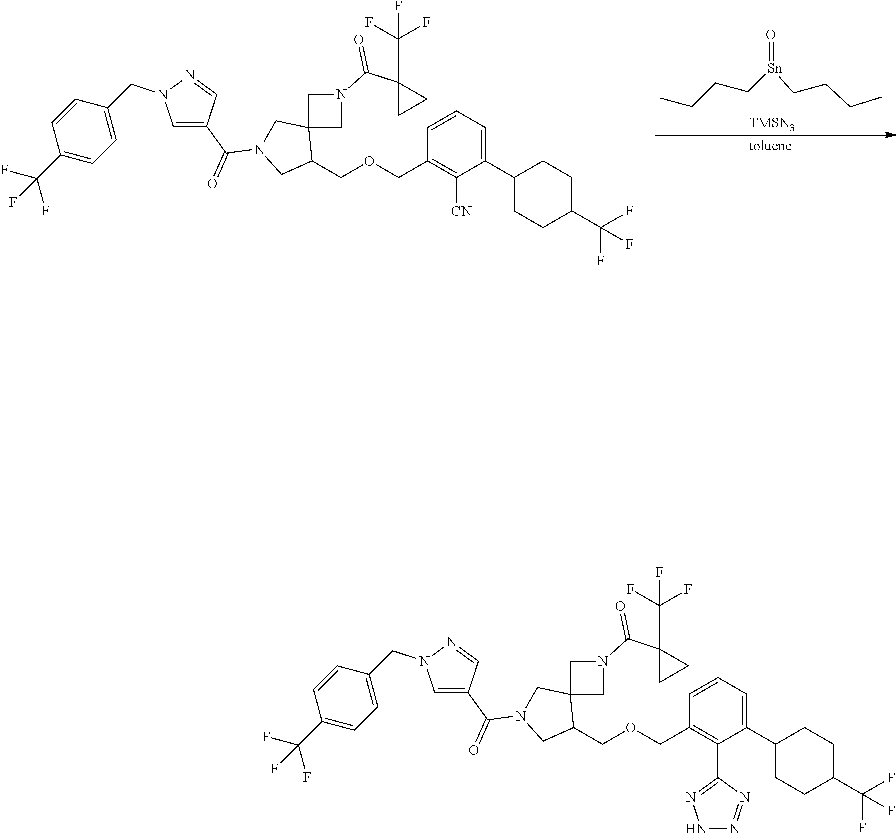

To a solution of 2-(((6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)-6-(4-(trifluoromethyl)cyclohexyl)benzonitrile (60 mg, 0.075 mmol) was added dibutylstannanone (7.5 mg, 0.03 mmol) and TMSN₃ (26 mg, 0.23 mmol), the mixture was heated to 130° C. in seal tube for 5 h. Treated with water and xetracted with EtOAc (10 mL×2). The combined organic layers were washed with brine, dried over Na₂SO₄, filtered and concentrated. The residue obtained was purified by prep-TLC (eluent: DCM:MeOH=15:1) to afford (8-(((2-(2H-tetrazol-5-yl)-3-(4-(trifluoromethyl)cyclohexyl)benzyl)oxy)methyl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (20 mg, 31%) as a white solid. LCMS m/z=839.4 [M+H]⁺; ¹H NMR (400 MHz, Methanol-d₄) δ 8.49-8.21 (m, 1H), 8.02-7.84 (m, 1H), 7.70-7.58 (m, 2H), 7.58-7.28 (m, 5H), 5.51 (d, J=20.8 Hz, 2H), 4.27 (s, 4H), 3.98 (q, J=10.4 Hz, 2H), 3.90-3.56 (m, 3H), 3.56-3.37 (m, 3H), 2.51-1.45 (m, 11H), 1.20 (s, 4H).

### 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N-(methylsulfonyl)-6-(4-(trifluoromethyl)cyclohexyl)benzamide I-86

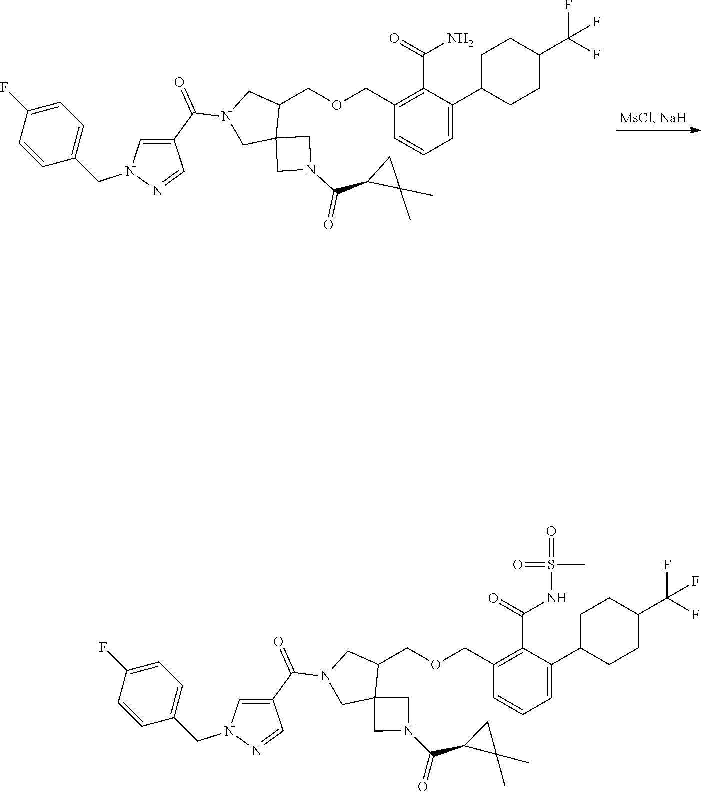 [SMILES: CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(N)=O)C1 ; CC1(C)C[C@@H]1C(=O)N1CC2(CN(C(=O)c3cnn(Cc4ccc(F)cc4)c3)CC2COCc2cccc(C3CCC(C(F)(F)F)CC3)c2C(=O)NS(C)(=O)=O)C1]

To a solution of 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzamide (70 mg, 0.097 mmol) in THF (2 mL) was added NaH (8 mg, 0.19 mmol) and stirred for 30 min at 0° C. Methanesulfonyl chloride was added to the solution at 0° C. Then the mixture was warmed to room temperature and stirred overnight. The mixture was diluted with water (30 mL) and extracted with EtOAc (50 mL×3). The combined organic layers were washed with brine, dried over Na₂SO₄, filtered and concentrated. The residue obtained was purified by prep-HPLC to afford 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N-(methylsulfonyl)-6-(4-(trifluoromethyl)cyclohexyl)benzamide (1-86, 8 mg, 10% yield) as a white solid. LCMS m/z=800.3 [M−H]⁻; ¹H NMR (400 MHz, CDCl₃) δ 7.85-7.76 (m, 1H), 7.66 (m, 1H), 7.36-7.27 (m, 3H), 7.24-7.20 (m, 2H), 7.10-7.02 (m, 2H), 5.25 (s, 2H), 4.57-3.67 (m, 13H), 3.44 (s, 3H), 2.78-2.34 (m, 4H), 2.21-2.07 (m, 3H), 1.81-1.69 (m, 5H), 1.36-1.24 (m, 1H), 1.18-0.95 (m, 6H).

### 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoic acid I-118

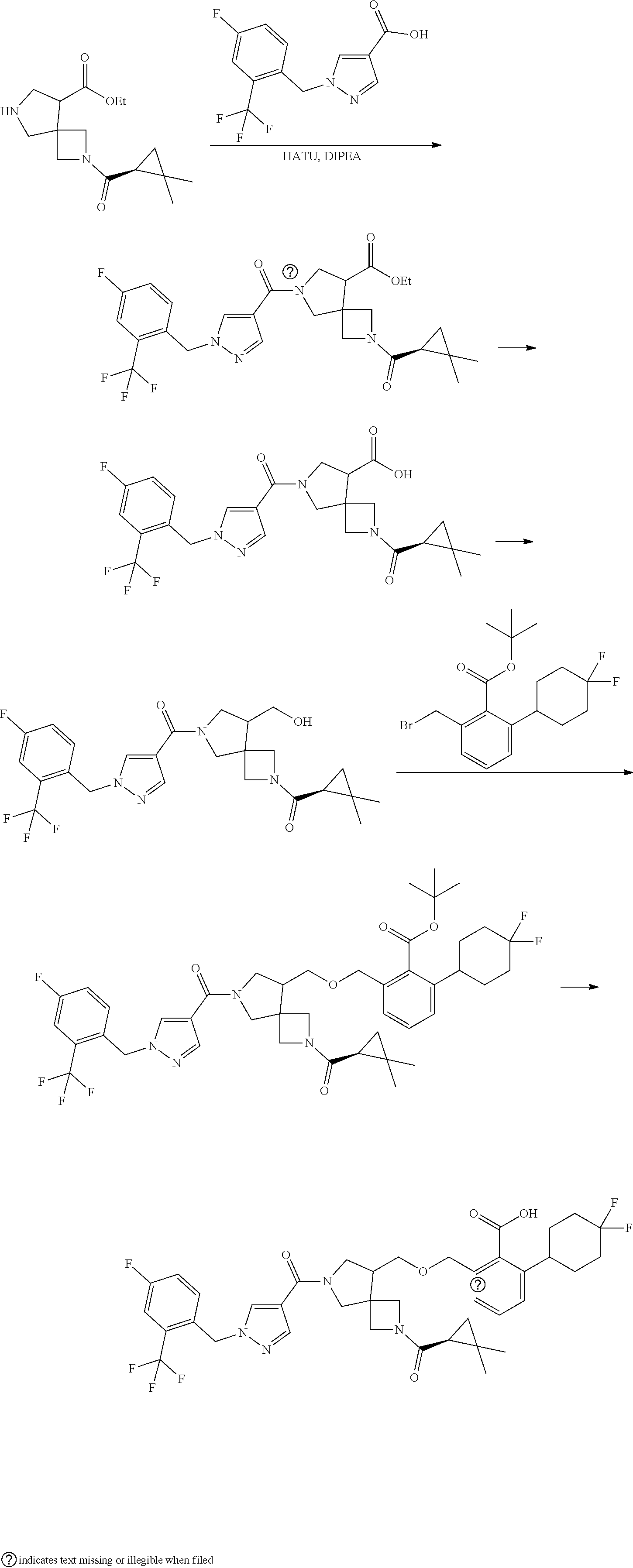

Step 1: ethyl 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane- 8-carboxylate: To a solution of 1-(4-fluoro-2-(trifluoromethyl)benzyl)-1H-pyrazole-4-carboxylic acid (452 mg, 1.57 mmol) in DCM (5 mL) was added HATU (597 mg, 1.57 mmol) and DIPEA (553 mg, 4.28 mmol). After stirring for 15 min, ethyl 2-((S)-2,2-dimethylcyclopropane-1-carbonyl)-2,6-diazaspiro[3.4]octane-8-carboxylate (400 mg, 1.43 mmol) was added and the reaction stirred at room temperature for another 3 h. The mixture was diluted with water (20 mL) and extracted with DCM (25 mL×2). The combined organic layers were washed with water, brine, dried over Na₂SO₄, filtered and concentrated. The crude was purified by column chromatography on silica gel (eluent: DCM:MeOH=30:1) to afford ethyl 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane-8-carboxylate (488 mg, 62%) as a white solid. LCMS m, z=551.5 [M+H]⁻.

Step 2: 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane-8-carboxylic acid a solution of ethyl 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane-8-carboxylate (510 mg, 0.93 mmol) in MeOH (4 mL) was added 10% NaOH (1 mL). The resulting mixture was stirred at room temperature for 2 h. The mixture was acidified with 1 M HCl to pH˜3 and extracted with EtOAc (30 mL×3). The combined organic layers were washed with brine, dried over Na₂SO₄, filtered and concentrated to afford 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane-8-carboxylic acid (350 mg, 72%) as a white solid. LCMS m/z=521.4 [M−H]⁻.

Step 3: (2-((S)-2,2-dimethylcyclopropane-1-carbonyl)-8-(hydroxymethyl)-2,6-diazaspiro[3.4]octan-6-yl)(1-(4-fluoro-2-(trifluoromethyl)benzyl)-1H-pyrazol-4-yl)methanone To a solution of 2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octane-8-carboxylic acid (254 mg, 0.48 mmol) in THF (3 mL) at 0° C. was added 4-methylmorpholine (64 mg, 0.64 mmol) and isobutyl carbonochloridate (94 mg, 0.69 mmol). The mixture was stirred at 0° C. for 30 min. NaBH₄ (54 mg, 1.44 mmol) dissolved in water (0.5 mL) was added. The mixture was stirred at room temperature for another 1.5 h. The mixture was diluted with water (30 mL) and extracted with EtOAc (50 mL×3). The combindorganic layers were washed with brine, dried over Na₂SO₄, filtered and concentrated. The residue obtained was purified by column chromatography on silica gel (eluent: DCM. MeOH=40:1) to afford (2-((S)-2,2-dimethylcyclopropane-1-carbonyl)-8-(hydroxymethyl)-2,6-diazaspiro[3.4]octan-6-yl)(1-(4-fluoro-2-(trifluoromethyl)benzyl)-1H-pyrazol-4-yl)methanone (296 mg, 87%) as a white solid. LCMS m/z=509.3 [M+H]⁻; HNMR (400 MHz, CD₃OD) δ 8.22 (d, 1H), 8.01-7.92 (m, 1H), 7.56 (dd, 1H), 7.43-7.29 (m, 1H), 7.21-7.06 (m, 1H), 5.59 (s, 2H), 4.60-4.53 (m, 1H), 4.40-3.52 (m, 9H), 2.60-2.44 (m, 1H), 1.33-1.27 (m, 4H), 1.21-1.18 (m, 3H), 1.15-1.12 (m, 2H).

Step 4: tert-butyl 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoate To a solution of (2-((S)-2,2-dimethylcyclopropane-1-carbonyl)-8-(hydroxymethyl)-2,6-diazaspiro[3.4]octan-6-yl)(1-(4-fluoro-2-(trifluoromethyl)benzyl)-1H-pyrazol-4-yl)methanone (50 mg, 0.1 mmol) in dry THF (0.5 mL) at 0° C. was added NaH (12 mg, 0.20 mmol). The mixture was stirred at 0° C. for 30 min. tert-butyl 2-(bromomethyl)-6-(4,4-difluorocyclohexyl)benzoate (48 mg, 0.12 mmol) was added and the resulting reaction mixture was stirred overnight, then diluted with water (10 mL) and extracted with EtOAc (30 mL). The combined organic layers were washed with brine, dried over Na₂SO₄, filtered and concentrated. The mixture was purified by prep-TLC (eluent: DCM:MeOH=15:1) to afford tert-butyl 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoate (55 mg, 67%) as a white solid. LCMS m/z=817.4 [M+H]⁺; ¹HNMR (400 MHz, CD₃OD) δ 8.29-8.18 (m, 1H), 8.03-7.92 (m, 1H), 7.63-7.52 (m, 1H), 7.45-7.09 (m, 5H), 5.62 (s, 2H), 4.66-4.58 (m, 2H), 4.32-3.64 (m, 10H), 2.88-2.75 (m, 1H), 2.68-2.60 (m, 1H), 2.22-1.79 (m, 8H), 1.67-1.56 (m, 9H), 1.23-1.02 (m, 8H), 0.83-0.72 (m, 1H).

Step 5: 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoic acid I-118 To a solution of tert-butyl 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoate (90 mg, 0.11 mmol) in DCM (1 mL) was added TFA (0.5 mL). The reaction mixture was stirred at room temperature for 2 h. The mixture was purified by prep-HPLC to afford 2-(4,4-difluorocyclohexyl)-6-(((2-((S)-2,2-dimethylcyclopropane-1-carbonyl)-6-(1-(4-fluoro-2-(trifluoromethyl)benzyl)-1H-pyrazole-4-carbonyl)-2,6-diazaspiro[3.4]octan-8-yl)methoxy)methyl)benzoic acid (40 mg, 39%) as a white solid. LCMS m/z=761.3 [M+H]⁺; ¹HNMR (400 MHz, CD₃OD) δ 8.31-8.17 (m, 1H), 8.01-7.92 (m, 1H), 7.54 (d, J=9.0 Hz, 1H), 7.40-7.19 (m, 4H), 7.17-7.07 (m, 1H), 5.59 (s, 2H), 4.67-3.54 (m, 12H), 2.90-2.77 (m, 1H), 2.75-2.56 (m, 1H), 2.18-2.05 (m, 2H), 1.96-1.70 (m, 6H), 1.46-1.29 (m, 1H), 1.20-0.99 (m, 7H), 0.80-0.69 (m, 1H).

### tert-butyl 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate I′-79

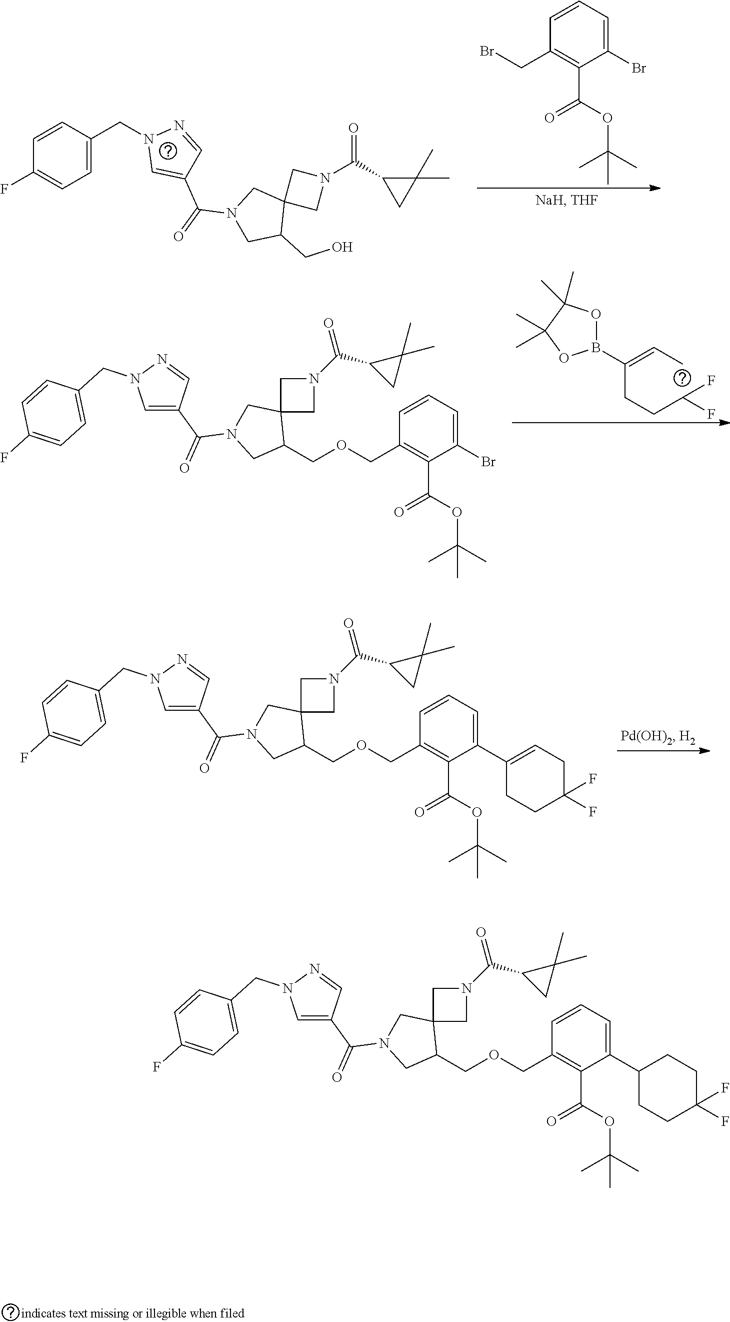

Step 1: tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate: To a solution of (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (260 mg, 0.59 mmol) and tert-butyl 2-bromo-6-(bromomethyl)benzoate (227 mg, 0.65 mmol) in THE (5 mL) at 0° C. was added sodium hydride (60 mg, 1.48 mmol), then the mixture was warmed to room temperature and stirred at room temperature for 2 h. The mixture was diluted with water (10 mL) and extracted with EtOAc (50 mL×3). The combined organic layers were dried over Na₂SO₄, filtered and concentrated. The mixture was purified by prep-TLC (DCM/MeOH=15/1) to afford tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (342.2 mg, 81.7%) as a yellow solid. LCMS m/z=709.1 [M+H]⁺; ¹H NMR (400 MHz, CDCl3) δ 7.97-7.77 (m, 2H), 7.55-7.46 (m, 1H), 7.35-7.27 (m, 1H), 7.26-7.16 (m, 3H), 7.09-6.98 (m, 2H), 5.28 (s, 2H), 4.65-4.46 (m, 2H), 4.37-4.04 (m, 2H), 3.98-3.75 (m, 4H), 3.64-3.51 (m, 2H), 3.49 (s, 2H), 2.60-2.47 (m, 1H), 1.61 (s, 9H), 1.22-1.16 (m, 1H), 1.15-1.10 (m, 6H), 1.01 (s, 1H), 0.77-0.68 (m, 1H).

Step 2: tert-butyl 3-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-4′,4′-difluoro-2′,3′,4′,5′-tetrahydro-[1,1′-biphenyl]-2-carboxylate: To a solution of tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (260 mg, 0.37 mmol) in dioxane/H₂O (9 mL/3 mL) were added 2-(4,4-difluorocyclohex-1-en-1-yl)-4,4,5,5-tetramethyl-1,3,2-dioxaborolane (107 mg, 0.44 mmol), K₃PO₄ (155 mg, 0.73 mmol) and Pd(PPh₃)₄ (42.32 mg, 0.037 mmol). The reaction mixture was stirred at 100° C. for 2 h then diluted with water (10 mL) and extracted with EtOAc (50 mL×3). The combined organic layers were washed with brine, dried over Na₂SO₄, filtered and concentrated to in vacuo. The residue was purified by afford by RP-column (78% CAN in water) to afford tert-butyl 3-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-4′,4′-difluoro-2′,3′,4′,5′-tetrahydro-[1,1′-biphenyl]-2-carboxylate (307 mg, 93.4%) as a white solid. LCMS m/z=747.6 [M+H]⁺; ¹H NMR (400 MHz, DMSO-d6) δ 8.32 (d, J=10.0 Hz, 1H), 7.81 (d, J=14.0 Hz, 1H), 7.32 (s, 4H), 7.24-7.13 (m, 3H), 5.37 (s, 1H), 5.35-5.31 (m, 2H), 4.52-4.46 (m, 2H), 4.06-4.00 (m, 2H), 3.96-3.90 (m, 1H), 3.70-3.56 (m, 7H), 2.68-2.55 (m, 3H), 2.19-2.05 (m, 2H), 2.03-1.94 (m, 2H), 1.47-1.41 (m, 10H), 1.11-1.01 (m, 5H), 0.96 (d, J=8.8 Hz, 1H), 0.87-0.82 (m, 1H), 0.71-0.60 (m, 1H).

Step 3: tert-butyl 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate: To a solution of tert-butyl 3-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-4′,4′-difluoro-2′,3′,4′,5′-tetrahydro-[1,1′-biphenyl]-2-carboxylate (280 mg, 0.37 mmol) in MeOH (5 mL) was added Pd(OH)₂ (112 mg, 0.79 mmol). The reaction mixture was stirred at 50° C. for 2 h with H₂ balloon. The mixture was filtrated with MeOH and concentrated under vacuum to afford crude tert-butyl 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (260 mg, 94%) which was used directly in the next step. LCMS m/z=749.5 [M+H]⁺; ¹H NMR (400 MHz, DMSO-d6) δ 8.33 (d, J=7.8 Hz, 1H), 7.81 (d, J=12.8 Hz, 1H), 7.36-7.23 (m, 5H), 7.22-7.12 (m, 2H), 5.34 (s, 2H), 4.52-4.42 (m, 2H), 4.26-4.11 (m, 1H), 4.08-3.34 (m, 10H), 2.73-2.64 (m, 1H), 2.20-2.07 (m, 2H), 1.92-1.64 (m, 6H), 1.58-1.47 (m, 9H), 1.39-1.28 (m, 1H), 1.10-0.95 (m, 6H), 0.86-0.82 (m, 1H), 0.70-0.61 (m, 111).

### 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid I′-80

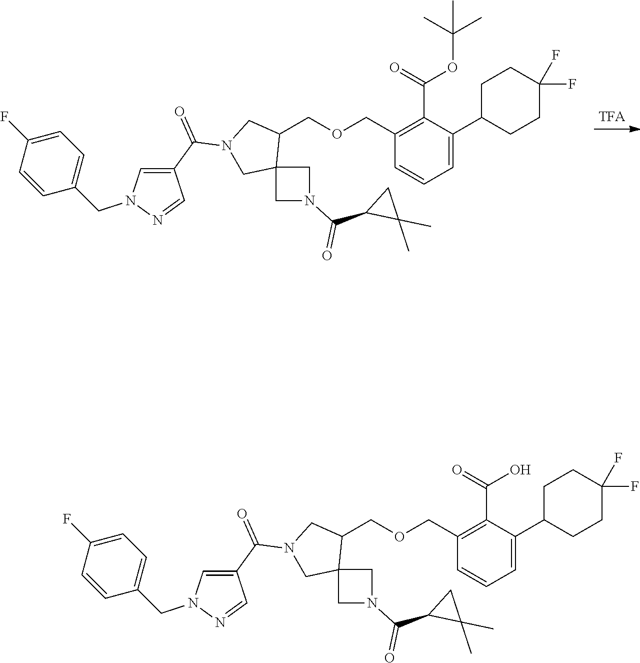 [SMILES: CC(C)(C)OC(=O)c1c(COCC2CN(C(=O)c3cnn(Cc4ccc(F)cc4)c3)CC23CN(C(=O)[C@H]2CC2(C)C)C3)cccc1C1CCC(F)(F)CC1 ; CC1(C)C[C@@H]1C(=O)N1CC2(CN(C(=O)c3cnn(Cc4ccc(F)cc4)c3)CC2COCc2cccc(C3CCC(F)(F)CC3)c2C(=O)O)C1]

To a solution of tert-butyl 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (50 mg, 0.066 mmol) in DCM (1 mL) was added TFA (0.5 ml). The reaction mixture was stirred at room temperature for 2 h. The mixture was purified by prep-TLC (DCM/MeOH=15/1, v/v) to afford 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (21 mg, 45%) as a white solid. LCMS m z=693.4 [M+H]⁺; ¹H NMR (400 MHz, DMSO-d6) δ 8.49-8.34 (m, 1H), 7.82 (d, J=12.0 Hz, 1H), 7.36-7.31 (m, 2H), 7.21-7.12 (m, 5H), 5.34 (s, 2H), 4.49 (s, 2H), 4.42-4.20 (m, 1H), 4.16-3.96 (m, 2H), 3.93-3.48 (m, 8H), 2.89-2.77 (m, 1H), 2.12-2.02 (m, 2H), 1.90-1.62 (m, 6H), 1.39-1.30 (m, 1H), 1.11-0.99 (m, 6H), 0.89-0.82 (m, 1H), 0.69-0.61 (m, 1H).

### 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylic acid I′-77

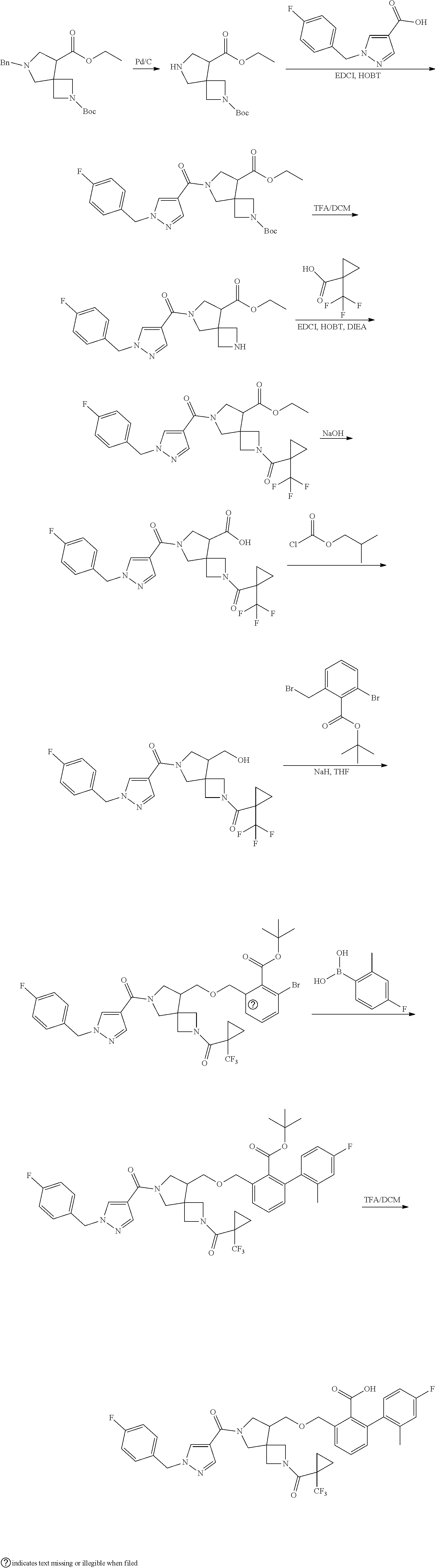

Step 1: 2-(tert-butyl)-8-ethyl-2,6-diazaspiro[3.4]octane-2,8-dicarboxylate: To a solution of 2-(tert-butyl) 8-ethyl 6-benzyl-2,6-diazaspiro[34]octane-2,8-dicarboxylate (10 g, 0.03 mol) in EtOAc (80 mL) was added EDC Pd/C (5 g). The reaction was stirred under a H₂ atmosphere at 50° C. for 2 days. The mixture was filtered through celite and concentrated to afford 2-(tert-butyl) 8-ethyl 2,6-diazaspiro[3.4]octane-2,8-dicarboxylate (6.8 g, 89.5%) as a white solid which was used directly in the next step without further purification. LCMS m/z=258.2 [M+H]⁺; ¹H NMR (400 z, Chloroform-d) δ 4.24-4.09 (m, 2H), 3.92 (dd, J=11.9, 8.8 Hz, 2H), 3.76 (dd, J=13.2, 8.8 Hz, 2H), 3.25-3.08 (m, 4H), 2.93 (dd, J=7.2, 5.6 Hz, 1H), 1.42 (s, 9H), 1.27 (t, J=7.2 Hz, 3H).

Step 2: 2-(tert-butyl)-8-ethyl 6-(1-(4-fluorobenzyl)-1H-pyrazole-4-carbonyl)-2,6-diazaspiro[3.4]octane-2,8-dicarboxylate: To a solution of 1-(4-fluorobenzyl)-1H-pyrazole-4-carboxylic acid (5.8 g, 0.03 mol) in DCM (50 mL) was added EDCI (6.9 g, 0.036 mol), HOBT (4.85 g, 0.036 mol) and DIEA (9.27 g, 0.072 mol). The mixture was stirred at room temperature for 30 min, 2-(tert-butyl)-8-ethyl-2,6-diazaspiro[3.4]octane-2,8-dicarboxylate (6.8 g, 0.024 mol) in DCM (20 mL) was added and the reaction was stirred at room temperature for another 2 h. The mixture was diluted with EtOAc (200 mL) and washed with water (200 mL×2), dried over Na₂SO₄, filtered and concentrated. The residue was purified by column chromatography on silica gel (eluent: Pet. Ether:EtOAc=5:1 to 1:1) to afford 2-(tert-butyl)-8-ethyl 6-(1-(4-fluorobenzyl)-1H-pyrazole-4-carbonyl)-2,6-diazaspiro[3.4]octane-2,8-dicarboxylate (8.1 g, 69.8%) as a yellow oil. LCMS m/z=487.2 [M+H]⁺; ¹H NMR (400 MHz, Chloroform-d) δ 7.86-7.75 (m, 2H), 7.25-7.17 (m, 2H), 7.02 (t, J=8.6 Hz, 2H), 5.25 (s, 2H), 4.31-4.13 (m, 2H), 4.02-3.86 (m, 5H), 3.85-3.72 (m, 3H), 3.21-3.06 (m, 1H), 1.41 (s, 9H), 1.27 (t, J=7.2 Hz, 3H).

Step 3: ethyl 6-(1-(4-fluorobenzyl)-1H-pyrazole-4-carbonyl)-2,6-diazaspiro[3.4]octane-8-carboxylate: To a solution of 2-(tert-butyl)-8-ethyl 6-(1-(4-fluorobenzyl)-1H-pyrazole-4-carbonyl)-2,6-diazaspiro[3.4]octane-2,8-dicarboxylate (14 g, 0.029 mol) in DCM (120 mL) was added TFA (40 mL). The mixture was stirred at room temperature for 2 h. The mixture was removed by distillation to afford ethyl 6-(1-(4-fluorobenzyl)-1H-pyrazole-4-carbonyl)-2,6-diazaspiro[3.4]octane-8-carboxylate (11.11 g, 99%) as a yellow oil which was used directly in the next step without further purification. LCMS m/z=387.1 [M+H]⁺.

Step 4: ethyl 6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylate: To a solution of 1-(trifluoromethyl)cyclopropane-1-carboxylic acid (3.14 g, 0.02 mol) in DCM (50 mL) was added EDCI (5.33 g, 0.028 mol), HOBT (3.76 g, 0.028 mol) and DIEA (7.18 g, 0.056 mol). The mixture was stirred at room temperature for 30 min, ethyl 6-(1-(4-fluorobenzyl)-1H-pyrazole-4-carbonyl)-2,6-diazaspiro[3.4]octane-8-carboxylate (7.16 g, 0.019 mol) in DCM (20 mL) was added and the reaction was stirred at room temperature for another 2 h. The mixture was diluted with EtOAc (200 mL) and washed with water (200 mL×2), dried over Na₂SO₄, filtered and concentrated. The residue was purified by column chromatography on silica gel (eluent: Pet. Ether:EtOAc=2:1 to 1:2) to afford ethyl 6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylate (8 g, 82.6%) as a colourless oil. LCMS m/z=523.2 [M+H]⁺; ¹H NMR (400 MHz, Chloroform-d) δ 7.86-7.75 (m, 2H), 7.26-7.19 (m, 2H), 7.04 (t, J=8.6 Hz, 2H), 5.27 (s, 2H), 4.37-4.16 (m, 4H), 4.10-3.70 (m, 6H), 3.26-3.09 (m, 1H), 1.28-1.25 (m, 4H), 1.22 (s, 3H).

Step 5: 6-(1-(4-fluorobenzyl)-1H-pyrazole-4-carbonyl)-2-(1-(trifluoromethyl) cyclopropane-1-carbonyl)-2,6-diazaspiro[3.4]octane-8-carboxylic acid: To a solution of ethyl 6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylate (7 g, 0.01 mol) in a mixture of THF:H₂O:EtOH (20 mL 5 mL: 5 mL) was added NaOH (1.4 g, 0.03 mol). The mixture was stirred at room temperature for 2 h. The mixture was diluted with water (100 mL) and washed with EtOAc (80 mL×2). The aqueous layer was collected and acidified with 1 M HCl (aq.) to pH-3 and extracted with EtOAc (80 mL×2). The combined organic layer was dried over Na₂SO₄, filtered and concentrated to afford 6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylic acid (4 g, 60.6%) as an off-yellow oil. LCMS m/z=493.1 [M−H]⁻; ¹H NMR (400 MHz, Chloroform-d) δ 7.91 (s, 1H), 7.82 (d, J=10.8 Hz, 1H), 7.26-7.20 (m, 2H), 7.03 (t, J=8.4 Hz, 2H), 5.28 (s, 2H), 4.71-4.10 (m, 3H), 4.11-3.73 (m, 5H), 3.27-3.10 (m, 1H), 1.20 (s, 4H).

Step 6: (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone: To a solution of 6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylic acid (4 g, 0.008 mol) in THE (30 mL) was added isobutyl carbonochloridate (1.5 g, 0.01 mol) and 4-methylmorpholine (1.1 g, 0.01 mol). The mixture was stirred at 0° C. for 30 min. NaBH₄ (0.9 g, 0.02 mol) in H₂O (8 mL) was added and the reaction was stirred at room temperature for another 2 h. The mixture was diluted with EtOAc (80 mL) and washed with water (60 mL×2), dried over Na₂SO₄, filtered and concentrated. The residue was purified by column chromatography on silica gel (eluent: Pet. Ether:EtOAc=2:1 to 1:2) to afford (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (2.4 g, 61.8%) as a white solid. LCMS m/z=481.2 [M+H]⁺; ¹H NMR (400 MHz, Chloroform-d) δ 7.82 (dd, J=21.0, 8.2 Hz, 2H), 7.25-7.19 (m, 2H), 7.04 (t, J=8.4 Hz, 2H), 5.27 (s, 2H), 4.58-3.42 (m, 10H), 2.58-2.35 (m, 1H), 1.21 (s, 4H).

Step 7: tert-butyl 2-bromo-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate: To a solution of (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (100 mg, 0.208 mmol) in dry THF (1 mL) was added tert-butyl 2-bromo-6-(bromomethyl)benzoate (87 mg, 0.25 mmol). The mixture was stirred at −78° C. for 30 min under N₂ atmosphere. NaH (30 mg, 1.25 mmol) was added at −78° C. under N₂ atmosphere and the reaction was stirred at room temperature for another 2 h under N₂ atmosphere. The mixture was diluted with EtOAc (10 mL) and washed with water (10 mL×2), dried over Na₂SO₄, filtered and concentrated to afford tert-butyl 2-bromo-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (150 mg, 96.7%) as an off-yellow oil. LCMS m/z=749.2 [M+H]⁺; ¹H NMR (400 MHz, Chloroform-d) δ 8.01-7.70 (m, 2H), 7.55-7.47 (m, 1H), 7.26-7.16 (m, 4H), 7.10-6.95 (m, 2H), 5.28 (s, 2H), 4.61-4.48 (m, 2H), 4.49-4.16 (m, 2H), 4.07-3.40 (m, 8H), 2.55 (d, J=8.4 Hz, 1H), 1.64-1.62 (m, 4H), 1.59 (s, 9H).

Step 8: tert-butyl 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylate: To a solution of tert-butyl 2-bromo-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (100 mg, 0.13 mmol) in a mixture of 1,4-dioxane:H₂O (2 mL:0.6 mL) was added (4-fluoro-2-methylphenyl)boronic acid (22 mg, 0.15 mmol), Pd(PPh₃)₄ (15.4 mg, 0.01 mmol) and K₂CO₃ (37 mg, 0.27 mmol). The mixture was stirred at 100° C. for 2 h under N₂ atmosphere. The mixture was diluted with EtOAc (10 mL) and washed with water (10 mL×2), dried over Na₂SO₄, filtered and concentrated. The residue was purified by pre-TLC to afford tert-butyl 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylate (90 mg, 86.5%) as a white solid. LCMS m/z=779.3 [M+H]⁺; ¹H NMR (400 MHz, Chloroform-d) δ 7.83 (t, J=24.3 Hz, 2H), 7.37 (d, J=6.7 Hz, 2H), 7.25-7.16 (m, 2H), 7.15-6.98 (m, 4H), 6.98-6.81 (m, 2H), 5.27 (d, J=10.5 Hz, 2H), 4.68-4.55 (m, 2H), 4.43-3.75 (m, 7H), 3.69-3.50 (m, 3H), 2.58 (d, J=20.9 Hz, 1H), 2.08 (d, J=3.6 Hz, 3H), 1.26-1.22 (m, 2H), 1.20-1.17 (m, 2H), 1.16 (s, 9H).

Step 9: 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylic acid: To a solution of tert-butyl 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylate (90 mg, 0.12 mmol) in DCM (4 mL) was added TFA (1 mL). The mixture was stirred at room temperature for 2 h. The mixture was concentrated in vacuo. The residue was purified by pre-TLC (DCM:MeOH=10:1) to afford 4′-fluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′-methyl-[1,1′-biphenyl]-2-carboxylic acid (60 mg, 72.2N) as a white solid. LCMS m/z=721.3 [M−H]⁻; ¹H NMR (400 MHz, Chloroform-d) δ 7.84 (s, 1H), 7.76 (s, 1H), 7.40-7.37 (m, 1H), 7.28 (s, 1H), 7.21 (s, 2H), 7.15 (d, J=7.5 Hz, 1H), 7.09-6.98 (d, 3H), 6.90 (d, 1H), 6.82-6.80 (m, 1H), 5.22 (s, 2H), 4.83-4.46 (m, 2H), 4.39 (s, 1H), 4.28-4.16 (m, 1H), 4.05 (s, 1H), 3.93-3.53 (m, 7H), 2.50 (s, 1H), 2.09 (s, 3H), 1.255-1.19 (i, 2H), 1.19-1.16 (d, 2H).

TABLE 12
The compounds listed in Table 12 were synthesized from tert-butyl 2-bromo-
6-(((6-(1-(4-fluorobenzyl)-1H-pyrazole-4-carbonyl)-2-(1-(trifluoromethyl)cyclopropane-1-
carbonyl)-2,6-diazaspiro[3.4]octan-8-yl)methoxy)methyl)benzoate according to the procedures
outlined for I′-77 using the appropriate commercially available reagents and/or intermediates
described elsewhere.

| Cmpd. | | | |
|---|---|---|---|
| No. | Compound | ¹HNMR | LCMS |
| I′-73 | 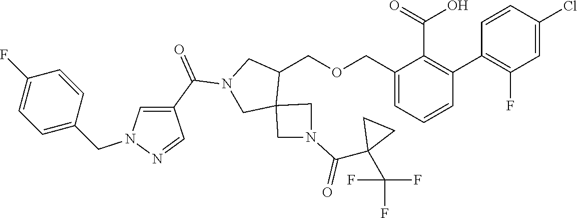 [SMILES: O=C(O)c1c(COCC2CN(C(=O)c3cnn(Cc4ccc(F)cc4)c3)CC23CN(C(=O)C2(C(F)(F)F)CC2)C3)cccc1-c1ccc(Cl)cc1F] | ¹H NMR (400 MHz, DMSO-d₆) δ 13.39 − 12.71 (m, 1H), 8.33 (s, 1H), 7.81 (d, J = 15.2 Hz, 1H), 7.53 − 7.41 (m, 3H), 7.38 − 7.25 (m, 5H), 7.22 − 7.10 (m, 2H), 5.31 (d, J = 24.8 Hz, 2H), 4.63 (s, 2H), 4.48 − 3.89 (m, 4H), 3.84 − 3.38 (m, 6H), 2.68 − 2.52 (m, 1H), 1.26 − 1.08 (m, 4H). | m/z = 743.3 [M + H]⁺ |
| I′-67 | 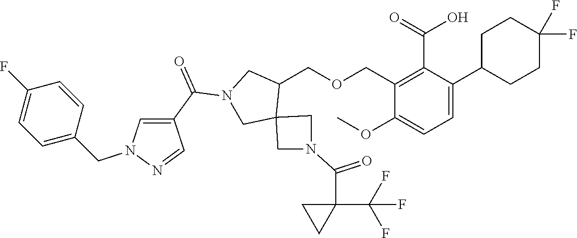 [SMILES: COc1ccc(C2CCC(F)(F)CC2)c(C(=O)O)c1COCC1CN(C(=O)c2cnn(Cc3ccc(F)cc3)c2)CC12CN(C(=O)C1(C(F)(F)F)CC1)C2] | ¹H NMR (400 MHz, CD₃OD) δ 8.21 (d, J = 30.8 Hz, 1H), 7.93 − 7.86 (m, 1H), 7.39 − 7.23 (m, 3H), 7.15 − 6.93 (m, 3H), 5.35 (s, 2H), 4.65 − 4.57 (m, 2H), 4.30 − 3.49 (m, 13H), 2.79 − 2.52 (m, 2H), 2.18 − 2.04 (m, 2H), 1.93 − 1.70 (m, 6H), 1.20 − 1.03 (m, 4H). | m/z = 763.4 [M + H]⁺ |

### 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile I′-75

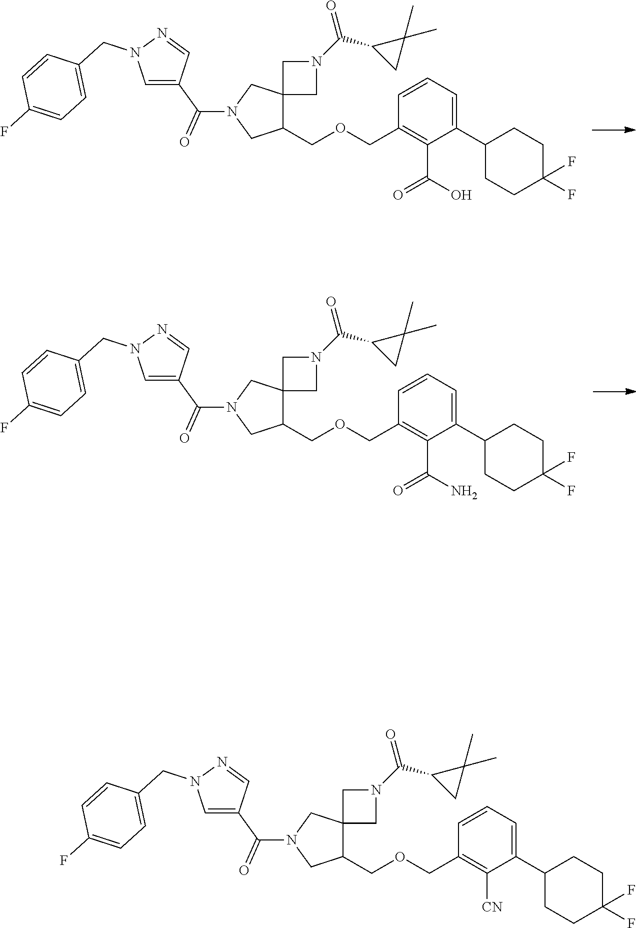 [SMILES: CC1(C)C[C@@H]1C(=O)N1CC2(CN(C(=O)c3cnn(Cc4ccc(F)cc4)c3)CC2COCc2cccc(C3CCC(F)(F)CC3)c2C(=O)O)C1 ; CC1(C)C[C@@H]1C(=O)N1CC2(CN(C(=O)c3cnn(Cc4ccc(F)cc4)c3)CC2COCc2cccc(C3CCC(F)(F)CC3)c2C(N)=O)C1 ; CC1(C)C[C@@H]1C(=O)N1CC2(CN(C(=O)c3cnn(Cc4ccc(F)cc4)c3)CC2COCc2cccc(C3CCC(F)(F)CC3)c2C#N)C1]

Step 1: 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide: To a solution of 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (126 mg, 0.18 mmol) in DMF (2.5 mL) was added HATU (69.2 mg, 0.18 mmol) and DIPEA (70.5 mg, 0.55 mmol). The resulting mixture was stirred at room temperature for 2 h. The crude residue obtained was purified by prep-TLC (DCM/MeOH=15/1) to afford 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (118 mg, 84%) as a white solid. ¹H NMR (400 MHz, DMSO-d6) δ 8.36 (d, J=11.6 Hz, 1H), 7.85-7.79 (m, 2H), 7.54 (s, 1H), 7.36-7.31 (m, 2H), 7.23 (s, 3H), 7.19-7.15 (m, 2H), 5.34 (s, 2H), 4.50 (s, 2H), 4.07-3.50 (m, 10H), 2.80-2.73 (m, 1H), 2.10 (s, 2H), 2.03-1.95 (m, 4H), 1.89-1.85 (m, 2H), 1.70-1.65 (m, 1H), 1.48-1.43 (m, 1H), 1.10 (s, 3H), 1.06-1.02 (m, 311), 0.86-0.85 (m, 1H), 0.67 (s, 1H).

Step 2: 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile: To a solution of 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (118 mg, 0.17 mmol) in DCM (8 mL) was added N,N-Diethylethanamin (51.78 mg, 0.51 mmol) and Trifluoroacetic anhydride (53.74 mg, 0.26 mmol). The reaction mixture was stirred at room temperature for 0.5 h. The mixture was purified by prep-TLC (DCM/MeOH=20/1) to afford 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile (80 mg, 70%) as a white solid. LCMS m/z=674.4 [M+H]⁺; ¹H NMR (400 MHz, DMSO-d6) δ 8.36-8.29 (m, 1H), 7.82 (s, 1H), 7.64-7.56 (m, 1H), 7.50-7.40 (m, 2H), 7.37-7.30 (m, 2H), 7.23-7.13 (m, 2H), 5.34 (s, 2H), 4.66 (s, 2H), 4.46-3.54 (m, 10H), 3.44-3.39 (m, 1H), 3.11-2.98 (m, 1H), 2.18-2.09 (m, 2H), 2.04-1.83 (m, 4H), 1.82-1.69 (m, 2H), 1.39-1.29 (m, 1H), 1.11-0.97 (m, 6H), 0.85 (s, 1H), 0.66 (s, 1H).

### 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid I-113

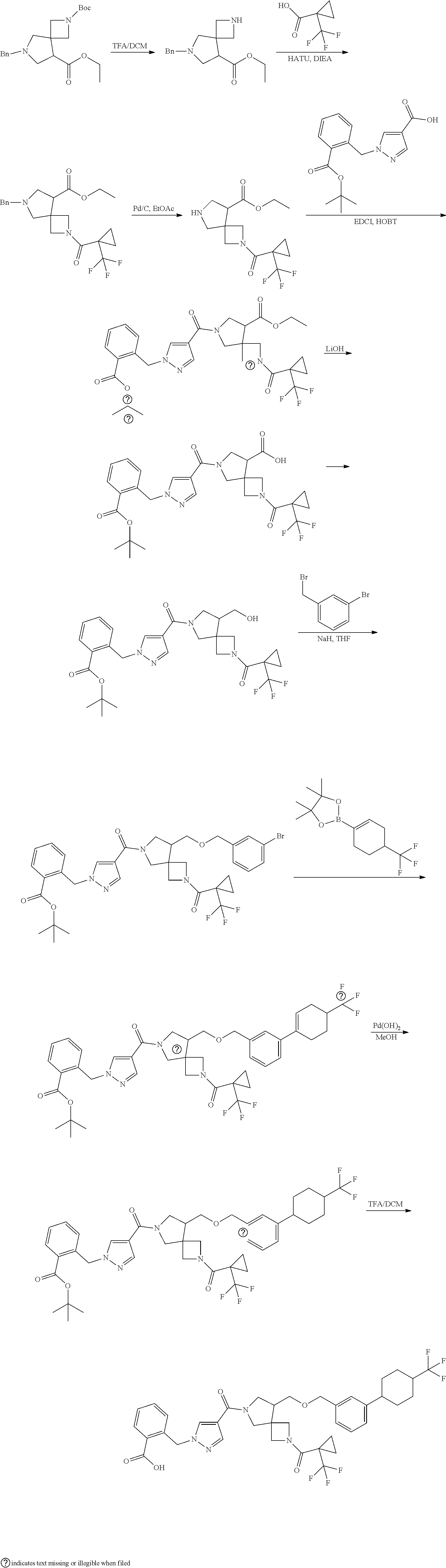

Step 1: ethyl 6-benzyl-2,6-diazaspiro[3.4]octane-8-carboxylate To a solution of 2-(tert-butyl) 8-ethyl 6-benzyl-2,6-diazaspiro[3.4]octane-2,8-dicarboxylate (10 g, 26.7 mmol) in DCM (50 mL) was added TFA (20 mL). The reaction mixture was stirred at room temperature for 2 h. The mixture was concentrated in vacuo to afford ethyl 6-benzyl-2,6-diazaspiro[3.4]octane-8-carboxylate (7.3 g, 100%) as a yellow oil. LCMS m/z=275.3 [M+H]⁺.

Step 2: ethyl 6-benzyl-2-(1-(trifluoromethyl)cyclopropane-1-carbonyl)-2,6-diazaspiro[3.4]octane-8-carboxylate To a solution of 1-(trifluoromethyl)cyclopropane-1-carboxylic acid (410 mg, 2.67 mmol) in DCM (15 mL) was added HATU (1 g, 2.67 mmol) and the mixture stirred at room temperature for 30 min, ethyl 6-benzyl-2,6-diazaspiro[3.4]octane-8-carboxylate (730 mg, 2.67 mmol) and DIPEA (1.38 g, 10.7 mmol) were added and the reaction stirred at room temperature for another 2 h. The mixture was diluted with water (50 mL), extracted with DCM (30 mL×2) and the combined organic layers washed with brine, dried over Na₂SO₄ and filtered. The solvent was removed and the residue purified by column chromatography on silica gel (eluent: DCM:MeOH=80:1) to afford the ethyl 6-benzyl-2-(1-(trifluoromethyl)cyclopropane-1-carbonyl)-2,6-diazaspiro[3.4]octane-8-carboxylate (840 mg, 77%) as a white solid. LCMS m/z=411.3 [M+H]⁺; ¹H NMR (400 MHz, DMSO-d₆) δ 7.41-7.39 (m, 5H), 4.33-3.49 (m, 11H), 3.23-3.08 (m, 2H), 1.28-1.23 (m, 3H), 1.20-1.17 (m, 4H).

Step 3: ethyl 2-(1-(trifluoromethyl)cyclopropane-1-carbonyl)-2,6-diazaspiro[3.4]octane-8-carboxylate To a solution of ethyl 6-benzyl-2-(1-(trifluoromethyl)cyclopropane-1-carbonyl)-2,6-diazaspiro[3.4]octane-8-carboxylate (1.38 g, 3.36 mmol) in EtOAc (12 mL) was added Pd/C (554 mg). The reaction mixture was stirred at 50° C. overnight with H₂ balloon. The mixture was filtrated with MeOH and concentrated under vacuum to afford crude ethyl 2-(1-(trifluoromethyl)cyclopropane-1-carbonyl)-2,6-diazaspiro[3.4]octane-8-carboxylate (1 g, 100%) which was used directly in the next step. LCMS m/z=321.2 [M+H]⁺.

Step 4: ethyl 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylate To a solution of 1-(2-(tert-butoxycarbonyl)benzyl)-1H-pyrazole-4-carboxylic acid (3.1 g, 10.3 mmol) in DMF (30 mL) were added ethyl 2-(1-(trifluoromethyl)cyclopropane-1-carbonyl)-2,6-diazaspiro[3.4]octane-8-carboxylate (3.3 g, 10.3 mmol), EDCI (2.96 g, 15.45 mmol), HOBT (2.1 g, 15.45 mmol) and DIPEA (5.3 g, 41.21 mmol). The reaction mixture was purified by RP-column (60% ACN in water) to afford ethyl 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylate (3.15 g, 50.8%) as a yellow oil. LCMS m/z=605.4 [M+H]⁺; ¹H NMR (400 MHz, DMSO-d₆) δ 8.27 (d, J=16.8 Hz, 1H), 7.91-7.83 (m, 2H), 7.54-7.40 (m, 2H), 6.80 (d, J=7.8 Hz, 1H), 5.70 (s, 2H), 4.41-4.16 (m, 2H), 4.14-3.68 (m, 8H), 3.49-3.40 (m, 1H), 1.56-1.53 (m, 9H), 1.26-1.15 (m, 7H).

Step 5: 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid To a solution of ethyl 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylate (3.15 g, 5.21 mmol) in a mixture of THF/EtOH/H₂O (20 mL/5 mL/5 mL) at 0° C. was added a solution of lithium hydroxide monohydrate (312 mg, 13.02 mmol) in H₂O (1 mL). The reaction mixture was stirred at room temperature for 2 h then diluted with water (10 mL) and extracted with EtOAc (20 mL). The aqueous layer was collected and acidified with 1 M HCl to pH˜2 and extracted with EtOAc (50 mL×3). The combined organic layers were washed with brine, dried over Na₂SO₄, filtered and concentrated to afford crude 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid (2.6 g, 86.7%) as a yellow oil which was used directly in the next step. LCMS m/z=577.3 [M+H]⁺; ¹H NMR (400 MHz, DMSO-d₆) δ 8.28 (d, J=15.2 Hz, 1H), 7.90-7.82 (m, 2H), 7.54-7.40 (m, 2H), 6.79 (d, J=7.6 Hz, 1H), 5.70 (s, 2H), 4.40-4.19 (m, 2H), 4.05-3.98 (m, 2H), 3.93-3.69 (m, 4H), 3.66 (d, J=6.8 Hz, 1H), 1.55 (s, 9H), 1.20-1.14 (m, 4H).

Step 6: tert-butyl 2-((4-(8-(hydroxymethyl)-2-(1-(trifluoromethyl)cyclopropane-1-carbonyl)-2,6-diazaspiro[3.4]octane-6-carbonyl)-1H-pyrazol-1-yl)methyl)benzoate To a solution of 6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid (2.78 g, 4.82 mmol) in THE (20 mL) at 0° C. was added a solution of isobutyl carbonochloridate (948 mg, 6.94 mmol) and 4-methylmorpholine (654 mg, 6.46 mmol). The reaction mixture was stirred at 0° C. for 0.5 h. Then, NaBH₄ (547 mg, 14.5 mmol) in H₂O (10 mL) was added to the mixture. The reaction mixture was stirred at 0° C. for 2 h then diluted with water (10 mL) and extracted with EtOAc (30 mL×3). The combined organic layers were washed with brine, dried over Na₂SO₄, filtered and concentrated to in vacuo. The residue was purified by afford by column chromatography on silica gel (eluent: DCM:MeOH=20:1) to afford the tert-butyl 2-((4-(8-(hydroxymethyl)-2-(1-(trifluoromethyl)cyclopropane-1-carbonyl)-2,6-diazaspiro[3.4]octane-6-carbonyl)-1H-pyrazol-1-yl)methyl)benzoate (2 g, 74%) as a white solid. LCMS m/z=563.3 [M+H]⁺; ¹H NMR (400 MHz, DMSO-d₆) δ 8.26 (d, J=6.0 Hz, 1H), 7.88-7.83 (m, 2H), 7.54-7.40 (m, 2H), 6.79 (t, J=6.6 Hz, 1H), 5.70 (s, 2H), 4.79 (d, J=4.8 Hz, 1H), 4.31-4.06 (m, 2H), 3.95-3.66 (m, 4H), 3.63-3.35 (m, 4H), 2.44-2.31 (m, 1H), 1.55 (s, 9H), 1.15 (s, 4H).

Step 7: tert-butyl 2-((4-(8-(((3-bromobenzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(hydroxymethyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 0.36 mmol) in dry THF (3 mL) at 0° C. were added 1-bromo-3-(bromomethyl)benzene (106 mg, 0.43 mmol) and NaH (22 mg, 0.89 mmol). The reaction mixture was stirred at room temperature overnight then diluted with water (10 mL) and extracted with EtOAc (10 mL×3). The combined organic layers were washed with brine, dried over Na₂SO₄, filtered and concentrated in vacuo. The residue was purified by prep-TLC (eluent: DCM:MeOH=20:1) to afford the tert-butyl 2-((4-(8-(((3-bromobenzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (190 mg, 73%) as a white solid. LCMS w % z=731.3 [M+H]⁺; ¹H NMR (400 MHz, DMSO-d₆) δ 8.25 (d, J=12.0 Hz, 1H), 7.88-7.83 (m, 2H), 7.52-7.40 (m, 4H), 7.29 (d, J=7.0 Hz, 2H), 6.80 (d, J=7.8 Hz, 1H), 5.70 (s, 2H), 4.50-4.47 (m, 2H), 4.31-4.06 (m, 2H), 3.98-3.50 (m, 8H), 2.69-2.56 (m, 1H), 1.56-1.53 (m, 9H), 1.13 (s, 4H).

Step 8: tert-butyl 2-((4-(8-(((4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((3-bromobenzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (140 mg, 0.19 mmol) in dioxane/H₂O (3 mL/1 mL) were added 4,4,5,5-tetramethyl-2-(4-(trifluoromethyl)cyclohex-1-en-1-yl)-1,3,2-dioxaborolane (58 mg, 0.21 mmol), K₃PO₄ (122 mg, 0.57 mmol) and Pd(PPh₃)₄ (22 mg, 0.02 mmol). The reaction mixture was stirred at 100° C. for 2 h then diluted with water (10 mL) and extracted with EtOAc (10 mL×3). The combined organic layers were washed with brine, dried over Na₂SO₄, filtered and concentrated in vacuo. The residue was purified by RP-column (78% CAN in water) to afford the tert-butyl 2-((4-(8-(((4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (65 mg, 42%) as a white solid. LCMS m/z=747.4 [M+H−56]⁺; ¹H NMR (400 MHz, DMSO-d₆) δ 8.25 (d, J=9.0 Hz, 1H), 7.85 (d, J=10.0 Hz, 2H), 7.53-7.47 (m, 1H), 7.45-7.40 (m, 1H), 7.35-7.24 (m, 3H), 7.20-7.14 (m, 1H), 6.78 (t, J=8.0 Hz, 1H), 6.12 (s, 1H), 5.69 (s, 2H), 4.50-4.46 (m, 2H), 4.06-3.32 (m, 11H), 2.48-1.95 (m, 7H), 1.55 (s, 9H), 1.14 (s, 4H).

Step 9: tert-butyl 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (55 mg, 0.07 mmol) in MeOH (1 mL) was added Pd(OH)₂ (24 mg, 0.17 mmol). The reaction mixture was stirred at 50° C. for 2 h with H₂ balloon. The mixture was filtrated with MeOH and concentrated under vacuum to afford crude tert-butyl 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (55 mg, 100%) which was used directly in the next step. LCMS m/z=803.4 [M+H]⁺.

Step 10: 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid I-113 To a solution of tert-butyl 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (55 mg, 0.07 mmol) in DCM (1 mL) was added TFA (0.5 mL). The reaction mixture was stirred at room temperature for 2 h. The mixture was purified by prep-TLC (eluent: DCM:MeOH=10:1) to afford 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid (I-113, 75 mg, 68%) as a white solid. LCMS m/z=747.4 [M+H]⁺; ¹H NMR (400 MHz, DMSO-d₆) δ 8.30 (s, 1H), 7.93-7.81 (m, 2H), 7.47-7.36 (m, 2H), 7.26-7.08 (m, 4H), 6.76 (d, =7.6 Hz, 1H), 5.75 (s, 2H), 4.47 (s, 2H), 4.13-3.51 (m, 12H), 1.96-1.32 (m, 9H), 1.12 (s, 4H).

### 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid I-115

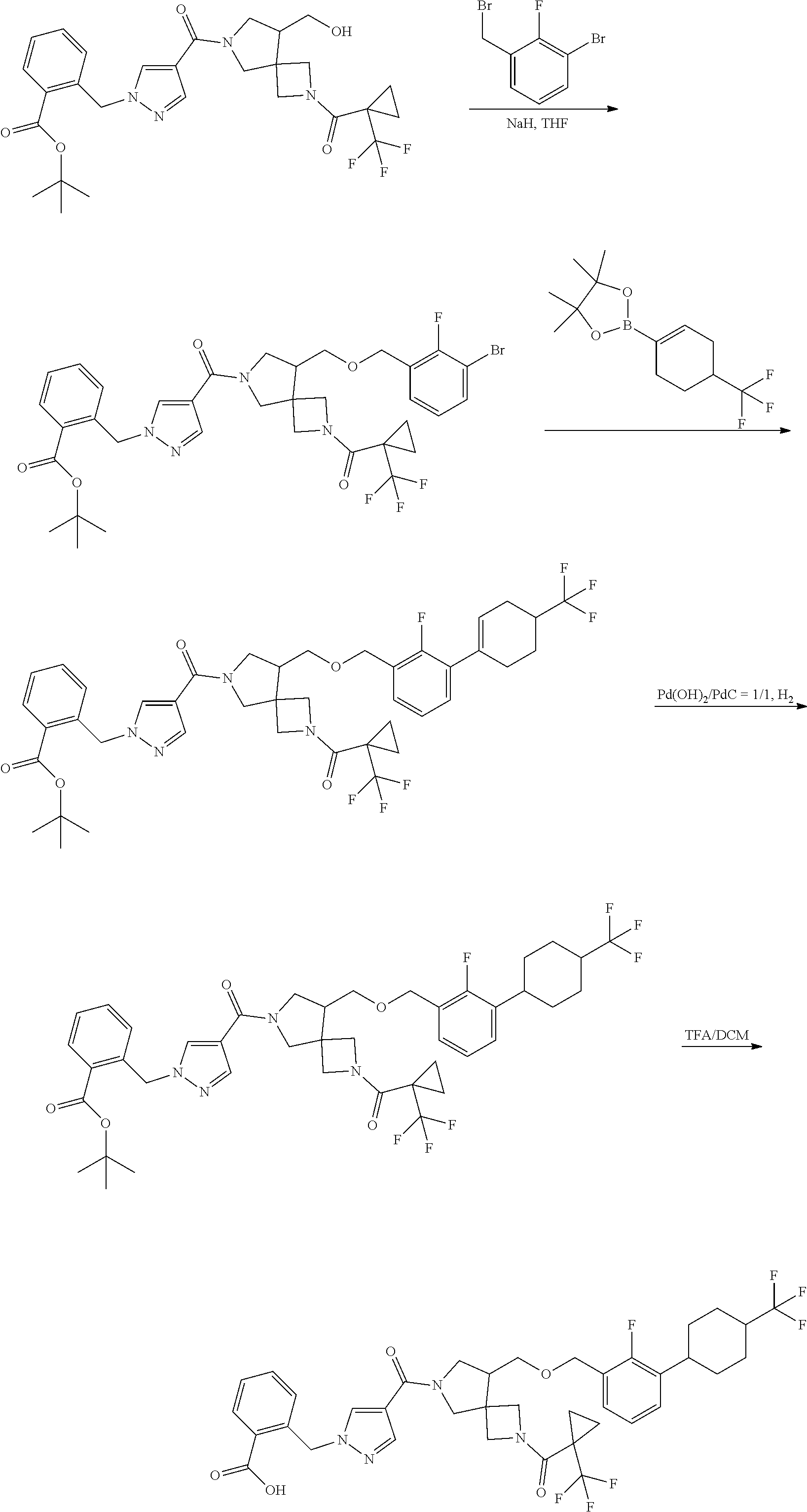

Step 1: tert-butyl 2-((4-(8-(((3-bromo-2-fluorobenzyl)oxy) methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(hydroxymethyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 0.36 mmol) in THF (4 mL) was added 1-bromo-3-(bromomethyl)-2-fluorobenzene (95 mg, 0.36 mmol) and NaH (71 mg, 1.78 mmol) at 0° C., the reaction mixture was stirred at room temperature overnight. The reaction was quenched with water (50 mL) and extracted with EtOAc (20 mL×3). The combined organic layers were washed with brine, dried over Na₂SO₄, filtered, concentrated, and purified by prep-TLC (eluent: DCM:MeOH=20:1) to afford tert-butyl 2-((4-(8-(((3-bromo-2-fluorobenzyl)oxy) methyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 75%) as a colorless oil. LCMS m/z=749.8 [M+H]⁺; ¹H NMR (400 MHz, DMSO-d₆) δ 8.25 (d, J=14.4 Hz, 1H), 7.85 (d, J=12.6 Hz, 2H), 7.64 (t, J=7.4 Hz, 1H), 7.54-7.47 (m, 1H), 7.41 (q, J=7.4 Hz, 2H), 7.16-7.09 (m, 1H), 6.79 (d, J=7.8 Hz, 1H), 5.71-5.68 (m, 2H), 4.57 (d, J=5.2 Hz, 2H), 4.45-4.07 (m, 2H), 4.02-3.87 (m, 2H), 3.87-3.49 (m, 6H), 2.00 (q, J=7.2, 6.8 Hz, 1H), 1.54 (d, J=4.2 Hz, 9H), 1.24-1.23 (m, 2H), 1.14-1.12 (m, 2H).

Step 2: tert-butyl 2-((4-(8-(((2-fluoro-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((3-bromo-2-fluorobenzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 0.27 mmol) in dioxane (4 mL) and H₂O (1 mL) was added 4,4,5,5-tetramethyl-2-(4-(trifluoromethyl)cyclohex-1-en-1-yl)-1,3,2-dioxaborolane (81 mg, 0.29 mmol), Pd(PPh₃)₄ (31 mg, 0.03 mmol) and K₃PO₄ (113 mg, 0.53 mmol), the reaction mixture was stirred at 100° C. for 2 h. The reaction was quenched with water (50 mL) and extracted with EtOAc (20 mL×3). The combined organic layers were washed with brine, dried over Na₂SO₄, filtered, concentrated, and purified by prep-TLC (eluent: DCM:MeOH=20:1) to afford tert-butyl 2-((4-(8-(((2-fluoro-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (165 mg, 75%) as a yellow oil. LCMS m/z=819.3 [M+H]⁺; ¹H NMR (400 MHz, DMSO-d₆) δ 8.25 (d, J=11.4 Hz, 1H), 7.88-7.82 (m, 2H), 7.64-7.59 (m, 2H), 7.25 (q, J=8.2, 7.6 Hz, 2H), 7.14-7.08 (m, 1H), 6.78 (t, J=8.4 Hz, 1H), 5.92-5.86 (m, 1H), 5.72-5.67 (m, 2H), 5.32 (t, J=4.8 Hz, 1H), 4.55-4.51 (m, 2H), 3.96-3.92 (m, 1H), 3.85-3.48 (m, 7H), 2.68-2.54 (m, 3H), 2.42-2.11 (m, 3H), 2.00 (q, J=6.8, 6.4 Hz, 3H), 1.54 (d, J=3.8 Hz, 9H), 1.24-1.23 (m, 4H).

Step 3: tert-butyl 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((2-fluoro-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (110 mg, 0.13 mmol) in MeOH (6 mL) was added Pd(OH)₂ (44 mg) and Pd/C (44 mg), the reaction mixture was stirred under a H₂ atmosphere at 50° C. overnight. The mixture was filtered and concentrated to afford tert-butyl 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (100 mg, 90%) which was used directly in the next step. LCMS m/z=821.3 [M+H]⁺; ¹H NMR (400 MHz, DMSO-d₆) δ 8.25 (d, J=11.0 Hz, 1H), 7.87-7.82 (m, 2H), 7.65-7.59 (m, 1H), 7.42 (t, J=7.6 Hz, 1H), 7.27-7.17 (m, 2H), 7.13-7.07 (m, 1H), 6.79 (d, J=7.8 Hz, 1H), 5.70 (d, J=2.4 Hz, 2H), 4.55-4.50 (m, 2H), 4.30-4.07 (m, 2H), 3.96-3.50 (m, 8H), 2.98-2.53 (m, 3H), 1.98-1.82 (m, 3H), 1.75-1.68 (m, 3H), 1.55 (d, J=3.8 Hz, 9H), 1.48-1.27 (m, 2H), 1.25-1.22 (m, 4H).

Step 4: 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid I-115 To a solution of tert-butyl 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (100 mg, 0.12 mmol) in DCM (4 mL) was added TFA (2 mL). The reaction mixture was stirred at room temperature for 1 h. The solvent was removed under vacuum, purified by prep-HPLC to afford 2-((4-(8-(((2-fluoro-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid (I-115, 35 mg, 37%) as a white solid. LCMS m/z=765.7 [M+H]⁺; ¹H NMR (400 MHz, DMSO-d₆) δ 8.29 (d, J=7.8 Hz, 1H), 7.95-7.82 (m, 2H), 7.51-7.38 (m, 2H), 7.22 (q, J=10.0, 8.6 Hz, 2H), 7.14-7.06 (m, 1H), 6.76 (d, J=7.6 Hz, 1H), 5.75 (s, 2H), 4.52 (d, J=4.6 Hz, 2H), 4.23-3.51 (m, 10H), 2.98-2.54 (m, 3H), 1.97-1.38 (m, 8H), 1.22-1.06 (m, 4H).

### 2-((4-(8-(((2-(difluoromethoxy)-3-(4-(trifluoromethyl)cyclohexyl)benzyl)oxy) methyl)-2-(1-(trifluoromethyl)cyclopropane-1-carbonyl)-2,6-diazaspiro[3.4]octane-6-carbonyl)-1H-pyrazol-1-yl)methyl)benzoic acid I′-74

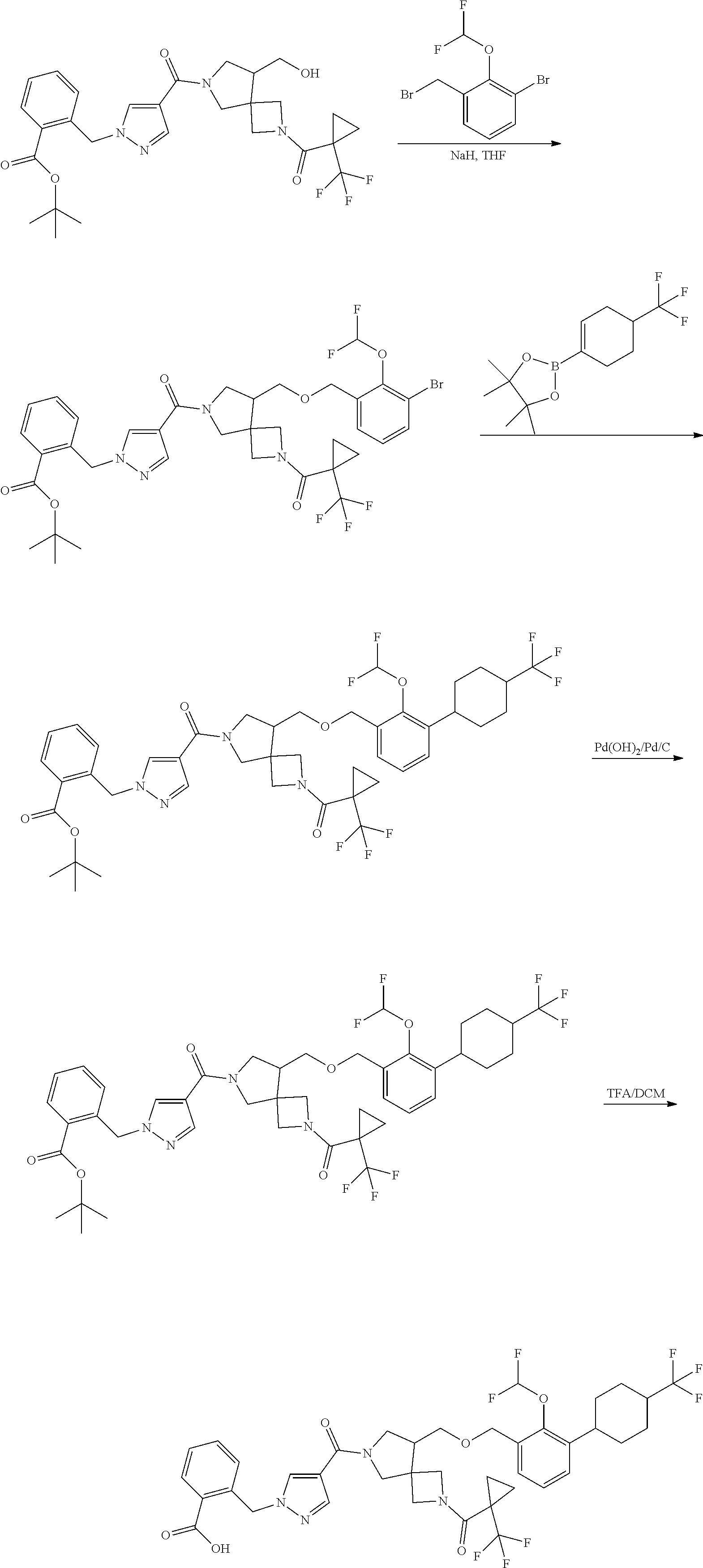

Step 1: tert-butyl 2-((4-(8-(((3-bromo-2-(difluoromethoxy)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of 1-bromo-3-(bromomethyl)-2-(difluoromethoxy)benzene (100 mg, 0.32 mmol) in THF (4 mL) was added tert-butyl 2-((4-(8-(hydroxymethyl)-2-(1-(trifluoromethyl)cyclopropane-1-carbonyl)-2,6-diazaspiro[3.4]octane-6-carbonyl)-1H-pyrazol-1-yl)methyl)benzoate (179 mg, 0.32 mmol) and NaH (23 mg, 0.96 mmol). The reaction was stirred at room temperature for overnight. The mixture was diluted with water (40 mL) and extracted with EtOAc (50 mL×2). The combined organic layers were washed with brine, dried over Na₂SO₄, filtered and concentrated. The residue obtained was purified by prep-TLC (DCM:MeOH=20:1) to afford tert-butyl 2-((4-(8-(((3-bromo-2-(difluoromethoxy)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 79.2%) as a colorless oil. LCMS m/z=797.2 [M+H]⁺; ¹H NMR (DMSO-d₆, 400 MHz) δ 8.25 (d, J=13.4 Hz, 1H), 7.85 (d, J=11.6 Hz, 2H), 7.69 (dd, J=8.0 Hz, 1H), 7.54-7.41 (m, 3H), 7.25-7.18 (m, 1H), 6.80 (d, J=7.4 Hz, 1H), 5.70 (s, 2H), 4.57 (d, J=6.4 Hz, 2H), 4.13 (s, 1H), 4.00-3.51 (m, 8H), 3.41-3.34 (m, 1H), 1.99 (t, J=7.2 Hz, 1H), 1.54 (d, J=4.6 Hz, 9H), 1.24 (s, 3H), 1.13 (s, 2H).

Step 2: tert-butyl 2-((4-(8-(((2-(difluoromethoxy)-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((3-bromo-2-(difluoromethoxy)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (200 mg, 0.25 mmol) in dioxane/water (4 mL, 3:1) under a N₂ atmosphere was added 4,4,5,5-tetramethyl-2-(4-(trifluoromethyl)cyclohex-1-en-1-yl)-1,3,2-dioxaborolane (90 mg, 0.33 mmol), Pd(PPh₃)₄ (29 mg, 0.03 mmol) and K₃PO₄ (107 mg, 0.50 mmol) and the mixture was heated at 100° C. for 2 h. The mixture was diluted with water (50 mL) and extracted with EtOAc (60 mL×2). The combined organic layers were washed with brine, dried over Na₂SO₄, filtered and concentrated. The residue obtained was purified by prep-TLC (DCM:MeOH=20:1) to afford tert-butyl 2-((4-(8-(((2-(difluoromethoxy)-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (175 mg, 80.1%) as a yellow oil. LCMS m/z=867.3 [M+H]⁺; ¹H NMR (DMSO-d₆, 400 MHz) δ 8.25 (d, J=10.4 Hz, 1H), 7.85 (d, J=11.2 Hz, 2H), 7.64-7.42 (m, 3H), 7.34 (t, J=7.8 Hz, 1H), 7.26-7.17 (m, 2H), 6.81-6.74 (m, 1H), 5.81-5.75 (m, 1H), 5.69 (d, J=4.4 Hz, 2H), 4.53 (d, J=6.6 Hz, 2H), 3.95 (s, 1H), 3.83 (t, J=8.8 Hz, 1H), 3.74-3.64 (m, 2H), 3.63-3.51 (m, 2H), 3.42-3.34 (m, 2H), 3.17 (s, 4H), 2.43-2.31 (m, 3H), 2.16 (t, J=14.6 Hz, 1H), 2.04-1.97 (m, 2H), 1.54 (d, J=4.2 Hz, 9H), 1.24 (s, 2H), 1.13 (s, 2H)

Step 3: tert-butyl 2-((4-(8-(((2-(difluoromethoxy)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of tert-butyl 2-((4-(8-(((2-(difluoromethoxy)-4′-(trifluoromethyl)-2′,3′,4′,5′-tetrahydro-[1,1′-biphenyl]-3-yl)methoxy)methyl)-2-(1-(trifluoromethyl)cyclopropane-1-carbonyl)-2,6-diazaspiro[3.4]octane-6-carbonyl)-1H-pyrazol-1-yl)methyl)benzoate (150 mg, 0.17 mmol) in MeOH (4 mL) was added 40% Pd(OH)₂ (60 mg) and 40% Pd/C (60 mg). The reaction was stirred under a H₂ atmosphere at 50° C. for 6 h. The catalyst was removed by filtration through celite and the filtrate concentrated to afford tert-butyl 2-((4-(8-(((2-(difluoromethoxy)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (140 mg, 98.6%) as a white solid. LCMS m/z=869.3 [M+H]⁺; ¹H NMR (DMSO-d₆, 400 MHz) δ 8.24 (d, J=10.2 Hz, 1H), 7.85 (d, J=9.4 Hz, 2H), 7.56-7.14 (m, 6H), 6.89-6.78 (m, 1H), 5.69 (s, 2H), 4.51 (d, J=6.4 Hz, 2H), 3.95 (s, 1H), 3.88-3.47 (m, 7H), 2.98 (s, 1H), 2.04-2.00 (m, 1H), 1.95 (s, 2H), 1.81-1.68 (m, 2H), 1.64-1.59 (m, 2H), 1.54 (d, J=4.2 Hz, 9H), 1.23 (s, 4H), 1.13 (s, 2H)

Step 4: 2-((4-(8-(((2-(difluoromethoxy)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid To a solution of tert-butyl 2-((4-(8-(((2-(difluoromethoxy)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (160 mg, 0.18 mmol) in DCM (3 mL) was added TFA (1.5 mL). The reaction mixture was stirred at room temperature for 2 h. The solvent was removed under vacuum to afford crude. The residue obtained was purified by prep-HPLC to afford 2-((4-(8-(((2-(difluoromethoxy)-3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid (57 mg, 38%) as a white solid. LCMS m/z=813.4 [M+H]⁺; ¹H NMR (DMSO-d₆, 400 MHz) δ 8.29 (d, J=7.6 Hz, 1H), 7.93 (d, J=7.8 Hz, 1H), 7.85 (d, J=13.6 Hz, 1H), 7.51-7.39 (m, 2H), 7.33-7.24 (m, 3H), 6.76 (d, J=7.8 Hz, 1H), 5.75 (d, J=4.0 Hz, 2H), 4.51 (d, J=6.6 Hz, 2H), 4.21-4.12 (m, 1H), 3.99-3.82 (m, 3H), 3.75-3.54 (m, 6H), 3.02-2.87 (m, 1H), 2.68-2.54 (m, 2H), 1.97 (d, J=14.0 Hz, 2H), 1.82-1.66 (m, 2H), 1.65-1.55 (m, 3H), 1.41-1.08 (m, 5H)

TABLE 13
The compounds listed in Table 13 were synthesized from tert-butyl 2-((4-(8-
(hydroxymethyl)-2-(1-(trifluoromethyl)cyclopropane-1-carbonyl)-2,6-diazaspiro[3.4]octane-6-
carbonyl)-1H-pyrazol-1-yl)methyl)benzoate according to the procedures outlined for I′-74 using
the appropriate commercially available reagents and/or intermediates described elsewhere.

| Cmpd. | | | |
|---|---|---|---|
| No | Compound | ¹HNMR | LCMS |
| I-114 | 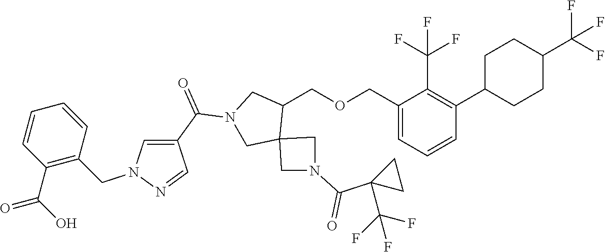 [SMILES: O=C(O)c1ccccc1Cn1cc(C(=O)N2CC(COCc3cccc(C4CCC(C(F)(F)F)CC4)c3C(F)(F)F)C3(C2)CN(C(=O)C2(C(F)(F)F)CC2)C3)cn1] | ¹H NMR (400 MHz, DMSO-d6) δ 8.33 (s, 1H), 7.80 (d, J = 16.2 Hz, 2H), 7.55-7.50 (m, 1H), 7.47- 7.38 (m, 2H), 7.26 (m, 2H), 6.74 (s, 1H), 5.76 (s, 2H), 4.64 (s, 2H), 4.26- 4.08 (m, 2H), 3.95 (s, 2H), 3.86-3.78 (m, 1H), 3.74-3.62 (m, 3H), 3.58- 3.50 (m, 3H), 2.98- 2.87 (m, 1H), 2.01 (t, 3H), 1.80-1.58 (m, 6H), 1.23 (s, 2H), 1.10 (s, 2H). | m/z = 815.4 [M + H]⁺ |

### 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid I′-68

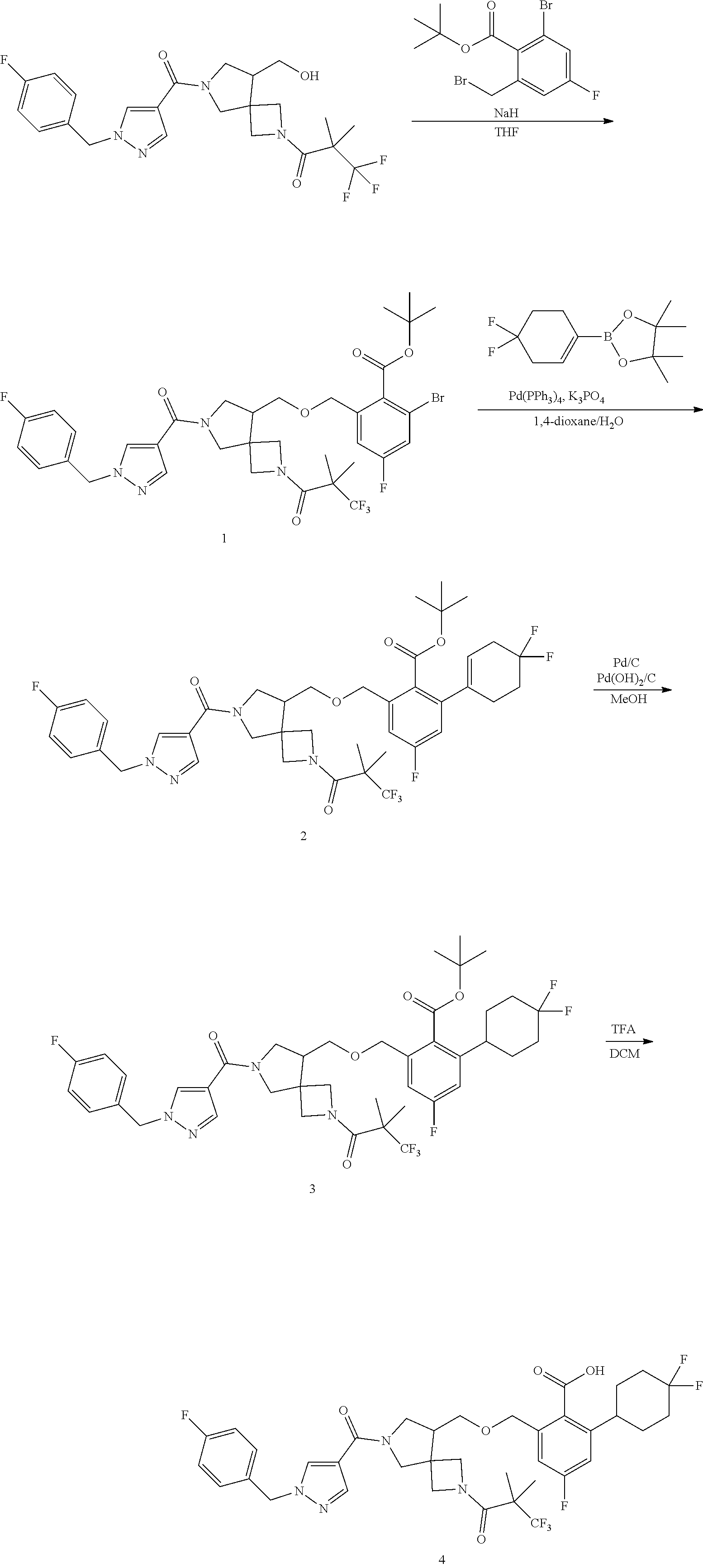

Step 1: tert-butyl 2-bromo-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of 3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one prepared as shown during the synthesis of I′-66 (150 mg, 0.31 mmol) and tert-butyl 2-bromo-4-fluoro-6-methylbenzoate (136 mg, 0.37 mmol) in THF (3 mL) at 0° C. was added 60% NaH (48 mg, 1.24 mmol). The reaction was stirred 20 h at room temperature. The reaction mixture was diluted with water (30 mL) and extracted with EtOAc (20 mL×3). The combined organic layers were washed with brine, dried over Na₂SO₄, filtered and concentrated. The mixture was purified by prep-TLC (eluent: DCM:MeOH=15:1) to afford tert-butyl 2-bromo-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (176 mg, 73%) as a white oil. LCMS m/z=769.2 [M+H]⁺; ¹HNMR (400 MHz, CD3OD) δ 8.21 (d, J=6.1 Hz, 1H), 7.91 (s, 1H), 7.43-7.29 (m, 3H), 7.25-7.16 (m, 1H), 7.13-7.03 (m, 2H), 5.36 (s, 2H), 4.61-3.48 (m, 12H), 2.71-2.58 (m, 1H), 1.58 (d, J=12.4 Hz, 9H), 1.42-1.35 (m, 6H).

Step 2: tert-butyl 4′,4′,5-trifluoro-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-2′,3′,4′,5′-tetrahydro-[1,1′-biphenyl]-2-carboxylate To a solution of tert-butyl 2-bromo-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (170 mg, 0.22 mmol) in 1,4-dioxane (2 mL) and H₂O (0.5 mL) was added 2-(4,4-difluorocyclohex-1-en-1-yl)-4,4,5,5-tetramethyl-1,3,2-dioxaborolane (63 mg, 0.26 mmol), K₃PO₄ (93 mg, 0.44 mmol) and Pd(PPh₃)₄ (23 mg, 0.02 mmol). The reaction was stirred at 100° C. under N₂ atmosphere for 3 h. The mixture was diluted with water (20 mL) and extracted with EtOAc (20 mL×3). The combined organic layers were washed with brine, dried over Na₂SO₄, filtered and concentrated. The residue was purified by prep-TLC (eluent: DCM:MeOH=15:1) to afford tert-butyl 4′,4′,5-trifluoro-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-2′,3′,4′,5′-tetrahydro-[1,1′-biphenyl]-2-carboxylate (169 mg, 95%) as a yellow solid. LCMS m/z=807.2 [M+H]⁺; ¹HNMR (400 MHz, CD3OD) δ 8.21 (s, 1H), 7.91 (s, 1H), 7.68-7.53 (m, 4H), 7.37-7.27 (m, 2H), 5.49-5.46 (m, 1H), 5.36 (s, 2H), 4.64-3.59 (m, 12H), 2.70-2.54 (m, 5H), 2.22-2.07 (m, 2H), 1.51 (d, J=9.8 Hz, 9H), 1.42-1.34 (m, 6H).

Step 3: tert-butyl 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of tert-butyl 4′,4′,5-trifluoro-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-2′,3′,4′,5′-tetrahydro-[1,1′-biphenyl]-2-carboxylate (80 mg, 0.1 mmol) in MeOH (5 mL) was added Pd/C (32 mg) and Pd(OH)₂/C (32 mg). The reaction mixture was stirred under a H₂ atomosphere for 35 h. The catalyst was removed by filtration through celite and the filtrate was concentrated to afford tert-butyl 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (65 mg, 81%) as a black solid. LCMS m/z=809.5 [M+H]⁺; ¹HNMR (400 MHz, CD3OD) δ 8.20 (s, 1H), 7.95-7.88 (m, 1H), 7.37-7.29 (m, 2H), 7.13-6.99 (m, 4H), 5.36 (s, 2H), 4.60-3.46 (m, 12H), 2.87-2.58 (m, 2H), 2.25-2.10 (m, 2H), 1.99-1.72 (m, 6H), 1.59 (d, J=11.1 Hz, 9H), 1.40-1.33 (m, 6H).

Step 4: 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid To a solution of tert-butyl 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (65 mg, 0.08 mmol) in DCM (4 mL) was added TFA (1 mL). The reaction mixture was stirred at room temperature for 4 h. The solvent was removed and the residue was purified by prep-TLC to afford 2-(4,4-difluorocyclohexyl)-4-fluoro-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (50 mg, 83%) as a white solid. LCMS m/z=753.4 [M+H]⁺; ¹HNMR (400 MHz, CD3OD) δ 8.24 (d, J=22.5 Hz, 1H), 7.94-7.88 (m, 1H), 7.36-7.28 (m, 2H), 7.11-6.95 (m, 4H), 5.36 (s, 2H), 4.60 (s, 2H), 4.50-3.43 (m, 10H), 2.99-2.86 (m, 1H), 2.73-2.57 (m, 1H), 2.20-2.06 (m, 2H), 2.00-1.70 (m, 6H), 1.42-1.34 (m, 6H).

TABLE 14
The compounds listed in Table 14 were synthesized from 3,3,3-trifluoro-1-
(6-(1-(4-fluorobenzyl)-1H-pyrazole-4-carbonyl)-8-(hydroxymethyl)-2,6-diazaspiro[3.4]octan-2-
yl)-2,2-dimethylpropan-1-one according to the procedures outlined for I′-68 using the appropriate
commercially available reagents and/or intermediates described elsewhere.

| Cmpd. | | | |
|---|---|---|---|
| No. | Compound | ¹HNMR | LCMS |
| I′-76 | 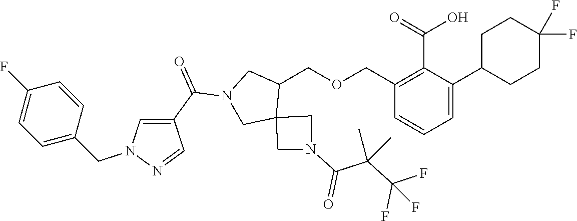 [SMILES: CC(C)(C(=O)N1CC2(CN(C(=O)c3cnn(Cc4ccc(F)cc4)c3)CC2COCc2cccc(C3CCC(F)(F)CC3)c2C(=O)O)C1)C(F)(F)F] | ¹H NMR (400 MHz, Methanol-d₄) δ 8.22- 8.09 (m, 1H), 7.89 (s, 1H), 7.36-7.19 (m, 5H), 7.06 (t, J = 8.4 Hz, 2H), 5.34 (s, 2H), 4.59 (s, 2H), 4.44-3.43 (m, 10H), 2.91-2.76 (m, 1H), 2.72- 2.54 (m, 1H), 2.19- 2.05 (m, 2H), 1.96-1.71 (m, 6H), 1.36 (s, 6H). | m/z = 735.3 [M + H]⁺ |

### (8-(((3-(4,4-difluorocyclohexyl)-2-(2H-tetrazol-5-yl)benzyl)oxy)methyl)-2-((S)-2,2-dimethylcyclopropane-1-carbonyl)-2,6-diazaspiro[3.4]octan-6-yl)(1-(4-fluorobenzyl)-1H-pyrazol-4-yl)methanone I′-70

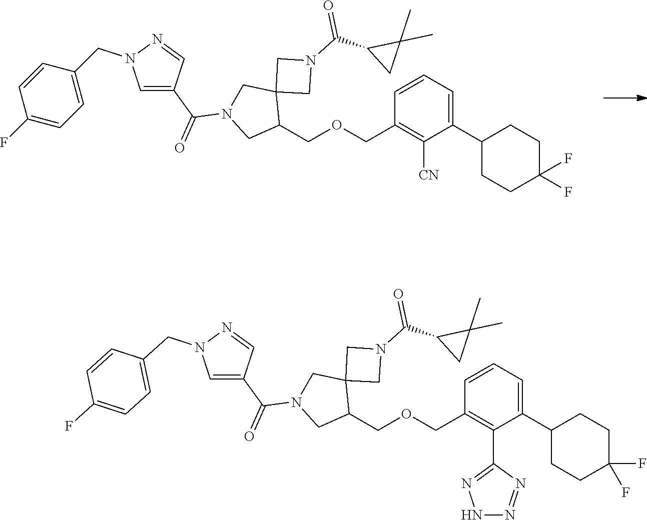 [SMILES: CC1(C)C[C@@H]1C(=O)N1CC2(CN(C(=O)c3cnn(Cc4ccc(F)cc4)c3)CC2COCc2cccc(C3CCC(F)(F)CC3)c2C#N)C1 ; CC1(C)C[C@@H]1C(=O)N1CC2(CN(C(=O)c3cnn(Cc4ccc(F)cc4)c3)CC2COCc2cccc(C3CCC(F)(F)CC3)c2-c2nn[nH]n2)C1]

To a solution of 2-(4,4-difluorocyclohexyl)-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile (55 mg, 0.08 mmol) in toluene (2 mL) was added azidotrimethylsilane (28 mg, 0.25 mmol) and dibutylstannanone (8 mg, 0.03 mmol). The reaction mixture was stirred at 150° C. in a sealed tube for 6 h. The mixture was diluted with water (20 mL) and extracted with EtOAc (20 mL×3). The combined organic layers were washed with brine, dried over Na₂SO₄, filtered and concentrated. The residue obtained was purified by prep-HPLC to afford (8-(((3-(4,4-difluorocyclohexyl)-2-(2H-tetrazol-5-yl)benzyl)oxy)methyl)-2-((S)-2,2-dimethylcyclopropane-1-carbonyl)-2,6-diazaspiro[3.4]octan-6-yl)(1-(4-fluorobenzyl)-1H-pyrazol-4-yl)methanone (24 mg, 28%) as a yellow solid. LCMS m/z=717.4 [M+H]⁺; ¹H NMR (400 MHz, DMSO-d₆) δ 8.34 (s, 1H), 7.82 (s, 1H), 7.54-7.30 (m, 5H), 7.22-7.13 (m, 2H), 5.35 (d, J=7.8 Hz, 2H), 4.22-3.36 (m, 11H), 2.24-1.84 (m, 4H), 1.79-1.59 (m, 5H), 1.39-1.28 (m, 1H), 1.13-0.96 (m, 6H), 0.89-0.81 (m, 1H), 0.70-0.63 (m, 1H).

TABLE 15
The compounds listed in Table 15 were synthesized from ((S)-2-((S)-2,2-
dimethylcyclopropane-1-carbonyl)-8-(hydroxymethyl)-2,6-diazaspiro[3.4]octan-6-yl)(1-(4-
fluorobenzyl)-1H-pyrazol-4-yl)methanone according to the procedures outlined for I′-70 using the
appropriate commercially available reagents and/or intermediates described elsewhere.

| Cmpd. | | | |
|---|---|---|---|
| No. | Compound | ¹HNMR | LCMS |
| I′-87 | 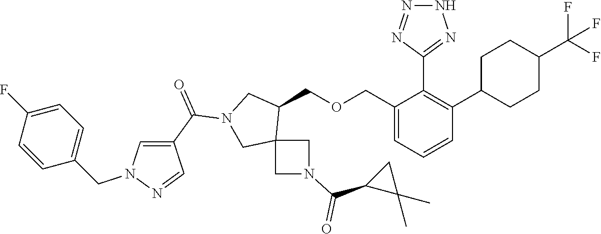 [SMILES: CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2-c2nn[nH]n2)C1] | 1H NMR (400 MHz, Methanol-d4) δ 8.37- 8.15 (m, 1H), 7.95-7.85 (m, 1H), 7.57-7.25 (m, 5H), 7.12-7.01 (m, 2H), 5.46-5.31 (m, 2H), 4.32- 3.36 (m, 12H), 2.52- 2.26 (m, 2H), 2.18-2.07 (m, 1H), 2.07-1.94 (m, 2H), 1.79-1.31 (m, 7H), 1.19-0.98 (m, 7H), 0.80- 0.66 (m, 1H). | m/z = 748.3 [M + H]⁺ |

### 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoic acid I-88

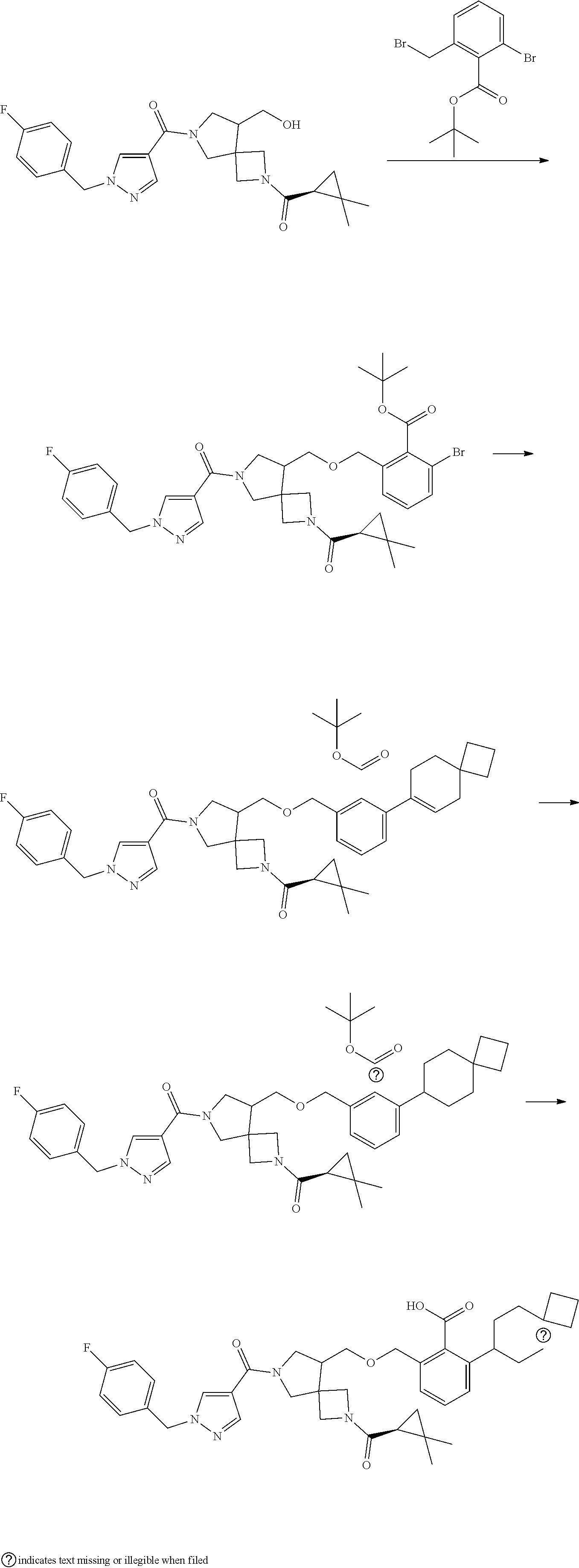

Step 1: tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate To a solution of (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (240 mg, 0.55 mmol) and tert-butyl 2-bromo-6-(bromomethyl)benzoate (230 mg, 0.66 mmol) in THF at 0° C. was added sodium hydride (240 mg, 6 mmol). Then the mixture was warmed to room temperature and stirred at RT for 3 h. The mixture was diluted with water (30 mL) and extracted with EtOAc (70 mL×2). The combined organic layers were dried over Na₂SO₄, filtered and concentrated. The mixture was purified by prep-TLC (eluent: DCM:MeOH=20:1) to afford tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (340 mg, 87% yield) as a white solid. LCMS m/z=709.0 [M+H]⁺; ¹H NMR (400 MHz, Chloroform-d) δ 7.97-7.77 (m, 2H), 7.56-7.47 (m, 1H), 7.34-7.27 (m, 2H), 7.25-7.18 (m, 2H), 7.08-7.00 (m, 2H), 5.29 (s, 2H), 4.64-4.46 (m, 2H), 4.15-4.06 (m, 1H), 3.97-3.89 (m, 2H), 3.87-3.74 (m, 3H), 3.65-3.53 (m, 2H), 3.52-3.44 (m, 2H), 2.61-2.49 (m, 1H), 1.59 (s, 9H), 1.22-1.18 (m, 1H), 1.16-1.12 (m, 6H), 1.03-0.92 (m, 1H), 0.78-0.70 (m, 1H).

Step 2: tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]non-6-en-7-yl)benzoate To a solution of tert-butyl 2-bromo-6-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (340 mg, 0.48 mmol) in 1,4-dioxane (4 mL) and H₂O (1 mL) was added 4,4,5,5-tetramethyl-2-(spiro[3.5]non-6-en-7-yl)-1,3,2-dioxaborolane (142 mg, 0.58 mmol), K₂CO₃ (199 mg, 1.44 mmol) and Pd(PPh₃)₄ (55 mg, 0.048 mmol). The reaction was stirred at 100° C. under N₂ atmosphere for 2 h. The mixture was diluted with water (20 mL) and extracted with EtOAc (40 mL×2). The combined organic layers were dried over Na₂SO₄, filtered and concentrated. The residue was purified by RP-column to afford tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]non-6-en-7-yl)benzoate (85 mg, 24% yield) as a yellow solid. LCMS m/z=751.4 [M+H]⁺; ¹H NMR (400 MHz, Chloroform-d) δ 7.93-7.75 (m, 2H), 7.25-7.17 (m, 4H), 7.14-6.98 (m, 3H), 5.52 (s, 1H), 5.27 (s, 2H), 4.64-4.48 (m, 2H), 4.19-4.07 (m, 2H), 3.97-3.73 (m, 5H), 3.66-3.43 (m, 3H), 2.60-2.46 (m, 1H), 2.35-2.28 (m, 2H), 2.19-2.14 (m, 2H), 1.92-1.86 (m, 2H), 1.85-1.77 (m, 4H), 1.75-1.70 (m, 2H), 1.51 (s, 9H), 1.16-1.09 (m, 7H), 0.91-0.85 (m, 111), 0.77-0.69 (m, 1H).

Step 3: tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoate To a solution of tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]non-6-en-7-yl)benzoate (85 mg, 1.13 mmol) in MeOH (2 mL) was added 10% Pd/C (34 mg). The reaction was stirred at room temperature under H₂ atmosphere for 2 days. The mixture was filtered through celite and concentrated to afford the crude tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoate (65 mg crude, 76% yield) as a colorless oil, which was used in the next step directly. LCMS m/z=753.4 [M+H]⁺.

Step 4: 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoic acid I-88 To a solution of tert-butyl 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoate (65 mg, 0.086 mmol) in DCM (2 mL) was added TFA (1 mL). The reaction mixture was stirred at room temperature for 2 h. The solvent was removed under vacuum and purified by prep-TLC (eluent: DCM:MeOH=20:1) to afford 2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(spiro[3.5]nonan-7-yl)benzoic acid (I-88, 36 mg, 60% yield) as a white solid. LCMS m/z=697.4 [M+H]⁺; ¹H NMR (400 MHz, DMSO-d₆) δ 8.43-8.30 (m, 1H), 7.86-7.78 (m, 1H), 7.38-7.30 (m, 2H), 7.25-7.11 (m, 5H), 5.34 (s, 2H), 4.47 (s, 2H), 4.30-3.49 (m, 12H), 1.87-1.75 (m, 6H), 1.72-1.55 (m, 4H), 1.48-1.23 (m, 5H), 1.12-0.97 (m, 6H), 0.89-0.82 (m, 1H), 0.70-0.57 (m, 1H).

### 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoic acid I-119

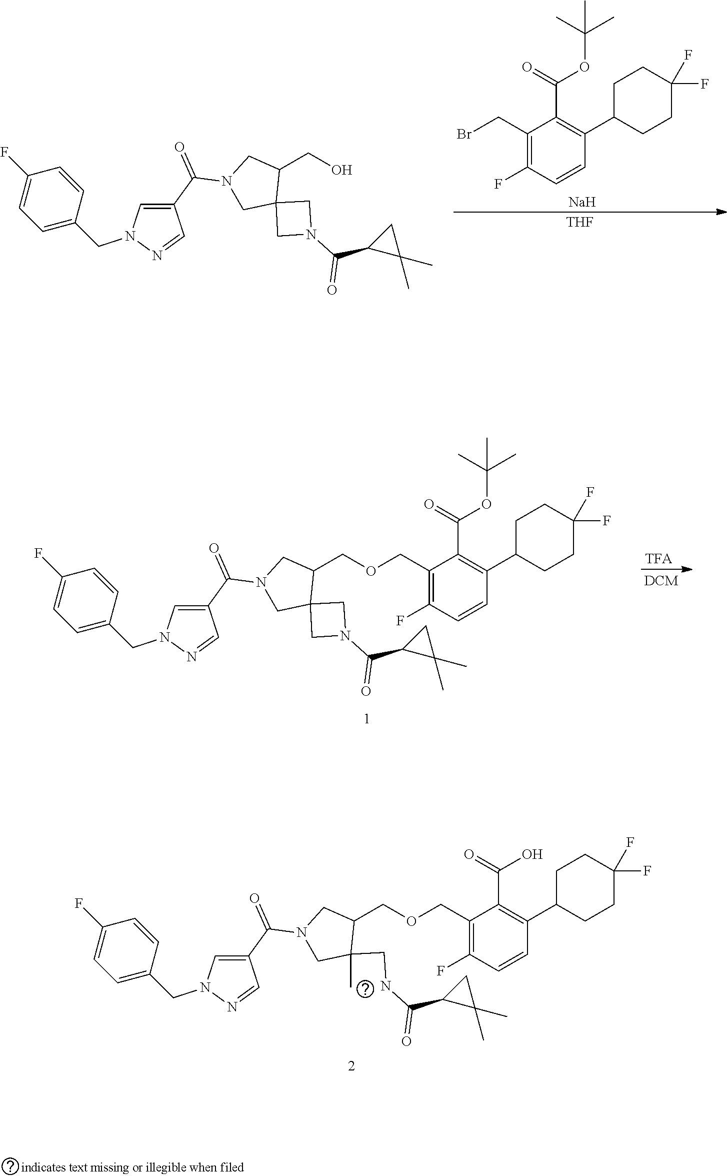

Step 1: tert-butyl 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoate To a solution of tert-butyl 2-(bromomethyl)-6-(4,4-difluorocyclohexyl)-3-fluorobenzoate (85 mg, 0.19 mmol), (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (94 mg, 0.23 mmol) in THF (1 mL) was added and NaH (15 mg, 0.58 mmol). The mixture was stirred at room temperature for 22 h. The mixture was diluted with water (10 mL) and extracted with EtOAc (10 mL×2). The combined organic layers were washed with brine, dried over Na₂SO₄, filtered and concentrated. The residue was purified by prep-TLC (eluent: DCM:MeOH=20:1, v/v) to afford tert-butyl 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoate (60 mg, 34%) as a colorless oil. LCMS m/z=767.4 [M+H]⁺; ¹H NMR (400 MHz, Methanol-d₄) δ 8.20 (d, J=10.2 Hz, 1H), 7.89 (d, J=10.8 Hz, 1H), 7.39-7.26 (m, 3H), 7.09 (d, J=8.6 Hz, 3H), 5.36 (d, J=4.6 Hz, 2H), 4.61 (d, J=9.2 Hz, 2H), 4.52-3.62 (m, 9H), 3.55-3.43 (m, 1H), 2.72 (s, 1H), 2.56 (s, 1H), 2.15 (s, 2H), 1.84 (d, J=32.2 Hz, 4H), 1.66-1.50 (m, 9H), 1.20-1.06 (m, 9H), 1.03 (d, J=4.6 Hz, 1H), 0.76 (s, 1H).

Step 2: 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoic acid I-119 To a solution of tert-butyl 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoate (60 mg, 0.78 mmol) in DCM (2 mL) was added TFA (1 mL). The reaction mixture was stirred at room temperature for 1 h. The solvent was removed under vacuum. The residue was purified by prep-HPLC to afford 6-(4,4-difluorocyclohexyl)-2-(((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-3-fluorobenzoic acid (18 mg, 32%) as a white solid. LCMS m/z=711.4 [M+H]⁺; ¹H NMR (400 MHz, Methanol-d₄) δ 8.30-8.13 (m, 1H), 7.90 (d, J=14.0 Hz, 1H), 7.42-7.27 (m, 3H), 7.23-6.98 (m, 3H), 5.35 (d, J=3.4 Hz, 2H), 4.66 (dd, J=11.8, 7.6 Hz, 2H), 4.39 (s, 1H), 4.28-3.44 (m, 10H), 2.81 (s, 1H), 2.74-2.53 (m, 1H), 2.11 (s, 2H), 1.99-1.69 (m, 6H), 1.38 (dd, J=13.8, 7.0 Hz, 2H), 1.20-1.09 (m, 4H), 1.09-0.99 (m, 3H), 0.75 (dd, J=12.6, 7.2 Hz, 1H).

### (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoic acid I′-66

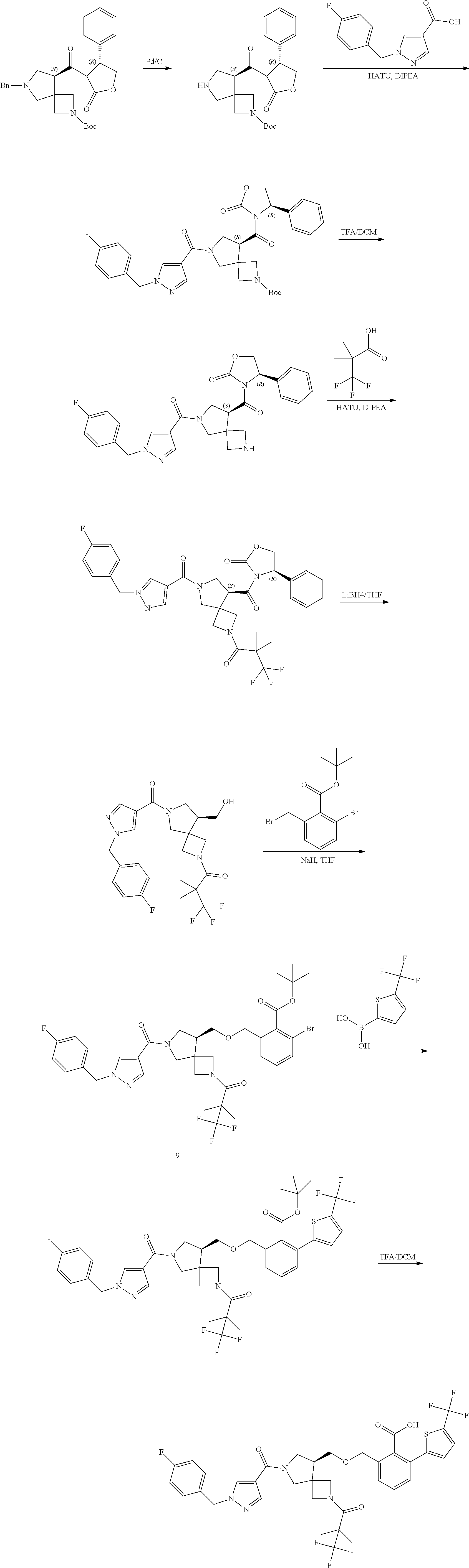

Step 1: tert-butyl (S)-8-((R)-2-oxo-4-phenyloxazolidine-3-carbonyl)-2,6-diazaspiro[3.4]octane-2-carboxylate To a solution of tert-butyl (S)-6-benzyl-8-((R)-2-oxo-4-phenyloxazolidine-3-carbonyl)-2,6-diazaspiro[3.4]octane-2-carboxylate (1 g, 2 mmol) in EtOAc (8 mL) was added Pd/C (400 mg). The reaction mixture was stirred at 50° C. for 36 h with H₂ balloon. The mixture was filtrated with MeOH and concentrated under vacuum to afford tert-butyl (S)-8-((R)-2-oxo-4-phenyloxazolidine-3-carbonyl)-2,6-diazaspiro[3.4]octane-2-carboxylate (0.7 g, 88%) as a yellow oil. LCMS m/z=402.2 [M+H]⁺.

Step 2: tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate To a solution of 1-(4-fluorobenzyl)-1H-pyrazole-4-carboxylic acid (1.9 g, 8.7 mmol) and HATU (3.3 g, 8.7 mmol) in DCM (50 mL) was added DIPEA (3.4 g, 26.1 mmol). The mixture was stirred at room temperature for 30 min, tert-butyl (S)-8-((R)-2-oxo-4-phenyloxazolidine-3-carbonyl)-2,6-diazaspiro[3.4]octane-2-carboxylate (3.5 g, 8.7 mmol) was added and stirred at room temperature for another 2 h. The mixture was diluted with water (80 mL), extracted with DCM (100 mL×2) and the combined organic layers washed with brine, dried over Na₂SO₄ and filtered. The solvent was removed and the residue purified by column chromatography on silica gel (eluent: DCM:MeOH=50:1) to afford tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate (2.3 g, 43%) as a yellow oil. LCMS m/z=604.3 [M+H]⁺; ¹H NMR (400 MHz, DMSO-d₆) δ 8.35-8.27 (m, 1H), 7.81-7.74 (m, 1H), 7.39-7.05 (m, 9H), 5.47-5.27 (m, 3H), 4.80-4.70 (m, 1H), 4.34-3.57 (m, 10H), 1.38 (s, 9H).

Step 3: (R)-3-((S)-6-(1-(4-fluorobenzyl)-1H-pyrazole-4-carbonyl)-2,6-diazaspiro[3.4]octane-8-carbonyl)-4-phenyloxazolidin-2-one To a solution of tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate (2.3 g, 3.8 mmol) in DCM (4 mL) was added TFA (2 ml). The reaction mixture was stirred at room temperature for 2 h. The mixture was concentrated in vacuo to afford (R)-3-((S)-6-(1-(4-fluorobenzyl)-1H-pyrazole-4-carbonyl)-2,6-diazaspiro[3.4]octane-8-carbonyl)-4-phenyloxazolidin-2-one (1.9 g, 100%) as a yellow oil. LCMS m/z=504.2 [M+H]⁺.

Step 4: (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one To a solution of 3,3,3-trifluoro-2,2-dimethylpropanoic acid (588 mg, 3.8 mmol) and HATU (1.4 g, 3.8 mmol) in DCM (20 mL) was added DTPEA (1.5 g, 15.2 mmol). The mixture was stirred at room temperature for 30 min. (R)-3-((S)-6-(1-(4-fluorobenzyl)-1H-pyrazole-4-carbonyl)-2,6-diazaspiro[3.4]octane-8-carbonyl)-4-phenyloxazolidin-2-one (1.9 g, 3.8 mmol) was added and stirred at room temperature for another 2 h. The mixture was diluted with water (80 mL), extracted with DCM (100 mL×2) and the combined organic layers washed with brine, dried over Na₂SO₄ and filtered. The solvent was removed and the residue purified by column chromatography on silica gel (eluent: DCM:MeOH=40:1) to afford (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one (1.2 g, 50%) as white solid. LCMS m/z=642.2 [M+H]⁺; ¹H NMR (400 MHz, Chloroform-d) δ 7.79-7.65 (m, 2H), 7.39-7.28 (m, 2H), 7.25-7.09 (m, 5H), 7.05 (t, J=8.4 Hz, 2H), 5.43-5.37 (m, 1H), 5.29-5.22 (m, 2H), 4.79-4.72 (m, 1H), 4.50-4.25 (m, 4H), 4.11-3.82 (m, 6H), 1.39 (s, 6H).

Step 5: (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one To a solution of (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one (500 mg, 0.78 mmol) in THF (10 mL) at −78° C. was added Lithium borohydride (2 M/L in THF, 273 ul, 0.55 mmol). The reaction mixture was stirred at 0° C. for 4 h then diluted with water (10 mL), extracted with EtOAc (50 mL), the combined organic layers were washed with brine, dried over Na₂SO₄, filtered and concentrated in vacuo. The residue was purified by prep-TLC (eluent: DCM:MeOH=20:1) to afford (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (140 mg, 37%) as a white solid. LCMS m/z=483.2 [M+H]⁺; ¹H NMR (400 MHz, DMSO-d₆) δ 8.35-8.31 (m, 1H), 7.81 (d, J=10.4 Hz, 1H), 7.35-7.30 (m, 2H), 7.18 (t, J=8.8 Hz, 2H), 5.34 (s, 2H), 4.81-4.76 (m, 1H), 4.40-3.44 (m, 10H), 2.03-1.96 (m, 1H), 1.36-1.32 (m, 6H).

Step 6: tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (100 mg, 0.21 mmol) and tert-butyl 2-bromo-6-(bromomethyl)benzoate (74 mg, 0.21 mmol) in THF at 0° C. was added sodium hydride (42 mg, 1.1 mmol). Then the mixture was warmed to room temperature and stirred at RT for 3 h. The mixture was diluted with water (30 mL) and extracted with EtOAc (70 mL×2). The combined organic layers were dried over Na₂SO₄, filtered and concentrated. The mixture was purified by prep-TLC (eluent: DCM:MeOH=30:1) to afford tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (140 mg, 70% yield) as a white solid. LCMS m/z=751.2 [M+H]⁺; ¹H NMR (400 MHz, Chloroform-d) δ 8.01-7.73 (m, 2H), 7.56-7.45 (m, 1H), 7.25-7.14 (m, 4H), 7.08-6.97 (m, 2H), 5.27 (s, 2H), 4.67-3.68 (m, 9H), 3.63-3.39 (m, 3H), 2.58-2.46 (m, 1H), 1.58 (s, 9H), 1.37 (s, 6H).

Step 7: tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoate To a solution of tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (120 mg, 0.16 mmol) in 1,4-dioxane (6 mL) and H₂O (2 mL) was added (5-(trifluoromethyl)thiophen-2-yl)boronic acid (31 mg, 0.16 mmol), K₂CO₃ (18 mg, 0.32 mmol) and Pd(PPh₃)₄ (44 mg, 0.016 mmol). The reaction was stirred at 100° C. under N2 atmosphere for 6 h. The mixture was diluted with water (20 mL) and extracted with EtOAc (40 mL×2). The combined organic layers were dried over Na₂SO₄, filtered and concentrated. The residue was purified by prep-TLC (eluent: DCM:MeOH=20:1) to afford tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoate (90 mg, 69% yield) as a yellow solid. LCMS m/z=767.2 [M+H−56]⁺.

Step 8: (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoic acid I′-66 To a solution of tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoate (80 mg, 0.08 mmol) in DCM (2 mL) was added TFA (1 ml). The reaction mixture was stirred at room temperature for 2 h. The mixture was purified by prep-HPLC to afford (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-2-yl)benzoic acid (30 mg, 42%) as a white solid. LCMS m/z=767.2 [M+H]⁺; ¹H NMR (400 MHz, DMSO-d₆) δ 8.33 (s, 1H), 7.81 (d, J=13.8 Hz, 1H), 7.72 (s, 1H), 7.48 (d, J=5.8 Hz, 3H), 7.31 (s, 2H), 7.26-7.11 (m, 3H), 5.33 (d, J=14.0 Hz, 2H), 4.58 (s, 2H), 4.42-3.37 (m, 10H), 2.67 (s, 1H), 1.32 (s, 6H).

TABLE 16
The compounds listed in Table 16 were synthesized from tert-butyl (S)-2-
bromo-6-(((6-(1-(4-fluorobenzyl)-1H-pyrazole-4-carbonyl)-2-(3,3,3-trifluoro-2,2-
dimethylpropanoyl)-2,6-diazaspiro[3.4]octan-8-yl)methoxy)methyl)benzoate according to the
procedures outlined for I′-66 using the appropriate commercially available reagents and/or
intermediates described elsewhere.

| Cmpd. | | | |
|---|---|---|---|
| No. | Compound | ¹HNMR | LCMS |
| I′-37 | 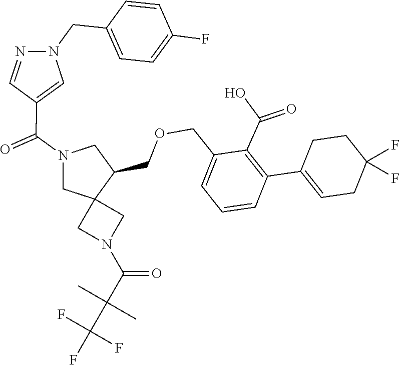 [SMILES: CC(C)(C(=O)N1CC2(CN(C(=O)c3cnn(Cc4ccc(F)cc4)c3)C[C@H]2COCc2cccc(C3=CCC(F)(F)CC3)c2C(=O)O)C1)C(F)(F)F] | ¹H NMR (400 MHz, DMSO-d₆) δ 8.55-8.50 (m, 1H), 7.81 (d, J = 21.7 Hz, 1H), 7.36 (d, J = 21.2 Hz, 2H), 7.15 (dt, J = 19.0, 9.5 Hz, 3H), 6.91 (t, J = 11.2 Hz, 2H), 5.50 (s, 1H), 5.35 (s, 2H), 4.67 4.39 (m, 3H), 4.37 − 3.89 (m, 3H), 3.67 (t, J = 59.0 Hz, 6H), 2.58 (s, 5H), 2.00 (s, 2H), 1.33 (d, J = 6.4 Hz, 6H). | m/z = 733.2 [M + H]⁺ |
| I′-28 | 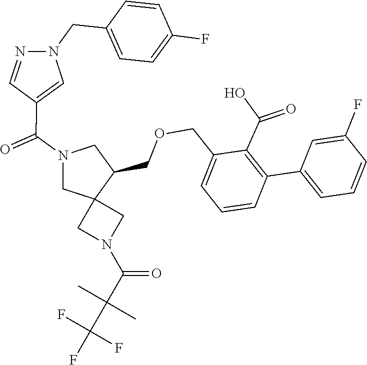 [SMILES: CC(C)(C(=O)N1CC2(CN(C(=O)c3cnn(Cc4ccc(F)cc4)c3)C[C@H]2COCc2cccc(-c3cccc(F)c3)c2C(=O)O)C1)C(F)(F)F] | ¹H NMR (400 MHz, DMSO-d₆) δ 8.39 (d, J = 31.8 Hz, 1H), 7.81 (d, J = 17.9 Hz, 1H), 7.46 − 7.22 (m, 8H), 7.21 − 7.10 (m, 3H), 5.32 (d, J = 21.8 Hz, 2H), 4.57 (s, 2H), 4.40 − 3.38 (m, 10H), 2.69 − 2.53 (m, 1H), 1.32 (d, J = 6.0 Hz, 6H). | m/z = 711.4 [M + H]⁺ |
| I′-12 | 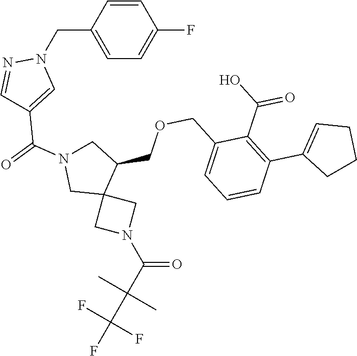 [SMILES: CC(C)(C(=O)N1CC2(CN(C(=O)c3cnn(Cc4ccc(F)cc4)c3)C[C@H]2COCc2cccc(C3=CCCC3)c2C(=O)O)C1)C(F)(F)F] | ¹H NMR (400 MHz, DMSO-d₆) δ 8.37 (d, J = 23.8 Hz, 1H), 7.81 (d, J = 13.2 Hz, 1H), 7.36 − 7.31 (m, 2H), 7.28 − 7.12 (m, 5H), 5.92 (s, 1H), 5.34 (s, 2H), 4.61 − 3.39 (m, 13H), 2.65 − 2.59 (m, 2H), 2.45 − 2.37 (m, 2H), 1.89 (q, J = 7.6 Hz, 2H), 1.31 (d, J = 6.0 Hz, 6H). | m/z = 683.4 [M + H]⁺ |
| I′-88 | 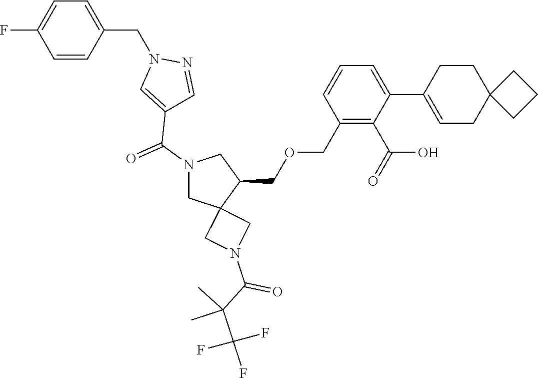 [SMILES: CC(C)(C(=O)N1CC2(CN(C(=O)c3cnn(Cc4ccc(F)cc4)c3)C[C@H]2COCc2cccc(C3=CCC4(CCC4)CC3)c2C(=O)O)C1)C(F)(F)F] | ¹H NMR (400 MHz, DMSO-d₆) δ 12.88 (s, 1H), 8.34 (s, 1H), 7.81 (d, J = 11.8 Hz, 1H), 7.36 − 7.08 (m, 7H), 5.47 (s, 1H), 5.34 (d, J = 4.8 Hz, 2H), 4.60 − 3.36 (m, 13H), 2.30 − 2.24 (m, 2H), 2.17 − 2.10 (m, 2H), 1.88 − 1.62 (m, 8H), 1.31 (s, 6H). | m/z = 737.4 [M + H]⁺ |
| I′-89 | 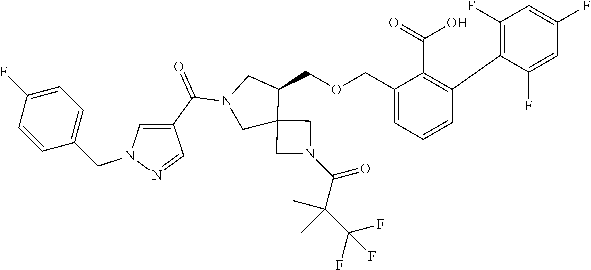 [SMILES: CC(C)(C(=O)N1CC2(CN(C(=O)c3cnn(Cc4ccc(F)cc4)c3)C[C@H]2COCc2cccc(-c3c(F)cc(F)cc3F)c2C(=O)O)C1)C(F)(F)F] | ¹HNMR (400 MHz, Methanol-d4) δ 8.27 (d, J = 33.4 Hz, 1H), 7.93 (s, 1H), 7.57 − 7.42 (m, 2H), 7.39 − 7.24 (m, 3H), 7.14 − 7.02 (m, 2H), 6.96 − 6.83 (m, 2H), 5.42 − 5.28 (m, 2H), 4.83 − 4.70 (m, 2H), 4.61 − 4.23 (m, 2H), 4.14 − 3.49 (m, 8H), 2.74 − 2.61 (m, 1H), 1.43 − 1.35 (m, 6H). | m/z = 747.3 [M + H]⁺ |
| I′-90 | 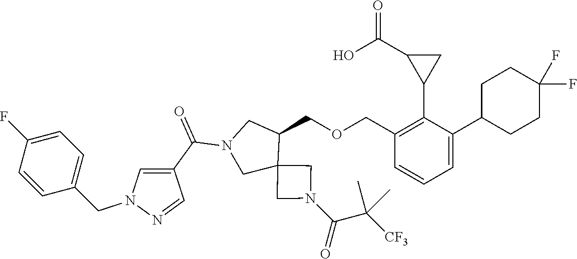 [SMILES: CC(C)(C(=O)N1CC2(CN(C(=O)c3cnn(Cc4ccc(F)cc4)c3)C[C@H]2COCc2cccc(C3CCC(F)(F)CC3)c2C2CC2C(=O)O)C1)C(F)(F)F] | ¹H NMR (400 MHz, CD₃OD) δ 8.19 (s, 1H), 7.89 (s, 1H), 7.38 − 7.27 (m, 2H), 7.22 − 7.01 (m, 5H), 5.35 (s, 2H), 4.76 − 3.45 (m, 13H), 2.75 − 2.56 (m, 1H), 2.43 − 2.29 (m, 1H), 2.21 − 2.09 (m, 2H), 1.97 − 1.59 (m, 8H), 1.38 (s, 6H), 1.27 − 1.15 (m, 1H). | m/z = 775.3 [M + H]⁺ |
| I′-91 | 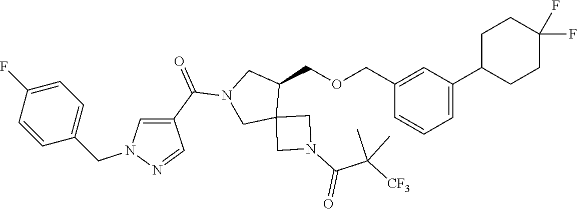 [SMILES: CC(C)(C(=O)N1CC2(CN(C(=O)c3cnn(Cc4ccc(F)cc4)c3)C[C@H]2COCc2cccc(C3CCC(F)(F)CC3)c2)C1)C(F)(F)F] | ¹H NMR (400 MHz, CD₃OD) δ 8.18 (d, J = 7.2 Hz, 1H), 7.89 (d, J = 6.4 Hz, 1H), 7.34 − 7.05 (m, 8H), 5.37 − 5.32 (m, 2H), 4.50 (s, 3H), 4.34 − 4.21 (m, 1H), 4.10 − 3.60 (m, 8H), 2.73 − 2.58 (m, 2H), 2.14 − 2.05 (m, 2H), 1.95 − 1.70 (m, 6H), 1.39 − 1.35 (m, 6H). | m/z 691.3 [M + H]⁺ |
| I′-92 | 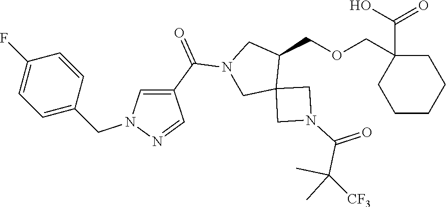 [SMILES: CC(C)(C(=O)N1CC2(CN(C(=O)c3cnn(Cc4ccc(F)cc4)c3)C[C@H]2COCC2(C(=O)O)CCCCC2)C1)C(F)(F)F] | ¹H NMR (400 MHz, Methanol-d₄) δ 8.21 (d, J = 18.9 Hz, 1H), 7.91 (d, J = 16.0 Hz, 1H), 7.32 (d, J = 3.8 Hz, 2H), 7.08 (t, J = 8.6 Hz, 2H), 5.36 (d, J = 3.8 Hz, 2H), 4.78 − 4.18 (m, 4H), 4.13 − 3.66 (m, 4H), 3.50 (d, J = 8.9 Hz, 2H), 2.87 − 2.60 (m, 1H), 2.10 − 1.91 (m, 2H), 1.68 − 1.47 (m, 3H), | m/z = 623.3 [M + H]⁺ |
| | | 1.47 − 1.38 (m, 6H), | |
| | | 1.30 − 1.14 (m, 4H). | |

### 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N—(N,N-dimethylsulfamoyl)benzamide 1′-65

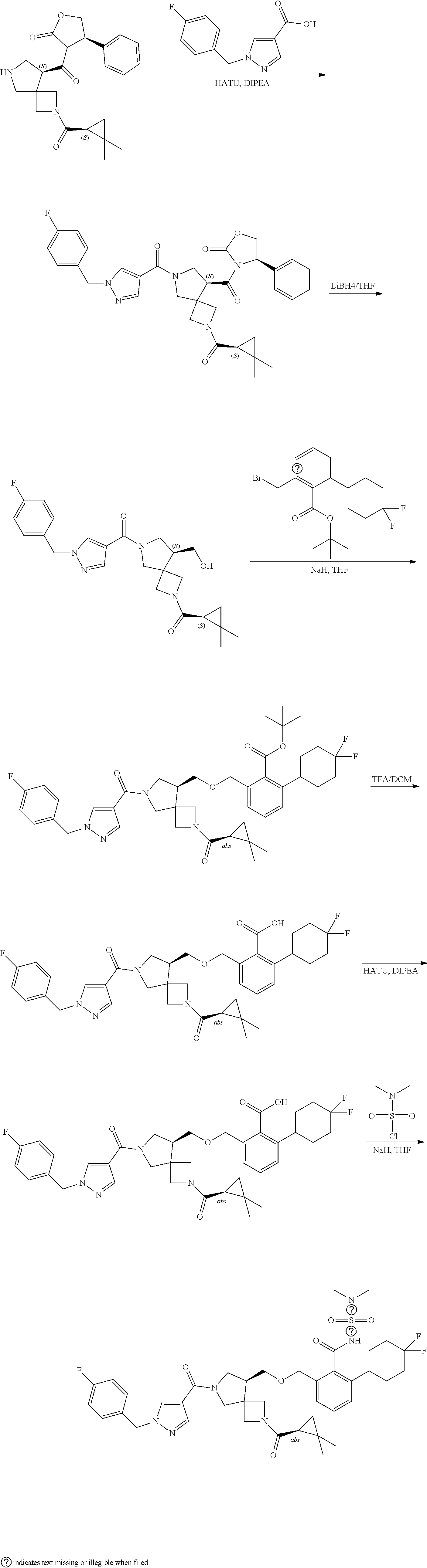

Step 1: (R)-3-((S)-2-((S)-2,2-dimethylcyclopropane-1-carbonyl)-6-(1-(4-fluorobenzyl)-1H-pyrazole-4-carbonyl)-2,6-diazaspiro[3.4]octane-8-carbonyl)-4-phenyloxazolidin-2-one To a solution of 1-(4-fluorobenzyl)-1H-pyrazole-4-carboxylic acid (1.6 g, 7.3 mmol) in DCM (30 mL) was added HATU (2.8 g, 7.3 mmol) and the mixture stirred at room temperature for 30 min. (R)-3-((S)-2-((S)-2,2-dimethylcyclopropane-1-carbonyl)-2,6-diazaspiro[3.4]octane-8-carbonyl)-4-phenyloxazolidin-2-one (2.9 g, 7.3 mmol) and DIPEA (3.77 g, 29.18 mmol) were added and the reaction stirred at room temperature for another 2 h. The mixture was diluted with water (50 mL), extracted with DCM (50 mL×3) and the combined organic layers washed with brine, dried over Na₂SO₄ and filtered. The solvent was removed and the residue purified by column chromatography on silica gel (eluent: DCM:MeOH=80:1) to afford the (R)-3-((S)-2-((S)-2,2-dimethylcyclopropane-1-carbonyl)-6-(1-(4-fluorobenzyl)-1H-pyrazole-4-carbonyl)-2,6-diazaspiro[3.4]octane-8-carbonyl)-4-phenyloxazolidin-2-one (1.2 g, 24%) as a white solid. LCMS m/z=600.3 [M+H]⁺ ; 1H NMR (400 MHz, DMSO-d₆) δ 8.35-8.29 (m, 1H), 7.81-7.75 (m, 1H), 7.38-7.25 (m, 4H), 7.24-7.08 (m, 5H), 5.47-5.42 (m, 1H), 5.39-5.29 (m, 2H), 4.80-4.72 (m, 1H), 4.36-4.04 (m, 5H), 3.90-3.59 (m, 5H), 1.41-1.32 (m, 1H), 1.12-1.03 (m, 6H), 0.89-0.84 (m, 1H), 0.71-0.66 (m, 1H).

Step 2: ((S)-2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone To a solution of (R)-3-((S)-2-((S)-2,2-dimethylcyclopropane-1-carbonyl)-6-(1-(4-fluorobenzyl)-1H-pyrazole-4-carbonyl)-2,6-diazaspiro[3.4]octane-8-carbonyl)-4-phenyloxazolidin-2-one (600 mg, 1 mmol) in dry THF (6 mL) was added LiBH₄ (2 M/L in THF, 0.35 mL, 0.7 mmol) at −78° C. under N₂ atmosphere and the reaction stirred at room temperature overnight. The mixture was diluted with water (50 mL), extracted with EtOAc (20 mL×3) and the combined organic layers washed with brine, dried over Na₂SO₄ and filtered. The solvent was removed and the residue purified by RP-column (42% CAN in water) to afford the ((S)-2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (240 mg, 54.5%) as a white solid. LCMS m/z=441.2 [M+H]⁺; ¹H NMR (400 MHz, DMSO-d₆) δ 8.34 (d, J=6.2 Hz, 1H), 7.82 (d, J=10.0 Hz, 1H), 7.36-7.30 (m, 2H), 7.18 (t, J=8.8 Hz, 2H), 5.34 (s, 2H), 4.80-4.75 (m, 1H), 4.35-4.17 (m, 1H), 4.09-4.01 (m, 1H), 4.00-3.59 (m, 6H), 3.54-3.37 (m, 2H), 2.46-2.23 (m, 1H), 1.40-1.32 (m, 1H), 1.13-1.03 (m, 6H), 0.88-0.83 (m, 1H), 0.69-0.65 (m, 1H).

Step 3: tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate To a solution of ((S)-2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (250 mg, 0.57 mmol) in dry THF (5 mL) were added tert-butyl 2-(bromomethyl)-6-(4,4-difluorocyclohexyl)benzoate (243 mg, 0.62 mmol) and NaH (24 mg, 1.42 mmol) at −0° C. under N₂ atmosphere and the reaction stirred at room temperature overnight. The mixture was diluted with water (50 mL), extracted with EtOAc (25 mL×3) and the combined organic layers washed with brine, dried over Na₂SO₄ and filtered. The solvent was removed and the residue purified by prep-TLC (eluent: DCM:MeOH=20:1) to afford the tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (300 mg, 70%) as a white solid. LCMS m/z=749.4 [M+H]⁺; ¹H NMR (400 MHz, DMSO-d₆) δ 8.33 (d, J=8.0 Hz, 1H), 7.81 (d, J=12.8 Hz, 1H), 7.35-7.13 (m, 7H), 5.34 (s, 2H), 4.51-4.42 (m, 2H), 4.25-4.18 (m, 1H), 4.06-3.36 (m, 10H), 2.73-2.63 (m, 1H), 2.19-1.67 (m, 9H), 1.56-1.49 (m, 9H), 1.11-0.97 (m, 6H), 0.87-0.82 (m, 1H), 0.68-0.61 (m, 1H).

Step 4: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid To a solution of tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (300 mg, 0.4 mmol) in DCM (5 mL) was added TFA (2 mL). The reaction mixture was stirred at room temperature for 2 h. The mixture was concentrated in vacuo and purified by prep-TLC (eluent: DCM:MeOH=10:1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (238 mg, 86%) as a white solid. LCMS m/z=693.4 [M+H]⁺.

Step 5: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (50 mg, 0.07 mmol) in DMF (1 mL) were added HATU (27 mg, 0.07 mmol) and DIPEA (28 mg, 0.22 mmol). The mixture was stirred at room temperature for 30 min. NH₃·H₂O (1 mL) was added and the reaction stirred at room temperature for another 2 h. The mixture was diluted with water (20 mL), extracted with EtOAc (10 mL×3) and the combined organic layers washed with brine, dried over Na₂SO₄ and filtered. The solvent was removed and the residue purified by prep-TLC (eluent: DCM:MeOH=15:1) to afford the 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (34 mg, 68%) as a white solid. LCMS m/z=692.4 [M+H]⁺; ¹H NMR (400 MHz, DMSO-d₆) δ 8.37 (d, J=11.8 Hz, 1H), 7.85-7.79 (m, 2H), 7.55 (s, 1H), 7.36-7.30 (m, 2H), 7.25-7.14 (m, 5H), 5.34 (s, 2H), 4.49 (s, 2H), 4.33-4.21 (m, 1H), 4.07-4.02 (m, 1H), 3.96-3.38 (m, 9H), 2.82-2.72 (m, 1H), 2.14-1.64 (m, 9H), 1.12-1.02 (m, 6H), 0.87-0.83 (m, 1H), 0.69-0.64 (m, 1H).

Step 6: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N—(N,N-dimethylsulfamoyl)benzamide I′-65 To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (84 mg, 0.12 mmol) in dry THF (2 mL) were added dimethylsulfamoyl chloride (21 mg, 0.14 mmol) and NaH (7 mg, 0.3 mmol) at −0° C. under N₂ atmosphere and the reaction stirred at room temperature overnight. The mixture was diluted with water (50 mL), extracted with EtOAc (20 mL×3) and the combined organic layers washed with brine, dried over Na₂SO₄ and filtered. The solvent was removed and the residue purified by prep-TLC (eluent: DCM:MeOH=15:1) to afford the 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N—(N,N-dimethylsulfamoyl)benzamide (37.9 mg, 39%) as a white solid. LCMS m/z=799.5 [M+H]⁺; ¹H NMR (400 MHz, DMSO-d₆) δ 11.94 (s, 1H), 8.35 (s, 1H), 7.82 (d, J=9 Hz, 1H), 7.36-7.13 (m, 7H), 5.34 (s, 2H), 4.49 (s, 2H), 4.30-4.19 (m, 1H), 4.07-3.84 (m, 3H), 3.83-3.59 (m, 4H), 3.57-3.38 (m, 2H), 3.29 (s, 1H), 2.90 (s, 6H), 2.14 (s, 2H), 1.89-1.69 (m, 5H), 1.38-1.23 (m, 3H), 1.11-1.02 (m, 6H), 0.87-0.85 (m, 1H), 0.68-0.63 (m, 1H).

### 5-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,3,4-oxadiazol-2(3H)-one I′-63

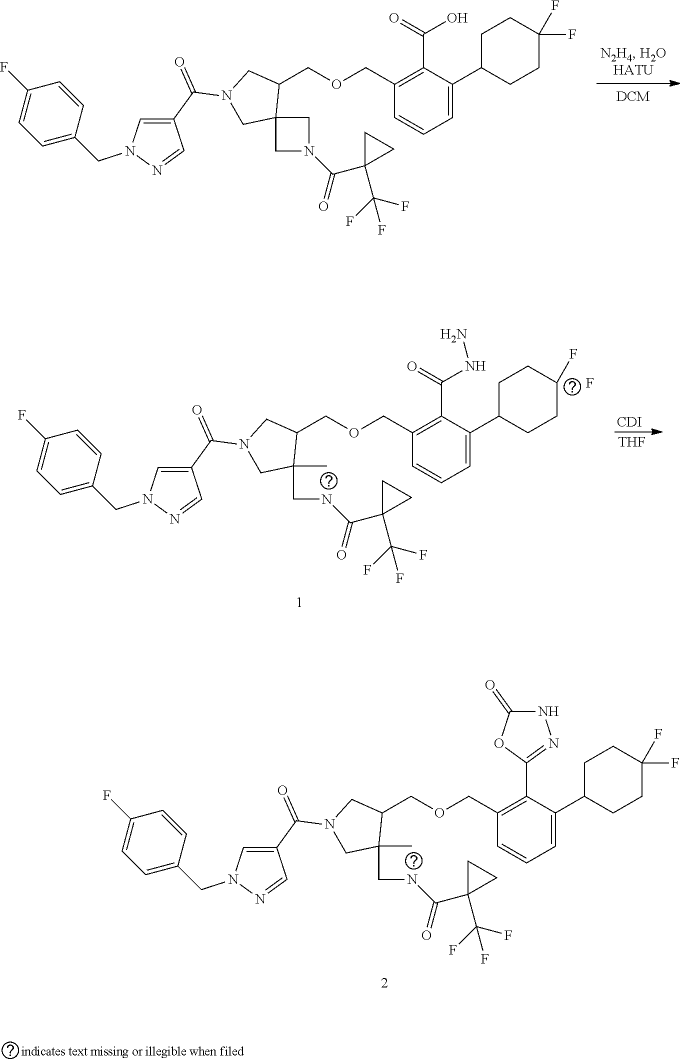

Step 1: 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzohydrazide To a solution of 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (120 mg, 0.164 mmol) in DCM (2 mL) was added HATU (62 mg, 0.164 mmol) and DIPEA (22 mg, 1.64 mmol), after stirring for 15 min. N₂H₄·H₂O (98%, 50 mg, 1 mmol) was added and the mixture was stirred at room temperature for 2 h. The mixture was diluted with water (30 mL) and extracted with EtOAc (50 mL×3). The combined organic layers were washed with brine, dried over Na₂SO₄, filtered and concentrated. The residue obtained was purified by Prep-TLC (DCM:MeOH=10:1) to afford 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzohydrazide (50 mg, 42%) as a white oil. LCMS m/z=747.3 [M+H]⁺; ¹H NMR (400 MHz, DMSO-d₆) δ 9.34 (s, 1H), 8.39-8.33 (m, 1H), 7.82 (d, J=8.6 Hz, 1H), 7.36-7.31 (m, 2H), 7.28-7.14 (m, 5H), 5.34 (s, 2H), 4.53-4.44 (m, 2H), 4.40 (s, 2H), 4.28-3.48 (m, 10H), 2.68-2.61 (m, 1H), 2.17-1.75 (m, 7H), 1.72-1.61 (m, 2H), 1.24 (d, J=5.4 Hz, 4H).

Step 2: 5-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,3,4-oxadiazol-2(3H)-one To a solution of 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzohydrazide (75 mg, 0.1 mmol) in THF (1 mL) was added CDI (41 mg, 0.25 mmol). The reaction mixture was stirred at room temperature for 1 h. The mixture was concentrated and the residue was purified by Prep-HPLC to afford 5-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,3,4-oxadiazol-2(3H)-one (50 mg, 64%) as a white oil. LCMS m/z=773.4 [M+H]⁺; ¹H NMR (400 MHz, Methanol-d₄) δ 8.20 (s, 1H), 7.89 (s, 1H), 7.55-7.39 (m, 2H), 7.38-7.27 (m, 3H), 7.12-7.02 (m, 2H), 5.35 (s, 2H), 4.61-4.50 (m, 3H), 4.26-4.15 (m, 1H), 4.08-3.38 (m, 8H), 2.78-2.66 (m, 1H), 2.64-2.49 (m, 1H), 2.18-2.05 (m, 21), 1.93-1.76 (m, 6H), 1.21-1.04 (m, 4H).

### Allyl 9-(0-((2-oxabicyclo[2.2.2]octan-4-yl)methyl)-L-threonyl)-3,9-diazaspiro undecane-3-carboxylate I′-64

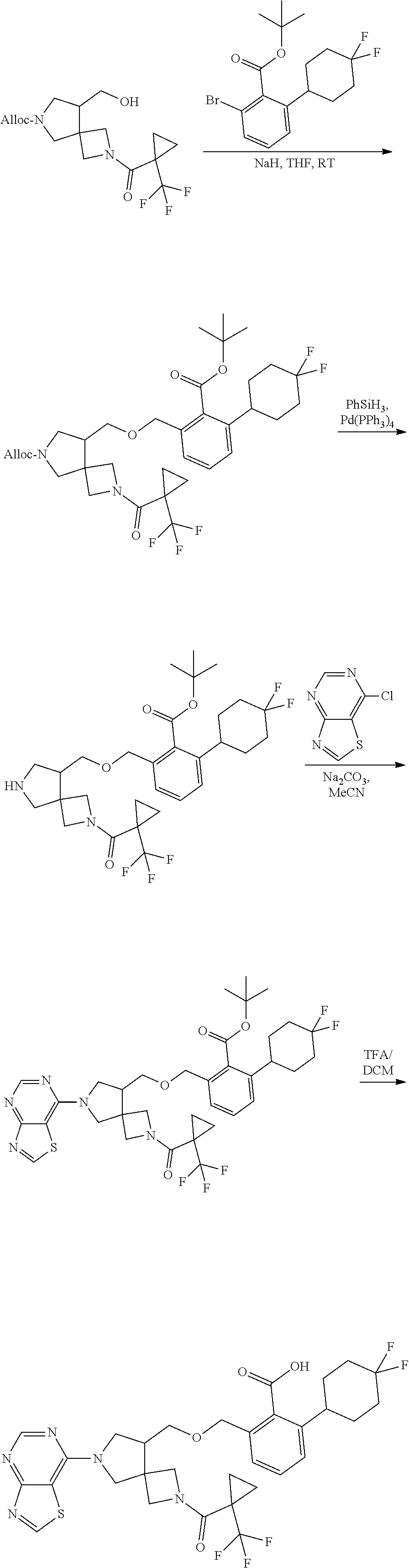

Step 1: allyl 8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate To a solution of allyl 8-(hydroxymethyl)-2-(1-(trifluoromethyl)cyclopropane-1-carbonyl)-2,6-diazaspiro[3.4]octane-6-carboxylate (200 mg, 0.55 mmol) in THF (5 mL) was added tert-butyl 2-bromo-6-(4,4-difluorocyclohexyl)benzoate (215 mg, 0.55 mmol). The reaction mixture was stirred at room temperature overnight. The mixture was diluted with EtOAc (100 mL), washed with water (100 mL×2), dried over Na₂SO₄, filtered and concentrated. The residue obtained was purified by column chromatography on silica gel (eluent: Pet. Ether:EtOAc=20:1 to 1:1) to afford allyl 8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate (250 mg, 66%) as a colorless oil. LCMS m/z=671.3 [M+H]⁺; ¹H NMR (CDCl₃, 400 MHz) δ 7.40-7.30 (m, 1H), 7.24-7.15 (m, 2H), 6.05-5.77 (m, 1H, m), 5.30 (t, J=8.7 Hz, 2H), 5.21 (d, J=10.5 Hz, 1H), 4.66-4.44 (m, 4H), 4.31-3.89 (m, 3H), 3.85-3.44 (m, 5H), 3.36-3.12 (m, 1H), 2.94-2.68 (m, 1H), 2.54-2.34 (m, 1H), 2.31-2.13 (m, 2H), 2.03-1.90 (m, 2H), 1.87-1.73 (m, 4H), 1.59 (s, 9H), 1.29-1.13 (m, 4H).

Step 2: tert-butyl 2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate To a solution of allyl 8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate (250 mg, 0.37 mmol), Pd(PPh₃)₄ (42 mg, 0.037 mmol) in THF (5 mL) was added phenylsilane (200 mg, 1.85 mmol) and the mixture stirred at room temperature for overnight. The solvent was removed under vacuum and purified by prep-TLC (eluent: DCM:MeOH=20:1) to afford tert-butyl 2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate (100 mg, 46%) as a yellow oil. LCMS m/z=587.3 [M+H]⁺; ¹H NMR (CDCl₃, 400 MHz) δ 7.43-7.32 (m, 2H), 7.24-7.14 (m, 1H), 4.56-4.43 (m, 2H), 4.32-4.12 (m, 2H), 4.07-3.71 (m, 2H), 3.61-3.53 (m, 1H), 3.54-3.46 (m, 1H), 3.42-3.28 (m, 1H), 3.22-3.08 (m, 1H), 2.93-2.80 (m, 1H), 2.77-2.59 (m, 1H), 2.43-2.30 (m, 1H), 2.30-2.08 (m, 2H), 2.07-1.81 (m, 6H), 1.60 (s, 9H), 1.21-1.05 (m, 4H).

Step 3: tert-butyl 2-(4,4-difluorocyclohexyl)-6-(((6-(thiazolo[4,5-d]pyrimidin-7-yl)-2-(1-(trifluoromethyl)cyclopropane-1-carbonyl)-2,6-diazaspiro[3.4]octan-8-yl)methoxy)methyl)benzoate To a solution of tert-butyl 2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate (100 mg, 0.16 mmol), 7-chlorothiazolo[4,5-d]pyrimidine (27.7 mg, 0.16 mmol) in MeCN (5 mL) was added Na₂CO₃ (53 mg, 0.48 mmol). The reaction mixture was stirred at room temperature overnight. The mixture was diluted with EtOAc (100 mL), washed with water (100 mL×2), dried over Na₂SO₄, filtered and concentrated. The residue obtained was purified by prep-TLC (eluent: MeOH:DCM=1:10) to afford tert-butyl 2-(4,4-difluorocyclohexyl)-6-(((6-(thiazolo[4,5-d]pyrimidin-7-yl)-2-(1-(trifluoromethyl)cyclopropane-1-carbonyl)-2,6-diazaspiro[3.4]octan-8-yl)methoxy)methyl)benzoate (75 mg, 61%) as a white solid. LCMS m/z=722.4 [M+H]⁺; ¹H NMR (CDCl₃, 400 MHz) δ 9.26 (s, 1H), 8.64 (s, 1H), 7.33-7.27 (m, 1H), 7.26-7.22 (m, 1H), 7.17 (d, J=7.5 Hz, 1H), 4.54 (s, 2H), 4.38-3.91 (m, 6H), 3.87-3.70 (m, 1H), 3.69-3.52 (m, 2H), 2.86-2.55 (m, 2H), 2.30-2.08 (m, 2H), 2.04-1.89 (m, 2H), 1.88-1.71 (m, 5H), 1.57 (s, 9H), 1.27-1.10 (m, 4H).

Step 4: allyl 9-(O-((2-oxabicyclo[2.2.2]octan-4-yl)methyl)-L-threonyl)-3,9-diazaspiro[5.5]undecane-3-carboxylate I′-64 To a solution of tert-butyl 2-(4,4-difluorocyclohexyl)-6-(((6-(thiazolo[4,5-d]pyrimidin-7-yl)-2-(1-(trifluoromethyl)cyclopropane-1-carbonyl)-2,6-diazaspiro[3.4]octan-8-yl)methoxy)methyl)benzoate (70 mg, 0.097 mmol) in DCM (2 mL) was added TFA (1 mL). The reaction mixture was stirred at room temperature for 2 h. The solvent was removed under vacuum and purified by prep-HPLC to afford 2-(4,4-difluorocyclohexyl)-6-(((6-(thiazolo[4,5-d]pyrimidin-7-yl)-2-(1-(trifluoromethyl)cyclopropane-1-carbonyl)-2,6-diazaspiro[3.4]octan-8-yl)methoxy)methyl)benzoic acid (35 mg, 54%) as a white solid. LCMS m/z=666.4 [M+H]⁺; ¹H NMR (CDCl₃, 400 MHz) δ 7.40-7.30 (m, 1H), 7.24-7.15 (m, 2H), 6.05-5.77 (m, 1H), 5.30 (t, J=8.7 Hz, 2H), 5.21 (d, J=10.5 Hz, 1H), 4.66-4.44 (m, 4H), 4.31-3.89 (m, 3H), 3.85-3.44 (m, 5H), 3.36-3.12 (m, 1H), 2.94-2.68 (m, 1H), 2.54-2.34 (m, 1H), 2.31-2.13 (m, 2H), 2.03-1.90 (m, 2H), 1.87-1.73 (m, 4H), 1.59 (s, 9H), 1.29-1.13 (m, 4H).

### 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid I-128

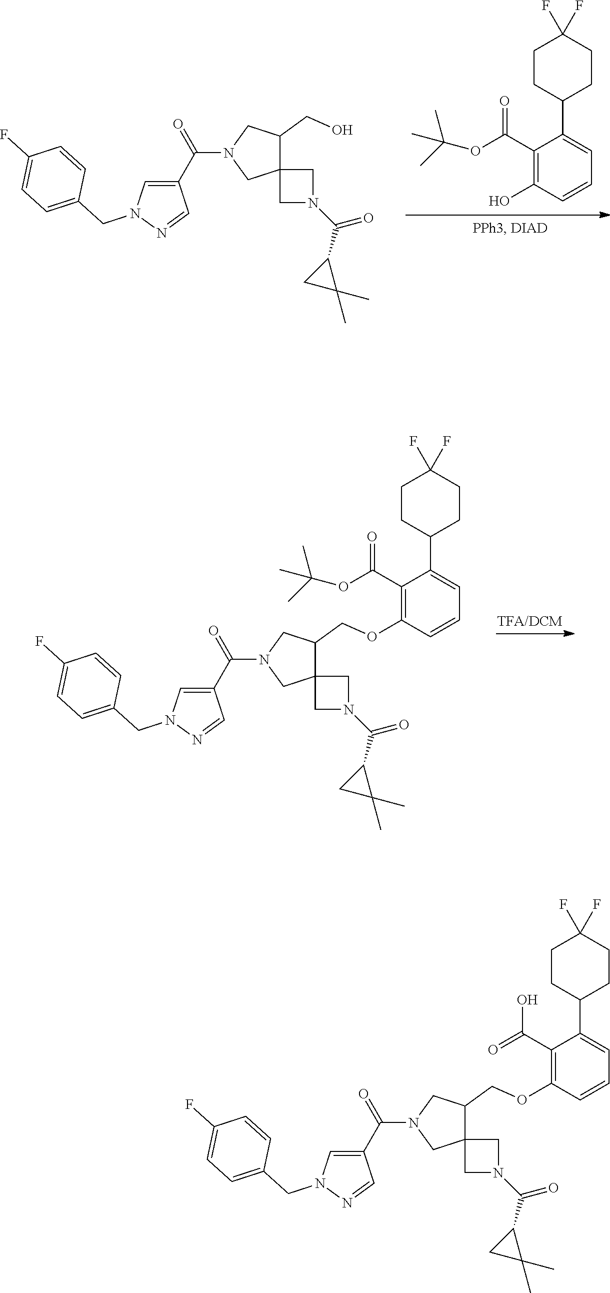 [SMILES: CC1(C)C[C@@H]1C(=O)N1CC2(CN(C(=O)c3cnn(Cc4ccc(F)cc4)c3)CC2CO)C1 ; CC(C)(C)OC(=O)c1c(O)cccc1C1CCC(F)(F)CC1 ; CC(C)(C)OC(=O)c1c(OCC2CN(C(=O)c3cnn(Cc4ccc(F)cc4)c3)CC23CN(C(=O)[C@H]2CC2(C)C)C3)cccc1C1CCC(F)(F)CC1 ; CC1(C)C[C@@H]1C(=O)N1CC2(CN(C(=O)c3cnn(Cc4ccc(F)cc4)c3)CC2COc2cccc(C3CCC(F)(F)CC3)c2C(=O)O)C1]

Step 1: tert-butyl 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate To a solution of tert-butyl 2-(4,4-difluorocyclohexyl)-6-hydroxybenzoate (100 mg, 0.32 mmol) in Toluene (2 mL) was added (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (141 mg, 0.32 mmol), PPh₃ (168 mg, 0.64 mmol) and DIAD (130 mg, 0.64 mmol). The reaction mixture was stirred at 130° C. for 1 h in microwave. The mixture was concentrated and purified by prep-HPLC to afford tert-butyl 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate (60 mg, 25%) as a white solid. LCMS m/z=735.4 [M+H]⁺; ¹H NMR (400 MHz, Chloroform-d) δ 7.94-7.79 (m, 2H), 7.34-7.29 (m, 2H), 7.10-7.00 (m, 2H), 6.96-6.81 (m, 2H), 6.79-6.72 (m, 1H), 5.28 (s, 2H), 4.30-3.70 (m, 11H), 1.98-1.89 (m, 4H), 1.86-1.74 (m, 7H), 1.65 (s, 9H), 1.37-1.23 (m, 7H).

Step 2: 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid I-128 To a solution of tert-butyl 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate (60 mg, 0.08 mmol) in DCM (2 mL) was added TFA (1 mL). The reaction mixture was stirred at room temperature for 2 h. The mixture was concentrated and purified by prep-HPLC to afford 2-(4,4-difluorocyclohexyl)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid (I-128, 26.4 mg, 48%) as a white solid. LCMS m/z=679.5 [M+H]⁺; ¹H NMR (400 MHz, Chloroform-d) δ 7.95-7.78 (m, 2H), 7.34-7.27 (m, 2H), 7.24-7.21 (m, 1H), 7.08-6.99 (m, 2H), 6.97-6.90 (m, 1H), 6.84-6.68 (m, 1H), 5.27 (s, 2H), 4.43-3.83 (m, 11H), 2.72-2.66 (m, 1H), 2.19-2.11 (m, 2H), 1.96-1.76 (m, 6H), 1.37-1.12 (m, 7H), 1.11-1.04 (m, 2H).

TABLE 17
The compounds listed in Table 17 were synthesized from tert-butyl 2-bromo-
6-(((6-(1-(4-fluorobenzyl)-1H-pyrazole-4-carbonyl)-2-(1-(trifluoromethyl)cyclopropane-1-
carbonyl)-2,6-diazaspiro[3.4]octan-8-yl)methoxy)methyl)benzoate according to the procedures
outlined for using the appropriate commercially available reagents and/or intermediates described
elsewhere.

| Cmpd. | | | |
|---|---|---|---|
| No. | Compound | ¹HNMR | LCMS |
| I-130 | 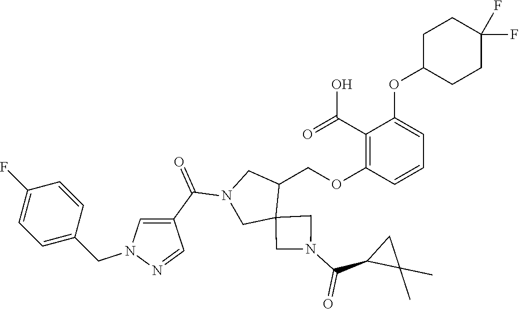 [SMILES: CC1(C)C[C@@H]1C(=O)N1CC2(CN(C(=O)c3cnn(Cc4ccc(F)cc4)c3)CC2COc2cccc(OC3CCC(F)(F)CC3)c2C(=O)O)C1] | ¹H NMR (400 MHz, Methanol-d₄) δ 8.26 (d, J = 10.2 Hz, 1H), 7.95 (s, 1H), 7.33 (s, 3H), 7.07 (s, 2H), 6.73 (d, J = 6.0 Hz, 2H), 5.36 (d, J = 6.8 Hz, 2H), 4.56-4.42 (m, 2H), 4.36-3.82 (m, 9H), 2.96- 2.77 (m, 1H), 2.16 (d, J = 13.8 Hz, 2H), 2.07- 1.97 (m, 2H), 1.87 (s, 4H), 1.64-1.37 (m, 1H), 1.22-0.97 (m, 7H), 0.76 (s, 1H). | m/z = 695.3 [M + H]⁺ |

### (S)-2-((4-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid I′-57

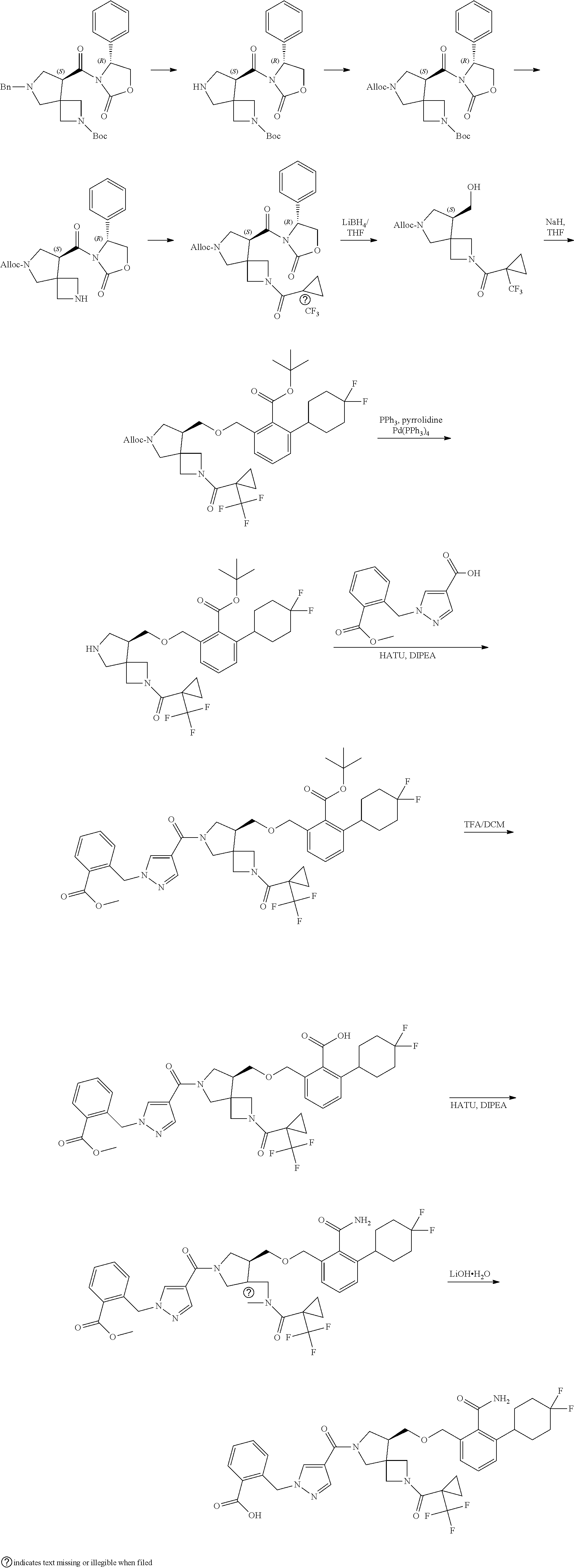

Step 1: tert-butyl (S)-8-((R)-2-oxo-4-phenyloxazolidine-3-carbonyl)-2,6-diazaspiro[3.4]octane-2-carboxylate To a solution of tert-butyl (S)-6-benzyl-8-((R)-2-oxo-4-phenyloxazolidine-3-carbonyl)-2,6-diazaspiro[3.4]octane-2-carboxylate (1.7 g, 3.45 mmol) in EtOAc (13 mL) was added Pd/C (850 mg). The reaction mixture was stirred at 50° C. for 36 h with H₂ balloon. The mixture was filtrated with MeOH and concentrated under vacuum to afford tert-butyl (S)-8-((R)-2-oxo-4-phenyloxazolidine-3-carbonyl)-2,6-diazaspiro[3.4]octane-2-carboxylate (1.1 g, 86%) as a white solid. LCMS m/z=402.2 [M+H]⁺.

Step 2: 6-allyl 2-(tert-butyl) (S)-8-((R)-2-oxo-4-phenyloxazolidine-3-carbonyl)-2,6-diazaspiro[3.4]octane-2,6-dicarboxylate To a solution of tert-butyl (S)-8-((R)-2-oxo-4-phenyloxazolidine-3-carbonyl)-2,6-diazaspiro[3.4]octane-2-carboxylate (3.5 g, 8.7 mmol) in DCM 40 mL) was added AllocCl (1 g, 8.7 mmol) and TEA (2.6 g, 25.7 mmol). The reaction mixture was stirred at room temperature overnight. The mixture was diluted with water (50 mL) and extracted with DCM (50 mL×3). The combined organic layers were purified by chromatography on silica gel (eluent: PE:EtOAc=1:1) to afford 6-allyl 2-(tert-butyl) (S)-8-((R)-2-oxo-4-phenyloxazolidine-3-carbonyl)-2,6-diazaspiro[3.4]octane-2,6-dicarboxylate as a colorless oil. ¹H NMR (400 MHz, DMSO-d₆) δ 7.42-7.38 (m, 2H), 7.35-7.33 (m, 3H), 5.94-5.79 (m, 1H), 5.47-5.41 (m, 1H), 5.24-5.09 (m, 2H), 4.96-4.89 (m, 1H), 4.78-4.65 (m, 2H), 4.53-4.39 (m, 2H), 4.28-4.13 (m, 2H), 3.99-3.92 (m, 1H), 3.71-3.52 (m, 4H), 3.47-3.37 (m, 1H), 1.37 (s, 9H).

Step 3: allyl (S)-8-((R)-2-oxo-4-phenyloxazolidine-3-carbonyl)-2,6-diazaspiro[3.4]octane-6-carboxylate: To a solution of 6-allyl 2-(tert-butyl) (S)-8-((R)-2-oxo-4-phenyloxazolidine-3-carbonyl)-2,6-diazaspiro[3.4]octane-2,6-dicarboxylate (2.1 g, 4.3 mmol) in a mixture of TFA and DCM (10 mL/20 mL) was stirred at RT for 1 h. The reaction was concentrated to afford allyl (S)-8-((R)-2-oxo-4-phenyloxazolidine-3-carbonyl)-2,6-diazaspiro[3.4]octane-6-carboxylate (1.7 g) as a yellow oil and used directly at next step. LCMS m/z=386.1 [M+H]⁺.

Step 4: allyl (S)-8-((R)-2-oxo-4-phenyloxazolidine-3-carbonyl)-2-(1-(trifluoromethyl)cyclopropane-1-carbonyl)-2,6-diazaspiro[3.4]octane-6-carboxylate To a solution of allyl (S)-8-((R)-2-oxo-4-phenyloxazolidine-3-carbonyl)-2,6-diazaspiro[3.4]octane-6-carboxylate (1.7 g, 4.3 mmol) in DCM (30 mL) was added 1-((difluoro-13-methyl)-12-fluoranyl)cyclopropane-1-carboxylic acid (667 mg, 4.3 mmol), DIEA (1.67 g, 12.9 mmol) and HATU (1.67 g, 4.3 mmol). The reaction mixture was stirred at RT for 2 h. The mixture was washed with 1 M HCl (300 mL) and washed with brine, dried over Na₂SO₄, filtered and concentrated and purified by RP-column to afford allyl (S)-8-((R)-2-oxo-4-phenyloxazolidine-3-carbonyl)-2-(1-(trifluoromethyl)cyclopropane-1-carbonyl)-2,6-diazaspiro[3.4]octane-6-carboxylate (1.8 g, 80%) as a yellow solid. LCMS m/z=522.2 [M+H]⁺, ¹H NMR (400 MHz, DMSO-d₆) δ 7.36-7.23 (m, 5H), 5.97-5.80 (m, 1H), 5.46 (d, J=8.9, 4.2 Hz, 1H), 5.25-5.10 (m, 2H), 4.76 (t, 1H), 4.51-4.44 (m, 2H), 4.36-4.14 (m, 4H), 3.90 (s, 1H), 3.77-3.57 (m, 4H), 3.44 (m, 1H), 1.24 (s, 4H).

Step 5: allyl (S)-8-(hydroxymethyl)-2-(1-(trifluoromethyl)cyclopropane-1-carbonyl)-2,6-diazaspiro[3.4]octane-6-carboxylate To a solution of allyl (S)-8-((R)-2-oxo-4-phenyloxazolidine-3-carbonyl)-2-(1-(trifluoromethyl)cyclopropane-1-carbonyl)-2,6-diazaspiro[3.4]octane-6-carboxylate (1.85 g, 3.55 mmol) in THF (30 mL) was added dropwise LiBH₄ (1.24 mL, 2.48 mmol) at −78° C. under N₂, the reaction mixture was stirred at room temperature overnight. The mixture was concentrated and purified by RP column (38% ACN in water) to afford allyl (S)-8-(hydroxymethyl)-2-(1-(trifluoromethyl)cyclopropane-1-carbonyl)-2,6-diazaspiro[3.4]octane-6-carboxylate (370 mg, 29%) as a yellow oil. LCMS m/z=363.2 [M+H]⁺; ¹H NMR (400 MHz, DMSO-d₆) δ 5.97-5.87 (m, 1H), 5.28 (d, J=17.2 Hz, 1H), 5.18 (d, J=10.4, 1.6 Hz, 1H), 4.78 (t, J=4.8 Hz, 1H), 4.50 (d, J=5.4, 1.6 Hz, 2H), 4.37-4.06 (m, 2H), 3.99-3.62 (m, 2H), 3.60-3.49 (m, 3H), 3.47-3.36 (m, 2H), 3.25-3.15 (m, 1H), 2.38-2.26 (m, 1H), 1.27-1.20 (m, 2H), 1.17-1.12 (m, 2H).

Step 6: allyl (S)-8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate To a solution of allyl (S)-8-(hydroxymethyl)-2-(1-(trifluoromethyl)cyclopropane-1-carbonyl)-2,6-diazaspiro[3.4]octane-6-carboxylate (450 mg, 1.24 mmol) in THE (20 mL) was added tert-butyl 2-(bromomethyl)-6-(4,4-difluorocyclohexyl)benzoate (484 mg, 1.24 mmol) and NaH (300 mg, 7.45 mmol), the reaction mixture was stirred at room temperature for 2 h. The mixture was diluted with water (20 mL) and extracted with EtOAc (30 mL×3). The combined organic layers were washed with brine, dried over Na₂SO₄, filtered and concentrated. The residue obtained was purified by prep-TLC (eluent: DCM:MeOH=20:1) to afford allyl (S)-8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate (700 mg, 84%) as a yellow oil. LCMS m/z=671.3 [M+H]⁺; ¹H NMR (400 MHz, DMSO-d₆) δ 7.40-7.30 (m, 2H), 7.25 (d, J=7.4 Hz, 1H), 5.96-5.85 (m, 1H), 5.27 (d, J=17.4 Hz, 1H), 5.17 (d, J=10.4 Hz, 1H), 4.50 (m, 2H), 4.46 (s, 2H), 4.40-3.64 (m, 4H), 3.61-3.37 (m, 6H), 3.23-3.14 (m, 1H), 2.68 (t, J=12.0 Hz, 1H), 2.19-2.09 (m, 2H), 2.03-1.80 (m, 4H), 1.79-1.67 (m, 2H), 1.55 (s, 9H), 1.27-1.21 (m, 2H), 1.12-1.07 (m, 2H).

Step 7: tert-butyl (S)-2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate To a solution of allyl (S)-8-(((2-(tert-butoxycarbonyl)-3-(4,4-difluorocyclohexyl)benzyl)oxy)methyl)-2-(1-(trifluoromethyl)cyclopropane-1-carbonyl)-2,6-diazaspiro[3.4]octane-6-carboxylate (550 mg, 0.82 mmol) in DCM (15 mL) was added PPh₃ (54 mg, 0.21 mmol) and pyrrolidine (0.41 mL, 4.92 mmol), the reaction mixture was stirred at room temperature for 10 mins, then Pd(PPh₃)₄ (95 mg, 0.08 mmol) was added, the reaction mixture was stirred at room temperature for 1 h. The mixture was diluted with water (1 mL) and extracted with DCM (20 mL×3). The combined organic layers were washed with brine, dried over Na₂SO₄, filtered and concentrated. The residue obtained was purified by prep-TLC (eluent: DCM:MeOH=12:1) to afford tert-butyl (S)-2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate (320 mg, 66%) as a yellow oil. LCMS m/z=587.3 [M+H]⁺; ¹H NMR (400 MHz, DMSO-d₆) δ 7.38-7.30 (m, 2H), 7.25 (d, J=7.2 Hz, 1H), 4.44 (s, 2H), 4.35-3.51 (m, 5H), 3.49-3.40 (m, 2H), 2.98-2.83 (m, 3H), 2.69 (t, J=11.8 Hz, 1H), 2.49-2.43 (m, 1H), 2.27-2.20 (m, 1H), 2.19-2.11 (m, 2H), 1.93-1.81 (m, 4H), 1.73 (t, J=12.4 Hz, 2H), 1.56 (s, 9H), 1.26-1.22 (m, 2H), 1.11-1.08 (m, 2H).

Step 8: tert-butyl (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate To a solution of 1-(2-(methoxycarbonyl)benzyl)-1H-pyrazole-4-carboxylic acid (62 mg, 0.24 mmol) in DCM (4 mL) was added HATU (100 mg, 0.26 mmol) and the mixture was stirred at room temperature for 30 min. tert-butyl (S)-2-(4,4-difluorocyclohexyl)-6-(((2-(1-(trifluoromethyl)cyclopropane-1-carbonyl)-2,6-diazaspiro[3.4]octan-8-yl)methoxy)methyl)benzoate (140 mg, 0.24 mmol) and DIPEA (124 mg, 0.95 mmol) were added and the reaction stirred at room temperature for another 2 h. The mixture was diluted with water (20 mL) and extracted with DCM (10 mL×3). The combined organic layers were washed with brine, dried over Na₂SO₄, filtered and concentrated. The residue obtained was purified by prep-TLC (eluent: DCM:MeOH=20:1) to afford tert-butyl (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (205 mg, 80%) as a colorless oil. LCMS m/z=829.3 [M+H]⁺; ¹H NMR (400 MHz, DMSO-d₆) δ 8.29 (d, J=4.6 Hz, 1H), 7.88 (dd, J=27.8, 9.8 Hz, 2H), 7.53 (t, J=7.2 Hz, 1H), 7.45 (t, J=7.6 Hz, 1H), 7.35-7.21 (m, 3H), 6.82 (d, J=7.8 Hz, 1H), 5.72 (s, 2H), 4.46 (d, J=7.2 Hz, 2H), 4.27-4.03 (m, 2H), 4.02-3.88 (m, 2H), 3.86 (d, J=6.2 Hz, 3H), 3.82-3.67 (m, 2H), 3.64-3.34 (m, 5H), 2.66-2.58 (m, 1H), 2.19-2.08 (m, 2H), 1.90-1.65 (m, 6H), 1.53 (d, J=14.2 Hz, 9H), 1.24-1.22 (m, 2H), 1.13-1.10 (m, 2H).

Step 9: (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid To a solution of tert-butyl (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (160 mg, 0.19 mmol) in DCM (4 mL) was added TFA (2 mL). The reaction mixture was stirred at room temperature for 2 h. The solvent was removed under vacuum and purified by prep-TLC (eluent: DCM:MeOH=10:1) to afford (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (150 mg, 80%) as a yellow solid. LCMS m/z=773.3 [M+H]⁺; ¹H NMR (400 MHz, DMSO-d₆) δ 8.33 (d, J=18.4 Hz, 1H), 7.89 (dd, J=23.2, 9.2 Hz, 2H), 7.54 (q, J=7.4 Hz, 1H), 7.45 (t, J=6.4 Hz, 1H), 7.27-7.15 (m, 3H), 6.82 (d, J=7.8 Hz, 1H), 5.72 (s, 2H), 4.49 (s, 2H), 4.30-4.06 (m, 2H), 4.03-3.91 (m, 2H), 3.86 (d, J=5.8 Hz, 3H), 3.82-3.68 (m, 2H), 3.65-3.45 (m, 5H), 2.79 (t, J=12.0 Hz, 1H), 2.13-2.03 (m, 2H), 1.91-1.61 (m, 6H), 1.24-1.22 (m, 4H).

Step 10: methyl (S)-2-((4-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate To a solution of (S)-2-(4,4-difluorocyclohexyl)-6-(((6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (130 mg, 0.17 mmol) in DMF (3 mL) was added HATU (64 mg, 0.17 mmol) and DIPEA (65 mg, 0.5 mmol), the mixture was stirred at room temperature for 30 min. NH₃·H₂O (2.5 mL, 0.17 mmol) was added and the reaction stirred at room temperature for another 2 h. The mixture was diluted with water (20 mL) and extracted with EtOAc (10 mL×3). The combined organic layers were washed with brine, dried over Na₂SO₄, filtered and concentrated. The residue obtained was purified by prep-TLC (eluent: DCM:MeOH=10:1) to afford methyl (S)-2-((4-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (68 mg, 52%) as a yellow solid. LCMS m/z=772.3 [M+H]⁺; ¹H NMR (400 MHz, DMSO-d₆) δ 8.31 (d, J=6.0 Hz, 1H), 7.94-7.81 (m, 3H), 7.58-7.51 (m, 2H), 7.45 (d, J=7.8 Hz, 1H), 7.27-7.19 (m, 3H), 6.83 (d, J=7.8 Hz, 1H), 5.72 (s, 2H), 4.49 (s, 2H), 4.32-4.06 (m, 2H), 3.97 (q, J=8.6 Hz, 2H), 3.86 (m, 3H), 3.83-3.63 (m, 3H), 3.59-3.34 (m, 3H), 2.76 (t, J=12.8 Hz, 1H), 2.68-2.53 (m, 1H), 2.15-2.04 (m, 2H), 1.90-1.66 (m, 6H), 1.24-1.22 (m, 4H).

Step 11: (S)-2-((4-(8-(((2-carbamoyl-3-(4,4-difluorocyclohexyl)benzyl)oxy) methyl)-2-(1-(trifluoromethyl)cyclopropane-1-carbonyl)-2,6-diazaspiro[3.4]octane-6-carbonyl)-1H-pyrazol-1-yl)methyl)benzoic acid I′-57 To a solution of methyl (S)-2-((4-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (60 mg, 0.08 mmol) in a mixture of THF, water and MeOH (2 mL/0.5 mL/0.5 mL) was added LiOH (10 mg, 0.23 mmol). The reaction mixture was stirred at room temperature for 2 h, acidified to pH˜2 with 1 M HCl and concentrated, purified by prep-HPLC to afford (S)-2-((4-(8-(((2-carbamoyl-3-(4,4-difluorocyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoic acid (I′-57, 35.1 mg, 59%) as a white solid. LCMS m/z=758.4 [M+H]⁺; ¹H NMR (400 MHz, DMSO-d₆) δ 8.31 (d, J=4.8 Hz, 1H), 7.94-7.80 (m, 3H), 7.57-7.36 (m, 3H), 7.28-7.19 (m, 3H), 6.77 (t, J=6.8 Hz, 1H), 5.75 (d, J=4.0 Hz, 2H), 4.49 (s, 2H), 4.32-4.04 (m, 2H), 3.99-3.39 (m, 8H), 2.77 (t, J=12.8 Hz, 1H), 2.65-2.53 (m, 1H), 2.15-2.07 (m, 2H), 1.91-1.66 (m, 6H), 1.22-1.10 (m, 4H).

### (S)-(8-(((3-(4,4-difluorocyclohexyl)-2-(2H-tetrazol-5-yl)benzyl)oxy)methyl)-2-(1-(trifluoromethyl)cyclopropane-1-carbonyl)-2,6-diazaspiro[3.4]octan-6-yl)(1-(4-fluorobenzyl)-1H-pyrazol-4-yl)methanone I′-54

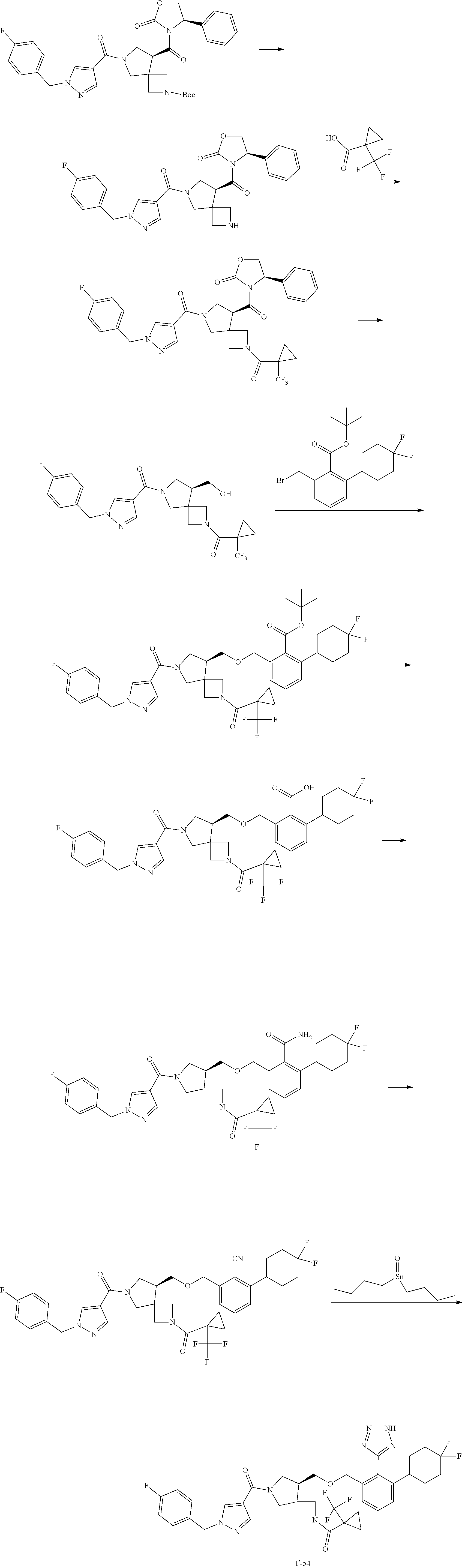

Step 1: (R)-3-((S)-6-(1-(4-fluorobenzyl)-1H-pyrazole-4-carbonyl)-2,6-diazaspiro[3.4]octane-8-carbonyl)-4-phenyloxazolidin-2-one: To a solution of tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate (140 mg, 0.23 mmol) in DCM (2 mL) was added TFA (0.5 mL). The reaction mixture was stirred at room temperature for 2 h. The solvent was removed under vacuum to afford crude (R)-3-((S)-6-(1-(4-fluorobenzyl)-1H-pyrazole-4-carbonyl)-2,6-diazaspiro[3.4]octane-8-carbonyl)-4-phenyloxazolidin-2-one (117 mg, 100%) which was used directly in the next step. LCMS m/z=504.2 [M+H]⁺.

Step 2: (R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one: To a solution of 1-(trifluoromethyl)cyclopropane-1-carboxylic acid (1 g, 7.09 mmol) in DCM (10 mL) was added HATU (2.69 g, 7.09 mmol) and DIPEA (2.75 g, 21.27 mmol). The mixture was stirred at room temperature for 30 min. (R)-3-((S)-6-(1-(4-fluorobenzyl)-1H-pyrazole-4-carbonyl)-2,6-diazaspiro[3.4]octane-8-carbonyl)-4-phenyloxazolidin-2-one (3.57 g, 7.09 mmol) was added. The reaction was stirred at room temperature for another 4 h. The mixture was diluted with water (100 mL) and extracted with DCM (100 mL×3). The combined organic layers were washed with brine, dried over Na₂SO₄, filtered and concentrated. The residue obtained was purified by column chromatography on silica gel (eluent: DCM:MeOH=50:1 to 15:1) to afford (R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (2.2 g, 48% yield) as a yellow oil. LCMS m/z=640.2 [M+H]; ¹H NMR (400 MHz, DMSO-d₆) δ 8.34-8.27 (m, 1H), 7.82-7.74 (m, 1H), 7.37-7.23 (m, 5H), 7.22-7.04 (m, 4H), 5.49-5.42 (m, 1H), 5.39-5.28 (m, 2H), 4.80-4.71 (m, 1H), 4.42-4.07 (m, 6H), 3.94-3.75 (m, 4H), 1.25-1.23 (m, 2H), 1.17-1.13 (m, 2H).

Step 3: (S)-(1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone: To a solution of (R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (500 mg, 0.76 mmol) in THF (4 mL) was added LiBH₄ (0.23 mL, 0.47 mmol) at 0° C. The reaction mixture was stirred at 0° C. under a N₂ atmosphere for overnight. The mixture was diluted with water (100 mL) and extracted with EtOAc (200 mL×2). The combined organic layers were washed with brine, dried over Na₂SO₄, filtered and concentrated. The residue obtained was purified by prep-TLC (eluent: DCM:MeOH=10:1, v/v) to afford (S)-(1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (170 mg, 45% yiled) as a white solid. LCMS m/z=481.2 [M+H]⁺; ¹H NMR (400 MHz, DMSO-d₆) δ 8.35-8.30 (m, 111), 7.84-7.77 (m, 1H), 7.36-7.19 (m, 4H), 5.36-5.30 (m, 2H), 4.97-4.75 (m, 1H), 4.54-4.12 (m, 2H), 4.04-3.41 (m, 8H), 1.25-1.22 (m, 2H), 1.18-1.13 (m, 2H).

Step 4: tert-butyl (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate: To a solution of (S)-(1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (110 mg, 0.23 mmol) and tert-butyl 2-(bromomethyl)-6-(4,4-difluorocyclohexyl)benzoate (89 mg, 0.23 mmol) in THE (2 mL). The mixture was stirred at room temperature for overnight. The mixture was diluted with water (50 mL) and extracted with EtOAc (100 mL×2). The combined organic layers were washed with brine, dried over Na₂SO₄, filtered and concentrated. The residue obtained was purified by prep-TLC (eluent: DCM:MeOH=20:1, v/v) to afford tert-butyl (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (160 mg, 89% yield) as a white solid. LCMS m/z=789.3 [M+H]⁺; ¹H NMR (400 MHz, DMSO-d₆) δ 8.32 (d, J=3.8 Hz, 1H), 7.86-7.78 (m, 1H), 7.34-7.30 (m, 3H), 7.27-7.22 (m, 2H), 7.20-7.14 (m, 2H), 5.35-5.32 (m, 2H), 4.52-4.44 (m, 2H), 4.31-4.13 (m, 1H), 4.03-3.36 (m, 7H), 3.30-3.18 (m, 2H), 2.71-2.61 (m, 1H), 2.18-2.08 (m, 2H), 1.92-1.63 (m, 7H), 1.54 (s, 3H), 1.52-1.48 (m, 6H), 1.18-1.09 (m, 4H).

Step 5: (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid: To a solution of tert-butyl (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (160 mg, 0.20 mmol) in DCM (2 mL) was added TFA (1 mL). The reaction mixture was stirred at room temperature for 2 h. The solvent was removed under vacuum. The residue was purified by prep-HPLC to afford (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (5 mg, 8%) as a white solid. LCMS m/z=733.3 [M+H]⁺; ¹H NMR (400 MHz, DMSO-d₆) δ 8.36-8.31 (m, 1H), 7.82 (d, J=9.0 Hz, 1H), 7.36-7.28 (m, 4H), 7.27-7.12 (m, 3H), 5.36-5.31 (m, 2H), 4.55-4.45 (m, 2H), 4.42-3.45 (m, 10H), 3.41-3.35 (m, 1H), 2.81-2.71 (m, 1H), 2.65-2.54 (m, 1H), 2.17-2.03 (m, 2H), 1.90-1.79 (m, 3H), 1.76-1.64 (m, 2H), 1.27-1.07 (m, 4H).

Step 6: (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide: To a solution of (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (50 mg, 0.07 mol) in DCM (2 mL) was added HATU (26 mg, 0.07 mmol) and DIPEA (26 mg, 0.21 mmol). The mixture was stirred at room temperature for 30 min. NH·H₂O (1 mL) was added. The reaction was stirred at room temperature for another 2 h. The mixture was diluted with water (50 mL) and extracted with DCM (50 mL×3). The combined organic layers were washed with brine, dried over Na₂SO₄, filtered and concentrated. The residue obtained was purified by prep-TLC (eluent: DCM:MeOH=10:1, v/v) to afford (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide (41 mg, 83%) as a yellow oil. LCMS m/z=732.3 [M+H]⁺; ¹H NMR (400 MHz, DMSO-d₆) δ 8.40-8.31 (m, 1H), 7.88-7.78 (m, 2H), 7.55 (s, 1H), 7.38-7.30 (m, 2H), 7.27-7.10 (m, 5H), 5.34 (s, 2H), 4.49 (s, 2H), 4.00-3.52 (m, 8H), 2.82-2.72 (m, 1H), 2.68-2.53 (m, 1H), 2.16-2.04 (m, 2H), 1.93-1.81 (m, 3H), 1.75-1.62 (m, 2H), 1.23 (s, 4H), 1.14 (s, 2H).

Step 7: (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile: To a solution of (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (40 mg, 0.055 mol) in DCM (2 mL) was added TEA (17 mg, 0.165 mmol) and TFAA (17 mg, 0.083 mmol). The mixture was stirred at room temperature for 2 h. The mixture was diluted with water (50 mL) and extracted with DCM (50 mL×3). The combined organic layers were washed with brine, dried over Na₂SO₄, filtered and concentrated. The residue obtained was purified by prep-TLC (eluent: DCM:MeOH=10:1, v/v) to afford (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile (35 mg, 89%) as a colorless oil. LCMS m/z=714.3 [M+H]⁺.

Step 8: (S)-(8-(((3-(4,4-difluorocyclohexyl)-2-(2H-tetrazol-5-yl)benzyl)oxy)methyl)-2-(1-(trifluoromethyl)cyclopropane-1-carbonyl)-2,6-diazaspiro[3.4]octan-6-yl)(1-(4-fluorobenzyl)-1H-pyrazol-4-yl)methanone I′-57: To a solution of (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile (40 mg, 0.07 mol) in toluene (1 mL) was added dibutylstannanone (7 mg, 0.03 mmol) and TMSN₃ (24 mg, 0.21 mmol). The mixture was stirred in sealed tube at 150° C. for overnight. The mixture was diluted with water (50 mL) and extracted with EtOAc (50 mL×3). The combined organic layers were washed with brine, dried over Na₂SO₄, filtered and concentrated. The residue obtained was purified by prep-HPLC to afford (S)-(8-(((3-(4,4-difluorocyclohexyl)-2-(2H-tetrazol-5-yl)benzyl)oxy)methyl)-2-(1-(trifluoromethyl)cyclopropane-1-carbonyl)-2,6-diazaspiro[3.4]octan-6-yl)(1-(4-fluorobenzyl)-1H-pyrazol-4-yl)methanone (4.3 mg, 8.1%) as a white solid. LCMS m/z=757.4 [M+H]; ¹H NMR (400 MHz, DMSO-d₆) δ 8.48-8.32 (m, 1H), 8.13 (s, 1H), 7.81 (d, J=8.9 Hz, 1H), 7.37-7.28 (m, 4H), 7.21-7.12 (m, 2H), 5.37 (d, J=15.8 Hz, 2H), 4.23-3.37 (m, 13H), 2.17-1.08 (m, 13H).

### (S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-58

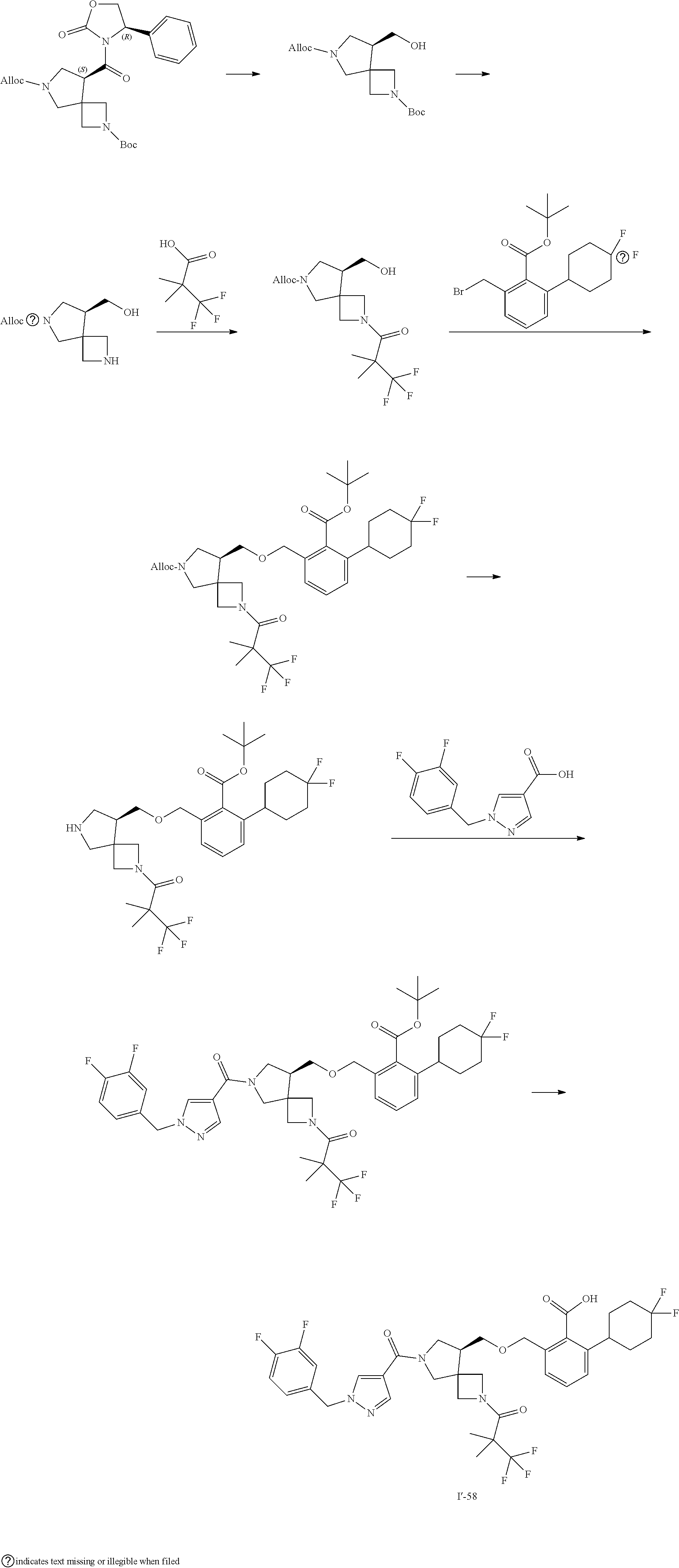

Step 1: allyl (S)-8-(hydroxymethyl)-2,6-diazaspiro[3.4]octane-6-carboxylate To a solution of 6-allyl 2-(tert-butyl) (S)-8-(hydroxymethyl)-2,6-diazaspiro[3.4]octane-2,6-dicarboxylate (1 g, 3.06 mmol, prepared as shown below) in DCM (4 mL) was added TFA (2 mL). The reaction mixture was stirred at room temperature for 2 h. The solvent was removed under vacuum to afford crude allyl (S)-8-(hydroxymethyl)-2,6-diazaspiro[3.4]octane-6-carboxylate (693 mg, 100%) which was used directly in the next step. LCMS m/z=227.2 [M+H]⁺.

Step 2: allyl (S)-8-(hydroxymethyl)-2-(3,3,3-trifluoro-2,2-dimethylpropanoyl)-2,6-diazaspiro[3.4]octane-6-carboxylate To a solution of 3,3,3-trifluoro-2,2-dimethylpropanoic acid (497 mg, 3.06 mmol) in DCM (4 mL) was added HATU (1.16 g, 3.06 mmol) and DIPEA (1.18 g, 9.18 mmol). The reaction mixture was stirred at room temperature for 30 min. Allyl (S)-8-(hydroxymethyl)-2,6-diazaspiro[3.4]octane-6-carboxylate (693 mg, 3.06 mmol) was added and the mixture was stirred at room temperature for another 2 h. The mixture was diluted with water (70 mL) and extracted with DCM (100 mL×2). The combined organic layers were washed with brine, dried over Na₂SO₄, filtered and concentrated. The residue was purified by column chromatography on silica gel (eluent: DCM:MeOH=60:1˜20:1) to afford allyl (S)-8-(hydroxymethyl)-2-(3,3,3-trifluoro-2,2-dimethylpropanoyl)-2,6-diazaspiro[3.4]octane-6-carboxylate (400 mg, 36% yield) as a yellow oil. LCMS m/z=365.1 [M+H]⁺; ¹H NMR (400 MHz, DMSO-d₆) δ 5.98-5.87 (m, 1H), 5.33-5.14 (m, 2H), 4.76 (t, J=4.8 Hz, 1H), 4.53-4.47 (m, 2H), 4.36-4.06 (m, 311), 3.89-3.37 (m, 7H), 2.35-2.26 (m, 1H), 1.34 (s, 6H).

Step 3: allyl (S)-8-(((2-(tert-butoxycarbonyl)-3-(4,4-difluorocyclohexyl)benzyl)oxy)methyl)-2-(3,3,3-trifluoro-2,2-dimethylpropanoyl)-2,6-diazaspiro[3.4]octane-6-carboxylate To a solution of allyl (S)-8-(hydroxymethyl)-2-(3,3,3-trifluoro-2,2-dimethylpropanoyl)-2,6-diazaspiro[3.4]octane-6-carboxylate (400 mg, 1.09 mmol) and tert-butyl 2-(bromomethyl)-6-(4,4-difluorocyclohexyl)benzoate (469 mg, 1.2 mmol) in THF at 0° C. was added sodium hydride (262 mg, 6.54 mmol), Then the mixture was warmed to room temperature and stirred at RT for 3 h. The mixture was diluted with water (30 mL) and extracted with EtOAc (70 mL×2). The combined organic layers were dried over Na₂SO₄, filtered and concentrated. The mixture was purified by prep-TLC (eluent: DCM:MeOH=20:1) to afford allyl (S)-8-(((2-(tert-butoxycarbonyl)-3-(4,4-difluorocyclohexyl)benzyl)oxy)methyl)-2-(3,3,3-trifluoro-2,2-dimethylpropanoyl)-2,6-diazaspiro[3.4]octane-6-carboxylate (520 mg, 71% yield) as a colorless oil. LCMS m/z=673.3 [M+H]⁺; ¹H NMR (400 MHz, DMSO-d₆) δ 7.40-7.30 (m, 2H), 7.27-7.23 (m, 1H), 5.97-5.85 (m, 1H), 5.32-5.14 (m, 2H), 4.52-4.43 (m, 4H), 4.37-3.32 (m, 11H), 2.74-2.64 (m, 1H), 2.21-2.10 (m, 2H), 1.98-1.68 (m, 6H), 1.55 (s, 9H), 1.32-1.25 (m, 6H).

Step 4: tert-butyl (S)-2-(((2-(3,3-difluoro-2,2-dimethylbutanoyl)-2,6-diazaspiro[3.4]octan-8-yl)methoxy)methyl)-6-(4,4-difluorocyclohexyl)benzoate To a solution of allyl (S)-8-(((2-(tert-butoxycarbonyl)-3-(4,4-difluorocyclohexyl)benzyl)oxy)methyl)-2-(3,3,3-trifluoro-2,2-dimethylpropanoyl)-2,6-diazaspiro[3.4]octane-6-carboxylate (520 mg, 0.77 mmol) in DCM (4 mL) was added PPh₃ (50 mg, 0.19 mmol) and pyrrolidine (328 mg, 4.62 mmol) and stirred at room temperature for 10 min. Then Pd(PPh₃)₄ (89 mg, 0.077 mmol) was added the mixture and stirred at RT for 3 h. The mixture was diluted with water (2 mL) and extracted with DCM (10 mL). The organic layer was dried over Na₂SO₄, filtered and concentrated. The mixture was purified by prep-TLC (eluent: DCM:MeOH=20:1) to afford tert-butyl (S)-2-(((2-(3,3-difluoro-2,2-dimethylbutanoyl)-2,6-diazaspiro[3.4]octan-8-yl)methoxy)methyl)-6-(4,4-difluorocyclohexyl)benzoate (240 mg, 53% yield) as a colorless oil. LCMS m/z=589.4 [M+H]⁺; ¹H NMR (400 MHz, DMSO-d₆) δ 7.40-7.23 (m, 3H), 4.51-3.38 (m, 9H), 3.01-2.82 (m, 3H), 2.75-2.65 (m, 1H), 2.27-2.08 (m, 3H), 1.98-1.68 (m, 6H), 1.56 (s, 9H), 1.28 (s, 6H).

Step 5: tert-butyl (S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate To a solution of 1-(3,4-difluorobenzyl)-1H-pyrazole-4-carboxylic acid (31 mg, 0.119 mmol) in DCM (2 mL) was added HATU (45 mg, 0.119 mmol) and DIPEA (46 mg, 0.357 mmol). The reaction mixture was stirred at room temperature for 30 min. tert-butyl (S)-2-(((2-(3,3-difluoro-2,2-dimethylbutanoyl)-2,6-diazaspiro[3.4]octan-8-yl)methoxy)methyl)-6-(4,4-difluorocyclohexyl)benzoate (70 mg, 0.119 mmol) was then added and the mixture was stirred at room temperature for another 2 h. The mixture was diluted with water (15 mL) and extracted with DCM (30 mL×2). The combined organic layers were washed with brine, dried over Na₂SO₄, filtered and concentrated. The residue was purified by column chromatography on silica gel (eluent: DCM:MeOH=30:1) to afford tert-butyl (S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (60 mg, 62% yield) as a colorless oil. LCMS m/z=809.2 [M+H]⁺; ¹H NMR (400 MHz, DMSO-d₆) δ 8.34 (d, J=4.8 Hz, 1H), 7.82 (d, J=12.6 Hz, 1H), 7.45-7.22 (m, 5H), 7.17-7.09 (m, 1H), 5.35 (s, 2H), 4.50-3.38 (m, 14H), 2.19-2.10 (m, 2H), 1.90-1.67 (m, 6H), 1.55-1.49 (m, 9H), 1.29 (s, 6H).

Step 6:(S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-58 To a solution of tert-butyl (S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (60 mg, 0.074 mmol) in DCM (2 mL) was added TFA (1 mL). The reaction mixture was stirred at room temperature for 2 h. The solvent was removed under vacuum and purified by prep-TLC (eluent: DCM:MeOH=20:1) to afford S)-2-(((6-(1-(3,4-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid (50 mg, 90% yield) as a white solid. LCMS m/z=753.4 [M+H]⁺; H NMR (400 MHz, DMSO-d₆) δ 8.36 (s, 1H), 7.83 (d, J=8.2 Hz, 1H), 7.47-7.18 (m, 5H), 7.15-7.09 (m, 1H), 5.35 (s, 2H), 4.55-4.47 (m, 2H), 4.42-3.37 (m, 10H), 2.81-2.71 (m, 1H), 2.65-2.54 (m, 1H), 2.16-2.06 (m, 2H), 1.94-1.78 (m, 4H), 1.75-1.65 (m, 2H), 1.30 (s, 6H).

### Synthesis of 6-allyl 2-(tert-butyl) (S)-8-((R)-2-oxo-4-phenyloxazolidine-3-carbonyl)-2,6-diazaspiro[3.4]octane-2,6-dicarboxylate

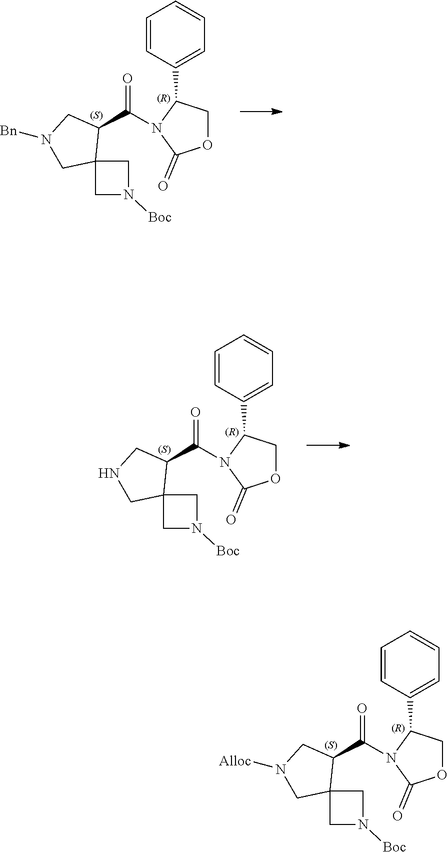

Step 1: tert-butyl (S)-8-((R)-2-oxo-4-phenyloxazolidine-3-carbonyl)-2,6-diazaspiro[3.4]octane-2-carboxylate To a solution of tert-butyl (S)-6-benzyl-8-((R)-2-oxo-4-phenyloxazolidine-3-carbonyl)-2,6-diazaspiro[3.4]octane-2-carboxylate (1.7 g, 3.45 mmol) in EtOAc (13 mL) was added Pd/C (850 mg). The reaction mixture was stirred at 50° C. for 36 h with H₂ balloon. The mixture was filtrated with MeOH and concentrated under vacuum to afford tert-butyl (S)-8-((R)-2-oxo-4-phenyloxazolidine-3-carbonyl)-2,6-diazaspiro[3.4]octane-2-carboxylate (1.1 g, 86%) as a white solid. LCMS m/z=402.2 [M+H]⁺.

Step 2: 6-allyl 2-(tert-butyl) (S)-8-((R)-2-oxo-4-phenyloxazolidine-3-carbonyl)-2,6-diazaspiro[3.4]octane-2,6-dicarboxylate: To a solution of tert-butyl (S)-8-((R)-2-oxo-4-phenyloxazolidine-3-carbonyl)-2,6-diazaspiro[3.4]octane-2-carboxylate (3.5 g, 8.7 mmol) in DCM 40 mL) was added AllocCl (1 g, 8.7 mmol) and TEA (2.6 g, 25.7 mmol). The reaction mixture was stirred at room temperature overnight. The mixture was diluted with water (50 mL) and extracted with DCM (50 mL×3). The combined organic layers were purified by chromatography on silica gel (eluent: PE:EtOAc=1:1) to afford 6-allyl 2-(tert-butyl) (S)-8-((R)-2-oxo-4-phenyloxazolidine-3-carbonyl)-2,6-diazaspiro[3.4]octane-2,6-dicarboxylate as a colorless oil. ¹H NMR (400 MHz, DMSO-d₆) δ 7.42-7.38 (m, 2H), 7.35-7.33 (m, 3H), 5.94-5.79 (m, 1H), 5.47-5.41 (m, 1H), 5.24-5.09 (m, 2H), 4.96-4.89 (m, 1H), 4.78-4.65 (m, 2H), 4.53-4.39 (m, 2H), 4.28-4.13 (m, 2H), 3.99-3.92 (m, 1H), 3.71-3.52 (m, 4H), 3.47-3.37 (m, 1H), 1.37 (s, 9H).

TABLE 18
The compounds listed in Table 18 were synthesized from tert-butyl (S)-2-
(((2-(3,3-difluoro-2,2-dimethylbutanoyl)-2,6-diazaspiro[3.4]octan-8-yl)methoxy)methyl)-6-(4,4-
difluorocyclohexyl)benzoate according to the procedures outlined for I′-58 using the appropriate
commercially available reagents and/or intermediates described elsewhere.

| Cmpd. | | | |
|---|---|---|---|
| No. | Compound | ¹HNMR | LCMS |
| I′-53 | 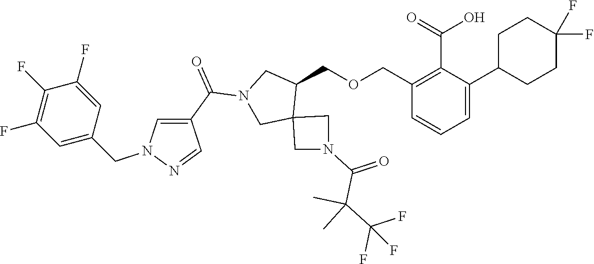 [SMILES: CC(C)(C(=O)N1CC2(CN(C(=O)c3cnn(Cc4cc(F)c(F)c(F)c4)c3)C[C@H]2COCc2cccc(C3CCC(F)(F)CC3)c2C(=O)O)C1)C(F)(F)F] | ¹H NMR (400 MHz, DMSO-d6) δ 8.38 (d, J = 7.6 Hz, 1H), 7.85 (d, J = 8.4 Hz, 1H), 7.31-7.20 (m, 5H), 5.36 (s, 2H), 4.50 (s, 2H), 4.40-3.48 (m, 10H), 2.81-2.71 (m, 1H), 2.09 (s, 2H), 1.94- 1.61 (m, 7H), 1.30 (s, 6H). | m/z = 771.5 [M + H]⁺ |

### 3-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,2,4-oxadiazol-5(4H)-one I′-55

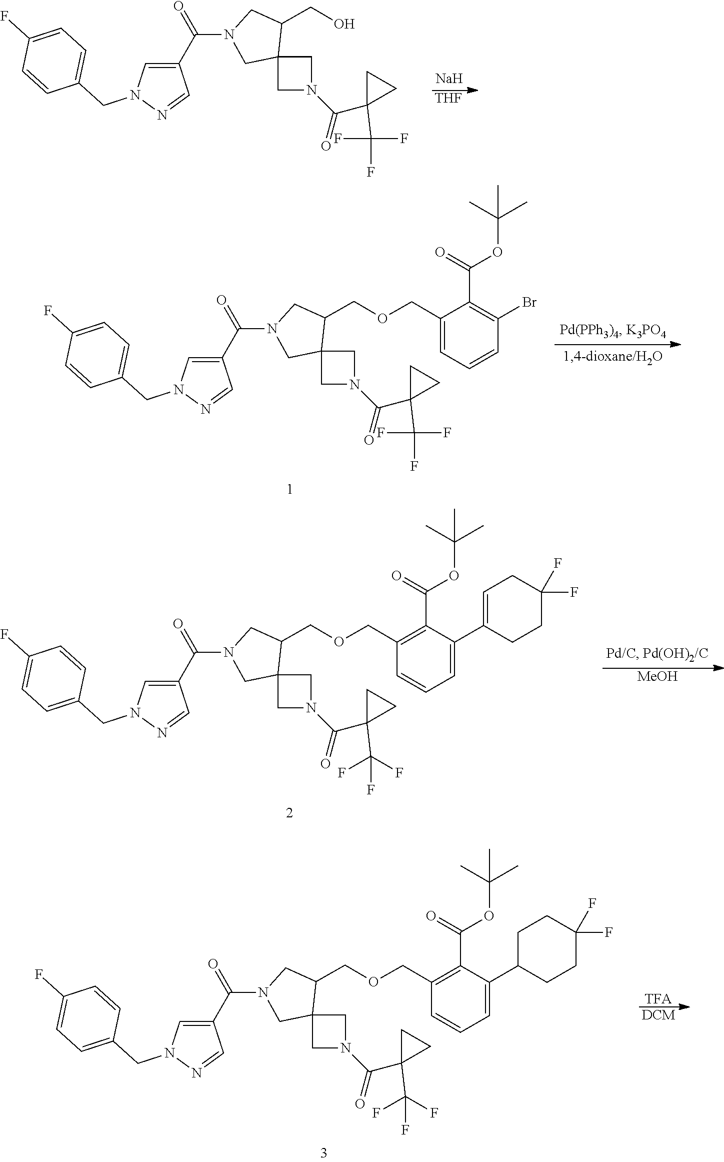

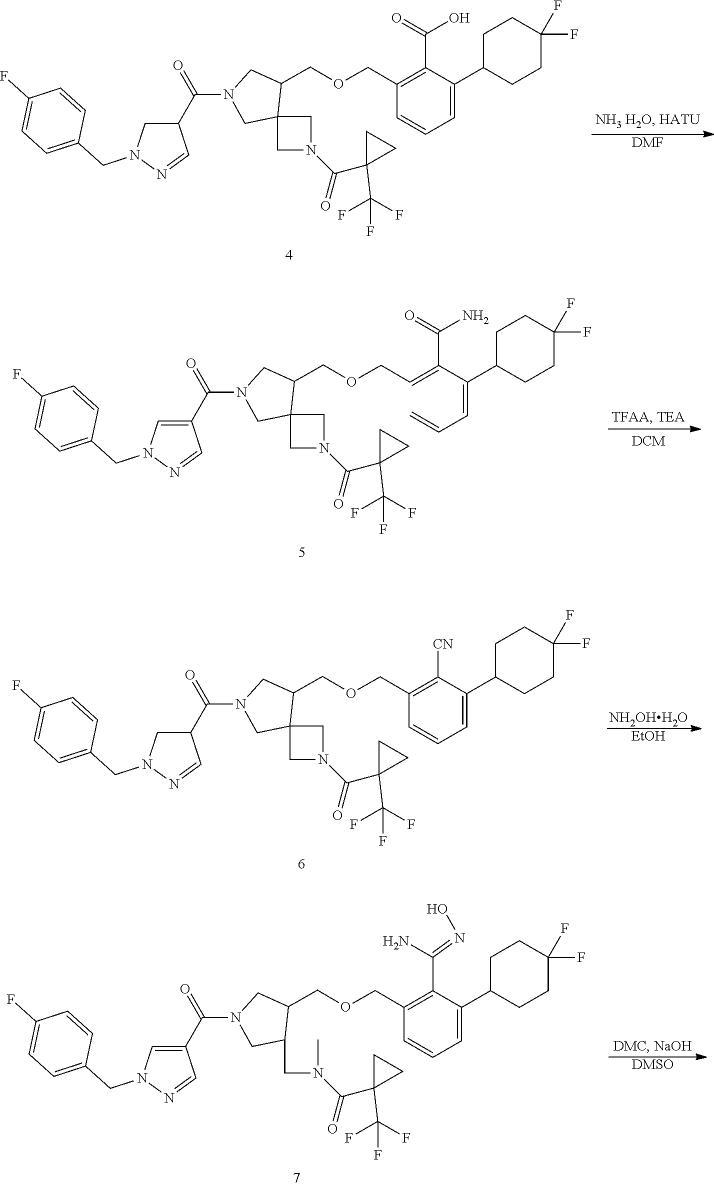

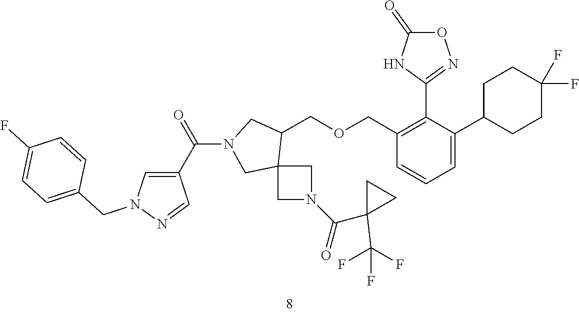 [SMILES: O=C(c1cnn(Cc2ccc(F)cc2)c1)N1CC(COCc2cccc(C3CCC(F)(F)CC3)c2-c2noc(=O)[nH]2)C2(C1)CN(C(=O)C1(C(F)(F)F)CC1)C2]

Step 1: tert-butyl 2-bromo-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate To a solution of (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (0.8 g, 1.67 mmol) in dry THF (8 mL) was added 60% NaH (333 mg, 8.33 mmol) and the mixture stirred at 0° C. for 30 min. tert-butyl 2-bromo-6-(bromomethyl)benzoate (758 mg, 2.16 mmol) was added slowly and the mixture was stirred at room temperature for another 8 hours. The reaction was then diluted with water (70 mL) and extracted with EtOAc (50 mL×3). The combined organic layers were washed with brine, dried over Na₂SO₄, filtered and concentrated. The residue was purified by column chromatography on silica gel (eluent: DCM:MeOH=60:1) to afford tert-butyl 2-bromo-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (980 mg, 78%) as a white solid. LCMS m/z=751.3 [M+H]⁺ ¹H NMR (400 MHz, CD3OD) δ 8.23-8.17 (m, 1H), 7.92-7.88 (m, 1H), 7.58-7.52 (m, 1H), 7.40-7.22 (m, 4H), 7.11-7.03 (m, 2H), 5.37-5.34 (m, 2H), 4.60-4.49 (m, 2H), 4.44-3.44 (m, 10H), 2.71-2.56 (m, 1H), 1.63-1.55 (m, 9H), 1.23-1.09 (m, 4H).

Step 2: tert-butyl 4′,4′-difluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′,3′,4′,5′-tetrahydro-[1,1′-biphenyl]-2-carboxylate To a solution of tert-butyl 2-bromo-6-(((6-(1-(4-fluorobenzyl)-1H-pyrazole-4-carbonyl)-2-(1-(trifluoromethyl) cyclopropane-1-carbonyl)-2,6-diazaspiro[3.4]octan-8-yl)methoxy)methyl)benzoate (200 mg, 0.27 mmol) in 1,4-dioxane/H₂O (2 mL/0.4 mL) was added 2-(4,4-difluorocyclohex-1-en-1-yl)-4,4,5,5-tetramethyl-1,3,2-dioxaborolane (78 mg, 0.28 mmol), K₃PO₄ (172 mg, 0.81 mmol) and Pd(PPh₃)₄ (35 mg, 0.03 mmol). The reaction was stirred at 100° C. under N₂ atmosphere for 3 hours. The mixture was diluted with water (30 mL) and extracted with EtOAc (40 mL×2). The combined organic layers were dried over Na₂SO₄, filtered and concentrated. The residue was purified by prep-TLC (eluent: DCM:MeOH=15:1) to afford tert-butyl 4′,4′-difluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′,3′,4′,5′-tetrahydro-[1,1′-biphenyl]-2-carboxylate (200 mg, quant) as a yellow solid. LCMS m/z=787.4 [M+H]⁺; ¹H NMR (400 MHz, CD3OD) δ 8.23-8.18 (m, 1H), 7.90 (s, 1H), 7.68-7.52 (m, 1H), 7.35-7.26 (m, 4H), 7.20-7.16 (m, 1H), 7.12-7.02 (m, 2H), 5.44 (s, 1H), 5.35 (s, 2H), 4.58 (s, 2H), 4.43-3.43 (m, 10H), 2.71-2.54 (m, 5H), 2.21-2.06 (m, 2H), 1.55-1.49 (m, 9H), 1.20-1.05 (m, 4H).

Step 3: tert-butyl 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate To a solution of tert-butyl 4′,4′-difluoro-3-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-2′,3′,4′,5′-tetrahydro-[1,1′-biphenyl]-2-carboxylate (200 mg, 0.25 mmol) in MeOH (2 mL) was added 10% Pd(OH)₂/C (80 mg) and 10% Pd/C (80 mg) under H₂ atmosphere. The reaction mixture was stirred at 50° C. for 14 h. The mixture was filtered through celite and concentrated to afford tert-butyl 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (150 mg, 75%) as a black solid. LCMS m/z=789.4 [M+H]⁺; ¹H NMR (400 MHz, CD3OD) δ 8.22-8.18 (m, 1H), 7.92-7.88 (m, 1H), 7.35-7.20 (m, 5H), 7.11-7.03 (m, 2H), 5.36 (s, 2H), 4.55 (s, 2H), 4.18-3.44 (m, 9H), 2.81-2.71 (m, 1H), 2.23-1.72 (m, 10H), 1.62-1.57 (m, 9H), 1.19-1.03 (m, 4H).

Step 4: 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid To a solution of tert-butyl 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (500 mg, 0.63 mmol) in DCM (5 mL) was added TFA (2.5 mL). The reaction mixture was stirred at room temperature for 2 h. The solvent was removed under vacuum to afford crude 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (464 mg, 100%) which was used directly in the next step. LCMS m/z=733.4 [M+H]⁺;

Step 5: 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide To a solution of 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoic acid (0.45 g, 0.61 mmol) in DMF (5 mL) was added HATU (234 mg, 0.61 mmol) and DIPEA (238 mg, 1.84 mmol), after stirring for 30 min, NH₃·H₂O (4 mL) was added. The resulting mixture was stirred at room temperature for 3 h. The mixture was diluted with water (100 mL) and extracted with EtOAc (50 mL×3). The combined organic layers were washed with brine, dried over Na₂SO₄, filtered and concentrated to afford 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide (300 mg, 67) as a black solid. LCMS m/z=732.4 [M+H]⁺; ¹H NMR (400 MHz, CD3OD) δ 8.27-8.17 (m, 1H), 7.92-7.88 (m, 1H), 7.36-7.20 (m, 5H), 7.11-7.04 (m, 2H), 5.35 (s, 2H), 4.58 (s, 2H), 4.37-3.58 (m, 10H), 2.75-2.59 (m, 1H), 2.21-1.68 (m, 10H), 1.21-1.14 (m, 4H).

Step 6: 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile To a solution of 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide (140 mg, 0.20 mmol) in DCM (2 mL) was added TEA (31 mg, 0.31 mmol) and TFAA (129 mg, 0.61 mmol). The mixture was stirred at room temperature for 3 h. The solvent was concentrated directly. The residue was purified by prep-TLC (eluent: DCM:MeOH=15:1) to afford 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile (110 mg, 80%) as white solid. LCMS m z=714.3 [M+H]⁺; ¹H NMR (400 MHz, CD3OD) δ 8.21-8.16 (m, 1H), 7.91-7.86 (m, 1H), 7.60-7.54 (m, 1H), 7.45-7.30 (m, 4H), 7.11-7.03 (m, 2H), 5.35 (s, 2H), 4.70-4.65 (m, 2H), 4.50-3.44 (m, 10H), 3.15-3.03 (m, 1H), 2.78-2.60 (m, 1H), 2.25-2.12 (m, 2H), 2.06-1.76 (m, 6H), 1.22-1.11 (m, 4H).

Step 7: (Z)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-N′-hydroxybenzimidamide To a solution of 2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzonitrile (30 mg, 0.04 mmol) in EtOH (0.5 mL) was added NH₂OH·H₂O (50% in water, 1.0 mL). The resulting mixture was heated to 80° C. for 3 h. The reaction was diluted with water and extracted with EtOAc (20 mL×3). The combined organic layers were washed with water, brine, dried over Na₂SO₄ and concentrated. The residue was purified by prep-HPLC to afford (Z)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-N′-hydroxybenzimidamide (7 mg, 22%) as a white solid. LCMS m/z=747.1 [M+H]⁺;

Step 8: 3-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,2,4-oxadiazol-5(4H)-one I′-55 To a solution of (Z)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-N′-hydroxybenzimidamide (30 mg, 0.04 mmol) in DMSO (0.5 mL) was added NaOH (3 mg, 0.06 mmol) and DMC (5 mg, 0.06 mmol). The mixture was stirred at room temperature overnight. The mixture was diluted with water (20 mL) and extracted with EtOAc (50 mL). The organic phase was washed with brine, dried over Na₂SO₄, filtered and concentrated. The mixture was purified by prep-HPLC to afford 3-(2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)phenyl)-1,2,4-oxadiazol-5(4H)-one (13 mg, 41%) as a white solid. LCMS m z=773.4 [M+H]⁺; ¹H NMR (400 MHz, CD3OD) δ 8.22-8.17 (m, 1H), 7.91-7.88 (m, 1H), 7.55-7.43 (m, 2H), 7.40-7.30 (m, 3H), 7.11-7.04 (m, 2H), 5.38-5.34 (m, 2H), 4.56-4.49 (m, 2H), 4.42-4.13 (m, 2H), 4.09-3.43 (m, 8H), 2.72-2.51 (m, 2H), 2.17-2.06 (m, 2H), 1.94-1.80 (m, 6H), 1.25-1.10 (m, 4H).

### (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid I′-51

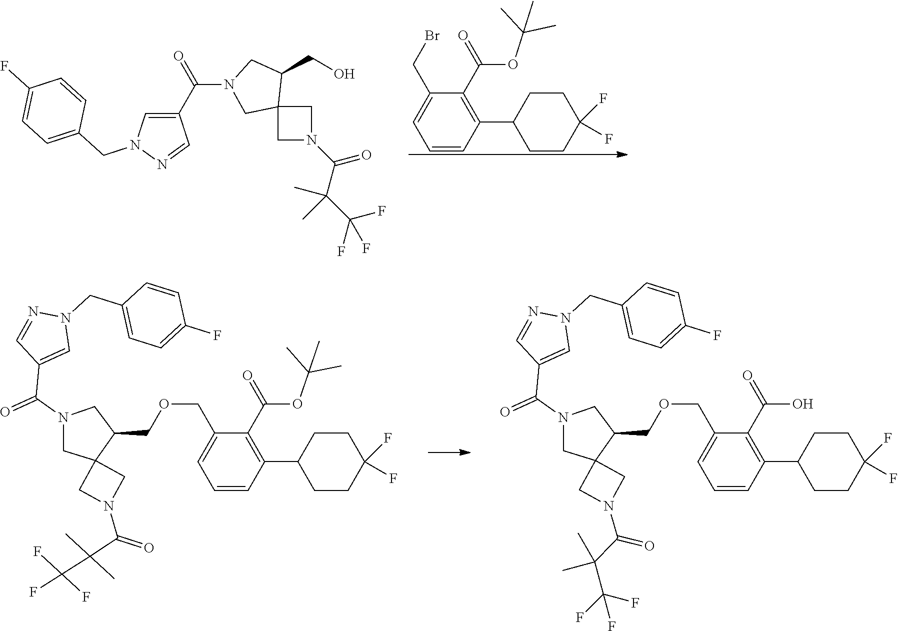 [SMILES: CC(C)(C(=O)N1CC2(CN(C(=O)c3cnn(Cc4ccc(F)cc4)c3)C[C@H]2CO)C1)C(F)(F)F ; CC(C)(C)OC(=O)c1c(CBr)cccc1C1CCC(F)(F)CC1 ; CC(C)(C)OC(=O)c1c(COC[C@@H]2CN(C(=O)c3cnn(Cc4ccc(F)cc4)c3)CC23CN(C(=O)C(C)(C)C(F)(F)F)C3)cccc1C1CCC(F)(F)CC1 ; CC(C)(C(=O)N1CC2(CN(C(=O)c3cnn(Cc4ccc(F)cc4)c3)C[C@H]2COCc2cccc(C3CCC(F)(F)CC3)c2C(=O)O)C1)C(F)(F)F]

Step 1: tert-butyl (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (280 mg, 0.58 mmol) and tert-butyl 2-(bromomethyl)-6-(4,4-difluorocyclohexyl)benzoate (271 mg, 0.7 mmol) in THF (5 mL) at 0° C. was added sodium hydride (116 mg, 2.9 mmol), Then the mixture was warmed to room temperature and stirred at RT for 3 h. The mixture was diluted with water (30 mL) and extracted with EtOAc (70 mL×2). The combined organic layers were dried over Na₂SO₄, filtered and concentrated. The mixture was purified by prep-TLC (eluent: DCM:MeOH=15:1) to afford tert-butyl (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (340 mg, 8700 yield) as a white solid. LCMS m/z=791.4 [M+H]; ¹H NMR (400 MVHz, Chloroform-d) 67.95-7.72 (m, 2H), 7.31 (t, J=7.6 Hz, 1H), 7.25-7.14 (m, 4H), 7.09-7.01 (m, 2H), 5.27 (s, 2H), 4.61-3.36 (m, 14H), 2.80-2.67 (m, 1H), 2.64-2.47 (m, 1H), 2.32-2.10 (m, 3H), 1.96-1.72 (m, 7H), 1.49-1.21 (m, 11H).

Step 2: (S)-2-(4,4-difluorocyclohexyl)-6-(((6-(1-(4-fluorobenzyl)-1H-pyrazole-4-car bonyl)-2-(3,3,3-trifluoro-2,2-dimethylpropanoyl)-2,6-diazaspiro[3.4]octan-8-yl)methoxy)methyl)benzoic acid I′-51 To a solution of tert-butyl (S)-2-(4,4-difluorocyclohexyl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (260 mg, 0.33 mmol) in DCM (3 mL) was added TFA (3 mL). The reaction mixture was stirred at room temperature for 2 h. The solvent was removed under vacuum and purified by prep-TLC (eluent: DCM:MeOH=15:1) to afford (S)-2-(4,4-difluorocyclohexyl)-6-(((6-(I-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (160 mg, 6600 yield) as a white solid. LCMS m/z=735.3 [M+H]˜; ¹H NMR (400 MHz, Methanol-d4) δ 8.24 (d, J=29.3 Hz, 1H), 7.91 (d, J=6.7 Hz, 1H), 7.38-7.27 (m, 4H), 7.26-7.17 (m, 1H), 7.12-7.03 (m, 2H), 5.36 (s, 2H), 4.71-4.22 (m, 4H), 4.08-3.48 (m, 8H), 2.92-2.80 (m, 1H), 2.75-2.56 (m, 1H), 2.22-2.01 (m, 3H), 1.99-1.74 (i, 6H), 1.42-1.33 (n, 6H).

TABLE 19
The compounds listed in Table 19 were synthesized from (S)-3,3,3-trifluoro-
1-(6-(1-(4-fluorobenzyl)-1H-pyrazole-4-carbonyl)-8-(hydroxymethyl)-2,6-diazaspiro[3.4]octan-
2-yl)-2,2-dimethylpropan-1-one according to the procedures outlined for I′-51 using the
appropriate commercially available reagents and/or intermediates described elsewhere.

| Cmpd. | | | |
|---|---|---|---|
| No. | Compound | ¹HNMR | LCMS |
| I′-38 | 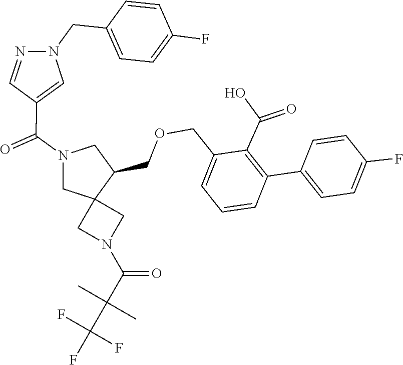 [SMILES: CC(C)(C(=O)N1CC2(CN(C(=O)c3cnn(Cc4ccc(F)cc4)c3)C[C@H]2COCc2cccc(-c3ccc(F)cc3)c2C(=O)O)C1)C(F)(F)F] | ¹H NMR (400 MHz, DMSO-d6) δ 13.14 (s, 1H), 8.34 (s, 1H), 7.81 (d, J = 16.0 Hz, 1H), 7.41 (m, 4H), 7.36 − 7.09 (m, 7H), 5.32 (d, J = 22.2 Hz, 2H), 4.58 (s, 2H), 4.43 − 4.05 (m, 2H), 3.99 − 3.36 (m, 8H), 2.68 − 2.53 (m, 1H), 1.31 (d, J = 6.4 Hz, 6H). | m/z = 711.4 [M + H]⁺ |
| I′-20 | 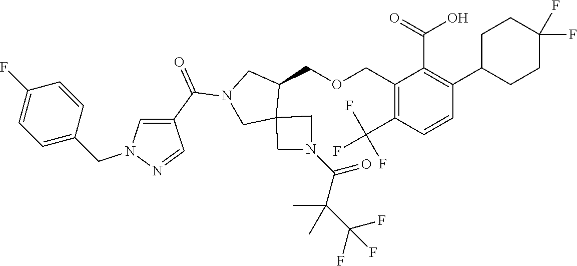 [SMILES: CC(C)(C(=O)N1CC2(CN(C(=O)c3cnn(Cc4ccc(F)cc4)c3)C[C@H]2COCc2c(C(F)(F)F)ccc(C3CCC(F)(F)CC3)c2C(=O)O)C1)C(F)(F)F] | ¹H NMR (400 MHz, Methanol-d4) δ 8.23 (d, J = 41.2 Hz, 1H), 7.89 (d, J = 20.4 Hz, 1H), 7.72 − 7.64 (m, 1H), 7.50 (t, J = 9.3 Hz, 1H), 7.32 (m, 2H), 7.11 − 7.05 (m, 2H), 5.35 (d, J = 5.1 Hz, 2H), 4.75 (t, J = 9.2 Hz, 2H), 4.65 − 3.61 (m, 9H), 2.99 − 2.88 (m, 1H), 2.72 − 2.54 (m, 1H), 2.18 − 1.78 (m, 9H), 1.33 (d, J = | m/z = 803.4 [M + H]⁺ |
| | | 28.3 Hz, 6H). | |
| I′-21 | 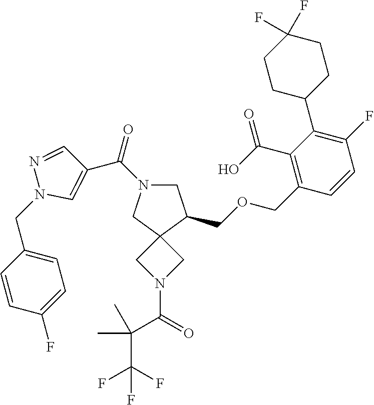 [SMILES: CC(C)(C(=O)N1CC2(CN(C(=O)c3cnn(Cc4ccc(F)cc4)c3)C[C@H]2COCc2ccc(F)c(C3CCC(F)(F)CC3)c2C(=O)O)C1)C(F)(F)F] | ¹H NMR (400 MHz, Methanol-d4) δ 8.23 (d, J = 21.6 Hz, 1H), 7.91 (d, J = 6.8 Hz, 1H), 7.35 − 7.23 (m, 3H), 7.11 − 7.01 (m, 3H), 5.35 (s, 2H), 4.54 (s, 2H), 4.46 − 3.47 (m, 10H), 2.85 − 2.76 (m, 1H), 2.68 − 2.57 (m, 1H), 2.26 − 2.07 (m, 4H), 1.86 − 1.71 (m, 4H), 1.38 (s, 6H). | m/z = 753.3 [M + H]⁺ |
| I′-13 | 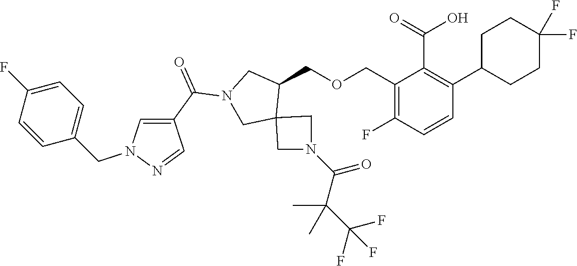 [SMILES: CC(C)(C(=O)N1CC2(CN(C(=O)c3cnn(Cc4ccc(F)cc4)c3)C[C@H]2COCc2c(F)ccc(C3CCC(F)(F)CC3)c2C(=O)O)C1)C(F)(F)F] | ¹H NMR (400 MHz, Methanol-d4) δ 8.29 − 8.17 (m, 1H), 7.90 (d, J = 10.8 Hz, 1H), 7.37 − 7.28 (m, 3H), 7.12 − 7.04 (m, 3H), 5.37 − 5.33 (m, 2H), 4.71 − 4.60 (m, 2H), 4.56 − 4.17 (m, 2H), 4.04 − 3.46 (m, 8H), 2.88 − 2.78 (m, 1H), 2.69 − 2.53 (m, 1H), 2.17 − 2.05 (m, 2H), 1.96 − 1.84 (m, 3H), 1.83 − 1.71 (m, 3H), 1.41 − 1.33 (m, 6H). | m/z = 753.4 [M + H]⁺ |
| I′-94 | 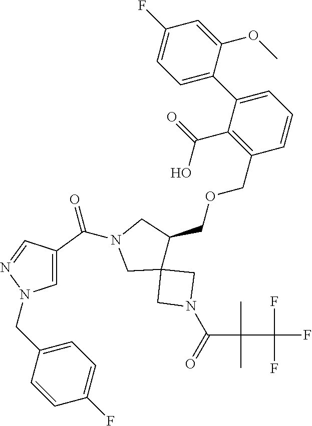 [SMILES: COc1cc(F)ccc1-c1cccc(COC[C@@H]2CN(C(=O)c3cnn(Cc4ccc(F)cc4)c3)CC23CN(C(=O)C(C)(C)C(F)(F)F)C3)c1C(=O)O] | ¹H NMR (400 MHz, DMSO-d6) δ 8.36 (s, 1H), 7.81 (d, J = 23.2 Hz, 1H), 7.41 − 7.30 (m, 3H), 7.22 − 7.11 (m, 3H), 7.00 (s, 2H), 6.91 − 6.81 (m, 1H), 6.73 − 6.55 (m, 1H), 5.32 (d, J = 22.1 Hz, 2H), 4.60 − 4.51 (m, 2H), 4.39 − 4.20 (m, 1H), 4.17 − 3.90 (m, 2H), 3.88 − 3.48 (m, 10H), 2.62 (s, 1H), 1.33 (s, 6H). | m/z = 741.4 [M + H]⁺ |
| I′-95 | 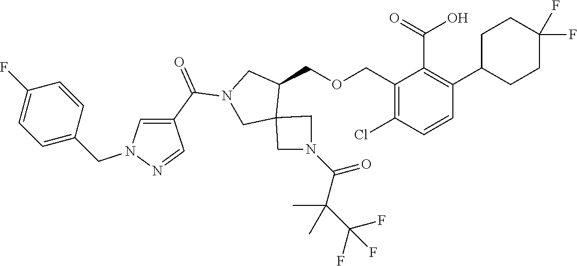 [SMILES: CC(C)(C(=O)N1CC2(CN(C(=O)c3cnn(Cc4ccc(F)cc4)c3)C[C@H]2COCc2c(Cl)ccc(C3CCC(F)(F)CC3)c2C(=O)O)C1)C(F)(F)F] | ¹H NMR (DMSO, 400 MHz) δ 13.54 (s, 1H), 8.32 (s, 1H), 7.80 (d, J = 10.0 Hz, 1H), 7.47 − 7.31 (m, 4H), 7.18 (d, J = 8.6 Hz, 2H), 5.34 (s, 2H), 4.60 − 4.52 (m, 2H), 4.32 (s, 1H), 4.23 − 4.02 (m, 1H), 3.92 − 3.44 (m, 8H), 2.70 (s, 1H), 2.09 (s, 2H), 1.96 − 1.62 (m, 7H), 1.30 (s, 6H) | m/z = 769.4 [M + H]⁺ |

### (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoic acid I′-52

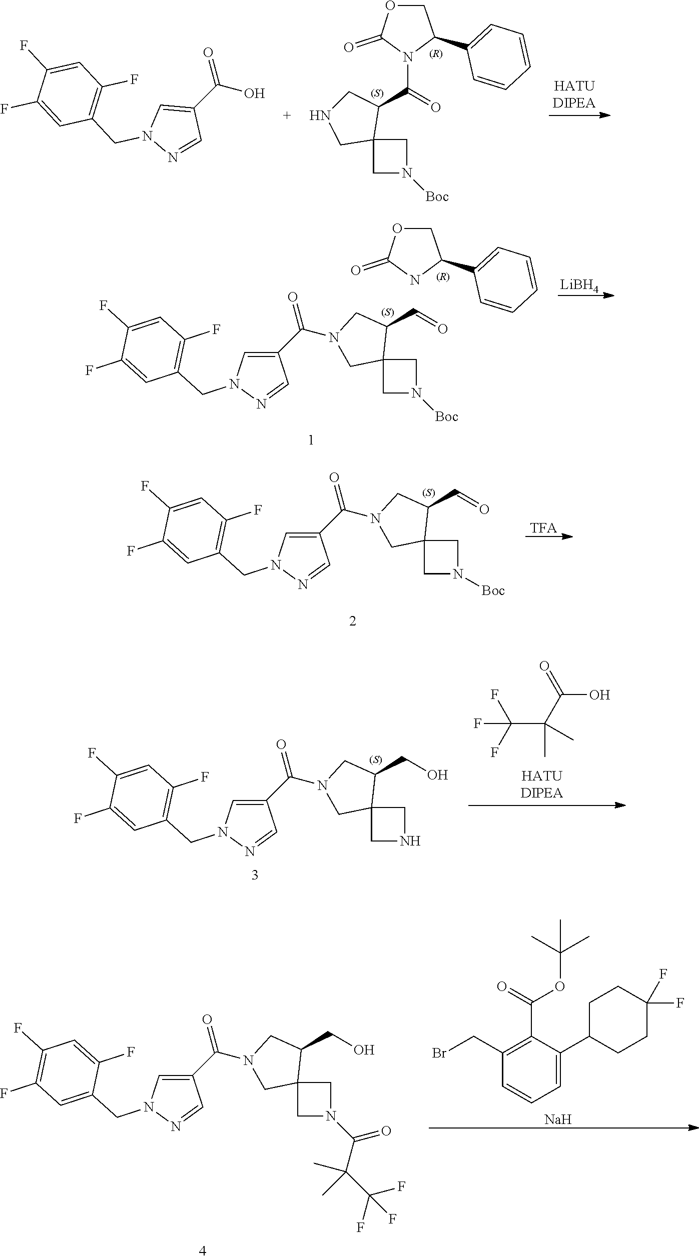

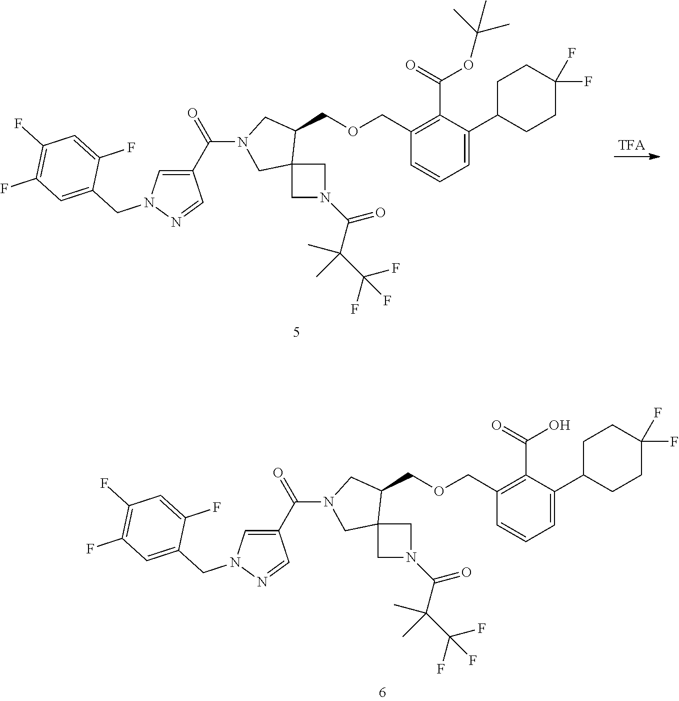 [SMILES: CC(C)(C)OC(=O)c1c(COC[C@@H]2CN(C(=O)c3cnn(Cc4cc(F)c(F)cc4F)c3)CC23CN(C(=O)C(C)(C)C(F)(F)F)C3)cccc1C1CCC(F)(F)CC1 ; CC(C)(C(=O)N1CC2(CN(C(=O)c3cnn(Cc4cc(F)c(F)cc4F)c3)C[C@H]2COCc2cccc(C3CCC(F)(F)CC3)c2C(=O)O)C1)C(F)(F)F]

Step 1: tert-butyl (S)-8-((R)-2-oxo-4-phenyloxazolidine-3-carbonyl)-6-(1-(2,4,5-trifluorobenzyl)-1H-pyrazole-4-carbonyl)-2,6-diazaspiro[3.4]octane-2-carboxylate To a solution of 1-(2,4,5-trifluorobenzyl)-1H-pyrazole-4-carboxylic acid (255 mg, 0.996 mmol) in DMF (5 mL) was added HATU (378.7 mg, 0.996 mmol) and DIPEA (0.5 mL, 2.99 mmol). After stirring for 15 min, tert-butyl (S)-8-((R)-2-oxo-4-phenyloxazolidine-3-carbonyl)-2,6-diazaspiro[3.4]octane-2-carboxylate (400 mg, 0.996 mmol) was added and the reaction stirred at room temperature for another 3 h. The mixture was diluted with water (20 mL) and extracted with EtOAc (15 mL×3). The combined organic layers were washed with water, brine, dried over Na₂SO₄, filtered and concentrated. The crude was purified by prep-TLC (eluent: DCM:MeOH=15:1) to afford tert-butyl (S)-8-((R)-2-oxo-4-phenyloxazolidine-3-carbonyl)-6-(1-(2,4,5-trifluorobenzyl)-1H-pyrazole-4-carbonyl)-2,6-diazaspiro[3.4]octane-2-carboxylate (286 mg, 44%) as a white solid. LCMS m/z=640.3 [M+H]⁺; ¹HNMR (400 MHz, CD₃OD) δ 8.16 (s, 1H), 7.83 (d, J=10.5 Hz, 1H), 7.39-7.08 (m, 7H), 5.50-5.40 (m, 2H), 5.37 (s, 1H), 4.82-4.74 (m, 1H), 4.52-3.75 (m, 10H), 1.44 (d, J=5.9 Hz, 9H).

Step 2: tert-butyl (S)-8-(hydroxymethyl)-6-(1-(2,4,5-trifluorobenzyl)-1H-pyrazole-4-carbonyl)-2,6-diazaspiro[3.4]octane-2-carboxylate To a solution of tert-butyl (S)-8-((R)-2-oxo-4-phenyloxazolidine-3-carbonyl)-6-(1-(2,4,5-trifluorobenzyl)-1H-pyrazole-4-carbonyl)-2,6-diazaspiro[3.4]octane-2-carboxylate (480 mg, 0.75 mmol) in THE (10 mL) was added LiBH₄ (2 M in THF, 0.26 mL, 0.53 mmol) at 0° C. The mixture was stirred at room temperature for 1 h. The mixture was diluted with water (20 mL), extracted with EtOAc (20 mL×3) and the combined organic layers were washed with brine, dried over Na₂SO₄, filtered and concentrated. The crude was purified by prep-TLC (eluent: DCM:MeOH=15:1) to afford tert-butyl (S)-8-(hydroxymethyl)-6-(1-(2,4,5-trifluorobenzyl)-1H-pyrazole-4-carbonyl)-2,6-diazaspiro[3.4]octane-2-carboxylate (286 mg, 72%) as a white solid. LCMS m/z=481.1 [M+H]⁺; ¹HNMR (400 MHz, CD₃OD) δ 8.26-8.21 (m, 1H), 7.92 (d, J=4.9 Hz, 1H), 7.33-7.21 (m, 2H), 5.41 (s, 2H), 4.25-3.50 (m, 11H), 2.54-2.40 (m, 1H), 1.44 (d, J=8.1 Hz, 9H).

Step 3: (S)-(8-(hydroxymethyl)-2,6-diazaspiro[3.4]octan-6-yl)(1-(2,4,5-trifluorobenzyl)-1H-pyrazol-4-yl)methanone To a solution of tert-butyl (S)-8-(hydroxymethyl)-6-(1-(2,4,5-trifluorobenzyl)-1H-pyrazole-4-carbonyl)-2,6-diazaspiro[3.4]octane-2-carboxylate (280 mg, 0.58 mmol) in DCM (8 mL) was added TFA (2 mL). The resulting mixture was stirred at room temperature for 1 hours. The solvent was removed under vacuum to afford (S)-(8-(hydroxymethyl)-2,6-diazaspiro[3.4]octan-6-yl)(1-(2,4,5-trifluorobenzyl)-1H-pyrazol-4-yl)methanone (480 mg, quant) as a yellow oil. LCMS m/z=381.2 [M+H]⁺.

Step 4: (S)-3,3,3-trifluoro-1-(8-(hydroxymethyl)-6-(1-(2,4,5-trifluorobenzyl)-1H-pyrazole-4-carbonyl)-2,6-diazaspiro[3.4]octan-2-yl)-2,2-dimethylpropan-1-one To a solution of 3,3,3-trifluoro-2,2-dimethylpropanoic acid (86 mg, 0.55 mmol) in DMF (5 mL) was added HATU (190 mg, 0.5 mmol) and DIPEA (0.5 mL, 2.99 mmol). After stirring for 15 min. (S)-(8-(hydroxymethyl)-2,6-diazaspiro[3.4]octan-6-yl)(1-(2,4,5-trifluorobenzyl)-1H-pyrazol-4-yl)methanone (189 mg, 0.5 mmol) was added and the reaction stirred at room temperature for another 2 h. The mixture was diluted with water (20 mL) and extracted with EtOAc (25 mL×2). The combined organic layers were washed with brine, dried over Na₂SO₄, filtered and concentrated. The mixture was purified by prep-TLC (eluent: DCM:MeOH=10:1) to afford (S)-3,3,3-trifluoro-1-(8-(hydroxymethyl)-6-(1-(2,4,5-trifluorobenzyl)-1H-pyrazole-4-carbonyl)-2,6-diazaspiro[3.4]octan-2-yl)-2,2-dimethylpropan-1-one (195 mg, 75%) as a yellow solid. LCMS m/z=519.3 [M+H]⁺; ¹HNMR (400 MHz, CD3OD) δ 8.26-8.21 (m, 1H), 7.95-7.90 (m, 1H), 7.28-7.19 (m, 2H), 5.41 (s, 2H), 4.81-4.25 (m, 3H), 4.11-3.46 (m, 8H), 2.64-2.37 (m, 1H), 1.45-1.40 (m, 6H).

Step 5: tert-butyl (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoate To a solution of(S)-3,3,3-trifluoro-1-(8-(hydroxymethyl)-6-(1-(2,4,5-trifluorobenzyl)-1H-pyrazole-4-carbonyl)-2,6-diazaspiro[3.4]octan-2-yl)-2,2-dimethylpropan-1-one (120 mg, 0.23 mmol) and tert-butyl 2-(bromomethyl)-6-(4,4-difluorocyclohexyl)benzoate (113 mg, 0.28 mmol) in anhydrous THF (2.0 mL) at 0° C. was added NaH (46 mg, 1.15 mmol). The mixture was stirred at room temperature overnight. The mixture was diluted with water (10 mL) and extracted with EtOAc (15 mL×3). The combined organic phase was washed with brine, dried over Na₂SO₄, filtered and concentrated. The mixture was purified by prep-TLC (eluent: DCM:MeOH=10:1) to afford tert-butyl (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoate (105 mg, 44%) as a white solid. LCMS m/z=827.3[M+H]⁺; ¹ HNMR (400 MHz, CD3OD) δ 8.26-8.18 (m, 1H), 7.94-7.89 (m, 1H), 7.37-7.17 (m, 5H), 5.40 (s, 2H), 4.60-3.47 (m, 12H), 2.83-2.54 (m, 2H), 2.16 (s, 2H), 1.96-1.74 (m, 6H), 1.63-1.56 (m, 9H), 1.42-1.32 (m, 6H).

Step 6: (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoic acid To a solution of tert-butyl (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoate (100 mg, 0.12 mmol) in DCM (2 mL) was added TFA (1 mL). The resulting mixture was stirred at room temperature for 2 h. The solvent was removed and the residue was purified by prep-TLC (eluent: DCM:MeOH=15:1) to afford (S)-2-(4,4-difluorocyclohexyl)-6-(((2-(3,3,3-trifluoro-2,2-dimethylpropanoyl)-6-(1-(2,4,5-trifluorobenzyl)-1H-pyrazole-4-carbonyl)-2,6-diazaspiro[3.4]octan-8-yl)methoxy)methyl)benzoic acid (80 mg, 86%) as a white solid. LCMS m z=771.4[M+H]⁺; ¹HNMR (400 MHz, CD₃OD) δ 8.24 (d, J=23.4 Hz, 1H), 7.92 (d, J=11.8 Hz, 1H), 7.36-7.19 (m, 5H), 5.40 (s, 2H), 4.63-3.47 (m, 12H), 2.90-2.79 (m, 1H), 2.71-2.57 (m, 1H), 2.19-2.08 (m, 211), 1.96-1.73 (m, 6H), 1.43-1.32 (m, 6H).

(S)-2-(((6-(1-(3,5-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-45

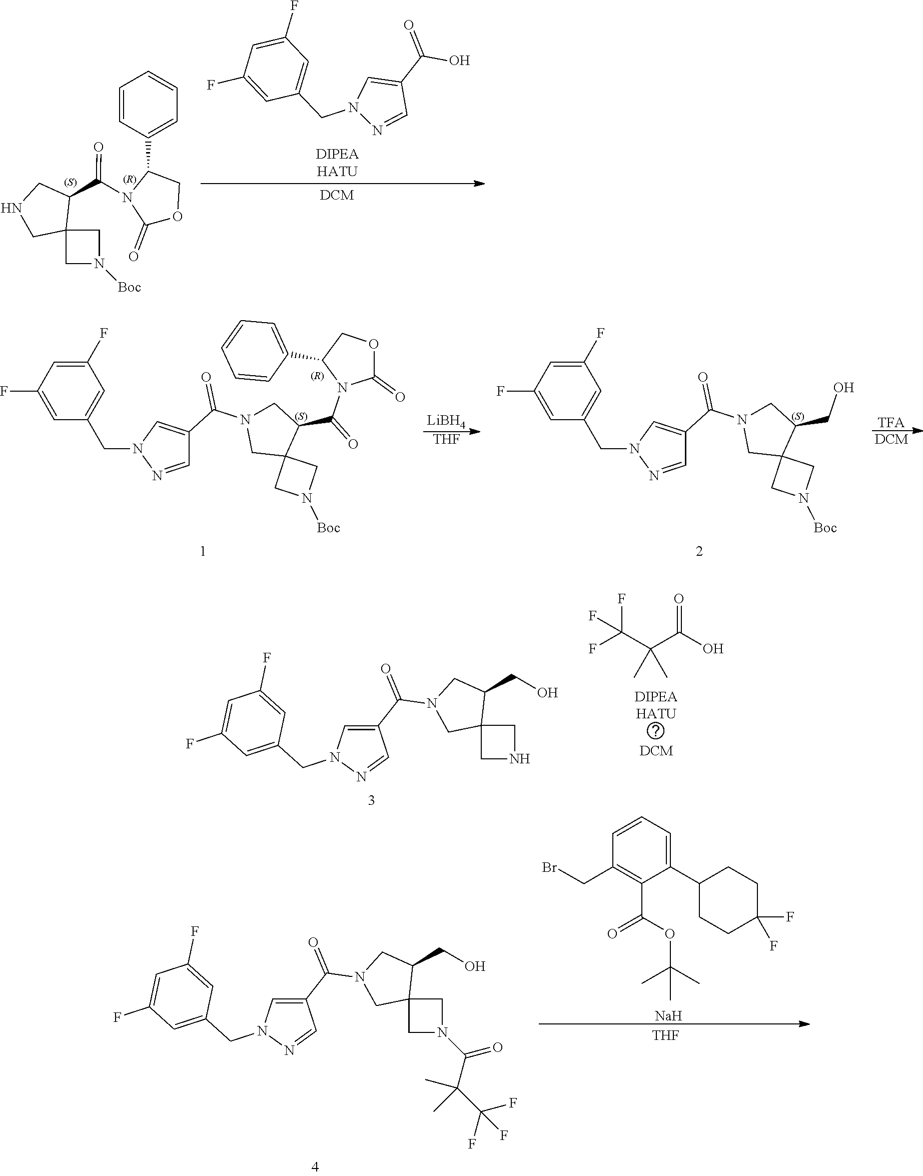

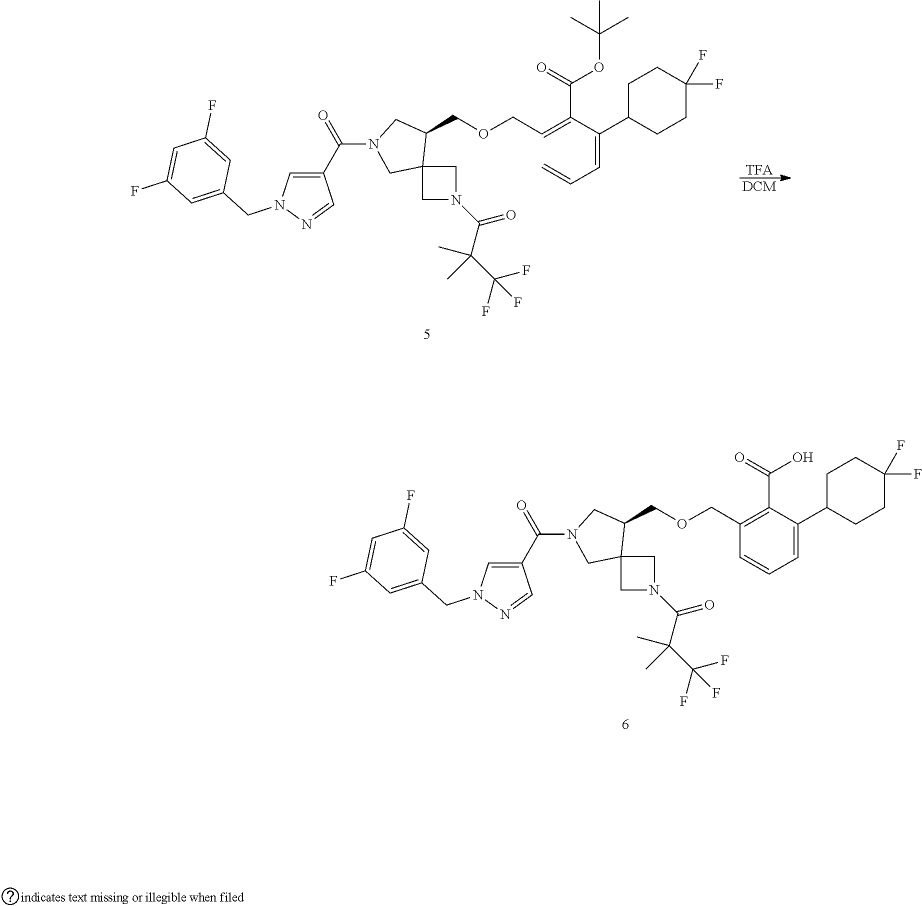

Step 1: tert-butyl (S)-6-(1-(3,5-difluorobenzyl)-1H-pyrazole-4-carbonyl)-8-((R)-2-oxo-4-phenyloxazolidine-3-carbonyl)-2,6-diazaspiro[3.4]octane-2-carboxylate To a solution of 1-(3,5-difluorobenzyl)-1H-pyrazole-4-carboxylic acid (720 mg, 3.02 mmol) in DCM (10 mL) was added HATU (1.15 g, 3.02 mmol) and DIPEA (1.95 g, 15.11 mmol). After stirring for 15 min. tert-butyl (S)-8-((R)-2-oxo-4-phenyloxazolidine-3-carbonyl)-2,6-diazaspiro[3.4]octane-2-carboxylate (1.45 g, 3.63 mmol) was added and the mixture was stirred at room temperature for 2 h. The mixture was diluted with water (100 mL) and extracted with DCM (50 mL×2). The combined organic layers were washed with brine, dried over Na₂SO₄, filtered and concentrated. The residue obtained was purified by column chromatography on silica gel (eluent: Pet.ether:EtOAc=2:1 to 1:2) to afford tert-butyl (S)-6-(1-(3,5-difluorobenzyl)-1H-pyrazole-4-carbonyl)-8-((R)-2-oxo-4-phenyloxazolidine-3-carbonyl)-2,6-diazaspiro[3.4]octane-2-carboxylate (1.3 g, 70%) as a white solid. LCMS: m/z=622.2 [M+H]⁺, ¹HNMR (400 MHz, DMSO-d₆) δ 8.39-8.31 (m, 1H), 7.85-7.77 (m, 1H), 7.37-7.27 (m, 1H), 7.26-7.16 (m, 3H), 7.12-7.06 (m, 1H), 6.99-6.92 (m, 2H), 5.47-5.33 (m, 3H), 4.80-4.69 (m, 1H), 4.40-3.96 (m, 5H), 3.90-3.76 (m, 5H), 1.99 (s, 1H), 1.41-1.35 (m, 9H).

Step 2: tert-butyl (S)-6-(1-(3,5-difluorobenzyl)-1H-pyrazole-4-carbonyl)-8-(hydroxymethyl)-2,6-diazaspiro[3.4]octane-2-carboxylate To a solution of tert-butyl (S)-6-(1-(3,5-difluorobenzyl)-1H-pyrazole-4-carbonyl)-8-((R)-2-oxo-4-phenyloxazolidine-3-carbonyl)-2,6-diazaspiro[3.4]octane-2-carboxylate (500 mg, 0.75 mmol) in dry THF (5 mL) was added LiBH₄ (2 M in THF, 0.3 mL, 0.6 mmol) at −78° C., The mixture was stirred at room temperature under N₂ atomosphere for 3 h. The mixture was diluted with water (100 mL) and extracted with EtOAc (50 mL×3). The organic layers were washed with brine, dried over Na₂SO₄, filtered and concentrated. The crude was purified by RP-column (65% water in MeCN) to afford tert-butyl (S)-6-(1-(3,5-difluorobenzyl)-1H-pyrazole-4-carbonyl)-8-(hydroxymethyl)-2,6-diazaspiro[3.4]octane-2-carboxylate (250 mg, 67%) as a white solid. LCMS: m/z=463.2 [M+H]⁺; ¹HNMR (400 MHz, DMSO-d₆) δ 8.40-8.34 (m, 1H), 7.90-7.78 (m, 1H), 7.24-7.13 (m, 1H), 7.02-6.92 (m, 2H), 5.39 (s, 2H), 4.79-4.70 (m, 1H), 4.17-3.35 (m, 10H), 2.42-2.22 (m, 1H), 1.37 (d, J=4.6 Hz, 9H).

Step 3: (S)-(1-(3,5-difluorobenzyl)-1H-pyrazol-4-yl)(8-(hydroxymethyl)-2,6-diazaspiro[3.4]octan-6-yl)methanone To a solution of tert-butyl (S)-6-(1-(3,5-difluorobenzyl)-1H-pyrazole-4-carbonyl)-8-(hydroxymethyl)-2,6-diazaspiro[3.4]octane-2-carboxylate (200 mg, 0.4 mmol) in DCM (2 mL) was added TFA (0.6 mL). The reaction mixture was stirred at room temperature for 1 h. The solvent was removed under vacuum to afford (S)-(1-(3,5-difluorobenzyl)-1H-pyrazol-4-yl)(8-(hydroxymethyl)-2,6-diazaspiro[3.4]octan-6-yl)methanone (156 mg, quant.) which was used directly in the next step. LCMS: m/z=363.1 [M+H]⁺.

Step 4: (S)-1-(6-(1-(3,5-difluorobenzyl)-1H-pyrazole-4-carbonyl)-8-(hydroxymethyl)-2,6-diazaspiro[3.4]octan-2-yl)-3,3,3-trifluoro-2,2-dimethylpropan-1-one: To a solution of 3,3,3-trifluoro-2,2-dimethylpropanoic acid (112 mg, 0.717 mmol) in DCM (2 mL) was added HATU (136 mg, 0.358 mmol) and DIPEA (463 mg, 3.59 mmol), after stirring for 15 min. (S)-(1-(3,5-difluorobenzyl)-1H-pyrazol-4-yl)(8-(hydroxymethyl)-2,6-diazaspiro[3.4]octan-6-yl)methanone (156 mg, 0.358 mmol) was added and the mixture was stirred at room temperature for 2 h. The mixture was diluted with water (50 mL) and extracted with DCM (30 mL×2). The combined organic layers were washed with brine, dried over Na₂SO₄, filtered and concentrated. The residue obtained was purified by prep-TLC (DCM:MeOH=20:1) to afford (S)-1-(6-(1-(3,5-difluorobenzyl)-1H-pyrazole-4-carbonyl)-8-(hydroxymethyl)-2,6-diazaspiro[3.4]octan-2-yl)-3,3,3-trifluoro-2,2-dimethylpropan-1-one (120 mg, 49%) as a white solid. LCMS: m/z=501.1[M+H]⁻; ¹HNMR (400 MHz, Chloroform-d) δ 7.97-7.77 (m, 2H), 6.84-6.68 (m, 3H), 5.29 (s, 2H), 4.40-3.51 (m, 10H), 2.59-2.39 (m, 1H), 1.41 (s, 6H).

Step 5: tert-butyl (S)-2-(((6-(1-(3,5-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate To a solution of (S)-1-(6-(1-(3,5-difluorobenzyl)-1H-pyrazole-4-carbonyl)-8-(hydroxymethyl)-2,6-diazaspiro[3.4]octan-2-yl)-3,3,3-trifluoro-2,2-dimethylpropan-1-one (100 mg, 0.3 mmol) and di-tert-butyl 2-(bromomethyl)-6-(4,4-difluorocyclohexyl)terephthalate (93 mg, 0.24 mmol) in dry THF (2 mL) was added NaH (40 mg, 1.0 mmol) at 0° C., the mixture was stirred at room temperature under N₂ atmosphere for 3 h. The mixture was diluted with water (50 ml) and extracted with EtOAc (30 mL×3). The organic layers were washed with brine, dired over Na₂SO₄, filtered and concentrated. The crude was purified by Prep-TLC (DCM:MeOH=20:1) to afford tert-butyl (S)-2-(((6-(1-(3,5-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (80 mg, 50%) as a colorless oil. LCMS: m/z=808.3[M+H]⁺;

Step 6: (S)-2-(((6-(1-(3,5-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid To a solution of tert-butyl (S)-2-(((6-(1-(3,5-difiuorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (80 mg, 0.098 mmol) in DCM (2 mL) was added TFA (1 mL). The reaction mixture was stirred at room temperature for 1 h. The solvent was removed under vacuum and the residue purified by prep-HPLC to (S)-2-(((6-(1-(3,5-difluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid (27.6 mg, 37.7%) as white solid. LCMS: m/z=753.4[M+H]⁺; ¹HNMR (400 MHz, Chloroform-d) δ 8.01-7.90 (m, 1H), 7.84 (s, 1H), 7.37-7.28 (m, 2H), 7.16-7.08 (m, 1H), 6.82-6.70 (m, 3H), 5.30 (s, 2H), 4.92-4.63 (m, 1H), 4.58-4.16 (m, 4H), 4.08-3.78 (m, 4H), 3.70-3.61 (m, 2H), 3.54-3.42 (m, 1H), 2.96-2.88 (m, 1H), 2.58-2.48 (m, 1H), 2.24-2.13 (m, 2H), 2.06-1.97 (m, 1H), 1.94-1.74 (m, 5H), 1.47-1.40 (m, 6H).

### ((S)-8-(((3-(4,4-difluorocyclohexyl)-2-(2H-tetrazol-5-yl)benzyl)oxy)methyl)-2-((S)-2,2-dimethylcyclopropane-1-carbonyl)-2,6-diazaspiro[3.4]octan-6-yl)(1-(4-fluorobenzyl)-1H-pyrazol-4-yl)methanone I′-43

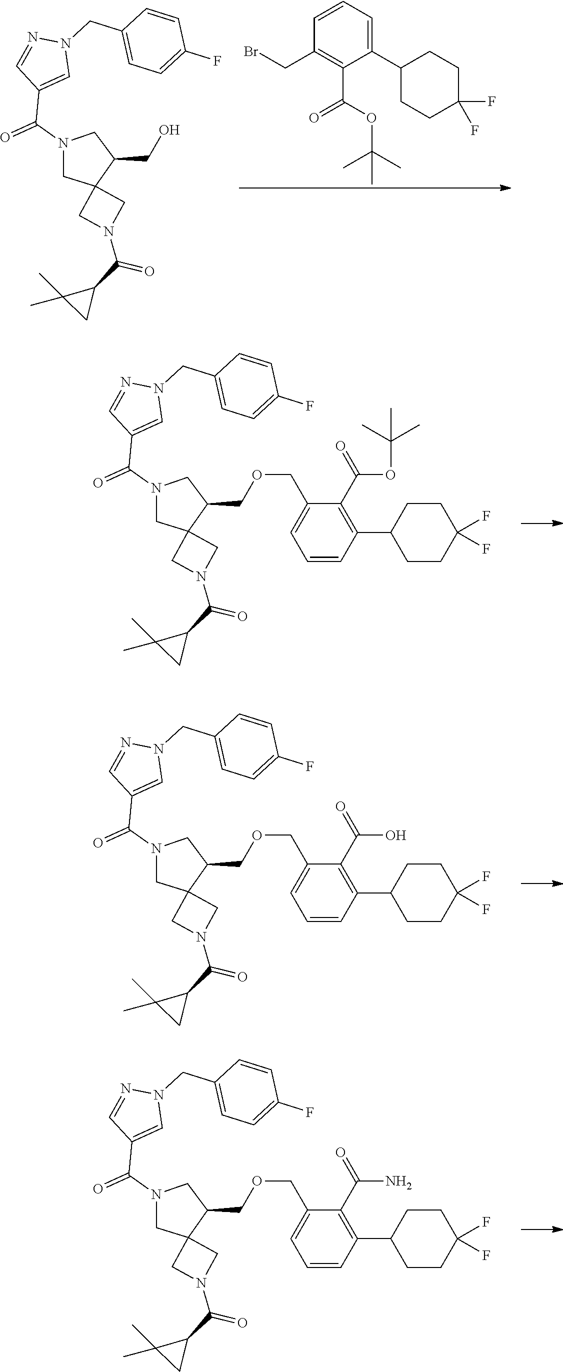

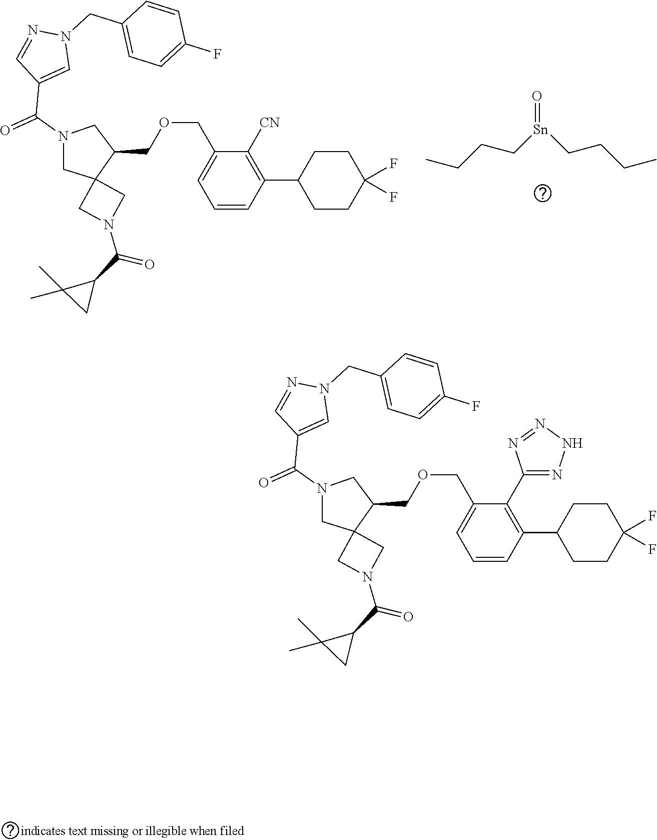

Step 1: tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate: To a solution of ((S)-2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (400 mg, 0.91 mmol), and tert-butyl 2-(bromomethyl)-6-(4,4-difluorocyclohexyl)benzoate (424.2 mg, 1.09 mmol) in THF (10 mL) at 0° C. was added sodium hydride (91 mg, 2.27 mmol), Then the mixture was warmed to room temperature and stirred at room temperature for 2 h. The mixture was diluted with water (20 mL) and extracted with EtOAc (20 mL×3). The combined organic layers were dried over Na₂SO₄, filtered and concentrated. The mixture was purified by prep-TLC (DCM/MeOH-15/1) to afford tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (571 mg, 83.9% yield) as a white solid. LCMS m/z=749.35 [M+H]⁺; ¹H NMR (400 MHz, DMSO-d6) δ 8.33 (d, J=8.0 Hz, 1H), 7.81 (d, J=12.8 Hz, 1H), 7.35-7.13 (m, 7H), 5.34 (s, 2H), 4.52-4.44 (m, 2H), 4.26-4.17 (m, 1H), 4.06-3.34 (m, 10H), 2.73-2.53 (m, 2H), 2.21-2.07 (m, 2H), 1.93-1.66 (m, 6H), 1.56-1.49 (m, 9H), 1.11-1.01 (m, 5H), 0.99 (s, 1H), 0.88-0.82 (m, 1H), 0.69-0.61 (m, 1H).

Step 2: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid: To a solution of tert-butyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate (570 mg, 0.76 mmol) in DCM (6 mL) was added TFA (3.5 mL). The reaction mixture was stirred at room temperature for 2 h. The mixture was purified by prep-TLC (DCM/MeOH=10/1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (480 mg, 91%) as a white solid. LCMS m/z=693.3 [M+H]⁺; ¹H NMR (400 MHz, DMSO-d₆) δ 8.39-8.32 (m, 1H), 7.82 (d, J=8.6 Hz, 1H), 7.37-7.10 (m, 7H), 5.34 (s, 2H), 4.56-4.45 (m, 2H), 4.28-4.18 (m, 1H), 4.04-3.37 (m, 10H), 2.81-2.71 (m, 1H), 2.17-1.62 (m, 8H), 1.40-1.25 (m, 2H), 1.12-0.97 (m, 6H), 0.89-0.82 (m, 1H), 0.69-0.62 (m, 1H).

Step 3: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide: To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (457 mg, 0.66 mmol) in DMF (5 mL) was added HATU (250.8 mg, 0.66 mmol), DIPEA (255.8 mg, 1.98 mmol) and the mixture stirred at room temperature for 30 min. Ammonium hydroxide (4 mL) were added and the reaction stirred at room temperature for another 2 h. The crude residue obtained was purified by prep-TLC (DCM/MeOH=15/1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (414 mg, 87%) as a white solid. LCMS m/z=692.4 [M+H]⁺; ¹H NMR (400 MHz, DMSO-d₆) δ 8.37 (d, J=11.6 Hz, 1H), 7.84-7.80 (m, 2H), 7.55 (s, 1H), 7.37-7.29 (m, 2H), 7.27-7.14 (m, 5H), 5.34 (s, 2H), 4.50 (s, 2H), 4.34-4.21 (m, 1H), 4.09-3.39 (m, 10H), 2.82-2.72 (m, 1H), 2.17-2.04 (m, 2H), 1.91-1.65 (m, 6H), 1.42-1.25 (m, 1H), 1.12-1.01 (m, 6H), 0.88-0.83 (m, 1H), 0.70-0.63 (m, 1H).

Step 4: 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile: To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (414 mg, 0.59 mmol) in DCM (5 mL) was added N,N-Diethylethanamin (182 mg, 1.79 mmol) and Trifluoroacetic anhydride (188.5 mg, 0.89 mmol). The reaction mixture was stirred at room temperature for 0.5 h. The mixture was purified by prep-TLC (DCM/MeOH=15/1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile (354 mg, 87.8%) as a white solid. LCMS m/z=674.4 [M+H]⁺; ¹H NMR (400 MHz, DMSO-d₆) δ 8.36-8.31 (m, 1H), 7.81 (d, J=10.8 Hz, 1H), 7.59 (t, J=7.8 Hz, 1H), 7.49-7.40 (m, 2H), 7.37-7.30 (m, 2H), 7.22-7.13 (m, 2H), 5.34 (s, 2H), 4.66 (s, 2H), 4.07-3.53 (m, 11H), 3.09-2.99 (m, 1H), 2.21-2.05 (m, 3H), 1.99-1.83 (m, 3H), 1.80-1.69 (m, 2H), 1.38-1.28 (m, 1H), 1.11-0.98 (m, 6H), 0.87-0.80 (m, 1H), 0.68-0.61 (m, 1H).

Step 5: ((S)-8-(((3-(4,4-difluorocyclohexyl)-2-(2H-tetrazol-5-yl)benzyl)oxy)methyl)-2-((S)-2,2-dimethylcyclopropane-1-carbonyl)-2,6-diazaspiro[3.4]octan-6-yl)(1-(4-fluorobenzyl)-1H-pyrazol-4-yl)methanone To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile (150 mg, 0.22 mmol) in toluene (2 mL) was added azidotrimethylsilane (77 mg, 0.07 mmol) and dibutylstannanone (22.2 mg, 0.09 mmol). The reaction mixture was stirred at 150° C. in sealed tube for 6 h. The mixture was diluted with water (20 mL) and extracted with EtOAc (20 mL×3). The combined organic layers were washed with brine, dried over Na₂SO₄, filtered and concentrated. The residue obtained was purified by prep-HPLC to afford ((S)-8-(((3-(4,4-difluorocyclohexyl)-2-(2H-tetrazol-5-yl)benzyl)oxy)methyl)-2-((S)-2,2-dimethylcyclopropane-1-carbonyl)-2,6-diazaspiro[3.4]octan-6-yl)(1-(4-fluorobenzyl)-1H-pyrazol-4-yl)methanone (42.4 mg, 26%) as a yellow solid. LCMS m/z=717.5 [M+H]⁺; ¹H NMR (400 MHz, DMSO-d₆) δ 8.34 (s, 1H), 7.82 (s, 1H), 7.54-7.30 (m, 5H), 7.21-7.14 (m, 2H), 5.35 (d, J=7.6 Hz, 2H), 4.23-3.43 (m, 12H), 2.39-2.27 (m, 1H), 2.16-2.06 (m, 1H), 2.05-1.93 (m, 2H), 1.79-1.58 (m, 6H), 1.38-1.29 (m, 1H), 1.13-0.99 (m, 6H), 0.88-0.82 (m, 1H), 0.68-0.62 (m, 1H).

TABLE 20
The compounds listed in Table 20 were synthesized from (S)-2-(4,4-
difluorocyclohexyl)-6-(((6-(1-(4-fluorobenzyl)-1H-pyrazole-4-carbonyl)-2-(3,3,3-trifluoro-2,2-
dimethylpropanoyl)-2,6-diazaspiro[3.4]octan-8-yl)methoxy)methyl)benzonitrile according to the
procedures outlined for I′-43 using the appropriate commercially available reagents and/or
intermediates described elsewhere.

| Cmpd | | | |
|---|---|---|---|
| No. | Compound | ¹HNMR | LCMS |
| I′-24 | 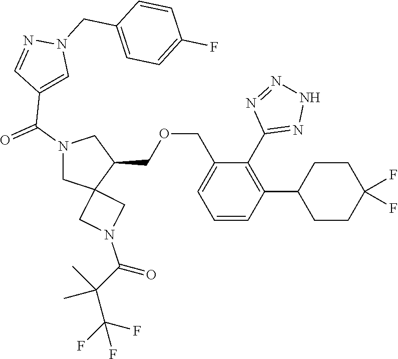 [SMILES: CC(C)(C(=O)N1CC2(CN(C(=O)c3cnn(Cc4ccc(F)cc4)c3)C[C@H]2COCc2cccc(C3CCC(F)(F)CC3)c2-c2nn[nH]n2)C1)C(F)(F)F] | ¹H NMR (400 MHz, Methanol-d4) δ 8.39- 8.16 (m, 1H), 7.90 (s, 1H), 7.41-7.25 (m, 5H), 7.13-7.01 (m, 2H), 5.38 (d, J = 15.5 Hz, 2H), 4.61- 4.46 (m, 2H), 4.32- 4.11 (m, 3H), 4.02-3.61 (m, 5H), 3.53-3.44 (m, 2H), 2.54-2.44 (m, 1H), 2.35-2.23 (m, 1H), 2.07- 1.93 (m, 2H), 1.90- 1.47 (m, 6H), 1.38 (s, 6H). | m/z = 759.3 [M + H]⁺ |

### 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile I′-42

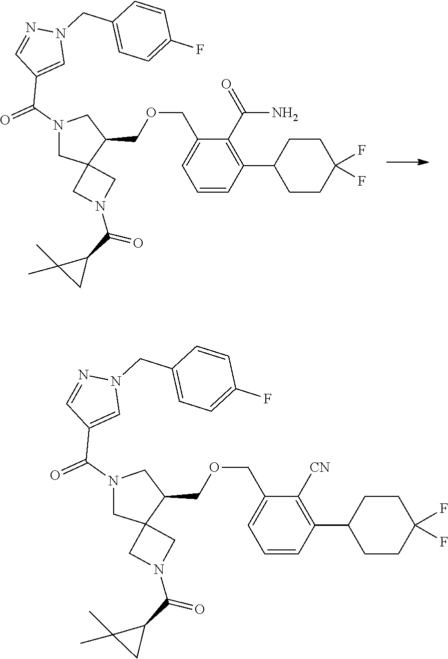 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(N)=O)C1 ; CC1(C)C[C@@H]1C(=O)N1CC2(CN(C(=O)c3cnn(Cc4ccc(F)cc4)c3)C[C@H]2COCc2cccc(C3CCC(F)(F)CC3)c2C#N)C1]

To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzamide (414 mg, 0.59 mmol) in DCM (5 mL) was added N,N-diethylethanamin (182 mg, 1.79 mmol) and trifluoroacetic anhydride (188.5 mg, 0.89 mmol). The reaction mixture was stirred at room temperature for 0.5 h. The mixture was purified by prep-TLC (DCM/MeOH=15/1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzonitrile (354 mg, 87.8%) as a white solid. LCMS m/z=674.4 [M+H]⁺; ¹H NMR (400 MHz, DMSO-d₆) δ 8.36-8.31 (m, 1H), 7.81 (d, J=10.8 Hz, 1H), 7.59 (t, J=7.8 Hz, 1H), 7.49-7.40 (m, 2H), 7.37-7.30 (m, 2H), 7.22-7.13 (m, 2H), 5.34 (s, 2H), 4.66 (s, 2H), 4.07-3.53 (m, 11H), 3.09-2.99 (m, 1H), 2.21-2.05 (m, 3H), 1.99-1.83 (m, 3H), 1.80-1.69 (m, 2H), 1.38-1.28 (m, 1H), 1.11-0.98 (m, 6H), 0.87-0.80 (m, 1H), 0.68-0.61 (m, 1H).

TABLE 21
The compounds listed in Table 21 were synthesized from (S)-2-(4,4-
difluorocyclohexyl)-6-(((6-(1-(4-fluorobenzyl)-1H-pyrazole-4-carbonyl)-2-(3,3,3-trifluoro-2,2-
dimethylpropanoyl)-2,6-diazaspiro[3.4]octan-8-yl)methoxy)methyl)benzamide according to the
procedures outlined for I′-42 using the appropriate commercially available reagents and/or
intermediates described elsewhere.

| Cmpd | | | |
|---|---|---|---|
| No. | Compound | ¹HNMR | LCMS |
| I′-35 | 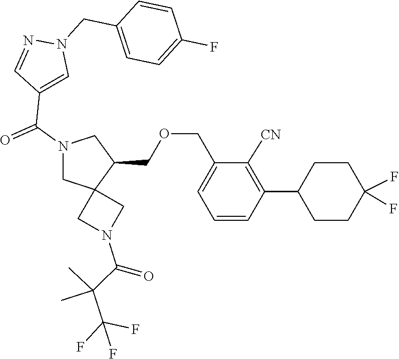 [SMILES: CC(C)(C(=O)N1CC2(CN(C(=O)c3cnn(Cc4ccc(F)cc4)c3)C[C@H]2COCc2cccc(C3CCC(F)(F)CC3)c2C#N)C1)C(F)(F)F] | ¹H NMR (400 MHz, Methanol-d4) δ 8.20 (d, J = 5.6 Hz, 1H), 7.89 (d, J = 5.5 Hz, 1H), 7.60-7.53 (m, 1H), 7.45-7.30 (m, 4H), 7.13-7.04 (m, 2H), 5.35 (s, 2H), 4.68 (d, J = 5.2 Hz, 2H), 4.61-3.52 (m, 10H), 3.16-3.03 (m, 1H), 2.77-2.60 (m, 1H), 2.23-2.13 (m, 2H), 2.06- 1.75 (m, 6H), 1.38 (d, J = 9.4 Hz, 6H). | m/z = 716.3 [M + H]⁺ |

### (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-N-(methylsulfonyl)benzamide I′-44

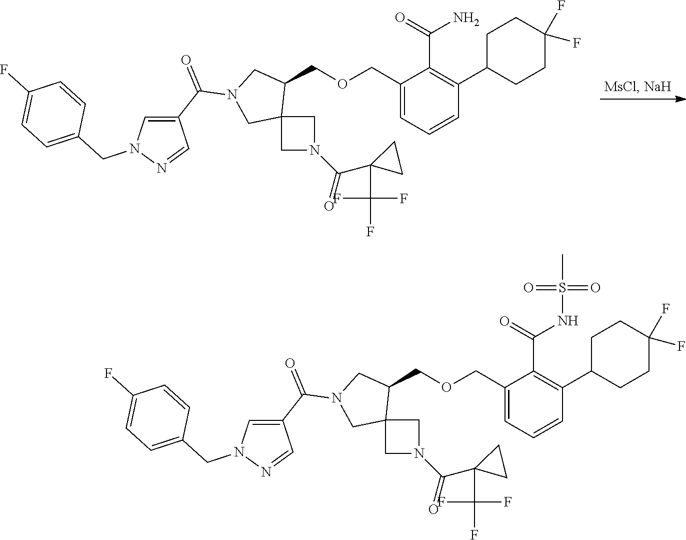 [SMILES: NC(=O)c1c(COC[C@@H]2CN(C(=O)c3cnn(Cc4ccc(F)cc4)c3)CC23CN(C(=O)C2(C(F)(F)F)CC2)C3)cccc1C1CCC(F)(F)CC1 ; CS(=O)(=O)NC(=O)c1c(COC[C@@H]2CN(C(=O)c3cnn(Cc4ccc(F)cc4)c3)CC23CN(C(=O)C2(C(F)(F)F)CC2)C3)cccc1C1CCC(F)(F)CC1]

To a solution of (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzamide (40 mg, 0.055 mmol) in dry THE (1 mL) were added dimethylsulfamoyl chloride (7 mg, 0.066 mmol) and NaH (3 mg, 0.14 mmol) at 0° C. under N₂ atmosphere and the reaction stirred at room temperature overnight. The mixture was diluted with water (50 mL), extracted with EtOAc (10 mL×3) and the combined organic layers washed with brine, dried over Na₂SO₄ and filtered. The solvent was removed and the residue purified by prep-TLC (eluent: DCM:MeOH=15:1) to afford the (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)-N-(methylsulfonyl)benzamide (4.3 mg) as a white solid. LCMS m/z=810.5 [M+H]⁺; ¹H NMR (400 MHz, CD₃OD) δ 8.32-8.21 (m, 1H), 7.93 (s, 1H), 7.36-7.28 (m, 4H), 7.26-7.21 (m, 1H), 7.11-7.04 (m, 2H), 5.36 (s, 2H), 4.60 (s, 2H), 4.37-3.61 (m, 12H), 2.91-2.62 (m, 2H), 2.16-1.77 (m, 9H), 1.20 (s, 4H).

### 2-morpholinoethyl 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoate I′-36

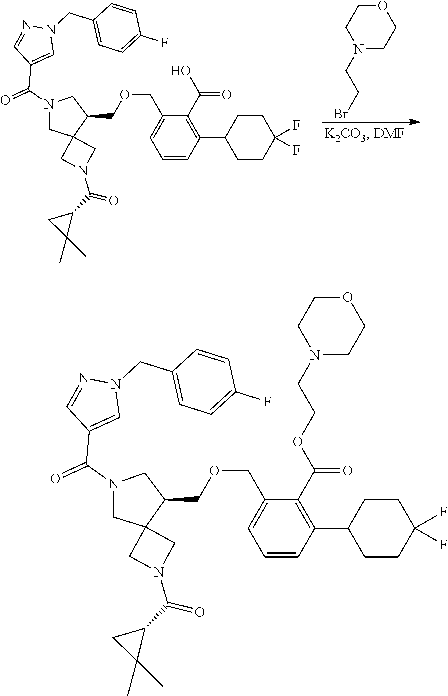 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)O)C1 ; BrCCN1CCOCC1 ; CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)OCCN2CCOCC2)C1]

To a solution of 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)benzoic acid (20 mg, 0.029 mmol) in DMF (1 mL) were added K₂CO₃ (8 mg, 0.058 mmol), 4-(2-bromoethyl)morpholine (7 mg, 0.035 mmol) and KI (Cat.) and the reaction stirred at room temperature overnight. The mixture was diluted with water (50 mL), extracted with EtOAc (10 mL×3) and the combined organic layers washed with brine, dried over Na₂SO₄ and filtered. The solvent was removed and the residue purified by prep-TLC (eluent: DCM:MeOH=15:1) to afford the tert-butyl (S)-2-(4,4-difluorocyclohexyl)-6-(((6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)methoxy)methyl)benzoate (6.6 mg, 28%) as a white solid. LCMS m/z=806.5 [M+H]⁺; ¹H NMR (400 MHz, DMSO-d₆) δ 8.34 (d, J=6.6 Hz, 1H), 7.82 (d, J=9.6 Hz, 1H), 7.37-7.30 (m, 4H), 7.28-7.14 (m, 3H), 5.34 (s, 2H), 4.51 (s, 2H), 4.42-4.32 (m, 2H), 4.22-3.39 (m, 16H), 2.63-2.59 (m, 1H), 2.44-2.36 (m, 4H), 2.14-1.66 (m, 9H), 1.37-1.28 (m, 1H), 1.11-0.98 (m, 6H), 0.87-0.82 (m, 1H), 0.68-0.61 (m, 1H).

TABLE 22
The compounds listed in Table 23 were synthesized from (S)-2-(4,4-
difluorocyclohexyl)-6-(((6-(1-(4-fluorobenzyl)-1H-pyrazole-4-carbonyl)-2-(3,3,3-trifluoro-2,2-
dimethylpropanoyl)-2,6-diazaspiro[3.4]octan-8-yl)methoxy)methyl)benzoic acid according to the
procedures outlined for I′-36 using the appropriate commercially available reagents and/or
intermediates described elsewhere.

| Cmpd. | | | |
|---|---|---|---|
| No. | Compound | ¹HNMR | LCMS |
| I′-31 | 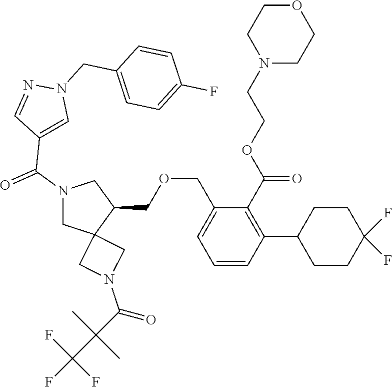 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)OCCN2CCOCC2)C1)C(F)(F)F] | ¹H NMR (400 MHz, DMSO-d6) δ 8.34-8.31 (m, 1H), 7.81 (d, J = 10.2 Hz, 1H), 7.38-7.30 (m, 4H), 7.27-7.14 (m, 3H), 5.34 (s, 2H), 4.51 (s, 2H), 4.41-3.42 (m, 17H), 2.69-2.57 (m, 5H), 2.41- 2.32 (m, 4H), 2.15- 2.06 (m, 2H), 1.80 (s, 2H), 1.75-1.66 (m, 2H), 1.32-1.26 (m, 6H). | m/z = 848.5 [M + H]⁺ |
| I′-30 | 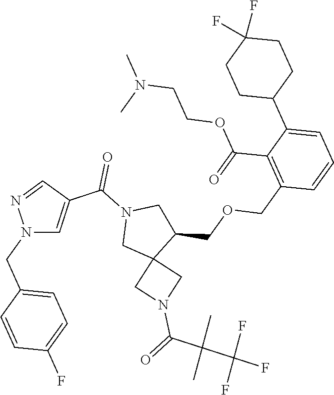 [SMILES: CN(C)CCOC(=O)c1c(COC[C@@H]2CN(C(=O)c3cnn(Cc4ccc(F)cc4)c3)CC23CN(C(=O)C(C)(C)C(F)(F)F)C3)cccc1C1CCC(F)(F)CC1] | ¹H NMR (400 MHz, Methanol-d4) δ 8.21 (d, J = 12.2 Hz, 1H), 7.92 (d, J = 11.4 Hz, 1H), 7.40- 7.29 (m, 4H), 7.22 (t, J = 7.8 Hz, 1H), 7.07 (t, J = 8.6 Hz, 2H), 5.38-5.34 (m, 2H), 4.62-3.46 (m, 14H), 2.81-2.53 (m, 4H), 2.27 (d, J = 6.6 Hz, 6H), 2.18-2.07 (m, 2H), 1.95-1.74 (m, 6H), 1.42- 1.31 (m, 6H). | m/z = 806.5 [M + H]⁺ |

### 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)benzoic acid I′-30

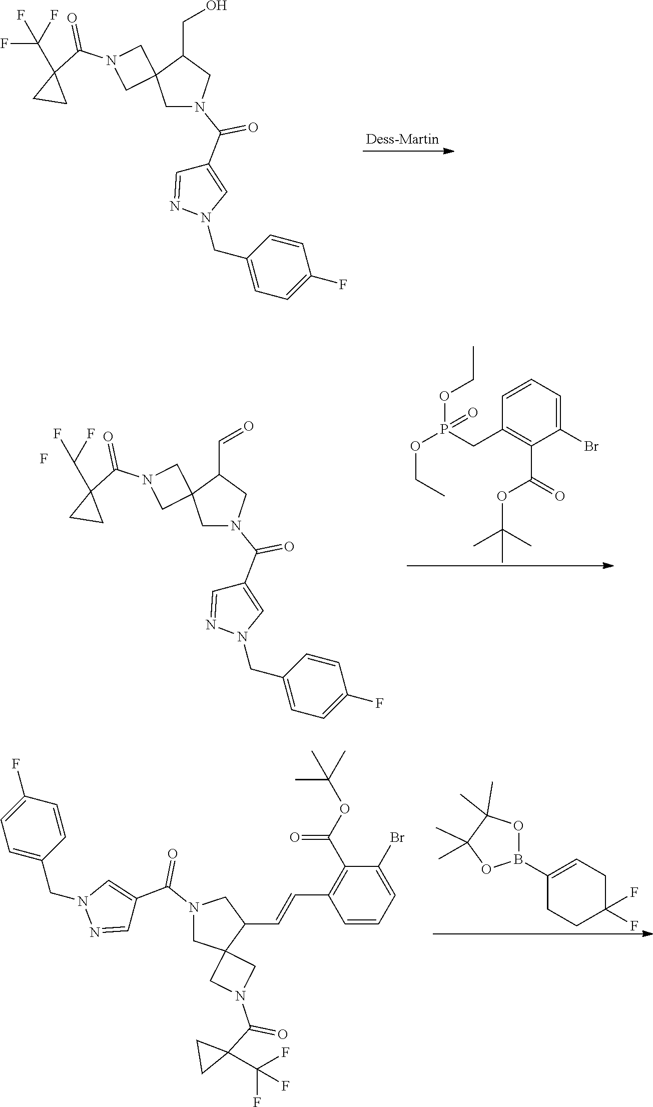

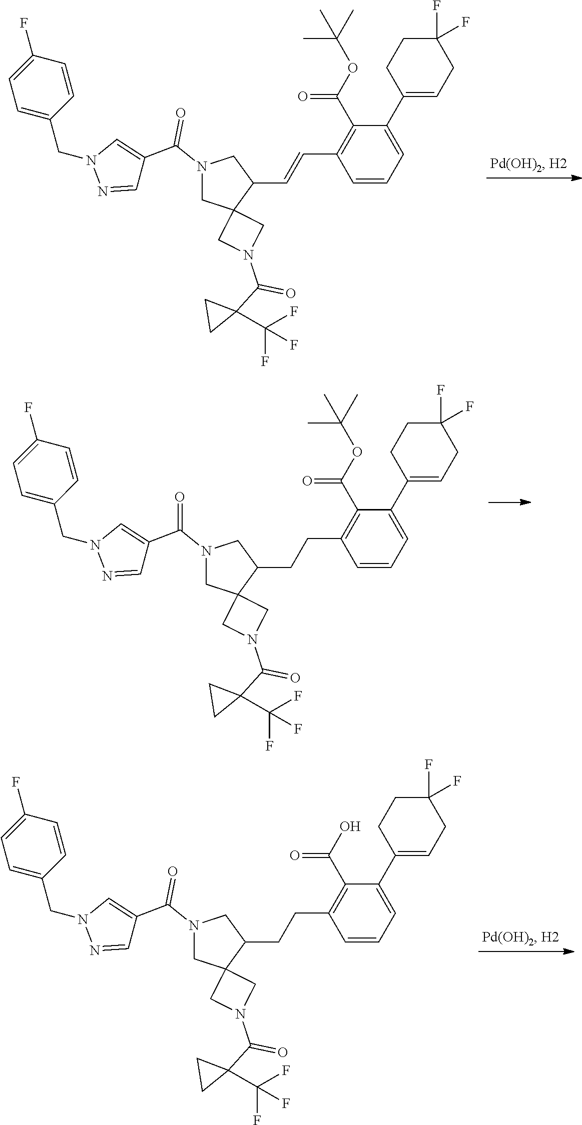 [SMILES: CC(C)(C)OC(=O)c1c(/C=C/C2CN(C(=O)c3cnn(Cc4ccc(F)cc4)c3)CC23CN(C(=O)C2(C(F)(F)F)CC2)C3)cccc1C1=CCC(F)(F)CC1 ; CC(C)(C)OC(=O)c1c(CCC2CN(C(=O)c3cnn(Cc4ccc(F)cc4)c3)CC23CN(C(=O)C2(C(F)(F)F)CC2)C3)cccc1C1=CCC(F)(F)CC1 ; O=C(O)c1c(CCC2CN(C(=O)c3cnn(Cc4ccc(F)cc4)c3)CC23CN(C(=O)C2(C(F)(F)F)CC2)C3)cccc1C1=CCC(F)(F)CC1]

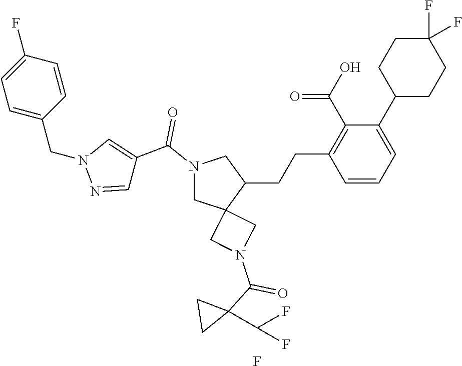

Step 1: 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde To a solution of (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (600 mg, 1.3 mmol) in DCM (6 mL) was added Dess Martin reagent (795 mg, 1.87 mmol) and the mixture stirred at room temperature for 2 h. The reaction mixture was diluted with DCM and washed with a mixture of saturated aqueous NaHCO₃ and saturated aqueous Na₂S₂O₃, water and dried over Na₂SO₄. The solvent was removed to afford 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde (947 mg, 100%) as a colorless oil. LCMS m/z=479.1[M+H]⁺; ¹H NMR (400 MHz, DMSO-d₆) δ 9.73 (s, 1H), 8.35 (s, 1H), 7.83 (s, 1H), 7.33 (t, J=5.2 Hz, 2H), 7.25-7.17 (m, 2H), 5.34 (s, 2H), 4.03 (d, J=7.2 Hz, 4H), 3.85 (dd, J=21.2, 7.6 Hz, 2H), 3.59-3.40 (m, 2H), 1.99 (s, 1H), 1.16 (d, J=7.2 Hz, 4H).

Step 2: tert-butyl (E)-2-bromo-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)benzoate To a solution of tert-butyl 2-bromo-6-((diethoxyphosphoryl)methyl)benzoate (116 mg, 0.2 mmol) in anhydrous THE (1.5 mL) at 0° C. under a N₂ atmosphere was added NaHMDS (2.0 M, 0.1 mL, 1.1 mmol) and 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde (204 mg, 0.4 mmol). The reaction mixture was stirred at room temperature for 2 h. The mixture was diluted with EtOAc (20 mL), quenched with sat. NH₄Cl (15 mL), extracted with EtOAc (20 mL×2), dried over Na₂SO₄, filtered and concentrated. The residue was purified by prep-TLC (eluent: DCM/MeOH=20/1, v/v) to afford tert-butyl (E)-2-bromo-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)benzoate (87 mg, 41%) as a white solid. LCMS m/z=731.3[M+H]⁺; ¹H NMR (400 MHz, DMSO-d₆) δ 8.35 (s, 1H), 7.83 (d, J=12.2 Hz, 1H), 7.67 (s, 1H), 7.56 (d, J=8.0 Hz, 1H), 7.39-7.28 (m, 3H), 7.22-7.13 (m, 2H), 6.48 (s, 1H), 5.36-5.31 (m, 2H), 4.32-3.46 (m, 7H), 3.12 (d, J=39.5 Hz, 1H), 2.01-1.94 (m, 1H), 1.61-1.38 (m, 9H), 1.29 (s, 2H), 1.13 (s, 2H).

Step 3: tert-butyl (E)-4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)-2′,3′,4′,5′-tetrahydro-[1,1′-biphenyl]-2-carboxylate To a solution of tert-butyl (E)-2-bromo-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)benzoate (125 mg, 0.1 mmol) in a mixture of dioxane (1.5 mL) and water (0.5 mL) was added 2-(4,4-difluorocyclohex-1-en-1-yl)-4,4,5,5-tetramethyl-1,3,2-dioxaborolane (45 mg, 0.1 mmol), K₂CO₃ (47 mg, 0.2 mmol) and Pd(pph₃)₄ (20 mg, 0.1 mmol). The mixture was heated at 100° C. for 3 h under N₂ then cooled to room temperature, diluted with water (50 mL) and extracted with EtOAc (20 mL×3). The combined organic layers were washed with brine, dried over Na₂SO₄ and concentrated. The mixture was purified by prep-TLC (eluent: DCM/MeOH=20/1) to afford tert-butyl (E)-4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)-2′,3′,4′,5′-tetrahydro-[1,1′-biphenyl]-2-carboxylate (48 mg, 47%) as a yellow oil. LCMS m/z=769.4[M+H]⁻; ¹H NMR (400 MHz, DMSO-d₆) δ 8.35 (s, 1H), 7.83 (d, J=13.4 Hz, 1H), 7.61-7.13 (m, 6H), 6.66-6.33 (m, 1H), 5.34 (d, J=8.0 Hz, 2H), 4.34-3.42 (m, 8H), 3.05 (d, J=7.6 Hz, 2H), 2.62 (s, 1H), 2.24-1.88 (m, 4H), 1.44 (d, J=13.2 Hz, 9H), 1.10 (d, J=25.1 Hz, 4H).

Step 4: tert-butyl 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylate To a solution of tert-butyl (E)-4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)vinyl)-2′,3′,4′,5′-tetrahydro-[1,1′-biphenyl]-2-carboxylate (48 mg, 0.06 mmol) in MeOH (2 mL) was added Pd(OH)₂ (20 mg) and the mixture stirred overnight under hydrogen at 100° C. The mixture was filtered through celite, the filter cake washed with MeOH and the filtrate concentrated in vacuo to afford tert-butyl 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylate (47 mg, 100%) as a colorless oil. LCMS m/z=771.3[M+H]⁻; ¹H NMR (400 MHz, DMSO-d₆) δ 8.29 (d, J=20.1 Hz, 1H), 7.78 (d, J=32.7 Hz, 1H), 7.36-7.29 (m, 3H), 7.26 (d, J=7.6 Hz, 1H), 7.17 (ddd, J=20.8, 10.6, 6.6 Hz, 3H), 5.40 (s, 1H), 5.34 (s, 2H), 4.26-3.60 (m, 8H), 2.60 (s, 2H), 2.13 (dd, J=13.8, 6.8 Hz, 3H), 1.99 (s, 3H), 1.45 (s, 7H), 1.36 (s, 3H), 1.30 (s, 2H), 1.19-1.12 (m, 4H).

Step 5: 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylic acid To a solution of tert-butyl 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylate (45 mg, 0.05 mmol) in DCM (1 mL) was added TFA (0.5 mL). The mixture was stirred at room temperature for 1 h and then concentrated. The mixture was purified by prep-TLC (eluent: DCM/MeOH=10:1) to afford 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylic acid (20 mg, 48%) as a white solid. LCMS m/z=715.4[M+H]⁻; ¹H NMR (400 MHz, DMSO-d₆) δ 8.36 (d, J=21.6 Hz, 1H), 7.80-7.78 (m, 1H), 7.36-7.32 (m, 2H), 7.21-7.07 (m, 4H), 6.96 (s, 1H), 5.50 (s, 1H), 5.34 (s, 2H), 4.38-3.34 (m, 9H), 2.78-2.51 (m, 7H), 2.06 (dd, J=13.2, 6.6 Hz, 3H), 1.14 (s, 4H).

Step 6: 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)benzoic acid I′-30 To a solution of 4′,4′-difluoro-3-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)-2′,3′,4′,5′-tetrahydro-[1,1′-biphenyl]-2-carboxylic acid (20 mg, 0.02 mmol) in MeOH (1 mL) was added Pd(OH)₂ (10 mg) and the mixture stirred overnight under hydrogen at 50° C. The mixture was filtered through celite, the filter cake washed with MeOH and the filtrate concentrated in vacuo to afford 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethyl)benzoic acid (7.2 mg, 36%) as a white solid. LCMS m/z=717.3[M+H]⁺; ¹H NMR (400 MHz, Chloroform-d) δ 7.85 (d, J=28.5 Hz, 2H), 7.32 (t, J=7.7 Hz, 1H), 7.19 (d, J=7.9 Hz, 3H), 7.05 (d, J=8.3 Hz, 3H), 5.29 (s, 2H), 4.25-3.58 (m, 8H), 2.83 (s, 1H), 2.71 (s, 3H), 2.16 (s, 2H), 1.93-1.72 (m, 8H), 1.22 (s, 4H).

### 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethoxy)benzoic acid I′-32

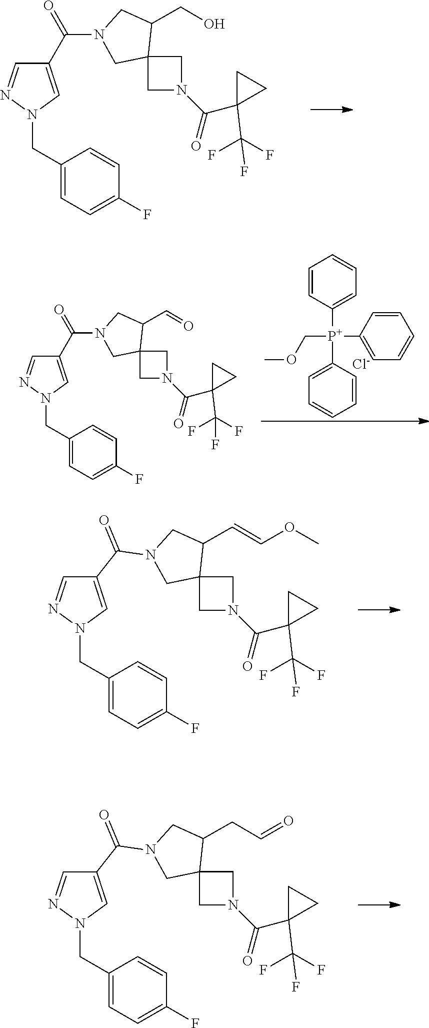

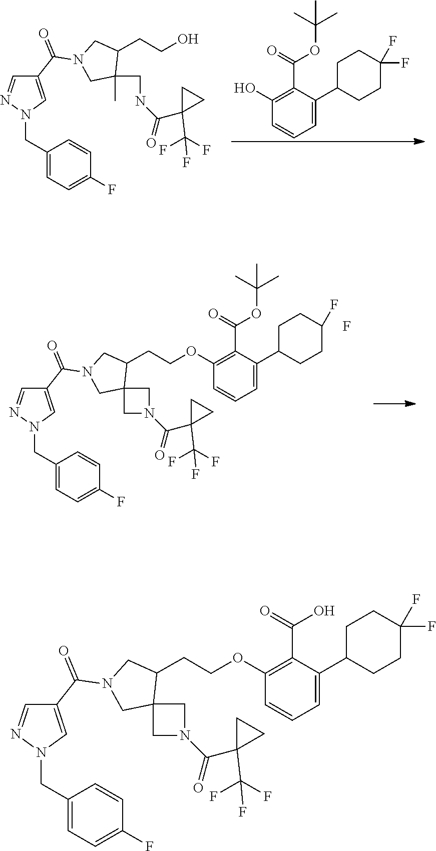

Step 1: 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde To a solution of (1-(4-fluorobenzyl)-1H-pyrazol-4-yl)(8-(hydroxymethyl)-2-(1-(trifluoromethyl)cyclopropane-1-carbonyl)-2,6-diazaspiro[3.4]octan-6-yl)methanone (500 mg, 1.04 mmol) in DCM (6 mL) was added Dess-Martin (600 mg, 1.56 mmol) at 0° C. The reaction mixture was stirred at room temperature for 2 h. The mixture was quenched with Na₂S₂O₃ (6 mL) and extracted with EtOAc (40 mL×2). The combined organic layers were washed with brine, dried over Na₂SO₄, filtered and concentrated to afford 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde (498 mg) which was used directly in the next step. LCMS m/z=477.1 [M−H]⁻; ¹H NMR (DMSO, 400 MHz) δ 8.37-8.29 (m, 1H), 7.82 (d, J=9.2 Hz, 1H), 7.36-7.28 (m, 2H), 7.18 (t, J=8.4 Hz, 2H), 6.70-6.58 (m, 1H), 5.34 (d, J=2.4 Hz, 2H), 4.77 (s, 1H), 4.37-4.21 (m, 1H), 4.06 (s, 2H), 3.92-3.78 (m, 3H), 3.59 (d, 2H), 3.47 (s, 3H), 3.28-3.14 (m, 1H), 2.85-2.65 (m, 1H), 1.26-1.13 (m, 6H).

Step 2: (E)-(1-(4-fluorobenzyl)-1H-pyrazol-4-yl)(8-(2-methoxyvinyl)-2-(1-(trifluoromethyl)cyclopropane-1-carbonyl)-2,6-diazaspiro[3.4]octan-6-yl)methanone To a solution of (methoxymethyl)triphenylphosphonium (463 mg, 1.35 mmol) in dry THE (5 mL) was added NaHMDS (2 mL) at 0° C. The reaction mixture was stirred for 0.5 h at 0° C., 6-(1-(4-fluorobenzyl)-1H-pyrazole-4-carbonyl)-2-(1-(trifluoromethyl)cyclopropane-1-carbonyl)-2,6-diazaspiro[3.4]octane-8-carbaldehyde (498 mg, 1.04 mmoL) was added and heated up to room temperature for another 2 h. The mixture was diluted with water (40 mL) and extracted with EtOAc (40 mL×2). The combined organic layers were washed with brine, dried over Na₂SO₄, filtered and concentrated. The residue obtained was purified by prep-TLC (DCM:MeOH=20:1) to afford (E)-(1-(4-fluorobenzyl)-1H-pyrazol-4-yl)(8-(2-methoxyvinyl)-2-(1-(trifluoromethyl)cyclopropane-1-carbonyl)-2,6-diazaspiro[3.4]octan-6-yl)methanone (182 mg, 35.7%) as a yellow oil. ¹H NMR (DMSO, 400 MHz) δ 9.72 (d, J=9.8 Hz, 1H), 8.36-8.27 (m, 1H), 7.85-7.75 (m, 1H), 7.33 (dd, J=8.4, 5.5 Hz, 2H), 7.18 (t, J=8.6 Hz, 2H), 5.34 (s, 2H), 4.32-4.07 (m, 2H), 3.98-3.49 (m, 5H), 3.40-3.33 (m, 1H), 2.85-2.74 (m, 1H), 2.59 (d, J=11.4 Hz, 1H), 1.17 (t, J=7.2 Hz, 4H)_{∘}

Step 3: 2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)acetaldehyde To a solution of (E)-(1-(4-fluorobenzyl)-1H-pyrazol-4-yl)(8-(2-methoxyvinyl)-2-(1-(trifluoromethyl)cyclopropane-1-carbonyl)-2,6-diazaspiro[3.4]octan-6-yl)methanone (180 mg, 0.36 mmol) in THE (4 mL) was added 1N HCl (3 mL). The reaction mixture was stirred at 50° C. for 4 h. The mixture was diluted with water (30 mL) and extracted with EtOAc (35 mL×2). The combined organic layers were washed with brine, dried over Na₂SO₄, filtered and concentrated to afford 2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)acetaldehyde (182 mg) which was used directly in the next step. LCMS m/z=493.1 [M+H]⁺; ¹H NMR (DMSO, 400 MHz) δ 7.34 (d, J=7.8 Hz, 1H), 7.28-7.25 (m, 2H), 3.95-3.88 (m, 4H), 3.22-3.16 (m, 2H), 2.69-2.61 (m, 1H), 2.14 (s, 2H), 1.89-1.82 (m, 4H), 1.70 (d, J=12.4 Hz, 2H), 1.57 (s, 9H), 1.14 (t, J=7.2 Hz, 6H).

Step 4: (1-(4-fluorobenzyl)-1H-pyrazol-4-yl)(8-(2-hydroxyethyl)-2-(1-(trifluoromethyl)cyclopropane-1-carbonyl)-2,6-diazaspiro[3.4]octan-6-yl)methanone To a solution of 2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)acetaldehyde (110 mg, 0.22 mmol) in MeOH (1 mL) was added NaBH₄ (12.7 mg, 0.34 mmol). The reaction was stirred at room temperature for 1 h. The mixture was diluted with water (15 mL) and extracted with EtOAc (15 mL). The combined organic layers were dried over Na₂SO₄, filtered and concentrated. The residue was purified by pre-TLC (eluent: DCM/MeOH=18:1) to afford (1-(4-fluorobenzyl)-1H-pyrazol-4-yl)(8-(2-hydroxyethyl)-2-(1-(trifluoromethyl)cyclopropane-1-carbonyl)-2,6-diazaspiro[3.4]octan-6-yl)methanone (90 mg, 81.8% yield) as a white solid. LCMS m/z=495.4 [M+H]⁺; ¹H NMR (400 MHz, Chloroform-d) δ 7.82 (m, 2H), 7.25-7.20 (m, 2H), 7.09-7.02 (m, 2H), 5.28 (s, 2H), 4.42-3.68 (m, 10H), 3.46 (m, 1H), 2.37 (s, 1H), 1.89 (s, 1H), 1.22 (s, 4H).

Step 5: tert-butyl 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethoxy)benzoate: To a solution of (1-(4-fluorobenzyl)-1H-pyrazol-4-yl)(8-(2-hydroxyethyl)-2-(1-(trifluoromethyl)cyclopropane-1-carbonyl)-2,6-diazaspiro[3.4]octan-6-yl)methanone (80 mg, 0.16 mmol) and tert-butyl 2-(4,4-difluorocyclohexyl)-6-hydroxybenzoatein (61 mg, 0.19 mmol) in toluene (1 mL) was added DIAD (131 mg, 0.65 mmol) and PPh₃ (170 mg, 0.65 mmol). The mixture was stirred at 130° C. for 1 h. The mixture was diluted with water (20 mL) and extracted with EtOAc (20 mL×2). The combined organic layers were washed with brine, dried over Na₂SO₄, filtered and concentrated. The residue obtained was purified by pre-TLC (eluent: DCM:MeOH=10:1) to afford tert-butyl 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8- yl)ethoxy)benzoate (12 mg, 9% yield) as a yellow solid. LCMS m/z=789.3 [M+H]⁺.

Step 6: 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethoxy)benzoic acid I′-32: To a solution of tert-butyl 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethoxy)benzoate (12 mg, 0.02 mmol) in DCM (2 mL) was added TFA (1 mL). The reaction mixture was stirred at room temperature for 1 h. The solvent was removed under vacuum and purified by prep-HPLC to afford 2-(4,4-difluorocyclohexyl)-6-(2-(6-(1-(4-fluorobenzyl)-1H-pyrazole-4-carbonyl)-2-(1-(trifluoromethyl)cyclopropane-1-carbonyl)-2,6-diazaspiro[3.4]octan-8-yl)ethoxy)benzoic acid (4 mg, 36% yield) as a white solid. LCMS m/z=733.4 [M+H]⁺; ¹H NMR (400 MHz, Methanol-d₄) δ 8.39 (d, J=38.0 Hz, 1H), 7.97 (d, J=36.8 Hz, 1H), 7.34-7.21 (m, 3H), 7.09-7.03 (m, 1H), 7.02-6.95 (m, 1H), 6.93-6.83 (m, 2H), 5.41-5.33 (m, 2H), 4.38-3.49 (m, 10H), 2.77 (s, 1H), 2.61-2.36 (m, 1H), 2.16-2.02 (m, 3H), 1.97-1.70 (m, 7H), 1.24 (s, 4H).

### 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N-(2-morpholinoethyl)benzamide I′-25

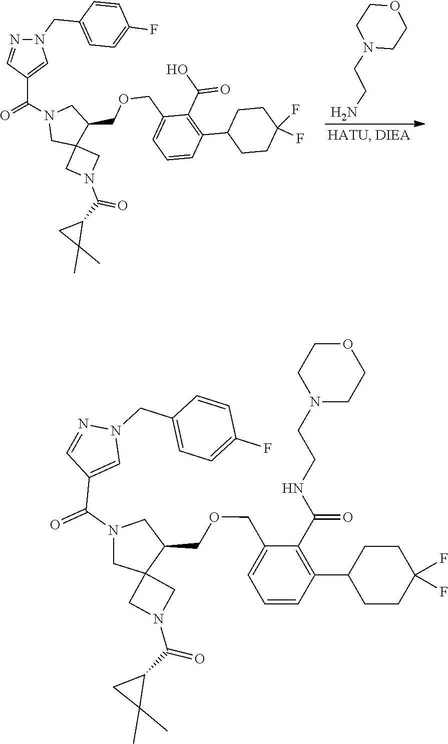 [SMILES: CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)O)C1 ; NCCN1CCOCC1 ; CC1(C)C[C@@H]1C(=O)N1CC2(CN(C(=O)c3cnn(Cc4ccc(F)cc4)c3)C[C@H]2COCc2cccc(C3CCC(F)(F)CC3)c2C(=O)NCCN2CCOCC2)C1]

2-(4,4-difluorocyclohexyl)-6-((((S)-2-((S)-2,2-dimethylcyclopropane-1carbonyl)-6-(1-(4-fluorobenzyl)-1H-pyrazole-4-carbonyl)-2,6-diazaspiro[3.4]octan-8-yl)methoxy)methyl) benzoic acid (20 mg, 0.028 mmol) in DCM (1 mL) was added 2-morpholinoethan-1-amine (4 mg, 0.028 mmol), HATU (11.2 mg, 0.028 mmol) and DIPEA (8.4 mg, 0.06 mmol, 3.0 eq). The reaction mixture was stirred for 3 h. The mixture was diluted with water (30 mL), extracted with DCM (50 mL×3). The combined organic layers were washed with brine, dried over Na₂SO₄, filtered, concentrated and purified by prep-TLC (eluent: DCM:MeOH=15:1) to afford 2-(4,4-difluorocyclohexyl)-6-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-N-(2-morpholinoethyl)benzamide (11.3 mg, 50%) as a white solid. LCMS m/z=805.4 [M+H]⁺; ¹H NMR (400 MHz, DMSO-d₆) δ 8.35 (s, 1H), 8.27 (s, 1H), 7.83 (d, J=7.4 Hz, 1H), 7.33 (t, J=7.0 Hz, 2H), 7.26-7.14 (m, 4H), 5.34 (s, 2H), 4.47 (s, 2H), 4.27-4.20 (m, 1H), 4.09-3.98 (m, 2H), 3.97-3.91 (m, 1H), 3.87 (d, J=9.2 Hz, 1H), 3.84-3.71 (m, 2H), 3.71-3.61 (m, 3H), 3.55 (s, 6H), 2.67 (s, 1H), 2.47-2.30 (m, 6H), 2.09 (d, J=11.2 Hz, 2H), 1.92-1.59 (m, 7H), 1.35 (s, 2H), 1.07 (d, J=24.5 Hz, 6H), 0.86 (s, 1H), 0.66 (s, 1H).

TABLE 24
The compounds listed in Table 24 were synthesized from (S)-2-(4,4-
difluorocyclohexyl)-6-(((6-(1-(4-fluorobenzyl)-1H-pyrazole-4-carbonyl)-2-(3,3,3-trifluoro-2,2-
dimethylpropanoyl)-2,6-diazaspiro[3.4]octan-8-yl)methoxy)methyl)benzoic acid according to the
procedures outlined for I′-25 using the appropriate commercially available reagents and/or
intermediates described elsewhere.

| Cmpd. | | | |
|---|---|---|---|
| No. | Compound | ¹HNMR | LCMS |
| I′-22 | 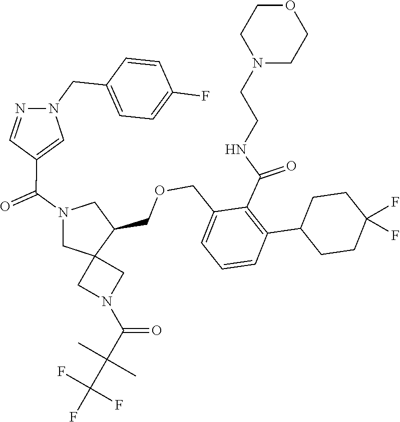 [SMILES: CC(C)(C(=O)N1CC2(CN(C(=O)c3cnn(Cc4ccc(F)cc4)c3)C[C@H]2COCc2cccc(C3CCC(F)(F)CC3)c2C(=O)NCCN2CCOCC2)C1)C(F)(F)F] | ¹H NMR (400 MHz, DMSO-d6) δ 8.36 − 8.24 (m, 2H), 7.82 (d, J = 8.2 Hz, 1H), 7.35 − 7.30 (m, 2H), 7.25 − 7.14 (m, 5H), 5.34 (s, 2H), 4.47 (s, 2H), 4.42 − 4.06 (m, 2H), 3.96 − 3.80 (m, 2H), 3.73 − 3.33 (m, 12H), 2.74 − 2.66 (m, 1H), 2.46 − 2.36 (m, 6H), 2.15 − 2.05 (m, 2H), 1.94 − 1.60 (m, 7H), 1.32 (s, 6H). | m/z = 847.4 [M + H]⁺ |
| I′-18 | 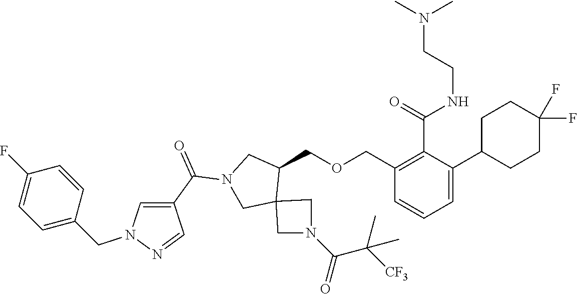 [SMILES: CN(C)CCNC(=O)c1c(COC[C@@H]2CN(C(=O)c3cnn(Cc4ccc(F)cc4)c3)CC23CN(C(=O)C(C)(C)C(F)(F)F)C3)cccc1C1CCC(F)(F)CC1] | 1H NMR (400 MHz, CD3OD) δ 8.19 (s, 1H), 7.92 − 7.88 (m, 1H), 7.36 − 7.23 (m, 5H), 7.11 − 7.05 (m, 2H), 5.35 (s, 2H), 4.55 − 4.45 (m, 2H), 4.36 − 3.81 (m, 5H), 3.77 − 3.57 (m, 6H), 3.42 − 3.36 (m, 2H), 3.03 − 2.99 (m, 6H), 2.71 − 2.54 (m, 2H), 2.18 − 1.78 (m, 9H), 1.37 (s, 6H). | m/z = 805.5 [M + H]⁺ |

### 2-(4,4-difluorocyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoic acid I-117

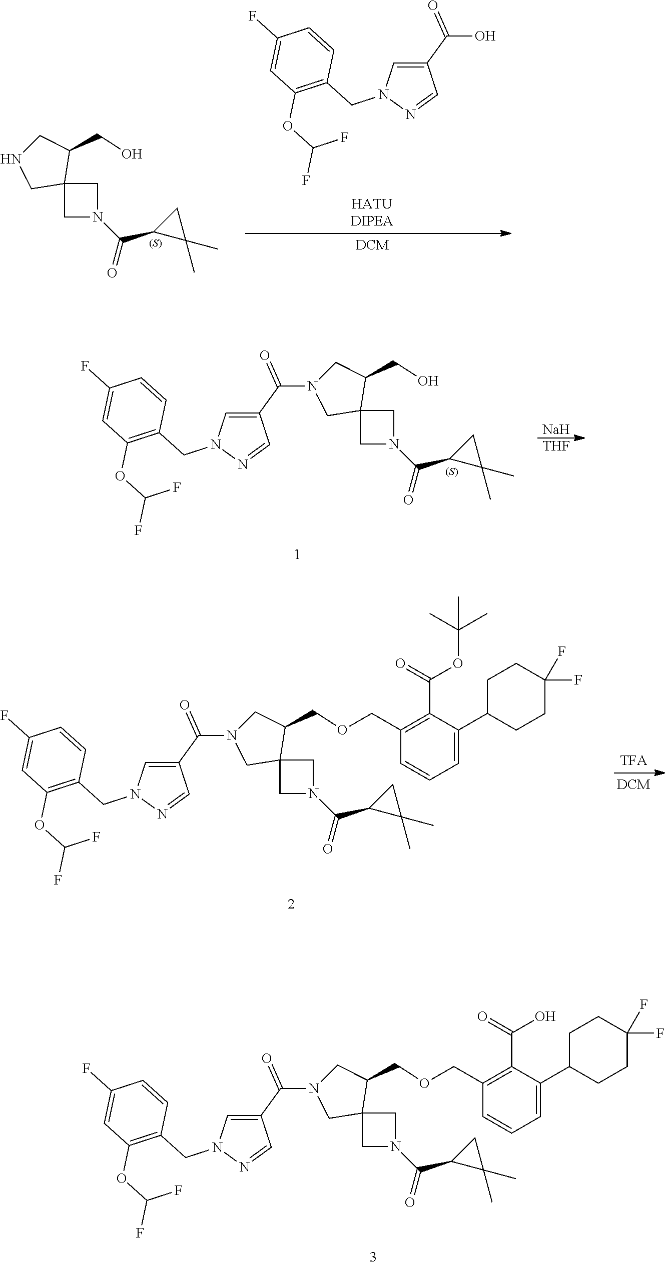

Step 1: (1-(2-(difluoromethoxy)-4-fluorobenzyl)-1H-pyrazol-4-yl)((S)-2-((S)-2,2-dimethylcyclopropane-1-carbonyl)-8-(hydroxymethyl)-2,6-diazaspiro[3.4]octan-6-yl)methanone To a solution of ((S)-2,2-dimethylcyclopropyl)((S)-8-(hydroxymethyl)-2,6-diazaspiro[3.4]octan-2-yl)methanone (70 mg, 0.29 mmol) in DMF (4 mL) was added 1-(2-(difluoromethoxy)-4-fluorobenzyl)-1H-pyrazole-4-carboxylic acid (84 mg, 0.29 mmol), EDCI (83 mg, 0.43 mmol), HOBT (59 mg, 0.43 mmol) and DIPEA (112 mg, 0.87 mmol). The mixture was stirred at room temperature for 4 h. The mixture was diluted with water (15 mL) and extracted with EtOAc (30 mL×2). The combined organic layers were washed with water, brine, dried over Na₂SO₄, filtered and concentrated. The mixture was purified by prep-TLC (eluent: DCM:MeOH=10:1) to afford (1-(2-(difluoromethoxy)-4-fluorobenzyl)-1H-pyrazol-4-yl)((S)-2-((S)-2,2-dimethylcyclopropane-1-carbonyl)-8-(hydroxymethyl)-2,6-diazaspiro[3.4]octan-6-yl)methanone (10 mg, 6.8%) as a white solid. LCMS m/z=507.2 [M+H]⁺.

Step 2: tert-butyl 2-(4,4-difluoro cyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoate To a solution of (1-(2-(difluoromethoxy)-4-fluorobenzyl)-1H-pyrazol-4-yl)((S)-2-((S)-2,2-dimethylcyclopropane-1-carbonyl)-8-(hydroxymethyl)-2,6-diazaspiro[3.4]octan-6-yl)methanone benzoate (10 mg, 0.02 mmol) and tert-butyl 2-(bromomethyl)-6-(4,4-difluorocyclohexyl)benzoate (9 mg, 0.022 mmol) in THF (1 mL) was added 60% NaH (2.6 mg, 0.06 mmol). The mixture was stirred at room temperature overnight. The mixture was diluted with water (10 mL) and extracted with EtOAc (20 mL×2). The combined organic layers were washed with brine, dried over Na₂SO₄, filtered and concentrated. The mixture was purified by prep-TLC (eluent: DCM:MeOH=15:1) to afford tert-butyl 2-(4,4-difluorocyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoate (5 mg, 31%) as a white solid. LCMS m/z=771.4 [M+H]; ¹H NMR (400 MHz, CD₃OD) δ 8.21-8.15 (m, 1H), 7.93-7.88 (m, 1H), 7.37-7.21 (m, 4H), 7.16-6.75 (m, 3H), 5.40 (s, 2H), 4.62-4.52 (m, 2H), 4.41-4.32 (m, 1H), 4.20-3.51 (m, 10H), 2.21-2.10 (m, 2H), 1.92-1.76 (m, 6H), 1.62-1.57 (m, 9H), 1.21-1.05 (m, 7H), 0.80-0.69 (m, 1H).

Step 3: 2-(4,4-difluorocyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoic acid To a solution of tert-butyl 2-(4,4-difluorocyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoate (5 mg, 0.006 mmol) in DCM (1 mL) was added TFA (1 mL). The mixture was stirred at room temperature for 4 h. The mixture was purified by prep-TLC (eluent: DCM:MeOH=15:1) to afford 2-(4,4-difluorocyclohexyl)-6-((((S)-6-(1-(2-(difluoromethoxy)-4-fluorobenzyl)-1H-pyrazole-4-carbonyl)-2-((S)-2,2-dimethylcyclopropane-1-carbonyl)-2,6-diazaspiro[3.4]octan-8-yl)methoxy)methyl)benzoic acid (3.5 mg, 76%) as a white solid. LCMS m/z=759.4 [M+H]⁺; ¹H NMR (400 MHz, CD₃OD) δ 8.26-8.14 (m, 1H), 7.96-7.82 (m, 1H), 7.38-7.20 (m, 4H), 7.07-6.92 (m, 2H), 5.40 (s, 2H), 4.66-4.54 (m, 2H), 4.43-3.55 (m, 10H), 2.89-2.79 (m, 1H), 2.13-2.08 (m, 1H), 1.93-1.79 (m, 4H), 1.32-1.30 (m, 4H), 1.16-1.00 (m, 7H), 0.81-0.67 (m, 1H).

### (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoic acid I′-26

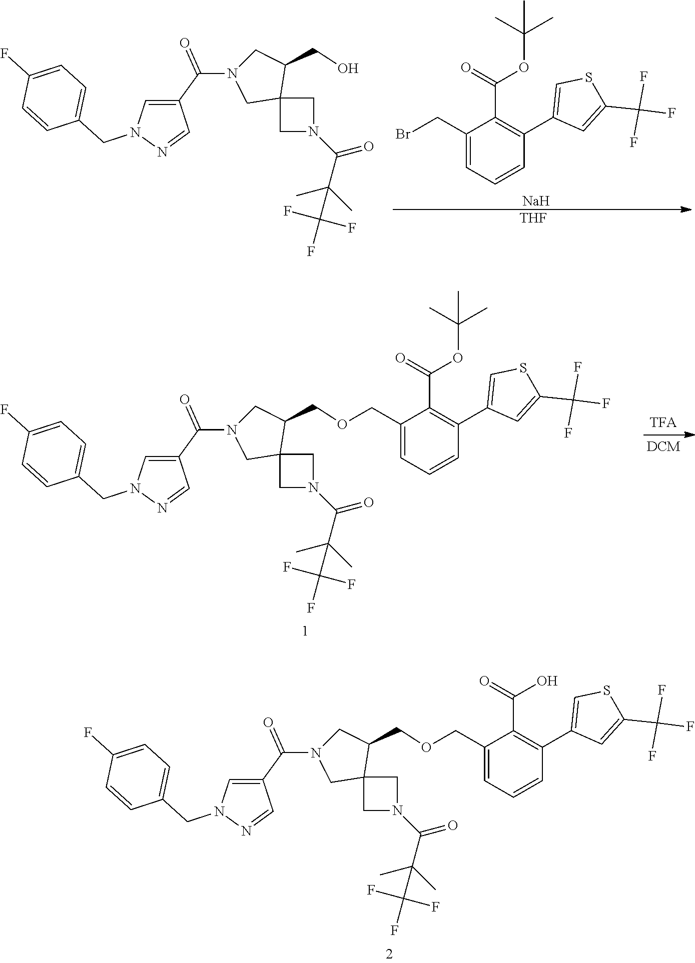

Step 1: tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoate To a solution of tert-butyl 2-(bromomethyl)-6-(5-(trifluoromethyl)thiophen-3-yl)benzoate (38 mg, 0.09 mmol) in anhydrous THE (0.5 mL) at 0° C. was added NaH (60% dispersion in oil, 36 mg, 0.90 mmol) over 10 min. The reaction mixture was stirred at room temperature for 30 min then a solution (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (44 mg, 0.09 mmol) in THF (0.5 mL) was added at 0° C. The reaction was allowed to warm to room temperature and stirred for another 40 min. The reaction was quenched by H₂O (40 mL) and extracted with EtOAc (30 mL×3). The combined organic layers were dried over Na₂SO₄, filtered and concentrated. The residue was purified by prep-TLC (eluent: DCM:MeOH=15:1) to afford tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoate (58 mg, 78%) as a yellow solid. LCMS m/z=823.4 [M+H]⁺.

Step 2: (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoic acid To a solution of tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoate (40 mg, 0.05 mmol) in DCM (0.5 mL) was added TFA (1.0 mL). The reaction mixture was stirred at room temperature for 3 h. The mixture was purified by prep-HPLC to afford (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(5-(trifluoromethyl)thiophen-3-yl)benzoic acid (29 mg, 78%) as a white solid. LCMS m/z=767.2 [M+H]⁺; ¹H NMR (400 MHz, CD₃OD) δ 8.22 (d, J=27.0 Hz, 1H), 7.89 (s, 1H), 7.71 (s, 1H), 7.62 (s, 1H), 7.47-7.40 (m, 3H), 7.35-7.25 (m, 2H), 7.10-7.01 (m, 2H), 5.36-5.26 (m, 2H), 4.69-4.65 (m, 2H), 4.55-4.37 (m, 1H), 4.33-4.20 (m, 1H), 4.10-4.01 (m, 1H), 3.98-3.86 (m, 2H), 3.84-3.47 (m, 5H), 2.68-2.56 (m, 1H), 1.40-1.33 (m, 6H).

### (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylic acid I′-27

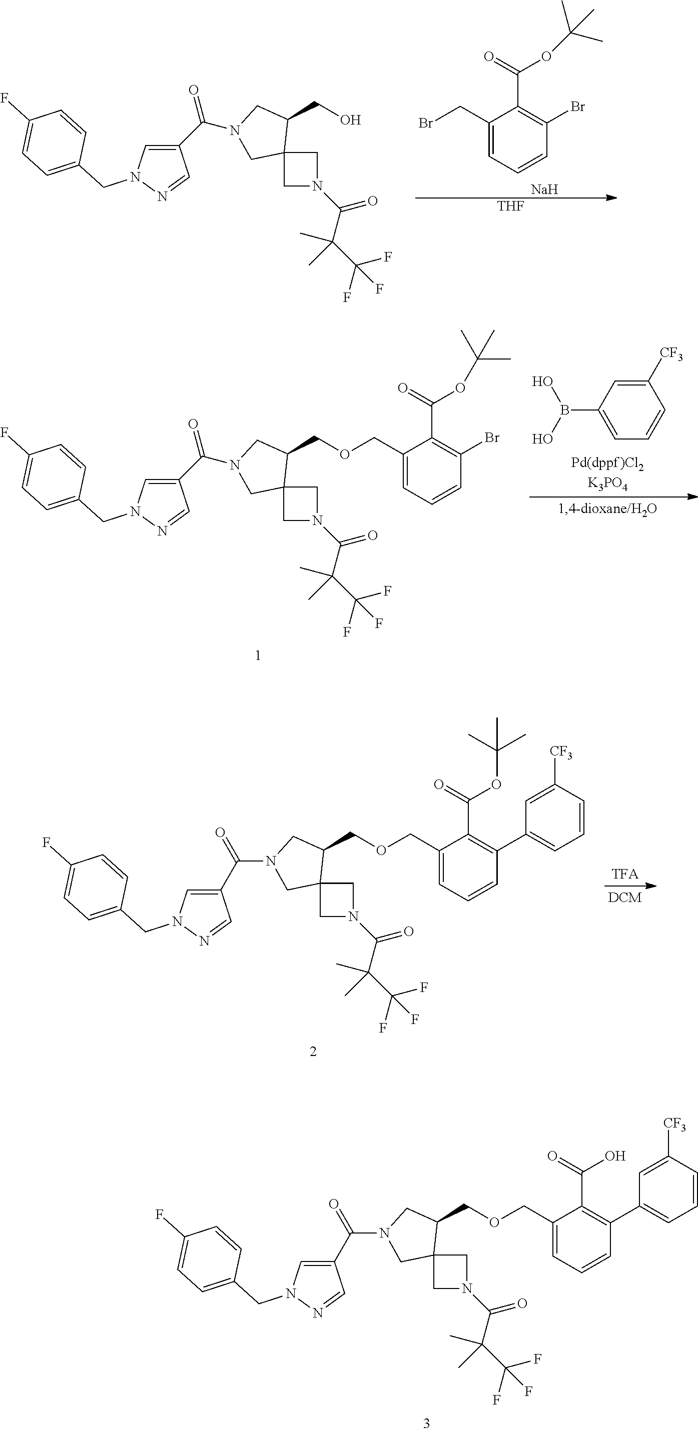

Step 1: tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (90 mg, 0.19 mmol) and tert-butyl 2-bromo-6-(bromomethyl)benzoate (80 mg, 0.23 mmol) in dry THE (1 mL) at 0° C. was added NaH (38 mg, 0.95 mmol). The resulting reaction mixture was stirred overnight. Diluted with water (10 mL) and extracted with EtOAc (30 mL). The combined organic layers were washed with brine, dried over Na₂SO₄, filtered and concentrated. The mixture was purified by prep-TLC (eluent: DCM:MeOH=15:1) to afford tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (165 mg, 75%) as a white solid. LCMS m/z=751.2 [M+H]⁺; ¹HNMR (400 MHz, CD₃OD) δ 8.26-8.17 (m, 1H), 7.94-7.88 (m, 1H), 7.60-7.52 (m, 1H), 7.44-7.19 (m, 4H), 7.13-7.04 (m, 2H), 5.36 (s, 2H), 4.62-3.47 (m, 12H), 2.70-2.56 (m, 1H), 1.63-1.54 (m, 9H), 1.42-1.33 (m, 6H).

Step 2: tert-butyl (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylate To a solution of tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (65 mg, 0.09 mmol) in dioxane (0.8 mL) and H₂O (0.2 mL) was added (3-(trifluoromethyl)phenyl)boronic acid (23 mg, 0.12 mmol), K₃PO₄ (38 mg, 0.18 mmol) and Pd(dppf)Cl₂ (7 mg, 0.01 mmol). The mixture was stirred at 100° C. for 5 h under N₂ atomosphere, Diluted with water (5 mL) and extracted with EtOAc (5 mL×3). The combined organic layers were washed with brine, dried over Na₂SO₄ and concentrated. The mixture was purified by reverse column (70% ACN in water) to afford tert-butyl (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylate (50 mg, 60%) as a white solid. LCMS m/z=817.4 [M+H]⁺; ¹HNMR (400 MHz, CD₃OD) δ 8.20 (s, 1H), 7.91-7.87 (m, 1H), 7.72-7.57 (m, 4H), 7.52-7.42 (m, 2H), 7.36-7.24 (m, 3H), 7.10-7.01 (m, 2H), 5.32 (d, J=26.4 Hz, 2H), 4.67-3.48 (m, 12H), 2.71-2.58 (m, 1H), 1.41-1.33 (m, 6H), 1.25 (s, 9H).

Step 3: (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylic acid To a solution of tert-butyl (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylate (50 mg, 0.06 mmol) in DCM (0.5 mL) was added TFA (0.3 mL). The reaction mixture was stirred at room temperature for 2 h. The solvent was removed and the residue was purified by prep-TLC to afford (S)-3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-3′-(trifluoromethyl)-[1,1′-biphenyl]-2-carboxylic acid (30 mg, 64%) as a white solid. LCMS m/z=761.3 [M+H]⁺; ¹HNMR (400 MHz, CD₃OD) δ 8.24 (d, J=34.3 Hz, 1H), 7.92-7.87 (m, 1H), 7.74-7.54 (m, 4H), 7.47-7.23 (m, 5H), 7.11-7.00 (m, 2H), 5.29 (d, J=39.5 Hz, 2H), 4.73-3.62 (m, 12H), 2.70-2.57 (m, 1H), 1.41-1.34 (m, 6H).

### (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4-(trifluoromethyl)piperidin-1-yl)benzoic acid I′-19

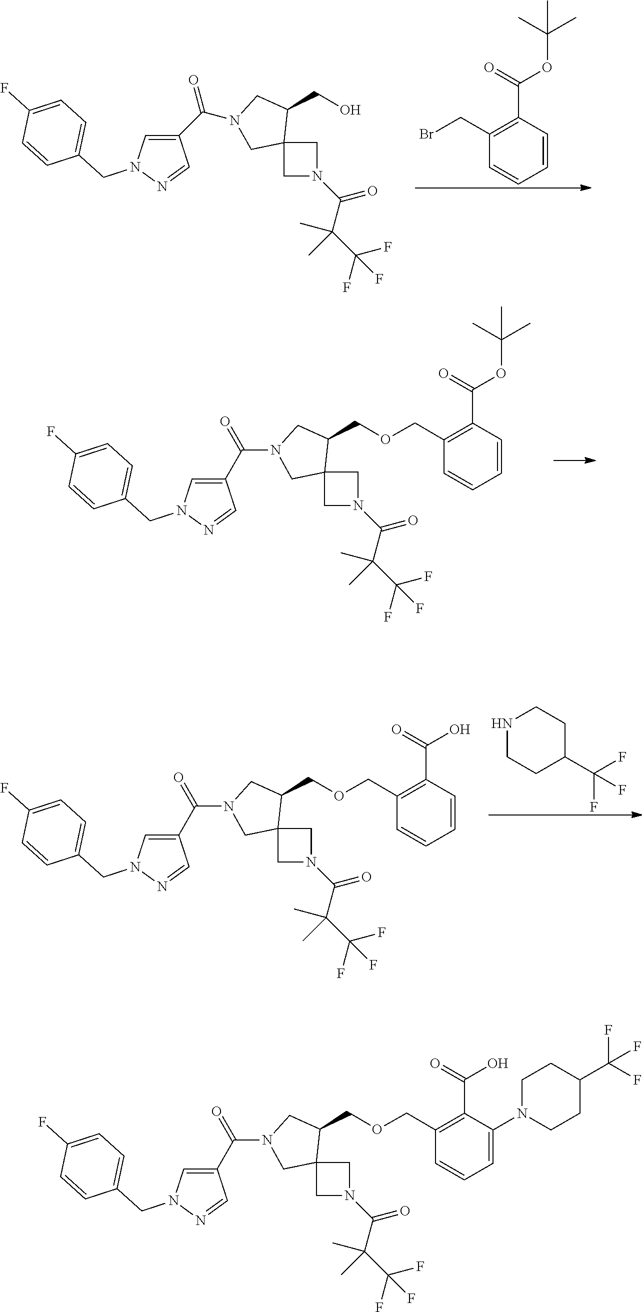 [SMILES: CC(C)(C(=O)N1CC2(CN(C(=O)c3cnn(Cc4ccc(F)cc4)c3)C[C@H]2CO)C1)C(F)(F)F ; CC(C)(C)OC(=O)c1ccccc1CBr ; CC(C)(C)OC(=O)c1ccccc1COC[C@@H]1CN(C(=O)c2cnn(Cc3ccc(F)cc3)c2)CC12CN(C(=O)C(C)(C)C(F)(F)F)C2 ; CC(C)(C(=O)N1CC2(CN(C(=O)c3cnn(Cc4ccc(F)cc4)c3)C[C@H]2COCc2ccccc2C(=O)O)C1)C(F)(F)F ; FC(F)(F)C1CCNCC1 ; CC(C)(C(=O)N1CC2(CN(C(=O)c3cnn(Cc4ccc(F)cc4)c3)C[C@H]2COCc2cccc(N3CCC(C(F)(F)F)CC3)c2C(=O)O)C1)C(F)(F)F]

Step 1: tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate: To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (276 mg, 0.57 mmol) and tert-butyl 2-(bromomethyl)-6-(4,4-difluorocyclohexyl)benzoate (170 mg, 0.63 mmol) in anhydrous THF (5 mL) under N₂ atmosphere at −78° C. was added NaH (114 mg, 2.86 mmol). The resulting mixture was stirred at room temperature for 1 h. The mixture was diluted with water (30 mL) and extracted with EtOAc (30 mL). The combined organic layers were washed with brine, dried over Na₂SO₄, filtered and concentrated. The residue was purified by pre-TLC (eluent: DCM:MeOH=15:1) to afford tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (190 mg, 49% yield) as a white solid. LCMS m/z=673.2 [M+H]⁺; ¹H NMR (400 MHz, DMSO-d₆) δ 8.33 (d, J=10.0 Hz, 1H), 7.82 (d, J=15.6 Hz, 1H), 7.76 (t, J=7.0 Hz, 1H), 7.54-7.42 (m, 2H), 7.40-7.28 (m, 3H), 7.21-7.13 (m, 2H), 5.34 (s, 2H), 4.79 (s, 2H), 4.44-3.36 (m, 10H), 2.68-2.54 (m, 1H), 1.52 (d, J=8.4 Hz, 9H), 1.30 (s, 6H).

Step 2: (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid: To a solution of tert-butyl (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (190 mg, 0.28 mmol) in DCM (2 mL) was added TFA (1 mL). The reaction mixture was stirred at room temperature for 2 h. The solvent was removed under vacuum and purified by pre-TLC to afford (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (150 mg, 86% yield) as a white solid. LCMS m/z=617.3 [M+H]⁺.

Step 3: (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4-(trifluoromethyl)piperidin-1-yl)benzoic acid I′-19: To a solution of 4-(trifluoromethyl)piperidine (22.4 mg, 0.15 mmol) and tert-butyl hypochlorite (16 mg, 0.15 mmol) in Dixoane (1 mL)(mixture A). The mixture was stirred at room temperature for 30 min. (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (75 mg, 0.12 mmol), silver acetate (31 mg, 0.18 mmol) and Bis[(pentamethylcyclopentadienyl)dichloro-rhodium](2.3 mg, 3 mol %) in MeOH (1 mL)(mixture B). The mixture was stirred at room temperature for 30 min. Mixture (A) and (B) was stirred at 60° C. for 16 h. The mixture was diluted with water (30 mL) and extracted with EtOAc (30 mL). The combined organic layers were washed with brine, dried over Na₂SO₄, filtered and concentrated. The residue was purified by prep-HPLC to afford (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(4-(trifluoromethyl)piperidin-1-yl)benzoic acid (16.4 mg, 17.6% yield) as a white solid. LCMS m/z=768.5 [M+H]⁺; ¹H NMR (400 MHz, Methanol-d₄) δ 8.24 (d, J=16.2 Hz, 1H), 7.91 (s, 1H), 7.45-7.29 (m, 5H), 7.07 (td, J=8.8, 6.8 Hz, 2H), 5.36 (s, 2H), 4.77 (d, J=10.0 Hz, 2H), 4.59-3.49 (m, 10H), 3.28 (s, 2H), 2.96 (d, J=12.0 Hz, 2H), 2.74-2.62 (m, 1H), 2.40 (s, 1H), 2.06-1.96 (m, 2H), 1.84-1.69 (m, 2H), 1.38 (d, J=9.6 Hz, 6H).

### (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoic acid I′-14

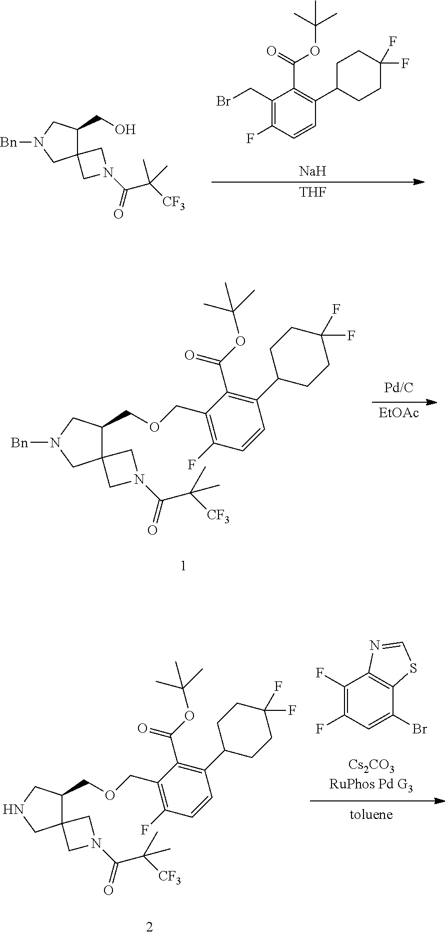

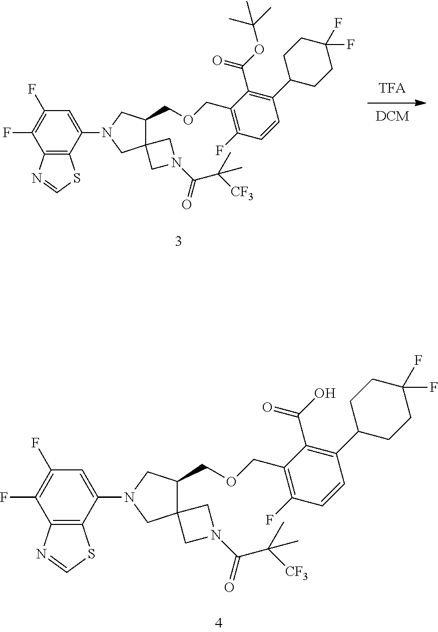 [SMILES: CC(C)(C)OC(=O)c1c(C2CCC(F)(F)CC2)ccc(F)c1COC[C@@H]1CN(c2cc(F)c(F)c3ncsc23)CC12CN(C(=O)C(C)(C)C(F)(F)F)C2 ; CC(C)(C(=O)N1CC2(C1)CN(c1cc(F)c(F)c3ncsc13)C[C@H]2COCc1c(F)ccc(C2CCC(F)(F)CC2)c1C(=O)O)C(F)(F)F]

Step 1: tert-butyl (S)-2-(((6-benzyl-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate: To a solution of (S)-1-(6-benzyl-8-(hydroxymethyl)-2,6-diazaspiro[3.4]octan-2-yl)-3,3,3-trifluoro-2,2-dimethylpropan-1-one (240 mg, 0.6 mmol) in THF (5 mL) at 0° C. was added NaH (129 mg, 3.2 mmol). The reaction was stirred at room temperature for 10 minutes. tert-butyl 2-(bromomethyl)-6-(4,4-difluorocyclohexyl)-3-fluorobenzoate (290 mg, 0.7 mmol) was added to the mixture. The reaction was stirred at room temperature for 2 h. The mixture was diluted with water (20 mL) and extracted with EtOAc (20 mL×3). The combined organic layers were washed with brine, dried over Na₂SO₄, filtered and concentrated. The residue obtained was purified by RP-column (eluent: 62% ACN in water) to afford tert-butyl (S)-2-(((6-benzyl-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate (290 mg, 64%) as a yellow oil. LCMS m/z=697.4 [M+H]⁺;

Step 2: tert-butyl (S)-6-(4,4-difluorocyclohexyl)-3-fluoro-2-(((2-(3,3,3-trifluoro-2,2-dimethylpropanoyl)-2,6-diazaspiro[3.4]octan-8-yl)methoxy)methyl)benzoate To a solution of tert-butyl (S)-2-(((6-benzyl-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate (126 mg, 0.18 mmol) in EtOAc (1 mL) was added 10% Pd/C (50 mg). The mixture was stirred at room temperature under H₂ atmosphere overnight. The mixture was filtered through celite and concentrated, purified by prep-TLC (eluent: DCM:MeOH=10:1) to afford tert-butyl (S)-6-(4,4-difluorocyclohexyl)-3-fluoro-2-(((2-(3,3,3-trifluoro-2,2-dimethylpropanoyl)-2,6-diazaspiro[3.4]octan-8-yl)methoxy)methyl)benzoate (50 mg, 45%) as a colorless oil. LCMS m/z=607.4 [M+H]⁺; ¹H NMR (400 MHz, Chloroform-d) δ 7.30-7.27 (m, 1H), 7.09 (t, J=8.8 Hz, 1H), 4.52 (s, 2H), 3.63 (s, 1H), 3.50 (d, J=4.6 Hz, 3H), 3.25 (d, J=11.2 Hz, 2H), 3.00 (s, 2H), 2.61 (s, 2H), 2.42 (s, 1H), 2.22 (d, J=5.6 Hz, 3H), 2.06-1.69 (m, 6H), 1.60 (s, 9H), 1.36 (s, 6H).

Step 3: tert-butyl (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate To a solution of tert-butyl (S)-6-(4,4-difluorocyclohexyl)-3-fluoro-2-(((2-(3,3,3-trifluoro-2,2-dimethylpropanoyl)-2,6-diazaspiro[3.4]octan-8-yl)methoxy)methyl)benzoate (120 mg, 0.19 mmol) in toluene (2 mL) was added 7-bromo-4,5-difluorobenzo[d]thiazole (54 mg, 0.21 mmol), Cs₂CO₃ (96 mg, 0.29 mmol) and RuPhos Pd G3 (33 mg, 0.39 mmol). The mixture was stirred at 100° C. under N₂ atmosphere for 2 h. The mixture was filtered through celite and concentrated. The crude was purified by prep-TLC (eluent: DCM:MeOH=30:1) to afford tert-butyl (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate (100 mg, 65%) as a yellow oil. LCMS m/z=720.2 [M+H]⁺; ¹H NMR (400 MHz, Chloroform-d) δ 8.95 (s, 1H), 7.23 (d, J=8.6 Hz, 1H), 7.04 (t, J=8.6 Hz, 1H), 6.34 (dd, J=12.8, 5.8 Hz, 1H), 4.58 (dd, J=9.6, 1.6 Hz, 3H), 4.42-4.11 (m, 2H), 4.11-3.81 (m, 2H), 3.69 (d, J=17.6 Hz, 4H), 2.62 (d, J=6.8 Hz, 3H), 1.96-1.70 (m, 7H), 1.57 (s, 9H), 1.39 (s, 7H).

Step 4: (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoic acid I′-14 To a solution of tert-butyl (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoate (80 mg, 0.1 mmol) in DCM (1 mL) was added TFA (1 mL). The reaction mixture was stirred at room temperature for 3 h. The solvent was removed under vacuum. The residue was purified by prep-HPLC to afford (S)-2-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)-6-(4,4-difluorocyclohexyl)-3-fluorobenzoic acid (45 mg, 48%) as a yellow solid. LCMS m/z=720.3 [M+H]⁺; ¹H NMR (400 MHz, Methanol-d₄) δ 9.20 (s, 1H), 7.34 (t, J=6.8 Hz, 1H), 7.11 (t, J=9.0 Hz, 1H), 6.50 (dd, J=13.4, 5.8 Hz, 1H), 4.76-3.83 (m, 6H), 3.78-3.59 (m, 5H), 3.44 (dd, J=9.2, 5.8 Hz, 1H), 2.86-2.61 (m, 2H), 2.13 (s, 2H), 1.95-1.70 (m, 6H), 1.44-1.33 (m, 6H).

### (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(spiro[3.5]nonan-7-yl)benzoic acid I′-96

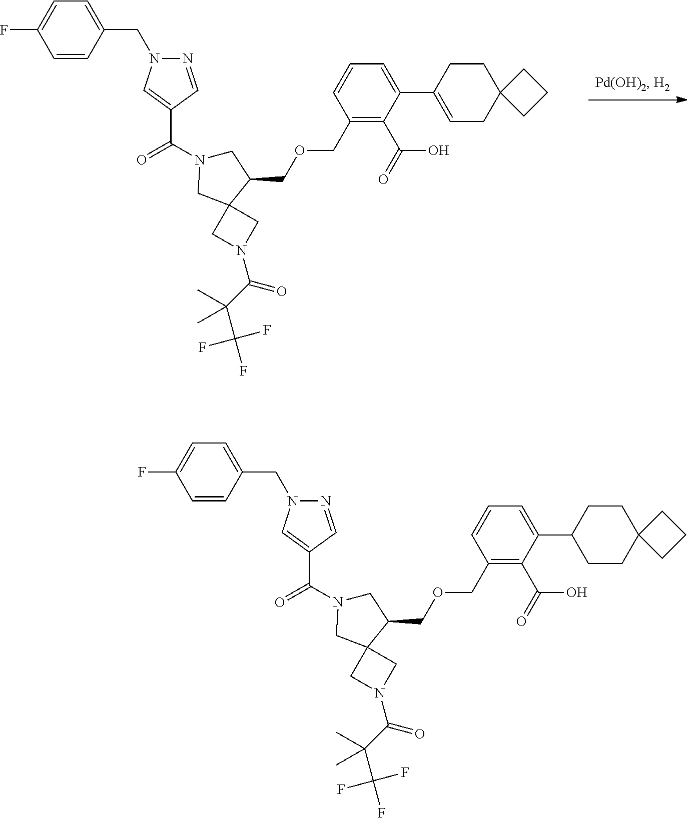 [SMILES: CC(C)(C(=O)N1CC2(CN(C(=O)c3cnn(Cc4ccc(F)cc4)c3)C[C@H]2COCc2cccc(C3=CCC4(CCC4)CC3)c2C(=O)O)C1)C(F)(F)F ; CC(C)(C(=O)N1CC2(CN(C(=O)c3cnn(Cc4ccc(F)cc4)c3)C[C@H]2COCc2cccc(C3CCC4(CCC4)CC3)c2C(=O)O)C1)C(F)(F)F]

To a solution of (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(spiro[3.5]non-6-en-7-yl)benzoic acid (40 mg, 0.05 mmol) in MeOH (3 mL) was added Pd(OH)₂ (16 mg), the reaction mixture was stirred under a H₂ atmosphere at 50° C for 3 h. The solvent was removed under vacuum and purified by prep-TLC (eluent: DCM:MeOH=20:1) to afford (S)-2-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)-6-(spiro[3.5]nonan-7-yl)benzoic acid (16.6 mg, 43%) as a white solid. LCMS m/z=739.5 [M+H]; ¹H NMR (400 MHz, DMSO-d₆) δ 8.37 (d, J=26.6 Hz, 1H), 7.82 (d, J=10.8 Hz, 1H), 7.33 (dd, J=8.4, 5.4 Hz, 2H), 7.16 (q, J=8.4, 7.6 Hz, 5H), 5.34 (s, 2H), 4.59-3.49 (m, 12H), 2.61-2.54 (m, 1H), 1.86-1.77 (m, 6H), 1.70-1.56 (m, 4H), 1.47-1.20 (m, 11H).

### (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid I′-97

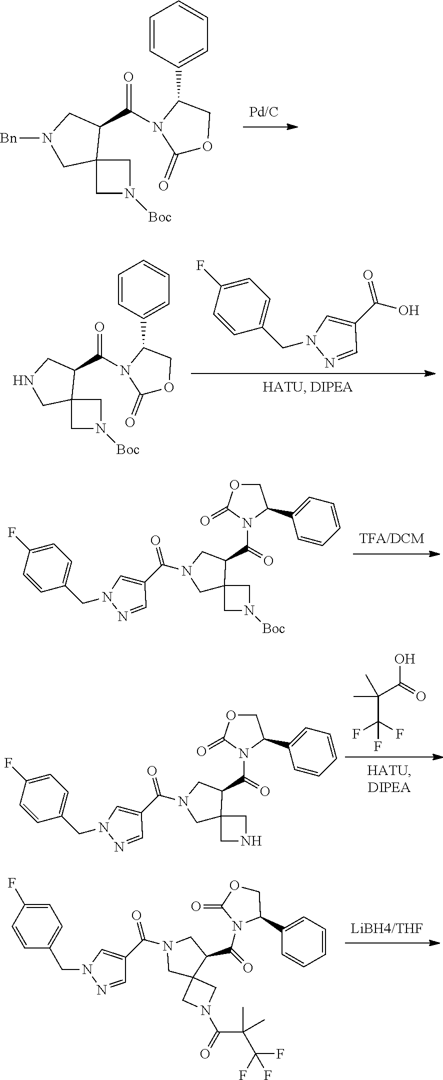

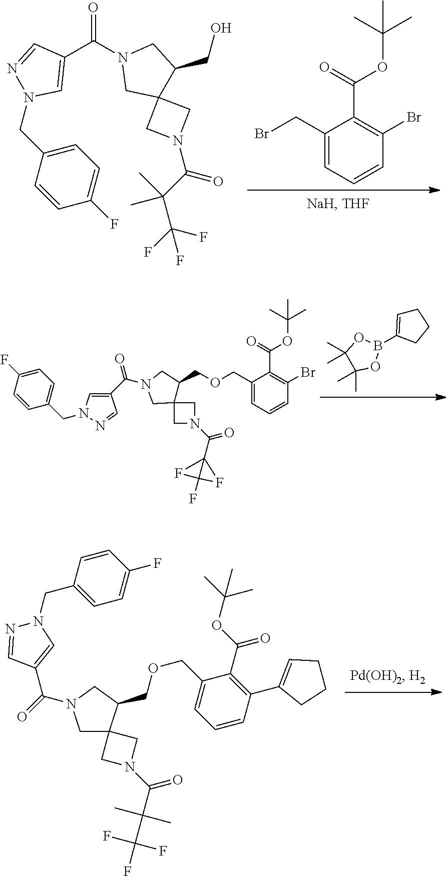

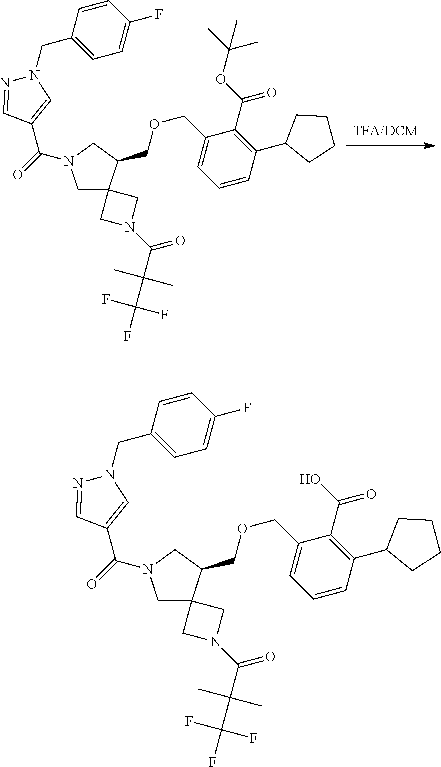 [SMILES: CC(C)(C)OC(=O)c1c(COC[C@@H]2CN(C(=O)c3cnn(Cc4ccc(F)cc4)c3)CC23CN(C(=O)C(C)(C)C(F)(F)F)C3)cccc1C1CCCC1 ; CC(C)(C(=O)N1CC2(CN(C(=O)c3cnn(Cc4ccc(F)cc4)c3)C[C@H]2COCc2cccc(C3CCCC3)c2C(=O)O)C1)C(F)(F)F]

Step 1: tert-butyl (S)-8-((R)-2-oxo-4-phenyloxazolidine-3-carbonyl)-2,6-diazaspio[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (1 g, 2 mmol) in EtOAc (8 mL) was added Pd/C (400 mg). The reaction mixture was stirred at 50° C. for 36 h with H₂ balloon. The mixture was filtrated with MeOH and concentrated under vacuum to afford tert-butyl (S)-8-((R)-2-oxo-4-phenyloxazolidine-3-carbonyl)-2,6-di azaspiro[3.4]octane-2-carb oxyl ate (0.7 g, 88%) as a yellow oil. LCMS w % z=402.2 [M+H]⁺.

Step 2: tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate: To a solution of 1-(4-fluorobenzyl)-1H-pyrazole-4-carboxylic acid (1.9 g, 8.7 mmol) and HATU (3.3 g, 8.7 mmol) in DCM (50 mL) was added DIPEA (3.4 g, 26.1 mmol). The mixture was stirred at room temperature for 30 min, tert-butyl (S)-8-((R)-2-oxo-4-phenyloxazolidine-3-carbonyl)-2,6-diazaspiro[3.4]octane-2-carboxylate (3.5 g, 8.7 mmol) was added and stirred at room temperature for another 2 h. The mixture was diluted with water (80 mL), extracted with DCM (100 mL×2) and the combined organic layers washed with brine, dried over Na₂SO₄ and filtered. The solvent was removed and the residue purified by column chromatography on silica gel (eluent: DCM:MeOH=50:1) to afford tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate (2.3 g, 43%) as a yellow oil. LCMS m/z=604.3 [M+H]⁺; ¹H NMR (400 MHz, DMSO-d₆) δ 8.35-8.27 (m, 1H), 7.81-7.74 (m, 1H), 7.39-7.05 (m, 9H), 5.47-5.27 (m, 3H), 4.80-4.70 (m, 1H), 4.34-3.57 (m, 10H), 1.38 (s, 9H).

Step 3: (R)-3-((S)-6-(1-(4-fluorobenzyl)-1H-pyrazole-4-carbonyl)-2,6-diazaspiro[3.4]octane-8-carbonyl)-4-phenyloxazolidin-2-one: To a solution of tert-butyl (S)-6-(1-(4-fluorobenzyl)-1H-pyrazole-4-carbonyl)-8-((R)-2-oxo-4-phenyloxazolidine-3-carbonyl)-2,6-diazaspiro[3.4]octane-2-carboxylate (2.3 g, 3.8 mmol) in DCM (4 mL) was added TFA (2 ml). The reaction mixture was stirred at room temperature for 2 h. The mixture was concentrated in vacuo to afford (R)-3-((S)-6-(1-(4-fluorobenzyl)-1H-pyrazole-4-carbonyl)-2,6-diazaspiro[3.4]octane-8-carbonyl)-4-phenyloxazolidin-2-one (1.9 g, 100%) as a yellow oil. LCMS m/z=504.2 [M+H]⁺.

Step 4: (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one: To a solution of 3,3,3-trifluoro-2,2-dimethylpropanoic acid (588 mg, 3.8 mmol) and HATU (1.4 g, 3.8 mmol) in DCM (20 mL) was added DIPEA (1.5 g, 15.2 mmol). The mixture was stirred at room temperature for 30 min. (R)-3-((S)-6-(1-(4-fluorobenzyl)-1H-pyrazole-4-carbonyl)-2,6-diazaspiro[3.4]octane-8-carbonyl)-4-phenyloxazolidin-2-one (1.9 g, 3.8 mmol) was added and stirred at room temperature for another 2 h. The mixture was diluted with water (80 mL), extracted with DCM (100 mL×2) and the combined organic layers washed with brine, dried over Na₂SO₄ and filtered. The solvent was removed and the residue purified by column chromatography on silica gel (eluent: DCM:MeOH=40:1) to afford (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one (1.2 g, 50%) as white solid. LCMS m/z=642.2 [M+H]⁺; ¹H NMR (400 MHz, Chloroform-d) δ 7.79-7.65 (m, 2H), 7.39-7.28 (m, 2H), 7.25-7.09 (m, 5H), 7.05 (t, J=8.4 Hz, 2H), 5.43-5.37 (m, 1H), 5.29-5.22 (m, 2H), 4.79-4.72 (m, 1H), 4.50-4.25 (m, 4H), 4.11-3.82 (m, 6H), 1.39 (s, 6H).

Step 5: (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one: To a solution of (R)-3-((S)-6-(1-(4-fluorobenzyl)-1H-pyrazole-4-carbonyl)-2-(3,3,3-trifluoro-2,2-dimethylpropanoyl)-2,6-diazaspiro[3.4]octane-8-carbonyl)-4-phenyloxazolidin-2-one (500 mg, 0.78 mmol) in THF (10 mL) at −78° C. was added Lithium borohydride (2 M/L in THF, 273 ul, 0.55 mmol). The reaction mixture was stirred at 0° C. for 4 h then diluted with water (10 mL), extracted with EtOAc (50 mL), the combined organic layers were washed with brine, dried over Na₂SO₄, filtered and concentrated in vacuo. The residue was purified by prep-TLC (eluent: DCM:MeOH=20:1) to afford (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (140 mg, 37%) as a white solid. LCMS m/z=483.2 [M+H]⁺; ¹H NMR (400 MHz, DMSO-d₆) δ 8.35-8.31 (m, 1H), 7.81 (d, J=10.4 Hz, 1H), 7.35-7.30 (m, 2H), 7.18 (t, J=8.8 Hz, 2H), 5.34 (s, 2H), 4.81-4.76 (m, 1H), 4.40-3.44 (m, 10H), 2.03-1.96 (m, 1H), 1.36-1.32 (m, 6H).

Step 6: To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (100 mg, 0.21 mmol) and tert-butyl 2-bromo-6-(bromomethyl)benzoate (74 mg, 0.21 mmol) in THF at 0° C. was added sodium hydride (42 mg, 1.1 mmol), Then the mixture was warmed to room temperature and stirred at RT for 3 h. The mixture was diluted with water (30 mL) and extracted with EtOAc (70 mL×2). The combined organic layers were dried over Na₂SO₄, filtered and concentrated. The mixture was purified by prep-TLC (eluent: DCM:MeOH=30:1) to afford tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (140 mg, 70% yield) as a white solid. LCMS m/z=751.2 [M+H]⁺; ¹H NMR (400 MHz, Chloroform-d) δ 8.01-7.73 (m, 2H), 7.56-7.45 (m, 1H), 7.25-7.14 (m, 4H), 7.08-6.97 (m, 2H), 5.27 (s, 2H), 4.67-3.68 (m, 9H), 3.63-3.39 (m, 3H), 2.58-2.46 (m, 1H), 1.58 (s, 9H), 1.37 (s, 6H).

Step 7: tert-butyl (S)-2-(cyclopent-1-en-1-yl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate: To a solution of tert-butyl (S)-2-bromo-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (100 mg, 0.13 mmol) in dioxane (3 mL) and H₂O (1 mL) was added 2-(cyclopent-1-en-1-yl)-4,4,5,5-tetramethyl-1,3,2-dioxaborolane (32 mg, 0.16 mmol), Pd(PPh₃)₄ (9 mg, 0.008 mmol) and K₃PO₄ (56 mg, 0.27 mmol). The reaction mixture was stirred at 100° C. for 3 h. The mixture was diluted with water (20 mL) and extracted with EtOAc (30 mL×3). The combined organic layers were washed with brine, dried over Na₂SO₄, filtered and concentrated. The residue obtained was purified by prep-TLC (eluent: DCM:MeOH=20:1) to afford tert-butyl (S)-2-(cyclopent-1-en-1-yl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (95 mg, 97%) as a colorless oil. LCMS m/z=683.4 [M+H−56]⁺; ¹H NMR (400 MHz, DMSO-d₆) δ 8.32 (d, J=6.2 Hz, 1H), 7.81 (d, J=13.8 Hz, 1H), 7.64-7.54 (m, 1H), 7.34-7.16 (m, 6H), 5.70 (s, 1H), 5.34 (s, 2H), 4.60-3.34 (m, 13H), 2.64-2.58 (m, 2H), 2.47-2.40 (m, 2H), 1.99-1.91 (m, 2H), 1.45 (d, J=11.4 Hz, 9H), 1.33-1.27 (m, 6H).

Step 8: tert-butyl (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate To a solution of tert-butyl (S)-2-(cyclopent-1-en-1-yl)-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (90 mg, 0.12 mmol) in MeOH (3 mL) was added Pd(OH)₂ (36 mg), the reaction mixture was stirred under a H₂ atmosphere at 50° C. for 5 h. The mixture was filtered and concentrated to afford tert-butyl (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (80 mg, 88%) which was used directly in the next step. LCMS m/z=685.3 [M+H−56]⁺; ¹H NMR (400 MHz, DMSO-d₆) δ 8.33 (d, J=5.6 Hz, 1H), 7.81 (d, J=11.6 Hz, 1H), 7.35-7.28 (m, 4H), 7.21-7.14 (m, 3H), 5.34 (s, 2H), 4.57-4.13 (m, 4H), 3.97-3.37 (m, 9H), 2.99 (q, J=8.8 Hz, 1H), 1.98-1.90 (m, 2H), 1.81-1.74 (m, 2H), 1.67-1.53 (m, 4H), 1.50 (d, J=13.6 Hz, 9H), 1.32-1.26 (s, 6H).

Step 9: (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid To a solution of tert-butyl (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoate (80 mg, 0.11 mmol) in DCM (3 mL) was added TFA (1.5 mL). The reaction mixture was stirred at room temperature for 3 h. The solvent was removed under vacuum and purified by prep-TLC (eluent: DCM:MeOH=20:1) to afford (S)-2-cyclopentyl-6-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)benzoic acid (30 mg, 73%) as a white solid. LCMS m/z=685.4 [M+H]⁺; ¹H NMR (400 MHz, DMSO-d₆) δ 8.38 (m, 1H), 7.82 (d, J=9.6 Hz, 1H), 7.36-7.30 (m, 2H), 7.27-7.09 (m, 5H), 5.34 (s, 2H), 4.61-3.37 (m, 13H), 3.13-3.03 (m, 1H), 1.98-1.89 (m, 2H), 1.80-1.71 (m, 2H), 1.63-1.45 (m, 4H), 1.31 (s, 6H).

### (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-98

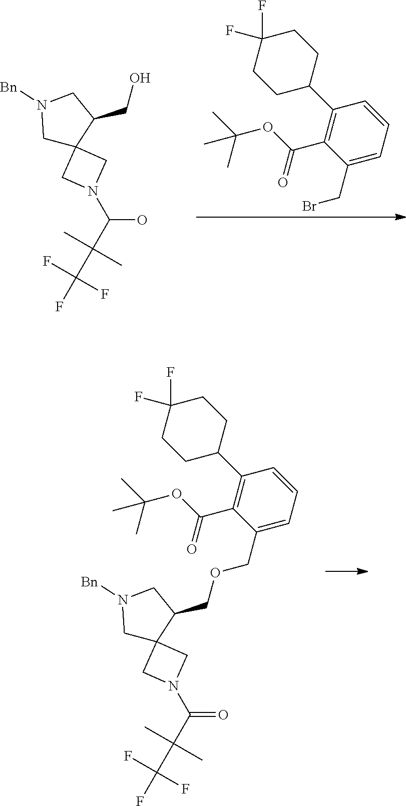 [SMILES: CC(C)(C(O)N1CC2(CN(Cc3ccccc3)C[C@H]2CO)C1)C(F)(F)F ; CC(C)(C)OC(=O)c1c(CBr)cccc1C1CCC(F)(F)CC1 ; CC(C)(C)OC(=O)c1c(COC[C@@H]2CN(Cc3ccccc3)CC23CN(C(=O)C(C)(C)C(F)(F)F)C3)cccc1C1CCC(F)(F)CC1]

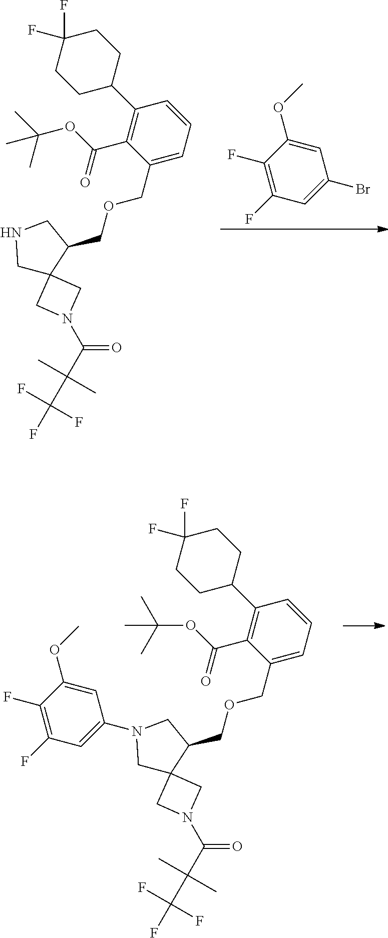 [SMILES: CC(C)(C)OC(=O)c1c(COC[C@@H]2CNCC23CN(C(=O)C(C)(C)C(F)(F)F)C3)cccc1C1CCC(F)(F)CC1 ; COc1cc(Br)cc(F)c1F ; COc1cc(N2C[C@@H](COCc3cccc(C4CCC(F)(F)CC4)c3C(=O)OC(C)(C)C)C3(CN(C(=O)C(C)(C)C(F)(F)F)C3)C2)cc(F)c1F]

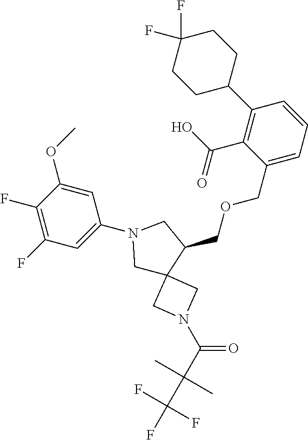 [SMILES: COc1cc(N2C[C@@H](COCc3cccc(C4CCC(F)(F)CC4)c3C(=O)O)C3(CN(C(=O)C(C)(C)C(F)(F)F)C3)C2)cc(F)c1F]

Step 1: tert-butyl (S)-2-(((6-benzyl-2-(3,3,3-trifluoro-2,2-dimethylpropanoyl)-2,6-diazaspiro[3.4]octan-8-yl)methoxy)methyl)-6-(4,4-difluorocyclohexyl)benzoate: To a solution of (S)-1-(6-benzyl-8-(hydroxymethyl)-2,6-diazaspiro[3.4]octan-2-yl)-3,3,3-trifluoro-2,2-dimethylpropan-1-one (215 mg, 0.58 mmol), tert-butyl 2-(bromomethyl)-6-(4,4-difluorocyclohexyl)benzoate (271 mg, 0.7 mmol) in THF (10 mL) at 0° C. was added sodium hydride (116 mg, 2.9 mmol), Then the mixture was warmed to room temperature and stirred at room temperature for 2 h. The mixture was diluted with water (20 mL) and extracted with EtOAc (20 mL×3). The combined organic layers were dried over Na₂SO₄, filtered and concentrated. The mixture was purified by prep-TLC (DCM/MeOH=15/1) to afford tert-butyl (S)-2-(((6-benzyl-2-(3,3,3-trifluoro-2,2-dimethylpropanoyl)-2,6-diazaspiro[3.4]octan-8-yl)methoxy)methyl)-6-(4,4-difluorocyclohexyl)benzoate (269 mg, 68% yield) as a white solid. LCMS m/z=679.5 [M+H]⁺; ¹H NMR (400 MHz, Methanol-d₄) δ 7.39-7.22 (m, 8H), 4.62-4.48 (m, 2H), 4.40-4.28 (m, 1H), 4.19-4.06 (m, 1H), 3.94-3.82 (m, 1H), 3.66-3.59 (m, 2H), 3.54-3.47 (m, 2H), 2.96-2.84 (m, 2H), 2.83-2.73 (m, 1H), 2.64 (d, J=9.6 Hz, 1H), 2.43 (s, 1H), 2.28-2.13 (m, 3H), 2.06-1.99 (m, 1H), 1.94-1.76 (m, 6H), 1.62 (s, 9H), 1.30 (s, 6H).

Step 2: tert-butyl (S)-2-(4,4-difluorocyclohexyl)-6-(((2-(3,3,3-trifluoro-2,2-dimethylpropanoyl)-2,6-diazaspiro[3.4]octan-8-yl)methoxy)methyl)benzoate: To a solution of ethyl 6-benzyl-2-((S)-2,2-dimethylcyclopropane-1-carbonyl)-2,6-diazaspiro[3.4]octane-8-carboxylate (269 mg, 0.4 mmol) in EtOAc (4 mL) was added 40% Pd/C (108 mg). The reaction mixture was stirred under a H₂ atmosphere for 24 h. The mixture was filtered through celite and concentrated to afford tert-butyl (S)-2-(4,4-difluorocyclohexyl)-6-(((2-(3,3,3-trifluoro-2,2-dimethylpropanoyl)-2,6-diazaspiro[3.4]octan-8-yl)methoxy)methyl)benzoate (200 mg, 86%) which was used directly in the next step. LCMS m/z=589.3 [M+H]⁺.¹H NMR (400 MHz, Chloroform-d) δ 7.34 (t, J=7.6 Hz, 1H), 7.23 (d, J=8.0 Hz, 2H), 4.51 (s, 2H), 4.37-4.18 (m, 1H), 4.12-4.03 (m, 1H), 4.01-3.85 (m, 1H), 3.81-3.66 (m, 1H), 3.52-3.45 (m, 2H), 3.24-3.14 (m, 2H), 3.12-3.04 (m, 1H), 2.76-2.68 (m, 2H), 2.34-2.28 (m, 1H), 2.25-2.19 (m, 2H), 2.03-1.99 (m, 1H), 1.98-1.92 (m, 2H), 1.84-1.78 (m, 3H), 1.60 (s, 9H), 1.36 (s, 6H).

Step 3: tert-butyl (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate: To a solution of tert-butyl (S)-2-(4,4-difluorocyclohexyl)-6-(((2-(3,3,3-trifluoro-2,2-dimethylpropanoyl)-2,6-diazaspiro[3.4]octan-8-yl)methoxy)methyl)benzoate (170 mg, 0.31 mmol), 5-bromo-1,2-difluoro-3-methoxybenzene (71 mg, 0.32 mmol) and Cs₂CO₃ (141 mg, 0.43 mmol) in toluene (5 mL) was added RuPhosPdG3 (48 mg, 0.06 mmol). The mixture was stirred at 100° C. for 2 h. The mixture was diluted with water (20 mL) and extracted with EtOAc (20 mL×3). The combined organic layers were dried over Na₂SO₄, filtered and concentrated. The mixture was purified by prep-TLC (DCM/MeOH=50/1) to afford tert-butyl (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (185 mg, 88% yield) as a yellow solid. LCMS m/z=731.5 [M+H]⁺; ¹H NMR (400 MHz, Chloroform-d) δ 7.33 (t, J=7.6 Hz, 1H), 7.25-7.18 (m, 2H), 5.91-5.82 (m, 2H), 4.54 (s, 2H), 4.46-3.91 (m, 4H), 3.88 (s, 3H), 3.54-3.34 (m, 4H), 3.14 (dd, J=9.6, 5.6 Hz, 1H), 2.79-2.70 (m, 1H), 2.63-2.54 (m, 1H), 2.27-2.18 (m, 2H), 2.05-1.90 (m, 3H), 1.85-1.73 (m, 4H), 1.59 (s, 9H), 1.37 (s, 6H).

Step 4: (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid: To a solution of tert-butyl (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoate (100 mg, 0.14 mmol) in DCM (2 mL) was added TFA (1 mL). The reaction mixture was stirred at room temperature for 2 h. The mixture was purified by prep-HPLC to afford (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid (40 mg, 43%) as a white solid. LCMS m/z=675.4 [M+H]⁺; ¹H NMR (400 MHz, DMSO-d₆) δ 7.36-7.22 (m, 3H), 6.04 (d, J=6.6 Hz, 2H), 4.61-3.92 (m, 5H), 3.84 (s, 3H), 3.75-3.58 (m, 2H), 3.54-3.44 (m, 3H), 3.41-3.36 (m, 1H), 3.11 (dd, J=9.8, 5.8 Hz, 1H), 2.83-2.72 (m, 1H), 2.65-2.57 (m, 1H), 2.16-2.05 (m, 2H), 1.93-1.63 (m, 6H), 1.31 (s, 6H).

### (S)-2-(((6-(3,4-difluoro-5-methoxyphenyl)-2-(3,3,3-trifluoro-2,2-dimethylpropanoyl)-2,6-diazaspiro[3.4]octan-8-yl)methoxy)methyl)-6-(4,4-difluorocyclohexyl)benzoic acid I′-99

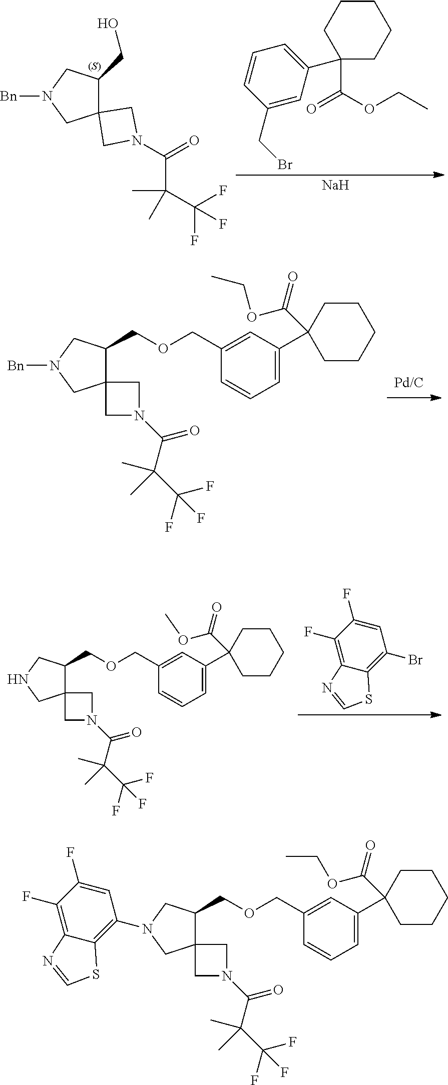

Step 1: ethyl (S)-1-(3-(((6-benzyl-2-(3,3,3-trifluoro-2,2-dimethylpropanoyl)-2,6-diazaspiro[3.4]octan-8-yl)methoxy)methyl)phenyl)cyclohexane-1-carboxylate To a solution of (S)-1-(6-benzyl-8-(hydroxymethyl)-2,6-diazaspiro[3.4]octan-2-yl)-3,3,3-trifluoro-2,2-dimethylpropan-1-one (150 mg, 0.41 mmol) and ethyl 1-(3-(bromomethyl)phenyl)cyclohexane-1-carboxylate (180 mg, 0.56 mmol) was added NaH (81 mg, 2.03 mmol). The mixture was stirred at room temperature under a N₂ atmosphere overnight. The mixture was diluted with water (50 mL) and extracted with EtOAc (50 mL×3). The combined organic layers were washed with brine, dried over Na₂SO₄, filtered and concentrated. The residue was purified by prep-TLC (eluent: DCM:MeOH=20:1) to afford ethyl (S)-1-(3-(((6-benzyl-2-(3,3,3-trifluoro-2,2-dimethylpropanoyl)-2,6-diazaspiro[3.4]octan-8-yl)methoxy)methyl)phenyl)cyclohexane-1-carboxylate. LCMS m/z=615.5 [M+H]⁺.

Step 2: ethyl (S)-1-(3-(((2-(3,3,3-trifluoro-2,2-dimethylpropanoyl)-2,6-diazaspiro[3.4]octan-8-yl)methoxy)methyl)phenyl)cyclohexane-1-carboxylate To a solution of ethyl (S)-1-(3-(((6-benzyl-2-(3,3,3-trifluoro-2,2-dimethylpropanoyl)-2,6-diazaspiro[3.4]octan-8-yl)methoxy)methyl)phenyl)cyclohexane-1-carboxylate (120 mg, 0.195 mmol) in EtOAc (3 mL) was added 10% Pd/C (48 mg). The reaction mixture was stirred at 40° C. under a H₂ atmosphere for 48 h. The mixture was filtered and concentrated to afford ethyl (S)-1-(3-(((2-(3,3,3-trifluoro-2,2-dimethylpropanoyl)-2,6-diazaspiro[3.4]octan-8-yl)methoxy)methyl)phenyl)cyclohexane-1-carboxylate (70 mg, 68%) which was used directly in the next step. LCMS m/z=525.3[M+H]⁺.

Step 3: ethyl (S)-1-(3-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)phenyl)cyclohexane-1-carboxylate To a solution of ethyl (S)-1-(3-(((2-(3,3,3-trifluoro-2,2-dimethylpropanoyl)-2,6-diazaspiro[3.4]octan-8-yl)methoxy)methyl)phenyl)cyclohexane-1-carboxylate (50 mg, 0.095 mmol) in toluene (2 mL) was added 7-bromo-4,5-difluorobenzo[d]thiazole (28 mg, 0.115 mmol), Cs₂CO₃ (19 mg, 0.057 mmol) and RuPhos Pd G3 (6 mg, 0.008 mmol). The resulting mixture was stirred under N₂ atmosphere at 100° C. for 2 h. The mixture was diluted with water (30 mL), extracted with EtOAc (50 mL×3). The combined organic layers were washed with brine, dried over Na₂SO₄, filtered and concentrated. The residue was purified by prep-TLC (eluent: DCM:MeOH=40:1) to afford ethyl (S)-1-(3-(((6-(4,5-difluorobenzo[d]thiazol-7-yl)-2-(3,3,3-trifluoro-2,2-dimethylpropanoyl)-2,6-diazaspiro[3.4]octan-8-yl)methoxy)methyl)phenyl)cyclohexane-1-carboxylate (4 mg, 6%). LCMS m/z=716.3 [M+Na]*; ¹H NMR (400 MHz, Methanol-d₄) δ 9.21 (s, 1H), 7.35 (d, J=1.8 Hz, 1H), 7.31-7.23 (m, 2H), 7.20-7.14 (m, 1H), 6.53 (dd, J=13.4, 5.8 Hz, 1H), 4.53 (s, 4H), 4.08 (t, 1=7.2 Hz, 2H), 4.03-3.83 (m, 1H), 3.82-3.64 (m, 5H), 3.48 (d, J=9.6 Hz, 1H), 2.73 (q, J=6.6 Hz, 1H), 2.43 (d, J=12.6 Hz, 2H), 1.70-1.59 (m, 5H), 1.51-1.34 (m, 9H), 1.25 (d, J=3.4 Hz, 1H), 1.13 (t, J=7.2 Hz, 3H).

### Ethyl (S)-1-(3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)phenyl)cyclopentane-1-carboxylate I′-100

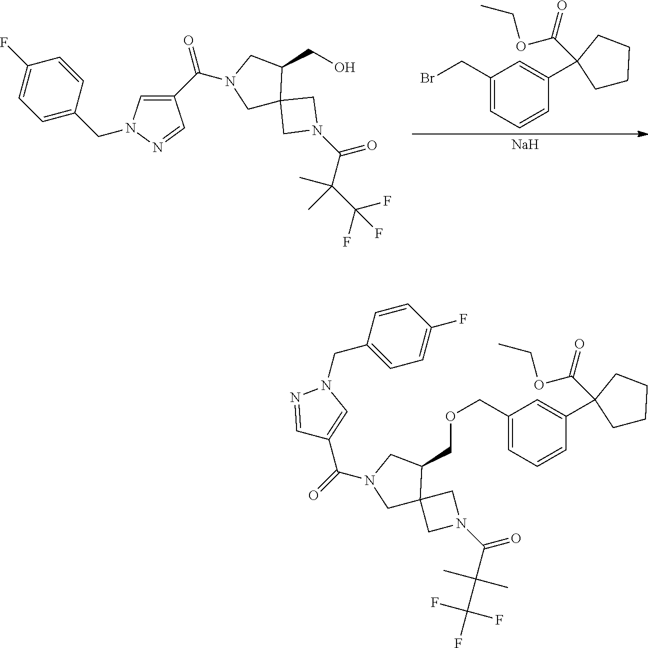

To a solution of (S)-3,3,3-trifluoro-1-(6-(1-(4-fluorobenzyl)-1H-pyrazole-4-carbonyl)-8-(hydroxymethyl)-2,6-diazaspiro[3.4]octan-2-yl)-2,2-dimethylpropan-1-one (100 mg, 0.2 mmol) in dry THF (1 mL) at 0° C. were added ethyl 1-(3-(bromomethyl)phenyl)cyclopentane-1-carboxylate (80 mg, 0.24 mmol) and NaH (20 mg, 0.5 mmol). The reaction mixture was stirred at room temperature overnight then diluted with water (10 mL) and extracted with EtOAc (25 mL×3). The combined organic layers were washed with brine, dried over Na₂SO₄, filtered and concentrated in vacuo. The residue was purified by prep-TLC (eluent: DCM:MeOH=15:1) to afford the ethyl (S)-1-(3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)phenyl)cyclopentane-1-carboxylate (66 mg, 44%) as a white solid. LCMS m/z=713.2 [M+H]⁺; ¹H NMR (400 MHz, DMSO-d₆) δ 8.34 (d, J=4.4 Hz, 1H), 7.81 (d, J=13.2 Hz, 1H), 7.34-7.13 (m, 8H), 5.34 (s, 2H), 4.48 (s, 2H), 4.42-3.50 (m, 12H), 2.70-2.51 (m, 3H), 1.84-1.56 (m, 6H), 1.30 (s, 6H), 1.05 (q, J=6.6 Hz, 3H).

### (S)-1-(3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)phenyl)cyclopentane-1-carboxylic acid I′-101

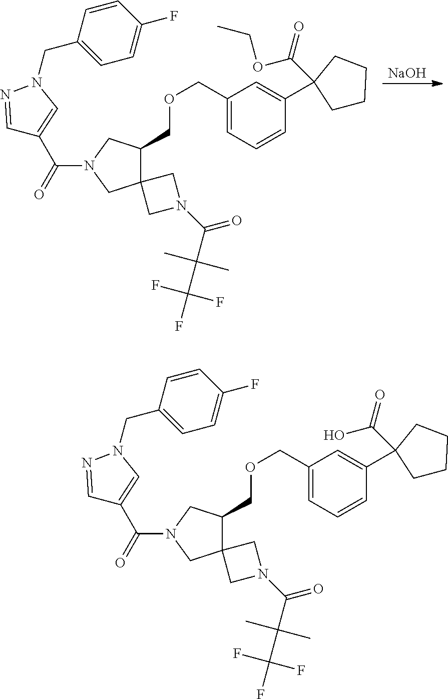

To a solution of ethyl (S)-1-(3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)phenyl)cyclopentane-1-carboxylate (66 mg, 0.09 mmol) in a mixture of THF/EtOH/H₂O (2 mL/0.5 mL/0.5 mL) at 0° C. was added a solution of NaOH (18 mg, 0.45 mmol). The reaction mixture was stirred at 50° C. overnight then diluted with water (10 mL) and extracted with EtOAc (20 mL×3). The combined organic layers were washed with brine, dried over Na₂SO₄, filtered and concentrated in vacuo. The residue was purified by prep-TLC (eluent: DCM:MeOH=15:1) to afford (S)-1-(3-(((6-(1-(4-fluorobenzyl)-1H-pyrazole-4-carbonyl)-2-(3,3,3-trifluoro-2,2-dimethylpropanoyl)-2,6-diazaspiro[3.4]octan-8-yl)methoxy)methyl)phenyl)cyclopentane-1-carboxylic acid (1.3 mg) as a white solid. LCMS m z=683.2 [M−H]⁻; ¹H NMR (400 MHz, CD₃OD) δ 8.20 (d, J=2.2 Hz, 1H), 7.90 (d, J=4.0 Hz, 1H), 7.38-7.04 (m, 8H), 5.36 (s, 2H), 4.51 (d, J=5.4 Hz, 2H), 4.44-3.48 (in, 10H), 2.71-2.55 (m, 3H), 2.22-1.71 (m, 6H), 1.39-1.33 (m, 6H).

### (R)-1-(3-((((S)-6-benzyl-2-(3,3,3-trifluoro-2,2-dimethylpropanoyl)-2,6-diazaspiro[3.4]octan-8-yl)methoxy)methyl)phenyl)piperidine-2-carboxylic acid I′-102

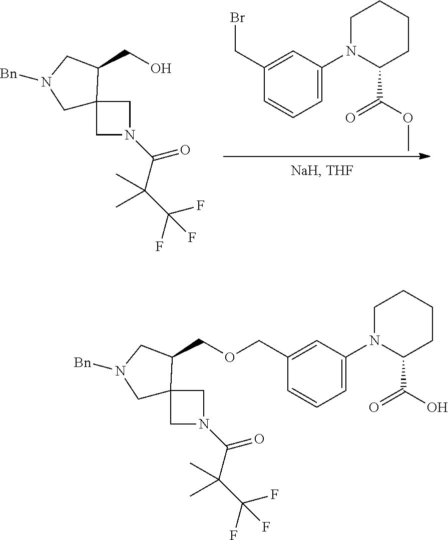 [SMILES: CC(C)(C(=O)N1CC2(CN(Cc3ccccc3)C[C@H]2CO)C1)C(F)(F)F ; COC(=O)[C@H]1CCCCN1c1cccc(CBr)c1 ; CC(C)(C(=O)N1CC2(CN(Cc3ccccc3)C[C@H]2COCc2cccc(N3CCCC[C@@H]3C(=O)O)c2)C1)C(F)(F)F]

To a solution of methyl-(R)-1-(3-(bromomethyl)phenyl)piperidine-2-carboxylate (100 mg, 0.32 mmol) in anhydrous THF (2 mL) at 0° C. was added NaH (38 mg, 1.6 mmol) over 10 min, then a solution of (S)-1-(6-benzyl-8-(hydroxymethyl)-2,6-diazaspiro[3.4]octan-2-yl)-3,3,3-trifluoro-2,2-dimethylpropan-1-one (118 mg, 0.32 mmol) in THE (1 mL) was added. The reaction was stirred at 70° C. overnight. The reaction was quenched by water (15 mL) and extracted with EtOAc (20 mL×3). The combined organic layers were dried over Na₂SO₄, filtered and concentrated. The residue was purified by prep-TLC (eluent: DCM/MeOH=20:1) to afford (R)-1-(3-((((S)-6-benzyl-2-(3,3,3-trifluoro-2,2-dimethylpropanoyl)-2,6-diazaspiro[3.4]octan-8-yl)methoxy)methyl)phenyl)piperidine-2-carboxylic acid (4.3 mg, 2%) as a yellow solid. LCMS m/z=588.4[M+H]⁺; ¹H NMR (400 MHz, DMSO-d₆) δ 7.35-7.20 (m, 5H), 7.10 (t, J=7.7 Hz, 1H), 6.81-6.72 (m, 2H), 6.61 (d, J=7.4 Hz, 1H), 4.50 (s, 1H), 4.37 (s, 2H), 4.25 (d, J=31.7 Hz, 1H), 4.04 (s, 1H), 3.76 (d, J=31.7 Hz, 2H), 3.55 (d, J=14.1 Hz, 3H), 3.46 (s, 3H), 3.19 (s, 2H), 2.87-2.77 (m, 2H), 2.41-2.32 (m, 1H), 2.18-2.09 (m, 2H), 1.81-1.41 (m, 4H), 1.29 (s, 6H).

TABLE 25
The compounds listed in Table 25 were synthesized from (S)-1-(6-benzyl-
8-(hydroxymethyl)-2,6-diazaspiro[3.4]octan-2-yl)-3,3,3-trifluoro-2,2-dimethylpropan-1-one
according to the procedures outlined for I′-102 using the appropriate commercially available
reagents and/or intermediates described elsewhere.

| Cmpd. | | | |
|---|---|---|---|
| No. | Compound | ¹HNMR | LCMS |
| I′-103 | 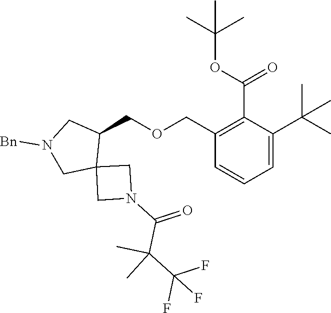 [SMILES: CC(C)(C)OC(=O)c1c(COC[C@@H]2CN(Cc3ccccc3)CC23CN(C(=O)C(C)(C)C(F)(F)F)C3)cccc1C(C)(C)C] | ¹H NMR (400 MHz, DMSO-d6) δ 7.62 (s, 1H), 7.49 − 7.26 (m, 7H), 4.45 (s, 2H), 4.38 − 3.45 (m, 10H), 3.22 − 2.93 (m, 2H), 2.67 (s, 1H), 1.56 (s, 9H), 1.36 (s, 9H), 1.28 (s, 6H). | m/z = 617.5 [M + H]⁺ |

### 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid I-130

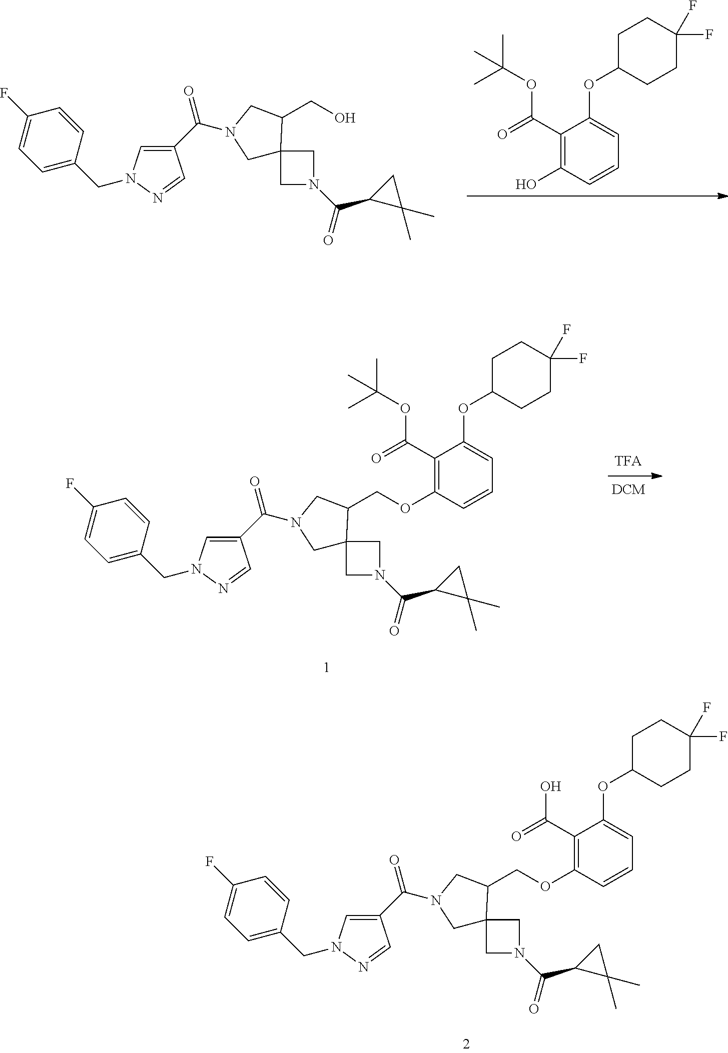 [SMILES: CC1(C)C[C@@H]1C(=O)N1CC2(CN(C(=O)c3cnn(Cc4ccc(F)cc4)c3)CC2CO)C1 ; CC(C)(C)OC(=O)c1c(O)cccc1OC1CCC(F)(F)CC1 ; CC(C)(C)OC(=O)c1c(OCC2CN(C(=O)c3cnn(Cc4ccc(F)cc4)c3)CC23CN(C(=O)[C@H]2CC2(C)C)C3)cccc1OC1CCC(F)(F)CC1 ; CC1(C)C[C@@H]1C(=O)N1CC2(CN(C(=O)c3cnn(Cc4ccc(F)cc4)c3)CC2COc2cccc(OC3CCC(F)(F)CC3)c2C(=O)O)C1]

Step 1: tert-butyl 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate To a solution of tert-butyl 2-((4,4-difluorocyclohexyl)oxy)-6-hydroxybenzoate (100 mg, 0.22 mmol), (2-((S)-2,2-dimethylcyclopropane-1-carbonyl)-8-(hydroxymethyl)-2,6-diazaspiro[3.4]octan-6-yl)(1-(4-fluorobenzyl)-1H-pyrazol-4-yl)methanone (89 mg, 0.27 mmol), DIAD (91 mg, 0.45 mmol) and PPh₃ (119 mg, 0.45 mmol) in toluene (1 mL) at 130° C. for 1 h under M.W. The mixture was concentrated and purified by prep-HPLC to afford tert-butyl 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate (30 mg, 17%) as a white solid. LCMS m/z=695.3 [M-99]⁺; ¹HNMR (400 MHz, Methanol-d₄) δ 8.22 (d, J=4.6 Hz, 1H), 7.92 (d, J=8.0 Hz, 1H), 7.41-7.23 (m, 3H), 7.13-7.03 (m, 2H), 6.81-6.65 (m, 2H), 5.36 (s, 2H), 4.68 (s, 1H), 4.42-4.22 (m, 2H), 4.22-3.91 (m, 5H), 3.84 (d, J=10.7 Hz, 2H), 2.85 (s, 1H), 2.23-1.94 (m, 5H), 1.90 (d, J=11.6 Hz, 4H), 1.63-1.41 (m, 10H), 1.30 (d, J=6.2 Hz, 2H), 1.17 (d, J=8.4 Hz, 2H), 1.05 (s, 4H), 0.75 (d, J=6.2 Hz, 1H).

Step 2: 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid To a solution of tert-butyl 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoate (60 mg, 0.22 mmol) in DCM (2 mL), TFA (1 mL) was added, the mixture was stirred at room temperature for 2 h. The mixture was concentrated and purified by prep-TLC (DCM:MeOH=20:1) to afford 2-((4,4-difluorocyclohexyl)oxy)-6-((2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)benzoic acid (40 mg, 62.5%) as a white solid. LCMS m/z=695.3 [M+H]⁺; ¹H NMR (400 MHz, Methanol-d₄) δ 8.26 (d, J=10.2 Hz, 1H), 7.95 (s, 1H), 7.33 (s, 3H), 7.07 (s, 2H), 6.73 (d, J=6.0 Hz, 2H), 5.36 (d, J=6.8 Hz, 2H), 4.56-4.42 (m, 2H), 4.36-3.82 (m, 9H), 2.96-2.77 (m, 1H), 2.16 (d, J=13.8 Hz, 2H), 2.07-1.97 (m, 2H), 1.87 (s, 4H), 1.64-1.37 (m, 1H), 1.22-0.97 (m, 7H), 0.76 (s, 1H).

TABLE 26
The compounds listed in Table 26 were synthesized from (2-((S)-2,2-
dimethylcyclopropane-1-carbonyl)-8-(hydroxymethyl)-2,6-diazaspiro[3.4]octan-6-yl)(1-(4-
fluorobenzyl)-1H-pyrazol-4-yl)methanone according to the procedures outlined for I-130 using
the appropriate commercially available reagents and/or intermediates described elsewhere.

| Cmpd. | | | |
|---|---|---|---|
| No. | Compound | ¹HNMR | LCMS |
| I-129 | 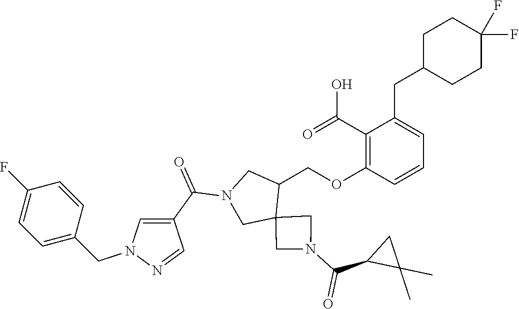 [SMILES: CC1(C)C[C@@H]1C(=O)N1CC2(CN(C(=O)c3cnn(Cc4ccc(F)cc4)c3)CC2COc2cccc(CC3CCC(F)(F)CC3)c2C(=O)O)C1] | ¹H NMR (400 MHz, CD₃OD) δ 8.24 (d, J = 9.4 Hz, 1H), 7.94 (d, J = 9.6 Hz, 1H), 7.37 − 7.27 (m, 3H), 7.11 − 7.04 (m, 2H), 7.00 − 6.93 (m, 1H), 6.90 − 6.85 (m, 1H), 5.36 (d, J = 6.2 Hz, 2H), 4.58 − 3.62 (m, 10H), 2.93 − 2.78 (m, 1H), 2.60 − 2.55 (m, 2H), 2.03 − 1.93 (m, 2H), 1.77 − 1.64 (m, 4H), 1.50 − 1.39 (m, 1H), 1.33 − 0.99 (m, 10H), 0.79 − 0.70 (m, 1H). | m/z = 693.5 [M + H]⁺ |

### 2-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzamide I′-104

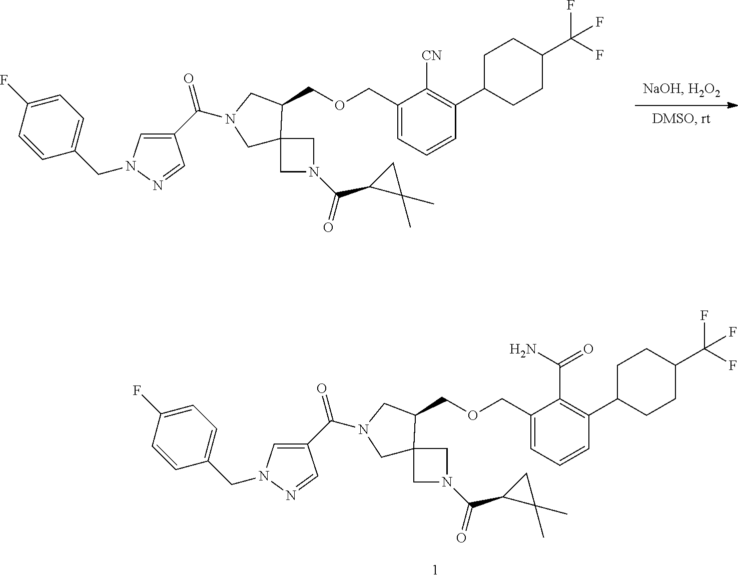 [SMILES: CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2C#N)C1 ; CC1(C)C[C@@H]1C(=O)N1CC2(CN(C(=O)c3cnn(Cc4ccc(F)cc4)c3)C[C@H]2COCc2cccc(C3CCC(C(F)(F)F)CC3)c2C(N)=O)C1]

To a solution of 2-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzonitrile (see i.e., I′-43) (100 mg, 0.14 mmol) in DMSO (5.0 mL) was added NaOH (0.4 g, 10 mmol) and H₂O₂ (30%, 1.0 mL), the mixture was stirred at room temperature for 14 h. The mixture was diluted with water (30 mL) and extracted with EtOAc (20 mL×2). The combined organic layers were washed with brine, dried over Na₂SO₄, filtered and concentrated. The residue obtained was purified by Prep-HPLC to afford 2-((((S)-2-((S)-2,2-dimethylcyclopropane-1-carbonyl)-6-(1-(4-fluorobenzyl)-1H-pyrazole-4-carbonyl)-2,6-diazaspiro[3.4]octan-8-yl)methoxy)methyl)-6-(4-(trifluoromethyl)cyclohexyl)benzamide (45 mg, 44%) as a white solid. LCMS m/z=723.3 [M+H]⁺; ¹H NMR (400 MHz, Methanol-d₄) δ 8.29-8.17 (m, 1H), 7.90 (s, 1H), 7.37-7.20 (m, 5H), 7.12-7.02 (m, 2H), 5.35 (s, 2H), 4.64-4.53 (m, 2H), 4.47-3.53 (m, 10H), 2.88-2.56 (m, 2H), 2.50-1.94 (m, 4H), 1.81-1.67 (m, 4H), 1.66-1.33 (m, 2H), 1.19-1.07 (m, 6H), 1.07-0.98 (m, 1H), 0.81-0.68 (m, 1H).

### Building Blocks

### 1-(4-chloro-2-fluorobenzyl)-1H-pyrazole-4-carboxylic acid

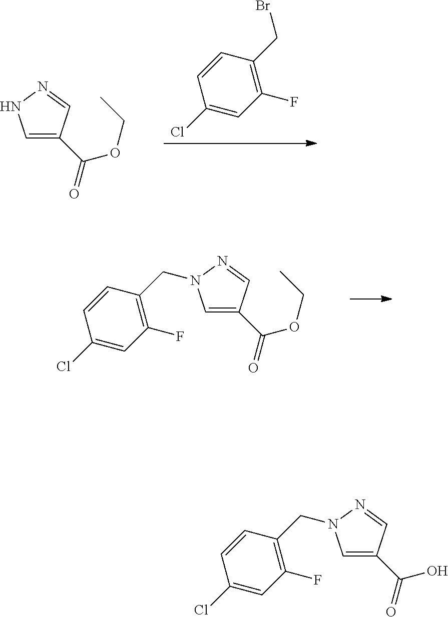

Step 1: ethyl 1-(4-chloro-2-fluorobenzyl)-1H-pyrazole-4-carboxylate To a solution of ethyl 1H-pyrazole-4-carboxylate (1 g, 7.14 mmol) in MeCN (20 mL) was added 1-(bromomethyl)-4-chloro-2-fluorobenzene (1.58 g, 7.14 mmol), K₂CO₃ (1.97 g, 14.28 mmol). The reaction mixture was stirred at room temperature overnight. The mixture was diluted with EtOAc (50 mL), washed with water (50 mL×2), dried over Na₂SO₄, filtered and concentrated to afford ethyl 1-(4-chloro-2-fluorobenzyl)-1H-pyrazole-4-carboxylate (1.9 g, 94%) as a white solid. LCMS m/z=283 [M+H]⁺; ¹H NMR (400 MHz, DMSO-d₆) δ=8.45 (s, 1H), 7.87 (s, 1H), 7.50-7.44 (m, 1H), 7.33-7.25 (m, 2H), 5.42 (s, 2H), 4.21 (q, J=7.1, 2H), 1.26 (d, J=7.1, 3H).

Step 2: 1-(4-chloro-2-fluorobenzyl)-1H-pyrazole-4-carboxylic acid To a solution of (ethyl 1-(4-chloro-2-fluorobenzyl)-1H-pyrazole-4-carboxylate (1.9 g, 6.73 mmol) in a mixture of THF (24 mL), MeOH (6 mL) and water (6 mL) was added NaOH (780 mg, 19.51 mmol). The reaction was stirred at room temperature for 1 h then diluted with water (30 mL) and extracted with EtOAc (60 mL). The aqueous layer was collected and acidified with 1 M HCl to pH˜2 then extracted with EtOAc (60 mL×2). The combined organic layers were washed with brine, dried over Na₂SO₄, filtered and concentrated to afford 1-(4-chloro-2-fluorobenzyl)-1H-pyrazole-4-carboxylic acid (1.39 g, 90%) as a white solid. LCMS m/z=255 [M+H]⁺; ¹H NMR (400 MHz, DMSO-d₆) 5=8.35 (s, 1H), 7.81 (s, 1H), 7.50-7.44 (m, 1H), 7.31-7.26 (m, 2H), 5.41 (s, 2H).

### 1-(4-fluoro-2-(trifluoromethyl)benzyl)-1H-pyrazole-4-carboxylic acid

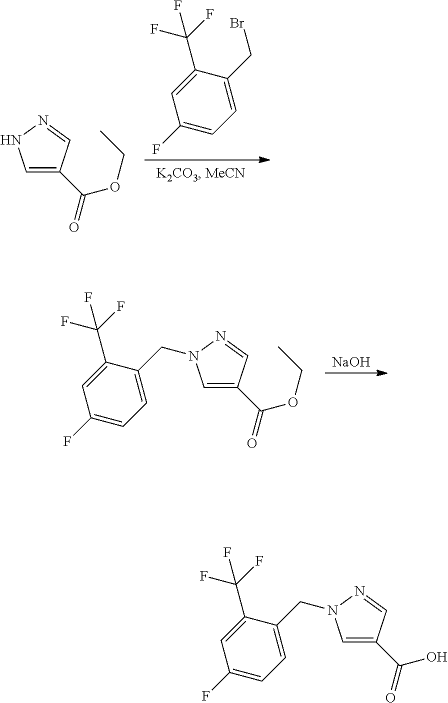

Step 1: ethyl 1-(4-fluoro-2-(trifluoromethyl)benzyl)-1H-pyrazole-4-carboxylate: To a solution of ethyl 1H-pyrazole-4-carboxylate (1.0 g, 7.14 mmol) in MeCN (10 mL) was added K₂CO₃ (2 g, 14.27 mmol) and 1-(bromomethyl)-4-fluoro-2-(trifluoromethyl)benzene (1.8 g, 7.14 mmol). The mixture was stirred at room temperature for 12 h. The reaction was diluted with water (30 mL) and extracted with EtOAc (30 mL×2). The combined organic layers were washed with brine, dried over Na₂SO₄, filtered and concentrated to afford ethyl 1-(4-fluoro-2-(trifluoromethyl)benzyl)-1H-pyrazole-4-carboxylate (1.8 g, 79%) as a white solid. LCMS m/z=317.1[M+H]⁻; ¹H NMR (400 MHz, DMSO-d₆) δ 8.45 (s, 1H), 7.91 (s, 1H), 7.68 (dd, J=9.2, 2.8 Hz, 1H), 7.56-7.51 (m, 1H), 7.17 (dd, J=8.8, 5.4 Hz, 1H), 5.55 (s, 2H), 4.21 (q, J=7.1 Hz, 2H), 1.26 (t, J=7.1 Hz, 3H).

Step 2: 1-(4-fluoro-2-(trifluoromethyl)benzyl)-1H-pyrazole-4-carboxylic acid To a solution of ethyl 1-(4-fluoro-2-(trifluoromethyl)benzyl)-1H-pyrazole-4-carboxylate (5.6 g, 17.7 mmol) in a mixture of THF and H₂O and EtOH (40 mL/10 mL/10 mL) was added NaOH (2.1 g, 53.12 mmol). The reaction mixture was stirred at room temperature overnight. The mixture was diluted with water (100 mL) and extracted with EtOAc (50 mL×3). The aqueous layer was collected and acidified with 1 M HCl to pH˜3 and extracted with EtOAc (50 mL×3). The combined organic layers were washed with brine, dried over Na₂SO₄, filtered and concentrated to afford 1-(4-fluoro-2-(trifluoromethyl)benzyl)-1H-pyrazole-4-carboxylic acid (4.4 g, 86%) as a white solid. LCMS m/z=289.1[M+H]⁺; ¹H NMR (400 MHz, DMSO-d₆) δ 8.36 (s, 1H), 7.86 (s, 1H), 7.68 (dd, J=9.2, 2.8 Hz, 1H), 7.54 (td, J=8.5, 2.8 Hz, 1H), 7.15 (dd, J=8.7, 5.4 Hz, 1H), 5.54 (s, 2H).

### 1-Bromo-3-(bromomethyl)-2-(difluoromethoxy)benzene

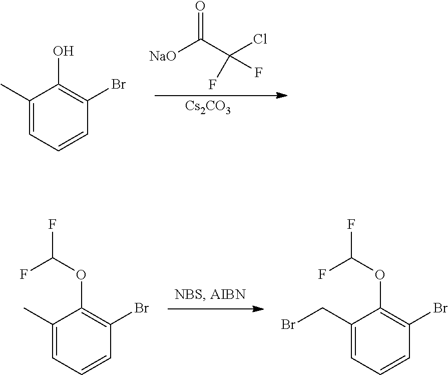

Step 1: 1-bromo-2-(difluoromethoxy)-3-methylbenzene To a solution of 2-bromo-6-methylphenol (1.5 g, 8.23 mmol) in a mixture of DMF (9 mL), H₂O (1 mL) was added sodium 2-chloro-2,2-difluoroacetate (3.18 g, 20.58 mmol) and Cs₂CO₃ (5.36 g, 16.47 mmol). The reaction stirred at 100° C. for 4 h. The mixture was diluted with water (100 mL) and extracted with EtOAc (120 mL×3). The combined organic layers were washed with brine, dried over Na₂SO₄, filtered and concentrated. The residue obtained was purified by column chromatography (Petroleum ether:EtOAc=8:1) to afford 1-bromo-2-(difluoromethoxy)-3-methylbenzene (1.56 g, 82.3%) as a yellow oil. ¹H NMR (DMSO, 400 MHz) δ 7.57 (dd, J=8.0, 1.5 Hz, 1H), 7.34 (dd, J=7.6, 1.5 Hz, 1H), 7.17 (t, J=7.8 Hz, 1H), 7.26-6.74 (m, 1H), 2.31 (s, 3H).

Step 2: 1-bromo-3-(bromomethyl)-2-(difluoromethoxy)benzene To a solution of 1-bromo-2-(difluoromethoxy)-3-methylbenzene (500 mg, 2.12 mmol) in CCl₄ (6 mL) was added NBS (566 mg, 3.18 mmol) and AIBN (139 mg, 0.85 mmol). The reaction mixture stirred at 80° C. for 3 h. The mixture was diluted with water (60 mL) and extracted with EtOAc (80 mL×3). The combined organic layers were washed with brine, dried over Na₂SO₄, filtered and concentrated. The residue obtained was purified by prep-TLC (Petroleum ether:EtOAc=20:1) to afford 1-bromo-3-(bromomethyl)-2-(difluoromethoxy)benzene (283 mg, 43.7%) as a yellow oil. ¹H NMR (DMSO, 400 MHz) δ 7.75 (dd, J=8.0, 1.6 Hz, 1H), 7.61 (dd, J=7.7, 1.6 Hz, 1H), 7.31-7.27 (m, 11H), 7.13 (s, 1H), 4.70 (s, 2H).

### 4,4,5,5-tetramethyl-2-(spiro[3.5]non-6-en-7-yl)-1,3,2-dioxaborolane

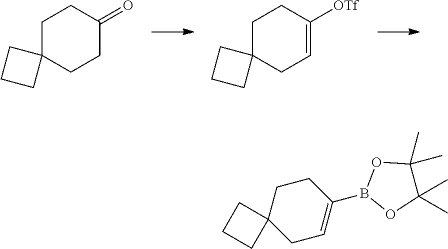 [SMILES: O=C1CCC2(CCC2)CC1 ; O=S(=O)(OC1=CCC2(CCC2)CC1)C(F)(F)F ; CC1(C)OB(C2=CCC3(CCC3)CC2)OC1(C)C]

Step 1: spiro[3.5]non-6-en-7-yl trifluoromethanesulfonate To a solution of spiro[3.5]nonan-7-one (500 mg, 3.6 mmol) and 2,6-di-tert-butyl-4-methylpyridine (1.1 g, 5.4 mmol) in DCM was added trifluoromethanesulfonic anhydride (1.2 g, 4.3 mmol) at 0 aC. The mixture was stirred at room temperature for 3 h. The mixture was concentrated and purified by column chromatography on silica gel (eluent: Pet. Ether) to afford spiro[3.5]non-6-en-7-yl trifluoromethanesulfonate (440 mg, 45% yield) as a yellow oil. ¹H NMR (400 MHz, Chloroform-d) δ 5.67-5.63 (m, 1H), 2.37-2.30 (m, 2H), 2.26-2.21 (m, 2H), 1.90-1.75 (m, 8H).

Step 2: 4,4,5,5-tetramethyl-2-(spiro[3.5]non-6-en-7-yl)-1,3,2-dioxaborolane To a solution of spiro[3.5]non-6-en-7-yl trifluoromethanesulfonate (200 mg, 0.74 mmol) in 1,4-dioxane (4 mL) and H₂O (1 mL) was added 4,4,4′,4′,5,5,5′,5′-octamethyl-2,2′-bi(1,3,2-dioxaborolane) (188 mg, 0.89 mmol), KOAc (217 mg, 2.22 mmol) and Pd(dppf)Cl₂ (54 mg, 0.074 mmol). The reaction was stirred at 100° C. under N₂ atmosphere for 2 h. The mixture was diluted with water (10 mL) and extracted with EtOAc (20 mL×2). The combined organic layers were dried over Na₂SO₄, filtered and concentrated. The residue was purified by column chromatography on silica gel (eluent: Pet. Ether) to afford 4,4,5,5-tetramethyl-2-(spiro[3.5]non-6-en-7-yl)-1,3,2-dioxaborolane (100 mg, 53% yield) as a yellow oil. ¹H NMR (400 MHz, Chloroform-d) δ 6.48-6.44 (m, 1H), 2.16-2.11 (m, 4H), 1.86-1.82 (m, 2H), 1.79-1.68 (m, 6H), 1.25 (s, 12H).

### tert-butyl 2-bromo-6-(bromomethyl)benzoate

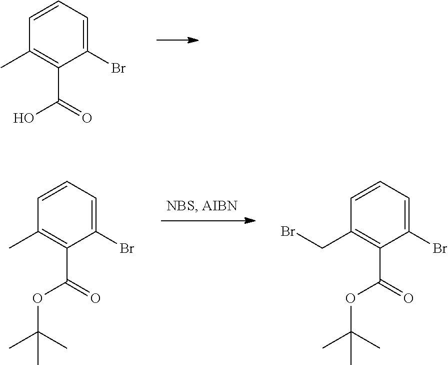 [SMILES: Cc1cccc(Br)c1C(=O)O ; Cc1cccc(Br)c1C(=O)OC(C)(C)C ; CC(C)(C)OC(=O)c1c(Br)cccc1CBr]

Step 1: tert-butyl 2-bromo-6-methylbenzoate To a solution of 2-bromo-6-methylbenzoic acid (10 g, 46 mmol) in anhydrous THF (100 mL) at −78° C. was added tert-butyl 2,2,2-trichloroacetimidate (20.3 g, 93 mmol) and BF₃·Et₂O (13.2 g, 93 mmol). The reaction mixture was stirred at room temperature overnight. The mixture was diluted with H₂O (200 mL), extracted with EtOAc (200 mL×3), dried over Na₂SO₄, filtered and concentrated. The residue was purified by column chromatography on silica gel (eluent: Pet. Ether:EtOAc=30:1, v/v) to afford tert-butyl 2-bromo-6-methylbenzoate (8.7 g, 69%) as a yellow oil. ¹H NMR (400 MHz, DMSO-d₆) δ 7.47 (dd, J=7.2, 1.8 Hz, 1H), 7.30-7.23 (m, 2H), 2.29 (s, 3H), 1.55 (s, 9H).

Step 2: tert-butyl 2-bromo-6-(bromomethyl)benzoate To a solution of tert-butyl 2-bromo-6-methylbenzoate (500 mg, 1.85 mmol) in THF (5 mL) was added NBS (165 mg, 0.93 mmol) and AIBN (30 mg, 0.185 mmol). Then the mixture was stirred at 80° C. for 4 h. The mixture was diluted with water (40 mL) and extracted with DCM (80 mL×2). The combined organic layers were dried over Na₂SO₄, filtered and concentrated. The mixture was purified by prep-TLC (eluent: Pet. Ether/EtOAc=20/1, v/v) to afford tert-butyl 2-bromo-6-(bromomethyl)benzoate (227 mg, 35% yield) as a white solid. ¹H NMR (400 MHz, Chloroform-d) δ 7.53-7.49 (m, 1H), 7.38-7.34 (m, 1H), 7.21 (t, J=8.0 Hz, 1H), 4.50 (s, 2H), 1.66 (s, 9H).

### 1-bromo-3-(bromomethyl)-2-(trifluoromethyl)benzene

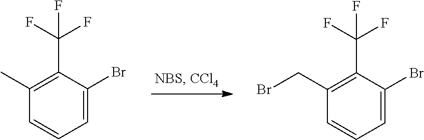

To a solution of 1-bromo-3-methyl-2-(trifluoromethyl)benzene (240 mg, 1.0 mmol) in CCl₄ (2 mL) was added NBS (224 mg, 1.3 mmol) and AIBN (17 mg, 0.1 mmol). The mixture was stirred at 80° C. for 4 h. The reaction was diluted with water (10 mL) and extracted with DCM (20 mL×3). The combined organic layers were washed with brine, dried over Na₂SO₄, filtered concentrated and purified by prep-TLC (Petrol ether:EtOAc=20:1) to afford 1-bromo-3-(bromomethyl)-2-(trifluoromethyl)benzene (170 mg, 53%) as a yellow oil. ¹H NMR (400 MHz, Chloroform-d) δ 7.71 (d, J=7.8 Hz, 1H), 7.44 (d, J=7.8 Hz, 1H), 7.32 (t, J=7.8 Hz, 1H), 4.65 (s, 2H).

### tert-butyl 2-(4,4-difluorocyclohexyl)-6-hydroxybenzoate

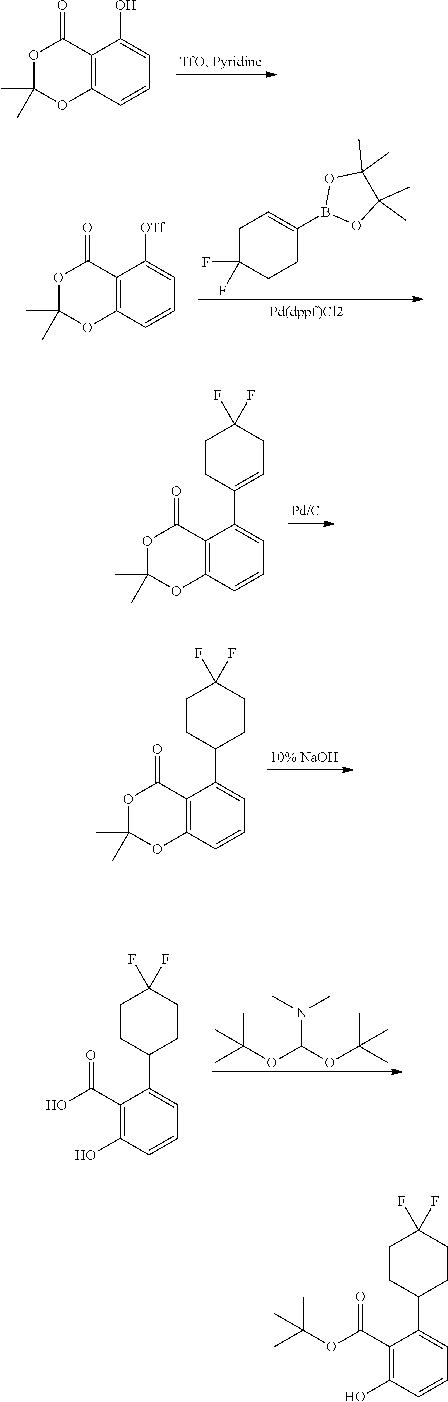

Step 1: 2,2-dimethyl-4-oxo-4H-benzo[d][1,3]dioxin-5-yl trifluoromethanesulfonate To a solution of 5-hydroxy-2,2-dimethyl-4H-benzo[d][1,3]dioxin-4-one (50 mg, 0.25 mmol) in DCM (2 ml) was added pyridine (73 mg, 0.92 mmol) and Tf₂O (87 mg, 0.31 mmol). The mixture was stirred at 0° C. for 2 h. The mixture was diluted with water (30 mL) and extracted with EtOAc (20 mL×3). The organic layer was washed with brine, dried over Na₂SO₄, filtered and concentrated. The crude was purified by prep-TLC (eluent: Pet. Ether:EtOAc=8:1) to afford 2,2-dimethyl-4-oxo-4H-benzo[d][1,3]dioxin-5-yl trifluoromethanesulfonate (54 mg, 64%) as a white solid. LCMS m/z=327.1[M+H]⁺; ¹H NMR (400 MHz, Chloroform-d) δ 7.64-7.56 (m, 1H), 7.09-6.98 (m, 2H), 1.76 (s, 6H).

Step 2: 5-(4,4-difluorocyclohex-1-en-1-yl)-2,2-dimethyl-4H-benzo[d][1,3]dioxin-4-one To a solution of 2,2-dimethyl-4-oxo-4H-benzo[d][1,3]dioxin-5-yl trifluoromethanesulfonate (100 mg, 0.30 mmol) in 1,4-dioxane/H₂O (2 mL/0.5 mL) was added 2-(4,4-difluorocyclohex-1-en-1-yl)-4,4,5,5-tetramethyl-1,3,2-dioxaborolane (75 mg, 0.30 mmol), Pd(dppf)Cl₂ (222 mg, 0.03 mmol), Na₂CO₃ (100 mg, 1.0 mmol). The reaction mixture was stirred at 90° C. overnight under a N₂ atmosphere. The mixture was diluted with water (30 mL) and extracted with DCM (20 mL×3). The organic layer was washed with brine, dired over Na₂SO₄, filtered and concentrated. The crude was purified by prep-TLC (eluent: Pet. Ether:EtOAc=5:1) to afford 5-(4,4-difluorocyclohex-1-en-1-yl)-2,2-dimethyl-4H-benzo[d][1,3]dioxin-4-one (90 mg, 65%) as a white solid. LCMS m/z=295.2[M+H]⁺; ¹H NMR (400 MHz, Chloroform-d) δ 7.49-7.41 (m, 1H), 6.92-6.84 (m, 2H), 5.46-5.39 (m, 1H), 2.77-2.62 (m, 2H), 2.50-2.40 (m, 2H), 2.29-2.15 (m, 2H), 1.73 (s, 6H).

Step 3: 5-(4,4-difluorocyclohexyl)-2,2-dimethyl-4H-benzo[d][1,3]dioxin-4-one: To a solution of 5-(4,4-difluorocyclohex-1-en-1-yl)-2,2-dimethyl-4H-benzo[d][1,3]dioxin-4-one (50 mg, 0.17 mmol) in MeOH (2 mL) was added 50% Pd/C (25 mg). The mixture was stirred at 45° C. for 2 h. The mixture was filtered and concentrated to afford 5-(4,4-difluorocyclohexyl)-2,2-dimethyl-4H-benzo[d][1,3]dioxin-4-one (50 mg, 92%) as a white solid. ¹H NMR (400 MHz, Chloroform-d) δ 7.50-7.43 (m, 1H), 7.10-7.06 (m, 1H), 6.86-6.82 (m, 1H), 4.03-3.92 (m, 1H), 2.26-2.14 (m, 3H), 2.06-1.86 (m, 5H), 1.71 (s, 6H).

Step 4: 2-(4,4-difluorocyclohexyl)-6-hydroxybenzoic acid To a solution of 5-(4,4-difluorocyclohexyl)-2,2-dimethyl-4H-benzo[d][1,3]dioxin-4-one (470 mg, 1.6 mmol) in THF (5 mL) was added 10% NaOH in water (2 mL). The reaction mixture was stirred at 90° C. for 4 h. The mixture was acidified with 1 M HCl to pH˜6 and extracted with DCM (20 mL×3). The combined organic layers were washed with brine, dried over Na₂SO₄, filtered and concentrated to afford 2-(4,4-difluorocyclohexyl)-6-hydroxybenzoic acid (406 mg, 99%) as a white solid. LCMS m/z=255.2[M−H]⁻; ¹H NMR (400 MHz, Chloroform-d) δ 7.44-7.36 (m, 1H), 6.93-6.86 (m, 2H), 3.66-3.55 (m, 1H), 1.99-1.79 (m, 8H).

Step 5: tert-butyl 2-(4,4-difluorocyclohexyl)-6-hydroxybenzoate To a solution of 2-(4,4-difluorocyclohexyl)-6-hydroxybenzoic acid (100 mg, 0.39 mmol) in THF (3 mL) was added 1,1-di-tert-butoxy-N,N-dimethylmethanamine (317 mg, 1.56 mmol) at 90° C. The reaction mixture was stirred at 90° C. for 2 h. The reaction mixture was stirred at 90° C. overnight under a N₂ atmosphere. The mixture was diluted with water (30 mL) and extracted with DCM (20 mL×3). The organic layer were washed with brine, dried over Na₂SO₄, filtered and concentrated. The crude was purified by prep-TLC (eluent: Pet. Ether:EtOAc=20:1) to afford tert-butyl 2-(4,4-difluorocyclohexyl)-6-hydroxybenzoate (60 mg, 50%) as a yellow oil. LCMS m/z=311.2[M−H]⁻; ¹H NMR (400 MHz, Chloroform-d) δ 7.34-7.29 (m, 1H), 6.87-6.82 (m, 2H), 3.50-3.47 (m, 1H), 1.98-1.82 (m, 8H), 1.64 (s, 9H).

### 1-(2-(methoxycarbonyl)benzyl)-1H-pyrazole-4-carboxylic acid

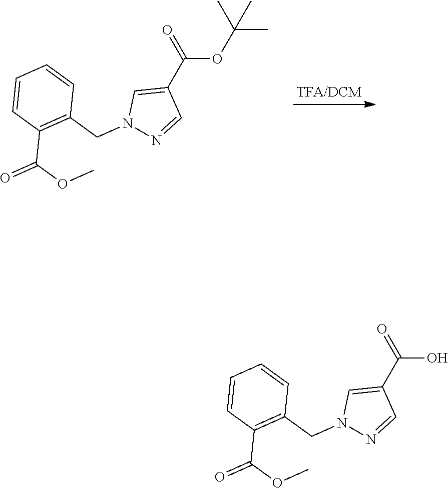 [SMILES: COC(=O)c1ccccc1Cn1cc(C(=O)OC(C)(C)C)cn1 ; COC(=O)c1ccccc1Cn1cc(C(=O)O)cn1]

To a solution of tert-butyl 1-(2-(methoxycarbonyl)benzyl)-1H-pyrazole-4-carboxylate (76 mg, 0.24 mmol) in DCM (2 mL) was added TFA (1 mL). The reaction mixture was stirred at room temperature for 2 h. The solvent was removed under vacuum to afford 1-(2-(methoxycarbonyl)benzyl)-1H-pyrazole-4-carboxylic acid (62 mg, 100%) which was used directly in the next step. LCMS m/z=261.1 [M+H]⁺.

### tert-butyl 2-(bromomethyl)-6-(4,4-difluorocyclohexyl)benzoate

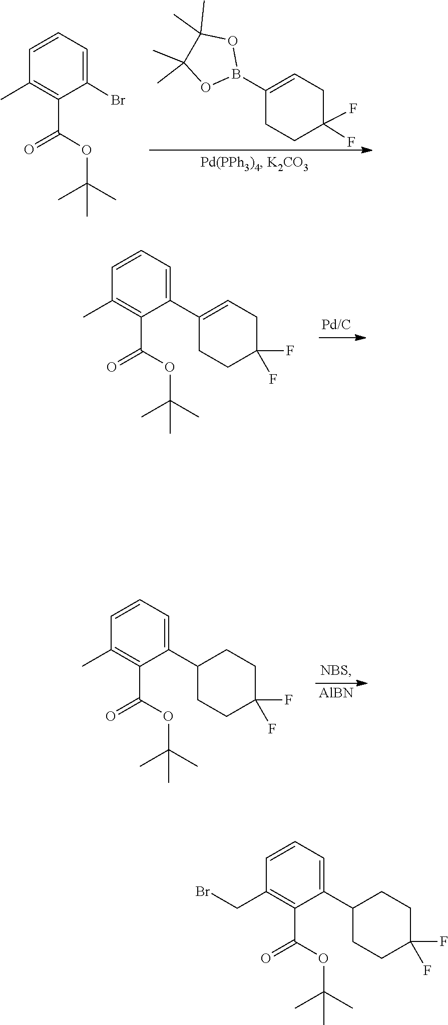

Step 1: tert-butyl 4′,4′-difluoro-3-methyl-2′,3′,4′,5′-tetrahydro-[1,1′-biphenyl]-2-carboxylate To a solution of tert-butyl 2-bromo-6-methylbenzoate (3.5 g, 12.9 mmol) in dioxane (36 mL) and H₂O (12 mL) was added 2-(4,4-difluorocyclohex-1-en-1-yl)-4,4,5,5-tetramethyl-1,3,2-dioxaborolane (3.78 g, 15.5 mmol), Pd(PPh₃)₄ (1.49 g, 1.29 mmol) and K₂CO₃ (3.56 g, 25.8 mmol), the reaction mixture was stirred at 100° C. under N₂ overnight. The mixture was diluted with water (1 mL) and extracted with EtOAc (50 mL×3). The combined organic layers were washed with brine, dried over Na₂SO₄, filtered and concentrated. The residue obtained was purified by column (eluent: PE:EtOAc=200:1) to afford tert-butyl 4′,4′-difluoro-3-methyl-2′,3′,4′,5′-tetrahydro-[1,1′-biphenyl]-2-carboxylate (4 g, 100%) as a white solid. ¹H NMR (400 MHz, Chloroform-d) δ 7.22 (t, J=7.8 Hz, 1H), 7.10 (d, J=7.6 Hz, 1H), 7.01 (d, J=7.6 Hz, 1H), 5.51 (t, J=3.4 Hz, 1H), 2.67-2.57 (m, 4H), 2.36 (s, 3H), 2.20-2.09 (m, 2H), 1.54 (s, 9H).

Step 2: tert-butyl 2-(4,4-difluorocyclohexyl)-6-methylbenzoate To a solution of tert-butyl 4′,4′-difluoro-3-methyl-2′,3′,4′,5′-tetrahydro-[1,1′-biphenyl]-2-carboxylate (4 g, 13 mmol) in MeOH (40 mL) was added 10% Pd/C (1.6 g), the reaction mixture was stirred at 50° C. for 2 h with a H₂ balloon. The mixture was filtered with EtOAc (200 mL) and concentrated to afford tert-butyl 2-(4,4-difluorocyclohexyl)-6-methylbenzoate (3.77 g, 93%) as a white oil. ¹H NMR (400 MHz,) δ 7.23 (t, J=7.8 Hz, 1H), 7.11 (d, J=7.6 Hz, 1H), 7.05 (d, J=7.6 Hz, 1H), 2.68 (m, 1H), 2.33 (s, 3H), 2.21 (q, J=9.2, 7.6 Hz, 2H), 1.96 (d, J=10.4 Hz, 2H), 1.86-1.73 (m, 4H), 1.61 (s, 9H).

Step 3: tert-butyl 2-(bromomethyl)-6-(4,4-difluorocyclohexyl)benzoate To a solution of tert-butyl 2-(4,4-difluorocyclohexyl)-6-methylbenzoate (1 g, 3.22 mmol) in CCl₄ (10 mL) was added NBS (287 mg, 1.61 mmol) and AIBN (53 mg, 0.32 mmol), the reaction mixture was stirred at 80° C. for 4 h. The mixture was diluted with water (20 mL) and extracted with DCM (30 mL×3). The combined organic layers were washed with brine, dried over Na₂SO₄, filtered and concentrated. The residue obtained was purified by prep-TLC (eluent: PE:EtOAc=20:1) to afford tert-butyl 2-(bromomethyl)-6-(4,4-difluorocyclohexyl)benzoate (330 mg, 26%) as a white solid. ¹H NMR (400 MHz, Chloroform-d) δ 7.38-7.26 (m, 2H), 7.24 (d, J=1.4 Hz, 1H), 4.53 (s, 2H), 2.78-2.69 (m, 1H), 2.27-2.18 (q, J=9.2, 7.6 Hz, 2H), 1.97 (d, J=10.4 Hz, 2H), 1.87-1.72 (m, 4H), 1.65 (s, 9H).

### 1-(3,4-difluorobenzyl)-1H-pyrazole-4-carboxylic acid

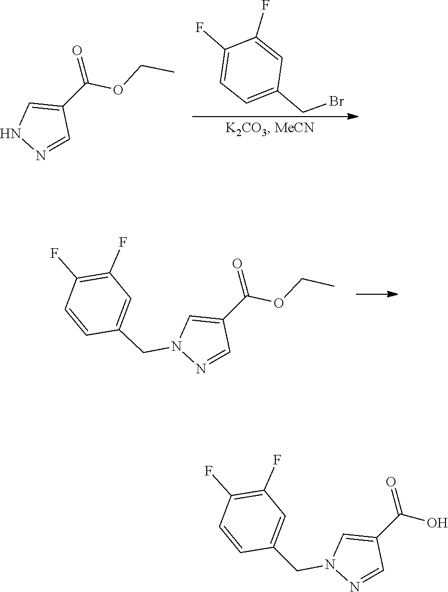

Step 1: ethyl 1-(3,4-difluorobenzyl)-1H-pyrazole-4-carboxylate To a solution of ethyl 1H-pyrazole-4-carboxylate (500 mg, 3.6 mmol) and 4-(bromomethyl)-1,2-difluorobenzene (730 mg, 3.6 mmol) in MeCN was added K₂CO₃ (990 mg, 7.2 mmol). The mixture was stirred at room temperature for 3 h. The mixture was extracted with EtOAc (70 mL×3). The combined organic layers were concentrated to afford ethyl 1-(3,4-difluorobenzyl)-1H-pyrazole-4-carboxylate (920 mg, 83.6% yield) as a white solid. ¹H NMR (400 MHz, DMSO-d₆) δ 8.48 (s, 1H), 7.88 (s, 1H), 7.47-7.35 (m, 2H), 7.16-7.10 (m, 1H), 5.36 (s, 2H), 4.21 (q, J=7.2 Hz, 2H), 1.26 (t, J=7.2 Hz, 4H).

Step 2: 1-(3,4-difluorobenzyl)-1H-pyrazole-4-carboxylic acid To a solution of ethyl 1-(3,4-difluorobenzyl)-1H-pyrazole-4-carboxylate (920 mg, 2.91 mmol) in a mixed solvent of THF, EtOH and H₂O (12 mL, 3 mL and 3 mL) was added NaOH (582 mg, 14.6 mmol). The reaction mixture was stirred at 50° C. for 6 h. The reaction was diluted with water (40 mL) and extracted with EtOAc (40 mL). The aqueous layer was collected and acidified with 1 M HCl to pH˜6 and extracted with EtOAc (70 mL×3). The combined organic layers were washed with brine, dried over Na₂SO₄, filtered and concentrated to afford 1-(3,4-difluorobenzyl)-1H-pyrazole-4-carboxylic acid (520 mg, 75% yield) as a white solid. ¹H NMR (400 MHz, Chloroform-d) δ 8.01 (s, 1H), 7.94 (s, 1H), 7.21-7.13 (m, 1H), 7.11-7.04 (m, 1H), 7.02-6.97 (m, 1H), 5.28 (s, 2H).

### 1-(3,4,5-trifluorobenzyl)-1H-pyrazole-4-carboxylic acid

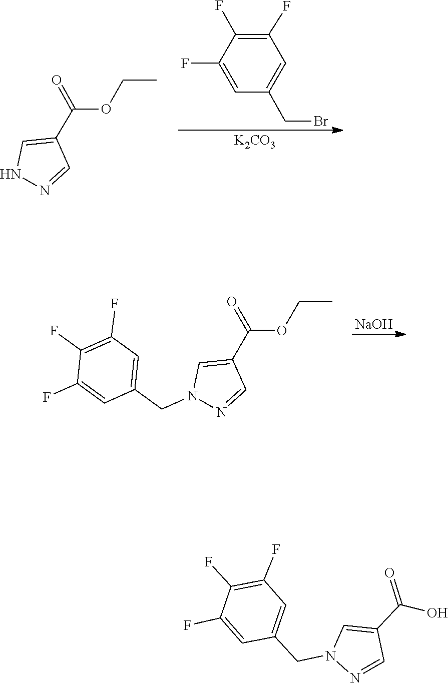

Step 1: ethyl 1-(3,4,5-trifluorobenzyl)-1H-pyrazole-4-carboxylate To a solution of ethyl 1H-pyrazole-4-carboxylate (300 mg, 2.1 mmol), 5-(bromomethyl)-1,2,3-trifluorobenzene (530 mg, 2.3 mmol) and K₂CO₃ (590 mg, 4.2 mmol) in MeCN (5 mL) was stirred at room temperature overnight. The mixture was diluted with EtOAc (50 mL), washed with water (50 mL×2), dried over Na₂SO₄, filtered and concentrated. The residue obtained was purified by column chromatography on silica gel (eluent: Pet. Ether:EtOAc=20:1 to 2:1) to afford ethyl 1-(3,4,5-trifluorobenzyl)-1H-pyrazole-4-carboxylate (580 mg, 95%) as a white solid. LCMS m/z=285.1[M+H]⁻; ¹H NMR (400 MHz, Chloroform-d) δ 7.96 (s, 1H), 7.91 (s, 1H), 6.94-6.77 (m, 2H), 5.24 (s, 2H), 4.29 (q, J=7.2 Hz, 2H), 1.34 (t, J=7.2 Hz, 3H).

Step 2: 1-(3,4,5-trifluorobenzyl)-1H-pyrazole-4-carboxylic acid To a solution of ethyl 1-(3,4,5-trifluorobenzyl)-1H-pyrazole-4-carboxylate (580 mg, 2 mmol) in a mixture of THF (8 mL), EtOH (2 mL) and water (2 mL) was added NaOH (240 mg, 6 mmol). The reaction was stirred at room temperature overnight then diluted with water (30 mL) and extracted with EtOAc (60 mL). The aqueous layer was collected and acidified with 1 M HCl to pH˜2 then extracted with EtOAc (60 mL×2). The combined organic layers were washed with brine, dried over Na₂SO₄, filtered and concentrated to afford 1-(3,4,5-trifluorobenzyl)-1H-pyrazole-4-carboxylic acid (510 mg, 97%) as a white solid. LCMS m/z=257 [M+H]; ¹H NMR (400 MHz, DMSO-d₆) δ 12.37 (s, 1H), 8.41 (s, 1H), 7.84 (s, 1H), 7.58-6.86 (m, 2H), 5.36 (s, 2H).

### tert-butyl 3-(bromomethyl)-4′-fluoro-[1,1′-biphenyl]-2-carboxylate

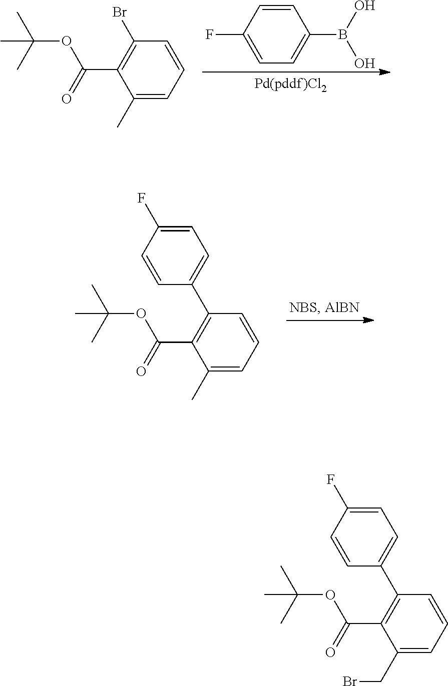

Step 1: tert-butyl 4′-fluoro-3-methyl-[1,1′-biphenyl]-2-carboxylate To a solution of tert-butyl 2-bromo-6-methylbenzoate (2.0 g, 7.4 mmol) in 1,4-dioxane (10 mL) and H₂O (2.5 mL) was added (4-fluorophenyl)boronic acid (1.5 g, 11.1 mmol), K₂CO₃ (3.0 g, 22.2 mmol) and Pd(PPh₃)₄ (850 mg, 0.74 mmol). The reaction was stirred at 100° C. in microwave for 2 h. The mixture was diluted with water (200 mL) and extracted with EtOAc (200 mL×2). The combined organic layers were dried over Na₂SO₄, filtered and concentrated. The residue was purified by column chromatography on silica gel (eluent: Pet. Ether/EtOAc=40/1, v/v) to afford tert-butyl 4′-fluoro-3-methyl-[1,1′-biphenyl]-2-carboxylate (1.9 g, 90%) as a yellow oil. ¹HNMR (400 MHz, Methanol-d₄) δ 7.36-7.32 (m, 3H), 7.24-7.22 (m, 1H), 7.14-7.10 (m, 3H), 2.37 (s, 3H), 1.31 (s, 9H).

Step 2: tert-butyl 3-(bromomethyl)-4′-fluoro-[1,1′-biphenyl]-2-carboxylate To a solution of tert-butyl 4′-fluoro-3-methyl-[1,1′-biphenyl]-2-carboxylate (1.8 g, 6.29 mmol) in CCl₄ (10 mL) was added NBS (2.23 g, 12.58 mmol) and AIBN (99 mg, 0.6 mmol). The mixture was stirred at 80° C. for 4 h. The mixture was diluted with water (100 mL) and extracted with DCM (100 mL×3). The combined organic layers were washed with brine, dried over Na₂SO₄, filtered and concentrated. The crude was purified by column chromatography on silica gel (eluent: Pet. Ether/EtOAc=30/1, v/v) to afford tert-butyl 3-(bromomethyl)-4′-fluoro-[1,1′-biphenyl]-2-carboxylate (700 mg, 32%) as a yellow oil. ¹H NMR (400 MHz, Methanol-d₄) δ 7.52-7.45 (m, 2H), 7.30-7.42 (m, 3H), 7.20-7.24 (m, 2H), 4.71 (s, 11H), 1.31 (s, 9H).

### tert-butyl 2-bromo-6-((diethoxyphosphoryl)methyl)benzoate

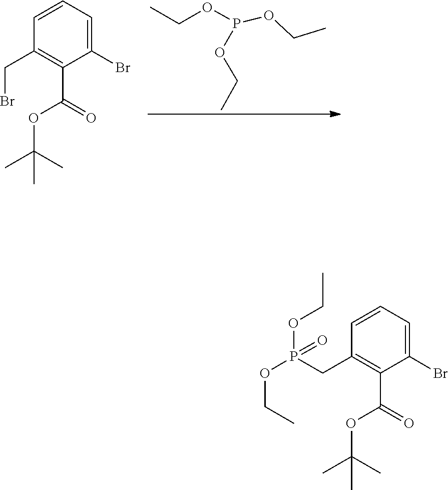

A solution of tert-butyl 2-bromo-6-(bromomethyl)benzoate (100 mg, 0.28 mmol) in triethyl phosphite (1 mL) was stirred at 50° C. overnight. The mixture was concentrated and used to next step without further purification. LCMS m/z=407.2[M+H]⁺; ¹H NMR (400 MHz, DMSO-d6) δ 7.58-7.55 (m, 1H), 7.41 (d, J=2.7 Hz, 1H), 7.37-7.32 (m, 1H), 4.00-3.90 (m, 4H), 3.24 (d, J=22.0 Hz, 2H), 1.59-1.55 (m, 9H), 1.16 (t, J=7.0 Hz, 6H).

### tert-butyl 2-(bromomethyl)-6-(4,4-difluorocyclohexyl)-3-(trifluoromethyl) benzoate

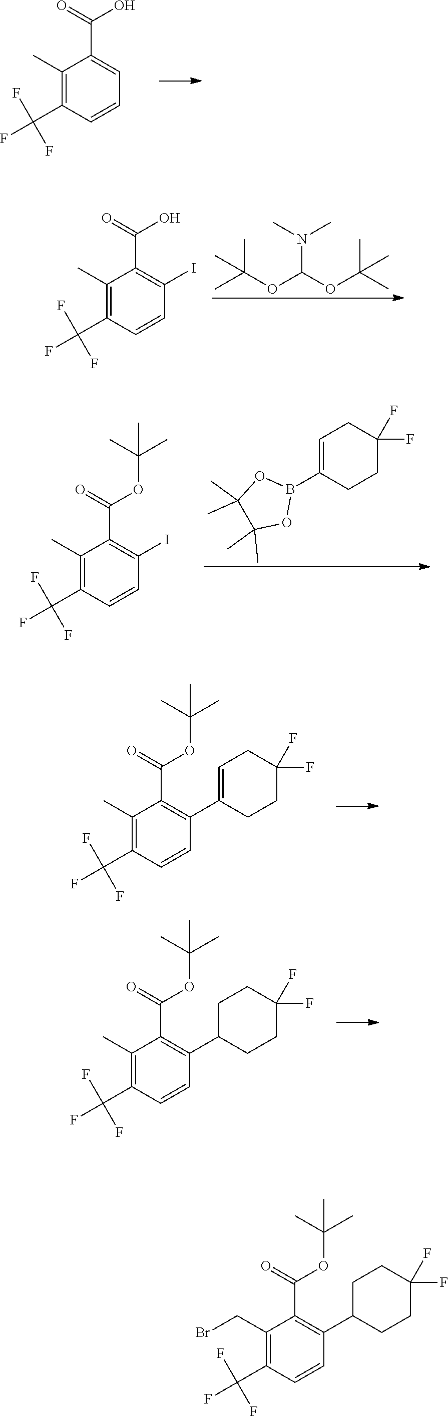

Step 1: 6-iodo-2-methyl-3-(trifluoromethyl)benzoic acid To a solution of 2-methyl-3-(trifluoromethyl)benzoic acid (2 g, 10.0 mmol) in DMF (20 mL) was added NIS (242 mg, 11.0 mmol) and Pd(OAc)₂ (220 mg, 1.0 mmol). The mixture was stirred at 100° C. for 2 h. The reaction was diluted with water (40 mL) and extracted with EtOAc (50 mL×3). The combined organic layers were washed with brine, dried over Na₂SO₄, filtered concentrated to afford 6-iodo-2-methyl-3-(trifluoromethyl)benzoic acid (5.6 g) as a yellow oil and used directly at next step. ¹H NMR (400 MHz, DMSO-d₆) δ 7.93 (d, J=8.2 Hz, 1H), 7.45 (d, J=8.3 Hz, 1H), 2.39 (s, 3H).

Step 2: tert-butyl 6-iodo-2-methyl-3-(trifluoromethyl)benzoate: To a solution of 6-iodo-2-methyl-3-(trifluoromethyl)benzoic acid (2.27 g, 6.9 mmol) in dry THF (20 mL) was added 1,1-di-tert-butoxy-N,N-dimethylmethanamine (5.6 g, 27.6 mmol). The reaction mixture was stirred at 90° C. for 3 h. The mixture was diluted with water (30 mL) and extracted with EtOAc (20 mL×3). The combined organic layers were washed with brine, dried over Na₂SO₄, filtered and concentrated and purified by prep-TLC (eluting with Petrol ether/EtOAc=50/1 to 30/1) to afford tert-butyl 6-iodo-2-methyl-3-(trifluoromethyl)benzoate (1.1 g, 41%) as a colorless solid. 1H NMR (400 MHz, DMSO-d₆) δ 7.93 (d, J=8.2 Hz, 1H), 7.46 (d, J=8.2 Hz, 1H), 2.38 (s, 3H), 1.58 (s, 9H).

Step 3: tert-butyl 4′,4′-difluoro-3-methyl-4-(trifluoromethyl)-2′,3′,4′,5′-tetrahydro-[1,1′-biphenyl]-2-carboxylate To a solution of tert-butyl 6-iodo-2-methyl-3-(trifluoromethyl)benzoate (1 g, 2.6 mmol) in a mixture of dioxane and H₂O (20 mL/5 mL) was added 2-(4,4-difluorocyclohex-1-en-1-yl)-4,4,5,5-tetramethyl-1,3,2-dioxaborolane (760 mg, 3.1 mmol), Pd(dppf)Cl₂ (190 mg, 0.26 mmol), K₂CO₃ (720 mg, 5.2 mmol) and the mixture was stirred at 100° C. for 2 h under N₂. The mixture was diluted with water (30 mL) and extracted with EtOAc (30 mL×3), the combined organic layers were washed with brine, dried over Na₂SO₄, filtered and concentrated. The residue was purified by column (eluting with Petrol ether/EtOAc=50/1 to =30/1) to afford tert-butyl 4′,4′-difluoro-3-methyl-4-(trifluoromethyl)-2′,3′,4′,5′-tetrahydro-[1,1′-biphenyl]-2-carboxylate (750 mg, 75%) as a colorless oil. ¹H NMR (400 MHz, DMSO-d₆) δ 7.72 (d, J=8.2 Hz, 1H), 7.36 (d, J=8.2 Hz, 1H), 5.51 (s, 1H), 2.65 (m, 2H), 2.56-2.51 (m, 2H), 2.36 (s, 3H), 2.22-2.10 (m, 2H), 1.49 (s, 9H).

Step 4: tert-butyl 6-(4,4-difluorocyclohexyl)-2-methyl-3-(trifluoromethyl)benzoate To a solution of tert-butyl 4′,4′-difluoro-3-methyl-4-(trifluoromethyl)-2′,3′,4′,5′-tetrahydro-[1,1′-biphenyl]-2-carboxylate (750 mg, 2 mmol) in MeOH (6 mL) was added Pd/C (300 mg) and Pd(OH)₂ (300 mg). The reaction mixture was stirred at 50° C. for 3 h under H₂. The solvent was filtered and concentrated under vacuum to afford tert-butyl 6-(4,4-difluorocyclohexyl)-2-methyl-3-(trifluoromethyl)benzoate (700 mg, 93% yield) as a colorless oil. ¹H NMR (400 MHz, DMSO-d₆) δ 7.69 (d, J=8.3 Hz, 1H), 7.44 (d, J=8.3 Hz, 1H), 2.63 (t, J=3.2 Hz, 1H), 2.34 (s, 3H), 2.20-2.10 (m, 2H), 1.91-1.68 (m, 6H), 1.58 (s, 9H).

Step 5: tert-butyl 2-(bromomethyl)-6-(4,4-difluorocyclohexyl)-3-(trifluoromethyl)benzoate To a solution of tert-butyl 6-(4,4-difluorocyclohexyl)-2-methyl-3-(trifluoromethyl)benzoate (600 mg, 1.6 mmol) in CCl₄ (12.5 mL) was added NBS (282 mg, 1.6 mmol) and AIBN (54 mg, 0.3 mmol). The reaction mixture was stirred at 80° C. for 3 h. The mixture was diluted with water (30 mL) and extracted with EtOAc (30 mL×3), the combined organic layers were washed with brine, dried over Na₂SO₄, filtered and concentrated and purified by prep-TLC (Petrol ether/EtOAc=20/1) to afford tert-butyl 2-(bromomethyl)-6-(4,4-difluorocyclohexyl)-3-(trifluoromethyl)benzoate (240 mg, 33%) as a colorless oil. ¹H NMR (400 MHz, DMSO-d₆) δ 7.80 (t, 1H), 7.66 (t, J=2.6 Hz, 1H), 4.65 (d, J=5.5 Hz, 2H), 2.69 (s, 1H), 2.17 (s, 2H), 1.90-1.73 (m, 6H), 1.63 (d, J=5.6 Hz, 9H).

### tert-butyl 6-(bromomethyl)-2-(4,4-difluorocyclohexyl)-3-fluorobenzoate

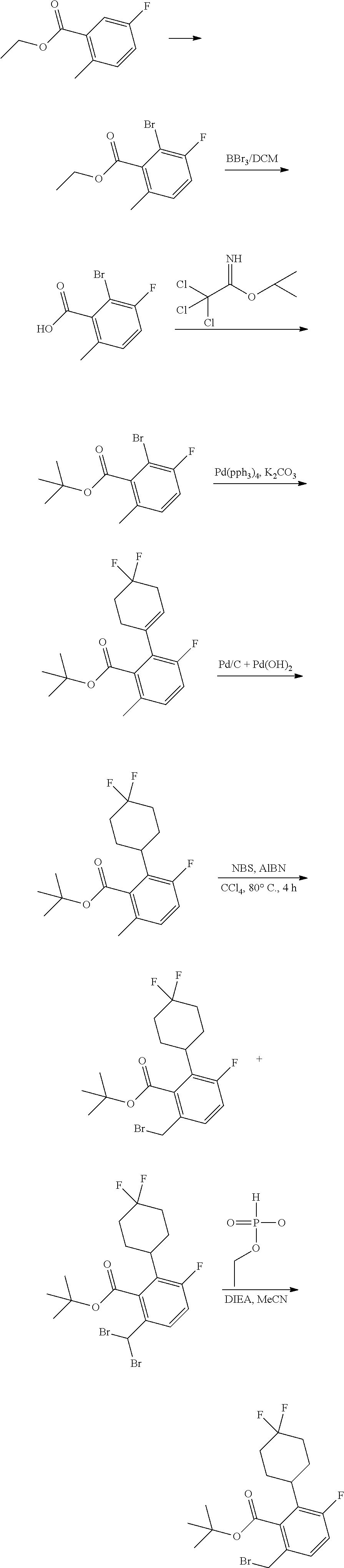

Step 1: ethyl 2-bromo-3-fluoro-6-methylbenzoate To a solution of ethyl 5-fluoro-2-methylbenzoate (5.0 g, 27.5 mmol) in DCE (30 mL) were added Na₂S₂O₈ (13.0 g, 55 mmol), NBS (2.38 g, 30 mmol), Pd(OAc)₂ (300 mg, 1.375 mmol) and TfOH (7 mL). The reaction mixture was stirred at 80° C. for 4 h. The mixture was diluted with water (30 mL) and extracted with EtOAc (50 mL×3), the combined organic layers were washed with brine, dried over Na₂SO₄, filtered and concentrated. The residue was purified by column chromatography on silica gel (eluent: Pet. Ether/EtOAc=40/1) to afford ethyl 2-bromo-3-fluoro-6-methylbenzoate (6.0 g, 80%). ¹H NMR (400 MHz, DMSO-d₆) δ 7.41-7.32 (m, 2H), 4.38 (d, J=7.2 Hz, 2H), 2.25 (d, J=1.4 Hz, 3H), 1.32 (s, 3H).

Step 2: 2-bromo-3-fluoro-6-methylbenzoic acid To a solution of ethyl 2-bromo-3-fluoro-6-methylbenzoate (3 g, 11.49 mmol) in DCM (30 mL) was added BBr₃ (5.76 g, 22.98 mmol) and the mixture stirred at room temperature overnight. The mixture was diluted with water (50 mL), extracted with DCM (50 mL×3) and the combined organic layers washed with brine, dried over Na₂SO₄ and filtered. The solvent was removed and the residue purified by column chromatography on silica gel (eluent: Pet.ether:EtOAc=6:1) to afford the 2-bromo-3-fluoro-6-methylbenzoic acid (1.93 g, 72%) as a white solid. ¹H NMR (400 MHz, DMSO-d₆) δ 13.83 (s, 1H), 7.32 (d, J=7.0 Hz, 2H), 2.28 (s, 3H).

Step 3: tert-butyl 2-bromo-3-fluoro-6-methylbenzoate To a solution of 2-bromo-3-fluoro-6-methylbenzoic acid (1.88 g, 8.06 mmol) in dry THF (20 mL) were added tert-butyl 2,2,2-trichloroacetimidate (3.5 g, 16.12 mmol) and BF₃·Et₂O (1.7 g, 12.09 mmol) at 0° C. The mixture was stirred at room temperature overnight. The mixture was diluted with water (50 mL), extracted with EtOAc (30 mL×3) and the combined organic layers washed with brine, dried over Na₂SO₄ and filtered. The solvent was removed and the residue purified by column chromatography on silica gel (eluent: Pet.ether:EtOAc=30:1) to afford the tert-butyl 2-bromo-3-fluoro-6-methylbenzoate (935 mg, 40%) as a yellow oil. ¹H NMR (400 MHz, DMSO-d₆) δ 7.35-7.30 (m, 2H), 2.27 (s, 3H), 1.56 (s, 9H).

Step 4: tert-butyl 4′,4′,6-trifluoro-3-methyl-2′,3′,4′,5′-tetrahydro-[1,1′-biphenyl]-2-carboxylate To a solution of tert-butyl 2-bromo-3-fluoro-6-methylbenzoate (200 mg, 0.69 mmol) in dioxane/H₂O (3 mL/1 mL) were added 2-(4-(difluoromethyl)cyclohex-1-en-1-yl)-4,4,5,5-tetramethyl-1,3,2-dioxaborolane (202 mg, 0.83 mmol), K₂CO₃ (191 mg, 1.38 mmol) and Pd(dppf)Cl₂ (50 mg, 0.069 mmol). The reaction mixture was stirred at 100° C. for 2 h then diluted with water (10 mL) and extracted with EtOAc (25 mL×3). The combined organic layers were washed with brine, dried over Na₂SO₄, filtered and concentrated to in vacuo. The residue was purified by prep-TLC (eluent: Pet.ether:EtOAc=10:1) to afford the tert-butyl 4′,4′,6-trifluoro-3-methyl-2′,3′,4′,5′-tetrahydro-[1,1′-biphenyl]-2-carboxylate (185 mg, 82%) as a white solid. ¹H NMR (400 MHz, DMSO-d₆) δ 7.25-7.14 (m, 2H), 5.48 (s, 1H), 2.70-2.60 (m, 2H), 2.48-2.43 (m, 2H), 2.23 (s, 3H), 2.19-2.08 (m, 2H), 1.48 (s, 9H).

Step 5: tert-butyl 2-(4,4-difluorocyclohexyl)-3-fluoro-6-methylbenzoate To a solution of tert-butyl 4′,4′,6-trifluoro-3-methyl-2′,3′,4′,5′-tetrahydro-[1,1′-biphenyl]-2-carboxylate (135 mg, 0.4136 mmol) in MeOH (2 mL) were added Pd(OH)₂ (54 mg) and Pd/C (54 mg). The reaction mixture was stirred at 50° C. for 8 h with H₂ balloon. The mixture was filtrated with MeOH and concentrated under vacuum to afford crude tert-butyl 2-((4-(8-(((3-(4-(trifluoromethyl)cyclohexyl)benzyl)oxy)methyl)-2-(1-(trifluoromethyl)cyclopropane-1-carbonyl)-2,6-diazaspiro[3.4]octane-6-carbonyl)-1H-pyrazol-1-yl)methyl)benzoate (55 mg, 100%) which was used directly in the next step. ¹H NMR (400 MHz, DMSO-d₆) δ 7.17-7.09 (m, 2H), 2.60 (t, J=13.0 Hz, 1H), 2.20 (s, 3H), 2.15-1.98 (m, 4H), 1.90-1.75 (m, 4H), 1.56 (s, 9H).

Step 6: tert-butyl 6-(dibromomethyl)-2-(4,4-difluorocyclohexyl)-3-fluorobenzoate: To a solution of tert-butyl 2-(4,4-difluorocyclohexyl)-3-fluoro-6-methylbenzoate (120 mg, 0.365 mmol) in CCl₄ (2 mL) were added NBS (78 mg, 0.438 mmol) and AIBN (12 mg, 0.073 mmol). The reaction mixture was stirred at 70° C. overnight. The mixture was diluted with water (10 mL) and extracted with DCM (25 mL×3). The combined organic layers were washed with brine, dried over Na₂SO₄, filtered and concentrated to in vacuo to afford crude tert-butyl 6-(dibromomethyl)-2-(4,4-difluorocyclohexyl)-3-fluorobenzoate (170 mg) which was used directly in the next step.

Step 7: tert-butyl 6-(bromomethyl)-2-(4,4-difluorocyclohexyl)-3-fluorobenzoate: To a solution of tert-butyl 6-(dibromomethyl)-2-(4,4-difluorocyclohexyl)-3-fluorobenzoate (170 mg, 0.35 mmol) in MeCN (2 mL) were added diethyl phosphonate (48 mg, 0.35 mmol) and DIEA (45 mg, 0.35 mmol). The reaction mixture was stirred at room temperature overnight then diluted with water (10 mL) and extracted with EtOAc (25 mL×3). The combined organic layers were washed with brine, dried over Na₂SO₄, filtered and concentrated to in vacuo. The residue was purified by prep-TLC (eluent: Pet.ether:EtOAc=20:1) to afford the tert-butyl 6-(bromomethyl)-2-(4,4-difluorocyclohexyl)-3-fluorobenzoate (84 mg, 59%) as a white solid. ¹H NMR (400 MHz, DMSO-d₆) δ 7.49 (dd, J₁=8.6 Hz, J₂=5.0 Hz, 1H), 7.30-7.25 (m, 1H), 4.61 (s, 2H), 2.66 (t, J=12.2 Hz, 1H), 2.17-2.02 (m, 4H), 1.91-1.78 (m, 4H), 1.60 (s, 9H).

### tert-butyl 2-(bromomethyl)-6-(4,4-difluorocyclohexyl)-3-fluorobenzoate

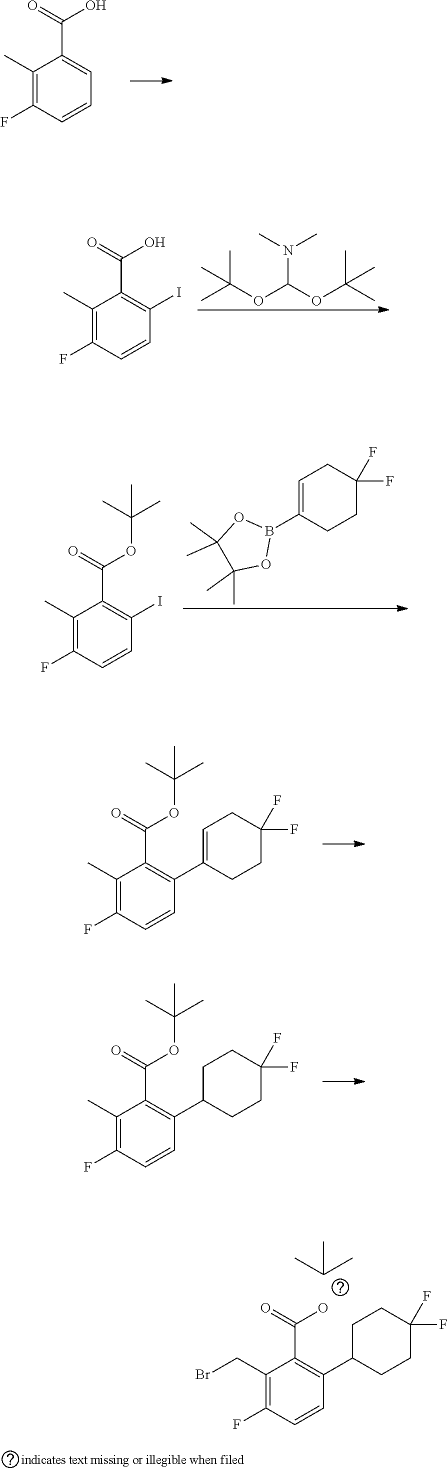

Step 1: 3-fluoro-6-iodo-2-methylbenzoic acid: To a solution of 3-fluoro-2-methylbenzoic acid (5 g, 32.44 mmol) in DMF (25 mL) was added NIS (8.03 g, 35.68 mmol) and Pd(OAc)₂ (728 mg, 3.24 mmol). The reaction mixture was stirred at 100° C. for 2 h. The mixture was diluted with water (250 mL) and extracted with EtOAc (100 mL×3). The combined organic layers were washed with brine, dried over Na₂SO₄, filtered and concentrated to afford crude 3-fluoro-6-iodo-2-methylbenzoic acid (10 g, 100%) as brown oil which was used directly in the next step. ¹H NMR (400 MHz, Chloroform-d) δ 7.63 (dd, J=8.8, 4.8 Hz, 1H), 6.83 (t, J=8.8 Hz, 1H), 2.34 (d, J=2.2 Hz, 3H).

Step 2: tert-butyl 3-fluoro-6-iodo-2-methylbenzoate: To a solution of 3-fluoro-6-iodo-2-methylbenzoic acid (2.1 g, 7.64 mmol) in THF (10 mL) was added 1,1-di-tert-butoxy-N,N-dimethylmethanamine (6.2 g, 30.57 mmol). The reaction mixture was stirred at 90° C. for 3 h. The mixture was diluted with water (250 mL) and extracted with EtOAc (30 mL×3). The combined organic layers were washed with brine, dried over Na₂SO₄, filtered and concentrated. The residue was purified by column chromatography on silica gel (eluent: Pet.ether:EtOAc=30:1) to afford tert-butyl 3-fluoro-6-iodo-2-methylbenzoate (1.33 g, 53%) as a white solid. ¹H NMR (400 MHz, DMSO-d₆) δ 7.71 (dd, J=8.6, 5.0 Hz, 1H), 7.10-7.05 (m, 1H), 2.19 (d, J=2.2 Hz, 3H), 1.57 (s, 9H).

Step 3: tert-butyl 4,4′,4′-trifluoro-3-methyl-2′,3′,4′,5′-tetrahydro-[1,1′-biphenyl]-2-carboxylate: To a solution of tert-butyl 3-fluoro-6-iodo-2-methylbenzoate (500 mg, 1.49 mmol) in dioxane/H₂O (5 mL/1 mL) was added 2-(4,4-difluorocyclohex-1-en-1-yl)-4,4,5,5-tetramethyl-1,3,2-dioxaborolane (435 mg, 1.78 mmol), K₂CO₃ (410.5 mg, 2.97 mmol) and Pd(dppf)Cl₂ (108 mg, 0.15 mmol). The reaction mixture was stirred at 90° C. overnight. The mixture was diluted with water (20 mL) and extracted with EtOAc (30 mL×3). The combined organic layers were washed with brine, dried over Na₂SO₄, filtered and concentrated. The residue was purified by prep-TLC (Pet.ether:EtOAc=20:1) to afford tert-butyl 4,4′,4′-trifluoro-3-methyl-2′,3′,4′,5′-tetrahydro-[1,1′-biphenyl]-2-carboxylate (400 mg, 82%) as a white solid. ¹HNMR (400 MHz, DMSO-d₆) δ 7.24-7.14 (m, 2H), 5.41 (s, 1H), 2.62 (t, 2H), 2.49-2.47 (m, 1H), 2.24-2.00 (m, 6H), 1.48 (s, 9H).

Step 4: tert-butyl 6-(4,4-difluorocyclohexyl)-3-fluoro-2-methylbenzoate: To a solution of tert-butyl 4,4′,4′-trifluoro-3-methyl-2′,3′,4′,5′-tetrahydro-[1,1′-biphenyl]-2-carboxylate (200 mg, 0.61 mmol) in MeOH (4 mL) was added 40% Pd(OH)₂ (80 mg) and 40% Pd/C (80 mg). The reaction mixture was stirred at 50° C. for 6 h with H₂ balloon. The mixture was filtrated with MeOH and concentrated under vacuum to afford crude tert-butyl 6-(4,4-difluorocyclohexyl)-3-fluoro-2-methylbenzoate (200 mg, 99%) as a white solid. ¹H NMR (400 MHz, DMSO-d₆) δ 7.26-7.14 (m, 2H), 2.64-2.54 (m, 1H), 2.14 (s, 1H), 2.14 (s, 3H), 2.03-1.61 (m, 7H), 1.56 (s, 9H).

Step 5: tert-butyl 2-(bromomethyl)-6-(4,4-difluorocyclohexyl)-3-fluorobenzoate: To a solution of tert-butyl 6-(4,4-difluorocyclohexyl)-3-fluoro-2-methylbenzoate (240 mg, 0.73 mmol) in CCl₄ (8 mL) was added NBS (130 mg, 0.73 mmol) and ATBN (24 mg, 0.15 mmol). The reaction mixture was stirred at 80° C. for 4 h. The mixture was diluted with water (20 mL) and extracted with DCM (20 mL×3). The combined organic layers were washed with brine, dried over Na₂SO₄, filtered and concentrated. The residue was purified by prep-TLC (Pet.ether:EtOAc=20:1) to afford tert-butyl 2-(bromomethyl)-6-(4,4-difluorocyclohexyl)-3-fluorobenzoate (93 mg, 37%) as a white solid. ¹H NMR (400 MHz, Methanol-d₄) δ 7.37 (dd, J=8.8, 5.2 Hz, 1H), 7.17 (t, J=9.2 Hz, 1H), 4.56 (d, J=1.6 Hz, 2H), 2.73 (t, J=11.4 Hz, 1H), 2.24-2.13 (m, 2H), 1.96-1.77 (m, 6H), 1.67 (s, 9H).

### tert-butyl 3-(bromomethyl)-4′-fluoro-2′-methoxy-[1,1′-biphenyl]-2-carboxylate

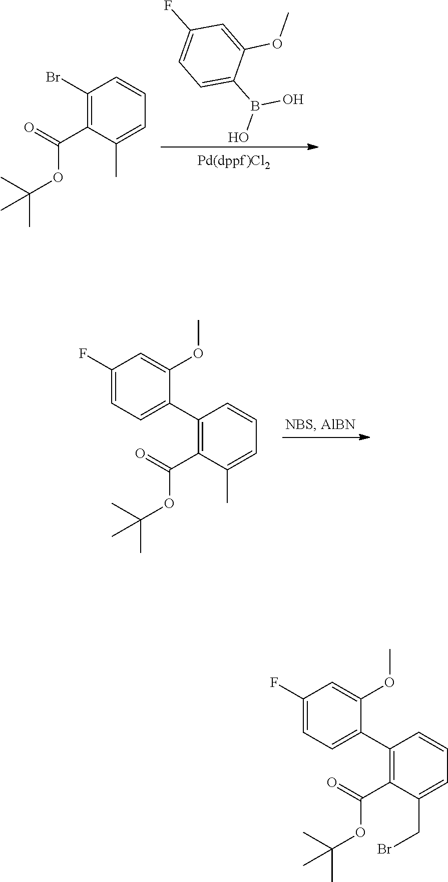

Step 1: tert-butyl 4′-fluoro-2′-methoxy-3-methyl-[1,1′-biphenyl]-2-carboxylate: To a solution of tert-butyl 2-bromo-6-methylbenzoate (500 mg, 1.8 mmol), (4-fluoro-2-methoxyphenyl)boronic acid (315 mg, 1.8 mmol) and K₂CO₃ (510 mg, 3.6 mmol) in dioxane/H₂O (4 mL/1 mL) was added Pd(dppf)₂Cl₂ (135 mg, 0.18 mmol). The mixture was stirred at 90° C. under a N₂ atmosphere overnight. The mixture was diluted with water (200 mL) and extracted with EtOAc (150 mL×2). The combined organic layers were washed with brine, dried over Na₂SO₄, filtered and concentrated. The residue was purified by prep-TLC (Pet ether:EtOAc=20:1) to afford tert-butyl 4′-fluoro-2′-methoxy-3-methyl-[1,1′-biphenyl]-2-carboxylate (398 mg, 68%) as a yellow oil. ¹H NMR (400 MHz, Chloroform-d) δ 7.30 (d, J=7.6 Hz, 1H), 7.19 (d, J=7.6 Hz, 1H), 7.15-7.05 (m, 2H), 6.70-6.65 (m, 2H), 3.75-3.72 (m, 3H), 2.42 (s, 3H), 1.24 (s, 9H).

Step 2: tert-butyl 3-(bromomethyl)-4′-fluoro-2′-methoxy-[1,1′-biphenyl]-2-carboxylate To a solution of tert-butyl 2-bromo-6-methylbenzoate (200 mg, 0.6 mmol) in CCl₄ (2 mL) was added NBS (112 mg, 0.63 mmol) and AIBN (20 mg, 0.12 mmol) The mixture was stirred at 80° C. for 4 h. The mixture was diluted with water (15 mL) and extracted with DCM (20 mL×2). The combined organic layers were washed with brine, dried over Na₂SO₄, filtered and concentrated. The residue was purified by Prep-TLC (Pet ether:EtOAc=20:1) to afford tert-butyl 4′-fluoro-2′-methoxy-3-methyl-[1,1′-biphenyl]-2-carboxylate (50 mg, 20%) as a yellow oil. ¹H NMR (400 MHz, Chloroform-d) δ 7.43-7.35 (m, 2H), 7.21 (m, J=6.3, 2.5 Hz, 1H), 7.15-7.10 (m, 1H), 6.77-6.60 (m, 3H), 4.74 (s, 2H), 3.72 (s, 3H), 1.25 (s, 9H).

### tert-butyl 2-(bromomethyl)-3-chloro-6-(4,4-difluorocyclohexyl)benzoate

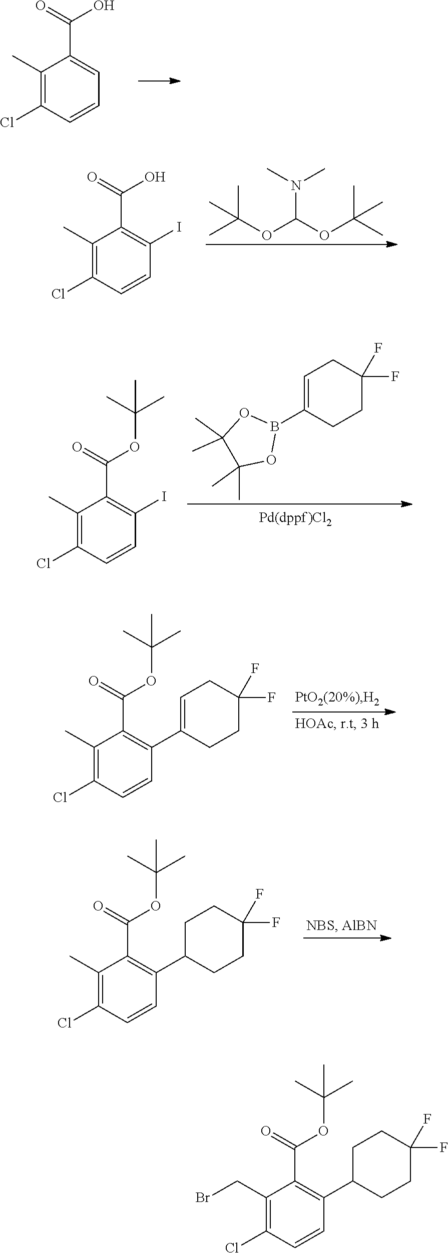

Step 1: 3-chloro-6-iodo-2-methylbenzoic acid To a solution of 3-chloro-2-methylbenzoic acid (2 g, 11.76 mmol) in DMF (14 mL) was added NIS (2.9 g, 12.94 mmol) and Pd(OAc)₂ (264 mg, 1.18 mmol). The reaction mixture was stirred at 100° C. for 2 h. The mixture was diluted with water (80 mL) and extracted with EtOAc (80 mL×2). The combined organic layers were washed with brine, dried over Na₂SO₄, filtered and concentrated to afford 3-chloro-6-iodo-2-methylbenzoic acid (3.5 g), which was used directly in the next step. LCMS m/z=294.75 [M−H]⁻; ¹H NMR (DMSO, 400 MHz) δ 7.70 (d, J=8.4 Hz, 1H), 7.25 (d, J=8.4 Hz, 1H), 2.31 (s, 3H).

Step 2: tert-butyl 3-chloro-6-iodo-2-methylbenzoate To a solution of 3-chloro-6-iodo-2-methylbenzoic acid (3.5 g, 11.83 mmol) in THF (20 mL) was added 1,1-di-tert-butoxy-N,N-dimethylmethanamine (9.6 g, 47.31 mmol) at 90° C. The reaction mixture was stirred at 90° C. for 2 h. The mixture was diluted with water (100 mL) and extracted with EtOAc (120 mL×2). The combined organic layers were washed with brine, dried over Na₂SO₄, filtered and concentrated. The residue obtained was purified by column chromatography on silica gel (eluent: Pet. Ether:EtOAc=80:1 to 50:1) to afford tert-butyl 3-chloro-6-iodo-2-methylbenzoate (2.3 g, 55.3%) as a yellow oil. ¹H NMR (DMSO, 400 MHz) δ 7.70 (d, J=8.4 Hz, 1H), 7.27 (d, J=8.4 Hz, 1H), 2.30 (s, 3H), 1.57 (s, 9H).

Step 3: tert-butyl 4-chloro-4′,4′-difluoro-3-methyl-2′,3′,4′,5′-tetrahydro-[1,1′-biphenyl]-2-carboxylate To a solution of tert-butyl 3-chloro-6-iodo-2-methylbenzoate (500 mg, 1.42 mmol), K₂CO₃ (392 mg, 2.84 mmol) in 1,4-dioxane/water (8 mL, 3/1) was added Pd(dppf)Cl₂ (103 mg, 0.14 mmol) and 2-(4,4-difluorocyclohex-1-en-1-yl)-4,4,5,5-tetramethyl-1,3,2-dioxaborolane (415 mg, 1.70 mmol). The reaction mixture was stirred under a N₂ atmosphere at 100° C. for 2 h. The mixture was diluted with water (30 mL) and extracted with EtOAc (40 mL×2). The combined organic layers were washed with brine, dried over Na₂SO₄, filtered and concentrated, The residue obtained was purified by prep-TLC (Pet. Ether:EtOAc=20:1) to afford tert-butyl 4-chloro-4′,4′-difluoro-3-methyl-2′,3′,4′,5′-tetrahydro-[1,1′-biphenyl]-2-carboxylate (480 mg, 98.2%) as a yellow solid. ¹H NMR (DMSO, 400 MHz) δ 7.47 (d, J=8.4 Hz, 1H), 7.17 (d, J=8.4 Hz, 1H), 5.45 (s, 1H), 2.67-2.58 (m, 2H), 2.46-2.31 (m, 1H), 2.28 (s, 3H), 2.19-2.08 (m, 2H), 2.00 (d, J=7.8 Hz, 1H), 1.48 (s, 9H).

Step 4: tert-butyl 3-chloro-6-(4,4-difluorocyclohexyl)-2-methylbenzoate To a solution of tert-butyl 4-chloro-4′, 4′-difluoro-3-methyl-2′,3,4′,5′-tetrahydro-[1,1′-biphenyl]-2-carboxylate (100 mg, 0.29 mmol) in HOAc (2 mL) was added 20% PtO₂ (20 mg). The reaction mixture was stirred under a H₂ atmosphere at room temperature for 3 h. The mixture was diluted with water (20 mL) and extracted with EtOAc (20 mL×2). The combined organic layers were washed with brine, dried over Na₂SO₄, filtered and concentrated to afford tert-butyl 3-chloro-6-(4,4-difluorocyclohexyl)-2-methylbenzoate (100 mg) as a colorless oil which was used directly in the next step. ¹H NMR (DMSO, 400 MHz) δ 7.44 (d, J=11.2 Hz, 1H), 7.23 (dd, J=12.0, 7.1 Hz, 1H), 2.66-2.55 (m, 1H), 2.26 (s, 3H), 2.12 (s, 2H), 1.97-1.78 (m, 4H), 1.69 (d, J=12.2 Hz, 2H), 1.54 (s, 9H).

Step 5: tert-butyl 2-(bromomethyl)-3-chloro-6-(4,4-difluorocyclohexyl)benzoate: To a solution of tert-butyl 3-chloro-6-(4,4-difluorocyclohexyl)-2-methylbenzoate (100 mg, 0.29 mmol) in CCl₄ (4 mL) was added NBS (52 mg, 0.29 mmol) and AIBN (10 mg, 0.06 mmol). The reaction mixture was stirred at 80° C. for 3 h. The mixture was diluted with water (20 mL) and extracted with EtOAc (20 mL×2). The combined organic layers were washed with brine, dried over Na₂SO₄, filtered and concentrated. The residue obtained was purified by prep-TLC (Pet. Ether:EtOAc=10:1) to afford tert-butyl 2-(bromomethyl)-3-chloro-6-(4,4-difluorocyclohexyl)benzoate (30 mg, 24.6%) as a colorless oil. ¹H NMR (CDCl3, 400 MHz) δ 7.39 (d, J=8.4 Hz, 1H), 7.20 (d, J=8.6 Hz, 1H), 4.60 (s, 2H), 2.64 (s, 1H), 2.22 (s, 2H), 2.03-1.91 (m, 4H), 1.83-1.73 (m, 2H), 1.66 (s, 9H).

### ethyl 1-(3-(bromomethyl)phenyl)cyclohexane-1-carboxylate

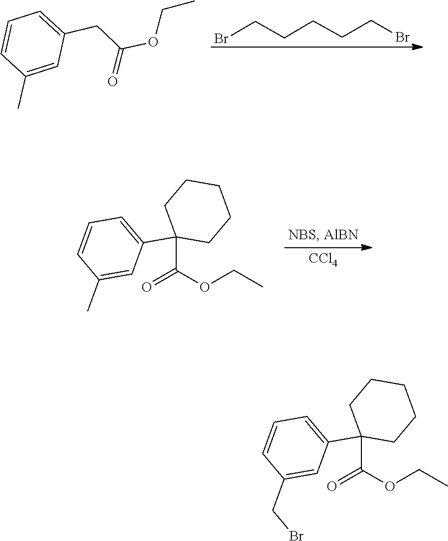 [SMILES: CCOC(=O)Cc1cccc(C)c1 ; BrCCCCCBr ; CCOC(=O)C1(c2cccc(C)c2)CCCCC1 ; "CCl4" ; CCOC(=O)C1(c2cccc(CBr)c2)CCCCC1]

Step 1: ethyl 1-(m-tolyl)cyclohexane-1-carboxylate Sodium hydride (670 mg, 16.86 mmol) was added to a solution of ethyl 2-(m-tolyl)acetate (1.5 g, 8.43 mmol) and 18-crown-6 (667 mg, 2.53 mmol) in DMF (15 mL). The reaction was stirred for 25 minutes and 1,5-dibromopentane (1.2 mL, 9.27 mmol) was added dropwise. The mixture was stirred at room temperature for 18 h. The mixture was diluted with water (200 mL) and extracted with EtOAc (200 mL×2). The combined organic layers were dried over Na₂SO₄, filtered and concentrated. The residue was purified by column chromatography on silica gel (eluent: Pet. Ether/EtOAc=100/1, v/v) to afford ethyl 1-(m-tolyl)cyclohexane-1-carboxylate (240 mg, 12%) as a yellow oil. ¹H NMR (400 MHz, DMSO-d₆) δ 7.21 (t, J=7.6 Hz, 1H), 7.16-7.10 (m, 2H), 7.04 (d, J=7.4 Hz, 1H), 4.06 (q, J=7.2 Hz, 2H), 2.34 (d, J=12.6 Hz, 2H), 2.29 (s, 3H), 1.84-1.78 (m, 1H), 1.62 (t, J=5.2 Hz, 2H), 1.53-1.46 (m, 1H), 1.43-1.31 (m, 3H), 1.24 (q, J=6.2, 4.6 Hz, 1H), 1.11 (t, J=7.2 Hz, 3H).

Step 2: ethyl 1-(3-(bromomethyl)phenyl)cyclohexane-1-carboxylate To a solution of ethyl 1-(m-tolyl)cyclohexane-1-carboxylate (240 mg, 0.98 mmol) in CCl₄ (4 mL) was added NBS (173 mg, 0.98 mmol) and AIBN (32 mg, 0.195 mmol). The mixture was stirred at 80° C. for 4 h. The mixture was diluted with water (50 mL) and extracted with DCM (100 mL×2). The combined organic layers were washed with brine, dried over Na₂SO₄, filtered and concentrated. The residue was purified by prep-TLC (Pet ether:EtOAc=10:1) to afford ethyl 1-(3-(bromomethyl)phenyl)cyclohexane-1-carboxylate (180 mg, 57%) as a yellow oli. ¹H NMR (400 MHz, DMSO-d₆) δ 7.47-7.26 (m, 4H), 4.70 (s, 2H), 4.07 (q, J=7.0 Hz, 2H), 2.35 (d, J=12.6 Hz, 2H), 1.64 (q, J=9.2 Hz, 4H), 1.44-1.32 (m, 2H), 1.27 (d, J=15.8 Hz, 2H), 1.11 (t, J=7.2 Hz, 3H).

### methyl (R)-1-(3-(bromomethyl)phenyl)piperidine-2-carboxylate

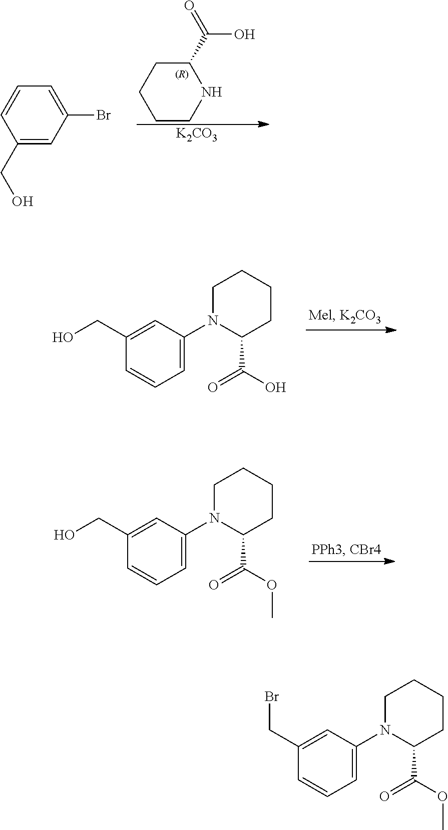

Step 1: (R)-1-(3-(hydroxymethyl)phenyl)piperidine-2-carboxylic acid To a solution of (3-bromophenyl)methanol (200 mg, 1.07 mmol) in DMF (2 mL) was added K₂CO₃ (592 mg, 4.28 mmol), (R)-piperidine-2-carboxylic acid (276 mg, 2.14 mmol) and CuI (41 mg, 0.2 mmol). The mixture was stirred at 140° C. for 1.5 h in microwave. The reaction solution was quenched with water and extracted with EtOAc, the aqueous layer was collected and acidified with 1 M HCl to pH˜2 and extracted with EtOAc (30 mL×3). The combined organic layers were washed with brine, dried over Na₂SO₄, filtered and concentrated to afford (R)-1-(3-(hydroxymethyl)phenyl)piperidine-2-carboxylic acid (600 mg, 100%) as a red oil. LCMS m/z=236.3[M+H]; ¹H NMR (400 MHz, DMSO-d₆) δ 7.10 (1, J=7.8 Hz, 1H), 6.82 (d, J=2.4 Hz, 1H), 6.71 (dd, J=8.2, 2.6 Hz, 1H), 6.65 (d, J=7.4 Hz, 1H), 4.55-4.51 (m, 1H), 4.41 (s, 2H), 3.88 (d, J=10.4 Hz, 1H), 2.14-2.05 (m, 2H), 1.71-1.53 (m, 6H).

Step 2: methyl (R)-1-(3-(hydroxymethyl)phenyl)piperidine-2-carboxylate To a solution of (R)-1-(3-(hydroxymethyl)phenyl)piperidine-2-carboxylic acid (700 mg, 2.98 mmol) in DMF (5.0 mL) was added Mel (1.27 g, 8.93 mmol) and K₂CO₃ (822 mg, 5.95 mmol). The resulting mixture was stirred at room temperature for 4 h. The mixture was diluted with water (100 mL) and extracted with EtOAc (80 mL×3). The combined organic layers were washed with brine, dried over Na₂SO₄, filtered and concentrated. The residue obtained was purified by RP-column (H₂O/MeCN=40/60) to afford methyl (R)-1-(3-(hydroxymethyl)phenyl)piperidine-2-carboxylate (250 mg, 33%) as a red oil. LCMS m/z=250.3[M+H]⁺; ¹H NMR (400 MHz, DMSO-d₆) δ 7.09 (d, J=7.8 Hz, 1H), 6.83 (s, 1H), 6.72 (dd, J=8.2, 2.6 Hz, 1H), 6.67 (d, J=7.4 Hz, 1H), 5.05 (s, 1H), 4.66 (d, J=3.2 Hz, 1H), 4.41 (d, J=5.6 Hz, 2H), 3.57 (s, 3H), 2.11-1.47 (m, 8H).

Step 3: methyl (R)-1-(3-(bromomethyl)phenyl)piperidine-2-carboxylate a solution of methyl (R)-1-(3-(hydroxymethyl)phenyl)piperidine-2-carboxylate (750 mg, 3.01 mmol) in DCM (9 mL) was added PPh₃ (1.2 g, 4.51 mmol) and CBr₄ (1.5 g, 4.51 mmol) in CAN (3 mL) dropwise. The mixture was stirred at 30° C. overnight. The mixture was diluted with water (20 mL) and extracted with DCM (30 mL×3). The combined organic layers were washed with brine, dried over Na₂SO₄, filtered and concentrated. The residue obtained was purified by column chromatography on silica gel (eluent: DCM/MeOH=50:1) to afford methyl (R)-1-(3-(bromomethyl)phenyl)piperidine-2-carboxylate (780 mg, 83%) as a red oil. LCMS m/z=312.2[M+H]⁺; ¹H NMR (400 MHz, DMSO-d₆) δ 7.14 (s, 1H), 6.99-6.95 (m, 1H), 6.80 (t, J=7.8 Hz, 2H), 4.61 (d, J=1.8 Hz, 2H), 3.58 (d, J=2.4 Hz, 3H), 3.56-3.50 (m, 1H), 3.18-3.08 (m, 1H), 2.22-1.41 (m, 7H).

### tert-butyl 2-(bromomethyl)-6-(tert-butyl)benzoate

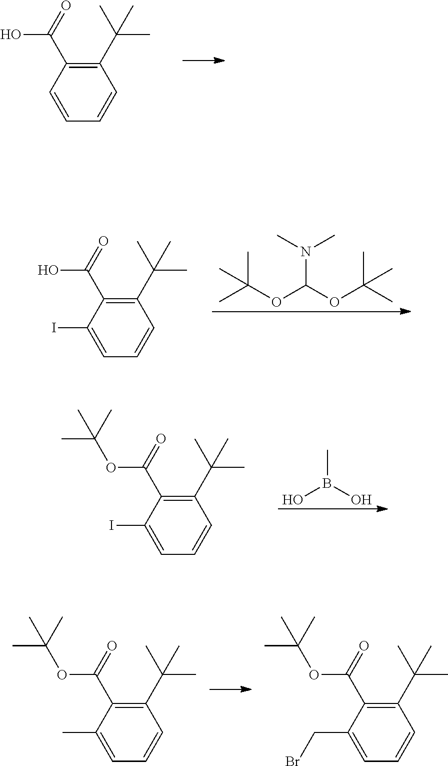

Step 1: 2-(tert-butyl)-6-iodobenzoic acid: To a solution of 2-(tert-butyl)benzoic acid (300 mg, 1.68 mmol) in DMF (5 mL) was added NIS (454 mg, 2.02 mmol) and Pd(OAc)₂ (75 mg, 0.336 mmol). The reaction was stirred at 100° C. for 3 h. The mixture was diluted with EtOAc (20 mL) and washed with water (20 mL×2), dried over Na₂SO₄, filtered and concentrated. The residue was purified by column chromatography on silica gel (eluent: DCM:MeOH=100:1 to 40:1) to afford 2-(tert-butyl)-6-iodobenzoic acid (300 mg, 58.7%) as a oranger oil. ¹H NMR (400 MHz, DMSO-d₆) δ 13.50 (s, 1H), 7.75 (dd, J=7.6, 1.0 Hz, 1H), 7.50 (dd, J=8.2, 1.2 Hz, 1H), 7.07 (t, J=8.0 Hz, 1H), 1.34 (s, 9H).

Step 2: tert-butyl 2-(tert-butyl)-6-iodobenzoate: To a solution of 2-(tert-butyl)-6-iodobenzoic acid (300 mg, 0.987 mmol) in THF (3 mL) was added 1,1-di-tert-butoxy-N,N-dimethylmethanamine (1 g, 4.93 mmol). The mixture reaction was stirred at 75° C. overnight. The mixture was diluted with EtOAc (20 mL) and washed with water (20 mL×2), dried over Na₂SO₄, filtered and concentrated to afford 2-(tert-butyl)-6-iodobenzoic acid (100 mg, 28%) as a colourless oil. ¹H NMR (400 MHz, DMSO-d₆) δ 7.76 (dd, J=7.8, 1.2 Hz, 1H), 7.51 (dd, J=8.2, 1.2 Hz, 1H), 7.08 (t, J=8.0 Hz, 1H), 1.60 (s, 9H), 1.34 (s, 9H).

Step 3: tert-butyl 2-(tert-butyl)-6-methylbenzoate: To a solution of tert-butyl 2-(tert-butyl)-6-iodobenzoate (370 mg, 1.03 mmol) in 1,4-dioxane (4 mL) was added methylboronic acid (185 mg, 3.08 mmol), Pd(dppf)Cl₂ (37 mg, 0.052 mmol) and K₂CO₃ (285 mg, 2.06 mmol). The mixture was stirred at 90° C. for 2 h under N₂ atmosphere. The mixture was diluted with EtOAc (20 mL) and washed with water (50 mL×2), dried over Na₂SO₄, filtered and concentrated. The residue was purified by pre-TLC (Pet ether:EtOAc=10:1) to afford tert-butyl 2-(tert-butyl)-6-methylbenzoate (220 mg, 86.6%) as a colourless oil. ¹H NMR (400 MHz, DMSO-d₆) δ 7.29 (d, J=8.0 Hz, 1H), 7.23 (d, J=7.2 Hz, 1H), 7.08 (d, J=7.2 Hz, 1H), 2.24 (s, 3H), 1.56 (s, 9H), 1.35 (s, 9H).

Step 4: tert-butyl 2-(bromomethyl)-6-(tert-butyl)benzoate To a solution of tert-butyl 2-(tert-butyl)-6-methylbenzoate (130 mg, 0.524 mmol) in CCl₄ (1 mL) was added NBS (47 mg, 0.262 mmol) and AIBN (7 mg, 0.0524 mmol). The mixture was stirred at 80° C. for 3 h. The mixture was diluted with EtOAc (20 mL) and washed with water (20 mL×2), dried over Na₂SO₄, filtered and concentrated. The residue was purified by pre-TLC (Pet ether:EtOAc=10:1) to afford tert-butyl 2-(bromomethyl)-6-(tert-butyl)benzoate (78 mg, 45.8%) as a colourless oil. ¹H NMR (400 MHz, Methanol-d₄) δ 7.50-7.47 (m, 1H), 7.35-7.31 (m, 2H), 4.54 (s, 2H), 1.67 (s, 9H), 1.42 (s, 9H).

### tert-butyl 2-(bromomethyl)benzoate

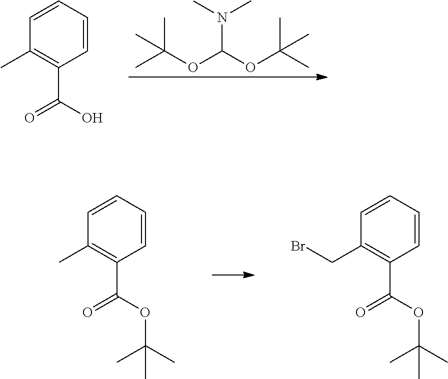

Step 1: tert-butyl 2-methylbenzoate To a solution of 2-methylbenzoic acid (1 g, 7.34 mmol) in THF (5 mL) was added 1,1-di-tert-butoxy-N,N-dimethylmethanamine (5.9 g, 29.38 mmol) and stirred at 75° C. overnight. The mixture was diluted with water (30 mL) and extracted with EtOAc (60 mL). The combined organic layer was dried over Na₂SO₄, filtered and concentrated. The residue was purified by column chromatography on silica gel (eluent: Pet. Ether/EtOAc=50:1) to afford tert-butyl 2-methylbenzoate (400 mg, 28% yield) as a colorless oil. ¹H NMR (400 MHz, DMSO-d₆) δ 7.71 (dd, J=8.2, 1.4 Hz, 1H), 7.42 (td, J=7.4, 1.4 Hz, 1H), 7.30-7.24 (m, 2H), 2.48 (s, 3H), 1.54 (s, 9H).

Step 2: tert-butyl 2-(bromomethyl)benzoate To a solution of tert-butyl 2-methylbenzoate (400 mg, 2.08 mmol) in CCl₄ (5 mL) was added NBS (371 mg, 2.08 mmol) and AIBN (34 mg, 0.21 mmol). The mixture was stirred at 80° C. for 4 h. The mixture was diluted with water (30 mL) and extracted with EtOAc (30 mL). The organic layer was dried over Na₂SO₄, filtered and concentrated. The residue was purified by pre-TLC (eluent: Pet. Ether:EtOAc=70:1) to afford tert-butyl 2-(bromomethyl)benzoate (320 mg, 56.9% yield) as a yellow oil. ¹H NMR (400 MHz, DMSO-d₆) δ 7.79 (d, J=7.6 Hz, 1H), 7.55 (dd, J=4.6, 1.4 Hz, 2H), 7.46-7.41 (m, 1H), 4.99 (s, 2H), 1.58 (s, 9H).

### tert-butyl 2-((4,4-difluorocyclohexyl)oxy)-6-hydroxybenzoate

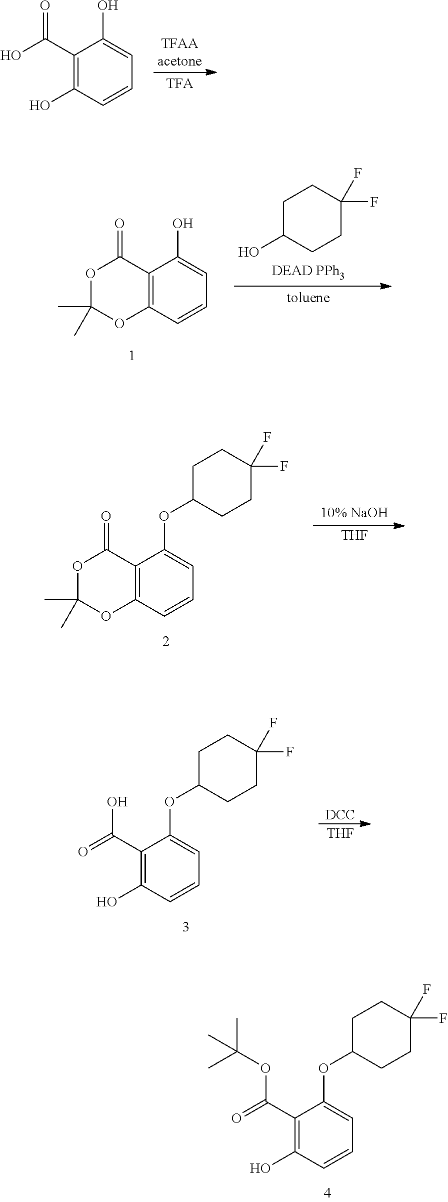

Step 1: 5-hydroxy-2,2-dimethyl-4H-benzo[d][1,3]dioxin-4-one To a solution of 2,6-dihydroxybenzoic acid (5.0 g, 32.4 mmol) in TFA (45 mL) and acetone (15 mL) was added TFAA (13.5 mL). The reaction mixture was heated to reflux for 4 h. The solvent was removed under vacuum, the residue was diluted with sodium bicarbonate aquatic solution (50 mL) and extracted with EtOAc (50 mL×3). The combined organic layer was washed with brine, dried over Na₂SO₄ and concentrated to afford crude 5-hydroxy-2,2-dimethyl-4H-benzo[d][1,3]dioxin-4-one (6.7 g, quant.) which was used directly in the next step. ¹H NMR (400 MHz, Chloroform-d) δ 7.41 (t, J=8.2 Hz, 1H), 6.63 (dd, J=8.6, 1.0 Hz, 1H), 6.44 (dd, J=8.2, 1.0 Hz, 1H), 1.75 (s, 6H).

Step 2: 5-((4,4-difluorocyclohexyl)oxy)-2,2-dimethyl-4H-benzo[d][1,3]dioxin-4-one To a solution of 5-hydroxy-2,2-dimethyl-4H-benzo[d][1,3]dioxin-4-one (2.0 g, 10.3 mmol), 4,4-difluorocyclohexan-1-ol (1.68 g, 12.3 mmol), DEAD (3.59 g, 20.6 mmol) and PPh₃ (5.4 g, 20.6 mmol) in toluene (20 mL). The reaction mixture was stirred at room temperature for 4 h. The mixture was filtered and concentrated and purified by column chromatography on silica gel (eluent: Pet.ether:EtOAc=30:1) to afford 5-((4,4-difluorocyclohexyl)oxy)-2,2-dimethyl-4H-benzo[d][1,3]dioxin-4-one (2.6 g, 81%) as a yellow solid. LCMS m/z=313.2 [M+H]⁺; ¹H NMR (400 MHz, DMSO-d₆) δ 7.56 (t, J=8.4 Hz, 1H), 6.92 (d, J=8.4 Hz, 1H), 6.63 (d, J=8.2 Hz, 1H), 4.85 (s, 1H), 2.27-2.06 (m, 2H), 2.04-1.86 (m, 4H), 1.86-1.74 (m, 2H), 1.64 (s, 6H).

Step 3: 2-((4,4-difluorocyclohexyl)oxy)-6-hydroxybenzoic acid: To a solution of 5-((4,4-difluorocyclohexyl)oxy)-2,2-dimethyl-4H-benzo[d][1,3]dioxin-4-one (2.6 g, 8.3 mmol) in a mixed solvent of THF/H₂O (20 mL/5 mL) was added NaOH (1.6 g, 41.6 mmol). The reaction mixture was stirred at 80° C. for 4 h. The reaction was diluted with water (30 mL) and extracted with EtOAc (30 mL). The aqueous layer was collected and acidified with 1 M HCl to pH˜6 and extracted with EtOAc (50 mL×3). The combined organic layers were washed with brine, dried over Na₂SO₄, filtered and concentrated to afford 2-((4,4-difluorocyclohexyl)oxy)-6-hydroxybenzoic acid (2.17 g, 95%) as a yellow solid. LCMS m/z=273.0 [M+H]⁺; ¹H NMR (400 MHz, Chloroform-d) δ 12.13 (s, 1H), 7.40 (t, J=8.4 Hz, 1H), 6.73 (d, J=8.6 Hz, 1H), 6.50 (d, J=8.2 Hz, 1H), 4.83-4.70 (m, 1H), 2.26-1.91 (m, 9H).

Step 4: tert-butyl 2-((4,4-difluorocyclohexyl)oxy)-6-hydroxybenzoate To a solution of 2-((4,4-difluorocyclohexyl)oxy)-6-hydroxybenzoic acid (920 mg, 3.38 mmol) in tert-Butanol/THF (27 mL/9 mL) were added DMAP (82.5 mg, 0.67 mmol) and DCC (1.05 g, 5.07 mmol). The reaction mixture was stirred at room temperature overnight. The mixture was diluted with H₂O (10 mL) and extracted with EtOAc (10 mL×2). The combined organic layers were washed with brine, dried over Na₂SO₄, filtered and concentrated. The residue was purified by column chromatography on silica gel (eluent: Pet.ether:EtOAc=80:1) to afford tert-butyl 2-((4,4-difluorocyclohexyl)oxy)-6-hydroxybenzoate (290 mg, 26%) as a colorless oil. LCMS m z=327.2 [M−H]⁻; ¹H NMR (400 MHz, Chloroform-d) δ 11.14 (s, 1H), 6.56 (d, J=8.4 Hz, 1H), 6.40 (d, J=8.4 Hz, 1H), 4.55-4.43 (m, 1H), 2.28-2.11 (m, 3H), 2.03-1.87 (m, 7H), 1.61 (s, 9H).

### Tert-butyl 2-((4,4-difluorocyclohexyl)methyl)-6-hydroxybenzoate

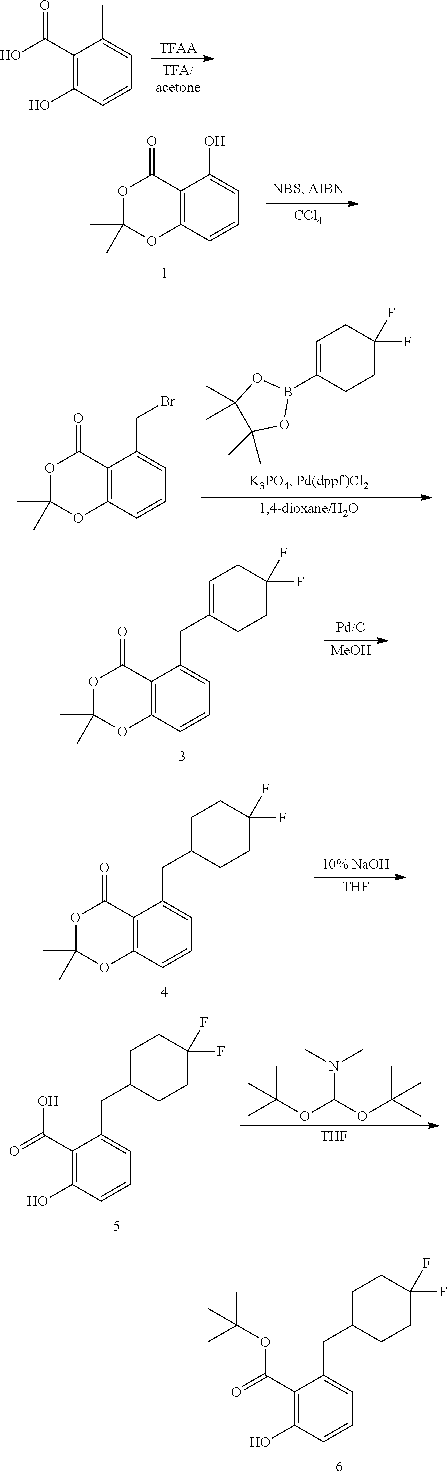

Step 1: 2,2,5-trimethyl-4H-benzo[d][1,3]dioxin-4-one To a solution of 2-hydroxy-6-methylbenzoic acid (4 g, 26 mmol) in TFA/acetone (33 mL/11 mL) was added TFAA (16 g, 78 mmol). The resulting mixture was stirred at 50° C. for 5 h. The mixture was concentrated directly, diluted with water (50 mL) and extracted with EtOAc (50 mL×3). The combined organic layers were washed with brine, dried over Na₂SO₄, filtered and concentrated to afford 2,2,5-trimethyl-4H-benzo[d][1,3]dioxin-4-one (3.5 g, 70%) as a yellow solid. LCMS: m/z=192.4 [M+H]⁺; ¹H NMR (400 MHz, CDCl₃) δ 7.41-7.35 (m, 1H), 6.93-6.90 (m, 1H), 6.80 (d, J=8.2 Hz, 1H), 2.68 (s, 3H), 1.70 (s, 6H).

Step 2: 5-(bromomethyl)-2,2-dimethyl-4H-benzo[d][1,3]dioxin-4-one To a solution of 2,2,5-trimethyl-4H-benzo[d][1,3]dioxin-4-one (3.5 g, 18.2 mmol) in CCl₄ (40 mL) was added NBS (4 g, 22.8 mmol) and AIBN (598 mg, 3.6 mmol). The resulting mixture was stirred at 80° C. for 4 h. The mixture was diluted with water (50 mL) and extracted with EtOAc (50 mL×3). The combined organic layers were washed with brine, dried over Na₂SO₄, filtered and concentrated. The crude was purified by column chromatography on silica gel (eluent: Pet ether:EtOAc=90:1) to afford 5-(bromomethyl)-2,2-dimethyl-4H-benzo[d][1,3]dioxin-4-one (1.8 g, 37%) as a white solid. LCMS: m/z=270.3 [M+H]⁺; ¹H NMR (400 MHz, CDCl₃) δ 7.48 (t, J=8.0 Hz, 1H), 7.16 (d, J=7.6 Hz, 1H), 6.94 (d, J=8.4 Hz, 1H), 5.05 (s, 2H), 1.73 (s, 6H).

Step 3: 5-((4,4-difluorocyclohex-1-en-1-yl)methyl)-2,2-dimethyl-4H-benzo[d][1,3]dioxin-4-one To a solution of 5-(bromomethyl)-2,2-dimethyl-4H-benzo[d][1,3]dioxin-4-one (900 mg, 3.3 mmol) in 1,4-dioxane/H₂O (9 mL/3 mL) was added 2-(4,4-difluorocyclohex-1-en-1-yl)-4,4,5,5-tetramethyl-1,3,2-dioxaborolane (967 mg, 4.0 mmol), K₃PO₄ (1.4 g, 6.6 mmol) and Pd(dppf)Cl₂ (239 mg, 0.3 mmol). The resulting mixture was stirred at 80° C. for 5 h under N₂ atmosphere. The mixture was diluted with water (30 mL) and extracted with EtOAc (30 mL×3). The combined organic layers were washed with brine, dried over Na₂SO₄, filtered and concentrated. The crude was purified by column chromatography on silica gel (eluent: Pet ether:EtOAc=70:1) to afford 5-((4,4-difluorocyclohex-1-en-1-yl)methyl)-2,2-dimethyl-4H-benzo[d][1,3]dioxin-4-one (417 mg, 42%) as a colorless oil. ¹H NMR (400 MHz, CD₃OD) δ 7.52 (t, J=7.8 Hz, 1H), 7.00 (d, J=7.6 Hz, 1H), 6.92 (d, J=8.2 Hz, 1H), 5.09 (s, 1H), 3.81 (s, 2H), 2.43 (t, J=14.4 Hz, 2H), 2.27-2.21 (m, 2H), 2.05-1.94 (m, 2H), 1.68 (s, 6H).

Step 4: 5-((4,4-difluorocyclohexyl)methyl)-2,2-dimethyl-4H-benzo[d][1,3]dioxin-4-one To a solution of 5-((4,4-difluorocyclohex-1-en-1-yl)methyl)-2,2-dimethyl-4H-benzo[d][1,3]dioxin-4-one (417 mg, 1.4 mmol) in MeOH (5 mL) was added Pd/C (42 mg). The resulting mixture was stirred at 45° C. for 8 h. The mixture was filtered and concentrated to afford 5-((4,4-difluorocyclohexyl)methyl)-2,2-dimethyl-4H-benzo[d][1,3]dioxin-4-one (389 mg, 93%) as a colorless oil. ¹H NMR (400 MHz, CD₃OD) δ 7.50 (t, J=7.8 Hz, 1H), 6.98 (d, J=7.6 Hz, 1H), 6.90 (d, J=9.0 Hz, 1H), 3.04 (d, J=6.6 Hz, 2H), 2.04-1.95 (m, 2H), 1.75-1.67 (m, 10H), 1.37-1.28 (m, 3H).

Step 5: 2-((4,4-difluorocyclohexyl)methyl)-6-hydroxybenzoic acid To a solution of 5-((4,4-difluorocyclohexyl)methyl)-2,2-dimethyl-4H-benzo[d][1,3]dioxin-4-one (389 mg, 1.3 mmol) in THF (4 mL) was added 10% NaOH (1 mL). The resulting mixture was stirred at 80° C. for 6 h. The mixture was acidified with 1 M HCl to pH˜3 and extracted with EtOAc (30 mL×3). The combined organic layers were washed with brine, dried over Na₂SO₄, filtered and concentrated to afford 2-((4,4-difluorocyclohexyl)methyl)-6-hydroxybenzoic acid (325 mg, 96%) as a white solid. LCMS: m/z=269.2 [M−H]⁻; ¹H NMR (400 MHz, CD₃OD) δ 7.24 (t, J=7.8 Hz, 1H), 6.76 (d, J=8.2 Hz, 1H), 6.68 (d, J=7.6 Hz, 1H), 2.87 (d, J=6.6 Hz, 2H), 2.00-1.96 (m, 1H), 1.74-1.65 (m, 4H), 1.37-1.21 (m, 4H).

Step 6: tert-butyl 2-((4,4-difluorocyclohexyl)methyl)-6-hydroxybenzoate To a solution of 2-((4,4-difluorocyclohexyl)methyl)-6-hydroxybenzoic acid (325 mg, 1.2 mmol) in THF (4 mL) was added 1,1-di-tert-butoxy-N,N-dimethylmethanamine (976 mg, 4.8 mmol). The resulting mixture was stirred at 80° C. for 4 h. The mixture was concentrated directly and purified by prep-TLC (eluent: Pet ether:EtOAc=10:1) to afford tert-butyl 2-((4,4-difluorocyclohexyl)methyl)-6-hydroxybenzoate (172 mg, 44%) as a colorless oil. ¹H NMR (400 MHz, CD₃OD) δ 7.20-7.15 (m, 1H), 6.74-6.64 (m, 2H), 2.68 (d, J=6.8 Hz, 2H), 2.04-1.95 (m, 2H), 1.72-1.65 (m, 4H), 1.61 (s, 9H), 1.34-1.23 (m, 3H).

### 1-(3,5-difluorobenzyl)-1H-pyrazole-4-carboxylic acid

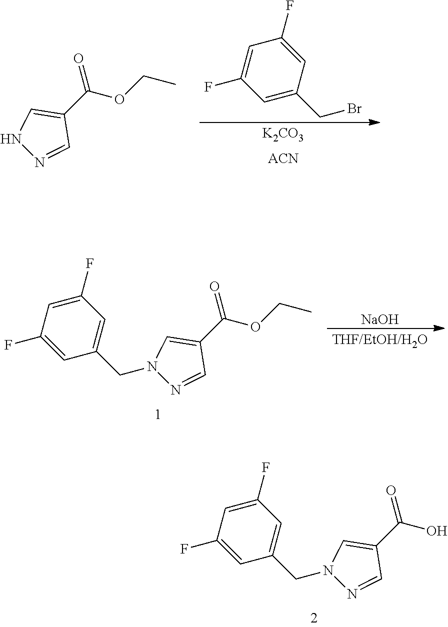

Step 1: ethyl 1-(3,5-difluorobenzyl)-1H-pyrazole-4-carboxylate To a solution of ethyl 1H-pyrazole-4-carboxylate (1.0 g, 4.83 mmol) in ACN (10 mL) was added K₂CO₃ (2.0 g, 14.49 mmol) and 1-(bromomethyl)-3,5-difluorobenzene (812 mg, 5.8 mmol). The mixture was stirred at room temperature overnight. The mixture was diluted with water (100 ml) and extracted with EtOAc (60 mL×3). The organic layers were washed with brine, dried over Na₂SO₄, filtered and concentrated to afford ethyl 1-(3,5-difluorobenzyl)-1H-pyrazole-4-carboxylate (1.23 g, 95%) as white solid. LCMS: m/z=267.2 [M+H]⁺; ¹HNMR (400 MHz, DMSO-d₆) δ 8.51 (s, 1H), 7.90 (s, 1H), 7.24-7.15 (m, 1H), 7.02-6.94 (m, 2H), 5.40 (s, 2H), 4.27-4.17 (m, 2H), 1.28-1.24 (m, 3H).

Step 2: 1-(3,5-difluorobenzyl)-1H-pyrazole-4-carboxylic acid To a solution of ethyl 1-(3,5-difluorobenzyl)-1H-pyrazole-4-carboxylate (1.0 g, 3.75 mmol) in a mixture of THF (10 mL), EtOH (2.5 mL) and water (2.5 mL) was added NaOH (450 mg, 18.75 mmol). The reaction was stirred at 50° C. for 14 h. The mixture was diluted with water (50 mL) and extracted with EtOAc (30 mL). The aqueous layer was collected and acidified with 1 M HCl to pH˜2 and extracted with EtOAc (50 mL×2). The combined organic layers were washed with brine, dried over Na₂SO₄, filtered and concentrated to afford 1-(3,5-difluorobenzyl)-1H-pyrazole-4-carboxylic acid (830 mg, 93%) as a white solid. LCMS: m/z=239.1 [M+H]⁺; ¹HNMR (400 MHz, DMSO-d₆) δ 12.38 (s, 1H), 8.42 (s, 1H), 7.85 (s, 1H), 7.25-7.15 (m, 1H), 7.02-6.94 (m, 2H), 5.39 (s, 2H).

### tert-butyl 2-(bromomethyl)-6-(4,4-difluorocyclohexyl)-3-fluorobenzoate

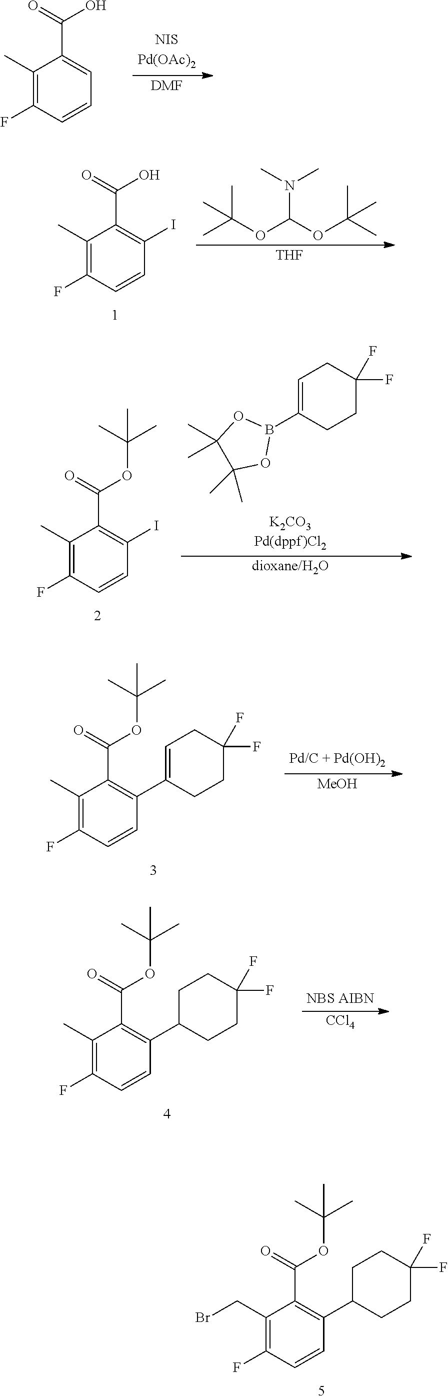

Step 1: 3-fluoro-6-iodo-2-methylbenzoic acid To a solution of 3-fluoro-2-methylbenzoic acid (1.0 g, 6.49 mmol) and NIS (1.61 g, 7.14 mmol) in DMF (10 mL) was added Pd(OAc)₂ (145 mg, 0.65 mmol). The reaction mixture was stirred at 100° C. for 2 h. The mixture was diluted with water (30 mL) and extracted with EtOAc (30 mL×2). The combined organic layers were washed with water and brine, dried over Na₂SO₄, filtered and concentrated. The crude was purified by RP-column (60% ACN in water) to afford 3-fluoro-6-iodo-2-methylbenzoic acid (1.8 g, 98%) as yellow solid. ¹H NMR (400 MHz, Chloroform-d) δ 7.65 (dd, J=8.6, 4.8 Hz, 1H), 6.86 (t, J=8.8 Hz, 1H), 2.36 (t, J=2.0 Hz, 3H).

Step 2: tert-butyl 3-fluoro-6-iodo-2-methylbenzoate To a solution of 3-fluoro-6-iodo-2-methylbenzoic acid (500 mg, 1.79 mmol) in THF (5 mL) was added 1,1-di-tert-butoxy-N,N-dimethylmethanamine (1.45 g, 7.14 mmol). The mixture was heated to reflux for 3 h. The solvent was removed and the residue was purified by column chromatography on silica gel (eluent: Pet.ether:EtOAc=100:1) to afford tert-butyl 3-fluoro-6-iodo-2-methylbenzoate (300 mg, 50%) as a white solid. LCMS m/z=279.25 [M-99]*; ¹H NMR (400 MHz, Chloroform-d) δ 7.58 (dd, J=8.6, 4.8 Hz, 1H), 6.78 (t, J=8.8 Hz, 1H), 2.26 (d, J=2.2 Hz, 3H), 1.63 (s, 9H).

Step 3: tert-butyl 4,4′,4′-trifluoro-3-methyl-2′,3′,4′,5′-tetrahydro-[1,1′-biphenyl]-2-carboxylate To a solution of tert-butyl 3-fluoro-6-iodo-2-methylbenzoate (300 mg, 0.89 mmol), K₂CO₃ (246 mg, 1.78 mmol) and Pd(dppf)Cl₂ (65 mg, 0.89 mmol) in the mixture of 1,4-dioxane (3 mL) and H₂O (1 mL) was added 2-(4,4-difluorocyclohex-1-en-1-yl)-4,4,5,5-tetramethyl-1,3,2-dioxaborolane (261 mg, 1.07 mmol). The reaction mixture was stirred at 100° C. under N₂ atmosphere for 2 h. The mixture was diluted with water (10 mL) and extracted with EtOAc (10 mL×2). The combined organic layers were washed with brine, dried over Na₂SO₄, filtered and concentrated. The residue was purified by prep-TLC (eluent: Pet. Ether:EtOAc=20:1) to afford tert-butyl 4,4′,4′-trifluoro-3-methyl-2′,3′,4′,5′-tetrahydro-[1,1′-biphenyl]-2-carboxylate (220 mg, 75%) as a white solid. ¹H NMR (400 MHz, Chloroform-d) δ 7.00-6.96 (m, 2H), 5.49 (qt, J=3.8, 2.3 Hz, 1H), 2.68-2.53 (m, 4H), 2.25 (d, J=2.2 Hz, 3H), 2.21-2.06 (m, 2H), 1.54 (s, 9H).

Step 4: tert-butyl 6-(4,4-difluorocyclohexyl)-3-fluoro-2-methylbenzoate To a solution of tert-butyl 4,4′,4′-trifluoro-3-methyl-2′,3′,4′,5′-tetrahydro-[1,1′-biphenyl]-2-carboxylate (230 mg, 0.7 mmol) in MeOH (3 mL) was added 10% Pd/C (92 mg) and Pd(OH)₂ (92 mg). The reaction mixture was stirred under H₂ atmosphere for 4 h. The mixture was filtered through celite and concentrated to afford tert-butyl 6-(4,4-difluorocyclohexyl)-3-fluoro-2-methylbenzoate (170 mg, 73%) as a colorless oil which was used directly in the next step. ¹H NMR (400 MHz, Chloroform-d) δ 7.11-6.96 (m, 2H), 2.69-2.59 (m, 1H), 2.23 (d, J=2.1 Hz, 3H), 2.15-2.25 (m, 2H), 1.93 (d, J=10.5 Hz, 2H), 1.88-1.71 (m, 4H), 1.61 (s, 9H).

Step 5: tert-butyl 2-(bromomethyl)-6-(4,4-difluorocyclohexyl)-3-fluorobenzoate: To a solution of tert-butyl 2-(bromomethyl)-6-(4,4-difluorocyclohexyl)-3-fluorobenzoate (150 mg, 0.46 mmol) in CCl₄ (2 mL) was added NBS (98 mg, 0.55 mmol) and AIBN (15 mg, 0.91 mmol). The reaction mixture was stirred at 80° C. for Ih. The mixture was diluted with water (10 mL) and extracted with DCM (10 mL×2). The combined organic layers were washed with brine, dried over Na₂SO₄, filtered and concentrated. The residue was purified by prep-TLC (eluent: Pet. ether:EtOAc=20:1) to afford tert-butyl 2-(bromomethyl)-6-(4,4-difluorocyclohexyl)-3-fluorobenzoate (120 mg, 48%) as a yellow solid. ¹H NMR (400 MHz, Chloroform-d) δ 7.23 (dd, J=8.8, 5.2 Hz, 1H), 7.07 (s, 1H), 4.53 (d, J=1.5 Hz, 2H), 2.68 (d, J=11.8 Hz, 1H), 2.23-2.18 (m, 2H), 1.97-1.90 (m, 2H), 1.86-1.72 (m, 4H), 1.66 (s, 9H).

### 7-bromo-4,5-difluorobenzo[d]thiazole

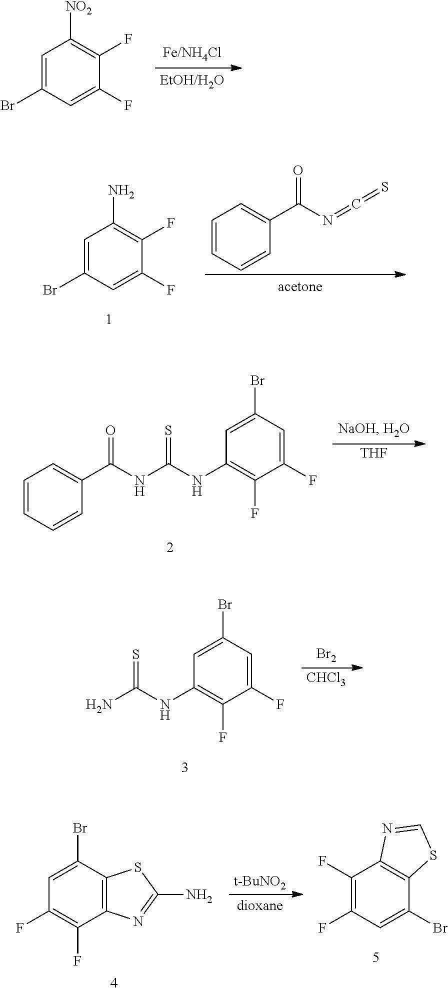

Step 1: 5-bromo-2,3-difluoroaniline To a solution of 5-bromo-1,2-difluoro-3-nitrobenzene (3.8 g, 15.93 mmol) and NH₄Cl (2.6 g, 47.78 mmol) in EtOH/H₂O (40 mL/10 mL) was added Fe (2.7 g, 47.78 mmol). The reaction mixture was stirred at reflux for 1 h. The mixture was diluted with water (30 mL) and extracted with EtOAc (30 mL×2). The combined organic layers were washed with brine, dried over Na₂SO₄, filtered and concentrated. The crude was purified by column chromatography on silica gel (eluent: Pet.ether:EtOAc=30:1) to afford 5-bromo-2,3-difluoroaniline (7.68 g, 85%) as yellow oil. LCMS m/z=208.1 [M+H]⁺; ¹H NMR (400 MHz, Chloroform-d) δ 6.81-6.55 (m, 2H), 3.90 (s, 2H).

Step 2: N-((5-bromo-2,3-difluorophenyl)carbamothioyl)benzamide a solution of 5-bromo-2,3-difluoroaniline (270 mg, 1.3 mmol) in acetone (5 mL) was added benzoyl isothiocyanate (211 mg, 1.3 mmol). The reaction mixture was stirred at room temperature for 30 minutes. The mixture was filtered and concentrated to afford N-((5-bromo-2,3-difluorophenyl)carbamothioyl)benzamide (200 mg, 30%) as yellow solid. LCMS m/z=369.0 [M−H]⁻; ¹H NMR (400 MHz, Methanol-d₄) δ 8.38 (d, J=5.6 Hz, 1H), 7.98 (d, J=7.6 Hz, 2H), 7.66 (d, J=7.4 Hz, 1H), 7.56 (t, J=7.6 Hz, 2H), 7.47 (s, 1H).

Step 3: 1-(5-bromo-2,3-difluorophenyl)thiourea To a solution of N-((5-bromo-2,3-difluorophenyl)carbamothioyl)benzamide (2.0 g, 5.4 mmol) in THE (20 mL) was added 10% NaOH (10 mL). The reaction mixture was stirred at 80° C. for 3 h. The mixture was diluted with water (30 mL) and extracted with EtOAc (30 mL×3). The combined organic layers were washed with brine, dried over Na₂SO₄, filtered and concentrated to afford 1-(5-bromo-2,3-difluorophenyl)thiourea (2.4 g, quant.) as a yellow solid. LCMS m/z=266.0 [M+H]⁺; ¹H NMR (400 MHz, DMSO-d₆) δ 9.61 (s, 1H), 7.87 (dt, J=6.0, 2.2 Hz, 1H), 7.61 (ddd, J=9.4, 6.4, 2.4 Hz, 1H).

Step 4: 7-bromo-4,5-difluorobenzo[d]thiazol-2-amine To a solution of 1-(5-bromo-2,3-difluorophenyl)thiourea (1.4 g, 5.24 mmol) in Chloroform (20 mL) was added Br₂ (1.3 g, 7.9 mmol). The reaction mixture was heated to reflux for 4 h. The mixture was filtered and the solid was collected. The solid was treated with Na₂CO₃ solution (20 mL) and extracted with EtOAc (20 mL×2). The combined organic layers were washed with brine, dried over Na₂SO₄, filtered and concentrated to afford 7-bromo-4,5-difluorobenzo[d]thiazol-2-amine (1.03 g, 74%) as yellow solid. LCMS m/z=265.0 [M+H]⁺; ¹H NMR (400 MHz, DMSO-d₆) δ 8.15 (d, J=2.8 Hz, 2H), 7.43-7.35 (m, 1H).

Step 5: 7-bromo-4,5-difluorobenzo[d]thiazole To a solution of 7-bromo-4,5-difluorobenzo[d]thiazol-2-amine (300 mg, 1.13 mmol) in 1,4-dioxane (3 mL) was added t-BuNO₂ (233 mg, 2.3 mmol). The reaction mixture was stirred at 85° C. for 2 h. The mixture was diluted with water (5 mL) and extracted with EtOAc (10 mL×2). The combined organic layers were washed with brine, dried over Na₂SO₄, filtered and concentrated. The crude was purified by column chromatography on silica gel (eluent: Pet.ether:EtOAc=60:1) to afford 7-bromo-4,5-difluorobenzo[d]thiazole (130 mg, 45%) as yellow solid. H NMR (400 MHz, DMSO-d₆) δ 9.61 (s, 1H), 8.07 (dd, J=10.2, 6.2 Hz, 1H).

### tert-butyl 2-(bromomethyl)-6-(5-(trifluoromethyl)thiophen-3-yl)benzoate

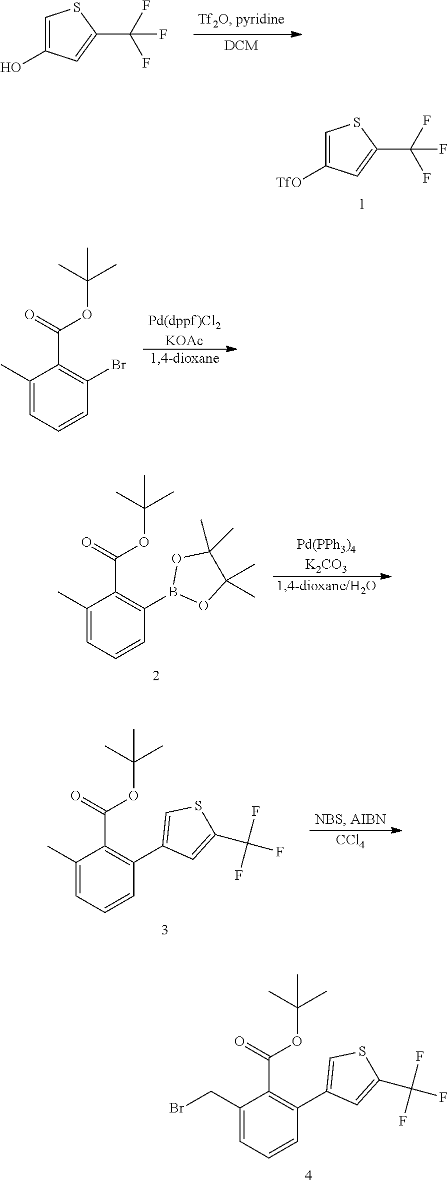

Step 1: 5-(trifluoromethyl)thiophen-3-yl trifluoromethanesulfonate To a solution of 5-(trifluoromethyl)thiophen-3-ol (90 mg, 0.54 mmol) in DCM (1 mL) were added pyridine (85 mg, 1.07 mmol) and Trifluoromethanesulfonic anhydride (302 mg, 1.07 mmol) and the reaction mixture was stirred at 0° C. for 3 h. Water was added and the aqueous extracted with EtOAc. The combined organic layers were washed with water, brine and dried over Na₂SO₄, concentrated to afford 5-(trifluoromethyl)thiophen-3-yl trifluoromethanesulfonate (100 mg, 61%) as a green oil. ¹H NMR (400 MHz, DMSO-d₆) δ 8.29 (d, J=1.8 Hz, 1H), 8.10-8.08 (m, 1H).

Step 2: tert-butyl 2-methyl-6-(4,4,5,5-tetramethyl-1,3,2-dioxaborolan-2-yl)benzoate To a solution of tert-butyl 2-bromo-6-methylbenzoate (300 mg, 1.11 mmol) in 1,4-dioxane (3 mL) was added 4,4,4′,4′,5,5,5′,5′-octamethyl-2,2′-bi(1,3,2-dioxaborolane) (562 mg, 2.22 mmol), potassium acetate (327 mg, 3.33 mmol) and Pd(dppf)Cl₂ (80 mg, 0.11 mmol). The reaction was then stirred at 80° C. overnight. The reaction was cooled to room temperature, diluted with water (30 mL) and extracted with EtOAc (30 mL). The combined organic layers were washed with brine, dried over Na₂SO₄, filtered and concentrated. The mixture was purified by prep-TLC (eluent: Pet ether:EtOAc=15:1) to afford tert-butyl 2-methyl-6-(4,4,5,5-tetramethyl-1,3,2-dioxaborolan-2-yl)benzoate (150 mg, 42%) as a colorless oil. ¹H NMR (400 MHz, CD3OD) δ 7.51-7.46 (m, 1H), 7.36-7.25 (m, 2H), 2.39 (s, 3H), 1.63 (s, 9H), 1.34 (s, 12H).

Step 3: tert-butyl 2-methyl-6-(5-(trifluoromethyl)thiophen-3-yl)benzoate To a solution of 5-(trifluoromethyl)thiophen-3-yl trifluoromethanesulfonate (91 mg, 0.30 mmol), tert-butyl 2-methyl-6-(4,4,5,5-tetramethyl-1,3,2-dioxaborolan-2-yl)benzoate (106 mg, 0.33 mmol) and K₂CO₃ (124 mg, 0.90 mmol) in a mixture solvent of 1,4-dioxane/H₂O (3 mL/0.5 mL) under a N₂ atmosphere was added Pd(PPh₃)₄ (35 mg, 0.03 mmol). The reaction mixture was stirred at 100° C. for 2 h. The mixture was diluted with water (20 mL) and extracted with EtOAc (30 mL×3). The combined organic layers were washed with brine, dried over Na₂SO₄, filtered and concentrated. The residue was purified by prep-TLC (eluent: Pet ether:EtOAc=100:3) to afford tert-butyl 2-methyl-6-(5-(trifluoromethyl)thiophen-3-yl)benzoate (50 mg, 48% yield) as a yellow oil. ¹H NMR (400 MHz, CD3OD) δ 7.65-7.64 (m, 1H), 7.57-7.54 (m, 1H), 7.38-7.33 (m, 1H), 7.29-7.21 (m, 2H), 4.57 (s, 3H), 2.37 (s, 3H), 1.39 (s, 9H).

Step 4: tert-butyl 2-(bromomethyl)-6-(5-(trifluoromethyl)thiophen-3-yl)benzoate: To a solution of tert-butyl 2-methyl-6-(5-(trifluoromethyl)thiophen-3-yl)benzoate (50 mg, 0.42 mmol) in CCl₄ (0.5 mL) was added NBS (31 mg, 0.18 mmol) and AIBN (5 mg, 0.03 mmol). The reaction mixture was stirred at 80° C. overnight. The mixture was diluted with water (50 mL) and extracted with DCM (30 mL×2). The combined organic layers were washed with brine, dried over Na₂SO₄, fitered and concentreated. The residue was purified by prep-TLC (eluent: Pet ether:EtOAc=20:1) to afford tert-butyl 2-(bromomethyl)-6-(5-(trifluoromethyl)thiophen-3-yl)benzoate (38 mg, 62%) as yellow oil. ¹H NMR (400 MHz, CD3OD) δ 7.69-7.66 (m, 1H), 7.58-7.55 (m, 1H), 7.53-7.45 (m, 2H), 7.40-7.37 (m, 1H), 4.69 (s, 2H), 1.41-1.39 (m, 9H).

### tert-butyl 2-bromo-4-fluoro-6-methylbenzoate

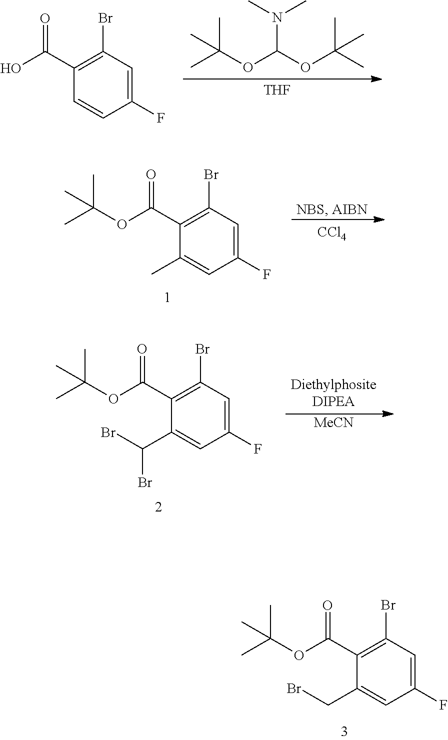

Step 1: tert-butyl 2-bromo-4-fluoro-6-methylbenzoate To a solution of 2-bromo-4-fluorobenzoic acid (900 mg, 3.8 mmol) in THF (45 mL) was heated at reflux was added 1,1-di-tert-butoxy-N,N-dimethylmethanamine (3.6 mL) for 3 h. The mixture was concentrated. The residue was purified by column chromatography on silica gel (eluent: Pet. Ether:EtOAc=50:1) to afford tert-butyl 2-bromo-4-fluoro-6-methylbenzoate (630 mg, 70%) as a yellow oil. ¹HNMR (400 MHz, CD3OD) δ 7.24 (dd, J=8.3, 2.4 Hz, 1H), 7.03 (dd, J=9.3, 2.4 Hz, 1H), 2.35 (s, 3H), 1.61 (s, 9H).

Step 2: tert-butyl 2-bromo-6-(dibromomethyl)-4-fluorobenzoate To a solution of tert-butyl 2-bromo-4-fluoro-6-methylbenzoate (180 mg, 0.62 mmol) in CCl₄ (5 mL) was added NBS (220 mg, 1.24 mmol) and AIBN (19.7 mg, 0.12 mmol). The reaction mixture was stirred at reflux for 2.5 h. The mixture was diluted with water (20 mL) and extracted with EtOAc (20 mL×3). The combined organic layers were washed with brine, dried over Na₂SO₄, fitered and concentreated to afford tert-butyl 2-bromo-6-(dibromomethyl)-4-fluorobenzoate (330 mg, quant.) as a white solid.

Step 3: tert-butyl 2-bromo-4-fluoro-6-methylbenzoate To a solution of tert-butyl 2-bromo-6-(dibromomethyl)-4-fluorobenzoate (330 mg, 0.74 mmol) in MeCN (5 mL) was added DIPEA (143 mg, 1.11 mmol) and diethylphosphite (153 mg, 1.11 mmol). The resulting mixture was stirred at room temperature for 3.5 hours. The mixture was diluted with water (30 mL) and extracted with EtOAc (50 mL×2). The combined organic layer was washed with brine, dried over Na₂SO₄, filtered and concentrated. The residue was purified by column chromatography on silica gel (eluent: Pet. Ether:EtOAc=50:1) to afford tert-butyl 2-bromo-4-fluoro-6-methylbenzoate (180 mg, 66%) as a colorless oil. 1HNMR (400 MHz, CD3OD) δ 7.43 (dd, J=8.1, 2.3 Hz, 1H), 7.29 (dd, J=9.0, 2.5 Hz, 1H), 4.57 (s, 2H), 1.64 (s, 9H).

### tert-butyl 6-bromo-2-(bromomethyl)-3-methoxybenzoate

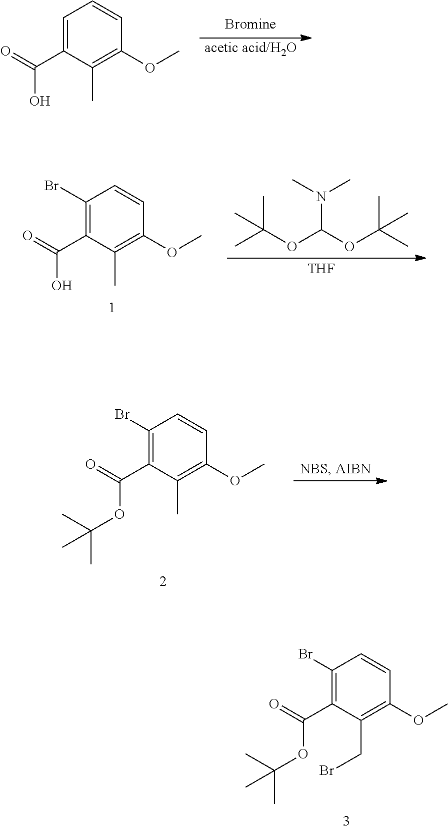

Step 1: 6-bromo-3-methoxy-2-methylbenzoic acid To a solution of 3-methoxy-2-methylbenzoic acid (1.9 g, 11.4 mmol) in H₂O/acetic acid (10 mL/10 mL) was added Bromine (2 g, 12.5 mmol). The resulting mixture was stirred at 60° C. for 3 h. The mixture was diluted with water (50 mL) and extracted with EtOAc (30 mL×3). The combined organic layers were washed with brine, dried over Na₂SO₄, filtered and concentrated to afford 6-bromo-3-methoxy-2-methylbenzoic acid (3.2 g, quant.) as a yellow oil. LCMS: m/z=241.6 [M+H]⁺; ¹H NMR (400 MHz, CDCl₃) δ 7.37 (d, J=8.8 Hz, 1H), 6.76 (d, J=8.8 Hz, 1H), 3.83 (s, 3H), 2.27 (s, 3H).

Step 2: tert-butyl 6-bromo-3-methoxy-2-methylbenzoate To a solution of 6-bromo-3-methoxy-2-methylbenzoic acid (1.2 g, 4.9 mmol) in THF (13 mL) was added 1,1-di-tert-butoxy-N,N-dimethylmethanamine (4 g, 19.6 mmol). The resulting mixture was stirred at reflux for 3 h. The solvent was removed under vacuum. The crude was purified by column (Pet ether:EtOAc=95:1) to afford tert-butyl 6-bromo-3-methoxy-2-methylbenzoate (850 mg, 58%) as a colorless oil. LCMS: m/z=246.3 [M-56]⁺; ¹H NMR (400 MHz, CDCl₃) δ 7.32 (d, J=8.8 Hz, 1H), 6.73-6.68 (m, 1H), 3.80 (s, 3H), 2.19 (s, 3H), 1.62 (s, 9H).

Step 3: tert-butyl 6-bromo-2-(bromomethyl)-3-methoxybenzoate To a solution of tert-butyl 6-bromo-3-methoxy-2-methylbenzoate (300 mg, 1.0 mmol) in CCl₄ (4 mL) was added NBS (222 mg, 1.3 mmol) and AIBN (33 mg, 0.2 mmol). The resulting mixture was stirred at 80° C. for 4 h. The mixture was diluted with water (30 mL) and extracted with EtOAc (30 mL×3). The combined organic layers were washed with brine, dried over Na₂SO₄, filtered and concentrated to afford tert-butyl 6-bromo-2-(bromomethyl)-3-methoxybenzoate (538 mg, quant.) as a yellow oil. ¹H NMR (400 MHz, CDCl₃) δ 7.45 (d, J=8.8 Hz, 1H), 6.78 (d, J=8.8 Hz, 1H), 4.54 (s, 2H), 3.89 (s, 3H), 1.66 (s, 9H).

### 1-(2-(difluoromethoxy)-4-fluorobenzyl)-1H-pyrazole-4-carboxylic acid

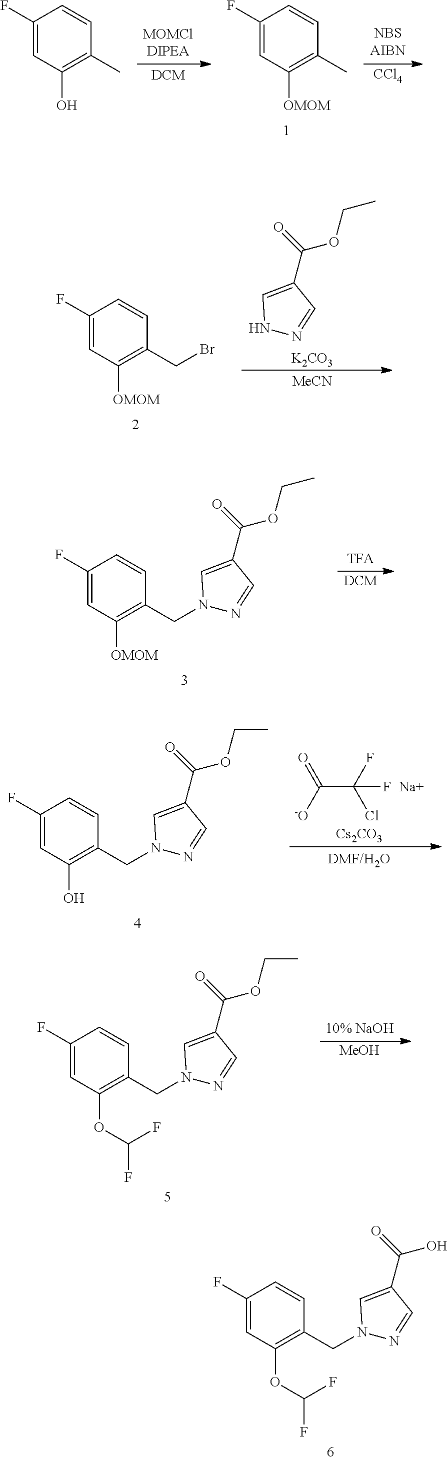

Step 1: 4-fluoro-2-(methoxymethoxy)-1-methylbenzene To a solution of 5-fluoro-2-methylphenol (1 g, 8 mmol) in DCM (10 mL) was added DIPEA (3.1 g, 24 mmol) and MOMCl (760 mg, 9.5 mmol). The mixture was stirred at reflux for 14 h. The mixture was diluted with water (60 mL) and extracted with DCM (50 mL×2). The combined organic layers were washed with brine, dried over Na₂SO₄, filtered and concentrated. The crude was purified by column chromatography on silica gel (eluent: Pet. Ether:EtOAc=30:1) to afford 4-fluoro-2-(methoxymethoxy)-1-methylbenzene (830 mg, 61%) a colorless oil. ¹H NMR (400 MHz, DMSO) δ 7.20-7.14 (m, 1H), 6.91-6.85 (m, 1H), 6.75-6.68 (m, 1H), 5.23 (s, 2H), 3.38 (s, 3H), 2.14 (s, 3H).

Step 2: 1-(bromomethyl)-4-fluoro-2-(methoxymethoxy)benzene To a solution of 4-fluoro-2-(methoxymethoxy)-1-methylbenzene (80 mg, 0.47 mmol) in CCl₄ (2 mL) was added NBS (100 mg, 0.56 mmol) and AIBN (8.2 mg, 0.05 mmol). The mixture was stirred at reflux for 8 h. The mixture was diluted with water (20 mL) and extracted with DCM (20 mL×2). The combined organic layers were washed with brine, dried over Na₂SO₄, filtered and concentrated. The residue was purified by prep-TLC (eluent: Pet. Ether:EtOAc=30:1) to afford ethyl 1-(4-fluoro-2-(methoxymethoxy)benzyl)-1H-pyrazole-4-carboxylate (56 mg, 48%) as a colorless oil. ¹H NMR (400 MHz, DMSO) δ 7.52-7.45 (m, 1H), 7.02-6.95 (m, 1H), 6.85-6.79 (m, 1H), 5.33 (s, 2H), 4.66 (s, 2H), 3.43 (s, 3H).

Step 3: ethyl 1-(4-fluoro-2-(methoxymethoxy)benzyl)-1H-pyrazole-4-carboxylate: To a solution of 1-(bromomethyl)-4-fluoro-2-(methoxymethoxy)benzene (56 mg, 0.23 mmol) in MeCN (1 mL) was added ethyl 1H-pyrazole-4-carboxylate (37.8 mg, 0.27 mmol) and K₂CO₃ (95 mg, 0.69 mmol). The mixture was stirred at room temperature for 4 h. The mixture was diluted with water (10 mL) and extracted with EtOAc (10 mL×2). The combined organic layers were washed with brine, dried over Na₂SO₄, filtered and concentrated. The residue was purified by prep-TLC (eluent: Pet. Ether:EtOAc=5:1) to afford ethyl 1-(4-fluoro-2-(methoxymethoxy)benzyl)-1H-pyrazole-4-carboxylate (20 mg, 29%) as a colorless oil. ¹H NMR (400 MHz, DMSO) δ 8.30 (s, 1H), 7.84 (s, 1H), 7.19-7.13 (m, 1H), 7.00-6.94 (m, 1H), 6.85-6.78 (m, 1H), 5.32 (s, 2H), 5.27 (s, 2H), 4.23-4.16 (m, 2H), 1.27-1.21 (m, 3H).

Step 4: ethyl 1-(4-fluoro-2-hydroxybenzyl)-1H-pyrazole-4-carboxylate To a solution of ethyl 1-(4-fluoro-2-(methoxymethoxy)benzyl)-1H-pyrazole-4-carboxylate (840 mg, 2.7 mmol) in DCM (16 mL) was added TFA (6 mL). The mixture was stirred at room temperature for overnight. The mixture was diluted with water (20 mL) and extracted with DCM (30 mL×2). The combined organic layers were washed with brine, dried over Na₂SO₄, filtered and concentrated to afford ethyl 1-(4-fluoro-2-hydroxybenzyl)-1H-pyrazole-4-carboxylate (770 mg, 97%) as a yellow oil. ¹H NMR (400 MHz, CD₃OD) δ 8.09 (s, 1H), 7.85 (s, 1H), 7.20-7.12 (m, 1H), 6.62-6.49 (m, 2H), 5.28 (s, 2H), 4.29-4.24 (m, 2H), 1.34-1.31 (m, 3H).

Step 5: ethyl 1-(2-(difluoromethoxy)-4-fluorobenzyl)-1H-pyrazole-4-carboxylate: To a solution of ethyl 1-(4-fluoro-2-hydroxybenzyl)-1H-pyrazole-4-carboxylate (770 g, 2.8 mmol) in DMF/H₂O (18 mL/2 mL) was added sodium 2-chloro-2,2-difluoroacetate (1 g, 7 mmol) and Cs₂CO₃ (1.8 g, 5.6 mmol). The mixture was stirred at 100° C. for 14 h. The mixture was diluted with water (60 mL) and extracted with EtOAc (50 mL). The combined organic layers were washed with brine, dried over Na₂SO₄, filtered and concentrated. The crude was purified by column chromatography on silica gel (eluent: Pet. Ether:EtOAc=30:1) to afford ethyl 1-(2-(difluoromethoxy)-4-fluorobenzyl)-1H-pyrazole-4-carboxylate (180 mg, 20%) as a colorless oil. ¹H NMR (400 MHz, CD₃OD) δ 8.17 (s, 1H), 7.88 (s, 1H), 7.31-7.26 (m, 1H), 7.13-6.75 (m, 3H), 5.39 (s, 2H), 4.31-4.24 (m, 2H), 1.36-1.31 (m, 3H).

Step 6: 1-(2-(difluoromethoxy)-4-fluorobenzyl)-1H-pyrazole-4-carboxylic acid: To a solution of ethyl 1-(2-(difluoromethoxy)-4-fluorobenzyl)-1H-pyrazole-4-carboxylate (180 mg, 0.57 mmol) in MeOH (3 mL) was added 10% NaOH (2 mL). The mixture was stirred at 50° C. for 2 h. The mixture was diluted with water (20 mL) and adjusted the pH to 2 with 1 N HCl, then extracted with EtOAc (20 mL×2). The combined organic layers were washed with brine, dried over Na₂SO₄, filtered and concentrated to afford 1-(2-(difluoromethoxy)-4-fluorobenzyl)-1H-pyrazole-4-carboxylic acid (140 mg, 85%) as a yellow solid. LCMS m/z=287.1 [M+H]; ¹H NMR (400 MHz, DMSO) δ 12.35 (s, 1H), 8.27 (s, 1H), 7.81 (s, 1H), 7.50-7.08 (m, 4H), 5.36 (s, 2H).

### 3-(4,4-difluorocyclohexyl)-1H-pyrazole

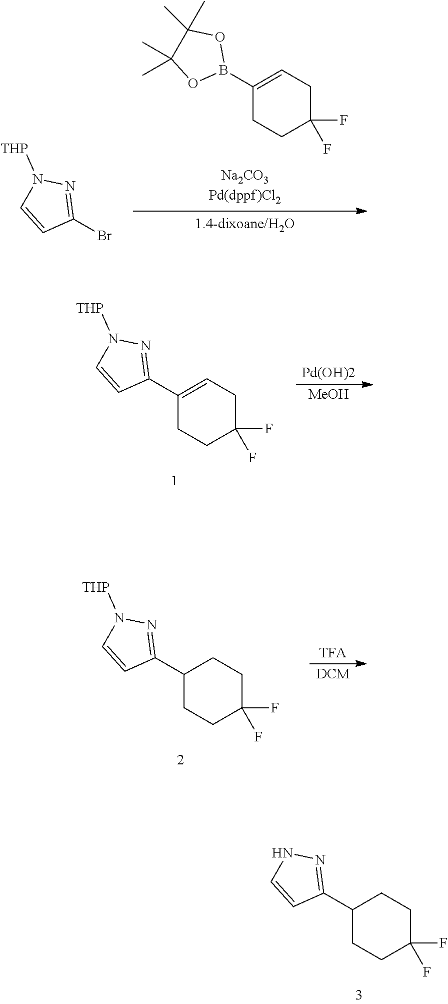

Step 1: 3-(4,4-difluorocyclohex-1-en-1-yl)-1-(tetrahydro-2H-pyran-2-yl)-1H-pyrazole: To a solution of 3-bromo-1-(tetrahydro-2H-pyran-2-yl)-1H-pyrazole (2 g, 8.65 mmol), in dioxane (20 mL)/H2O (5 mL), 2-(4,4-difluorocyclohex-1-en-1-yl)-4,4,5,5-tetramethyl-1,3,2-dioxaborolane (2.53 g, 10.3 mmol), Na2CO3 (2.75 g, 25.96 mmol) and Pd(pddf)Cl2 (628 mg, 0.86 mmol). The reaction mixture was stirred at 110° C. for 3 h. The mixture was diluted with water (150 mL) and extracted with EtOAc (100 mL×4). The combined organic layers were washed with brine, dried over Na₂SO₄, filtered and concentrated. The crude product was purified by column chromatography on silica gel (eluent: Pet. Ether:EtOAc=50:1 to 20:1) to afford 3-(4,4-difluorocyclohex-1-en-1-yl)-1-(tetrahydro-2H-pyran-2-yl)-1H-pyrazole (1.5 g, 65%) as a colorless oil. LCMS: m/z=269.1[M+H]⁺; ¹HNMR (400 MHz, DMSO-d₆) δ 7.81 (d, J=2.5 Hz, 1H), 6.48 (d, J=2.5 Hz, 1H), 6.15-6.08 (m, 1H), 5.38-5.30 (m, 1H), 3.96-3.87 (m, 1H), 3.66-3.55 (m, 1H), 2.75-2.60 (m, 4H), 2.16-2.00 (m, 3H), 1.98-1.83 (m, 2H), 1.74-1.59 (m, 1H), 1.55-1.47 (m, 2H).

Step 2: 3-(4,4-difluorocyclohexyl)-1-(tetrahydro-2H-pyran-2-yl)-1H-pyrazole: To a solution of 3-(4,4-difluorocyclohex-1-en-1-yl)-1-(tetrahydro-2H-pyran-2-yl)-1H-pyrazole (1.5 g, 5.59 mmol) in MeOH (15 mL) was added 40% Pd(OH)2 (600 mg). The reaction mixture was stirred under a H2 atmosphere for 4 h. The mixture was filtered and concentrated to afford 3-(4,4-difluorocyclohexyl)-1-(tetrahydro-2H-pyran-2-yl)-1H-pyrazole (1.2 g, 80%) which was used directly in the next step without further purification. LCMS: m/z=271.2[M+H]⁺; ¹HNMR (400 MHz, Chloroform-d) δ 7.51 (d, J=2.4 Hz, 1H), 6.12 (d, 1H), 5.40-5.26 (m, 1H), 4.18-3.98 (m, 1H), 3.77-3.61 (m, 1H), 2.89-2.70 (m, 1H), 2.25-1.97 (m, 6H), 1.95-1.50 (m, 7H), 1.33-1.21 (m, 1H).

Step 3: 3-(4,4-difluorocyclohexyl)-1H-pyrazole To a solution of 3-(4,4-difluorocyclohexyl)-1-(tetrahydro-2H-pyran-2-yl)-1H-pyrazole (1.2 g, 4.4 mmol) in DCM (12 mL) was added TFA (12 mL). The reaction mixture was stirred at room temperature for 4 h. The solvent was diluted with saturated sodium carbonate solution (20 mL) and extracted with EtOAc ((30 mL×3), The organic layers were washed with brine, dried over Na₂SO₄, filtered and concentrated to afford 3-(4,4-difluorocyclohexyl)-1H-pyrazole (135 mg, 100%) as yellow oil. LCMS: m/z=187.1[M+H]⁺;

### tert-butyl 3-(bromomethyl)-2′,4′,6′-trifluoro-[1,1′-biphenyl]-2-carboxylate

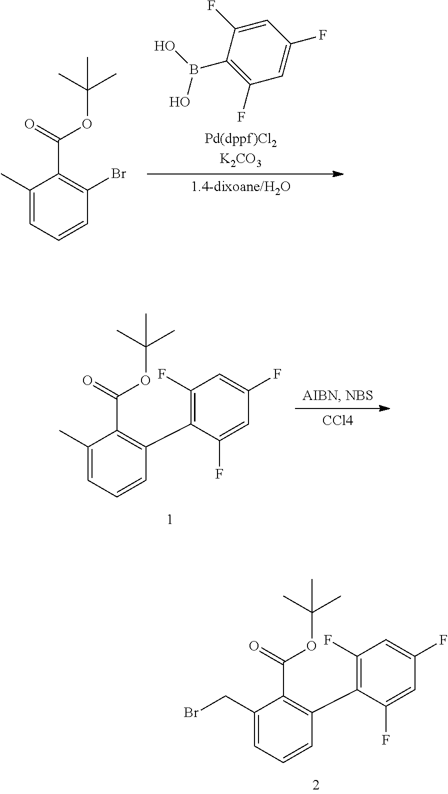

Step 1: tert-butyl 2′,4′,6′-trifluoro-3-methyl-[1,1′-biphenyl]-2-carboxylate To a solution of tert-butyl 2-bromo-6-methylbenzoate (300 mg, 1.1 mmol) in DMF (3 mL) was added (2,4,6-trifluorophenyl)boronic acid (214 mg, 1.22 mmol), CsF (252 mg, 1.66 mmol) and Ag₂O (192 mg, 0.83 mmol). The mixture was stirred at 100° C. under N₂ atmosphere for 24 h. The mixture was diluted with water (40 ml) and extracted with EtOAc (30 mL×3). The organic layers were washed with brine, dired over Na₂SO₄, filtered and concentrated, The residue obtained was purified by prep-TLC (Per ether/EtOAc=20/1) to afford tert-butyl 2′,4′,6′-trifluoro-3-methyl-[1,1′-biphenyl]-2-carboxylate (180 mg, 50%) as white solid. ¹HNMR (400 MHz, Methanol-d₄) δ 7.48-7.33 (m, 2H), 7.14 (d, J=7.5 Hz, 1H), 7.01-6.92 (m, 2H), 2.44 (s, 3H), 1.33 (s, 9H).

Step 2: tert-butyl 3-(bromomethyl)-2′,4′,6′-trifluoro-[1,1′-biphenyl]-2-carboxylate To a solution of tert-butyl 2′,4′,6′-trifluoro-3-methyl-[1,1′-biphenyl]-2-carboxylate (150 mg, 0.46 mmol) in a mixture of CCl₄ (2 mL) was added AIBN (15 mg, 0.09 mmol), NBS (82 mg, 0.46 mmol). The reaction was stirred at 85° C. for 6 h then diluted with water (50 mL) and extracted with EtOAc (30 mL). The combined organic layers were washed with brine, dried over Na₂SO₄, filtered and concentrated to afford tert-butyl 3-(bromomethyl)-2′,4′,6′-trifluoro-[1,1′-biphenyl]-2-carboxylate (100 mg, 53%) as a white solid. ¹HNMR (400 MHz, Methanol-d₄) δ 7.63-7.51 (m, 2H), 7.34-7.30 (m, 1H), 7.05-6.94 (m, 2H), 4.82 (s, 2H), 1.36 (s, 9H).

### ethyl 2-(2-(bromomethyl)-6-(4,4-difluorocyclohexyl)phenyl)cyclopropane-1-carboxylate

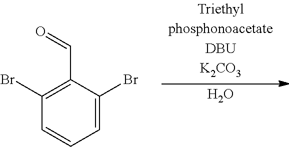

Step 1: ethyl (E)-3-(2,6-dibromophenyl)acrylate To a solution of 2,6-dibromobenzaldehyde (10.0 g, 38.2 mmol) in H₂O (6 mL) was added triethyl phosphonoacetate (8.6 g, 38.2 mmol), DBU (174 mg, 1.15 mmol) and K₂CO₃ (10.6 g, 76.4 mmol). The resulting mixture was stirred at room temperature for 3 h. The mixture was diluted with H₂O (100 mL) and extracted with EtOAc (100 mL×3). The combined organic layers were washed with brine, dried over Na₂SO₄, filtered and concentrated. The residue obtained was purified by column chromatography on silica gel (eluent: Pet ether/EtOAc=60/1) to afford ethyl (E)-3-(2,6-dibromophenyl)acrylate (12.4 mg, 99%) as a yellow oil. ¹H NMR (400 MHz, CDCl₃) δ 7.65 (d, J=16.2 Hz, 1H), 7.57 (d, J=8.0 Hz, 2H), 7.01 (t, J=8.0 Hz, 1H), 6.39 (d, J=16.2 Hz, 1H), 4.29 (q, J=7.2 Hz, 2H), 1.35 (t, J=7.2 Hz, 3H).

Step 2: ethyl 2-(2,6-dibromophenyl)cyclopropane-1-carboxylate To a solution of ethyl (E)-3-(2,6-dibromophenyl)acrylate (5.0 g, 15.1 mmol) in THF (50 mL) was added tetrafluoro(methyldiphenyl-14-sulfanyl)-15-borane (5.6 g, 19.6 mmol) and NaHMDS (17 mL, 2.0 M, 33.2 mmol) at 0° C. under N₂. The resulting mixture was stirred at room temperature over night. The mixture was diluted with water (50 mL) and extracted with EtOAc (50 mL×3). The combined organic layers were washed with brine, dried over Na₂SO₄, filtered and concentrated. The residue obtained was purified by column chromatography on silica gel (eluent: PE/EtOAc=60/1) to afford ethyl 2-(2,6-dibromophenyl)cyclopropane-1-carboxylate (2.4 g, 46%) as a yellow oil. ¹H NMR (400 MHz, CDCl₃) δ 7.52 (d, J=8.0 Hz, 2H), 6.95 (t, J=8.0 Hz, 1H), 4.30-4.17 (m, 2H), 2.48-2.36 (m, 1H), 1.95-1.85 (m, 1H), 1.85-1.75 (m, 1H), 1.43-1.36 (m, 1H), 1.30 (t, J=7.1 Hz, 3H).

Step 3: ethyl 2-(3-bromo-4′,4′-difluoro-2′,3′,4′,5′-tetrahydro-[1,1′-biphenyl]-2-yl)cyclopropane-1-carboxylate To a solution of ethyl 2-(2,6-dibromophenyl)cyclopropane-1-carboxylate (1.0 g, 2.9 mmol) in 1,4-dioxane/H₂O (12 mL/3 mL) was added 2-(4,4-difluorocyclohex-1-en-1-yl)-4,4,5,5-tetramethyl-1,3,2-dioxaborolane (706 mg, 2.9 mmol), Pd(dppf)Cl₂ (218 mg, 0.3 mmol) and K₃PO₄ (1.85 g, 8.7 mmol). The resulting mixture was stirred at 100° C. for 3 h. The mixture was diluted with water (50 mL) and extracted with EtOAc (50 mL×3). The combined organic layers were washed with brine, dried over Na₂SO₄, filtered and concentrated. The residue obtained was purified by column chromatography on silica gel (eluent: Pet ether/EtOAc=80/1) to afford ethyl 2-(3-bromo-4′,4′-difluoro-2′,3′,4′,5′-tetrahydro-[1,1′-biphenyl]-2-yl)cyclopropane-1-carboxylate (450 mg, 41%) as a yellow oil. ¹H NMR (400 MHz, CDCl₃) δ 7.53-7.47 (m, 1H), 7.09-7.00 (m, 2H), 5.60-5.41 (m, 1H), 4.20 (q, J=7.1 Hz, 2H), 2.76-2.53 (m, 4H), 2.49-2.42 (m, 1H), 2.23-2.12 (m, 2H), 1.75-1.70 (m, 1H), 1.63 (d, J=4.4 Hz, 1H), 1.29 (t, J=7.2 Hz, 3H), 1.24-1.19 (m, 1H).

Step 4: ethyl 2-(4′,4′-difluoro-3-methyl-2′,3′,4′,5′-tetrahydro-[1,1′-biphenyl]-2-yl)cyclopropane-1-carboxylate To a solution of ethyl 2-(3-bromo-4′,4′-difluoro-2′,3′,4′,5′-tetrahydro-[1,1′-biphenyl]-2-yl)cyclopropane-1-carboxylate (450 mg, 1.3 mmol) in 1,4-dioxane (5 mL) was added 2,4,6-trimethyl-1,3,5,2,4,6-trioxatriborinane (1.5 mL, 3.5 M, 2.6 mmol), Pd(dppf)Cl₂ (94 mg, 0.13 mmol) and K₃PO₄ (828 mg, 3.9 mmol). The resulting mixture was stirred at 100° C. for 3 h. The mixture was diluted with water (30 mL) and extracted with EtOAc (30 mL×3). The combined organic layers were washed with brine, dried over Na₂SO₄, filtered and concentrated. The residue obtained was purified by column chromatography on silica gel (eluent: Pet erther/EtOAc=80/1) to afford ethyl 2-(4′,4′-difluoro-3-methyl-2′,3′,4′,5′-tetrahydro-[1,1′-biphenyl]-2-yl)cyclopropane-1-carboxylate (320 mg, 77%) as a yellow oil. ¹H NMR (400 MHz, CDCl₃) δ 7.15-7.04 (m, 2H), 6.95 (dd, J=6.1, 1.2 Hz, 1H), 5.58-5.40 (m, 1H), 4.23-4.17 (m, 2H), 2.78-2.54 (m, 4H), 2.39 (s, 3H), 2.25-2.10 (m, 2H), 1.72-1.63 (m, 1H), 1.60-1.55 (m, 2H), 1.31 (t, J=7.2 Hz, 3H), 1.17-1.07 (m, 1H).

Step 5: ethyl 2-(2-(4,4-difluorocyclohexyl)-6-methylphenyl)cyclopropane-1-carboxylate To a solution of ethyl 2-(4′,4′-difluoro-3-methyl-2′,3′,4′,5′-tetrahydro-[1,1′-biphenyl]-2-yl)cyclopropane-1-carboxylate (320 mg, 1.0 mmol) in MeOH (8 mL) was added Pd/C (128 mg) and Pd(OH)₂ (128 mg). The resulting mixture was stirred at 45° C. for 3 h. The mixture was filtered, washed with DCM/MeOH=10/1 and concentrated to afford ethyl 2-(2-(4,4-difluorocyclohexyl)-6-methylphenyl)cyclopropane-1-carboxylate (280 mg, 87%) as a colorless oil. ¹H NMR (400 MHz, CDCl₃) δ 7.19-7.10 (m, 1H), 7.12-7.05 (m, 1H), 7.06-6.99 (m, 1H), 4.33-4.18 (m, 2H), 3.35-3.20 (m, 1H), 2.40 (s, 3H), 2.34-2.29 (m, 1H), 2.27-2.19 (m, 2H), 1.89-1.71 (m, 8H), 1.34 (t, J=7.1 Hz, 3H), 1.15-1.07 (m, 1H).

Step 6: ethyl 2-(2-(bromomethyl)-6-(4,4-difluorocyclohexyl)phenyl)cyclopropane-1-carboxylate To a solution of ethyl 2-(2-(4,4-difluorocyclohexyl)-6-methylphenyl)cyclopropane-1-carboxylate (280 mg, 0.87 mmol) in CCl₄ (6 mL) was added NBS (186 mg, 1.04 mmol) and AIBN (29 mg, 0.17 mmol). The resulting mixture was stirred at 80° C. for 3 h. The mixture was diluted with water (20 mL) and extracted with EtOAc (20 mL×3). The combined organic layers were washed with brine, dried over Na₂SO₄, filtered and concentrated to afford ethyl 2-(2-(bromomethyl)-6-(4,4-difluorocyclohexyl)phenyl)cyclopropane-1-carboxylate (240 mg, 69%) as a colorless oil. ¹H NMR (400 MHz, CDCl₃) δ 7.26-7.22 (m, 2H), 7.22-7.19 (m, 1H), 4.81-4.66 (m, 2H), 4.34-4.22 (m, 2H), 3.34-3.20 (m, 1H), 2.52-2.40 (m, 1H), 2.26-2.17 (m, 2H), 1.93-1.73 (m, 8H), 1.35 (t, J=7.2 Hz, 4H).

### (2-bromo-6-(bromomethyl)phenyl)(methyl)sulfane

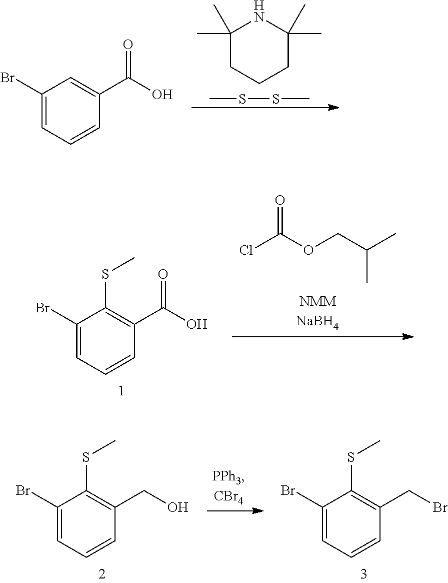

Step 1: 3-bromo-2-(methylthio)benzoic acid To a solution of 2,2,6,6-tetramethylpiperidine (15.5 g, 110 mmol) in THE (20 mL) was added n-BuLi (7 g, 110 mmol) at −78° C. The mixture was stirred at −78° C. for 0.5 h. Then to a solution of 3-bromobenzoic acid (10 g, 50 mmol) in THF (60 mL) was added LTMP (110 mmol) at −78° C. The resulting mixture was stirred at −78° C. for 1 h. Then added 1,2-dimethyldisulfane (9.4 g, 100 mmol), stirred at room temperature over night. The mixture was acidified with 1 M HCl to PH˜3 and extracted with EtOAc (50 mL×3). The combined organic layers were washed with brine, dried over Na₂SO₄, filtered, concentrated and purified by column chromatography on silica gel (eluent: DCM/MeOH=75:1) to afford 3-bromo-2-(methylthio)benzoic acid (3.8 g, 32%) as a yellow solid. ¹HNMR (400 MHz, CD₃OD) δ 7.79-7.75 (m, 1H), 7.49-7.44 (m, 1H), 7.31-7.26 (m, 1H), 2.42 (s, 3H).

Step 2: (3-bromo-2-(methylthio)phenyl)methanol To a solution of 3-bromo-2-(methylthio)benzoic acid (3.6 g, 14.5 mmol), isobutyl carbonochloridate (2.8 g, 20.3 mmol) and NMM (1.9 g, 18.9 mmol) in THE (30 mL) was stirred at 0° C. for 0.5 h, NaBH₄ (1.7 g, 43.5 mmol) in H₂O (10 mL) was dropwise added. The resulting mixture was stirred at room temperature for 2 h. The mixture was diluted with water (30 mL×3) and extracted with EtOAc (30 mL×3). The combined organic layers were washed with brine, dried over Na₂SO₄, filtered, concentrated to afford (3-bromo-2-(methylthio)phenyl)methanol (3.8 g, quant.) as a yellow oil. ¹HNMR (400 MHz, DMSO-d₆) δ 7.62 (d, J=9.0 Hz, 1H), 7.57-7.53 (m, 1H), 7.34-7.29 (m, 1H), 4.76 (s, 2H), 2.30 (s, 3H).

Step 3: (2-bromo-6-(bromomethyl)phenyl)(methyl)sulfane To a solution of (3-bromo-2-(methylthio)phenyl)methanol (4 g, 17.2 mmol) in DCM (40 mL) was added PPh3 (5 g, 20.6 mmol) and CBr₄ (6.8 g, 20.6 mmol). The resulting mixture was stirred at room temperature for 4 h. The mixture was diluted with water (30 mL×3) and extracted with EtOAc (30 mL×3). The combined organic layers were washed with brine, dried over Na₂SO₄, filtered, concentrated and purified by column chromatography on silica gel (eluent: PE/EtOAc=60:1) to afford (2-bromo-6-(bromomethyl)phenyl)(methyl)sulfane (882 mg, 17%) as a yellow oil. ¹H NMR (400 MHz, DMSO-d₆) δ 7.75-7.70 (m, 1H), 7.62-7.58 (m, 1H), 7.34-7.28 (m, 1H), 4.96 (s, 2H), 2.42 (s, 3H).

### Example A1: Caliper Assay

Inhibition of CDK2/Cyclin E1 activity in the presence of compounds of the present disclosure was evaluated using a Caliper LabChip® EZ Reader mobility shift assay. In the assay, activated CDK2/Cyclin E1 catalyzes the phosphorylation of a fluorescently tagged peptide 5-FAM-QSPKKG-CONH2 (PerkinElmer, FL Peptide 18) which induces a difference in capillary electrophoresis mobility. The peptide substrate and product were measured, and the conversion ratio was used to determine the inhibition (as % activity and IC₅₀ values) of CDK2/Cyclin E1. Reactions contained 50 mM HEPES pH 7.5, 10 mM MgCl₂, 1 mM EDTA, 2 mM DTT, 0.01% Brij35, 0.5 mg/mL BSA, 0.1% DMSO, 2.5 nM CDK2/Cyclin E1(14-475), 100 μM ATP, and 1.5 μM fluorescent peptide substrate.

Dose titrations of inhibitors in 100% DMSO were combined with 3.25 nM CDK2/Cyclin E1(14-475) and 130 μM of ATP in reaction buffer. The mixtures were incubated for 30 minutes before the addition of fluorescent peptide substrate to initiate the kinase reaction. The final conditions were 2.5 nM CDK2/Cyclin E1(14-475), 100 μM ATP, and 1.5 μM fluorescent peptide. The reactions were stopped after 100 minutes with the addition of EDTA (6 mM final EDTA concentration). The stopped reactions were analyzed on a Caliper LabChip® EZ Reader 11. The conversion ratios were normalized to yield % activity, plotted against compound concentration, and fit to a four-parameter equation to determine the IC₅₀ for each compound.

The results of the Caliper Assay are reported in Table 27, below. Compounds with an IC₅₀ less than or equal to 0.01 μM are designated as “A”. Compounds with an IC₅₀ greater than 0.01 μM and less than or equal to 0.1 μM are designated as “B”. Compounds with an IC₅₀ greater than 0.1 μM and less than or equal to 1.0 μM are designated as “C”. Compounds with an IC₅₀ greater than 1.0 μM and less than or equal to 10.0 μM are designated as “D”. Compounds with an IC₅₀ greater than 10.0 μM are designated as “E”.

### Example A2: ADPGLO (CDK2/E1-37C)

Inhibition of CDK2/Cyclin E1 activity by the presence of small molecules was evaluated using ADP-Glo Luminescent Kinase Assay (Promega). Activated CDK2/Cyclin E1 was incubated with its substrate Histone H1 (SignalChem H10-54N) in the kinase reaction buffer (100 μM ATP, 50 mM HEPES pH 7.5, 10 mM MgCl2, 1 mM EDTA, 2 mM DTT, 0.01% Brij35, 0.5 mg/mL BSA). Luminescence was recorded with an Envision plate reader (PerkinElmer).

Dose titrations of inhibitors in 100% DMSO were combined with 0.36 nM CDK2/Cyclin E1 in reaction buffer. The mixtures were incubated for 60 minutes at 37° C. before the addition of ATP and Histone H1 substrate to initiate the kinase reaction. The final conditions were 0.18 nM CDK2/Cyclin E1, 100 μM ATP, and 1.5 M Histone H1. The reactions were incubated at 37° C. for 90 minutes before being stopped with the addition of ADP-Glo reagent. This mixture was incubated at room temperature for 60 minutes before Kinase Detection Solution is added to generate luminescence. The stopped reactions were analyzed on an Envision plate reader. The conversion ratios were normalized to yield % activity, plotted against compound concentration, and fit to a four-parameter equation to determine the IC₅₀ for each compound.

The results of the ADPGLO assay are reported in Table 27, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 M and less than or equal to 10.0 μM are designated as “C”. Compounds with an TC₅₀ greater than 10.0 μM are designated as “D”.

### Example A3: IncuCyte Kuramochi Assay

IncuCyte® assay was used to measure the effect of disclosed compounds on cell proliferation. Fluorescent microscopy images of cells were taken immediately after compound treatment and 72 hours later. Image analysis software was used to obtain cell counts as a function of compound concentration. Kuramochi cells labeled with mApple-H2B were seeded on 384-well assay-ready plates. Plates were placed in an IncuCyte® (Sartorius) and scanned at 0 and 72 hours. IncuCyte® software was used to count the number of fluorescent nuclei in each well. The fold change in cell count from 0 to 72 hours in wells treated with increasing compounds concentrations (10 pts, 1/2 log dilution, 20 M top concentration) was normalized to DMSO control wells. The normalized cell counts were fit with dose response curves and a GI50 was calculated.

The results of the Kuramochi assay are reported in Table 27, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”.

TABLE 27
Assay Results

| | | | | IncuCyte |
|---|---|---|---|---|
| | | ADPGLO | IncuCyte - | Kuramochi |
| | Caliper Assay | (CDK2/E1- | Kuramochi | RB-KO: |
| Cmpd | (CDK2/CCNE1): | 37C): IC50 | RB-WT: | IC50 - Batch |
| # | IC50 (μM) | (μM) | IC50 (μM) | Mean (μM) |
| I-1 | | C | D | |
| I-2 | | C | D | |
| I-3 | | D | D | |
| I-4 | A | B | D | |
| I-5 | B | B | C | |
| I-6 | D | D | D | |
| I-7 | E | | D | |
| I-8 | E | | D | |
| I-9 | E | | D | |
| I-10 | D | | D | |
| I-11 | E | | D | |
| I-12 | E | | D | |
| I-13 | C | | D | |
| I-14 | D | | D | |
| I-15 | E | | D | |
| I-16 | E | | C | |
| I-17 | E | | D | |
| I-18 | B | | D | |
| I-19 | E | | D | |
| I-20 | C | | D | |
| I-21 | C | | D | |
| I-22 | C | | D | |
| I-23 | D | | D | |
| I-24 | B | | C | |
| I-25 | C | | D | |
| I-26 | E | | D | |
| I-27 | E | | D | |
| I-28 | C | | D | |
| I-29 | E | | D | |
| I-30 | D | | D | |
| I-31 | C | B | C | |
| I-32 | E | | D | |
| I-33 | B | | C | |
| I-34 | B | B | C | |
| I-35 | E | | D | |
| I-36 | C | C | D | |
| I-37 | E | | D | |
| I-38 | C | D | D | |
| I-39 | C | D | D | |
| I-40 | D | | D | |
| I-41 | D | | D | |
| I-42 | D | | D | |
| I-43 | D | | D | |
| I-44 | D | | D | |
| I-45 | E | | D | |
| I-46 | E | | D | |
| I-47 | E | | D | |
| I-48 | D | | D | |
| I-49 | D | C | D | |
| I-49 | C | | D | |
| I-50 | E | D | D | |
| I-51 | | A | C | |
| I-52 | | C | D | |
| I-53 | | D | D | |
| I-54 | | A | D | |
| I-55 | | D | C | |
| I-56 | | C | D | |
| I-57 | | D | D | |
| I-58 | | E | D | |
| I-59 | | D | D | |
| I-60 | | D | D | |
| I-61 | | D | D | |
| I-62 | | E | D | |
| I-63 | | D | D | |
| I-64 | D | | D | |
| I-117 | | A | D | D |
| I-129 | | A | D | D |
| I-72 | | B | D | D |
| I-104 | | A | B | C |
| I-119 | | A | B | D |
| I-128 | | A | D | D |
| I-114 | | A | D | D |
| I-101 | | A | B | D |
| I-130 | | A | D | D |
| I-88 | | A | B | D |
| I-115 | | A | D | D |
| I-113 | | B | D | D |
| I-102 | | A | B | D |
| I-78 | | B | D | D |
| I-77 | | B | D | D |
| I-127 | | A | B | D |
| I-111 | | B | D | D |
| I-118 | | A | C | D |
| I-96 | | B | D | D |
| I-73 | | B | D | D |
| I-68 | | B | D | D |
| I-68 | | B | D | D |
| I-86 | | A | B | B |
| I-69 | | A | D | D |
| I-108 | | B | D | D |
| I-109 | | A | D | D |
| I-108 | | B | D | D |
| I-100 | | A | D | D |
| I-97 | | A | C | D |
| I-93 | | B | D | D |
| I-76 | | A | C | D |
| I-71 | | A | D | D |
| I-99 | | D | D | D |
| I-98 | | B | D | D |
| I-95 | | A | B | D |
| I-94 | | A | B | D |
| I′-11 | | D | | |
| I′-12 | | A | | |
| I′-13 | | A | | |
| I′-14 | | A | | |
| I′-15 | | B | | |
| I′-16 | | A | | |
| I′-17 | | A | | |
| I′-18 | | A | | |
| I′-19 | | A | B | D |
| I′-20 | | A | C | D |
| I′-21 | | A | D | D |
| I′-22 | | A | D | D |
| I′-23 | | A | A | D |
| I′-24 | | A | B | D |
| T′-25 | | A | D | D |
| I′-26 | | A | A | D |
| I′-27 | | A | C | D |
| I′-28 | | A | C | D |
| I′-29 | | A | D | D |
| I′-30 | | A | D | D |
| I′-31 | | A | B | C |
| I′-32 | | A | A | C |
| I′-33 | | A | D | D |
| I′-34 | | A | A | D |
| I′-35 | | A | B | D |
| I′-36 | | A | A | B |
| I′-37 | | A | A | D |
| I′-38 | | A | C | D |
| I′-39 | | B | D | D |
| I′-40 | | B | B | C |
| I′-41 | | D | C | D |
| I′-42 | | A | B | B |
| I′-43 | | A | A | C |
| I′-44 | | A | B | D |
| I′-45 | | A | B | D |
| I′-46 | | B | C | D |
| I′-47 | | A | D | D |
| I′-48 | | C | A | D |
| I′-49 | | B | D | D |
| I′-50 | | D | D | D |
| I′-51 | | A | D | D |
| I′-52 | | A | B | D |
| I′-53 | | A | B | D |
| I′-54 | | A | C | D |
| I′-55 | | A | C | D |
| I′-56 | | D | B | D |
| I′-57 | | A | C | B |
| I′-58 | | A | D | D |
| I′-59 | | D | B | D |
| I′-60 | | D | D | D |
| I′-61 | | D | D | D |
| I′-62 | | B | D | D |
| I′-63 | | A | D | D |
| I′-64 | | A | B | B |
| I′-65 | | A | D | D |
| I′-66 | | A | A | D |
| I′-67 | | A | D | D |
| I′-68 | | A | C | D |
| I′-69 | | D | C | D |
| I′-70 | | A | D | D |
| I′-71 | | A | C | D |
| I′-72 | | A | A | D |
| I′-73 | | A | A | D |
| I′-74 | | A | D | D |
| I′-75 | | A | D | D |
| I′-76 | | A | B | C |
| I′-77 | | A | C | D |
| I′-78 | | D | D | D |
| I′-79 | | A | D | D |
| I′-80 | | A | B | D |
| I′-81 | | B | D | D |
| I′-82 | | C | D | D |
| I′-83 | | B | D | D |
| I′-84 | | A | D | D |
| I′-85 | | A | A | C |
| I′-86 | | B | D | D |

1. A compound of Formula I′: 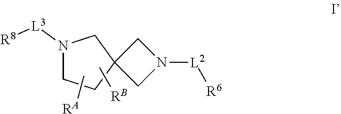
or a pharmaceutically acceptable salt thereof, wherein:
R^{A} is 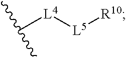
R^{B} is a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 5-12 membered saturated or partially unsaturated bicyclic carbocyclic ring that is optionally bridged, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with x instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
L⁴ is an saturated or unsaturated, straight or branched, optionally substituted bivalent C₁₋₃ hydrocarbon chain, wherein 0-3 methylene units of L⁴ are independently replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
L⁵ is a covalent bond or a saturated or unsaturated, straight or branched, optionally substituted bivalent C₁₋₂ hydrocarbon chain, wherein a 1^{st} methylene unit of L⁵ is replaced with a bivalent cyclic group selected from a 5-6 membered monocyclic heteroarylene ring (having 1-4 heteroatoms independently selected from nitrogen, oxygen, and sulfur), an 8-10 membered bicyclic aromatic carbocyclene ring, an 8-10 membered bicyclic heteroarylene ring (having 1-5 heteroatoms independently selected from nitrogen, oxygen, and sulfur), a 3-8 membered saturated or partially unsaturated monocyclic carbocyclene ring, phenyl, and a 3-8 membered saturated or partially unsaturated monocyclic heterocyclene ring (having 1-2 heteroatoms independently selected from nitrogen, oxygen, and sulfur), wherein the bivalent cyclic group is optionally substituted with y instances of R⁹; and provided that if L⁵ is a saturated or unsaturated, straight or branched, optionally substituted bivalent C₂ hydrocarbon chain wherein said 1′ methylene unit of L⁵ is replaced with said bivalent cyclic group, a 2^{nd} methylene unit of L⁵ is optionally replaced by —O—, —NR—, —S(O)₂—, —C(O)—, —S—, —C(R)₂—, —OC(O)—, —C(O)O—, —S(O)—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —C(O)N(R)S(O)₂R, —C(O)N(R)S(O)₂NR₂, an optionally substituted C₁₋₆ aliphatic group, an optionally substituted C₁₋₆ aliphatic-Cy group, or Cy;
R¹⁰ is hydrogen, halogen, an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring that is optionally bridged bicyclic or spirocyclic, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with z instances of R⁹;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 3-7 membered saturated, partially unsaturated, or heteroaryl ring (having 0-3 heteroatoms, in addition to the nitrogen, independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
x is 0, 1, 2, 3, 4, 5, 6, 7, or 8;
y is 0, 1, 2, 3, 4, 5, 6, 7, or 8; and
z is 0, 1, 2, 3, 4, 5, 6, 7, or 8.

2. (canceled)

3. The compound of claim 1, wherein R^{A} is a substituent of Table 1.

4. The compound of claim 1, wherein R^{B} is hydrogen.

5. (canceled)

6. The compound of claim 1, wherein L⁴ is —C(CH₃)HO—CH₂—, —CH₂—O—C(CH₃)H—, —CH₂OCH₂—, —CH₂—NH—CH₂—, —CH₂—N(CH₃)—CH₂—, —C(O)NH—S(O)₂—, —CH₂—S(O)₂—CH₂—, —NHC(O)—, —CH₂—O—, —OCH₂—, or

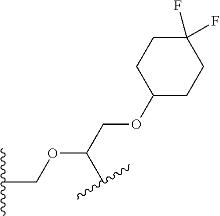 [SMILES: *COC(*)COC1CCC(F)(F)CC1]

7. (canceled)

8. The compound of claim 1, wherein L⁵ is a substituent of Table 2.

9. (canceled)

10. The compound of claim 1, wherein R¹⁰ is a substituent of Table 3.

11-12. (canceled)

13. The compound of claim 1, wherein L² is

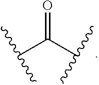 [SMILES: *C(*)=O]

14. (canceled)

15. The compound of claim 1, wherein -L²-R⁶ is a substituent of Table 4 or Table 5.

16. The compound of claim 1, wherein -L²-R⁶ or R⁶ is a substituent of Table 5.

17. (canceled)

18. (canceled)

19. The compound of claim 1, wherein L³ is a saturated or unsaturated, straight or branched, optionally substituted bivalent C₁₋₄ hydrocarbon chain, wherein 0-2 methylene units of L³ are independently replaced by —S(O)₂—, —C(O)NR—, or —C(O)—.

20. (canceled)

21. The compound of claim 1, wherein L³ is a covalent bond.

22. (canceled)

23. The compound of claim 1, wherein R⁸ is selected from Table 7.

24. (canceled)

25. The compound of claim 1 of Formula IIA: 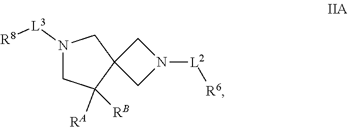
or a pharmaceutically acceptable salt thereof.

26. The compound of claim 1 of Formula IIB: 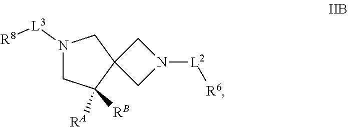
or a pharmaceutically acceptable salt thereof.

27. The compound of claim 1 of Formula IIB′: 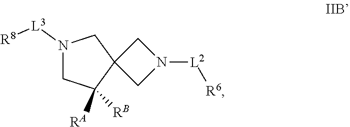
or a pharmaceutically acceptable salt thereof.

28-43. (canceled)

44. A method of treating a disease or disorder associated with CDK2 activity in a patient comprising administering to the patient in need thereof a therapeutic amount of the compound of claim 1, wherein the disease or disorder associated with CDK2 activity is a cancer selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

45-52. (canceled)
